(19) United States
(12) Patent Application Publication
(10) Pub. No.: US 2022/0389513 A1
(43) Pub. Date: Dec. 8, 2022

(54) A Method of Estimating a Circulating Tumor DNA Burden and Related Kits and Methods

(71) Applicant: Agency for Science, Technology and Research, Singapore (SG)

(72) Inventors: Anders Skanderup, SIngapore (SG); Guanhua Zhu, Singapore (SG); Boon Hsi Sarah Ng, Singapore (SG); Bee Huat Iain Tan, Singapore (SG)

(21) Appl. No.: 17/757,159

(22) PCT Filed: Dec. 18, 2020
(86) PCT No.: PCT/SG2020/050766

§ 371 Date: Jun. 10, 2022

(30) Foreign Application Priority Data
Dec. 19, 2019 (SG) 10201912600T

(51) Int. Cl. C12Q 1/6886; G01N 33/574

(52) U.S. Cl. CPC C12Q 1/6886; G01N 33/57419; C12Q 2600/118

(57) ABSTRACT

There is provided a method of estimating a circulating tumour DNA (ctDNA) burden/level in a subject, the method comprising: determining in a blood sample obtained from the subject, a level of cell-free DNA (cfDNA) that maps to one or more nucleosome-depleted region (NDR); and estimating the ctDNA burden based on said level of cfDNA, wherein said NDR (i) comprises the NDR of a gene which transcript is differentially expressed between healthy blood tissue and tumour tissue and/or (ii) is degraded to different extents between healthy blood tissue and blood tissue of a tumour-bearing subject. Also provided are related kits and methods, in one embodiment, the one or more NDR comprises one or more NDR of SLC11A1, NLRP12, PRTN3, HMBS, LILRB3, ACSL1, GP9, MX2, RASGRP4, ATG18L2, SHKBP1, BCAR1, RAB25 and LSR.

### TECHNICAL FIELD

The present disclosure relates broadly to a method of estimating a disease burden, such as a circulating tumor DNA (ctDNA) burden, and related kits and methods.

### BACKGROUND

Cell-free DNA (cfDNA) is present in the blood circulation of humans. In healthy individuals, the death of normal cells of the hematopoietic lineage is the main contributor of plasma cfDNA. In cancer patients, blood plasma can carry circulating tumor DNA (ctDNA) fragments originating from tumor cells, offering non-invasive access to somatic genetic alterations in tumors. The ctDNA profile of a cancer patient is clinically informative in at least two major ways. Firstly, the profile can provide information about specific actionable mutations that can guide therapy. Secondly, the profile can be used to infer tumor growth dynamics by estimating the amount of ctDNA in the blood. This latter information offers a promising non-invasive approach to track disease progression during clinical trials or therapy, offering a real-time tool to adjust therapy.

Existing next-generation sequencing-based approaches to estimate ctDNA levels in plasma samples are based on somatic single nucleotide variant allele frequencies (SNV VAFs), copy number aberrations (CNAs), or DNA methylation patterns. However, these approaches each have limitations.

Approaches based on somatic variant allele frequencies only work for patients that have known recurring cancer mutations, and reliable estimation requires that multiple mutations are present in the ctDNA. Since cfDNA targeted sequencing typically only covers a few hundred selected cancer genes because of the need for ultra-deep sequencing (˜10,000×), most patients will not have a sufficient number of detectable mutations to allow reliable tumor content estimation. ctDNA burden estimation based on SNVs may therefore be challenging when no clonal mutations exist among the targeted genes.

Alternatively, low-pass whole genome sequencing (Ip-WGS) yields segmental/arm-level CNAs, or epigenomics-associated fragmentation patterns that allow for inference of ctDNA burden. However, some cancers may not have sufficient levels of aneuploidy and chromosomal instability needed for robust estimation. Therefore, some cancers cannot be accurately monitored with this approach. Furthermore, low-pass whole genome sequencing approaches only work down to ˜3% tumor ctDNA fraction and the assay must be performed in addition to the standard targeted panel sequencing, wasting precious blood plasma.

Sequencing of DNA methylation patterns may provide a general approach to quantify the cellular origin of cfDNA. However, this technology is less efficient and more noisy (due to bisulfite conversion step) and is again not directly compatible with standard targeted panel sequencing, thereby wasting precious blood plasma.

Notably, both DNA methylation and Ip-WGS profiling require separate assays in addition to standard targeted gene sequencing, highlighting the need for approaches that simultaneously allow for profiling of actionable cancer mutations and quantitative estimation of ctDNA burden.

Thus, there is a need to provide an alternative method of estimating a disease burden, such as a ctDNA burden, and related kits and methods.

### SUMMARY

In one aspect, there is provided a method of estimating a circulating tumor DNA (ctDNA) burden in a subject, the method comprising: determining in a blood sample obtained from the subject, a level of cell-free DNA (cfDNA) that maps to one or more nucleosome-depleted region (NDR); and estimating the ctDNA burden based on said level of cfDNA, wherein said NDR (i) comprises the NDR of a gene which transcript is differentially expressed between healthy blood tissue and tumor tissue and/or (ii) is degraded to different extents between healthy blood tissue and blood tissue of a tumor-bearing subject.

In one embodiment, determining in a blood sample obtained from the subject, a level of cfDNA that maps to one or more NDR comprises: sequencing cfDNA fragments in the blood sample to obtain sequencing reads; and determining the number of sequencing reads that align with the one or more NDR to obtain said level of cfDNA that maps to one or more NDR.

In one embodiment, the method further comprises contacting the blood sample with one or more probe capable of binding to the one or more NDR to capture cfDNA fragments comprising the one or more NDR prior to the sequencing step.

In one embodiment, the NDR is selected from the group consisting of: a promoter region, a first exon-intron junction and combinations thereof.

In one embodiment, the estimated ctDNA burden positively correlates with a tumor burden in the subject.

In one embodiment, said transcript that is differentially expressed between healthy blood tissue and tumor tissue comprises a transcript which FPKM (Fragments Per Kilobase of transcript per Million) value differs by at least 10 times between healthy blood tissue and tumor tissue.

In one embodiment, said NDR that is degraded to different extents between healthy blood tissue and blood tissue of a tumor-bearing subject comprises a NDR having different sequencing coverage in healthy blood tissue and in tumor tissue.

In one embodiment, said transcript that is differentially expressed in healthy blood tissue and tumor tissue is selected from the group consisting of: a transcript that is more highly expressed in healthy blood tissue than in tumor tissue, a transcript that is more highly expressed in tumor tissue than in healthy blood tissue and combinations thereof.

In one embodiment, said transcript which is differentially expressed between blood tissue and tumor tissue consists of transcript(s) that is more highly expressed in blood tissue than in tumor tissue.

In one embodiment, the one or more NDR comprises at least two NDRs, optionally six NDRs, further optionally ten NDRs.

In one embodiment, the total length of the one or more NDR is no more than 30 kb.

In one embodiment, the one or more NDR comprises one or more NDR of a gene selected from the group consisting of: SLC11A1, NLRP12, PRTN3, HMBS, LILRB3, ACSL1, GP9, MX2, RASGRP4, ATG16L2 and combinations thereof.

In one embodiment, the method is a method of determining disease progression in a subject and the method further comprises: determining in a subsequent blood sample obtained from the subject, a level of cfDNA that maps to one or more NDR; estimating the ctDNA burden based on said level of cfDNA; comparing the ctDNA burden estimated from said subsequent blood sample with the ctDNA burden estimated from said blood sample; and identifying the subject as having disease progression if the ctDNA burden estimated from said subsequent blood sample is higher than the ctDNA burden estimated from said blood sample and identifying otherwise if the ctDNA burden estimated from said subsequent blood sample is not higher than the ctDNA burden estimated from said blood sample.

In one embodiment, the method further comprises changing the treatment regimen received by the subject if the subject is identified as having disease progression.

In one embodiment, the tumor comprises colorectal tumor.

In one embodiment, the one or more NDR comprises one or more NDR of a gene selected from the group consisting of: SHKBP1, ACSL1, BCAR1, RAB25, PRTN3, LSR and combinations thereof.

In one aspect, there is provided a kit for estimating a ctDNA burden in a subject, the kit comprising one or more probe that is capable of binding to one or more NDR, wherein said NDR (i) comprises the NDR of a gene which transcript is differentially expressed between healthy blood tissue and tumor tissue and/or (ii) is degraded to different extents between healthy blood tissue and blood tissue of a tumor-bearing subject.

In one embodiment of the kit, said one or more NDR comprises one or more NDR of a gene selected from the group consisting of: SLC11A1, NLRP12, PRTN3, HMBS, LILRB3, ACSL1, GP9, MX2, RASGRP4, ATG16L2 and combinations thereof.

In one embodiment of the kit, said tumor comprises colorectal tumor and said one or more NDR comprises one or more NDR of a gene selected from the group consisting of: SHKBP1, ACSL1, BCAR1, RAB25, PRTN3, LSR and combinations thereof.

In one embodiment of the kit, the one or more probe comprises the sequence of one or more of SEQ ID NO: 1 to SEQ ID NO: 577, or a sequence sharing at least 75% sequence identity thereto.

### Definitions

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases (such as cancer), symptoms and disorders. A medical condition also includes a body's response to a disease or disorder, e.g. inflammation. Those in need of such treatment include those already with a medical condition as well as those prone to getting the medical condition or those in whom a medical condition is to be prevented.

The term “subject” as used herein includes patients and non-patients. The term “patient” refers to individuals suffering or are likely to suffer from a medical condition such as cancer, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a method of estimating a disease burden, such as a ctDNA burden, in a subject and related kits and methods are disclosed hereinafter.

In various embodiments, there is provided a method of estimating, predicting and/or determining one or more of: a disease burden, a cancer burden, a tumor burden, a circulating tumor DNA (ctDNA) burden, a level of ctDNA, an amount of ctDNA, a proportion of ctDNA, a fraction of ctDNA and a ctDNA content in a subject. In various embodiments, the method comprises determining in a sample obtained from the subject, a level, an amount, a proportion, a fraction and/or a content of DNA, optionally cell-free DNA (cfDNA), that aligns with, belongs to, maps to, corresponds to, is similar to and/or identical to at least one genomic region, and estimating, predicting and/or determining one or more of: the disease burden, the cancer burden, the tumor burden, the ctDNA burden, the level of ctDNA, the amount of ctDNA, the proportion of ctDNA, the fraction of ctDNA and/or the ctDNA content in the subject based on the level, the amount, the proportion, the fraction and/or the content of DNA. In some embodiments, the disease burden, the cancer burden, the tumor burden, the ctDNA burden, the level of ctDNA, the amount of ctDNA, the proportion of ctDNA, the fraction of ctDNA and/or the ctDNA content comprises the absolute disease burden, cancer burden, tumor burden, ctDNA burden, level of ctDNA, amount of ctDNA, proportion of ctDNA, fraction of ctDNA and/or ctDNA content.

The estimation, prediction and/or determination may be quantitative, semi-quantitative or qualitative. In various embodiments, the disease burden, the cancer burden, the tumor burden, the ctDNA burden, the level of ctDNA, the amount of ctDNA, the proportion of ctDNA, the fraction of ctDNA and/or the ctDNA content is associated with or correlates with the level, the amount, the proportion, the fraction and/or the content of DNA, optionally cfDNA, in the subject.

In various embodiments, the at least one genomic region comprises a gene. In various embodiments, the at least one genomic region comprises a coding region. In various embodiments, the at least one genomic region comprises a non-coding region (e.g. a region that is far away from genes, a regulatory region such as enhancer etc.). In various embodiments, the at least one genomic region comprises a nucleosome-depleted region (NDR). In various embodiments, the nucleosome-depleted region comprises a gene. In various embodiments, the nucleosome-depleted region comprises a coding region. In various embodiments, the nucleosome-depleted region comprises a non-coding region.

In various embodiments, the at least one genomic region comprises at least one coding region/gene and at least one non-coding region. In various embodiments, determining in the sample a level (or an amount, a proportion, a fraction and/or a content) of DNA that maps to (or aligns with, corresponds to, belongs to, is similar to and/or is identical to) at least one genomic region comprises determining a level of DNA that maps to each of a plurality of genomic regions, the plurality of genomic regions comprising a greater number/proportion of coding region(s)/gene(s) than non-coding region(s). In other words, in various embodiments, the non-coding regions make up a small/minority set of the plurality of regions that are being mapped to.

A NDR may be a region that has a relatively low nucleosome occupancy level. For example, a promoter region upstream of a transcriptional start site (TSS) often displays low nucleosome occupancy level for a typical gene. For example, regulatory regions tend to be nucleosome depleted. In various embodiments, the at least one NDR comprises a transcription start site, a promoter, an intron-exon junction and/or an exon-intron junction. An intron-exon junction may be a first intron-exon junction, a second intron-exon junction, a third intron-exon junction, a fourth intron-exon junction etc. An exon-intron junction may be a first exon-intron junction, a second exon-intron junction, a third exon-intron junction, a fourth exon-intron junction etc. In various embodiments, the NDR is selected from the group consisting of: a promoter region, a first exon-intron junction and combinations thereof. In various examples, cfDNA coverage/degradation pattern at a first exon-intron junction and/or a promoter region is found to possess the capability or better capability to infer gene expression and/or predict ctDNA burden.

In various embodiments, the NDR comprises the NDR of a gene which is differentially expressed in healthy blood tissue/cell and diseased tissue/cell. In various embodiments, the NDR comprises the NDR of a gene which transcript is differentially expressed in healthy blood tissue/cell and diseased tissue/cell. Because a gene usually comprises multiple alternative transcripts with different genomic positions, determining the gene expression at the transcript level (as compared to at the gene level) may allow for a more precise mapping of the NDR e.g. the promoter and junction locations. A gene which transcript is differentially expressed in healthy blood tissue/cell and diseased tissue/cell may be identified by RNA sequencing or any other suitable methods known in the art. A gene which transcript is differentially expressed in healthy blood tissue/cell and diseased tissue/cell may also be identified by analysing transcript expression data available at public databases e.g. the Genotype-Tissue Expression (GTEx) project, The Cancer Genome Atlas (TCGA) program etc. A transcript which is differentially expressed in healthy blood tissue/cell and diseased tissue/cell may have different FPKM (fragments per kilobase of transcript per million mapped fragments/reads) or RPKM (Reads Per Kilobase of transcript, per Million mapped reads), or TPM (Transcripts Per Million) values in healthy blood tissue/cell and in diseased tissue/cell (e.g. as determined by sequencing).

In various embodiments, the difference in the expression or FPKM/RPKM/TPM value of the transcript in healthy blood tissue/cell and in diseased tissue/cell is at least about 10%, at least about 20%, at least about 30%, at least about 40%, at least about 50%, at least about 60%, at least about 70%, at least about 80%, at least about 90% or at least about 100%. In various embodiments, the difference in the expression of the transcript FPKM/RPKM/TPM value in healthy blood tissue/cell and in diseased tissue/cell is at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold, at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold or at least about 15 fold. In various embodiments, the difference in the expression of the transcript FPKM/RPKM/TPM value in healthy blood tissue/cell and in diseased tissue/cell is at least about 0.1 times, at least about 0.2 times, at least about 0.3 times, at least about 0.4 times, at least about 0.5 times, at least about 0.6 times, at least about 0.7 times, at least about 0.8 times, at least about 0.9 times, at least about 1 times, at least about 2 times, at least about 3 times, at least about 4 times, at least about 5 times, at least about 6 times, at least about 7 times, at least about 8 times, at least about 9 times, at least about 10 times, at least about 11 times, at least about 12 times, at least about 13 times, at least about 14 times or at least about 15 times. In various embodiments, the FPKM/RPKM/TPM value comprises a median FPKM/RPKM/TPM value obtained from a plurality of healthy blood tissue/cell samples and/or a plurality of diseased tissue/cell samples.

In various embodiments, the NDR is degraded to different extents in healthy blood tissue/cell and in blood tissue/cell of a diseased subject. In various embodiments, the NDR has different degradation patterns/signals in healthy blood tissue/cell and in blood tissue/cell of a diseased subject. For example, when sequencing cfDNA in a healthy blood tissue/cell sample and in blood tissue/cell sample of a diseased subject, a greater or smaller number/amount (i.e. a substantially different or non-identical number/amount) of fragments/reads may map to the NDR in the healthy blood tissue/cell sample as compared to the blood tissue/cell sample of the diseased subject. For example, when sequencing cfDNA in a healthy blood tissue/cell sample and in a blood tissue/cell sample of a diseased subject, the read depth or coverage of the NDR may be higher or lower in the healthy blood tissue/cell sample as compared to the blood tissue/cell sample of a diseased subject. In various embodiments therefore, the NDR has different (or non-similar or non-identical) read depth or coverage in healthy blood tissue/cell and in blood tissue/cell of a diseased subject.

The read depth or coverage of a NDR may comprise a relative read depth or relative coverage of the NDR. A relative read depth or relative coverage of a NDR may be obtained, for example, by normalizing/dividing the raw read depth/coverage across the NDR (or optionally a mean raw read depth/coverage across the NDR for multiple samples/runs) by a normalization factor. In one example, the normalization factor comprises the read depth or coverage (or optionally a mean read depth/coverage for multiple samples/runs) of region(s) flanking the NDR e.g. the flanking upstream and/or downstream regions. In one example, the normalization factor is the mean coverage of the upstream and downstream flanks of the NDR. In one example therefore, the relative read depth or relative coverage of a NDR is the mean raw read depth/coverage across the NDR divided by the mean raw read depth/coverage of the upstream and downstream flanks.

In some embodiments, the flanking region(s) is immediately upstream or downstream of the NDR, or contiguous with the NDR. In some embodiments, the flanking region(s) is separated from the NDR by one or more nucleotides/bases. In various embodiments, the flanking region(s) is no more than about 5000 base pairs (bp), no more than about 4500 bp, no more than about 4000 bp, no more than about 3500 bp, no more than about 3000 bp, no more than about 2500 bp or no more than about 2000 bp from the NDR or an end of the NDR. In various embodiments, the flanking region(s) is at least about 50 bp, at least about 100 bp, at least about 150 bp, at least about 200 bp, at least about 250 bp, at least about 300 bp, at least about 350 bp, at least about 400 bp, at least about 450 bp, at least about 500 bp, at least about 550 bp, at least about 600 bp, at least about 650 bp, at least about 700 bp, at least about 750 bp, at least about 800 bp, at least about 850 bp, at least about 900 bp, at least about 950 bp, or least about 1000 bp from the NDR or an end of the NDR.

In various embodiments, the size/length of flanking region(s) is at least about 50 bp, at least about 100 bp, at least about 150 bp, at least about 200 bp, at least about 250 bp, at least about 300 bp, at least about 350 bp, at least about 400 bp, at least about 450 bp, at least about 500 bp, at least about 550 bp, at least about 600 bp, at least about 650 bp, at least about 700 bp, at least about 750 bp, at least about 800 bp, at least about 850 bp, at least about 900 bp, at least about 950 bp, or least about 1000 bp.

In one example, the NDR is about −300 bp to about 300 bp, about −200 bp to about 100 bp or about −150 bp to about 50 bp relative to a transcription start site (TSS) and the normalization factor is the mean coverage of an upstream flank that is about −2000 bp to about −1000 bp relative to the TSS and a downstream flank that is about 1000 bp to about 2000 bp relative to the TSS.

In various embodiments, a NDR that is degraded to different extents in healthy blood tissue/cell and in blood tissue/cell of a diseased subject may be identified by comparing the relative depth/coverage of the NDR in healthy blood tissue/cell and in the blood tissue/cell of a diseased subject. For example, if the relative depth/coverage of the NDR in healthy blood tissue/cell and in the blood tissue/cell of a diseased subject are different, the NDR is considered to a NDR that is degraded to different extents in healthy blood tissue/cell and in blood tissue/cell of a diseased subject. In various embodiments, determining the relative depth/coverage of a NDR in healthy blood tissue/cell and/or in blood tissue/cell of a diseased subject comprises determining the coverage of each position in an about 8 k-bp window, about 6 k-bp window, about 4 k-bp window, about 2 k-bp window or about 1 k-bp window spanning from about −4000 to +4000 bp, from about −3000 to +3000 bp, from about −2000 to +2000 bp, from about −1000 to +1000 bp or from about −500 to +500 bp with respect the NDR (e.g. end(s) of the NDR); and optionally normalizing the coverage by the mean coverage of the upstream region (e.g. −8000 to −4000 bp, −4000 to −2000 bp, −3000 to −1000 bp, −2000 to −1000 bp or −1000 to −500 bp with respect to the NDR (e.g. end(s) of the NDR)) and/or downstream region (e.g. +4000 bp to +8000 bp, +2000 to +4000 bp, +1000 to +3000 bp+1000 to +2000 bp or +500 to +1000 bp with respect to the NDR (e.g. end(s) of the NDR) to obtain a relative depth/coverage for the NDR. In some examples, the coverage of each position in a region located downstream of a NDR (e.g. a promoter) is determined. In some examples, the coverage of each position in a region located from about −350 bp to about −50 bp or from about −300 to about −100 bp with respect a NDR (e.g. an end of a first exon) is determined.

In various embodiments, the difference in read depth or coverage (or relative read depth or coverage) in healthy blood tissue/cell and in blood tissue/cell of a diseased subject is measured by computing a coverage score (or relative coverage score). In various embodiments, the coverage score (or relative coverage score) is computed by the following formula:

$$score = \frac{mean ( diseased ) - mean ( healthy )}{\sqrt{s . d . ( diseased )}}$$

where mean(diseased) and mean(healthy) are the mean of average coverages (or relative coverages) at NDRs across diseased blood tissue/cell (e.g. plasma samples of diseased subjects) and healthy blood tissue/cell (e.g. healthy plasma samples) respectively, and s.d. (diseased) is the standard deviation of average coverages (or relative coverages) at NDRs across diseased blood tissue/cell.

In various embodiments, the coverage values negatively correlate with expression level. In some examples therefore, blood genes/transcripts (e.g. genes/transcripts show a higher FPKM value in normal blood than in tumor) have a higher coverage in diseased samples than in healthy samples. Thus, the blood genes/transcripts have a positive value of relative coverage score, as mean(diseased)>mean(healthy). In some examples, tumor genes/transcripts (e.g. genes/transcripts show a higher FPKM value in tumor than in normal blood) have a lower coverage in disease samples than in healthy samples. Thus, the tumor genes have a negative value of relative coverage score, as mean(diseased)<mean(healthy).

In various embodiments, the NDR has a coverage score or relative coverage score of less than about 0 and/or more than about 0. In various embodiments, the NDR has a coverage score or relative coverage score of less than about −0.1, less than about −0.2, less than about −0.3, less than about −0.4, less than about −0.5, less than about −0.6, less than about −0.7, less than about −0.8, less than about −0.9 or less than about −1.0. In various embodiments, the NDR has a coverage score or relative coverage score of more than about 0.1, more than about 0.2, more than about 0.3, more than about 0.4, more than about 0.5, more than about 0.6, more than about 0.7, more than about 0.8, more than about 0.9 or more than about 1.0.

As used herein, “blood”, “blood tissue” or “blood sample” refers to whole blood or fractions thereof, such as a plasma fraction or a serum fraction. As used herein “healthy blood”, “healthy blood tissue” or “healthy blood sample” refers to the whole blood or fractions thereof of a healthy subject, or a subject who does not suffer from the disease. Conversely, “diseased blood”, “diseased blood tissue” or “diseased blood sample” as used herein refers to the whole blood or fractions thereof of a diseased subject, or a subject who suffers from the disease. In various embodiments, “diseased blood”, “diseased blood tissue” or “diseased blood sample” does not indicate that a disease necessarily resides in the blood per se. For example, “diseased blood”, “diseased blood tissue” or “diseased blood sample” may refer to the blood, tissue or sample of a subject suffering from colorectal cancer and having no blood diseases, and “healthy blood”, “healthy blood tissue” or “healthy blood sample” may refer to the blood, tissue or sample of a subject who does not suffer from colorectal cancer.

In various embodiments, the sample obtained from the subject comprises a liquid sample. In various embodiments, the sample comprises a biological fluid sample. In various embodiments, the liquid/biological fluid sample comprises one or more of blood, serum, plasma, sputum, lavage fluid, cerebrospinal fluid, interstitial fluid, urine, feces, milk, semen, sweat, tears, saliva, and the like. In various embodiments, the sample comprises a blood sample (e.g. whole blood sample or processed fractions thereof). In various embodiments, the sample comprises a plasma sample. In various embodiments, the sample comprises cfDNA. In various embodiments, the sample comprises cfDNA, for example, cfDNA extracted/isolated/purified from a blood sample obtained from the subject.

In various embodiments, the disease comprises a proliferative disease and the diseased tissue/cell comprises a proliferative tissue/cell. In various embodiments, the disease comprises a malignant disease and the diseased tissue/cell comprises a malignant tissue/cell. In various embodiments, the malignant disease comprises cancer and the diseased tissue/cell comprises a cancer tissue/cell. In various embodiments, the cancer comprises solid tumor cancers.

In various embodiments therefore, there is provided a method of estimating a ctDNA burden in a subject, the method comprising: determining in a blood sample obtained from the subject, a level of cfDNA that maps to one or more nucleosome-depleted region (NDR); and estimating the ctDNA burden based on said level of cfDNA, wherein said NDR (i) comprises the NDR of a gene which transcript is differentially expressed between healthy blood tissue and tumor tissue and/or (ii) is degraded to different extents between healthy blood tissue and blood tissue of a tumor-bearing subject. Advantageously, a level of cfDNA that maps to selected NDR(s) is identified to be a good estimator of or proxy for tumor burden or ctDNA burden.

In various embodiments, the estimated ctDNA burden associates/correlates, optionally positively associates/correlates with a tumor burden in the subject. In various embodiments, the higher the estimated ctDNA burden in the subject, the higher the tumor burden in the subject. In various embodiments, the higher the estimated ctDNA burden in the subject, the higher the estimated amount of cancer/tumor cells in the subject. In various embodiments, the higher the estimated ctDNA burden in the subject, the higher the estimated mass/size/volume of tumor in the subject. In various embodiments, the association/correlation, optionally positive association/correlation, may be linear (i.e. the ratio of change is constant) or non-linear (i.e. the ratio of change is not constant).

In various embodiments, the estimated ctDNA burden is associated/correlated with the level of cfDNA that maps to one or more NDRs. The association/correlation may be positive and/or negative, linear and/or non-linear and monotonic and/or non-monotonic. For example, the estimated ctDNA burden may be positively associated/correlated with the level of cfDNA that maps to a first NDR and negatively associated/correlated with the level of cfDNA burden that maps to a second NDR. For example, the estimated ctDNA burden may be linearly associated/correlated (e.g. positive or negative) with the level of cfDNA that maps to a first NDR and non-linearly associated/correlated with the level of cfDNA burden that maps to a second NDR. For example, the estimated ctDNA burden may be monotonically associated/correlated with the level of cfDNA that maps to a first NDR and non-monotonically associated/correlated with the level of cfDNA that maps to a second NDR.

In one example, the signs of the coefficients for the one or more NDRs in a trained model correspond to the sign of the differential expression of the associated transcripts in tumor tissue relative to healthy blood tissue. In various embodiments, an NDR associated with a cancer-specific gene/transcript or a tumor gene/transcript (e.g. a gene/transcript that shows a higher FPKM value in tumor than in normal blood) has a negative coefficient/correlation with the estimated ctDNA burden. In various embodiments, an NDR associated with a blood gene/transcript (e.g. a gene/transcript that shows a higher FPKM value in normal blood than in tumor) has a positive coefficient/correlation with the estimated ctDNA burden. In various embodiments, the estimated ctDNA burden is negatively associated/correlated with a level of cfDNA that maps to one or more NDR of a gene which transcript is more highly expressed in tumor tissue than in healthy blood tissue and/or the estimated ctDNA burden is positively associated/correlated with a level of cfDNA that maps to one or more NDR of a gene which transcript is more highly expressed in healthy blood tissue than in tumor tissue. In some embodiments, the estimated ctDNA burden is linearly correlated with the level of cfDNA that maps to one or more NDRs.

In various embodiments, the determining step comprises sequencing the DNA or cfDNA present in the blood sample obtained from the subject. Examples of sequencing techniques include next-generation sequencing, amplicon-based sequencing, paired-end sequencing, Sanger sequencing etc. In some embodiments, sequencing the DNA or cfDNA present in the blood sample comprises subjecting the DNA or cfDNA present in the blood sample to deep sequencing. In one embodiment, sequencing the DNA or cfDNA present in the blood sample comprises subjecting the DNA or cfDNA present in the blood sample to next-generation sequencing. In some examples, deep sequencing is performed such that the depth/coverage at the one or more NDR/at least one NDR is at least about 10×, at least about 25×, at least about 50×, at least about 100×, at least about 200×, at least about 300×, at least about 400×, at least about 500×, at least about 600×, at least about 700×, at least about 800×, at least about 900× or at least about 1000×, at least about 2000×, at least about 3000×, at least about 4000×, at least about 5000× or at least about 6000×. In various embodiments, the sequencing does not comprise ultra-deep sequencing. In various embodiments, the depth/coverage at the one or more NDR/at least one NDR is or is kept to less than about 10,000×, less than about 9000×, less than about 8000×, less than about 7000×, less than about 6000×, less than about 5000×, less than about 4000×, less than about 3000×, less than about 2000× or less than about 1000×. In various embodiments, the depth/coverage at the one or more NDR/at least one NDR is or is kept to no more than about 10,000×, no more than about 9000×, no more than about 8000×, no more than about 7000×, no more than about 6000×, no more than about 5000×, no more than about 4000×, no more than about 3000×, no more than about 2000× or no more than about 1000×.

In various embodiments, determining in a blood sample obtained from the subject, a level of cfDNA that maps to one or more NDR comprises sequencing cfDNA/cfDNA fragments in the blood sample to obtain sequencing reads; and determining the number of sequencing reads that align with the one or more NDR to obtain said level of cfDNA that maps to one or more NDR. In various embodiments, determining in a blood sample obtained from the subject, a level of cfDNA that maps to one or more NDR comprises sequencing any cfDNA/cfDNA fragments present in the blood sample and determining the depth/read depth/coverage/sequencing coverage at the one or more NDR. The depth/read depth/coverage may be a relative depth/read depth/coverage/sequencing coverage. For example, the depth/read depth/coverage/sequencing coverage may be normalized/divided by a normalization factor, for example, a normalization factor as described herein, to obtain the relative depth/read depth/coverage/sequencing coverage. In one example, the relative depth/read depth/coverage/sequencing coverage is obtained by dividing/normalizing the depth/read depth/coverage/sequencing coverage (or mean depth/read depth/coverage/sequencing coverage) across the one or more NDR by the depth/read depth/coverage/sequencing coverage (or mean depth/read depth/coverage/sequencing coverage) of an upstream flank and/or a downstream flank, for example, an upstream flanking region and/or a downstream flanking region as described herein. In some embodiments therefore, the method further comprises determining the number of sequencing reads that align with one or more regions flanking the one of more NDR. In some embodiments, the method further comprises determining the depth/read depth/coverage/sequencing coverage at the one or more region flanking the one of more NDR.

The sequencing may be targeted or untargeted. Where the sequencing comprises targeted sequencing, probe(s) may be used to capture and isolate specific genomic regions for sequencing. In some embodiments therefore, the method further comprises contacting the blood sample with one or more probe capable of binding to the one or more NDR to capture cfDNA/cfDNA fragments comprising the one or more NDR prior to the sequencing step.

In various embodiments, determining in a blood sample obtained from the subject, a level of cfDNA that maps to one or more NDR comprises performing quantitative polymerase chain reaction (qPCR) or real-time polymerase chain reaction (real-time PCR) to determine the amount/proportion of cfDNA that maps to one or more NDR. In various embodiments, the performing step comprises contacting the sample with a primer that is capable of hybridizing/binding (e.g. under stringent conditions) to or a primer that is specific to the one or more NDR.

In various embodiments, the method further comprises amplifying the cfDNA in the blood sample. The amplification step may be carried out before the step of determining a level of cfDNA. The amplification step may also be carried out before the step of sequencing cfDNA/cfDNA fragments in the blood sample and/or before the step of contacting the blood sample with the one or more probe. Amplification reactions known in the art may be employed. The amplification reactions may include but are not limited to polymerase chain reaction (PCR), ligase chain reaction (LCR), loop mediated isothermal amplification (LAMP), nucleic acid sequence based amplification (NASBA), self-sustained sequence replication (3SR), rolling circle amplification (RCA) or any other process whereby one or more copies of a particular polynucleotide sequence or nucleic acid sequence may be generated from a polynucleotide template sequence or nucleic acid template sequence.

In various embodiments, the method further comprises processing the cfDNA and/or its associated data. In various embodiments, the cfDNA are trimmed at one or both ends to retain only a central region and/or data associated with a central region of the cfDNA. Advantageously, trimming the cfDNA and/or its associated data from one or both ends to retain only a central region and/or data associated with a central region of the cfDNA may amplify a degradation signal and/or increases a coverage signal. In various embodiments, the trimmed cfDNA/central region is no more than about 70 bp, no more than about 60 bp or no more than about 50 bp in length. In various embodiments, the trimmed cfDNA/central region is about 70 bp, about 60 bp or about 50 bp in length. In one embodiment, the central region is about 61 bp. The method may also work with an untrimmed cfDNA (e.g. a cfDNA of about 151 bp), although the signal produced may be weaker.

In various embodiments, the cfDNA and/or its associated data are trimmed in-silico e.g. by use of the software BamUtil. In various embodiments, the cfDNA and/or its associated data are trimmed after sequencing.

In various embodiments, said NDR that is degraded to different extents between healthy blood tissue and blood tissue of a tumor-bearing subject comprises a NDR having different depth/read depth/coverage/sequencing coverage in healthy blood tissue and in tumor tissue.

In various embodiments, said transcript that is differentially expressed between healthy blood tissue and tumor tissue comprises a transcript which FPKM value differs by at least about 2 times, at least about 3 times, at least about 4 times, at least about 5 times, at least about 6 times, at least about 7 times, at least about 8 times, at least about 9 times or at least about 10 times between healthy blood tissue and tumor tissue (e.g. as determined by sequencing).

In various embodiments, said transcript that is differentially expressed between healthy blood tissue and tumor tissue comprises a transcript which FPKM value in healthy blood tissue is less than about 30, less than about 20, less than about 10, less than about 5, less than about 3, less than about 1, less than about 0.5, less than about 0.1, less than about 0.05 or less than about 0.01. In one embodiment, the FPKM value of the transcript in healthy blood tissue in less than about 1. In various embodiments, the FPKM value of the transcript in healthy blood tissue is more than about 0.01, more than about 0.05, more than about 0.1, more than about 0.5, more than about 1, more than about 3, more than about 5, more than about 10, more than about 20 or more than about 30. In one embodiment, the FPKM value of the transcript in healthy blood tissue is more than about 10. In various embodiments, the FPKM value of the transcript in healthy blood tissue is between about 0.01 and about 0.1, between about 0.1 and about 1, between about 1 and about 5 or between about 5 and about 30.

In various embodiments, said transcript that is differentially expressed between healthy blood tissue and tumor tissue comprises a transcript which FPKM value in tumor tissue is less than about 30, less than about 20, less than about 10, less than about 5, less than about 3, less than about 1, less than about 0.5, less than about 0.1, less than about 0.05 or less than about 0.01. In one embodiment, the FPKM value of the transcript in tumor tissue in less than about 1. In various embodiments, the FPKM value of the transcript in tumor tissue is more than about 0.01, more than about 0.05, more than about 0.1, more than about 0.5, more than about 1, more than about 3, more than about 5, more than about 10, more than about 20 or more than about 30. In one embodiment, the FPKM value of the transcript in tumor tissue is more than about 10. In various embodiments, the FPKM value of the transcript in tumor tissue is between about 0.01 and about 0.1, between about 0.1 and about 1, between about 1 and about 5 or between about 5 and about 30.

Some transcripts may be more highly expressed in healthy blood tissue than tumor tissue. In various embodiments, a blood transcript comprises a transcript that is more highly expressed in healthy blood tissue than tumor tissue. Some transcripts may be more highly expressed in tumor tissue than blood tissue. In various embodiments, a tumor transcript comprises a transcript that is more highly expressed in tumor tissue than blood tissue. The one or more NDR may comprise at least about 10%, at least about 20%, or at least about 30%, at least about 40%, at least about 50%, at least about 60%, at least about 70%, at least about 80%, at least about 90% or about 100% NDRs which transcripts more highly expressed in healthy blood tissue than tumor tissue. The one or more NDR may comprise at least about 10%, at least about 20%, or at least about 30%, at least about 40%, at least about 50%, at least about 60%, at least about 70%, at least about 80%, at least about 90% or at about 100% NDRs which transcripts more highly expressed in tumor tissue than in blood tissue. The one or more NDR may comprise at least about one, at least about two or at least about three NDRs which transcripts more highly expressed in healthy blood tissue than tumor tissue and/or at least about one, at least about two or at least about three NDRs which transcripts are more highly expressed in tumor tissue than in blood tissue.

In various embodiments, said transcript which is differentially expressed in healthy blood tissue and tumor tissue is selected from the group consisting of: a transcript that is more highly expressed in healthy blood tissue than tumor tissue, a transcript that is more highly expressed in tumor tissue than healthy blood tissue and combinations thereof. In one embodiment, said transcript which is differentially expressed between blood tissue and tumor tissue consists of transcript(s) that is more highly expressed in tumor tissue than blood tissue. In one embodiment, said transcript which is differentially expressed between blood tissue and tumor tissue consists of transcript(s) that is more highly expressed in blood tissue than tumor tissue. In one embodiment, said transcript does not comprise a transcript which is more highly expressed in tumor tissue than blood tissue. Without being bound by theory, it is believed that tumor-derived DNA component in cancer plasma weakens the blood-specific DNA degradation pattern, and thus the decay of blood-specific signal (alone i.e. without determining the signal of any tumor-associated genes) may be used to robustly estimate a ctDNA content, regardless of cancer types.

In various embodiments therefore, the method is suitable for estimating a disease burden for a specific cancer type, a specific group of cancers, or for all cancers in general (i.e. pan-cancer). In various embodiments, the method comprises a method of estimating a ctDNA burden or tumor burden associated with one or more of the following cancers: bladder cancer, bladder urothelial carcinoma, breast cancer, breast invasive carcinoma, cervical cancer, cervical squamous cell carcinoma, endocervical adenocarcinoma, colorectal cancer, esophageal cancer, esophageal carcinoma, brain cancer, glioblastoma multiforme, head and neck cancer, head and neck squamous cell carcinoma, kidney cancer, renal cell cancer, kidney renal clear cell carcinoma, kidney renal papillary cell carcinoma, brain lower grade glioma, liver cancer, liver hepatocellular carcinoma, lung cancer, lung adenocarcinoma, lung squamous cell carcinoma, ovarian cancer, ovarian serous cystadenocarcinoma, pancreatic cancer, pancreatic adenocarcinoma, prostate cancer, prostate adenocarcinoma, skin cancer, skin cutaneous melanoma, gastric cancer, stomach cancer, stomach adenocarcinoma, thyroid cancer, thyroid carcinoma, endometrial cancer, uterine cancer, uterine corpus endometrial carcinoma, reproductive cancers, gastrointestinal cancers, respiratory cancers, or subtypes thereof. Thus, in various embodiments, the subject has or suffers from one or more of these cancers. In various embodiments, the tumor-bearing subject bears one or more of these tumors. In various embodiments, the subject or tumor-bearing subject does not have or does not suffer from blood cancer/hematologic cancer/hematologic malignancy.

In one embodiment, the method comprises a method of estimating a tumor burden associated with colorectal cancer. In one embodiment, the subject has or suffers from colorectal cancer. In one embodiment, the tumor-bearing subject bears a colorectal tumor. In one embodiment, the method comprises a method of estimating a ctDNA burden or tumor burden associated with breast cancer. In one embodiment, the subject has or suffers from breast cancer. In one embodiment, the tumor-bearing subject bears a breast tumor.

In one embodiment, where the method comprises a method of estimating a ctDNA burden or tumor burden associated with a specific cancer type or a specific group of cancers, the NDR comprises at least one NDR of a gene which transcript shows a higher FPKM value in tumor belonging to the specific cancer type or the specific group of cancers than in healthy/normal blood. In some examples, the transcript has a FPKMₜᵤₘₒᵣ>about 5 or >about 10 and a FPKM_{blood}<about 1. In one embodiment, where the method comprises a method of estimating a ctDNA burden or tumor burden associated with any cancer in general (e.g. pan-cancer), the NDR comprises at least one NDR of a gene which transcript shows a higher FPKM value in normal blood than in tumor. In some examples, the transcript has a FPKM_{blood}>about 5 or >about 10 and a FPKMₜᵤₘₒᵣ<about 1. In various embodiments, where the method comprises a method of estimating a ctDNA burden or tumor burden associated with any cancer in general (e.g. pan-cancer), the NDR consists of NDR(s) of gene(s) which transcript(s) shows a higher FPKM value in normal blood than in tumor.

In various embodiments, the one or more NDR comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19 or at least about 20 NDRs. In various embodiments, the one or more NDR comprises the NDR of at least about two, at least about three, at least about four, at least about five, at least about six, at least about seven, at least about eight, at least about nine, at least about ten genes, at least about 11, at least about 12, at least about 13, at least about 14, at least about 15, at least about 16, at least about 17, at least about 18, at least about 19 or at least about 20 genes or distinct genes.

In some embodiments, the one or more NDR comprises at least about two NDRs, optionally about six NDRs, further optionally about ten NDRs. In some embodiments, the one or more NDR comprises the NDR of at least about two genes (or distinct genes), optionally about six genes (or distinct genes), further optionally about ten genes (or distinct genes). In some embodiments, the one or more NDR comprises at least about four NDRs or the NDRs of at least about four genes or distinct genes. In some embodiments, the one or more NDR comprises no more than about nine NDRs or NDRs of no more than about nine genes or distinct genes. In some embodiments, the one or more NDR comprises about four to about nine NDRs or NDRs of about four to about nine genes or distinct genes. In some embodiments, the one or more NDR comprises about six NDRs or NDRs of about six genes or distinct genes. In some embodiments, the one or more NDR comprises no more than about 13 NDRs or NDRs of no more than about 13 genes or distinct genes. In some embodiments, the one or more NDR comprises about nine, about 10, about 11, about 12 or about 13 NDRs or NDRs of about nine, about 10, about 11, about 12 or about 13 genes or distinct genes. The suitable number of NDRs, genes or features may be further varied, and is within the purview of a person skilled in the art. The number or the reasonable range of numbers of NDRs, genes or features may be determined, for example, by checking an error evolution with the number of top predictive genes or features (e.g. genes or features that are selected most frequently as being predictive by a machine learning model in multiple iterations).

In various embodiments, the NDRs/genes comprises one or more NDRs/genes listed in one or more of Table 1, Table 2, Table S3, Table S10, Table S14, Table S15, Table S16, Table S17, Table S18, Table S19, Table S20 and Table S21.

In various embodiments, the NDRs/genes comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ABHD5, ABTB1, ACAP1, AC01, ACRBP, ACSL1, ADAM8, ADIRF, AGR2, AGR3, AHSP, AK2, AKNA, ALAS2, ALDH18A1, ALOX5, ANKS4B, ANPEP, AOAH, APOBEC3A, ARAP1, ARHGAP25, ARHGAP26, ARHGAP30, ARHGAP9, ARHGEF16, ARHGEF35, ARIDSA, ARRB2, ARSE, ATG16L2, ATP2A2, ATP2C2, ATP5G1, ATP5G3, ATP6V1B2, AXIN2, AZGP1, AZU1, B3GNT3, BATF2, BCAR1, BCL2L15, BCL2L2, BCL6, BDH1, BDH2, BEST1, BGN, BIN2, BIRCS, BMP4, BMX, BOK, BPI, BSPRY, BTK, BTNL8, C10orf54, C11orf21, C16orf54, C19orf33, C19orf35, C1orf162, C1orf210, C1orf228, C1QTNF5, C3, C5AR2, C6orf203, C6orf25, C8orf59, CA1, CA4, CALD1, CAMP, CAPNS, CARS2, CCDC88B, CCL20, CCM2, CCND3, CCR7, CD177, CD244, CD276, CD300E, CD300LB, CD300LF, CD37, CD44, CD53, CD55, CDC42SE1, CDCP1, CDH1, CDH17, CDHRS, CDK4, CDK5RAP2, CDX1, CEACAM1, CEACAM3, CEACAM4, CEACAM5, CELF2, CENPF, CFD, CFP, CFTR, CHCHD6, CHID1, CKB, CKMT1B, CLC, CLDN7, CLEC12A, CLEC4D, CLEC4E, CMTM2, CNN2, CORO1A, CORO7, COTL1, COX6C, CR1, CRB3, CSF3R, CTGF, CTNND1, CXCR1, CXCR2, CYBA, CYTH4, DDC, DDR1, DDX10, DEF8, DEFA1, DEFA1B, DEFA3, DEFA4, DENND1C, DENND3, DHRS13, DHX34, DMTN, DNAH17, DOCK2, DOK3, DPEP2, DYSF, ECE1, ECT2, EEF1E1, EFNA3, EGLN2, E124, ELANE, ELF3, EMP1, ENTPD2, ENTPD6, EPB42, EPCAM, EPHA2, EPS8L3, ERBB2, ERBB3, EVI2B, F3, FAM101A, FAM109A, FAM212B, FAM213B, FAM49B, FAM60A, FAM65B, FAM83E, FAM84A, FBL, FBXL5, FCAR, FCGR2A, FCGR3B, FCN1, FERMT1, FERMT3, FES, FFAR2, FGD3, FGFR4, FGFRL1, FGR, FKBP8, FLOT2, FMNL1, FN1, FOLR3, FOXA2, FPR1, FPR2, FUT2, FUT6, FUT7, GATA1, GBP3, GCA, GGT6, GJB2, GLRX3, GLT1D1, GMFG, GMNN, GNG2, GNLY, GOLT1A, GP9, GPC4, GPR35, GPRCSA, GPSM3, GPX2, GRAMD1A, GRAP2, GRTP1, GZMH, H2AFY2, HBA1, HBA2, HBB, HBD, HBG1, HBG2, HBM, HBQ1, HCK, HCLS1, HID1, HK3, HKDC1, HMBS, HMGB3, HMGCS2, HN1L, HNF4A, HTRA1, ICAM3, IFI30, IFITM1, IFITM2, IFT172, IKZF1, IL16, IL18RAP, IL1R2, IL1 RN, IL2RG, IL32, ILVBL, IMPDH1, INPP5D, IPO5, ITGA2B, ITGAL, ITGAM, ITGAX, ITGB2, ITGB4, JAK3, JUND, JUP, KCNAB2, KCNE1, KIAA1191, KIFC1, KLF1, LAD1, LAMB2, LAMC2, LAPTM5, LCP2, LDHA, LGALS3BP, LGALS4, LGMN, LILRA1, LILRA5, LILRB2, LILRB3, LIMD2, LIPH, LMNB1, LMO7, LPAR2, LRCH4, LRRC25, LSP1, LSR, LST1, LTB, LYL1, MACROD1, MAGED1, MAN2A2, MAP1LC3A, MEFV, MEP1A, MFAP4, MIS18A, MISP, MKNK1, MLKL, MMAB, MME, MMP11, MMP25, MMP7, MMP8, MORN2, MPO, MPP1, MPZL2, MRPL17, MSL3, MSRB1, MTIF2, MUC13, MX2, MXD3, MYL4, MYO1A, MYO1F, MYO1G, MZT2A, NABP1, NADK, NAIP, NAMPT, NARF, NBEAL2, NCF1, NCF2, NCF4, NDEL1, NDUFAF4, NDUFB5, NEK2, NFAM1, NFE2, NKG7, NLRC4, NLRC5, NLRP12, NNMT, NOX1, NQO1, NTMT1, NTPCR, NUPR1, OAZ1, ORM1, OSCAR, P2RX1, PADI2, PADI4, PALLD, PARVG, PDHA1, PDHX, PGD, PGLYRP1, PHC2, PHF21A, PHGR1, PHOSPHO1, PIK3R5, PIN4, PLB1, PLBD1, PLCB2, PLCD3, PLCG2, PLEKHA1, PLS3, POF1B, POLR2H, POSTN, PPBP, PPIL1, PPM1M, PPP1R16A, PPP1R1B, PRAM1, PRAP1, PREX1, PRKCB, PROCR, PROK2, PRR15, PRR15L, PRRC1, PRSS22, PRSS8, PRTN3, PSTPIP1, PTK2B, PTPN6, PYCR1, PYGL, R3HDM4, RAB24, RAB25, RAC2, RARRES2, RASAL3, RASGRP2, RASGRP4, RASSF2, REG4, RELT, REM2, RETN, RFC3, RGL4, RGS19, RHOD, RHPN2, RIN3, RNASE4, RND3, RNF166, RNF167, RPL41, RPS6KA1, RPS9, S100A12, S100A14, S100A8, S1PR4, SAP25, SASH3, SCNN1A, SCOC, SDCBP2, SEC11C, SECTM1, SELL, SEMA4D, SEPP1, SEPTIN10, SFN, SHKBP1, SIGLEC5, SIPA1, SIRPB1, SLC11A1, SLC12A9, SLC16A3, SLC25A37, SLC2A3, SLC2A8, SLC38A5, SLC39A5, SLC43A2, SLCO3A1, SMAP2, SMIM22, SNCA, SORD, SORL1, SPATS2L, SPI1, SRC, ST20, STAP2, STARD10, STAT5B, STEAP1, STK10, STX11, STXBP2, SULT2B1, SYTL3, TACC3, TAGAP, TALDO1, TBC1 D10C, TBX21, TBXAS1, TCEAL4, TFF3, THBS2, THEMIS2, TIMM8B, TINAGL1, TJP3, TLR6, TM4SF5, TMBIM6, TMC4, TMCC2, TMEM106C, TMEM126B, TMEM14A, TMEM14C, TMEM71, TMEM91, TMEM97, TMPRSS2, TMPRSS4, TNFRSF10C, TNS3, TOP2A, TPM1, TRAF3IP3, TREM1, TREML2, TRIM22, TRIM25, TSKU, TSPAN15, TSPAN8, TUBA4A, TUBB1, TYMS, TYROBP, UBE2C, UBE2D3, UBE2T, UGT8, UNC13D, UQCC2, URI1, USB1, USH1C, VARS, VASP, VAV1, VMP1, VNN2, VNN3, VPS51, VSTM1, VWA1, WAS, WDR12, WFS1, XPO6, ZAP70, ZDHHC18, ZDHHC19, ZDHHC9, ZNF467, ZWINT and parts thereof.

In some embodiments, the one or more NDR comprises one or more NDR of a gene selected from the group consisting of: SHKBP1, ACSL1, BCAR1, RAB25, PRTN3, LSR, SLC11A1, NLRP12, HMBS, LILRB3, GP9, MX2, RASGRP4, ATG16L2 and combinations thereof. In some embodiments, the one or more NDR comprises one or more NDR of a gene selected from the group consisting of: SLC11A1, NLRP12, PRTN3, HMBS, LILRB3, ACSL1, GP9, MX2, RASGRP4, ATG16L2 and combinations thereof. In some embodiments, the one or more NDR comprises one or more NDR of a gene selected from the group consisting of: SHKBP1, ACSL1, BCAR1, RAB25, PRTN3, LSR and combinations thereof.

In various embodiments, where the method comprises a method of estimating a ctDNA burden or tumor burden associated with a specific cancer type or a specific group of cancers, the NDR comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ABTB1, ACAP1, ACO1, ACSL1, ADIRF, AGR2, AGR3, AK2, AKNA, ALDH18A1, ANKS4B, ARAP1, ARHGAP25, ARHGAP30, ARHGAP9, ARHGEF16, ARHGEF35, ARIDSA, ARRB2, ARSE, ATG16L2, ATP2A2, ATP2C2, ATP5G1, ATP5G3, AXIN2, AZGP1, B3GNT3, BATF2, BCAR1, BCL2L15, BCL2L2, BCL6, BDH1, BDH2, BEST1, BGN, BIN2, BIRCS, BMP4, BOK, BSPRY, C10orf54, C11orf21, C16orf54, C19orf33, C1orf162, C1orf210, C1QTNF5, C3, C5AR2, C6orf203, C8orf59, CA1, CALD1, CAMP, CAPNS, CCL20, CCM2, CCR7, CD177, CD276, CD300LF, CD37, CD44, CD55, CDC42SE1, CDCP1, CDH1, CDH17, CDHRS, CDK4, CDK5RAP2, CDX1, CEACAM1, CEACAM4, CEACAM5, CENPF, CFD, CFTR, CHCHD6, CHID1, CKB, CKMT1B, CLDN7, CLEC4E, CORO1A, COTL1, COX6C, CR1, CRB3, CSF3R, CTGF, CTNND1, CXCR1, CXCR2, DDC, DDR1, DDX10, DENND1C, DMTN, DOK3, DPEP2, ECT2, EEF1E1, EFNA3, E124, ELF3, EMP1, ENTPD2, ENTPD6, EPCAM, EPHA2, EPS8L3, ERBB2, ERBB3, EVI2B, F3, FAM101A, FAM109A, FAM212B, FAM213B, FAM60A, FAM65B, FAM83E, FAM84A, FBL, FBXL5, FCAR, FCGR2A, FCN1, FERMT1, FFAR2, FGD3, FGFR4, FGFRL1, FGR, FMNL1, FN1, FOLR3, FOXA2, FPR1, FUT2, FUT6, GATA1, GBP3, GGT6, GJB2, GLRX3, GLT1D1, GMFG, GMNN, GNLY, GOLT1A, GPC4, GPR35, GPRCSA, GPX2, GRTP1, GZMH, H2AFY2, HBB, HBD, HBG2, HBM, HBQ1, HCK, HID1, HK3, HKDC1, HMGB3, HMGCS2, HN1L, HNF4A, HTRA1, ICAM3, IFITM1, IFITM2, IFT172, IKZF1, IL1R2, IL1 RN, IL32, ILVBL, IPO5, ITGA2B, ITGAM, ITGB4, JUND, JUP, KIAA1191, KIFC1, LAD1, LAMB2, LAMC2, LDHA, LGALS3BP, LGALS4, LGMN, LILRB2, LILRB3, LIMD2, LIPH, LMNB1, LMO7, LRRC25, LSR, LST1, MACROD1, MAGED1, MAP1LC3A, MEP1A, MFAP4, MIS18A, MISP, MKNK1, MMAB, MME, MMP11, MMP25, MMP7, MMP8, MORN2, MPP1, MPZL2, MRPL17, MSRB1, MTIF2, MUC13, MXD3, MYL4, MYO1A, MYO1F, MZT2A, NAMPT, NCF1, NCF2, NCF4, NDUFAF4, NDUFB5, NEK2, NFAM1, NFE2, NKG7, NNMT, NOX1, NQO1, NTMT1, NTPCR, NUPR1, OAZ1, ORM1, OSCAR, P2RX1, PADI4, PALLD, PDHA1, PDHX, PGLYRP1, PHC2, PHGR1, PHOSPHO1, PIK3R5, PIN4, PLCD3, PLEKHA1, PLS3, POF1B, POLR2H, POSTN, PPIL1, PPM1M, PPP1R16A, PPP1R1B, PRAM1, PRAP1, PRKCB, PROCR, PRR15, PRR15L, PRRC1, PRSS22, PRSS8, PRTN3, PSTPIP1, PTPN6, PYCR1, RAB25, RARRES2, RASGRP2, RASGRP4, RASSF2, REG4, RETN, RFC3, RHOD, RHPN2, RIN3, RNASE4, RND3, RPL41, RPS6KA1, RPS9, S100A12, S100A14, S100A8, S1PR4, SCNN1A, SCOC, SDCBP2, SEC11C, SEPP1, SEPTIN10, SFN, SHKBP1, SIGLEC5, SIRPB1, SLC11A1, SLC25A37, SLC2A8, SLC39A5, SMIM22, SNCA, SORD, SPATS2L, SPI1, SRC, STAP2, STARD10, STEAP1, STX11, SULT2B1, SYTL3, TBC1D10C, TCEAL4, TFF3, THBS2, THEMIS2, TIMM8B, TINAGL1, TJP3, TM4SF5, TMBIM6, TMC4, TMEM106C, TMEM126B, TMEM14A, TMEM14C, TMEM71, TMEM91, TMEM97, TMPRSS2, TMPRSS4, TNFRSF10C, TNS3, TOP2A, TPM1, TRAF3IP3, TREM1, TRIM22, TSKU, TSPAN15, TSPAN8, TYMS, TYROBP, UBE2C, UBE2T, UGT8, UQCC2, URI1, USH1C, VARS, VAV1, VMP1, VNN2, VPS51, VSTM1, VWA1, WAS, WDR12, WFS1, XPO6, ZAP70, ZDHHC19, ZDHHC9, ZNF467 and ZWINT.

In some embodiments, where the method comprises a method of estimating a ctDNA burden or tumor burden associated with a specific cancer type or a specific group of cancers, the NDR comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ACAP1, ACSL1, ADIRF, ANKS4B, ARHGAP30, ARSE, ATP5G3, BCAR1, BCL6, BGN, BIN2, BMP4, C19orf33, C1orf162, C5AR2, CCR7, CD276, CD37, CD44, CDC42SE1, CDH17, CDK5RAP2, CHCHD6, CKB, CLDN7, CLEC4E, CTGF, DDX10, ELF3, ERBB3, F3, FAM101A, FAM65B, FAM84A, FBXL5, FCAR, FCN1, FERMT1, FFAR2, FOLR3, FOXA2, FUT2, GMFG, GPRC5A, GPX2, HBB, HBD, HID1, LAMC2, LDHA, LGALS4, LGMN, LIMD2, LRRC25, LSR, MAGED1, MPZL2, MRPL17, MXD3, MYO1A, NCF1, NCF2, NFE2, OAZ1, PHOSPHO1, PLCD3, POF1B, POSTN, PPP1R16A, PRAP1, PRR15L, PRSS8, PRTN3, RAB25, RASGRP4, RFC3, S100A12, SCOC, SDCBP2, SEPP1, SHKBP1, SLC11A1, SORD, SRC, STAP2, STARD10, STX11, SYTL3, TCEAL4, TFF3, TM4SF5, TMC4, TMEM126B, TMPRSS2, TNFRSF10C, TRAF3IP3, TREM1, TRIM22, TYMS, TYROBP, UBE2C, UGT8, UQCC2, VAV1, VNN2, WAS and ZDHHC9.

In some embodiments, where the method comprises a method of estimating a ctDNA burden or tumor burden associated with a specific cancer type or a specific group of cancers, the NDR comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ACSL1, ANKS4B, ARHGAP30, ATP5G3, B3GNT3, BCL6, BIN2, BMP4, C19orf33, C1orf162, CD37, CLEC4E, ERBB3, FBXL5, FCAR, FCN1, FERMT1, FFAR2, FOXA2, GMFG, HBB, HID1, ICAM3, LGALS4, LGMN, LSR, MXD3, MYO1A, NCF1, NCF2, NFE2, OAZ1, PHOSPHO1, PLCD3, PRAP1, PRSS8, PRTN3, RAB25, RASGRP4, SCOC, SDCBP2, SEPP1, SHKBP1, SYTL3, TFF3, TM4SF5, TMC4, TMPRSS2, TRAF3IP3, TRIM22, TYROBP, UGT8, VAV1 and WAS.

In some embodiments, where the method comprises a method of estimating a ctDNA burden or tumor burden associated with a specific cancer type or a specific group of cancers, the NDR comprises the following: first exon-intron junction of SHKBP1, first exon-intron junction of ACSL1, first exon-intron junction of BCAR1, promoter of RAB25, promoter of PRTN3 and/or promoter of LSR.

In some embodiments, the method further comprises assigning the most weight to the level of cfDNA that maps to the first exon-intron junction of SHKBP1 and less weight to the level of cfDNA that maps to the other NDR(s) when estimating the ctDNA burden. In some embodiments, the method comprises assigning more weight to the level of cfDNA that maps to the first exon-intron junction of SHKBP1 and relatively less weight to the level of cfDNA that maps to one or more of the following NDRs: the first exon-intron junction of ACSL1, the first exon-intron junction of BCAR1, the promoter of RAB25, the promoter of LSR and the promoter of PRTN3. In some embodiments, the method comprises assigning more weight to the level of cfDNA that maps to the first exon-intron junction of ACSL1 and relatively less weight to the level of cfDNA that maps to one or more of the following NDRs: the first exon-intron junction of BCAR1, the promoter of RAB25, the promoter of LSR and the promoter of PRTN3. In some embodiments, the method comprises assigning more weight to the level of cfDNA that maps to the first exon-intron junction of BCAR1 and relatively less weight to the level of cfDNA that maps to one or more of the following NDRs: the promoter of RAB25, the promoter of LSR and the promoter of PRTN3. In some embodiments, the method comprises assigning more weight to the level of cfDNA that maps to the promoter of RAB25 and relatively less weight to the level of cfDNA that maps to one or more of the following NDRs: the promoter of LSR and the promoter of PRTN3. In various embodiments, the method comprises assigning more weight to the level of cfDNA that maps to the promoter of LSR and relatively less weight to the level of cfDNA that maps to the promoter of PRTN3. In some embodiments, the method comprises assigning more weight to the level of cfDNA that maps to a first exon-intron junction and relatively less weight to the level of cfDNA that maps to a promoter.

In various embodiments, the specific cancer type or specific group of cancers comprises colorectal cancer.

In various embodiments, where the method comprises a method of estimating a ctDNA burden or tumor burden associated with any cancer or cancer in general (pan cancer), the NDR comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ABHD5, ABTB1, ACAP1, ACRBP, ACSL1, ADAM8, AHSP, AKNA, ALAS2, ALOX5, ANPEP, AOAH, APOBEC3A, ARAP1, ARHGAP26, ARHGAP9, ARID5A, ARRB2, ATG16L2, ATP6V1B2, AZU1, BIN2, BMX, BPI, BTK, BTNL8, C11orf21, C19orf35, C1orf162, C1orf228, C6orf25, CA1, CA4, CAMP, CARS2, CCDC88B, CCND3, CD177, CD244, CD300E, CD300LB, CD37, CD44, CD53, CDK5RAP2, CEACAM3, CEACAM4, CELF2, CFP, CLC, CLEC12A, CLEC4D, CLEC4E, CMTM2, CNN2, CORO1A, CORO7, CR1, CSF3R, CXCR1, CXCR2, CYBA, CYTH4, DEF8, DEFA1, DEFA1B, DEFA3, DEFA4, DENND1C, DENND3, DHRS13, DHX34, DMTN, DNAH17, DOCK2, DOK3, DYSF, ECE1, EGLN2, ELANE, EPB42, FAM49B, FAM65B, FBXL5, FCAR, FCGR2A, FCGR3B, FCN1, FERMT3, FES, FFAR2, FGD3, FGR, FKBP8, FLOT2, FMNL1, FOLR3, FPR2, FUT7, GATA1, GCA, GNG2, GNLY, GP9, GPSM3, GRAMD1A, GRAP2, HBA1, HBA2, HBB, HBD, HBG1, HBG2, HBM, HBQ1, HCK, HCLS1, HK3, HMBS, ICAM3, IFI30, IFITM1, IFITM2, IL16, IL18RAP, IL1R2, IL2RG, IMPDH1, INPP5D, ITGA2B, ITGAL, ITGAM, ITGAX, ITGB2, JAK3, KCNAB2, KCNE1, KLF1, LAPTM5, LCP2, LILRA1, LILRA5, LILRB2, LILRB3, LPAR2, LRCH4, LSP1, LST1, LTB, LYL1, MAN2A2, MEFV, MKNK1, MLKL, MMP25, MMP8, MPO, MPP1, MSL3, MSRB1, MX2, MXD3, MYL4, MYO1F, MYO1G, NABP1, NADK, NAIP, NAMPT, NARF, NBEAL2, NCF1, NCF2, NDEL1, NFE2, NLRC4, NLRC5, NLRP12, P2RX1, PADI2, PADI4, PARVG, PGD, PGLYRP1, PHF21A, PHOSPHO1, PIK3R5, PLB1, PLBD1, PLCB2, PLCG2, PPBP, PRAM1, PREX1, PROK2, PRTN3, PSTPIP1, PTK2B, PTPN6, PYGL, R3HDM4, RAB24, RAC2, RASAL3, RASGRP2, RASGRP4, RELT, REM2, RGL4, RGS19, RIN3, RNF166, RNF167, SAP25, SASH3, SECTM1, SELL, SEMA4D, SHKBP1, SIGLEC5, SIPA1, SIRPB1, SLC11A1, SLC12A9, SLC16A3, SLC2A3, SLC38A5, SLC43A2, SLCO3A1, SMAP2, SORL1, SPI1, ST20, STAT5B, STK10, STXBP2, TACC3, TAGAP, TALDO1, TBC1D10C, TBX21, TBXAS1, THEMIS2, TLR6, TMCC2, TMEM71, TNFRSF10C, TRAF3IP3, TREML2, TRIM25, TUBA4A, TUBB1, UBE2D3, UNC13D, USB1, VASP, VAV1, VMP1, VNN3, VSTM1, WAS, XPO6, ZAP70, ZDHHC18 and ZDHHC19.

In some embodiments, where the method comprises a method of estimating a ctDNA burden or tumor burden associated with any cancer or cancer in general (pan cancer), the NDR comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ABTB1, ACAP1, ACSL1, ARHGAP9, ATG16L2, ATP6V1B2, BIN2, BTK, BTNL8, C19orf35, CA4, CD37, CDK5RAP2, CEACAM4, CFP, CLEC12A, CLEC4D, CLEC4E, CSF3R, CXCR2, CYTH4, DEF8, DENND1C, DENND3, DHRS13, DOK3, FAM49B, FBXL5, FCGR2A, FCN1, FES, FFAR2, FKBP8, FMNL1, FOLR3, FUT7, GNG2, GP9, GPSM3, HBD, HK3, HMBS, IFI30, IL16, IL1R2, ITGA2B, JAK3, KCNE1, LCP2, LILRB2, LILRB3, LYL1, MAN2A2, MKNK1, MLKL, MPO, MX2, MYO1F, NCF1, NFE2, NLRP12, PADI2, PADI4, PARVG, PGLYRP1, PHOSPHO1, PREX1, PRTN3, PSTPIP1, RAC2, RASAL3, RASGRP4, RELT, RNF166, RNF167, SHKBP1, SLC11A1, SLC12A9, SLC16A3, SLCO3A1, SORL1, SPI1, TBC1D10C, TBXAS1, USB1, VAV1, VSTM1, XPO6 and ZDHHC18.

In some embodiments, where the method comprises a method of estimating a ctDNA burden or tumor burden associated with any cancer or cancer in general (pan cancer), the NDR comprises one or more, or at least about one, at least about two, at least about three, at least about four, at least about five, at least about six, at least about seven, at least about eight, at least about nine, or at least about ten of the following genes/associated NDRs (e.g. a transcription start site, a promoter, an intron-exon junction and/or an exon-intron junction): ABTB1, ACAP1, ACSL1, ATG16L2, ATP6V1B2, BTK, BTNL8, C19orf35, CEACAM4, CLEC4E, CSF3R, DENND1C, DENND3, DHRS13, FBXL5, FCAR, FCN1, FFAR2, FKBP8, FMNL1, GNG2, GP9, GPSM3, HBD, HMBS, IFI30, IL18RAP, ITGA2B, LCP2, LILRB3, LYL1, MAN2A2, MKNK1, MPO, MX2, MXD3, MYO1F, NFE2, NLRP12, PADI4, PHOSPHO1, PREX1, RASGRP2, RASGRP4, RGL4, RNF166, RNF167, SHKBP1, SLC11A1, SLC12A9, TBC1 D10C, TBXAS1, USB1 and VSTM1.

In some embodiments, where the method comprises a method of estimating a ctDNA burden or tumor burden with any cancer or cancer in general (pan cancer), the NDR comprises the following: promoter of SLC11A1, promoter of NLRP12, promoter of PRTN3, promoter of HMBS, promoter of LILRB3, first exon-intron junction of ACSL1, first exon-intron junction of GP9, promoter of MX2, promoter of RASGRP4 and/or promoter of ATG16L2.

In some embodiments, the method further comprises assigning the most weight to the level of cfDNA that maps to the promoter of HMBS and/or the first exon-intron junction of GP9 and relatively less weight to the level of cfDNA that maps to the other NDR(s) when estimating the ctDNA burden. In some embodiments, the method comprises assigning more weight to the level of cfDNA that maps to the promoter of HMBS and/or the first exon-intron junction of GP9 and relatively less weight to the level of cfDNA that maps to one or more of the following NDRs: the promoter of RASGRP4, the promoter of NLRP12, the promoter of ATG16L2, the promoter of SLC11A1, the promoter of LILRB3, the promoter of PRTN3, the promoter of MX2 and the first exon-intron junction of ACSL1. In some embodiments, the method comprises assigning more weight to the level of cfDNA that maps to the promoter of RASGRP4 and relatively less weight to the level of cfDNA that maps to one or more of the following NDRs: the promoter of NLRP12, the promoter of ATG16L2, the promoter of SLC11A1, the promoter of LILRB3, the promoter of PRTN3, the promoter of MX2 and the first exon-intron junction of ACSL1. In some embodiments, the method comprises assigning more weight to the level of cfDNA that maps to the promoter of NLRP12 and relatively less weight to the level of cfDNA that maps to one or more of the following NDRs: the promoter of ATG16L2, the promoter of SLC11A1, the promoter of LILRB3, the promoter of PRTN3, the promoter of MX2 and the first exon-intron junction of ACSL1. In some embodiments, the method comprises assigning more weight to the level of cfDNA that maps to the promoter of ATG16L2 and relatively less weight to the level of cfDNA that maps to one or more of the following NDRs: the promoter of SLC11A1, the promoter of LILRB3, the promoter of PRTN3, the promoter of MX2 and the first exon-intron junction of ACSL1. In some embodiments, the method comprises assigning more weight to the level of cfDNA that maps to the promoter of SLC11A1 and relatively less weight to the level of cfDNA that maps to one or more of the following NDRs: the promoter of LILRB3, the promoter of PRTN3, the promoter of MX2 and the first exon-intron junction of ACSL1. In some embodiments, the method comprises assigning more weight to the level of cfDNA that maps to the promoter of LILRB3 and relatively less weight to the level of cfDNA that maps to one or more of the following NDRs: the promoter of PRTN3, the promoter of MX2 and the first exon-intron junction of ACSL1. In some embodiments, the method comprises assigning more weight to the level of cfDNA that maps to the promoter of PRTN3 and relatively less weight to the level of cfDNA that maps to one or more of the following NDRs: the promoter of MX2 and the first exon-intron junction of ACSL1. In some embodiments, the method comprises assigning similar weights to the level of cfDNA that maps to the promoter of HMBS and the level of cfDNA that maps to the first exon-intron junction of GP9. In some embodiments, the method comprises assigning similar weights to the level of cfDNA that maps to the promoter of MX2 and the level of cfDNA that maps to the first exon-intron junction of ACSL1.

In various embodiments, the total length/size of the one or more NDR is no more than about 100 kilobase pairs (kb), no more than about 90 kb, no more than about 80 kb, no more than about 70 kb, no more than about 60 kb, no more than about 50 kb, no more than about 30 kb, no more than about 20 kb or more than about 10 kb. In some embodiments, the total length/size of the one or more NDR is no more than about 30 kb. In some embodiments, the total length/size of the one or more NDR is no more than about 25 kb. In some embodiments, the total length/size of the one or more NDR is about 24 kb.

In various embodiments, the method does not comprise sequencing one or more regions that collectively spans more than about 100 kb, more than about 95 kb, more than about 90 kb, more than about 85 kb, more than about 80 kb, more than about 75 kb, more than about 70 kb, more than about 65 kb, more than about 60 kb, more than about 55 kb, more than about 50 kb, more than about 45 kb, more than about 40 kb, more than about 35 kb, more than about 30 kb, more than about 25 kb, more than about 20 kb, more than about 15 kb, more than about 10 kb or more than about 5 kb in length. In various embodiments, the method does not comprise sequencing a continuous/contiguous region that spans more than about 4 kb, more than about 5 kb, more than about 6 kb, more than about 7 kb, more than about 8 kb, more than about 9 kb or more than about 10 kb in length. In various embodiments, the method does not comprise whole genome sequencing of the cfDNA. Advantageously, embodiments of the method are efficient in terms of time and resources, and provide a fast turnaround time.

As may be appreciated, by following the teachings herein/carrying out the steps of this disclosure, a person skilled in the art will also be able to identify further genomic regions (including non-coding regions), other than the ones highlighted in this disclosure, that are also predictive of the disease burden, the cancer burden, the tumor burden, the ctDNA burden, the level of ctDNA, an amount of ctDNA, the proportion of ctDNA, the fraction of ctDNA and the ctDNA content. Hence, the genes/regions highlighted in this disclosure are non-exhaustive. Indeed, a person skilled in the art would understand that the genomic regions that may be used to predict the disease burden, the cancer burden, the tumor burden, the ctDNA burden, the level of ctDNA, an amount of ctDNA, the proportion of ctDNA, the fraction of ctDNA and/or the ctDNA content are not limited to the particular gene-encoding regions described herein, and may also include non-coding regions (including regions that are far away from genes e.g. regulatory regions such as enhancers).

In various embodiments, the method further comprises removing particulate blood components from the sample (e.g. a blood sample) to leave behind blood plasma for use in the determining step. In various embodiments, plasma is separated from blood shortly after (e.g. within about 2 hours of) venipuncture. In various embodiments, plasma is separated from blood by centrifugation e.g. at 10 min×300 g and 10 min×9370 g). In various embodiments, the plasma is stored at low temperature e.g. at −80° C. after separation. In various embodiments, the particulate blood components are selected from the group consisting red blood cells, white blood cells, platelets and combinations thereof. In various embodiments, the method further comprising extracting/isolating/purifying the cfDNA from the sample/blood plasma.

In various embodiments, the method requires no more than about 20 milliliters, no more than about 19.5 milliliters, no more than about 19 milliliters, no more than about 18.5 milliliters, no more than about 18 milliliters, no more than about 17.5 milliliters, no more than about 17 milliliters, no more than about 16.5 milliliters, no more than about 16 milliliters, no more than about 15.5 milliliters, no more than about 15 milliliters, no more than about 14.5 milliliters, no more than about 14 milliliters, no more than about 13.5 milliliters, no more than about 13 milliliters, no more than about 12.5 milliliters, no more than about 12 milliliters, no more than about 11.5 milliliters, no more than about 11 milliliters, no more than about 10.5 milliliters, no more than about 10 milliliters, no more than about 9.5 milliliters, no more than about 9 milliliters, no more than about 8.5 milliliters, no more than about 8 milliliters, no more than about 7.5 milliliters, no more than about 7 milliliters, no more than about 6.5 milliliters, no more than about 6 milliliters, no more than about 5.5 milliliters, no more than about 5 milliliters, no more than about 4.5 milliliters, no more than about 4 milliliters, no more than about 3.5 milliliters, no more than about 3 milliliters, no more than about 2.5 milliliters, no more than about 2 milliliters, no more than about 1.5 milliliters, no more than about 1 milliliters, no more than about 0.9 milliliters, no more than about 0.8 milliliters, no more than about 0.7 milliliters, no more than about 0.6 milliliters, no more than about 500 microliters, no more than about 450 microliters, no more than about 400 microliters, no more than about 350 microliters or no more than about 300 microliters of sample.

In various embodiments, the method further comprises obtaining the sample from the subject prior to the determining step. In various embodiments, the step of obtaining the sample from the subject is a non-surgical step, a non-invasive step or a minimally invasive step. In various embodiments, the step of obtaining the sample from the subject comprises withdrawing a blood sample from the subject.

In various embodiments, the method is capable of precisely estimating one or more of: a disease burden, a cancer burden, a tumor burden, a ctDNA burden, a level of ctDNA, an amount of ctDNA, a proportion of ctDNA, a fraction of ctDNA and a ctDNA content such that the estimated disease burden, cancer burden, tumor burden, ctDNA burden, level of ctDNA, amount of ctDNA, proportion of ctDNA, fraction of ctDNA and/or ctDNA content has an absolute deviation/absolute error/mean absolute deviation/mean absolute error of no more than about 5.0%, no more than about 4.9%, no more than about 4.8%, no more than about 4.7%, no more than about 4.6%, no more than about 4.5%, no more than about 4.4%, no more than about 4.3%, no more than about 4.2%, no more than about 4.1%, no more than about 4.0%, no more than about 3.9%, no more than about 3.8%, no more than about 3.7%, no more than about 3.6%, no more than about 3.5%, no more than about 3.4%, no more than about 3.3%, no more than about 3.2%, no more than about 3.1%, no more than about 3%, no more than about 2.9%, no more than about 2.8%, no more than about 2.7%, no more than about 2.6%, no more than about 2.5%, no more than about 2.4%, no more than about 2.3%, no more than about 2.2%, no more than about 2.1%, no more than about 2%, no more than about 1.9%, no more than about 1.8%, no more than about 1.7%, no more than about 1.6%, or no more than about 1.5% from a true/expected/measured disease burden, cancer burden, tumor burden, ctDNA burden, level of ctDNA, amount of ctDNA, proportion of ctDNA, fraction of ctDNA and/or ctDNA content.

In various embodiments, the method has a predictive accuracy of at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or at least about 100%.

In various embodiments, the method comprises a machine learning-based method.

In various embodiments, the method further comprises training a machine learning model with a first training data set defining a level (or an amount, a proportion, a fraction and/or a content) of DNA, optionally cfDNA, that maps to (or aligns with, corresponds to, belongs to, is similar to and/or is identical to) the one or more NDR (e.g. in the form of a read depth coverage) as features and a measured disease burden/tumor burden/ctDNA burden/ctDNA level/ctDNA amount/ctDNA proportion/ctDNA fraction/ctDNA content; and selecting a first set of one or more features that is predictive of the disease burden/tumor burden/ctDNA burden/ctDNA level/ctDNA amount/ctDNA proportion/ctDNA fraction/ctDNA content.

In various embodiments, a subset of samples may be randomly selected from a training data set to train the machine learning model to identify the most predictive features. In various embodiments, the foregoing may be repeated independently multiple times, e.g. 1000 times, and the time(s) each feature is chosen as a predictive feature is counted. In various embodiments, the feature(s) that is/are selected most frequently is/are extracted to train a final model comprising all samples in the training data set (e.g. the first training data set) to identify one or more features (e.g. the first set of one or more features) that is predictive of the disease burden/tumor burden/ctDNA burden/ctDNA level/ctDNA amount/ctDNA proportion/ctDNA fraction/ctDNA content. In various embodiments, cross validation (e.g. five-fold cross validation, eight-fold cross validation, ten-fold cross validation) is carried out during the machine learning process for identifying the most predictive features.

In various embodiments, the selecting step further comprises employing a linear model/regression, optionally a sparse linear model/regression, further optionally a Lasso (least square absolute shrinkage and selection operator) model to identify the first set of one or more features that is predictive of the disease burden/tumor burden/ctDNA burden/ctDNA level/ctDNA amount/ctDNA proportion/ctDNA fraction/ctDNA content.

In various embodiments, the method further comprises providing a test data set to the trained machine learning model, the test data set defining at least the first set of one or more selected features; and estimating the disease burden/tumor burden/ctDNA burden/ctDNA level/ctDNA amount/ctDNA proportion/ctDNA fraction/ctDNA content based on at least the first set of one or more selected features.

In various embodiments, the method further comprises comparing the estimated disease burden/tumor burden/ctDNA burden/ctDNA level/ctDNA amount/ctDNA proportion/ctDNA fraction/ctDNA content with a true/expected/measured disease burden/tumor burden/ctDNA burden/ctDNA level/ctDNA amount/ctDNA proportion/ctDNA fraction/ctDNA content of the test data set; and calculating an absolute deviation/absolute error/mean absolute deviation/mean absolute error between the estimated and the true/expected/measured disease burden/tumor burden/ctDNA burden/ctDNA level/ctDNA amount/ctDNA proportion/ctDNA fraction/ctDNA content to evaluate a performance/prediction accuracy of the model.

In various embodiments, the method further comprises obtaining/collecting blood samples comprising cfDNA from cancer patients and healthy individuals; measuring a level (or an amount, a proportion, a fraction and/or a content) of DNA that maps to (or aligns with, corresponds to, belongs to, is similar to and/or is identical to) the one or more NDR (e.g. in the form of a read depth coverage) to obtain the features of the first training data set; and measuring a disease burden/tumor burden/ctDNA burden/ctDNA level/ctDNA amount/ctDNA proportion/ctDNA fraction/ctDNA content (e.g. by whole genome sequencing, deep whole genome sequencing etc.) to obtain the disease burden/tumor burden/ctDNA burden/ctDNA level/ctDNA amount/ctDNA proportion/ctDNA fraction/ctDNA content of the first training data set. In various embodiments, the method further comprises determining/measuring an expression of the plurality of genes associated with the NDR in the blood samples.

In various embodiments, the method further comprises obtaining/collecting tumor/tumor biopsy samples from cancer patients; extracting/isolating/purifying nucleic acids from the tumor/tumor biopsy samples; measuring from the nucleic acids a level (or an amount, a proportion, a fraction and/or a content) of DNA that maps to (or aligns with, corresponds to, belongs to, is similar to and/or is identical to) the one or more NDR (e.g. in the form of a read depth coverage); and measuring from the nucleic acids an expression of the genes associated with the one or more NDR in the tumor/tumor biopsy samples.

In various embodiments, the method further comprises comparing said level (or an amount, a proportion, a fraction and/or a content) of DNA (e.g. in the form of a read depth coverage) and/or the expression of the genes between the blood samples and the tumor/tumor biopsy samples; identifying genes that show substantially different level (or an amount, a proportion, a fraction and/or a content) of DNA (e.g. substantially different read depth coverages) and/or substantially different expressions between the blood samples and the tumor/tumor biopsy samples; and selecting the level (or an amount, a proportion, a fraction and/or a content) of DNA (e.g. in the form of a read depth coverage) of these identified genes in the blood sample as features to be input in the first training data set.

The method may further comprise removing the first set of one or more features from the first training data set to form a second training data set; and training the machine learning model with the second training data set to select a second set of one or more features that is predictive of the disease burden, the cancer burden, the tumor burden, the ctDNA burden, the level of ctDNA, an amount of ctDNA, the proportion of ctDNA, the fraction of ctDNA and/or the ctDNA content. These steps may be repeated one or more times to obtain a third, fourth, fifth etc. set of one or more features that is predictive of the disease burden, the cancer burden, the tumor burden, the ctDNA burden, the level of ctDNA, an amount of ctDNA, the proportion of ctDNA, the fraction of ctDNA and/or the ctDNA content by removing the second, third, fourth etc. set of one or more features from the second, third, fourth etc. training data set to form a third, fourth, fifth etc. training data set respectively.

In various embodiments, the method further comprises screening for/detecting a tumor-specific mutation in the cfDNA/ctDNA present in the blood sample. Advantageously, embodiments of the method simultaneously allow for profiling of actionable cancer mutations and quantitative estimation of the disease burden, the cancer burden, the tumor burden, the ctDNA burden, the level of ctDNA, an amount of ctDNA, the proportion of ctDNA, the fraction of ctDNA and/or the ctDNA content. The method may be performed in combination with or complimentary to existing sequencing-based methods in cancer detection/monitoring.

In various embodiments, the method is an in vitro or ex vivo method.

In various embodiments, the method is a liquid biopsy method.

In various embodiments, the method is a method of determining disease progression in a subject and the method further comprises: determining in a subsequent blood sample obtained from the subject, a level of cfDNA that maps to one or more NDR; estimating the ctDNA burden or tumor burden based on said level of cfDNA; comparing the ctDNA burden or tumor burden estimated from said subsequent blood sample with the ctDNA burden or tumor burden estimated from said blood sample; and optionally identifying the subject as having disease progression if the ctDNA burden or tumor burden estimated from said subsequent blood sample is higher than the ctDNA burden or tumor burden estimated from said blood sample or identifying otherwise if the ctDNA burden or tumor burden estimated from said subsequent blood sample is not higher than the ctDNA burden or tumor burden estimated from said blood sample. In various embodiments, where the ctDNA burden or tumor burden estimated from said subsequent blood sample is lower than the ctDNA burden or tumor burden estimated from said blood sample, the disease identified to be improving/abating in the subject. In various embodiments, where the ctDNA burden or tumor burden estimated from said subsequent blood sample is substantially the same as the ctDNA burden or tumor burden estimated from said blood sample, the disease is identified to be stable in the subject.

Disease progression in a subject may be indicative of resistance to the current treatment regimen received by the subject. Thus, the method may also be useful for identifying resistance to treatment in a subject. In various embodiments therefore, the method further comprises changing the treatment regimen received by the subject if the subject is identified as having disease progression. Changing the treatment regimen may involve subjecting/exposing the subject to a second therapy that is different from the current or the first therapy. Changing the treatment regimen may involve replacing the current treatment regimen received by the subject with another treatment regimen, or it may involve administering to the subject additional therapies in addition to the current treatment regimen. In some embodiments, where a subject is already receiving combination therapy, changing the treatment regimen may also involve removing one or more therapies from the combination therapy. Examples of treatment regimens/therapies include, but are not limited to, chemotherapy, radiotherapy, gene therapy, hormonal therapy, immunotherapy, surgical therapy, combination therapy, alternative therapy/complementary therapy and combinations thereof. In various embodiments, changing the treatment regimen does not necessarily entail switching from one class of therapy (e.g. one of chemotherapy, radiotherapy, gene therapy, hormonal therapy, immunotherapy, surgical therapy, combination therapy, alternative therapy/complementary therapy and combinations thereof) to another class of therapy, although it may involve such a switch. Changing the treatment regimen may involve changing from one specific therapy to another specific therapy within the same therapy class. For example, changing the treatment regimen may involve changing the particular chemotherapy drug received by the subject.

In various embodiments, there is provided a method of monitoring disease progression in a subject, the method comprising: determining in a first sample comprising cfDNA obtained from the subject at a first time point, a first level (or an amount, a proportion, a fraction and/or a content) of cfDNA that maps to one or more NDR; estimating a first ctDNA burden or tumor burden (or a disease burden, a cancer burden, a level of ctDNA, an amount of ctDNA, a proportion of ctDNA, a fraction of ctDNA and a ctDNA content) in the subject based on the first level of cfDNA, determining in a second sample comprising cfDNA obtained from the subject at a second time point, a second level of DNA that maps to the one or more NDR, estimating a second ctDNA burden or tumor burden based on the second level of cfDNA; and comparing the first and the second estimated ctDNA burden or tumor burden to determine whether the disease has progressed, wherein the second time point is later than the first time point. In various embodiments, where the second estimated ctDNA burden or tumor burden is higher than the first estimated ctDNA burden or tumor burden, the disease is considered to have progressed/worsened. In various embodiments, where the second estimated ctDNA burden or tumor burden is lower than or is substantially the same as the first estimated ctDNA burden or tumor burden, the disease is considered to have abated or stabilized.

In various embodiments, there is provided a method of evaluating treatment efficacy/response in a subject, the method comprising: determining in a first sample comprising cfDNA obtained from the subject before/during a treatment/treatment stage, a first level (or an amount, a proportion, a fraction and/or a content) of cfDNA that maps to one or more NDR; estimating a first ctDNA burden or tumor burden (or a disease burden, a cancer burden, a level of ctDNA, an amount of ctDNA, a proportion of ctDNA, a fraction of ctDNA and a ctDNA content) in the subject based on the first level of cfDNA, determining in a second sample comprising cfDNA obtained from the subject after the treatment/treatment stage, a second level of DNA that maps to the one or more NDR, estimating a second ctDNA burden or tumor burden based on the second level of cfDNA; and comparing the first and the second estimated ctDNA burden or tumor burden to determine whether the treatment is effective/the subject is responding to the treatment. In various embodiments, where the second estimated ctDNA burden or tumor burden is higher than the first estimated ctDNA burden or tumor burden, the treatment is considered to be not effective or the subject is considered to be not responding to the treatment. In various embodiments, where the treatment is considered to be not effective or the subject is considered to be not responding to the treatment, the method further comprises adjusting/altering/stopping/halting/discontinuing the treatment regimen. In various embodiments, where the second estimated ctDNA burden or tumor burden is lower than or substantially the same as the first estimated ctDNA burden or tumor burden, the treatment is considered to be effective or the subject is considered to be responding to the treatment. In various embodiments, where the treatment is considered to be effective or the subject is considered to be responding to the treatment, the method further comprises continuing the treatment regimen.

In various embodiments, there is provided a method of determining a risk of cancer (e.g. a risk of development, predisposition, progression, relapse, recurrence, metastasis, abatement of cancer) in a subject, the method comprising: determining in a blood sample obtained from the subject, a level (or an amount, a proportion, a fraction and/or a content) of cfDNA that maps to one or more NDR, optionally estimating a disease burden (or a cancer burden, a tumor burden, a ctDNA burden, a level of ctDNA, an amount of ctDNA, a proportion of ctDNA, a fraction of ctDNA or a ctDNA content) based on said level of cfDNA, and determining the risk of cancer based on the level of cfDNA that maps to the one or more NDR, or the estimated disease burden. In various embodiments, where the level of cfDNA/the estimated disease burden exceeds a predetermined threshold level, the subject is concluded to have an elevated risk of cancer. In various embodiments, where the level of cfDNA/the estimated disease burden does not exceed the predetermined threshold level, the subject is concluded to have a reduced/low/minimal/no risk of cancer. It will be appreciated that it is within the purview of a person skilled in the art to determine the suitable threshold level. For example, the suitable threshold level may be determined by determining the mean level of cfDNA/the mean estimated disease burden of a healthy population e.g. a population that does not suffer from cancer.

In various embodiments, there is provided a method of treating cancer in a subject, the method comprising: determining in a blood sample obtained from the subject, a level of cfDNA that maps to one or more NDR and estimating a ctDNA burden or tumor burden based on said level of cfDNA. In various embodiments, where the level of cfDNA/the estimated ctDNA burden or tumor burden exceeds a predetermined threshold level, the subject is subjected to treatment selected from the group consisting of chemotherapy, radiotherapy, gene therapy, hormonal therapy, immunotherapy, surgical therapy, combination therapy, alternative therapy/complementary therapy and combinations thereof. It will be appreciated that it is within the purview of a person skilled in the art to determine the suitable threshold level. For example, the suitable threshold level may be determined by determining the mean level of cfDNA/the mean estimated ctDNA burden or tumor burden of a healthy population e.g. a population that does not suffer from cancer.

In various embodiments, there is provided a method of profiling a subject, the method comprising: determining in a blood sample obtained from the subject, a level of cfDNA that maps to one or more NDR and estimating a ctDNA burden or tumor burden based on said level of cfDNA.

In various embodiments, there is provided a kit/panel/probe set/primer set, optionally a kit/panel/probe set/primer set for estimating a tumor burden or ctDNA burden in a subject, the kit/panel/probe set/primer set comprising one or more probe/primer that is capable of hybridizing/binding to one or more NDR, where said NDR (i) comprises the NDR of a gene which transcript is differentially expressed between healthy blood tissue and tumor tissue and/or (ii) is degraded to different extents between healthy blood tissue and blood tissue of a tumor-bearing subject. In various embodiments, the one or more probe/primer is capable of hybridizing/binding to a central genomic region related to the one or more NDR. The size of the central genomic region may be about 1 kb, about 2 kb, about 3 kb, about 4 kb, about 5 kb, about 6 kb, about 7 kb, about 8 kb, about 9 kb or about 10 kb. In one example, a plurality of probes/primers hybridize/bind to an approximately 4 kb region centred at an NDR. The binding sites of a plurality of probes/primers to a central genomic region may be continuous or discontinuous within the central genomic region. In various embodiments, the one or more probe/primer has a sequence that is complementary to a sequence of the one or more NDR or parts thereof. In various embodiments, the one or more probe/primer has a sequence that is complementary to a sequence sharing at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% sequence identity with the one or more NDR or parts thereof. In various embodiments, the one or more probe/primer has a sequence that is complementary to a sequence that differs from the one or more NDR or parts thereof by about one, about two, about three, about four or about five nucleotides/bases. In various embodiments, the one or more probe/primer has a sequence that is complementary to a central genomic region or parts thereof related to the one or more NDR. In various embodiments, the one or more probe/primer has a sequence that is complementary to a sequence sharing at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% sequence identity with the central genomic region or parts thereof. In various embodiments, the one or more probe/primer has a sequence that is complementary to a sequence that differs from the central genomic region or parts thereof or parts thereof by about one, about two, about three, about four or about five nucleotides/bases. In one example, the one or more probe/primer has a sequence that is complementary to an approximately 4 kb region centred at an NDR. A skilled person would be able to determine the suitable conditions that would allow the probe/primer to hybridize to the one or more NDR.

In various embodiments, the one or more NDR comprises one or more NDR of a gene listed in one or more of Table 1, Table 2, Table S3, Table S10, Table S14, Table S15, Table S16, Table S17, Table S18, Table S19, Table S20 and Table S21. In various embodiments, the one or more NDR comprises one or more NDR of a gene selected from the group consisting of: SLC11A1, NLRP12, PRTN3, HMBS, LILRB3, ACSL1, GP9, MX2, RASGRP4, ATG16L2 and combinations thereof. In various embodiments, the one or more NDR comprises one or more NDR of a gene selected from the group consisting of: SHKBP1, ACSL1, BCAR1, RAB25, PRTN3, LSR and combinations thereof.

In various embodiments, the kit/panel or the probe set/primer set further comprises a probe/primer for detecting a tumor-specific mutation.

In various embodiments, the one or more probe/primer comprises from about 50 to about 200 nucleotides/bases, from about 90 to about 150 nucleotides/bases or from about 110 to about 130 nucleotides/bases. In various embodiments, the one or more probe/primer comprises no more than about 200, no more than about 190, no more than about 180, no more than about 170, no more than about 160, no more than about 150, no more than about 140, no more than about 130 or no more than about 120 nucleotides/bases. In various embodiments, the one or more probe/primer comprises at least about 50, at least about 60, at least about 70, at least about 80, at least about 90, at least about 100, at least about 110 or at least about 120 nucleotides/bases. In various embodiments, the one or more probe/primer comprises about 120 nucleotides/bases.

In various embodiments, the one or more probe/primer comprises the sequence of one or more of SEQ ID NO: 1 to SEQ ID NO: 577 (i.e. SEQ ID NO; 1, SEQ ID NO:2, SEQ ID NO: 3, and so forth till SEQ ID NO: 577, see Supplementary Data 3) or a sequence sharing at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% sequence identity thereto. In various embodiments, the sequence sharing at least about 75%, at least about 80%, at least about 85%, at least about 90%, at least about 95%, at least about 99% sequence identity with any one of SEQ ID NO: 1 to SEQ ID NO: 577 is capable of hybridizing/binding to the one or more NDR.

In various embodiments, the kit/panel or the probe set/primer set comprises a plurality of probes/primers.

In various embodiments, the kit/panel/primer set/probe set is for estimating a tumor burden or ctDNA burden associated with cancer, optionally colorectal cancer. In various embodiments, the one or more probe/primer is capable of hybridizing/binding to a genomic region of one or more of the following genes: ARID1A, CCNE1, CDH1, CDK6, CTNNB1, EGFR, ERBB2, KRAS, MUC6, MYC, RHOA, RNF43, SMAD4, TP53 or parts thereof. In various embodiments, the one or more probe/primer has a sequence that is complementary to a genomic region of one or more of ARID1A, CCNE1, CDH1, CDK6, CTNNB1, EGFR, ERBB2, KRAS, MUC6, MYC, RHOA, RNF43, SMAD4, TP53 or parts thereof. In various embodiments, the one or more probe/primer has a sequence that is complementary to a sequence sharing at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% sequence identity with the genomic region of one or more of ARID1A, CCNE1, CDH1, CDK6, CTNNB1, EGFR, ERBB2, KRAS, MUC6, MYC, RHOA, RNF43, SMAD4, TP53 or parts thereof. In various embodiments, the one or more probe/primer has a sequence that is complementary to a sequence that differs from the genomic region of one or more of ARID1A, CCNE1, CDH1, CDK6, CTNNB1, EGFR, ERBB2, KRAS, MUC6, MYC, RHOA, RNF43, SMAD4, TP53 or parts thereof by about one, about two, about three, about four or about five nucleotides.

In various embodiments, the one or more probes/primers cover at least about 80%, at least about 85%, at least about 90%, at least about 95%, at least about 96%, at least about 97%, at least about 98%, at least about 99% or about 100% of the target NDR(s)/genomic region(s). In some embodiments, the one or more probes/primers do not overlap each other i.e. the probes/primer are aligned side-by-side when hybridized/bound to the target NDR(s)/genomic region(s). In some embodiments, there is some degree of overlap among adjacent probes/primers (e.g. an overlap of 10 bp, 30 bp, 50 bp, 70 bp, 90 bp etc.).

The number of probes/primers may vary depending on the number of target NDRs/genomic regions, the length/size of the target NDRs/genomic regions and/or the length/size of the probes/primers etc. Higher probe numbers/density may lead to better sampling, although it can also increase the cost of the method. In various embodiments, the number of probes/primers is in the range of from about 25 to about 50, from about 60 to about 80, from about 90 to about 110, from about 125 to about 150, from about 160 to about 180, from about 190 to about 210, from about 225 to about 250, from about 260 to about 280, from about 290 to about 310, from about 325 to about 350, from about 365 to about 390, from about 405 to about 430, from about 445 to about 470, from about 485 to about 510, from about 525 to about 550, or from about 565 to about 590. In various embodiments, the number of probes/primers is at least about 10, at least about 20, at least about 30, at least about 40, at least about 50, at least about 75, at least about 100, at least about 125, at least about 150, at least about 175, at least about 200, at least about 225, at least about 250, at least about 275 or at least about 300. In various embodiments, the number of probes/primers is no more than about 400, no more than about 375, no more than about 350, no more than about 325, no more than about 300, no more than about 275, no more than about 250, no more than about 225 or no more than about 200.

In various embodiments, there is provided a method or a product as described herein.

### BRIEF DESCRIPTION OF FIGURES

FIG. 1 Overview of approach. Deep cfDNA WGS profiles of plasma samples from healthy individuals and cancer patients were compared to identify nucleosome depleted regions (NDRs) with tumor/blood tissue-specific expression and differential cfDNA coverage. A model was trained to predict ctDNA levels from NDR cfDNA coverage. A compact assay targeting predictive NDRs was used to perform longitudinal profiling of ctDNA levels and dynamics.

FIG. 2 Characteristics of cfDNA degradation patterns at promoters and exon-intron junctions. (a) Systematic analysis of gene regulatory regions for association of gene expression and cfDNA relative coverage. Relative coverage refers to cfDNA coverage across the given region when normalized to +/−1 kb flanking regions. The nucleosome depleted regions of promoter (NDR, −150 to 50 bp relative to TSS) and first exon-intron junction (NDR, −300 to −100 bp relative to first exon end) are highlighted. (b) Relative cfDNA coverage of promoter and junction NDRs for expressed (fpkm≥30 in whole blood) and unexpressed genes. (c) Distribution of promoter and junction NDR relative coverage for expressed and unexpressed genes.

FIG. 3 Quantitative estimation of colorectal cancer ctDNA burden. (a) cfDNA relative coverage for the promoter region of PPP1R16A (ENST00000528430) overexpressed in CRC tumors relative to whole blood, and cfDNA relative coverage for the junction region of GMFG (ENST00000602185) overexpressed in whole blood relative to CRC tumors. The grey curve shows the mean coverage across CRC samples. (b) Relative coverage score (see Methods) of NDRs in transcripts differentially expressed between CRC tumors and whole blood. (c) Schematic showing how the predictive model of ctDNA fractions was developed: Differentially expressed genes in CRC and blood were identified, NDR relative coverage features were obtained from in silico generated cfDNA samples, predictive features were selected, and a quantitative model was fitted. (d, e) Comparison of expected (in silico simulation) and observed ctDNA fractions across the CRC cfDNA samples in the d) training and e) test set, respectively. The mean absolute error (MAE) is listed for each sample. (f) Comparison between observed and expected ctDNA fractions in the test set.

FIG. 4 Targeted NDR assay to quantify ctDNA burden and monitor cancer progression. (a) Schematic showing how targeted NDR sequencing, low-pass WGS, and targeted gene sequencing was performed on a cohort of 53 CRC plasma samples. (b) Comparison of ctDNA fractions inferred by targeted NDR-sequencing and low-pass WGS (ichorCNA). (c) Comparison of ctDNA fractions inferred by targeted NDR-sequencing and maximum VAFs (maximum VAF of all SNVs identified in a given plasma sample). (d) NDR-quantified ctDNA burden across serial plasma samples and its association with events of cancer progression treatment response. Somatic SNV VAFs are highlighted for each timepoint; SNVs detected in at least two timepoints are shown. SNVs undetected with standard filtering criteria at given timepoints are indicated with a dashed line. Treatment types and intervals are highlighted. Events of disease progression as inferred by computerized tomography (CT) scans are shown.

FIG. 5 Estimation of ctDNA burden across two distinct cancer types. (a) cfDNA relative coverage across the promoter region of the blood-specific gene, RASGRP4 (ENST00000615340). Dark grey and light grey curves show the mean of the coverages from plasma samples from CRC and BRCA patients respectively, respectively. (b) Schematic showing how the ctDNA content prediction model across CRC and BRCA samples was developed. (c, d) Comparison of expected (in silico simulation) and observed ctDNA fractions across CRC and BRCA cfDNA samples in the c) training and d) test set, respectively. The mean absolute error is listed for each sample.

FIG. 6A systematic analysis of gene regions for association of gene expression and cfDNA relative coverage. Relative cfDNA coverage (normalized to +/−1-2 kb regions) for sets of genes grouped by expression level in whole blood cells across a) first, b) second, c) third exon-intron junctions, d) first, e) second, f) third intron-exon junctions, as well as g) promoter, and h) transcript end region.

FIG. 7 Correlation between relative coverage of NDRs and epigenetic features. For each candidate covariate/predictor, a linear regression is fitted with relative coverage as the response. The Pearson correlation coefficient (y axis, signed square root of R-squared from regression) is shown for each candidate variable. Whole blood gene expression (fpkm) is binned into 6 bins [unexpressed, 0.01<fpkm≤0.1, 0.1<fpkm≤1, 1<fpkm≤5, 5<fpkm≤30, fpkm≤>30] and fitted as a categorical covariate with the unexpressed group as the reference group. Peak files of epigenetic features [DNase, H3K4me3, H3K36me3, H3K27ac, H3K4me1, H3K9me3 and H3K27me3] from primary T-cells (E034) were obtained from the Roadmap Epigenomics Project. Epigenetic features are fitted as binary covariates with no signal as the reference group. Barplots of the correlation (r, square root of R2 multiplied by the coefficient sign) between each feature and relative coverage for (a) Promoter NDRs and (b) Junction NDRs are shown.

FIG. 8 Transcripts differentially expressed between CRC tumors and whole-blood. CRC (fpkm_{CRC}>20, fpkm_{blood}<0.1, dark grey) and whole-blood (fpkm_{CRC}<0.1, fpkm_{blood}>10, light grey) specific transcripts are identified from expression data from TCGA and GTEx.

FIG. 9 The evolution of predictive error with model complexity. Mean absolute error between expected and predicted ctDNA fractions of CRC samples is estimated as a function of model complexity (number of predictive features). The error bar size is the standard deviation of MAE values from 231 CRC training samples (light grey).

FIG. 10 Model performance on 10 test sets generated using different (withheld) healthy samples from the training sets. Individual normal samples (n=29) in the healthy cohort were evenly split into 2 sets, used to dilute the plasma samples from CRC patients in training (CRC-1 to 8 in Table S1) and test (CRC-9 to 12) sets separately. (a) The correlation (Pearson and Spearman) between the expected and observed ctDNA fractions across the 10 test sets. (b) The mean absolute error (MAE) between the expected and observed ctDNA fractions for the 10 test sets.

FIG. 11 Comparison of expected and ichorCNA-predicted ctDNA fractions across the CRC cfDNA samples. (a) ctDNA fractions across the CRC cfDNA samples. (b) Comparison of expected and ichorCNA-predicted ctDNA fractions.

FIG. 12 Performance of ichorCNA when applied to the samples with low ctDNA burden. 31 out of 120 low-ctDNA samples of CRC were predicted as non-cancerous by ichorCNA, highlighted in black. Grey dashed line indicates ctDNA fraction of 0.

FIG. 13 Predictive error as a function of model complexity for two distinct cancer types. The error bar size is the standard deviation of MAE values from 446 training samples (light grey).

FIG. 14 Comparison of expected and observed ctDNA fractions in test set across two distinct cancer types.

FIG. 15A BRCA model using BRCA tumor-specific NDRs. (a) The list of top BRCA tumor-specific NDRs that were used for ctDNA content prediction. (b, c) Comparison of expected and observed ctDNA fractions across the BRCA cfDNA samples in the test set.

FIG. 16 Comparison of the ctDNA fractions determined by the CRC model and the “CRC+BRCA” model for the CRC samples in the test set.

FIG. 17 Comparison of the observed ctDNA fractions in the 53 original cfDNA samples with capture-based NDR sequencing (mean coverage ˜300×) and their downsampled counterparts (100×, 50×, 25×, and 10×, respectively).

FIG. 18 Genomic regions over promoters (top) and first exon-intron junction (bottom) used to calculate relative coverage. The mean coverage of the up and downstream 2kbp flanks (grey) is used as a “normalization factor” for the region of interest (black).

FIG. 19 Overview of machine learning feature selection, model fitting, and train/test set performance for colorectal cancer.

FIG. 20 Extensive identification of all predictive CRC features/regions.

FIG. 21 Pan-cancer model training: Overview of Machine Learning feature selection, model fitting, and train/test set performance for pan-cancer features.

FIG. 22 Pan-cancer feature combinations: Extensive identification of all predictive pan-cancer features/regions

FIG. 23 Additional CRC and pan-cancer feature combinations: Extensive identification of all predictive feature combinations using in silico samples generated with random subsets of healthy samples

FIG. 24 The flow chart of establishing a machine learning model based on expression-specific DNA degradation patterns to predict ctDNA fractions for potentially clinical use

FIG. 25 The evolution of the error between observed and calculated ctDNA fractions with the number of top features for CRC prediction model

FIG. 26 The evolution of the error between observed and calculated ctDNA fractions with the number of top features for pan-cancer prediction model

### EXAMPLES

Example embodiments of the disclosure will be better understood and readily apparent to one of ordinary skill in the art from the following discussions and if applicable, in conjunction with the figures. It will be appreciated that the example embodiments are illustrative, and that various modifications may be made without deviating from the scope of the invention. Example embodiments are not necessarily mutually exclusive as some may be combined with one or more embodiments to form new exemplary embodiments.

It is shown that the size distribution of cfDNA fragments has a mode of −166 bp, suggesting that nucleosome-bound DNA fragments are protected/preserved during cell death and shed into the circulation. Nucleosome depleted regions (NDRs) are therefore more frequently degraded, yielding a nucleosome-dependent degradation footprint in cfDNA profiles, which can be used to infer tissue of origin. The read depth coverage from sequencing plasma cfDNA is shown to be able to identify nucleosome depletion at a gene's promoter and thus infer gene expression. The coverage of the nucleosome-depleted region at a gene's promoter is negatively correlated with the gene's expression level: a highly expressed gene will tend to have less nucleosome binding across its promoter and therefore lower level of protection and higher levels of DNA degradation. Moreover, plasma cfDNA degradation patterns in cancer patients can be used to infer tumor gene expression.

Here, it is hypothesized that a limited set of tumor or blood-specific NDRs could be used to infer the ctDNA burden (fraction) in the blood circulation of cancer patients. ctDNA burden refers to the relative amount of ctDNA out of all cfDNA molecules in a plasma sample. Using deep cfDNA WGS data from cancer patients and healthy individuals, a quantitative model that infers the ctDNA burden using cfDNA sequencing data from a limited set of NDRs is trained and test. This model is shown to be accurate for plasma samples from both colorectal cancer (CRC) and breast cancer (BRCA) patients (mean absolute error 4.3%), and deployment is explored using a compact targeted sequencing assay for low-cost and quantitative tracking of patient ctDNA dynamics.

The examples demonstrate two components. The first component is a method for estimating ctDNA burden specifically in liquid biopsies from colorectal cancer (CRC) patients. The second component is a method for estimating ctDNA burden in liquid biopsies from any solid tumor (pan-cancer). Both colorectal cancer and pan-cancer models have high prediction accuracy, but the pan-cancer model has the added advantage that it can be applied to any solid tumors.

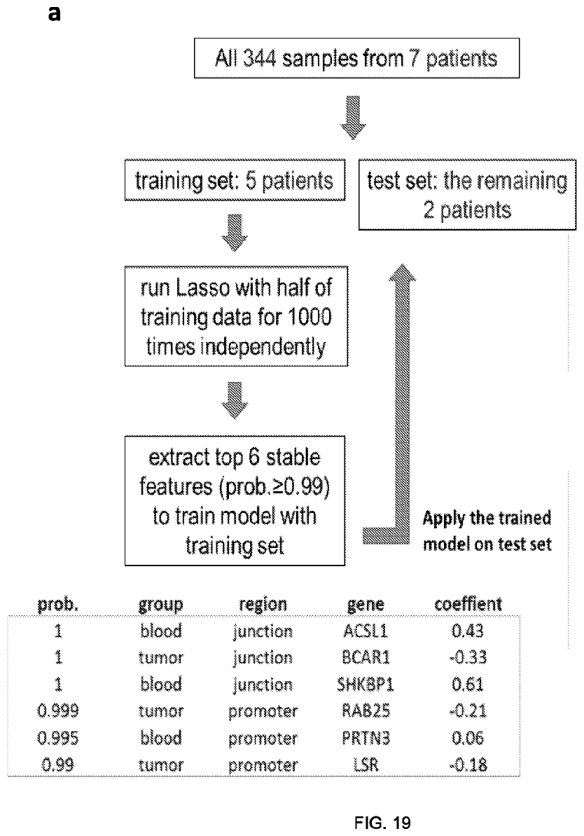

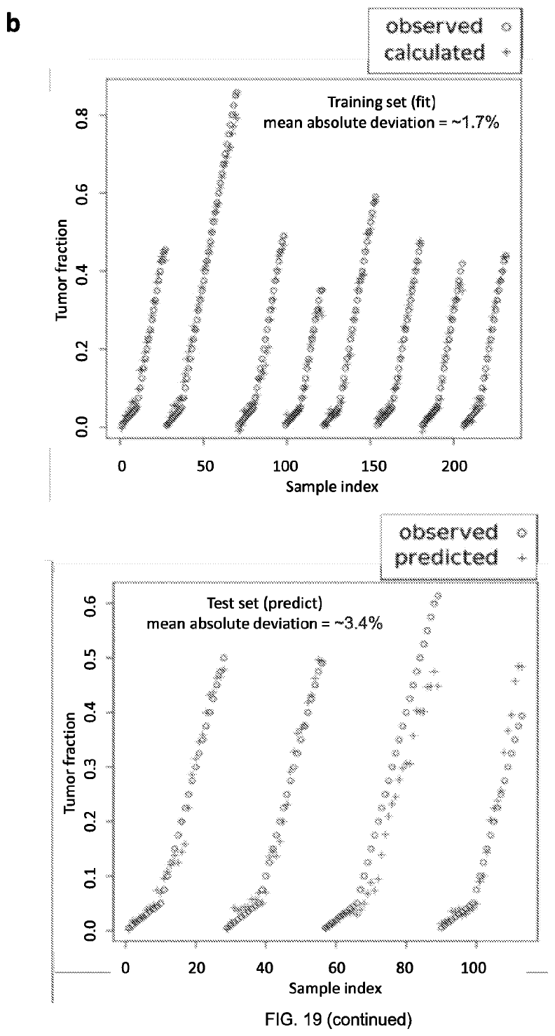

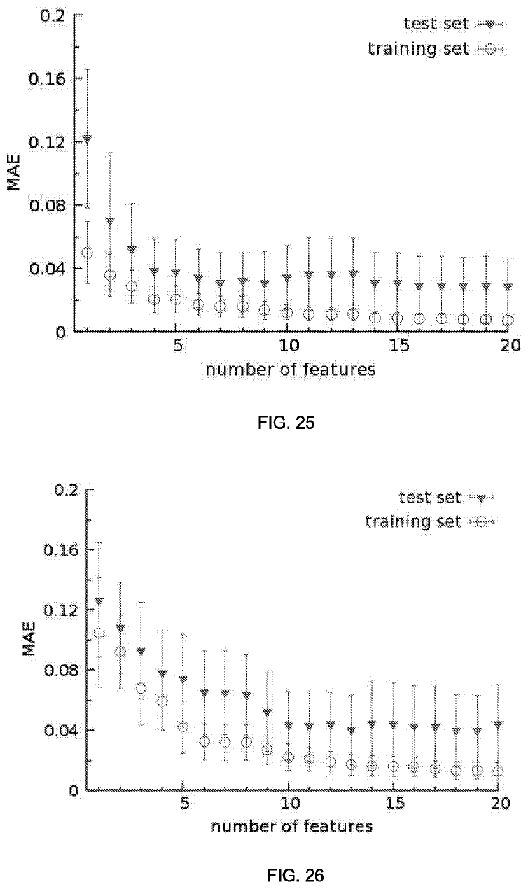

In one example, the colorectal cancer ctDNA burden estimation model is built as follow. Machine learning was used to develop a predictive model that uses cfDNA coverage patterns at the promoter and junction regions of selected genes to infer ctDNA burden in the blood samples of colorectal cancer patients. The model was trained using data from an in silico “dilution” of 8 samples from 5 cancer patients and healthy individuals, resulting in a training set of 231 “virtual” samples of various ctDNA content (see Table S2). The candidate tumor/blood transcripts that showed both differential expression signal and differential DNA degradation signal at NDRs between CRC tumor and blood were shortlisted. The tumor and blood transcripts were pooled together and their promoter and junction NDR coverage scores were defined as (totally 908) input “features” (see Table S3). The coverage value of each position was normalized by the mean coverage of the upstream (−2000 to −1000 bp) and downstream (+1000 to +2000 bp) regions with respect to transcription start site (for promoter) and exon boundary (for junction) respectively. A Lasso (least absolute shrinkage and selection operator) model was employed to identify features predictive of ctDNA proportions. Half of the training data was extracted randomly to run Lasso (using 1000 repetitions), consequently discovering 6 stable features (probability ≥0.99) from this stability-based exploration (FIG. 19). To develop an accurate ctDNA burden estimation model with a minimal sequencing cost, using the 4 to 10 robust features is an optimal solution, because increasing the number of features to over 10 fails in improving the prediction accuracy of test set and thus over-interprets the training set data (see FIG. 25) in one example. Here, the top 6 features were employed as an example to train and test the machine learning model.

The model may also be applicable to other cancer types, subtypes, or specific therapeutic settings, considering tissue-of-origin of cfDNA molecules can be principally informed from tissue-specific DNA degradation pattern. Compared with plasma from healthy people, tumor-derived DNA component in cancer plasma samples weakens the blood-specific DNA degradation pattern, which suggests the decay of blood-specific signal might be informative of robustly estimating the ctDNA content regardless of cancer types. Therefore, the ctDNA content estimation method is also extended to the pan-cancer level.

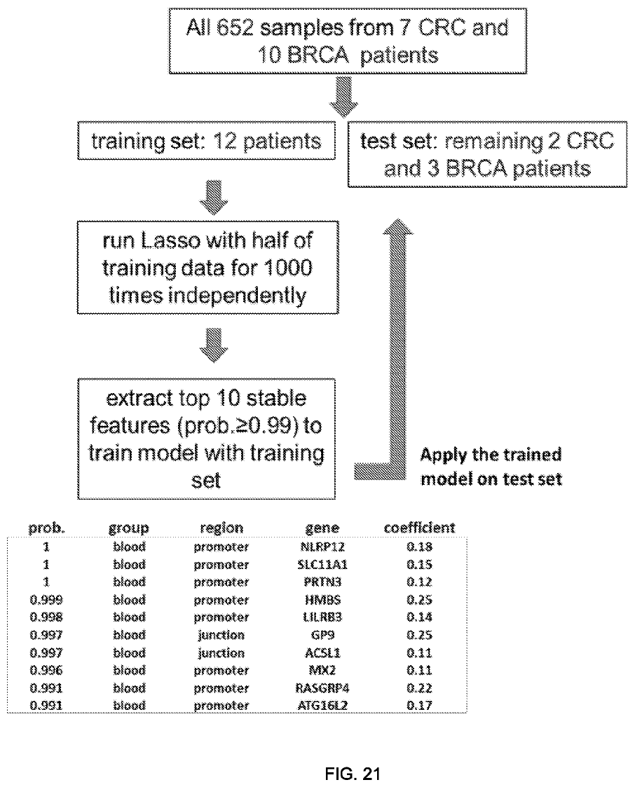

In one example, a pan-cancer ctDNA burden estimation model is built as follows. This pan-cancer model relates to a quantitative method that only uses blood-based features/regions (and no use of tumor type specific regions). First, blood transcripts that are highly expressed in blood and lowly expressed in tumors of all 20 cancer types (BLCA, BRCA, CESC, CRC, ESCA, GBM, HNSC, KIRC, KIRP, LGG, LIHC, LUAD, LUSC, OV, PAAD, PRAD, SKCM, STAD, THCA, and UCEC) where shortlisted. These yielded 792 promoter and junction NDR candidate coverage features (Table S10). 215 in-silico samples diluted from the plasma samples of 7 breast cancer (BRCA) patients were added into the existing training set of colorectal cancer samples, as well as 93 in-silico samples diluted from the plasma samples of 3 BRCA patients into the existing test set (see Table S2). The same protocol of feature selection and model training that were used for colorectal cancer ctDNA burden estimation above was performed. It was found that using around 10 blood features is able to predict the ctDNA content in plasma samples (see FIG. 26). An example of employing 10 stable features (probability≥0.99) (Table 2) that are predictive of ctDNA burden in the training set is shown, with a mean absolute error of ˜2.2% between the calculated and observed ctDNA proportions (FIG. 21). The trained model with those 10 features is able to accurately predict the ctDNA content in the test set (MAE of α4.3%). The prediction on breast cancer cases show a promising prediction power of the pan-cancer model.

Based on the guidance provided by this disclosure, users can follow the methodology details to reproduce the work or apply the method to their own data with a full flexibility of tuning the number of features for their model, as long as the selection can achieve high prediction accuracy and prevent data over-interpretation. As described in the examples herein, users can check the error evolution with the number of top features to determine a reasonable range of numbers of features.

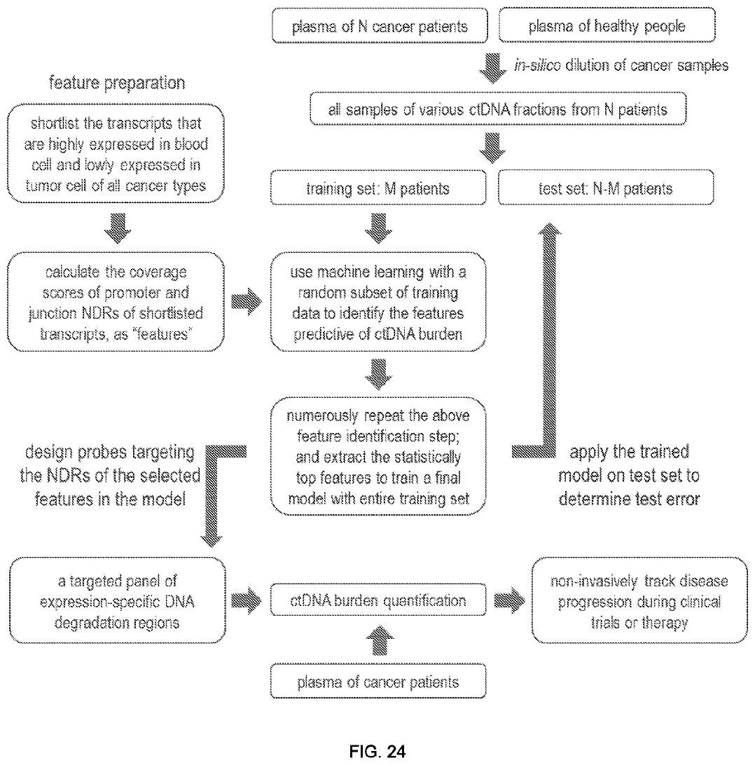

Embodiments of a machine learning model based on expression-specific DNA degradation patterns to predict ctDNA fractions for potentially clinical use are described herein (FIG. 24). Embodiments of the method enable detection of tumor DNA burden (even of very low frequency) in the blood by only sequencing these selected nucleosome-depleted regions in cfDNA assays. These regions comprise <50 kb (4 kb×6 features or 4 kb×10 features) DNA sequence in total, and may therefore allow for an extremely cost-effective approach to ctDNA content estimation (order of magnitude less DNA sequencing needed compared to standard targeted sequencing assays, usually >1000 kb). Furthermore, embodiments of the assay can be implemented as an extension/add-on to a standard targeted panel assay, allowing for an extremely cost-effective approach to generic ctDNA profiling. The colorectal and pan-cancer models have some key differences. Both colorectal cancer and pan-cancer models have high prediction accuracy, but the pan-cancer model can generalize to most/all solid tumor types (pending validation data in other cancer types).

### Results

### Overview of Approach

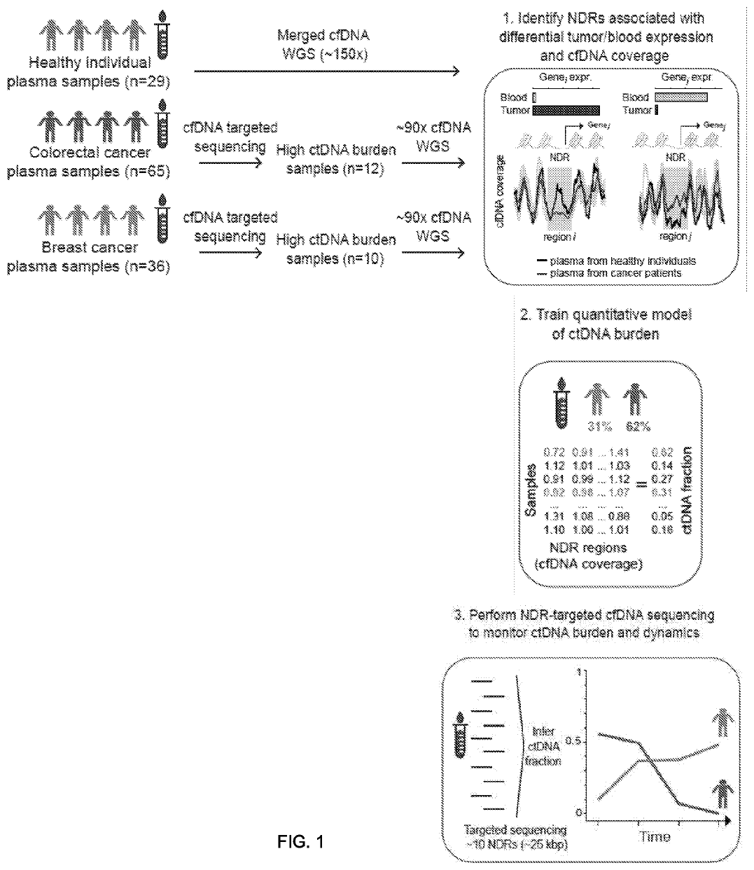

Blood samples (n=29) were collected from healthy individuals and plasma cfDNA was extracted for paired-end WGS (merged ˜150× coverage) (FIG. 1). Targeted sequencing (see Methods) of plasma samples (CRC n=65, BRCA n=36) from cancer patients was performed and samples (CRC n=12, BRCA n=10) with high SNV VAFs (indicating high ctDNA burden) were selected for deep ˜90× cfDNA WGS (Table S1). In these high ctDNA burden WGS samples, ctDNA burden estimates could be obtained using existing methods (see Methods, Oesper L, Satas G, Raphael BJ. Quantifying tumor heterogeneity in whole-genome and whole-exome sequencing data. Bioinformatics 30, 3532-3540 (2014), Ha G, et al. TITAN: inference of copy number architectures in clonal cell populations from tumor whole-genome sequence data. Genome research 24, 1881-1893 (2014), Bao L, Pu M, Messer K. AbsCN-seq: a statistical method to estimate tumor purity, ploidy and absolute copy numbers from next-generation sequencing data. Bioinformatics 30, 1056-1063 (2014) and Larson N B, Fridley B L. PurBayes: estimating tumor cellularity and subclonality in next-generation sequencing data. Bioinformatics 29, 1888-1889 (2013)) that infer tumor purity using matched tumor and germline high-depth WGS data. To identify candidate NDR features for the quantitative model, tumor and blood-specific genes with differential NDR cfDNA degradation in their promoters and first exon-intron junctions in plasma samples were identified from healthy individuals and cancer patients. Using a machine learning and in silico cfDNA generation approach, a sparse linear model was trained and tested to predict ctDNA burden from NDR cfDNA coverage. To further explore how the approach could be useful for cost-effective monitoring ctDNA dynamics, a compact (<25 kb) capture-based sequencing assay targeting predictive NDRs was designed to explore the robustness of NDR-based targeted approach using independent plasma samples (n=53) from CRC patients, and applied to estimate ctDNA levels in longitudinally collected plasma samples from a cohort of five colorectal cancer patients.

Association of Gene Expression and cfDNA Fragmentation Patterns

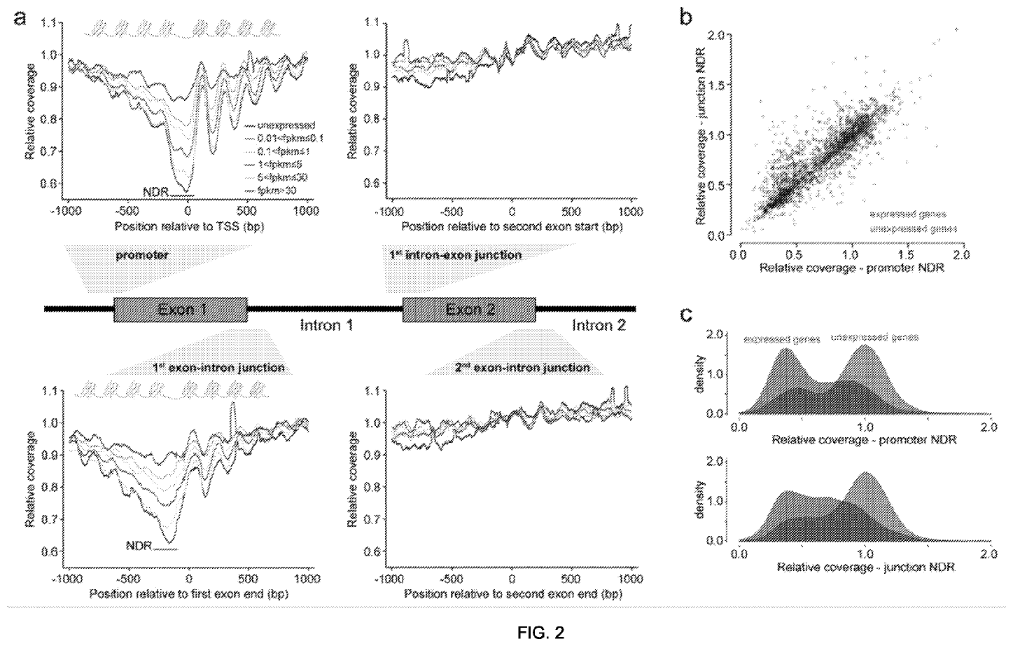

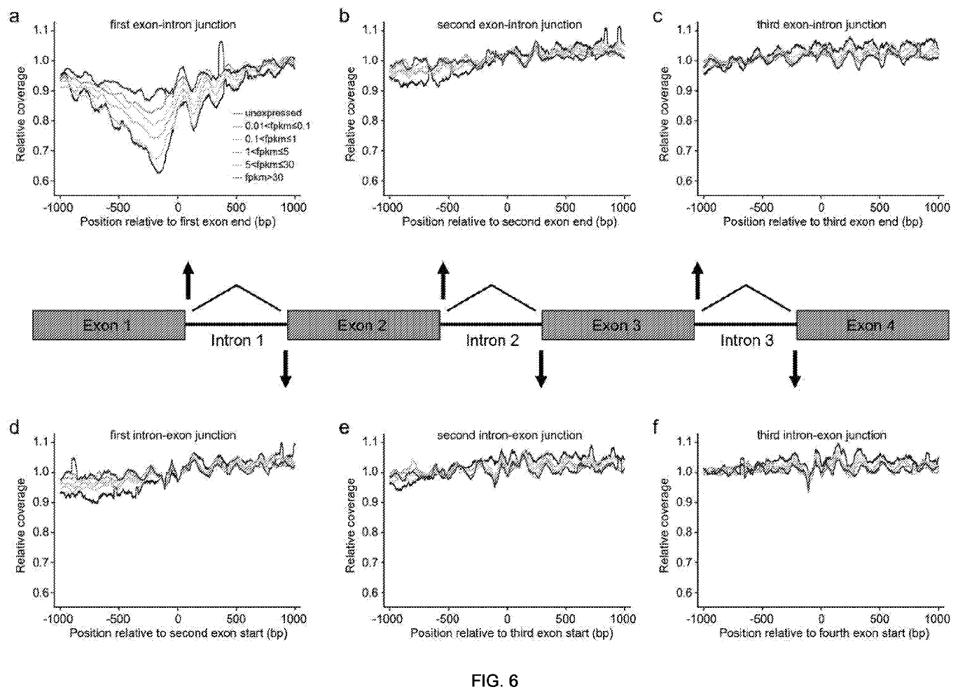

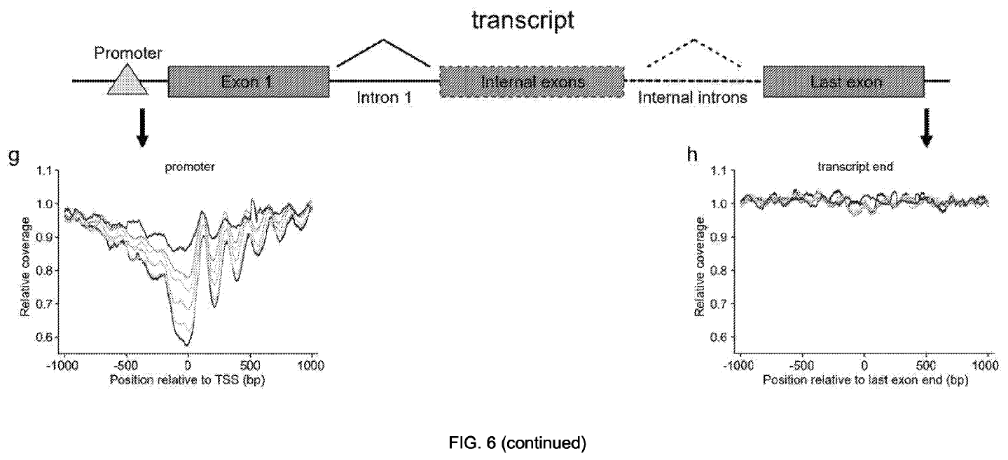

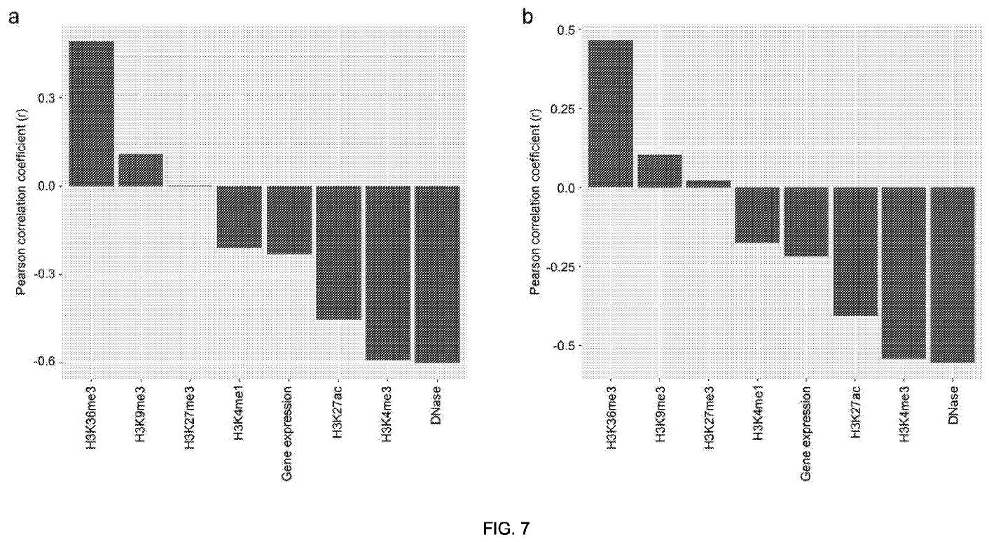

Analysis of cfDNA from the healthy individuals revealed nucleosome depletion and reduced cfDNA protection flanked by a series of strongly positioned nucleosomes at gene promoter regions (FIG. 2a). Relative coverage at the promoter NDR was inversely correlated with gene expression in whole blood cells. Studies of nucleosome positioning in cells have found that, apart from promoters, exon-intron junctions are associated with NDRs. The inventors therefore systematically scanned these gene regions for association between gene expression and cfDNA relative coverage (FIG. 2a). Strikingly, it was found that the first exon-intron junction of transcripts showed a similar association between coverage and expression, where relative cfDNA coverage at the NDR ranging from −300 to −100 bp with respect to the end of the first exon exhibited a strong inverse correlation with transcript expression in whole blood cells. However, surprisingly, correlation between expression and cfDNA coverage was not observed at other exon-intron and intron-exon junctions as well as at gene ends (FIG. 2a and FIG. 6). As expected, when comparing highly-expressed (fpkm≥30) and unexpressed gene groups, a strong positive correlation (Pearson r=0.81; Spearman correlation, p=0.85) was observed between the cfDNA relative coverage at promoter and first exon-intron junction NDRs across genes (FIG. 2b). While relative coverage at these NDRs correlated strongly with gene expression level, relative coverage could not perfectly separate unexpressed from expressed genes (FIG. 2b, c), suggesting that additional factors beyond gene expression contribute to NDR cfDNA degradation. To further explore the factors affecting cfDNA degradation at NDRs, the association between NDR relative coverage and a range of epigenetic features was explored (FIG. 7). In addition to gene expression levels (linear regression, promoter r=−0.23, junction r=−0.22), relative coverage was negatively correlated with DNase hypersensitivity (promoter r=−0.60, junction r=−0.55), H3K4me3 (promoter r=−0.59, junction r=−0.54), and H3K27ac (promoter r=−0.45, junction r=−0.41), which are markers of open chromatin, active promoters, and active enhancers respectively. In contrast, H3K36me3 (promoter r=0.49, junction r=0.46) and H3K9me3 (promoter r=0.11, junction r=0.10), markers of gene bodies and heterochromatin, were positively correlated with NDR relative coverage.

### Cf DNA Coverage Patterns at NDRs in Colorectal Cancer Patients

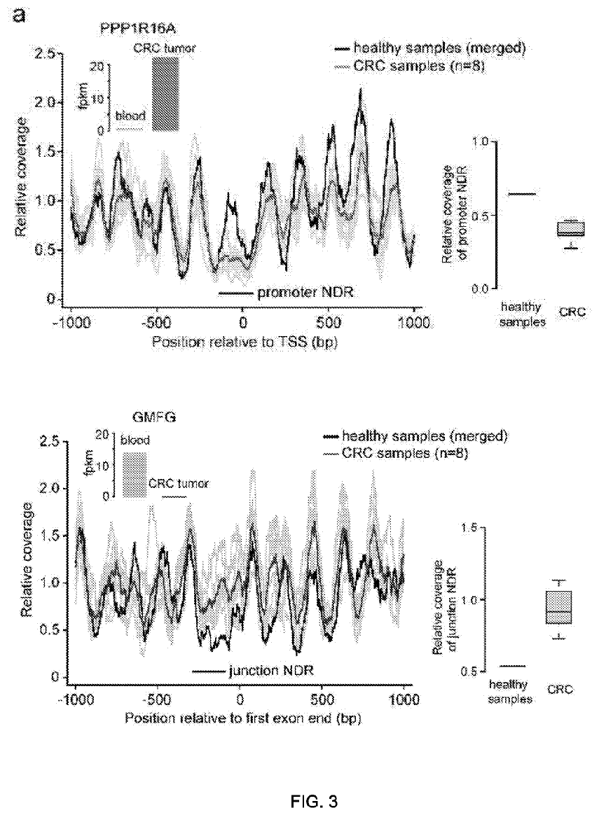

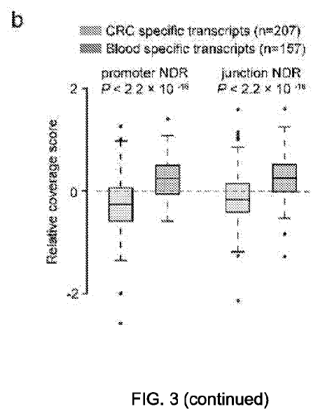

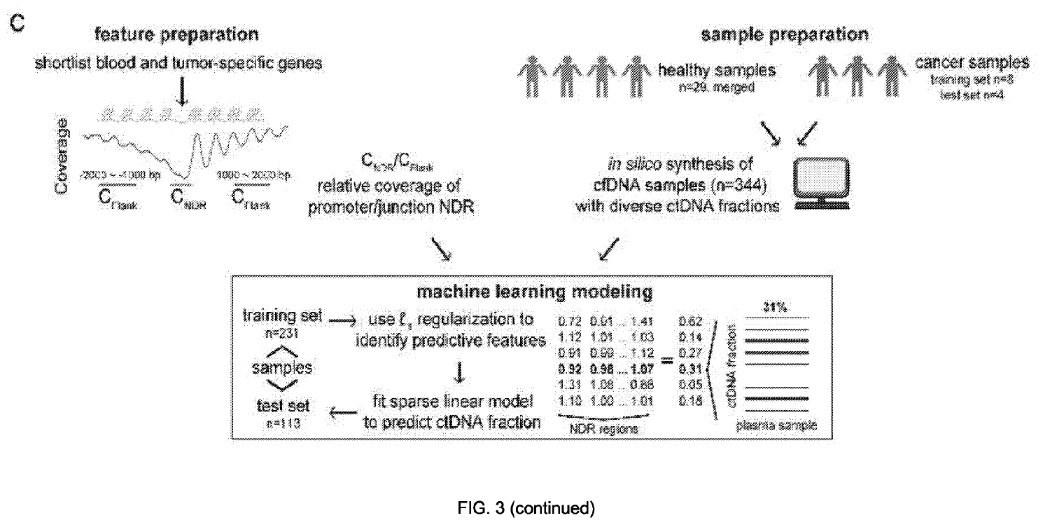

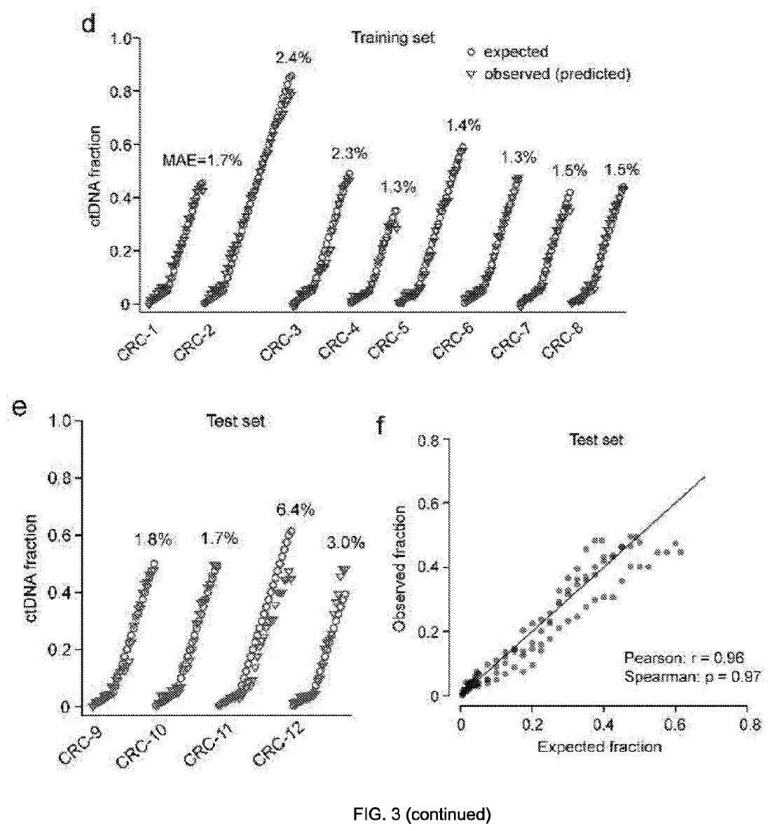

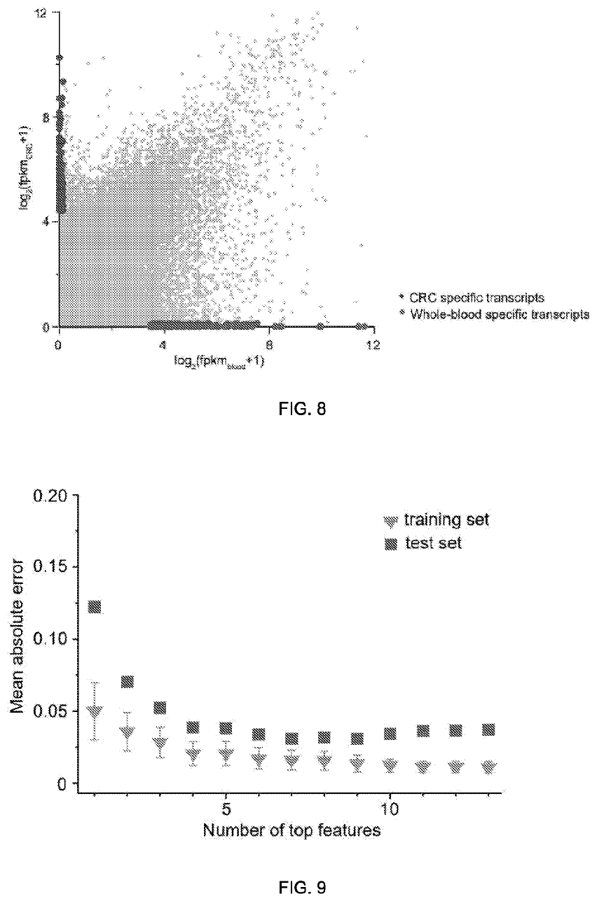

To further explore the hypothesis that NDR cfDNA coverage in plasma samples from cancer patients is associated with the epigenetic state of tumor cells, a targeted sequencing panel was first used to screen plasma samples from CRC patients for cases of high ctDNA burden (VAF >15% for known cancer driver mutations, FIG. 1). 8 plasma samples from 5 patients were initially identified and high-depth WGS was performed on these samples (˜72×-101×, Sample ID: CRC-1 to 8 in Table S1). ctDNA fractions in these samples were inferred using four existing tissue-based estimation methods (see Methods) and the median tumor purity estimate from these methods was used as ctDNA fractions (in the range 35-86%, Table S1). Gene expression data from TCGA and GTEx was then used to identify genes specifically expressed in CRC tumors and whole blood (see methods, FIG. 8). As an example, PPP1R16A was identified as a CRC-specific gene with robust depletion of NDR cfDNA coverage in plasma samples from cancer patients as compared to healthy individuals, and GMFG was identified as a blood-specific gene with greater coverage depletion in healthy blood plasma (FIG. 3a). As expected, CRC-specific genes generally showed depletion of cfDNA at both promoter and junction NDRs in the plasma of CRC patients compared to healthy controls (FIG. 3b). In contrast, blood-specific genes showed higher cfDNA coverage at NDRs in the plasma of CRC patients compared to healthy controls. Furthermore, directly comparing CRC and blood-specific genes, CRC-specific genes had significantly greater cfDNA depletion at NDRs in plasma samples from CRC patients (P<2.2×10⁻¹⁶, Wilcoxon rank-sum test, FIG. 3b).

Quantitative Estimation of Colorectal Cancer ctDNA Burden

With the insight that cfDNA coverage at NDRs is associated with the transcriptional state of DNA in the tumor cells, it was hypothesized that cfDNA coverage at a small set of NDRs could be used to infer the ctDNA burden (fraction of tumor DNA out of all cfDNA) in the blood plasma of a cancer patient. As training data, 8 deep WGS samples from 5 CRC patients were in silico “diluted” with data from healthy individuals, resulting in a training set of 231 samples of ctDNA proportions ranging from 0.5% up to the original undiluted fractions (FIG. 3c, Table S2). Candidate CRC-specific transcripts that were upregulated in CRC tumors (fpkm_{CRC}>10, fpkm_{blood}<1) and had a differential DNA degradation signal at both promoter and junction NDRs (relative coverage score <−0.2) were shortlisted. Candidate blood-specific transcripts were shortlisted with similar criteria (fpkm_{CRC}<1, fpkm_{blood}>10, relative coverage score >0.2). Relative coverages at the NDRs of these candidate transcripts were used as input features (total 529 unique tumor and 379 blood features, Table S3). Lasso L₁-regularization regression was then used in combination with a stability-based feature selection approach to a select a minimal set of 6 predictive NDRs (Table 1), which could predict the ctDNA fraction in the training data with a mean absolute error (MAE) of ˜1.8% (FIG. 3d). The signs of coefficients for the 6 NDRs in the trained model corresponded to the sign of differential expression of the associated transcripts in tumor tissue relative to whole blood (Table S4). To evaluate the ability of the model to generalize to unseen data, 4 additional samples (CRC-9 to 12 in Table S1, WGS at ˜80-95×) from 2 new CRC patients were sequenced and an in silico diluted test set of 113 samples was created (Table S2). The model accurately predicted the ctDNA proportion in this independent test set (FIG. 3e, MAE-3.4%). A direct comparison shows high similarity between the observed (predicted) and expected ctDNA fractions (FIG. 3f; Pearson r=0.96; Spearman correlation, p=0.97). To further explore the performance of more complex models, the inventors estimated the predictive error as a function of model complexity (number of top predictive features) and found that models with 4-10 NDR features were generally more accurate and better at generalizing to unseen data compared with models using fewer or more features (FIG. 9). Next, the lower limit for ctDNA detection in the NDR model was explored. Using a previous approach (Adalsteinsson V A, et al. Scalable whole-exome sequencing of cell-free DNA reveals high concordance with metastatic tumors. Nature Communications 8, 1324 (2017)), the inventors evaluated the sensitivity and specificity of the model as a function of ctDNA fraction threshold. The 113 in silico test set CRC samples (FIG. 3e, CRC-9 to 12) were used as positives, and 40 random subsets (Table S5, ˜80× each) from the data of plasma samples (n=29) from healthy individuals were used as negatives. At a 2% ctDNA fraction threshold, the model correctly predicted all positive and negative samples (100% sensitivity and specificity, Table S5). In comparison, at a 1% threshold, the sensitivity was maintained at 100% but the specificity dropped to 75%. FIG. 19 provides an overview of the machine learning feature selection, model fitting, and train/test set performance for colorectal cancer.

To further evaluate the robustness of the model when tested on in silico samples generated using healthy samples not seen during model training, the healthy samples (n=29) were split into two different groups to separately generate in silico training and test data. Reassuringly, this analysis showed robust model performance in the presence of independent train/test healthy samples (FIG. 10; test set median Pearson r=0.92; median Spearman correlation, p=0.93; median MAE=5.3%).

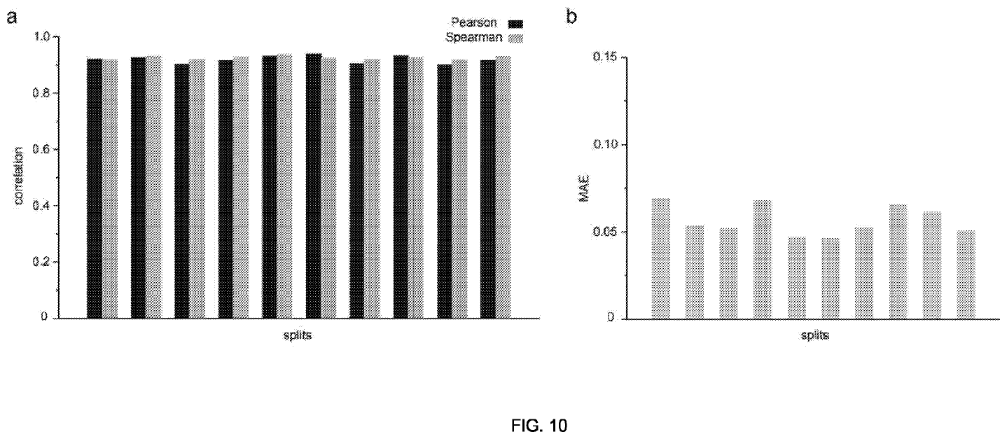

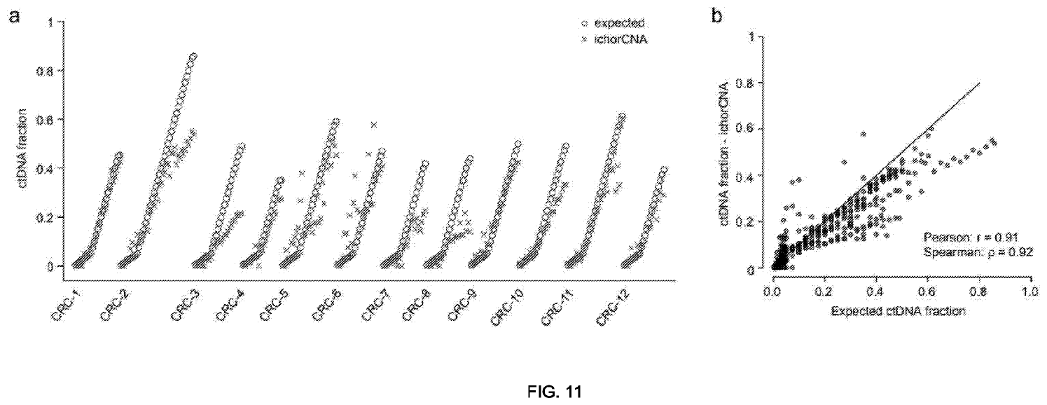

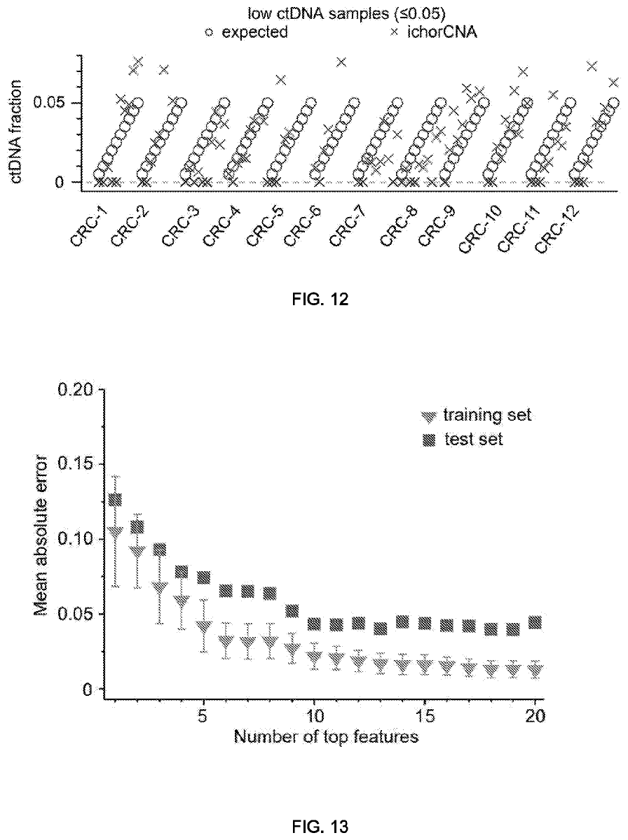

Next, the predictive performance of the model was compared with ichorCNA (Adalsteinsson V A, et al. Scalable whole-exome sequencing of cell-free DNA reveals high concordance with metastatic tumors. Nature Communications 8, 1324 (2017)), a method that estimates the ctDNA fraction on the basis of arm-level copy number alterations in low-pass WGS data. Overall, ichorCNA generally predicted comparable estimates of ctDNA burden (FIG. 11; Pearson r=0.91; Spearman correlation, p=0.92). However, while 31 out of 120 low burden samples (ctDNA burden <5%) were predicted as non-cancerous by ichorCNA (FIG. 12), only 4/120 were predicted as non-cancerous by the NDR approach. This is consistent with the reported 3% lower limit of detection using arm-level CNAs in ichorCNA (Adalsteinsson V A, et al. Scalable whole-exome sequencing of cell-free DNA reveals high concordance with metastatic tumors. Nature Communications 8, 1324 (2017)).

TABLE 1
Table 1 NDR features predictive of ctDNA fraction in CRC.

| Gene | Transcript | Chr | Site | Region | Expr. | FPKM_{blood} | FPKM_{CRC} | Pr |
|---|---|---|---|---|---|---|---|---|
| SHKBP1 | ENST00000599716 | 19 | 41082891 | junction | blood | 10.66 | 0.22 | 1.000 |
| ACSL1 | ENST00000454703 | 4 | 185747070 | junction | blood | 35.07 | 0.78 | 1.000 |
| BCAR1 | ENST00000162330 | 16 | 75285369 | junction | tumor | 0.00 | 16.86 | 1.000 |
| RAB25 | ENST00000361084 | 1 | 156030951 | promoter | tumor | 0.07 | 131.50 | 0.999 |
| PRTN3 | ENST00000234347 | 19 | 840960 | promoter | blood | 13.78 | 0.00 | 0.995 |
| LSR | ENST00000605618 | 19 | 35739922 | promoter | tumor | 0.22 | 31.85 | 0.990 |

The column Site is the position of the nucleosome-depleted site (GRCh37); Region is the annotated class of the nucleosome-depleted site (promoter or exon-intron junction); Expr. denotes whether the transcript is specifically expressed in CRC tumor tissue or whole blood cells; Pr is the probability/frequency with which the feature was selected in the Lasso stability-selection approach.

### Additional Predictive Features for Colorectal Cancer

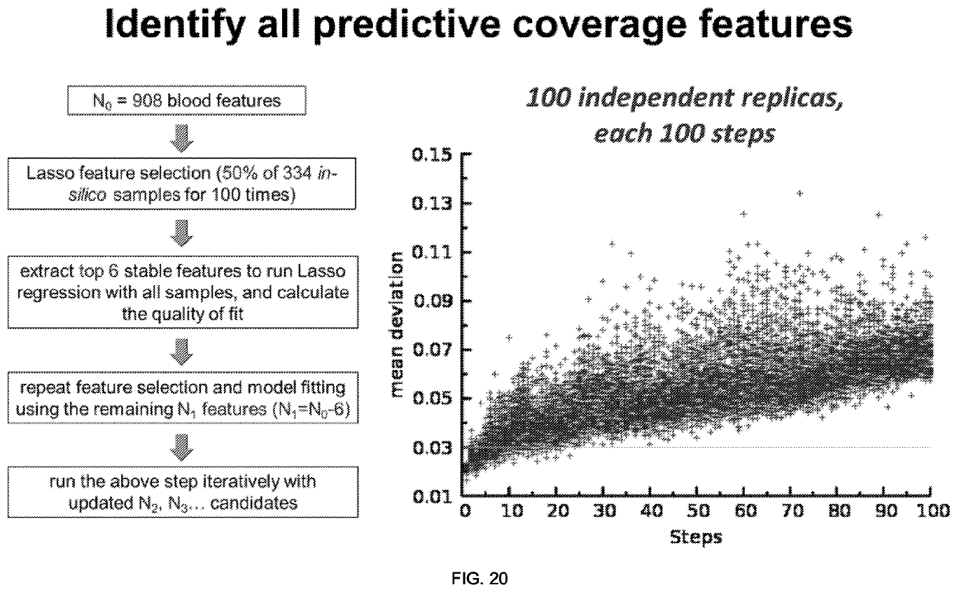

Apart from the 6 robust features identified, there may exist other predictive features that correlate with ctDNA burden. A step-wise search with Lasso regression on all 344 in silico samples was performed and the top stable 6 features in each step were extracted to estimate ctDNA fractions. The search was repeated for 100 independent times, followed by pooling all predictive features with a deviation threshold of 3% (FIG. 20). This analysis yielded 435 6-feature combinations with a predictive accuracy 3% (see Table S14), comprising a total of 158 unique features (see Table S15).

Targeted NDR Assay to Estimate ctDNA Burden

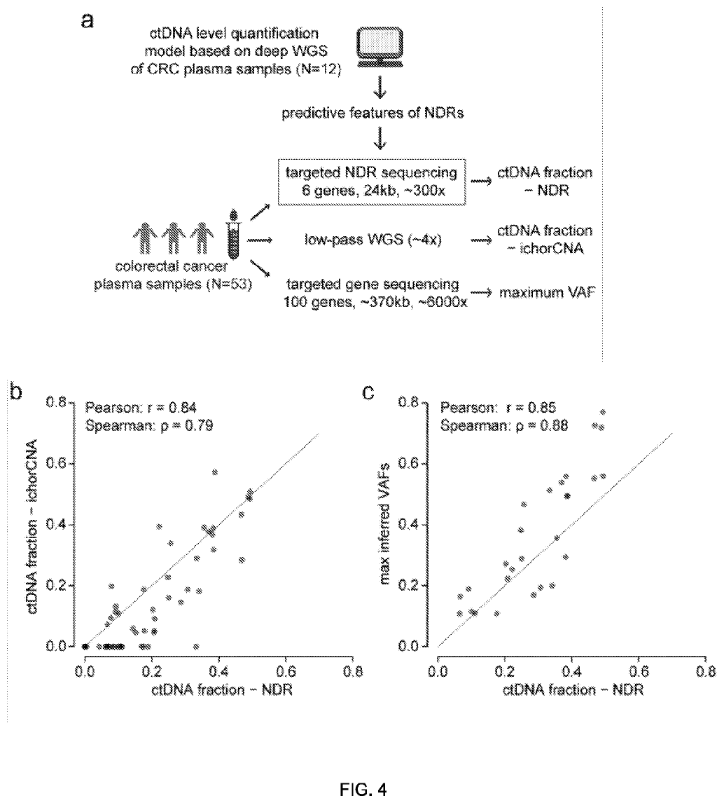

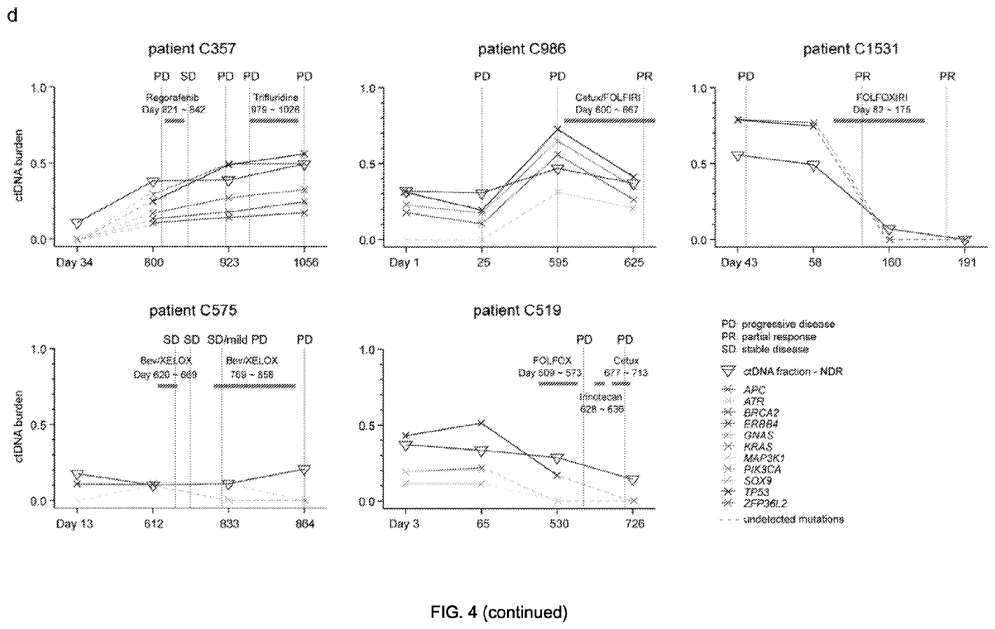

Intriguingly, since the predictive models used data from only a few NDRs, it was hypothesized that a targeted sequencing approach could be deployed for robust and low-cost estimation of ctDNA burden. The CRC model only requires cfDNA relative coverages at 6 NDRs (Table 1). The inventors therefore designed capture probes for these 6 regions (total ˜24 kb) and performed targeted sequencing (˜300×) on 53 new plasma samples from CRC patients (FIG. 4a), followed by ctDNA burden estimation from the relative coverage of the NDRs using the existing CRC model. To examine the accuracy of the model on targeted NDR sequencing data, low-pass WGS (˜4×) was also performed on the same plasma samples for ctDNA content estimation with the CNA-based method, ichorCNA. High concordance (Pearson r=0.84; Spearman correlation, p=0.79) of estimated ctDNA burden with the CNA and NDR-based approaches was observed (FIG. 4b and Table S6). Moreover, in the 53 samples (FIG. 4a, Table S6), targeted sequencing (˜6000×) of a panel of 100 frequently mutated genes (˜370 kb, Table S7) in colorectal cancer was performed. SNVs called by MuTect and VarScan were intersected and further filtered to minimize false positives (Table S8, see Methods). This analysis identified high-confidence somatic mutations in 27 plasma samples and revealed high correlation (Pearson r=0.85; Spearman correlation, p=0.88) between maximum VAFs and NDR-based ctDNA burden estimates across samples (FIG. 4c and Table S6). ctDNA was detected in 49 out of 53 (92%) samples with the targeted NDR approach, compared to 33/53 (62%) and 27/53 (51%) with ichorCNA and SNV calling approaches, respectively. The 4 ctDNA-negative samples identified with the NDR approach were also ctDNA-negative using ichorCNA and the SNV approach (Table S6). Overall, this demonstrates that the NDR-based estimation approach is robust and can be deployed with a compact and low-cost targeted sequencing approach.

NDR-Based Monitoring of ctDNA Dynamics and Disease Progression

To further explore how NDR-based ctDNA burden estimation could be used for low-cost monitoring of cancer progression, targeted NDR assay was applied to serial plasma samples collected from five CRC patients (FIG. 4d). Overall, targeted NDR profiling showed concordant ctDNA burden dynamics when compared with SNV VAFs profiled in the same samples, with coinciding increases and decreases in ctDNA burden and VAFs over time. For example, patient C357 showed generally increasing ctDNA burden and VAFs over time, and patient C986 had an intermediate coinciding peak in both ctDNA burden and VAFs. Driver mutations in TP53, PIK3CA and APC were detected in patient C986. While VAFs of these mutations were highly correlated, they showed a between-mutation spread of ˜0.1-0.2 VAF units across all timepoints. Similarly, patient C519 had TP53 and APC mutations with a ˜0.2-0.3 unit difference in VAFs. While such differences may be caused by both technical (e.g. capture efficiency) and biological (e.g. clonality or concomitant CNAs) bias, they demonstrate the challenge in estimating ctDNA burden levels based on VAFs alone.

It was noted that a number of plasma samples for which the NDR-based ctDNA burden was inferred to be positive, yet the variant calling pipeline identified no SNVs under default settings. To further understand this discordance, the raw sequencing data in these “mutation-free” plasma samples was manually inspected. Indeed, when searching for variants that were identified in other samples/timepoints from the same patients, the raw sequencing data supported presence of the expected SNVs in all the samples with positive NDR-quantified ctDNA burden (Table S9). In contrast, one plasma sample (patient C1531, day 191) was quantified with zero ctDNA burden by the NDR approach and manual screening confirmed absence of TP53 and APC mutations in this sample (Table S9). Overall, these results highlight the robustness of the targeted NDR assay for ctDNA burden estimation.

It was next explored how ctDNA burden dynamics correlate with response to targeted or cytotoxic treatments. Patient C357 was treated with Regorafenib (days 821-842 after diagnosis) followed Trifluridine (days 979-1026). However, ctDNA burden estimation in this time interval (days 800-1056) showed no drop in ctDNA burden following either treatment, indicating tumor resistance to both drugs; end-treatment CT scans (Day 916 and 1056 respectively) confirmed progressive disease. In contrast, patients with positive response to treatment showed a marked reduction of ctDNA burden in plasma. For example, patient C1531 received the chemotherapy regimen of FOLFOXIRI (days 82-175) and had on and post-treatment CT scans showing partial response. Strikingly, this patient showed a concomitant and marked drop in ctDNA burden both during (day 160) and after (day 191) treatment. In patient C575, TP53 and ATR mutations were only identified at two out of four timepoints by the pipeline. In this patient, both CT scans and ctDNA burden estimation inferred stable disease during the first round of XELOX/bevacizumab treatment (days 612-833). However, during the second round of treatment, both the ctDNA burden increased (day 864) and CT scans confirmed progressive disease, indicating acquired drug resistance. Finally, discrepancies between tumor dynamics inferred from CT imaging and ctDNA burden has previously been reported. Patient C519 reflected one such example, where CT scans indicated progressive disease while both ctDNA burden estimates and mutation VAFs decreased.

Estimation of ctDNA Burden Across Cancer Types

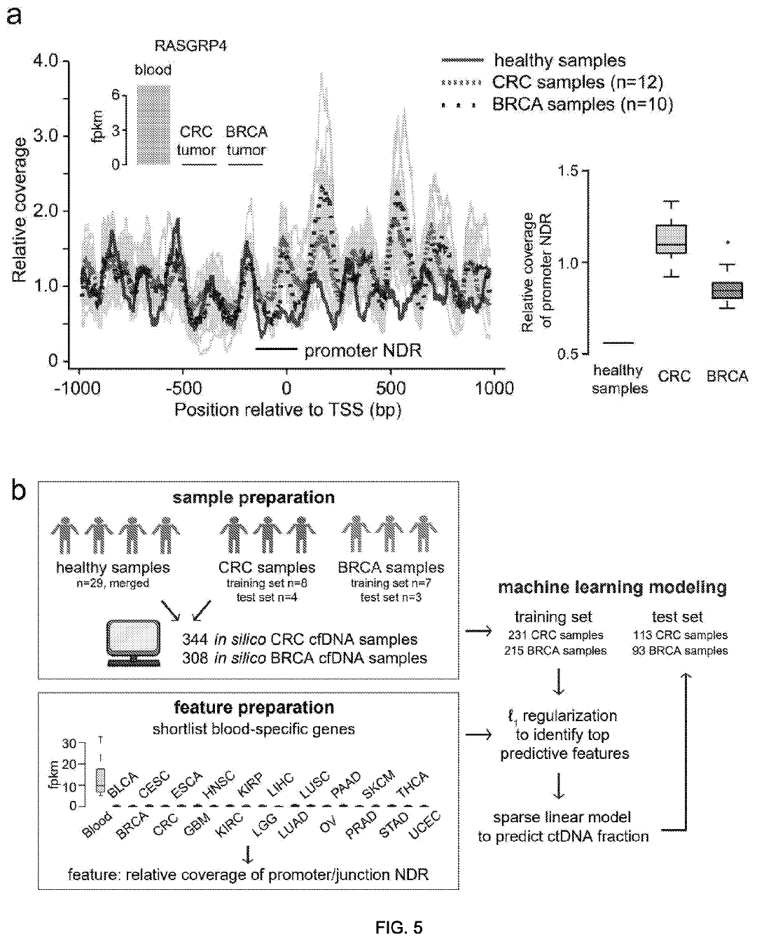

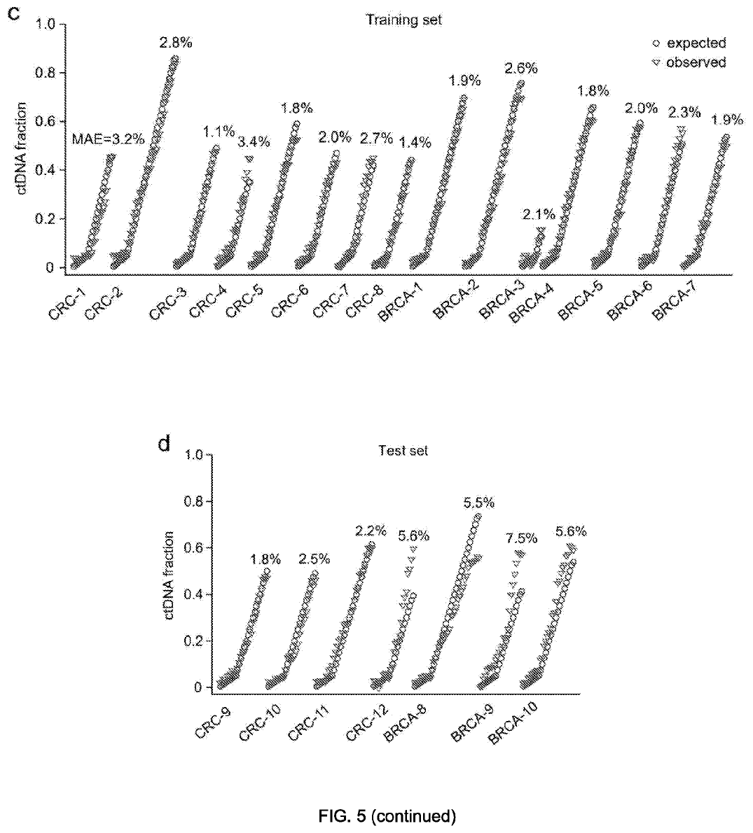

The predictive model for CRC ctDNA burden included 3 (out of 6) NDR coverage features from genes overexpressed in whole blood. Intriguingly, a predictive model completely restricted to blood-specific genes could hypothetically quantify the extent that a cfDNA profile deviates from a healthy baseline profile, allowing prediction of ctDNA burden across different cancer types. Indeed, the inventors were able to identify genes overexpressed in whole blood compared to solid tumor tissue that also had decreased NDR coverage in plasma samples from healthy individuals as compared to patients of distinct cancer types (FIG. 5a). To further systematically test this idea, deep (˜72-94×) cfDNA WGS sequencing of blood samples from 10 breast cancer patients was performed (Table S1) and # in silico diluted cfDNA samples of variable ctDNA burden were generated (Table S2, FIG. 5b). Since germline data was not available for the BRCA patients, ctDNA fractions were estimated in the 10 BRCA samples using ichorCNA. Transcripts highly overexpressed in whole blood compared to solid tumor tissues were then shortlisted (comprising 20 different solid tumor types, FIG. 5b), yielding 792 blood-specific candidate NDR features (Table S10). Using a training dataset comprising cfDNA samples from both CRC and BRCA patients, it was found that models comprising approximately 10 features were able to generalize well to unseen data from both cancer types (FIG. 13, Table 2).

TABLE 2
Table 2: Pan-cancer top-10 features. Columns/Gene: gene name/Transcript:
transcript ID/Chr: chromosome/Site: coordinate of nucleosome-depleted
site (GRCh37)/Region: location of nucleosome-depleted site/Group: gene group
based on its expression in blood and tumor/FPKM_{blood}: FPKM value in
normal blood. Their FPKM values in tumors of 20 cancer types (BLCA,
BRCA, CESC, CRC, ESCA, GBM, HNSC, KIRC, KIRP, LGG, LIHC, LUAD, LUSC,
OV, PAAD, PRAD, SKCM, STAD, THCA, and UCEC) are all <1.

| Gene | Transcript | Chr | Site | Region | Group | FPKM_{blood} |
|---|---|---|---|---|---|---|
| SLC11A1 | ENST00000465984.5 | 2 | 219246911 | promoter | blood | 10.54 |
| NLRP12 | ENST00000324134.10 | 19 | 54327597 | promoter | blood | 9.17 |
| PRTN3 | ENST00000234347.9 | 19 | 840960 | promoter | blood | 13.78 |
| HMBS | ENST00000392841.1 | 11 | 118958697 | promoter | blood | 7.28 |
| LILRB3 | ENST00000460208.1 | 19 | 54721567 | promoter | blood | 51.15 |
| ACSL1 | ENST00000513001.5 | 4 | 185678973 | junction | blood | 17.65 |
| GP9 | ENST00000307395.4 | 3 | 128779693 | junction | blood | 9.72 |
| MX2 | ENST00000398632.3 | 21 | 42774561 | promoter | blood | 7.28 |
| RASGRP4 | ENST00000615340.4 | 19 | 38916837 | promoter | blood | 6.88 |
| ATG16L2 | ENST00000542481.1 | 11 | 72534940 | promoter | blood | 25.18 |

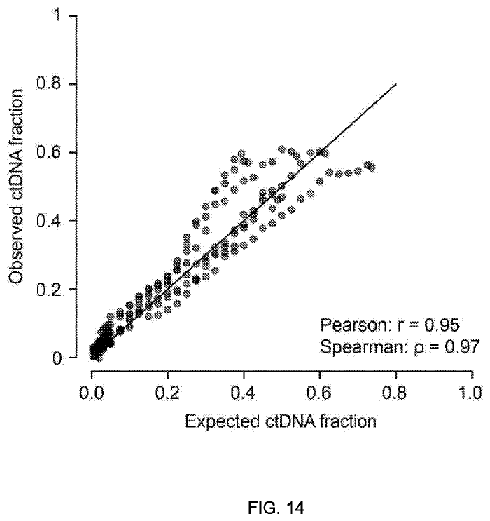

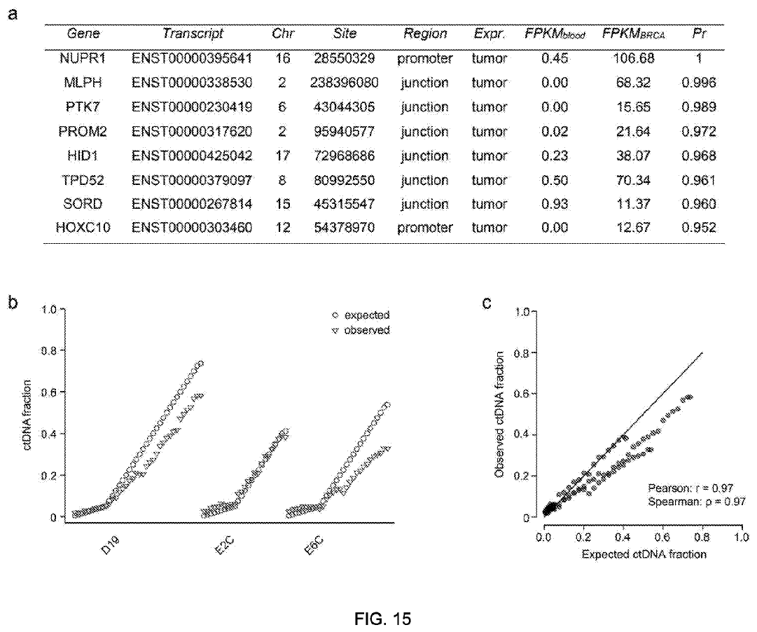

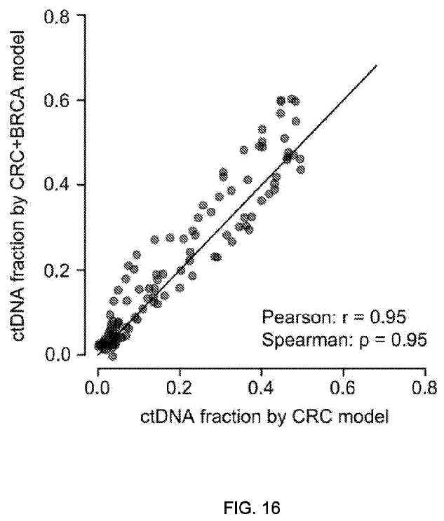

A model fitted with the training data using the top 10 predictive features (Table 2) had a mean absolute error of 2.2%, with comparable accuracy in CRC and BRCA samples (FIG. 5c). In the unseen CRC and BRCA test data (FIG. 5d), the model achieved an overall accuracy (MAE=4.3%; Pearson r=0.95; Spearman correlation, p=0.97; FIG. 14), comparable to the CRC-specific model applied to CRC data (MAE=3.4%; Pearson r=0.96; Spearman correlation, p=0.97; FIG. 3e, f) and a BRCA-specific model applied to the BRCA data (MAE=6.1%; Pearson r=0.97; Spearman correlation, p=0.97; FIG. 15). A strong concordance was also observed between the CRC+BRCA and CRC-specific models in their predicted ctDNA fractions in the test set plasma samples from CRC patients (Pearson r=0.95; Spearman correlation, p=0.95; FIG. 16). The lower limit of detection for the CRC+B RCA model was analysed by evaluating the sensitivity and specificity of the model as a function of ctDNA fraction threshold. The 206 in silico test set samples (113 CRC+93 BRCA, FIG. 5d) were used as positives and 40 random subsets (Table S11, ˜80× each) from healthy individuals were used as negatives. At a 3% ctDNA fraction detection limit, the CRC+BRCA model achieved 100% sensitivity and specificity (Table S11). In comparison, at a 2% threshold, the sensitivity was almost maintained at 99.5% but the specificity dropped to 88%. These results support that a model based on a limited set of blood-specific NDR features can predict ctDNA fractions across two distinct cancer types. FIG. 21 provides an overview of the machine learning feature selection, model fitting, and train/test set performance for pan-cancer features.

### Additional Predictive Features for Pan-Cancer

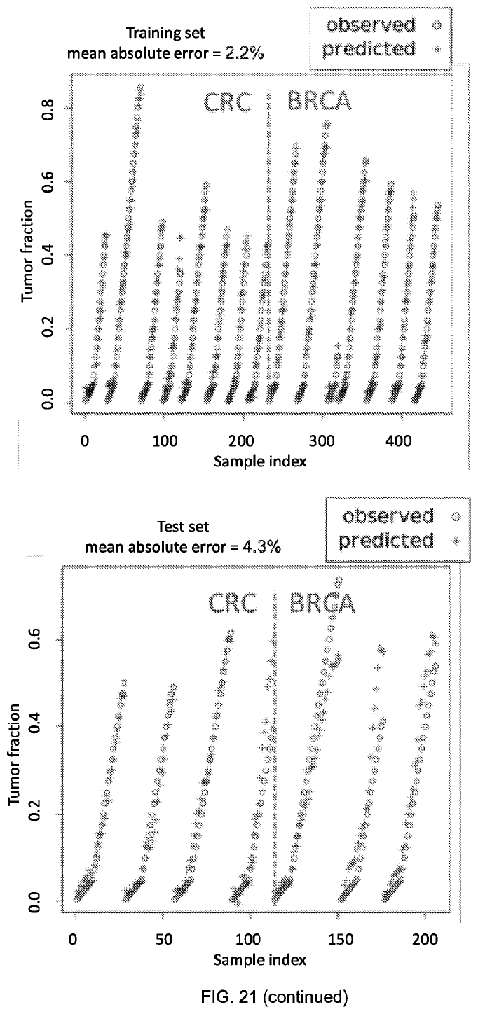

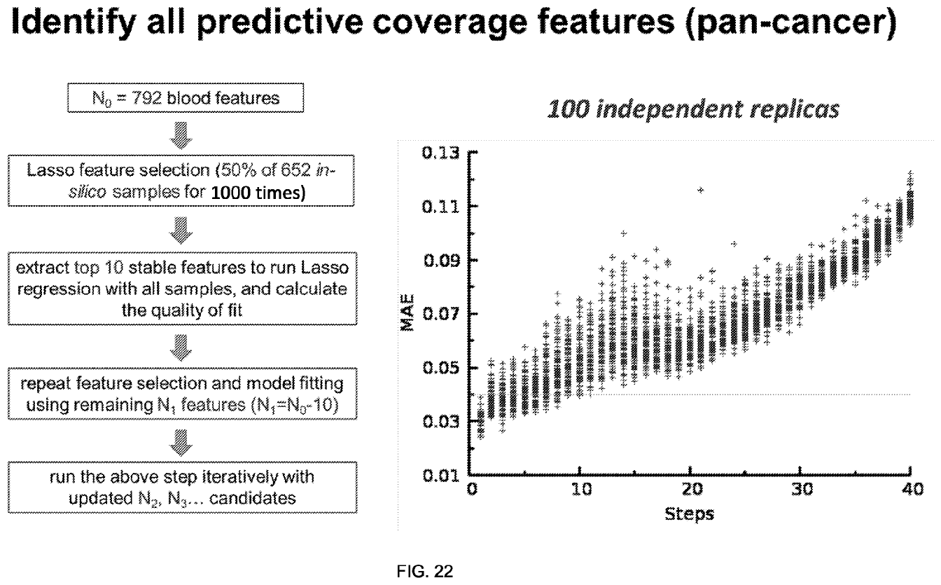

Lasso regression with all 792 blood features was employed to identify all potential predictive combination of pan-cancer features. A step-wise extensive search was carried out on all the 652 in silico samples (see Table S2), and the top 10 features in each step were extracted to estimate ctDNA content (FIG. 22). The inventors pooled all predictive features with a deviation threshold of 4% from 100 independent runs. This analysis yielded 385 10-feature combinations with a predictive accuracy ≤4% (Table S16), comprising a total of 132 unique features (Table S17).

### Additional Predictive Feature Combinations

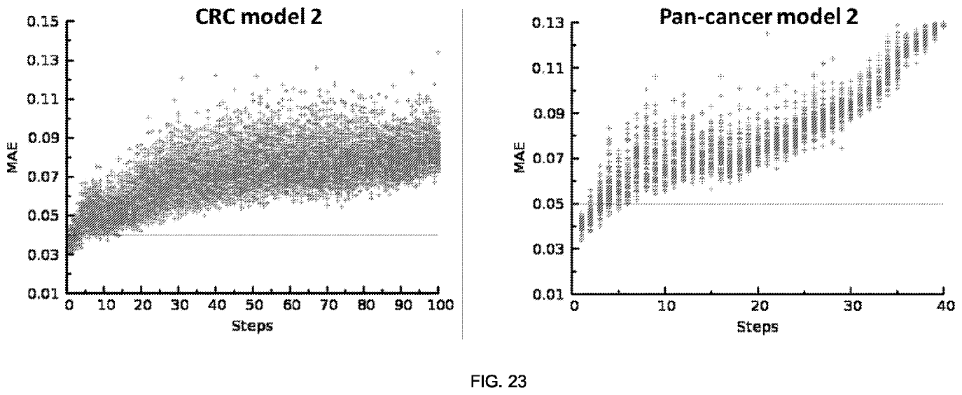

An additional search for predictive and feature combinations for both the CRC and pan-cancer models was performed. While this search was implemented as previously described (FIGS. 22 and 24), a modified approach was used to generate in silico samples, which better preserved variability in cfDNA profiles across individuals: Each cancer cfDNA sample was in silico diluted by the normal data from a random subset (n=19) of all 29 healthy samples. In this analysis, the CRC model yielded 119 new 6-feature combinations with a predictive accuracy ≤4% (FIG. 23, Table S18), comprising a total of 68 unique features (Table S19). 61 out of these 68 features were also identified as predictive in the previous CRC model (Table S15). Similarly, the pan-cancer model yielded 217 new 10-feature combinations with a predictive accuracy 5% (FIG. 23, Table S20), comprising a total of 76 unique features (Table S21) with 63/76 identified in the previous pan-cancer model (Table S17).

### Discussion

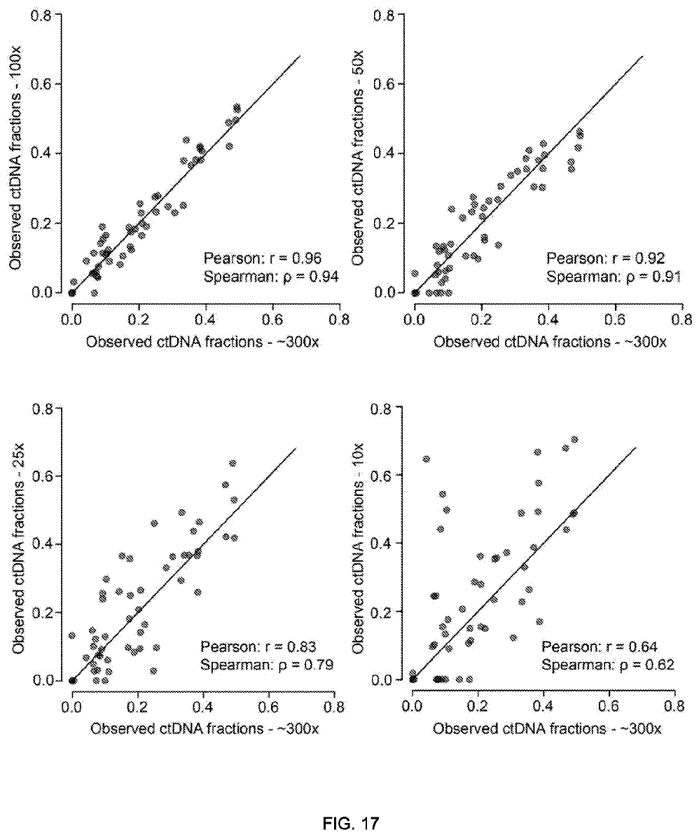

Monitoring of ctDNA offers a non-invasive approach to tracking disease progression and has been demonstrated as a valuable real-time tool for assessing therapeutic response. Here, it is shown that cfDNA coverage patterns at tumor and blood-specific NDRs can be used for quantitative estimation of the ctDNA burden in blood plasma samples. While SNV VAFs can be used as a proxy for the ctDNA burden, this only works for the subset of patients with known and measured clonal SNVs in a given targeted gene panel. SNV-based approximation of ctDNA burden may be further challenged by clonal haematopoiesis, which is frequently observed in cancer patients. Additionally, absolute ctDNA fraction estimation from SNVs requires co-estimation of allele zygosity and clonality, which may be challenging to infer for metastatic patients with multiple independently evolving tumors contributing ctDNA to the blood circulation. Furthermore, in low ctDNA burden samples, which are common and clinically important, NDR-based burden estimation showed improved accuracy as compared to a Ip-WGS-based estimation method. In contrast to Ip-WGS and DNA methylation-based profiling, NDR-based estimation is directly compatible with targeted gene panel sequencing. Since the ctDNA burden estimation model requires data from 10 or less NDRs, these regions can be profiled at low cost by capturing <25 kb of genomic sequence. Targeted cfDNA assays often cover hundreds of genes and >1 Mb captured genomic sequence, with larger panels required for profiling across cancer types and tumour mutation burden estimation. It would be straightforward to co-profile NDRs in such assays, with only a minor increment in panel size. Furthermore, down-sampling analysis showed that the NDR approach is robust down to 100× sequence coverage (FIG. 17), imposing a sequencing demand equivalent to ˜0.001×WGS, orders of magnitude lower than current Ip-WGS approaches. Importantly, an integrated NDR/gene assay would be able to estimate ctDNA burden in patients without clonal mutations in targeted cancer genes, potentially corresponding to 5-70% of patients depending on cancer type. The approach could enable low-cost and simultaneous quantitative estimation of ctDNA burden and mutational profiling in response to treatment interventions. Indeed, given the estimated lower limit of detection (˜2%) of the NDR approach, this application (i.e. simultaneous quantitative estimation of ctDNA burden and mutational profiling in response to treatment interventions) may be more relevant as compared to employing the NDR approach for screening of cancer in healthy/cancer-free individuals. Furthermore, critical for treatment decision support, independent ctDNA burden estimates could assist in classification of clonal and subclonal actionable mutations. Intriguingly, it was found that a model restricted to blood-specific NDRs could robustly predict ctDNA burden across both colorectal and breast cancer patients, suggesting it might be possible to estimate ctDNA burden independently of tumor types and metastatic lesions.

Nucleosome positioning across gene bodies, and its association with transcriptional activity, has been studied using both biochemical assays and cfDNA profiles. Unexpectedly, the systematic analysis across ordered exon-intron junctions revealed that, in addition to the promoter, only the first exon-intron junction showed signatures of strong nucleosome and expression-dependent cfDNA degradation (FIG. 2a and FIG. 6). Interestingly, transcription and splicing are coupled processes, and it has been observed that H3K4me3 and H3K9ac chromatin marks of active transcription are concentrated specifically at both promoters and ends of first exons. The data further supports that nucleosome depletion and DNA accessibility at the first exon-intron splice junction is strongly associated with transcriptional activity, supporting a model where the first exon splice region may act as a transcriptional enhancer.

In summary, the inventors have shown how tissue and expression-specific cfDNA degradation at NDRs can be used to quantitatively estimate ctDNA burden in blood samples. The approach is directly compatible with targeted gene sequencing, allowing for low-cost and simultaneous discovery of actionable cancer mutations and accurate estimation of ctDNA burden. It is anticipated that next-generation cfDNA assays based on these findings will be useful for quantitatively tracking and analysing cancer disease progression across time and patients.

### Materials and Methods

### Plasma Samples

Cancer patient and healthy volunteer samples were collected under studies 2013/110/B (now 2018/2795) and 2012/733/B approved by the Singhealth Centralised Institutional Review Board. Plasma was separated from blood within 2 hours of venipuncture via centrifugation at 10 min×300 g and 10 min×9730 g, and then stored at −80° C. DNA was extracted from plasma using the QlAamp Circulating Nucleic Acid Kit following manufacturer's instructions. Sequencing libraries were made using the KAPA HyperPrep kit (Kapa Biosystems, now Roche) following manufacturer's instructions and paired-end sequenced (2×151 bp) on either an Illumina Hiseq4000 or HiseqX.

### Whole Genome Sequencing

A targeted sequencing panel (Table S7) was first used to screen plasma samples from CRC patients and 12 samples (Table S1) of likely high ctDNA burden were selected, having maximum VAF >15% for known CRC cancer driver mutations (Supplementary Data 1). Similarly, 10 BRCA plasma samples of high ctDNA burden were selected, with either VAF >15% based on a panel of 77 genes (Table S12) of common breast cancer mutations (Supplementary Data 2), or alternatively, significant proportions (>20%) of short (length <150 bp) cfDNA fragments (Table S1). It has been reported that short cfDNA fragments below 150 bp are enriched in high-ctDNA plasma samples. Deep WGS (˜90×) was performed on the 12 cfDNA samples from 7 CRC patients and 10 cfDNA samples from 10 BRCA patients (Table S1). For the 5 CRC patients with 2 samples each, there was at least a 12 months interval between the two samples. Bwa-mem (Li H. Aligning sequence reads, clone sequences and assembly contigs with BWA-MEM. arXiv preprint arXiv:13033997, (2013)) was used to align the WGS reads from healthy (n=29, ˜5× coverage), cancer (CRC n=12, BRCA n=10, ˜90× coverage), and germline samples (CRC n=12 ˜30× coverage, not available for BRCA) were matched to the hg19 human genome. Duplicates were marked using biobambam (Tischler G, Leonard S. biobambam: tools for read pair collation based algorithms on BAM files. Source Code for Biology and Medicine 9, 13 (2014)). It has been found that trimming reads from both ends increased the coverage signal of nucleosome positioning. Similarly, the original reads (˜151 bp) were trimmed from the two ends and the central 61 bp was preserved to amplify the nucleosome-associated DNA degradation signal. BAM files of healthy individuals were merged using SAMtools merge function (Li H, et al. The sequence alignment/map format and SAMtools. Bioinformatics 25, 2078-2079 (2009)). Low-pass WGS (˜4×) was performed on 53 cfDNA samples from 23 CRC patients (Table S6).

### Sample Preparation for Targeted Sequencing

Plasma and patient-matched buffy coat samples were isolated from whole blood within six hours from collection and stored at −80° C. DNA was extracted with the QlAamp Circulating Nucleic Acid Kit, followed by library preparation using the KAPA HyperPrep kit. All libraries were tagged with custom dual indexes containing a random 8-mer unique molecular identifier. Targeted capture was performed on xGen custom panels (Integrated DNA Technologies) relevant to the experiment: a) panel of 100 genes selected based on literature review for relevance to colorectal and breast cancer, see Table S7, or b) capture probes (Supplementary Data 3) targeting genomic regions (4 kb centered at the sites in Table 1) related to the 6 NDRs predictive of ctDNA content in colorectal cancer. Paired-end sequencing (2×151 bp) was done on an Illumina Hiseq4000 machine.

### Variant Calling and Allele Frequency Estimation

Sequencing data was analyzed using the bcbio-nextgen pipeline (Guimera R V. bcbio-nextgen: Automated, distributed next-gen sequencing pipeline. EMBnetjournal 17, 30 (2012)), including read alignment with BWA mem, PCR duplicate marking with biobambam, as well as recalibration and realignment with GATK (DePristo M A, et al. A framework for variation discovery and genotyping using next-generation DNA sequencing data. Nature genetics 43, 491 (2011)). Somatic variant calling was performed using MuTect (Cibulskis K, et al. Sensitive detection of somatic point mutations in impure and heterogeneous cancer samples. Nature biotechnology 31, 213 (2013)) and VarScan (Koboldt D C, et al. VarScan: variant detection in massively parallel sequencing of individual and pooled samples. Bioinformatics 25, 2283-2285 (2009)) with default parameters, and all calls were annotated with Variant Effect Predictor (McLaren W, et al. The ensembl variant effect predictor. Genome biology 17, 122 (2016)). Variants were removed if they were outside coding regions. The inferred VAFs were either from one of the two callers if the variant was missed by one caller, or the mean if the variant was called by both callers (Table S8). Variants from HLA-A, KMT2C and MUC17 were filtered because the majority of variants in these genes were also found by at least one caller at >=0.005 VAF in buffy coat sequencing.

### Gene Expression Analysis

Tissue-specific RNA-seq transcript expression data was obtained from GTEx (including 337 whole blood samples; Table S14). Tumor RNA-seq transcript expression was obtained from TOGA (Table S14). Because a gene usually comprises multiple alternative transcripts with different genomic positions, gene expression was studied at the transcript level for a precise mapping of promoter and junction locations. Transcripts of all coding genes were grouped on the basis of their expression level (fpkm) in whole blood. If a group (e.g. 0.1<fpkm≤1; 25155 transcripts) had more than 5000 transcripts, 5000 transcripts were randomly to represent the group. Unexpressed genes were defined as transcripts that were not expressed in 99% of all 7861 GTEx samples.

### Relative Cf DNA Coverage Estimation

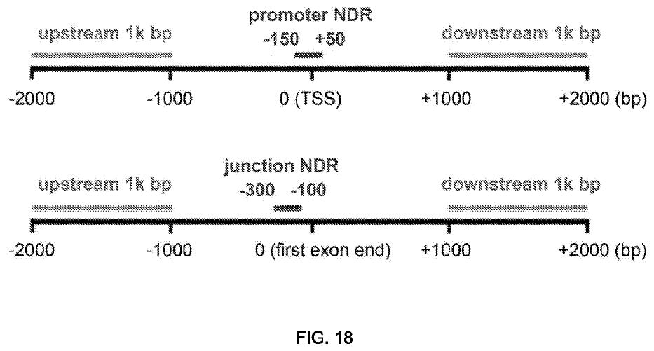

Read coverage at promoter and junction regions was computed from BAM files with SAMtools depth function (Li H, et al. The sequence alignment/map format and SAMtools. Bioinformatics 25, 2078-2079 (2009)). For the promoter region (−150 to 50 bp relative to TSS), the mean raw coverage across the region was divided (yielding “relative coverage”) by the mean coverage of the upstream (−2000˜-1000 bp relative to TSS) and downstream (1000˜2000 bp relative to TSS) flanks (FIG. 18). Thus, the mean coverage of the combined upstream and downstream 2 k bp flanks serves as a “normalization factor”. A similar approach was used for exon-intron junctions (FIG. 18). To measure the difference of relative coverage at NDRs between plasma samples from CRC patients and healthy individuals, the relative coverage score was computed:

$$score = \frac{mean ( CRC ) - m e a n ( h e a l t h y )}{\sqrt{s . d . ( CRC )}}$$

where mean(CRC) and mean(healthy) are the mean of average relative coverages at NDRs across CRC plasma and healthy plasma samples respectively, and s.d. (CRC) is the standard deviation of average relative coverages at NDRs across CRC samples. The variance in healthy samples could not be estimated due to low sequencing depth (˜5×). When computing average relative coverage of each NDR (either −150 to 50 bp relative to TSS, or −300 to −100 bp relative to first exon end), positions with relative coverage >2 were truncated to reduce bias from potential outlier values.

To explore the association between relative coverage and a range of epigenetic features, linear regression was used to fit each candidate feature (covariate) with relative coverage (response). Whole blood gene expression (fpkm) was discretized into 6 bins [unexpressed, 0.01<fpkm≤0.1, 0.1<fpkm≤1, 1<fpkm≤5, 5<fpkm≤30, fpkm >30] and fitted as a categorical covariate with the unexpressed group as the reference group. Peaks of epigenetic features [DNase, H3K4me3, H3K36me3, H3K27ac, H3K4me1, H3K9me3 and H3K27me3] from primary T-cells (E034) were obtained from the Roadmap Epigenomics Project. Epigenetic features were fitted as binary covariates with no signal as the reference group.

Estimation of ctDNA Fractions from Deep WGS cfDNA Data

The ctDNA fractions in CRC plasma samples were quantified using four different methods: THetA2, TitanCNA, AbsCN-seq and PurBayes (Oesper L, Satas G, Raphael B J. Quantifying tumor heterogeneity in whole-genome and whole-exome sequencing data. Bioinformatics 30, 3532-3540 (2014); Ha G, et al. TITAN: inference of copy number architectures in clonal cell populations from tumor whole-genome sequence data. Genome research 24, 1881-1893 (2014); Bao L, Pu M, Messer K. AbsCN-seq: a statistical method to estimate tumor purity, ploidy and absolute copy numbers from next-generation sequencing data. Bioinformatics 30, 1056-1063 (2014); Larson N B, Fridley B L. PurBayes: estimating tumor cellularity and subclonality in next-generation sequencing data. Bioinformatics 29, 1888-1889 (2013)). These methods were originally developed to use matched tumor tissue and germline Exome/WGS data to estimate mutation and copy number tumor heterogeneity, including tumor purity. Here, these methods were applied to the ˜90× cfDNA and ˜30× matched germline (buffy coat) WGS data to estimate ctDNA fractions. Somatic mutations and copy number alterations, as input to AbsCN-seq and PurBayes, were called by SMuRF (Huang W, Guo Y A, Muthukumar K, Baruah P, Chang M M, Skanderup A J. SMuRF: Portable and accurate ensemble prediction of somatic mutations. Bioinformatics (Oxford, England), (2019)) and CNVkit (Talevich E, Shain A H, Botton T, Bastian B C. CNVkit: genome-wide copy number detection and visualization from targeted DNA sequencing. PLoS Comput Biol 12, e1004873 (2016)), respectively, using the bcbio-nextgen workflow (Guimera R V. bcbio-nextgen: Automated, distributed next-gen sequencing pipeline. EMBnetjoumal 17, 30 (2012)). The median of these four ctDNA fraction estimates for a given sample was used as the final consensus estimate of the ctDNA fraction. Since germline samples were not available for the BRCA patients, the ctDNA fractions of the BRCA plasma samples were estimated by ichorCNA (Adalsteinsson V A, et al. Scalable whole-exome sequencing of cell-free DNA reveals high concordance with metastatic tumors. Nature Communications 8, 1324 (2017)).

### In Silico Sample Generation

The cancer cfDNA samples were in silico diluted by mixing cancer cfDNA reads with reads from healthy samples, maintaining the same average coverage as the original undiluted cancer cfDNA sample. The in silico generated samples were diluted from ctDNA content ranging from 0.005 up to the original undiluted fractions, with a denser sampling of low fractions 0.05 (Table S2). The inventors generated a training set of 231 samples originating from 8 samples from 5 CRC patients, and a test set of 113 samples originating from 4 samples from 2 additional CRC patients. For BRCA, the training set comprised 215 in silico generated samples from 7 patients/samples, and the test set had 93 samples from 3 patients/samples (Table S2).

### Generation of NDR Features

The relative coverage score (see above) of NDRs for all transcripts was computed and the relative coverage score was combined with expression data to shortlist tumor/blood-specific transcripts associated with differential tumor/blood NDR cfDNA coverage. For each transcript, the inventors calculated its median fpkm (fpkm_{blood}) across all whole blood samples, its median fpkm (fpkm_{CRC}) across all CRC samples, as well as its respective median fpkm values for other tumor types. Tumor transcripts were defined as being highly expressed in CRC tumor, lowly expressed in normal blood cell, and more highly degraded in CRC samples at both promoter and junction NDRs (fpkm_{CRC}>10, fpkm_{blood}<1, relative coverage score <−0.2). Blood transcripts were defined with similar rules (fpkm_{CRC}<1, fpkm_{blood}>10, relative coverage score >0.2). This approach shortlisted 284 CRC and 210 blood transcripts, each transcript with two features (promoter and junction NDR coverage). After removing overlapping features (multiple transcripts sharing the same NDR), NDR coverages of the resulting 529 tumor and 379 blood features (total n=908) were used as input features for predictive modelling. For the CRC+BRCA model, only transcripts with blood-specific expression (fpkm_{blood}>5) that were also lowly expressed (fpkm <1) in tumors of all 20 tumor types were shortlisted (TCGA tumor type acronyms: BLCA, BRCA, CESC, CRC, ESCA, GBM, HNSC, KIRC, KIRP, LGG, LIHC, LUAD, LUSC, OV, PAAD, PRAD, SKCM, STAD, THCA, UCEC), leading to a total of 792 features.

Lasso Regularized Regression to Predict ctDNA Fraction

Lasso regularized linear regression using glmnet (Friedman J, Hastie T, Tibshirani R. Regularization paths for generalized linear models via coordinate descent. Journal of statistical software 33, 1 (2010)) was used to select features and predict ctDNA content in plasma cfDNA samples. To select robust features, half of the training data was first extracted randomly and Lasso with ten-fold cross-validation was used to identify features predictive of ctDNA fractions. This procedure was repeated 1000 times and the top stable features (selection frequency 0.99) were extracted as the final predictive features, which resulted in 6 predictive features (Table 1) for the CRC-specific model and 10 predictive features (Table 2) for the CRC+BRCA model, respectively. The inventors trained the final predictive model with ten-fold cross-validation on the full training set. The inventors also attempted to predict ctDNA fractions with log-transformed relative coverage, and tested the performance using a logistic regression model, both of which failed to outperform the current model in prediction accuracy (data not shown).

To evaluate the robustness of the model when was trained and tested on in silico samples generated using independent healthy samples, the normal samples were split evenly into 2 sets. The first set (N1) was used to perform in silico spike-ins/dilution of the training set, and the second set (N2) was used for in silico dilution of the test set. Briefly, the coefficients of the CRC model (comprising the 6 features in Table 1) were re-fitted using the training data (diluted with the N1 healthy samples), and the model accuracy on the withheld test samples (diluted with N2) were then evaluated. This procedure was repeated 10 times and the model accuracy on the test data generated using the independent normal samples was evaluated.

ichorCNA Benchmarking

For the in silico samples, of DNA reads from the 12 deep-WGS CRC samples were mixed with reads from healthy samples to generate in silico low-pass samples (˜0.1×) for ctDNA content estimation using ichorCNA. The usage guidelines with default parameters were followed in the 2 step workflow: 1) read count coverage calculation with HMMcopy Suite, and 2) tumor content estimation with ichorCNA R package.

### Data Availability

cfDNA sequencing data have been deposited at the European Genome-phenome Archive (EGA) under the accession code EGAS00001004657. The data is made available for academic research. Data will be released subject to a data transfer agreement.

### SUPPLEMENTARY TABLES AND DATA

Table S1. ctDNA burden estimation of plasma samples from cancer patients.
Table S2. The in silico samples of various ctDNA content.
Table S3. Information on all candidate features of nucleosome-depleted regions for colorectal cancer.
Table S4. Coefficients for the selected NDRs in the trained models.
Table S5. Observed ctDNA fractions in the LOD analysis for the CRC model.
Table S6. CRC plasma samples for Ip-WGS and targeted sequencing.
Table S7. A panel of 100 genes frequently mutated in colorectal and breast cancer.
Table S8. Variant allele frequency estimation of CRC plasma samples.
Table S9. Mutations missed by the callers for the CRC patients with serial plasma samples.
Table S10. Information on all candidate pan-cancer features of nucleosome-depleted regions.
Table S11. Observed ctDNA fractions in the LOD analysis for the CRC+BRCA model.
Table S12. A panel of 77 genes for screening breast cancer samples.
Table S13. Transcript expression data.
Table S14. Information on all predictive features for colorectal cancer
Table S15. Information on predictive features for colorectal cancer
Table S16. Information on additional predictive pan-cancer features
Table S17. Information on predictive pan-cancer features
Table S18. All predictive feature combinations for CRC using in silico samples generated with random subsets of healthy samples.
Table S19. Information on predictive features for CRC using in silico samples generated with random subsets of healthy samples.
Table S20. All predictive pan-cancer feature combinations using in silico samples generated with random subsets of healthy samples.
Table S21. Information on predictive pan-cancer features using in silico samples generated with random subsets of healthy samples.

### Supplementary Data 1

### Supplementary Data 2

### Supplementary Data 3

TABLE S1
ctDNA burden estimation of plasma samples from cancer patients.

| | | | | tumor content estimation - | | | | tumor content estimation - | |
|---|---|---|---|---|---|---|---|---|---|
| | sample | cancer | | repetition 1 | | | | repetition 2 | |
| sample name | ID | type | coverage | theta2 | titanCNA | abscnseq | PurBayes | theta2 | titanCNA |
| 1014_180816 | CRC-1 | CRC | 80.84 | 0.38 | 0.55 | 0.48 | 0.43 | 0.38 | 0.55 |
| 1279_221015 | CRC-9 | CRC | 94.67 | 0.43 | 0.65 | 0.50 | NA | 0.43 | 0.65 |
| 1279_241116 | CRC-10 | CRC | 95.22 | 0.66 | 0.49 | NA | 0.40 | 0.66 | 0.49 |
| 1531_160616 | CRC-2 | CRC | 90.65 | 0.86 | NA | NA | 0.93 | 0.86 | 0.82 |
| 1531_180119 | CRC-3 | CRC | 100.63 | 0.67 | 0.48 | 0.32 | 0.30 | 0.75 | 0.48 |
| 512_051015 | CRC-11 | CRC | 79.96 | 0.64 | 0.69 | 0.49 | 0.44 | 0.64 | 0.69 |
| 512_130114 | CRC-12 | CRC | 79.90 | 0.27 | 0.51 | 0.44 | 0.35 | 0.27 | 0.51 |
| 519_210114 | CRC-4 | CRC | 87.27 | 0.30 | 0.40 | 0.35 | 0.36 | 0.30 | 0.40 |
| 809_030915 | CRC-5 | CRC | 75.76 | 0.45 | 0.64 | 0.59 | 0.44 | 0.45 | 0.64 |
| 809_110914 | CRC-6 | CRC | 72.56 | 0.35 | 0.59 | NA | NA | 0.35 | 0.59 |
| 986_100215 | CRC-7 | CRC | 71.63 | 0.25 | 0.69 | 0.50 | 0.36 | 0.25 | 0.69 |
| 986_261016 | CRC-8 | CRC | 76.72 | 0.34 | 0.54 | 0.51 | 0.37 | 0.34 | 0.54 |

| | tumor content estimation - | | tumor content estimation - | | | | mean | median |
|---|---|---|---|---|---|---|---|---|
| | repetition 2 | | repetition 3 | | | | ctDNA | ctDNA |
| sample name | abscnseq | PurBayes | theta2 | titanCNA | abscnseq | PurBayes | content | content |
| 1014_180816 | 0.48 | 0.43 | 0.38 | 0.55 | 0.48 | 0.43 | 0.46 | 0.45 |
| 1279_221015 | 0.50 | NA | 0.43 | 0.63 | 0.50 | 0.83 | 0.56 | 0.50 |
| 1279_241116 | NA | 0.40 | 0.66 | 0.49 | NA | 0.40 | 0.52 | 0.49 |
| 1531_160616 | NA | 0.94 | 0.85 | 0.81 | NA | 0.93 | 0.87 | 0.86 |
| 1531_180119 | 0.32 | 0.83 | 0.75 | 0.50 | 0.32 | 0.79 | 0.54 | 0.49 |
| 512_051015 | 0.49 | 0.44 | 0.59 | 0.68 | 0.49 | 0.88 | 0.60 | 0.61 |
| 512_130114 | 0.44 | 0.35 | 0.27 | 0.52 | 0.56 | 0.35 | 0.40 | 0.39 |
| 519_210114 | 0.35 | 0.36 | 0.30 | 0.43 | 0.35 | 0.35 | 0.35 | 0.35 |
| 809_030915 | 0.59 | 6.44 | 0.45 | 0.63 | 0.59 | 0.94 | 0.57 | 0.59 |
| 809_110914 | NA | NA | 0.35 | 0.60 | NA | 0.47 | 0.47 | 0.47 |
| 986_100215 | 0.50 | 0.36 | 0.25 | 0.47 | 0.50 | 0.37 | 0.43 | 0.42 |
| 986_261016 | 0.51 | 0.37 | 0.34 | 0.54 | 0.51 | 0.37 | 0.44 | 0.44 |

| sample name | sample ID | cancer type | coverage | ctDNA content estimated from ichorCNA |
|---|---|---|---|---|
| D14 | BRCA-1 | BRCA | 86.98 | 0.70 |
| D23 | BRCA-2 | BRCA | 79.41 | 0.76 |
| D7 | BRCA-3 | BRCA | 94.33 | 0.13 |
| D9 | BRCA-4 | BRCA | 89.86 | 0.66 |
| E10c | BRCA-5 | BRCA | 87.92 | 0.58 |
| E7c | BRCA-6 | BRCA | 85.45 | 0.51 |
| E8c | BRCA-7 | BRCA | 78.51 | 0.54 |
| D19 | BRCA-8 | BRCA | 76.67 | 0.74 |
| E2c | BRCA-9 | BRCA | 71.80 | 0.41 |
| E62 | BRCA-10 | BRCA | 71.50 | 0.54 |

TABLE S2
The in silica samples of various ctDNA content.

| | | | | | |
|---|---|---|---|---|---|
| 4 | posient D | sample name | sample D | MNA fraction | sample size |
| training set | 1014 | 1014,180816 | CRC-1 | 0.005, 0,010, 0,015, 0,020, 0,025, 0,030, 0,035, 0,040, 0,045, 0.050, 0.075.0.100, 0.125.0,150.0.175, 0.200, | 27 |
| | | | | 0,225. 0.250, 0.275, 0.300, 0.325, 0.350, 0.375, 0.400, 0.425, 0.450, 0.455 | |
| CRC | 1531 | 1531160616 | CRC2 | 0,005. 0.010.0.018, 0.020,0.0250.020,0.035,0.040,SMS, 0.050,0,075,0,100,5.125,0.150,0.1 IS.0.200. | 43 |
| | | | | 0.225, 0.250,0.275, 0.300,0.325,0.350.0.375.0.4000.425.0.475,0.500,0.525,0,550,S.575.0.800, | |
| | | | | 0.625, 0,660,0.675,0,700,0.725,0.750,0.775,0.800,0.825, 0.850,0.858 | |
| | 1531 | 1531,180119 | CRO3 | 0.005.0,015, 0,020, 0,025, 0,030, 0,035, 0,040, 0,045, 0.090, 0.075.0.100.0.125.0.150.0.175. 0,200, | 28 |
| | | | | 0.225. 0.250 0.275, 0.300, 0,325, 0.350,0.375, 0.400, 0.425, 0.450,0.475, 0.490 | |
| | 519 | $19,210114 | CRO-4 | 0.005, 0.010, 0.015,0.020, 0.025, 0.030, 0.035, &M0, SMS, 0.050, 0.075,0,100, 0.125,0.150,0.17S. 0.200, | 23 |
| | | | | 0.225, 5.250,0.275, 6.300,0.325, 0.350. 0.351 | |
| | 809 | 309,030915 | CRC5 | 0.005, SMS, SMS, 0.020, 0.025, 0.030.0.035.0.040 SMS. 0,050. 0.075. 0.100, 0.125.. 0.150.0.175, 0.200. | 32 |
| | | | | 0.225. 0 250,0 275,0.300), 0.325, 0.350, 0.375, S.400,0.425,0.450,0.475,0.500,0.525, 0.550. 0.575. 0.580 | |
| | 809 | 809110914 | CRC6 | 0.005. SMS, 0 615,0,020, 0.025, 0,030 SMS. 0.040, SMS. 0.050, 0.075.0.180.0.125. 0.150. 0175. 0 200, | 27 |
| | | | | 0.225, 0.258,0.275,0.300, 0.325, 0 350,0.375, 0,400,0.425, 8.450,0.469 | |
| | 986 | 988,,100215 | CR07 | SMS. SMS. SMS. 0.020,0.025 0,030.0.035. 0.040, SMS, 0.050, 0,075, 8.100,8.125.0,150, 0.1 IS. 0.200, | 25 |
| | | | | 0.226, 0.250, 0.275, 0.300,0,325, 0,350, 0.375, 0400. SMS | |
| | 988 | 986,261016 | CRC-8 | 0005, SMS, SMS, 0,020,0.025, 0,030. 6,035. QMS. QMS. 0.050, 0.075, 0 100, 0.125, 8,158,0.175, 0.200, | 26 |
| | | | | 0.225. 8 250, 8275,0.300, 0.325, 0.350, 0.375, 0.408.0.42$, 0.448 | |
| traising set | BRCA-D14 | DU | BRCA- | QMS. QMS. QMS. 0.020, 0.025, 0.030, SMS. SMS, SMS, 0.080, 0.075, 0.100, 0.125, 0.150. 0.1 IS. 0.200 | 36 |
| | | | | 0.225, 0.258,0.275,0.300. 0.325, 0.350, 8.375.0.400, S 425, SASS, 0.475, 0,500, 8.525,8.558,0.575 0.600, | |
| | | | | 0625, QMS, 0.875,0.6967 | |
| BRCA | BRCA-023 | 023 | BRCA.2 | 0005, QMS, QMS, 0.020, 0.025, 0,030.0.835. QMS. QMS. 0.050, 0,075.8.183,0.125, 0.150, 0.1 75, QMS, | 39 |
| | | | | 0.225.0,250, 0.275, 8.300,0,325, 8.350,0.375, 0,400, 0.425.0.450, 0.475, 0.500. 0.525.0.550. 0,575.0,600, | |
| | | | | 0.625, 0.658,0.675,0.700. 0.725, 0.750, 0,7567 | |
| | BRCA-O7 | 07 | BRCA-3 | 0,005. 0.010. SMS. 0.020, 0.025 0.030, 0,035, SMS, QMS, 0.050, 0.075,8,180.3.125.9,1288 | 14 |
| | BRCA-09 | 09 | 8RCA4 | 0.005. SQM, SMS, 0.020, 0.025, SMS, SMS. QMS, QMS, 0.058,0.375.0.100 0.125.8.150. 0.175 Q.2M, | 35 |
| | | | | 0.225. 0.258.0.275,0.300. 0.325. 0.350, 0,375.0.400, 0.425, CAMS, 0.475,8,500,8.526,6.668.0.57S. 0.600, | |
| | | | | 0.626, 0,850,0.6577 | |
| | BRCA-E1Qc | E1c | BRCA-5 | QMS, QMS, SMS, 0,020, 0.025, 8.030,6.035.0.040. QMS. 0.050, 0.075, 0.100, 0.125, 8.150,0.175, 0.200, | 32 |
| | | | | 0.225. 6 250, 8 275.0.300, 0.325, 0.350, 0.375, 0.408,0.42$, 0.480,0.475,0.500, 0.525.0.550. 0.575 0.5831 | |
| | 8RCA-E7c | El | BRCA-8 | 0.005. 0.010, SM5.0.020, 0.025, 0.0230, SMS, QMS, QMS, 0.990, 0.075, 0.18-0,0.12S. 0.150 3.178.3.2®. | 39 |
| | | | | QMS. 0.268,0.275.0.300, 0325,0.350, 8375, 0,400, 0,425, 0,450, 0,475, 8.500,0.5076 | |
| | BRCA/ES | EB | BRCA-T | 000 QMS.SMS, 0.020,0.025, 0.030, 0.835. QMQ. SMS>, 0,050, 0.075, 0,100, 8.125,0.158, 0.175,0.200, | 36 |
| | | | | 0.225. 6250,0.275, 0,300, 0,325, 0.350,0.375, 0.408.0.425. 0.450,0.475.0.600. 0,525.8,5356 | |

Table S2 (continued)

| | | | | | |
|---|---|---|---|---|---|
| test set CRC | 1279 | 1279,221015 | ORQ-9 | 9,005,0.010, 0.015, 0.020, 0.025, 0.030, 0.036, 0.040, 0.045, 0.050. 0.075, 0.100, 0.125. 9.150, 0.176,0.200. | 28 |
| | | | | 0.225, 0.250, 0.275, 0.300, 0.325,. 0.350. 0.375, 0.400, 0.425. 0,450, 0.475.0.500 | |
| | 1279 | 1279,241118 | CRO-10 | 0,005, 0.010, 0.015, 0.020, 0.025. 0.030, 0.035, 0.040, 0.045, 0.050, 0.075, 0.100, 0.125, 0.150, 0.175, 0.200, | 28 |
| | | | | 0.225, 0.250, 0.375,0.300, 0.325,0.350. 0.375,0,400, 0.425, 0,460, 0.475,0.490 | |
| | 512 | 512,051015 | CRO-11 | 0.006, 0.010, 0.016, 0.020, 0,025, 0.030, 0.035, 0,040, 0.945, 0,050, 0.075,0.100, 0,125, 0,150. 0.1 75, 0,200, | 33 |
| | | | | 0.225, 0.250. 0.275,0.300, 0.325. 0.350, 0.378. 0.400, 0.425, 0.-150.0.475, 0.500, 0.525. 0.550, 0.575,0.600,0.814 | |
| | 512 | 512 130114 | CRC-12 | 0.005. 0,010, 0.01 5.0.020,0.025, 0030, 0.035,0.040, 0.045,0.050. 0.075, 0.100.0,125, 0.150,0,175.0.200, | 24 |
| | | | | 0.225, 0.250, 0.275,0.300, 0.325,0.350, 0.375, 0.394 | |
| tests®! | 019 | 019 | BRCA-8 | 0.005, 0.010,0.015, 0.020,0.025, 0.030, 0.035,0.040, 0.045, 0,050, 0.075, 0.100,0.125, 0,150,0.175,0.200, | 38 } |
| | | | | 0.225, 0.250,0,275, am 0.325,0.350. am 0.400,0,425, 0.450,0.475, am 0.525,0,550, 0.575, 0,800, | |
| | | | | 0.825, 0,650, 0.575, 0.700, 0.725, 0.7:303 | |
| BRCA | E2 | E2 | BRCA9 | 0.005.0.010, 0.015.9.020.0.025, 0.030.0.635,0.040, 0.045. 0.050, 0.075.0.100. 0.125, 0.150.0.175.0.200, | 25 |
| | | | | 0.225, 0,250. 0.275, 0.300, am 0.350, 0.375.0.400, 0.4122 | |
| | Ekc | EB | BRCA-10 | 0.005, 0.010, 0.01 5,0.020, 0.025, 0.030, DM 0.040,0.045, 0.050, 0.076, 0.100, 0.125,0.150. 0.175, 0.200, | 30 |
| | | | | 6.225,0.250. 0.275, am 0.325. am am am 0,425, am 0.475, am 0.525, 0.5386 | |

TABLE S3
Information on all candidate features of nucleosome-depleted regions for colorectal cancer.

| ID | Transcript | Gene | Chr | Site | Region | Group | FPKM_{blood} | FPKMₜᵤₘₒᵣ |
|---|---|---|---|---|---|---|---|---|
| 1 | ENST00000003084.10 | CFTR | 7 | 117120017 | promoter | tumor | 0.00 | 30.20 |
| 2 | ENST00000027335.7 | CDH17 | 8 | 95220815 | promoter | tumor | 0.00 | 149.83 |
| 3 | ENST00000161006.7 | PRSS22 | 16 | 2908171 | promoter | tumor | 0.00 | 10.29 |
| 4 | ENST00000162330.9 | BCAR1 | 16 | 75285507 | promoter | tumor | 0.00 | 16.86 |
| 5 | ENST00000201586.6 | SULT2B1 | 19 | 49055332 | promoter | tumor | 0.03 | 16.67 |
| 6 | ENST00000211314.4 | TMEM14A | 6 | 52535907 | promoter | tumor | 0.67 | 25.17 |
| 7 | ENST00000215582.7 | MISP | 19 | 751126 | promoter | tumor | 0.00 | 116.54 |
| 8 | ENST00000215743.7 | MMP11 | 22 | 24115006 | promoter | tumor | 0.08 | 42.41 |
| 9 | ENST00000216968.4 | PROCR | 20 | 33759876 | promoter | tumor | 0.36 | 28.48 |
| 10 | ENST00000221992.10 | CEACAM5 | 19 | 42212537 | promoter | tumor | 0.00 | 1235.90 |
| 11 | ENST00000223271.7 | RARRES2 | 7 | 150038763 | promoter | tumor | 0.41 | 46.30 |
| 12 | ENST00000226230.7 | TMEM97 | 17 | 26646121 | promoter | tumor | 0.39 | 20.84 |
| 13 | ENST00000226760.5 | WFS1 | 4 | 6271577 | promoter | tumor | 0.35 | 15.01 |
| 14 | ENST00000227868.8 | PDHX | 11 | 34938119 | promoter | tumor | 0.99 | 13.52 |
| 15 | ENST00000228916.6 | SCNN1A | 12 | 6484715 | promoter | tumor | 0.05 | 32.36 |
| 16 | ENST00000230588.8 | MEP1A | 6 | 46761127 | promoter | tumor | 0.00 | 21.95 |
| 17 | ENST00000232458.9 | ECT2 | 3 | 172468472 | promoter | tumor | 0.28 | 12.07 |
| 18 | ENST00000245451.8 | BMP4 | 14 | 54423529 | promoter | tumor | 0.00 | 16.43 |
| 19 | ENST00000245907.10 | C3 | 19 | 6720693 | promoter | tumor | 0.37 | 15.08 |
| 20 | ENST00000250405.9 | BCL2L2 | 14 | 23775971 | promoter | tumor | 1.00 | 11.63 |
| 21 | ENST00000254260.7 | RHPN2 | 19 | 33555794 | promoter | tumor | 0.03 | 23.49 |
| 22 | ENST00000255681.6 | MACROD1 | 11 | 63933533 | promoter | tumor | 0.54 | 22.18 |
| 23 | ENST00000256585.9 | REG4 | 1 | 120354283 | promoter | tumor | 0.09 | 37.82 |
| 24 | ENST00000256951.9 | EMP1 | 12 | 13349650 | promoter | tumor | 0.39 | 14.17 |
| 25 | ENST00000260227.4 | MMP7 | 11 | 102401484 | promoter | tumor | 0.00 | 29.38 |
| 26 | ENST00000261769.9 | CDH1 | 16 | 68771128 | promoter | tumor | 0.00 | 123.30 |
| 27 | ENST00000262429.8 | ATP2C2 | 16 | 84402133 | promoter | tumor | 0.34 | 16.47 |
| 28 | ENST00000262753.8 | POF1B | X | 84634748 | promoter | tumor | 0.02 | 40.41 |
| 29 | ENST00000263629.8 | MTIF2 | 2 | 55496384 | promoter | tumor | 0.85 | 13.76 |
| 30 | ENST00000263735.8 | EPCAM | 2 | 47596287 | promoter | tumor | 0.11 | 594.54 |
| 31 | ENST00000263895.8 | RND3 | 2 | 151344208 | promoter | tumor | 0.00 | 10.29 |
| 32 | ENST00000264144.4 | LAMC2 | 1 | 183155423 | promoter | tumor | 0.01 | 31.46 |
| 33 | ENST00000264748.6 | FGFRL1 | 4 | 1006239 | promoter | tumor | 0.82 | 17.03 |
| 34 | ENST00000266980.8 | SLC39A5 | 12 | 56624436 | promoter | tumor | 0.00 | 20.87 |
| 35 | ENST00000267101.7 | ERBB3 | 12 | 56473645 | promoter | tumor | 0.00 | 40.98 |
| 36 | ENST00000267814.13 | SORD | 15 | 45315302 | promoter | tumor | 0.93 | 13.70 |
| 37 | ENST00000267996.11 | TPM1 | 15 | 63334957 | promoter | tumor | 0.87 | 32.98 |
| 38 | ENST00000269571.9 | ERBB2 | 17 | 37856333 | promoter | tumor | 0.00 | 11.47 |
| 39 | ENST00000270560.3 | TM4SF5 | 17 | 4675187 | promoter | tumor | 0.00 | 18.39 |
| 40 | ENST00000271064.11 | TINAGL1 | 1 | 32042136 | promoter | tumor | 0.10 | 42.75 |
| 41 | ENST00000278559.7 | CAPN5 | 11 | 76777988 | promoter | tumor | 0.52 | 27.31 |
| 42 | ENST00000278937.6 | MPZL2 | 11 | 118134997 | promoter | tumor | 0.37 | 17.05 |
| 43 | ENST00000288937.6 | MRPL17 | 11 | 6704632 | promoter | tumor | 0.85 | 16.44 |
| 44 | ENST00000290130.3 | MIS18A | 21 | 33651380 | promoter | tumor | 0.49 | 12.76 |
| 45 | ENST00000290913.7 | CHCHD6 | 3 | 126423063 | promoter | tumor | 0.65 | 15.07 |
| 46 | ENST00000291525.11 | TFF3 | 21 | 43735463 | promoter | tumor | 0.00 | 209.02 |
| 47 | ENST00000292401.8 | AZGP1 | 7 | 99573780 | promoter | tumor | 0.14 | 45.24 |
| 48 | ENST00000292408.8 | FGFR4 | 5 | 176513887 | promoter | tumor | 0.07 | 25.86 |
| 49 | ENST00000295092.2 | FAM84A | 2 | 14772810 | promoter | tumor | 0.00 | 13.18 |
| 50 | ENST00000296129.5 | CDCP1 | 3 | 45187914 | promoter | tumor | 0.11 | 15.27 |
| 51 | ENST00000296252.8 | LIPH | 3 | 185270401 | promoter | tumor | 0.03 | 12.23 |
| 52 | ENST00000296424.8 | BDH2 | 4 | 104021040 | promoter | tumor | 0.99 | 14.16 |
| 53 | ENST00000296666.12 | PRRC1 | 5 | 126853301 | promoter | tumor | 0.95 | 11.62 |
| 54 | ENST00000297205.6 | STEAP1 | 7 | 89783689 | promoter | tumor | 0.00 | 14.91 |
| 55 | ENST00000299610.4 | MFAP4 | 17 | 19290553 | promoter | tumor | 0.39 | 15.19 |
| 56 | ENST00000299714.7 | SEC11C | 18 | 56807107 | promoter | tumor | 0.62 | 11.38 |
| 57 | ENST00000300119.7 | MYO1A | 12 | 57443901 | promoter | tumor | 0.00 | 15.11 |
| 58 | ENST00000300283.10 | CKMT1B | 15 | 43885252 | promoter | tumor | 0.00 | 35.88 |
| 59 | ENST00000300557.2 | PRR15L | 17 | 46035244 | promoter | tumor | 0.03 | 96.21 |
| 60 | ENST00000301395.7 | GGT6 | 17 | 4463876 | promoter | tumor | 0.00 | 16.81 |
| 61 | ENST00000301887.8 | BATF2 | 11 | 64764517 | promoter | tumor | 0.43 | 11.12 |
| 62 | ENST00000307078.9 | AXIN2 | 17 | 63557765 | promoter | tumor | 0.09 | 14.10 |
| 63 | ENST00000308302.3 | GOLT1A | 1 | 204183220 | promoter | tumor | 0.03 | 12.22 |
| 64 | ENST00000308831.6 | RHOD | 11 | 66824289 | promoter | tumor | 0.07 | 10.41 |
| 65 | ENST00000310398.6 | AGR3 | 7 | 16921611 | promoter | tumor | 0.00 | 33.08 |
| 66 | ENST00000310706.9 | JUP | 17 | 39942950 | promoter | tumor | 0.87 | 86.21 |
| 67 | ENST00000310836.10 | UGT8 | 4 | 115519611 | promoter | tumor | 0.04 | 14.08 |
| 68 | ENST00000311160.13 | TNS3 | 7 | 47621742 | promoter | tumor | 0.68 | 16.18 |
| 69 | ENST00000311381.7 | C6orf203 | 6 | 107349417 | promoter | tumor | 0.93 | 14.55 |
| 70 | ENST00000311620.6 | ANKS4B | 16 | 21244986 | promoter | tumor | 0.00 | 10.61 |
| 71 | ENST00000316673.8 | HNF4A | 20 | 42984340 | promoter | tumor | 0.00 | 10.78 |
| 72 | ENST00000317508.10 | PRSS8 | 16 | 31147083 | promoter | tumor | 0.11 | 143.22 |
| 73 | ENST00000318024.8 | USH1C | 11 | 17565963 | promoter | tumor | 0.00 | 46.62 |
| 74 | ENST00000318407.4 | BOK | 2 | 242498136 | promoter | tumor | 0.57 | 13.75 |
| 75 | ENST00000318443.9 | CD276 | 15 | 73976554 | promoter | tumor | 0.00 | 11.45 |
| 76 | ENST00000318683.6 | B3GNT3 | 19 | 17905919 | promoter | tumor | 0.00 | 57.75 |
| 77 | ENST00000319838.9 | GPR35 | 2 | 241544848 | promoter | tumor | 0.31 | 30.59 |
| 78 | ENST00000322536.7 | DDX10 | 11 | 108535752 | promoter | tumor | 0.75 | 10.84 |
| 79 | ENST00000324038.6 | FAM101A | 12 | 124773710 | promoter | tumor | 0.00 | 10.08 |
| 80 | ENST00000325307.11 | HMGB3 | X | 150151759 | promoter | tumor | 0.30 | 14.09 |
| 81 | ENST00000325568.9 | IL32 | 16 | 3115298 | promoter | tumor | 0.93 | 14.52 |
| 82 | ENST00000326587.11 | MAGED1 | X | 51636698 | promoter | tumor | 0.60 | 16.14 |
| 83 | ENST00000329875.12 | PYCR1 | 17 | 79894755 | promoter | tumor | 0.12 | 36.33 |
| 84 | ENST00000331243.4 | FAM84A | 2 | 14772824 | promoter | tumor | 0.01 | 33.07 |
| 85 | ENST00000331595.8 | BGN | X | 152760397 | promoter | tumor | 0.60 | 74.92 |
| 86 | ENST00000332149.9 | TMPRSS2 | 21 | 42880086 | promoter | tumor | 0.02 | 43.57 |
| 87 | ENST00000333090.4 | TSKU | 11 | 76494283 | promoter | tumor | 0.00 | 17.21 |
| 88 | ENST00000334047.11 | F3 | 1 | 95007356 | promoter | tumor | 0.08 | 13.07 |
| 89 | ENST00000334869.8 | LGMN | 14 | 93215047 | promoter | tumor | 0.94 | 45.73 |
| 90 | ENST00000337682.8 | FAM60A | 12 | 31479306 | promoter | tumor | 0.97 | 25.73 |
| 91 | ENST00000338660.5 | VWA1 | 1 | 1370903 | promoter | tumor | 0.06 | 21.82 |
| 92 | ENST00000339276.5 | SFN | 1 | 27189629 | promoter | tumor | 0.60 | 113.82 |
| 93 | ENST00000340556.10 | MORN2 | 2 | 39103103 | promoter | tumor | 0.33 | 11.41 |
| 94 | ENST00000344616.3 | S100A14 | 1 | 153588808 | promoter | tumor | 0.00 | 143.55 |
| 95 | ENST00000350051.7 | BIRC5 | 17 | 76210279 | promoter | tumor | 0.63 | 18.32 |
| 96 | ENST00000352551.9 | UBE2C | 20 | 44441313 | promoter | tumor | 0.00 | 10.99 |
| 97 | ENST00000354624.5 | HKDC1 | 10 | 70980059 | promoter | tumor | 0.14 | 20.07 |
| 98 | ENST00000354900.7 | LSR | 19 | 35739675 | promoter | tumor | 0.75 | 31.22 |
| 99 | ENST00000355097.6 | ENTPD2 | 9 | 139948497 | promoter | tumor | 0.03 | 10.14 |
| 100 | ENST00000355808.9 | PDHA1 | X | 19362068 | promoter | tumor | 0.25 | 16.21 |
| 101 | ENST00000355899.7 | PLS3 | X | 114795509 | promoter | tumor | 0.00 | 12.05 |
| 102 | ENST00000356509.7 | GMNN | 6 | 24775159 | promoter | tumor | 0.57 | 11.97 |
| 103 | ENST00000356762.7 | CRB3 | 19 | 6464294 | promoter | tumor | 0.00 | 12.29 |
| 104 | ENST00000357166.10 | ZDHHC9 | X | 128977860 | promoter | tumor | 0.29 | 20.11 |
| 105 | ENST00000357602.7 | IPO5 | 13 | 98612503 | promoter | tumor | 0.80 | 15.40 |
| 106 | ENST00000358278.7 | TPM1 | 15 | 63334884 | promoter | tumor | 0.43 | 69.53 |
| 107 | ENST00000358432.7 | EPHA2 | 1 | 16482582 | promoter | tumor | 0.03 | 23.85 |
| 108 | ENST00000358813.4 | CCL20 | 2 | 228678570 | promoter | tumor | 0.00 | 33.26 |
| 109 | ENST00000358867.10 | TMEM126B | 11 | 85339629 | promoter | tumor | 0.47 | 17.68 |
| 110 | ENST00000360325.11 | CLDN7 | 17 | 7165797 | promoter | tumor | 0.09 | 133.65 |
| 111 | ENST00000360605.8 | URI1 | 19 | 30414551 | promoter | tumor | 0.44 | 12.86 |
| 112 | ENST00000360760.9 | SPATS2L | 2 | 201171247 | promoter | tumor | 0.73 | 32.04 |
| 113 | ENST00000360779.3 | SDCBP2 | 20 | 1309883 | promoter | tumor | 0.00 | 17.84 |
| 114 | ENST00000360798.7 | LSR | 19 | 35739668 | promoter | tumor | 0.30 | 55.63 |
| 115 | ENST00000361084.9 | RAB25 | 1 | 156030951 | promoter | tumor | 0.07 | 131.50 |
| 116 | ENST00000361852.8 | EPS8L3 | 1 | 110306546 | promoter | tumor | 0.00 | 26.64 |
| 117 | ENST00000361901.6 | CALD1 | 7 | 134464429 | promoter | tumor | 0.42 | 33.93 |
| 118 | ENST00000366787.7 | THBS2 | 6 | 169654139 | promoter | tumor | 0.07 | 14.23 |
| 119 | ENST00000366999.8 | NEK2 | 1 | 211848960 | promoter | tumor | 0.16 | 11.93 |
| 120 | ENST00000367274.8 | UBE2T | 1 | 202311108 | promoter | tumor | 0.63 | 29.13 |
| 121 | ENST00000367283.7 | ELF3 | 1 | 201979715 | promoter | tumor | 0.07 | 135.46 |
| 122 | ENST00000367284.9 | ELF3 | 1 | 201979645 | promoter | tumor | 0.02 | 50.55 |
| 123 | ENST00000367313.4 | LAD1 | 1 | 201368676 | promoter | tumor | 0.05 | 100.09 |
| 124 | ENST00000367976.3 | CTGF | 6 | 132272513 | promoter | tumor | 0.32 | 44.18 |
| 125 | ENST00000368408.3 | EFNA3 | 1 | 155051348 | promoter | tumor | 0.36 | 18.08 |
| 126 | ENST00000368554.8 | PRAP1 | 10 | 135122914 | promoter | tumor | 0.00 | 32.41 |
| 127 | ENST00000369406.7 | HMGCS2 | 1 | 120311517 | promoter | tumor | 0.00 | 17.63 |
| 128 | ENST00000370481.8 | GBP3 | 1 | 89488565 | promoter | tumor | 0.66 | 12.56 |
| 129 | ENST00000370828.3 | GPC4 | X | 132549518 | promoter | tumor | 0.03 | 13.27 |
| 130 | ENST00000371221.3 | ALDH18A1 | 10 | 97416463 | promoter | tumor | 0.04 | 29.55 |
| 131 | ENST00000372486.5 | NTMT1 | 9 | 132371163 | promoter | tumor | 0.00 | 14.12 |
| 132 | ENST00000372966.7 | NOX1 | X | 100129334 | promoter | tumor | 0.00 | 48.52 |
| 133 | ENST00000373255.8 | H2AFY2 | 10 | 71812552 | promoter | tumor | 0.42 | 12.60 |
| 134 | ENST00000373290.6 | TSPAN15 | 10 | 71211229 | promoter | tumor | 0.30 | 60.96 |
| 135 | ENST00000373669.6 | PIN4 | X | 71401529 | promoter | tumor | 0.87 | 13.81 |
| 136 | ENST00000373699.5 | PPIL1 | 6 | 36842800 | promoter | tumor | 0.85 | 17.26 |
| 137 | ENST00000373944.7 | ZWINT | 10 | 58121036 | promoter | tumor | 0.38 | 16.27 |
| 138 | ENST00000374183.4 | BSPRY | 9 | 116111821 | promoter | tumor | 0.22 | 15.59 |
| 139 | ENST00000374214.3 | UQCC2 | 6 | 33679504 | promoter | tumor | 1.00 | 17.77 |
| 140 | ENST00000374837.7 | MAP1LC3A | 20 | 33134658 | promoter | tumor | 0.87 | 16.45 |
| 141 | ENST00000375431.8 | GRTP1 | 13 | 114018441 | promoter | tumor | 0.05 | 18.16 |
| 142 | ENST00000376569.7 | DDR1 | 6 | 30851846 | promoter | tumor | 0.08 | 11.00 |
| 143 | ENST00000376652.8 | ENTPD6 | 20 | 25176356 | promoter | tumor | 0.44 | 14.06 |
| 144 | ENST00000378115.2 | ARHGEF35 | 7 | 143892748 | promoter | tumor | 0.10 | 13.16 |
| 145 | ENST00000378378.8 | ARHGEF16 | 1 | 3370990 | promoter | tumor | 0.00 | 12.12 |
| 146 | ENST00000378427.5 | FAM213B | 1 | 2518237 | promoter | tumor | 0.59 | 12.06 |
| 147 | ENST00000379046.6 | NQO1 | 16 | 69760487 | promoter | tumor | 0.07 | 39.35 |
| 148 | ENST00000379047.7 | NQO1 | 16 | 69760571 | promoter | tumor | 0.09 | 34.23 |
| 149 | ENST00000379715.9 | EEF1E1 | 6 | 8102811 | promoter | tumor | 0.59 | 17.82 |
| 150 | ENST00000379742.4 | POSTN | 13 | 38172895 | promoter | tumor | 0.01 | 11.69 |
| 151 | ENST00000379805.3 | PDHA1 | X | 19362086 | promoter | tumor | 0.76 | 11.58 |
| 152 | ENST00000379923.5 | ACO1 | 9 | 32384618 | promoter | tumor | 0.81 | 16.28 |
| 153 | ENST00000380071.7 | RFC3 | 13 | 34392186 | promoter | tumor | 0.48 | 15.11 |
| 154 | ENST00000381134.7 | ARSE | X | 2882311 | promoter | tumor | 0.00 | 24.51 |
| 155 | ENST00000382848.4 | GJB2 | 13 | 20767037 | promoter | tumor | 0.09 | 18.88 |
| 156 | ENST00000389614.5 | GPX2 | 14 | 65409531 | promoter | tumor | 0.10 | 652.10 |
| 157 | ENST00000391967.6 | LAD1 | 1 | 201368736 | promoter | tumor | 0.00 | 16.64 |
| 158 | ENST00000393316.7 | BCL2L15 | 1 | 114430169 | promoter | tumor | 0.11 | 11.11 |
| 159 | ENST00000393366.6 | ATP5G1 | 17 | 46970179 | promoter | tumor | 0.63 | 40.08 |
| 160 | ENST00000393725.6 | KIAA1191 | 5 | 175788768 | promoter | tumor | 0.75 | 11.70 |
| 161 | ENST00000394201.8 | SCOC | 4 | 141294797 | promoter | tumor | 0.53 | 14.61 |
| 162 | ENST00000394265.5 | PPP1R1B | 17 | 37784751 | promoter | tumor | 0.00 | 81.30 |
| 163 | ENST00000395641.2 | NUPR1 | 16 | 28550329 | promoter | tumor | 0.45 | 37.05 |
| 164 | ENST00000397542.6 | CDHR5 | 11 | 625067 | promoter | tumor | 0.02 | 18.38 |
| 165 | ENST00000397714.6 | SEPTIN10 | 2 | 110371504 | promoter | tumor | 0.14 | 14.96 |
| 166 | ENST00000397995.2 | RNASE4 | 14 | 21152742 | promoter | tumor | 0.56 | 10.20 |
| 167 | ENST00000398599.8 | CEACAM5 | 19 | 42212504 | promoter | tumor | 0.00 | 74.52 |
| 168 | ENST00000401412.5 | AGR2 | 7 | 16844611 | promoter | tumor | 0.00 | 103.38 |
| 169 | ENST00000403444.7 | CEACAM1 | 19 | 43032630 | promoter | tumor | 0.00 | 33.67 |
| 170 | ENST00000405271.5 | EPCAM | 2 | 47572297 | promoter | tumor | 0.00 | 20.99 |
| 171 | ENST00000405816.5 | CEACAM5 | 19 | 42212568 | promoter | tumor | 0.00 | 10.92 |
| 172 | ENST00000409189.7 | CCL20 | 2 | 228678558 | promoter | tumor | 0.00 | 20.73 |
| 173 | ENST00000410036.2 | MZT2A | 2 | 132250316 | promoter | tumor | 0.00 | 12.64 |
| 174 | ENST00000416348.1 | ADIRF | 10 | 88728189 | promoter | tumor | 0.00 | 22.43 |
| 175 | ENST00000419304.6 | AGR2 | 7 | 16844704 | promoter | tumor | 0.00 | 257.72 |
| 176 | ENST00000419308.6 | FOXA2 | 20 | 22565101 | promoter | tumor | 0.00 | 13.51 |
| 177 | ENST00000420892.1 | HTRA1 | 10 | 124266207 | promoter | tumor | 0.98 | 24.46 |
| 178 | ENST00000423485.5 | TOP2A | 17 | 38574202 | promoter | tumor | 0.44 | 31.06 |
| 179 | ENST00000425042.6 | HID1 | 17 | 72968829 | promoter | tumor | 0.23 | 13.99 |
| 180 | ENST00000425340.2 | FUT2 | 19 | 49199232 | promoter | tumor | 0.02 | 13.10 |
| 181 | ENST00000427250.5 | LSR | 19 | 35739905 | promoter | tumor | 0.00 | 13.66 |
| 182 | ENST00000428445.1 | VARS | 6 | 31750366 | promoter | tumor | 0.78 | 10.22 |
| 183 | ENST00000428849.6 | KIFC1 | 6 | 33359313 | promoter | tumor | 0.60 | 17.24 |
| 184 | ENST00000429772.6 | TMEM106C | 12 | 48357403 | promoter | tumor | 0.70 | 34.02 |
| 185 | ENST00000430118.1 | HMGB3 | X | 150154004 | promoter | tumor | 0.52 | 12.82 |
| 186 | ENST00000433307.2 | PLEKHA1 | 10 | 124152644 | promoter | tumor | 0.98 | 13.38 |
| 187 | ENST00000439109.6 | NQO1 | 16 | 69760463 | promoter | tumor | 0.00 | 10.97 |
| 188 | ENST00000440815.7 | IL32 | 16 | 3115370 | promoter | tumor | 0.34 | 22.19 |
| 189 | ENST00000441275.5 | BDH1 | 3 | 197282858 | promoter | tumor | 0.87 | 17.80 |
| 190 | ENST00000444124.6 | DDC | 7 | 50633154 | promoter | tumor | 0.00 | 21.66 |
| 191 | ENST00000446188.1 | ELF3 | 1 | 201979772 | promoter | tumor | 0.00 | 13.93 |
| 192 | ENST00000448599.2 | PHGR1 | 15 | 40643234 | promoter | tumor | 0.00 | 289.14 |
| 193 | ENST00000450427.1 | PRR15 | 7 | 29605066 | promoter | tumor | 0.00 | 10.59 |
| 194 | ENST00000450894.7 | ITGB4 | 17 | 73717577 | promoter | tumor | 0.14 | 50.86 |
| 195 | ENST00000452441.5 | DDR1 | 6 | 30852327 | promoter | tumor | 0.07 | 51.62 |
| 196 | ENST00000455712.5 | POLR2H | 3 | 184080403 | promoter | tumor | 0.94 | 11.92 |
| 197 | ENST00000463201.2 | PRAP1 | 10 | 135164879 | promoter | tumor | 0.00 | 54.46 |
| 198 | ENST00000467415.5 | TMEM14C | 6 | 10723358 | promoter | tumor | 0.69 | 26.73 |
| 199 | ENST00000467905.5 | AK2 | 1 | 33502487 | promoter | tumor | 0.00 | 11.87 |
| 200 | ENST00000469862.1 | CENPF | 1 | 214828491 | promoter | tumor | 0.39 | 10.26 |
| 201 | ENST00000472782.1 | ATP5G3 | 2 | 176046423 | promoter | tumor | 0.62 | 10.87 |
| 202 | ENST00000473336.5 | RAB25 | 1 | 156031174 | promoter | tumor | 0.00 | 17.65 |
| 203 | ENST00000476587.1 | NDUFB5 | 3 | 179334763 | promoter | tumor | 0.89 | 14.92 |
| 204 | ENST00000478194.1 | FERMT1 | 20 | 6074819 | promoter | tumor | 0.00 | 27.00 |
| 205 | ENST00000478869.1 | WDR12 | 2 | 203765843 | promoter | tumor | 0.71 | 14.50 |
| 206 | ENST00000479419.1 | IFT172 | 2 | 27669072 | promoter | tumor | 0.00 | 37.10 |
| 207 | ENST00000484713.1 | LAMB2 | 3 | 49159029 | promoter | tumor | 0.41 | 13.87 |
| 208 | ENST00000489477.1 | NDUFAF4 | 6 | 97345750 | promoter | tumor | 0.66 | 10.30 |
| 209 | ENST00000489523.5 | AGR2 | 7 | 16844623 | promoter | tumor | 0.00 | 64.63 |
| 210 | ENST00000490807.5 | NTPCR | 1 | 233086360 | promoter | tumor | 0.77 | 10.54 |
| 211 | ENST00000494163.1 | IFT172 | 2 | 27669063 | promoter | tumor | 0.00 | 11.52 |
| 212 | ENST00000494446.1 | FN1 | 2 | 216230399 | promoter | tumor | 0.79 | 52.10 |
| 213 | ENST00000494801.5 | TCEAL4 | X | 102840491 | promoter | tumor | 0.60 | 13.59 |
| 214 | ENST00000495558.1 | VWA1 | 1 | 1370241 | promoter | tumor | 0.00 | 13.92 |
| 215 | ENST00000495848.1 | ELF3 | 1 | 201979722 | promoter | tumor | 0.00 | 17.88 |
| 216 | ENST00000496195.1 | GLRX3 | 10 | 131977638 | promoter | tumor | 0.98 | 10.30 |
| 217 | ENST00000497734.5 | SRC | 20 | 35973088 | promoter | tumor | 0.00 | 12.44 |
| 218 | ENST00000507614.1 | TIMM8B | 11 | 111957446 | promoter | tumor | 0.80 | 10.54 |
| 219 | ENST00000507699.1 | PALLD | 4 | 169819128 | promoter | tumor | 0.31 | 14.56 |
| 220 | ENST00000514985.5 | SEPP1 | 5 | 42812181 | promoter | tumor | 0.59 | 49.95 |
| 221 | ENST00000520271.5 | COX6C | 8 | 100905850 | promoter | tumor | 0.51 | 12.86 |
| 222 | ENST00000523677.5 | C1orf210 | 1 | 43751250 | promoter | tumor | 0.00 | 13.49 |
| 223 | ENST00000524832.5 | CHID1 | 11 | 902322 | promoter | tumor | 0.42 | 10.82 |
| 224 | ENST00000525657.1 | C1QTNF5 | 11 | 119211508 | promoter | tumor | 0.05 | 17.27 |
| 225 | ENST00000526202.5 | LMO7 | 13 | 76334795 | promoter | tumor | 0.09 | 23.11 |
| 226 | ENST00000527020.5 | USH1C | 11 | 17565947 | promoter | tumor | 0.00 | 10.90 |
| 227 | ENST00000527106.5 | FUT6 | 19 | 5838771 | promoter | tumor | 0.00 | 13.22 |
| 228 | ENST00000528430.2 | PPP1R16A | 8 | 145726472 | promoter | tumor | 0.92 | 22.33 |
| 229 | ENST00000530094.5 | CTNND1 | 11 | 57529269 | promoter | tumor | 0.00 | 26.61 |
| 230 | ENST00000532203.1 | MRPL17 | 11 | 6704547 | promoter | tumor | 0.65 | 21.73 |
| 231 | ENST00000533827.5 | VPS51 | 11 | 64876763 | promoter | tumor | 0.00 | 12.01 |
| 232 | ENST00000534378.5 | ILVBL | 19 | 15236533 | promoter | tumor | 0.64 | 11.41 |
| 233 | ENST00000537496.5 | MMAB | 12 | 110011308 | promoter | tumor | 0.93 | 12.43 |
| 234 | ENST00000541754.1 | NNMT | 11 | 114168085 | promoter | tumor | 0.72 | 23.55 |
| 235 | ENST00000542056.1 | GPRC5A | 12 | 13044513 | promoter | tumor | 0.00 | 23.91 |
| 236 | ENST00000543445.5 | LDHA | 11 | 18416107 | promoter | tumor | 0.00 | 71.47 |
| 237 | ENST00000543623.5 | PLCD3 | 17 | 43192496 | promoter | tumor | 0.27 | 14.58 |
| 238 | ENST00000544913.2 | HMGCS2 | 1 | 120311528 | promoter | tumor | 0.03 | 21.61 |
| 239 | ENST00000546314.5 | STARD10 | 11 | 72493404 | promoter | tumor | 0.00 | 31.66 |
| 240 | ENST00000546485.5 | RPL41 | 12 | 56510416 | promoter | tumor | 0.75 | 48.15 |
| 241 | ENST00000547281.5 | CDK4 | 12 | 58146059 | promoter | tumor | 0.83 | 30.31 |
| 242 | ENST00000547838.2 | FAM109A | 12 | 111801553 | promoter | tumor | 0.94 | 11.49 |
| 243 | ENST00000548169.2 | ATP2A2 | 12 | 110729854 | promoter | tumor | 0.34 | 13.63 |
| 244 | ENST00000552128.2 | TSPAN8 | 12 | 71533055 | promoter | tumor | 0.00 | 15.81 |
| 245 | ENST00000552561.5 | TMEM106C | 12 | 48357388 | promoter | tumor | 0.38 | 17.25 |
| 246 | ENST00000553237.5 | CDK4 | 12 | 58146109 | promoter | tumor | 0.06 | 16.39 |
| 247 | ENST00000554989.1 | CKB | 14 | 103987659 | promoter | tumor | 0.75 | 83.82 |
| 248 | ENST00000557049.1 | GPX2 | 14 | 65409438 | promoter | tumor | 0.00 | 18.28 |
| 249 | ENST00000558580.1 | SORD | 15 | 45328434 | promoter | tumor | 0.95 | 15.73 |
| 250 | ENST00000561504.1 | ADIRF | 10 | 88728247 | promoter | tumor | 0.75 | 46.05 |
| 251 | ENST00000562684.5 | HN1L | 16 | 1728278 | promoter | tumor | 0.25 | 19.21 |
| 252 | ENST00000581920.1 | TYMS | 18 | 667721 | promoter | tumor | 0.88 | 11.79 |
| 253 | ENST00000583327.2 | ITGB4 | 17 | 73747894 | promoter | tumor | 0.12 | 32.84 |
| 254 | ENST00000586005.5 | SMIM22 | 16 | 4845422 | promoter | tumor | 0.00 | 103.03 |
| 255 | ENST00000587251.5 | LGALS3BP | 17 | 76976005 | promoter | tumor | 0.00 | 48.36 |
| 256 | ENST00000588605.5 | C19orf33 | 19 | 38794804 | promoter | tumor | 0.22 | 18.46 |
| 257 | ENST00000588606.5 | SMIM22 | 16 | 4845379 | promoter | tumor | 0.00 | 148.57 |
| 258 | ENST00000589378.5 | TJP3 | 19 | 3721735 | promoter | tumor | 0.13 | 11.74 |
| 259 | ENST00000591778.5 | LGALS3BP | 17 | 76976000 | promoter | tumor | 0.21 | 30.24 |
| 260 | ENST00000591795.1 | DENND1C | 19 | 6469662 | promoter | tumor | 0.56 | 11.26 |
| 261 | ENST00000594443.5 | FBL | 19 | 40337010 | promoter | tumor | 0.00 | 19.57 |
| 262 | ENST00000594605.5 | STAP2 | 19 | 4338874 | promoter | tumor | 0.06 | 27.33 |
| 263 | ENST00000595110.1 | FAM83E | 19 | 49118098 | promoter | tumor | 0.00 | 13.13 |
| 264 | ENST00000597153.5 | LGALS4 | 19 | 39303585 | promoter | tumor | 0.00 | 20.51 |
| 265 | ENST00000600229.5 | CRB3 | 19 | 6464290 | promoter | tumor | 0.08 | 20.31 |
| 266 | ENST00000600324.5 | STAP2 | 19 | 4338818 | promoter | tumor | 0.02 | 13.85 |
| 267 | ENST00000601623.5 | LSR | 19 | 35740250 | promoter | tumor | 0.21 | 24.39 |
| 268 | ENST00000605618.5 | LSR | 19 | 35739922 | promoter | tumor | 0.22 | 31.85 |
| 269 | ENST00000610552.4 | SLC2A8 | 9 | 130159417 | promoter | tumor | 0.65 | 11.38 |
| 270 | ENST00000612794.1 | GPX2 | 14 | 65409623 | promoter | tumor | 0.00 | 23.73 |
| 271 | ENST00000612809.4 | C8orf59 | 8 | 86132604 | promoter | tumor | 0.94 | 54.53 |
| 272 | ENST00000616154.1 | CDX1 | 5 | 149546453 | promoter | tumor | 0.00 | 138.07 |
| 273 | ENST00000616727.4 | MUC13 | 3 | 124653595 | promoter | tumor | 0.01 | 221.05 |
| 274 | ENST00000618855.4 | TMPRSS4 | 11 | 117947727 | promoter | tumor | 0.00 | 10.45 |
| 275 | ENST00000619895.4 | TMC4 | 19 | 54676865 | promoter | tumor | 0.35 | 33.05 |
| 276 | ENST00000620753.4 | EI24 | 11 | 125439354 | promoter | tumor | 0.63 | 12.17 |
| 277 | ENST00000008938.4 | PGLYRP1 | 19 | 46526323 | promoter | blood | 148.76 | 0.05 |
| 278 | ENST00000199708.2 | HBQ1 | 16 | 230452 | promoter | blood | 45.79 | 0.00 |
| 279 | ENST00000216338.8 | GZMH | 14 | 25078864 | promoter | blood | 15.58 | 0.99 |
| 280 | ENST00000221515.5 | RETN | 19 | 7733930 | promoter | blood | 72.49 | 0.19 |
| 281 | ENST00000221954.6 | CEACAM4 | 19 | 42133442 | promoter | blood | 32.81 | 0.13 |
| 282 | ENST00000225538.3 | P2RX1 | 17 | 3819794 | promoter | blood | 15.55 | 0.30 |
| 283 | ENST00000232014.8 | BCL6 | 3 | 187454357 | promoter | blood | 19.07 | 0.18 |
| 284 | ENST00000234347.9 | PRTN3 | 19 | 840960 | promoter | blood | 13.78 | 0.00 |
| 285 | ENST00000236826.7 | MMP8 | 11 | 102595685 | promoter | blood | 20.68 | 0.04 |
| 286 | ENST00000244709.8 | TREM1 | 6 | 41254457 | promoter | blood | 67.12 | 0.63 |
| 287 | ENST00000246115.4 | S1PR4 | 19 | 3178766 | promoter | blood | 127.44 | 0.94 |
| 288 | ENST00000246549.2 | FFAR2 | 19 | 35940617 | promoter | blood | 58.85 | 0.34 |
| 289 | ENST00000246657.2 | CCR7 | 17 | 38721724 | promoter | blood | 11.89 | 0.75 |
| 290 | ENST00000259396.8 | ORM1 | 9 | 117085336 | promoter | blood | 34.89 | 0.14 |
| 291 | ENST00000262407.5 | ITGA2B | 17 | 42466873 | promoter | blood | 12.34 | 0.04 |
| 292 | ENST00000264972.9 | ZAP70 | 2 | 98330023 | promoter | blood | 15.69 | 0.72 |
| 293 | ENST00000279452.10 | CD44 | 11 | 35198175 | promoter | blood | 10.76 | 0.00 |
| 294 | ENST00000281703.10 | GLT1D1 | 12 | 129338081 | promoter | blood | 22.75 | 0.02 |
| 295 | ENST00000290075.10 | SLC25A37 | 8 | 23386469 | promoter | blood | 45.08 | 0.87 |
| 296 | ENST00000292432.9 | HK3 | 5 | 176326333 | promoter | blood | 98.07 | 0.60 |
| 297 | ENST00000295683.2 | CXCR1 | 2 | 219031718 | promoter | blood | 355.57 | 0.18 |
| 298 | ENST00000296435.2 | CAMP | 3 | 48264837 | promoter | blood | 27.17 | 0.06 |
| 299 | ENST00000296487.8 | PPM1M | 3 | 52280186 | promoter | blood | 13.09 | 0.41 |
| 300 | ENST00000297239.10 | SYTL3 | 6 | 159082351 | promoter | blood | 10.79 | 0.54 |
| 301 | ENST00000299663.7 | CLEC4E | 12 | 8693559 | promoter | blood | 29.27 | 0.20 |
| 302 | ENST00000302017.3 | ZNF467 | 7 | 149470568 | promoter | blood | 25.10 | 0.54 |
| 303 | ENST00000303531.11 | PRKCB | 16 | 23847345 | promoter | blood | 16.08 | 0.36 |
| 304 | ENST00000303757.12 | LST1 | 6 | 31554977 | promoter | blood | 11.09 | 0.23 |
| 305 | ENST00000307564.8 | AKNA | 9 | 117156685 | promoter | blood | 11.95 | 0.38 |
| 306 | ENST00000310544.8 | PHOSPHO1 | 17 | 47307890 | promoter | blood | 30.92 | 0.08 |
| 307 | ENST00000318507.6 | CXCR2 | 2 | 218990727 | promoter | blood | 157.27 | 0.28 |
| 308 | ENST00000326165.10 | CD300LF | 17 | 72709117 | promoter | blood | 18.99 | 0.70 |
| 309 | ENST00000328118.7 | FMNL1 | 17 | 43299444 | promoter | blood | 11.80 | 0.41 |
| 310 | ENST00000329021.9 | NFAM1 | 22 | 42828401 | promoter | blood | 43.63 | 0.94 |
| 311 | ENST00000329410.3 | C16orf54 | 16 | 29757327 | promoter | blood | 24.31 | 0.49 |
| 312 | ENST00000332549.7 | IL1R2 | 2 | 102608306 | promoter | blood | 112.29 | 0.55 |
| 313 | ENST00000334475.10 | TREM1 | 6 | 41254420 | promoter | blood | 11.13 | 0.19 |
| 314 | ENST00000336577.8 | MMP25 | 16 | 3096682 | promoter | blood | 194.37 | 0.54 |
| 315 | ENST00000336906.4 | HBG2 | 11 | 5276041 | promoter | blood | 104.85 | 0.00 |
| 316 | ENST00000338372.6 | VSTM1 | 19 | 54567207 | promoter | blood | 10.71 | 0.00 |
| 317 | ENST00000342571.7 | MKNK1 | 1 | 47051643 | promoter | blood | 14.42 | 0.02 |
| 318 | ENST00000343534.9 | C1orf162 | 1 | 112016414 | promoter | blood | 37.68 | 0.74 |
| 319 | ENST00000346667.8 | IKZF1 | 7 | 50344378 | promoter | blood | 11.73 | 0.24 |
| 320 | ENST00000352818.8 | CD44 | 11 | 35160851 | promoter | blood | 14.11 | 0.00 |
| 321 | ENST00000354352.9 | SLC11A1 | 2 | 219246971 | promoter | blood | 111.80 | 0.63 |
| 322 | ENST00000355524.7 | FCAR | 19 | 55385736 | promoter | blood | 53.04 | 0.07 |
| 323 | ENST00000356815.3 | HBM | 16 | 215977 | promoter | blood | 163.82 | 0.00 |
| 324 | ENST00000356838.7 | TMEM71 | 8 | 133772828 | promoter | blood | 16.78 | 0.36 |
| 325 | ENST00000356864.3 | TNFRSF10C | 8 | 22960103 | promoter | blood | 108.86 | 0.56 |
| 326 | ENST00000357198.8 | DOK3 | 5 | 176936858 | promoter | blood | 66.68 | 0.74 |
| 327 | ENST00000357260.5 | FAM212B | 1 | 112282046 | promoter | blood | 12.23 | 0.67 |
| 328 | ENST00000358375.8 | OSCAR | 19 | 54604129 | promoter | blood | 18.93 | 0.28 |
| 329 | ENST00000367025.7 | TRAF3IP3 | 1 | 209929394 | promoter | blood | 17.52 | 0.64 |
| 330 | ENST00000367053.5 | CR1 | 1 | 207669502 | promoter | blood | 10.47 | 0.02 |
| 331 | ENST00000367535.7 | NCF2 | 1 | 183559716 | promoter | blood | 75.23 | 0.98 |
| 332 | ENST00000367568.4 | STX11 | 6 | 144471663 | promoter | blood | 24.42 | 0.79 |
| 333 | ENST00000367972.8 | FCGR2A | 1 | 161475220 | promoter | blood | 27.69 | 0.40 |
| 334 | ENST00000368015.1 | ARHGAP30 | 1 | 161039760 | promoter | blood | 20.61 | 0.58 |
| 335 | ENST00000368732.5 | S100A8 | 1 | 153363452 | promoter | blood | 53.91 | 0.00 |
| 336 | ENST00000368737.4 | S100A12 | 1 | 153348125 | promoter | blood | 1099.02 | 0.31 |
| 337 | ENST00000371806.3 | FCN1 | 9 | 137809809 | promoter | blood | 379.64 | 0.88 |
| 338 | ENST00000373103.5 | CSF3R | 1 | 36948879 | promoter | blood | 61.20 | 0.04 |
| 339 | ENST00000373925.5 | THEMIS2 | 1 | 28199055 | promoter | blood | 12.15 | 0.53 |
| 340 | ENST00000374005.7 | FGR | 1 | 27961788 | promoter | blood | 32.42 | 0.29 |
| 341 | ENST00000374163.5 | RPS6KA1 | 1 | 26872333 | promoter | blood | 17.41 | 0.87 |
| 342 | ENST00000375448.4 | PADI4 | 1 | 17634692 | promoter | blood | 95.04 | 0.03 |
| 343 | ENST00000375862.6 | HCK | 20 | 30640000 | promoter | blood | 20.11 | 0.09 |
| 344 | ENST00000376670.7 | GATA1 | X | 48644962 | promoter | blood | 11.17 | 0.01 |
| 345 | ENST00000377497.7 | RASGRP2 | 11 | 64512306 | promoter | blood | 48.02 | 0.55 |
| 346 | ENST00000378023.8 | FAM65B | 6 | 24877583 | promoter | blood | 15.67 | 0.00 |
| 347 | ENST00000379400.7 | RASSF2 | 20 | 4804291 | promoter | blood | 13.75 | 0.82 |
| 348 | ENST00000380299.3 | HBD | 11 | 5255878 | promoter | blood | 138.84 | 0.07 |
| 349 | ENST00000381112.7 | CCM2 | 7 | 45067233 | promoter | blood | 17.78 | 0.00 |
| 350 | ENST00000381153.7 | C11orf21 | 11 | 2323290 | promoter | blood | 11.33 | 0.09 |
| 351 | ENST00000381603.7 | SIRPB1 | 20 | 1600642 | promoter | blood | 26.01 | 0.00 |
| 352 | ENST00000381605.8 | SIRPB1 | 20 | 1600655 | promoter | blood | 24.72 | 0.12 |
| 353 | ENST00000391750.5 | LILRB3 | 19 | 54727378 | promoter | blood | 65.61 | 0.41 |
| 354 | ENST00000391761.5 | OSCAR | 19 | 54604083 | promoter | blood | 35.99 | 0.46 |
| 355 | ENST00000393450.5 | MYL4 | 17 | 45286714 | promoter | blood | 22.77 | 0.00 |
| 356 | ENST00000393847.5 | DPEP2 | 16 | 68033356 | promoter | blood | 24.04 | 0.44 |
| 357 | ENST00000394991.7 | SNCA | 4 | 90758379 | promoter | blood | 32.19 | 0.35 |
| 358 | ENST00000397147.6 | NCF4 | 22 | 37257030 | promoter | blood | 154.32 | 0.92 |
| 359 | ENST00000398421.6 | NCF1 | 7 | 74188358 | promoter | blood | 11.04 | 0.04 |
| 360 | ENST00000399173.5 | FGR | 1 | 27952751 | promoter | blood | 101.05 | 0.26 |
| 361 | ENST00000399753.2 | MSRB1 | 16 | 1993196 | promoter | blood | 11.22 | 0.15 |
| 362 | ENST00000409220.5 | ARHGAP25 | 2 | 69001933 | promoter | blood | 23.89 | 0.31 |
| 363 | ENST00000409930.3 | IL1RN | 2 | 113885138 | promoter | blood | 21.18 | 0.85 |
| 364 | ENST00000413580.5 | PHOSPHO1 | 17 | 47308128 | promoter | blood | 42.76 | 0.05 |
| 365 | ENST00000416215.6 | PTPN6 | 12 | 7060519 | promoter | blood | 12.72 | 0.88 |
| 366 | ENST00000418089.5 | NCF2 | 1 | 183559739 | promoter | blood | 12.55 | 0.12 |
| 367 | ENST00000419510.6 | BCL6 | 3 | 187454876 | promoter | blood | 45.28 | 0.23 |
| 368 | ENST00000422400.6 | VNN2 | 6 | 133079022 | promoter | blood | 14.59 | 0.10 |
| 369 | ENST00000423345.4 | PRAM1 | 19 | 8567996 | promoter | blood | 25.32 | 0.30 |
| 370 | ENST00000425428.6 | CD44 | 11 | 35160729 | promoter | blood | 17.51 | 0.00 |
| 371 | ENST00000433194.6 | CDK5RAP2 | 9 | 123165730 | promoter | blood | 22.55 | 0.52 |
| 372 | ENST00000434472.6 | CD44 | 11 | 35160840 | promoter | blood | 11.65 | 0.58 |
| 373 | ENST00000441002.1 | IL1R2 | 2 | 102624977 | promoter | blood | 16.30 | 0.00 |
| 374 | ENST00000442111.6 | GLT1D1 | 12 | 129338039 | promoter | blood | 20.30 | 0.01 |
| 375 | ENST00000445347.1 | LILRB3 | 19 | 54726959 | promoter | blood | 49.26 | 0.10 |
| 376 | ENST00000445961.5 | RPS9 | 19 | 54704740 | promoter | blood | 14.72 | 0.89 |
| 377 | ENST00000447110.5 | PIK3R5 | 17 | 8869029 | promoter | blood | 12.89 | 0.29 |
| 378 | ENST00000449131.6 | BEST1 | 11 | 61717842 | promoter | blood | 13.35 | 0.54 |
| 379 | ENST00000454703.6 | ACSL1 | 4 | 185747273 | promoter | blood | 35.07 | 0.78 |
| 380 | ENST00000460442.5 | ZDHHC19 | 3 | 195925781 | promoter | blood | 22.43 | 0.04 |
| 381 | ENST00000464431.1 | ABTB1 | 3 | 127394329 | promoter | blood | 20.22 | 0.70 |
| 382 | ENST00000464465.6 | CSF3R | 1 | 36937075 | promoter | blood | 259.11 | 0.50 |
| 383 | ENST00000465814.5 | ARAP1 | 11 | 72433133 | promoter | blood | 10.09 | 0.58 |
| 384 | ENST00000465984.5 | SLC11A1 | 2 | 219246911 | promoter | blood | 10.54 | 0.03 |
| 385 | ENST00000467786.1 | FGD3 | 9 | 95737532 | promoter | blood | 43.15 | 0.29 |
| 386 | ENST00000474085.5 | IL1R2 | 2 | 102638525 | promoter | blood | 24.35 | 0.04 |
| 387 | ENST00000475225.5 | SLC11A1 | 2 | 219247025 | promoter | blood | 37.49 | 0.19 |
| 388 | ENST00000475226.1 | HBB | 11 | 5248053 | promoter | blood | 2709.48 | 0.00 |
| 389 | ENST00000475472.5 | FGR | 1 | 27961645 | promoter | blood | 49.95 | 0.18 |
| 390 | ENST00000477801.1 | S100A8 | 1 | 153363547 | promoter | blood | 161.47 | 0.07 |
| 391 | ENST00000480395.5 | TRIM22 | 11 | 5717722 | promoter | blood | 17.36 | 0.70 |
| 392 | ENST00000481568.2 | C10orf54 | 10 | 73521701 | promoter | blood | 31.27 | 0.90 |
| 393 | ENST00000483750.5 | WAS | X | 48542217 | promoter | blood | 34.49 | 0.17 |
| 394 | ENST00000484747.5 | ZNF467 | 7 | 149470308 | promoter | blood | 14.36 | 0.44 |
| 395 | ENST00000485743.1 | HBB | 11 | 5248302 | promoter | blood | 128.52 | 0.00 |
| 396 | ENST00000485928.5 | PHC2 | 1 | 33815412 | promoter | blood | 11.05 | 0.73 |
| 397 | ENST00000486097.1 | NCF1 | 7 | 74197786 | promoter | blood | 12.51 | 0.06 |
| 398 | ENST00000487540.6 | CSF3R | 1 | 36937988 | promoter | blood | 47.30 | 0.17 |
| 399 | ENST00000488171.5 | CD55 | 1 | 207495032 | promoter | blood | 25.43 | 0.43 |
| 400 | ENST00000488945.5 | GNLY | 2 | 85912298 | promoter | blood | 11.68 | 0.09 |
| 401 | ENST00000489358.5 | NAMPT | 7 | 105925396 | promoter | blood | 30.07 | 0.00 |
| 402 | ENST00000489551.5 | CSF3R | 1 | 36948535 | promoter | blood | 106.01 | 0.10 |
| 403 | ENST00000491955.5 | MPP1 | X | 154011509 | promoter | blood | 10.84 | 0.42 |
| 404 | ENST00000492413.5 | SLC11A1 | 2 | 219247010 | promoter | blood | 17.89 | 0.00 |
| 405 | ENST00000493242.1 | LILRB2 | 19 | 54784952 | promoter | blood | 22.65 | 0.13 |
| 406 | ENST00000494185.1 | LMNB1 | 5 | 126147405 | promoter | blood | 10.53 | 0.00 |
| 407 | ENST00000496823.1 | BCL6 | 3 | 187463247 | promoter | blood | 21.33 | 0.20 |
| 408 | ENST00000497259.5 | ARHGAP25 | 2 | 69034363 | promoter | blood | 25.49 | 0.72 |
| 409 | ENST00000497920.1 | ARID5A | 2 | 97213127 | promoter | blood | 11.90 | 0.30 |
| 410 | ENST00000509314.5 | FBXL5 | 4 | 15661487 | promoter | blood | 10.24 | 0.00 |
| 411 | ENST00000509339.1 | MXD3 | 5 | 176735063 | promoter | blood | 30.07 | 0.08 |
| 412 | ENST00000510784.6 | FAM65B | 6 | 25042396 | promoter | blood | 61.47 | 0.19 |
| 413 | ENST00000513001.5 | ACSL1 | 4 | 185680030 | promoter | blood | 17.65 | 0.10 |
| 414 | ENST00000517558.1 | TNFRSF10C | 8 | 22960434 | promoter | blood | 45.94 | 0.13 |
| 415 | ENST00000519192.1 | SLC25A37 | 8 | 23386488 | promoter | blood | 357.35 | 0.55 |
| 416 | ENST00000520174.5 | DMTN | 8 | 21914416 | promoter | blood | 16.02 | 0.00 |
| 417 | ENST00000520553.5 | HCK | 20 | 30640045 | promoter | blood | 87.74 | 0.73 |
| 418 | ENST00000520654.1 | SLC25A37 | 8 | 23386566 | promoter | blood | 18.30 | 0.77 |
| 419 | ENST00000523022.5 | CA1 | 8 | 86290342 | promoter | blood | 40.92 | 0.11 |
| 420 | ENST00000523829.5 | TMEM71 | 8 | 133772807 | promoter | blood | 11.05 | 0.22 |
| 421 | ENST00000525270.5 | VNN2 | 6 | 133084586 | promoter | blood | 38.37 | 0.12 |
| 422 | ENST00000526980.5 | CSF3R | 1 | 36948500 | promoter | blood | 184.80 | 0.10 |
| 423 | ENST00000527146.1 | IFITM2 | 11 | 308320 | promoter | blood | 18.56 | 0.63 |
| 424 | ENST00000528780.5 | IFITM1 | 11 | 313506 | promoter | blood | 47.88 | 0.31 |
| 425 | ENST00000529635.5 | TBC1D10C | 11 | 67171600 | promoter | blood | 16.89 | 0.34 |
| 426 | ENST00000533968.1 | SPI1 | 11 | 47400038 | promoter | blood | 12.90 | 0.22 |
| 427 | ENST00000534862.5 | HCK | 20 | 30639991 | promoter | blood | 16.31 | 0.23 |
| 428 | ENST00000535669.6 | CD37 | 19 | 49838684 | promoter | blood | 33.70 | 0.19 |
| 429 | ENST00000539932.5 | SLC11A1 | 2 | 219246926 | promoter | blood | 50.32 | 0.02 |
| 430 | ENST00000540998.5 | CDC42SE1 | 1 | 151032125 | promoter | blood | 10.52 | 0.00 |
| 431 | ENST00000542481.1 | ATG16L2 | 11 | 72534940 | promoter | blood | 25.18 | 0.37 |
| 432 | ENST00000542590.1 | TBC1D10C | 11 | 67171660 | promoter | blood | 43.95 | 0.92 |
| 433 | ENST00000543576.5 | DENND1C | 19 | 6481798 | promoter | blood | 12.06 | 0.00 |
| 434 | ENST00000544232.5 | TMEM91 | 19 | 41882645 | promoter | blood | 12.57 | 0.20 |
| 435 | ENST00000544665.7 | ITGAM | 16 | 31271315 | promoter | blood | 56.38 | 0.99 |
| 436 | ENST00000546704.1 | ARHGAP9 | 12 | 57868923 | promoter | blood | 19.20 | 0.13 |
| 437 | ENST00000550399.5 | ARHGAP9 | 12 | 57868717 | promoter | blood | 14.44 | 0.45 |
| 438 | ENST00000552370.5 | TMBIM6 | 12 | 50144403 | promoter | blood | 26.69 | 0.00 |
| 439 | ENST00000553070.5 | NFE2 | 12 | 54694799 | promoter | blood | 23.61 | 0.00 |
| 440 | ENST00000558012.5 | PSTPIP1 | 15 | 77287426 | promoter | blood | 26.47 | 0.31 |
| 441 | ENST00000559750.5 | PSTPIP1 | 15 | 77287513 | promoter | blood | 16.39 | 0.12 |
| 442 | ENST00000564662.1 | COTL1 | 16 | 84651681 | promoter | blood | 10.13 | 0.81 |
| 443 | ENST00000564905.1 | XPO6 | 16 | 28145246 | promoter | blood | 12.98 | 0.47 |
| 444 | ENST00000568763.1 | CORO1A | 16 | 30194926 | promoter | blood | 45.17 | 0.63 |
| 445 | ENST00000570106.6 | SIGLEC5 | 19 | 52133588 | promoter | blood | 40.53 | 0.18 |
| 446 | ENST00000572782.1 | ARRB2 | 17 | 4617846 | promoter | blood | 16.63 | 0.33 |
| 447 | ENST00000576628.1 | ACAP1 | 17 | 7239916 | promoter | blood | 20.20 | 0.18 |
| 448 | ENST00000577894.1 | EVI2B | 17 | 29641123 | promoter | blood | 14.34 | 0.00 |
| 449 | ENST00000578067.5 | LIMD2 | 17 | 61777468 | promoter | blood | 10.86 | 0.22 |
| 450 | ENST00000578402.5 | LIMD2 | 17 | 61778045 | promoter | blood | 31.49 | 0.77 |
| 451 | ENST00000585901.6 | TYROBP | 19 | 36399149 | promoter | blood | 13.40 | 0.19 |
| 452 | ENST00000586946.1 | TYROBP | 19 | 36399197 | promoter | blood | 23.56 | 0.61 |
| 453 | ENST00000587259.5 | VMP1 | 17 | 57807076 | promoter | blood | 10.58 | 0.05 |
| 454 | ENST00000587856.1 | FMNL1 | 17 | 43311251 | promoter | blood | 22.20 | 0.28 |
| 455 | ENST00000588673.3 | OAZ1 | 19 | 2270290 | promoter | blood | 24.28 | 0.69 |
| 456 | ENST00000589614.5 | TREM1 | 6 | 41254403 | promoter | blood | 31.20 | 0.54 |
| 457 | ENST00000589900.5 | ICAM3 | 19 | 10450305 | promoter | blood | 21.24 | 0.00 |
| 458 | ENST00000592860.2 | CFD | 19 | 859664 | promoter | blood | 16.53 | 0.85 |
| 459 | ENST00000595042.5 | FPR1 | 19 | 52255146 | promoter | blood | 63.93 | 0.18 |
| 460 | ENST00000595217.1 | NKG7 | 19 | 51875955 | promoter | blood | 33.26 | 0.52 |
| 461 | ENST00000595325.5 | MYO1F | 19 | 8642322 | promoter | blood | 13.66 | 0.09 |
| 462 | ENST00000595636.1 | GMFG | 19 | 39826664 | promoter | blood | 10.44 | 0.68 |
| 463 | ENST00000595725.5 | CD37 | 19 | 49838657 | promoter | blood | 20.04 | 0.12 |
| 464 | ENST00000595840.1 | LRRC25 | 19 | 18508421 | promoter | blood | 36.84 | 0.88 |
| 465 | ENST00000596426.5 | CD37 | 19 | 49838691 | promoter | blood | 55.55 | 0.57 |
| 466 | ENST00000596764.5 | VAV1 | 19 | 6772739 | promoter | blood | 12.25 | 0.59 |
| 467 | ENST00000597852.5 | CD37 | 19 | 49838675 | promoter | blood | 13.98 | 0.20 |
| 468 | ENST00000598034.5 | GMFG | 19 | 39826646 | promoter | blood | 43.77 | 0.45 |
| 469 | ENST00000599180.2 | FFAR2 | 19 | 35939203 | promoter | blood | 24.79 | 0.00 |
| 470 | ENST00000599716.5 | SHKBP1 | 19 | 41082793 | promoter | blood | 10.66 | 0.22 |
| 471 | ENST00000600626.1 | C5AR2 | 19 | 47840371 | promoter | blood | 12.99 | 0.11 |
| 472 | ENST00000600972.1 | JUND | 19 | 18391739 | promoter | blood | 17.49 | 0.60 |
| 473 | ENST00000602185.5 | GMFG | 19 | 39826645 | promoter | blood | 14.09 | 0.00 |
| 474 | ENST00000602569.1 | IFITM2 | 11 | 308217 | promoter | blood | 3175.79 | 0.00 |
| 475 | ENST00000605039.5 | BIN2 | 12 | 51717938 | promoter | blood | 11.02 | 0.11 |
| 476 | ENST00000611028.2 | FOLR3 | 11 | 71846798 | promoter | blood | 11.13 | 0.00 |
| 477 | ENST00000612844.4 | FOLR3 | 11 | 71846756 | promoter | blood | 13.11 | 0.00 |
| 478 | ENST00000615439.4 | RASGRP4 | 19 | 38916945 | promoter | blood | 10.11 | 0.00 |
| 479 | ENST00000615825.1 | MME | 3 | 154801957 | promoter | blood | 12.37 | 0.88 |
| 480 | ENST00000616356.4 | FCN1 | 9 | 137809723 | promoter | blood | 16.99 | 0.07 |
| 481 | ENST00000618265.4 | CD177 | 19 | 43857811 | promoter | blood | 222.54 | 0.73 |
| 482 | ENST00000620541.4 | RIN3 | 14 | 92980137 | promoter | blood | 17.39 | 0.84 |
| 483 | ENST00000620888.4 | HBG2 | 11 | 5276011 | promoter | blood | 161.14 | 0.00 |
| 484 | ENST00000003084.10 | CFTR | 7 | 117120201 | junction | tumor | 0.00 | 30.20 |
| 485 | ENST00000027335.7 | CDH17 | 8 | 95220711 | junction | tumor | 0.00 | 149.83 |
| 486 | ENST00000161006.7 | PRSS22 | 16 | 2908024 | junction | tumor | 0.00 | 10.29 |
| 487 | ENST00000162330.9 | BCAR1 | 16 | 75285369 | junction | tumor | 0.00 | 16.86 |
| 488 | ENST00000201586.6 | SULT2B1 | 19 | 49055580 | junction | tumor | 0.03 | 16.67 |
| 489 | ENST00000211314.4 | TMEM14A | 6 | 52536043 | junction | tumor | 0.67 | 25.17 |
| 490 | ENST00000215582.7 | MISP | 19 | 751171 | junction | tumor | 0.00 | 116.54 |
| 491 | ENST00000215743.7 | MMP11 | 22 | 24115165 | junction | tumor | 0.08 | 42.41 |
| 492 | ENST00000216968.4 | PROCR | 20 | 33760027 | junction | tumor | 0.36 | 28.48 |
| 493 | ENST00000221992.10 | CEACAM5 | 19 | 42212714 | junction | tumor | 0.00 | 1235.90 |
| 494 | ENST00000223271.7 | RARRES2 | 7 | 150038667 | junction | tumor | 0.41 | 46.30 |
| 495 | ENST00000226230.7 | TMEM97 | 17 | 26646391 | junction | tumor | 0.39 | 20.84 |
| 496 | ENST00000226760.5 | WFS1 | 4 | 6271741 | junction | tumor | 0.35 | 15.01 |
| 497 | ENST00000227868.8 | PDHX | 11 | 34938362 | junction | tumor | 0.99 | 13.52 |
| 498 | ENST00000228916.6 | SCNN1A | 12 | 6484671 | junction | tumor | 0.05 | 32.36 |
| 499 | ENST00000230588.8 | MEP1A | 6 | 46761204 | junction | tumor | 0.00 | 21.95 |
| 500 | ENST00000232458.9 | ECT2 | 3 | 172468647 | junction | tumor | 0.28 | 12.07 |
| 501 | ENST00000245451.8 | BMP4 | 14 | 54423268 | junction | tumor | 0.00 | 16.43 |
| 502 | ENST00000245907.10 | C3 | 19 | 6720527 | junction | tumor | 0.37 | 15.08 |
| 503 | ENST00000250405.9 | BCL2L2 | 14 | 23776103 | junction | tumor | 1.00 | 11.63 |
| 504 | ENST00000254260.7 | RHPN2 | 19 | 33555690 | junction | tumor | 0.03 | 23.49 |
| 505 | ENST00000255681.6 | MACROD1 | 11 | 63933169 | junction | tumor | 0.54 | 22.18 |
| 506 | ENST00000256585.9 | REG4 | 1 | 120354032 | junction | tumor | 0.09 | 37.82 |
| 507 | ENST00000256951.9 | EMP1 | 12 | 13349806 | junction | tumor | 0.39 | 14.17 |
| 508 | ENST00000260227.4 | MMP7 | 11 | 102401324 | junction | tumor | 0.00 | 29.38 |
| 509 | ENST00000261769.9 | CDH1 | 16 | 68771366 | junction | tumor | 0.00 | 123.30 |
| 510 | ENST00000262429.8 | ATP2C2 | 16 | 84402320 | junction | tumor | 0.34 | 16.47 |
| 511 | ENST00000262753.8 | POF1B | X | 84634644 | junction | tumor | 0.02 | 40.41 |
| 512 | ENST00000263629.8 | MTIF2 | 2 | 55496305 | junction | tumor | 0.85 | 13.76 |
| 513 | ENST00000263735.8 | EPCAM | 2 | 47596720 | junction | tumor | 0.11 | 594.54 |
| 514 | ENST00000263895.8 | RND3 | 2 | 151344063 | junction | tumor | 0.00 | 10.29 |
| 515 | ENST00000264144.4 | LAMC2 | 1 | 183155566 | junction | tumor | 0.01 | 31.46 |
| 516 | ENST00000264748.6 | FGFRL1 | 4 | 1006352 | junction | tumor | 0.82 | 17.03 |
| 517 | ENST00000266980.8 | SLC39A5 | 12 | 56624657 | junction | tumor | 0.00 | 20.87 |
| 518 | ENST00000267101.7 | ERBB3 | 12 | 56474166 | junction | tumor | 0.00 | 40.98 |
| 519 | ENST00000267814.13 | SORD | 15 | 45315547 | junction | tumor | 0.93 | 13.70 |
| 520 | ENST00000267996.11 | TPM1 | 15 | 63335142 | junction | tumor | 0.87 | 32.98 |
| 521 | ENST00000269571.9 | ERBB2 | 17 | 37856564 | junction | tumor | 0.00 | 11.47 |
| 522 | ENST00000270560.3 | TM4SF5 | 17 | 4675394 | junction | tumor | 0.00 | 18.39 |
| 523 | ENST00000271064.11 | TINAGL1 | 1 | 32042196 | junction | tumor | 0.10 | 42.75 |
| 524 | ENST00000278559.7 | CAPN5 | 11 | 76778141 | junction | tumor | 0.52 | 27.31 |
| 525 | ENST00000278937.6 | MPZL2 | 11 | 118134811 | junction | tumor | 0.37 | 17.05 |
| 526 | ENST00000288937.6 | MRPL17 | 11 | 6704354 | junction | tumor | 0.85 | 16.44 |
| 527 | ENST00000290130.3 | MIS18A | 21 | 33650992 | junction | tumor | 0.49 | 12.76 |
| 528 | ENST00000290913.7 | CHCHD6 | 3 | 126423242 | junction | tumor | 0.65 | 15.07 |
| 529 | ENST00000291525.11 | TFF3 | 21 | 43735403 | junction | tumor | 0.00 | 209.02 |
| 530 | ENST00000292401.8 | AZGP1 | 7 | 99573568 | junction | tumor | 0.14 | 45.24 |
| 531 | ENST00000292408.8 | FGFR4 | 5 | 176514078 | junction | tumor | 0.07 | 25.86 |
| 532 | ENST00000295092.2 | FAM84A | 2 | 14773061 | junction | tumor | 0.00 | 13.18 |
| 533 | ENST00000296129.5 | CDCP1 | 3 | 45187698 | junction | tumor | 0.11 | 15.27 |
| 534 | ENST00000296252.8 | LIPH | 3 | 185270211 | junction | tumor | 0.03 | 12.23 |
| 535 | ENST00000296424.8 | BDH2 | 4 | 104020940 | junction | tumor | 0.99 | 14.16 |
| 536 | ENST00000296666.12 | PRRC1 | 5 | 126853468 | junction | tumor | 0.95 | 11.62 |
| 537 | ENST00000297205.6 | STEAP1 | 7 | 89783857 | junction | tumor | 0.00 | 14.91 |
| 538 | ENST00000299610.4 | MFAP4 | 17 | 19290463 | junction | tumor | 0.39 | 15.19 |
| 539 | ENST00000299714.7 | SEC11C | 18 | 56807267 | junction | tumor | 0.62 | 11.38 |
| 540 | ENST00000300119.7 | MYO1A | 12 | 57443671 | junction | tumor | 0.00 | 15.11 |
| 541 | ENST00000300283.10 | CKMT1B | 15 | 43885469 | junction | tumor | 0.00 | 35.88 |
| 542 | ENST00000300557.2 | PRR15L | 17 | 46035023 | junction | tumor | 0.03 | 96.21 |
| 543 | ENST00000301395.7 | GGT6 | 17 | 4463677 | junction | tumor | 0.00 | 16.81 |
| 544 | ENST00000301887.8 | BATF2 | 11 | 64764348 | junction | tumor | 0.43 | 11.12 |
| 545 | ENST00000307078.9 | AXIN2 | 17 | 63557568 | junction | tumor | 0.09 | 14.10 |
| 546 | ENST00000308302.3 | GOLT1A | 1 | 204183010 | junction | tumor | 0.03 | 12.22 |
| 547 | ENST00000308831.6 | RHOD | 11 | 66824505 | junction | tumor | 0.07 | 10.41 |
| 548 | ENST00000310398.6 | AGR3 | 7 | 16921568 | junction | tumor | 0.00 | 33.08 |
| 549 | ENST00000310706.9 | JUP | 17 | 39942840 | junction | tumor | 0.87 | 86.21 |
| 550 | ENST00000310836.10 | UGT8 | 4 | 115520130 | junction | tumor | 0.04 | 14.08 |
| 551 | ENST00000311160.13 | TNS3 | 7 | 47621649 | junction | tumor | 0.68 | 16.18 |
| 552 | ENST00000311381.7 | C6orf203 | 6 | 107349475 | junction | tumor | 0.93 | 14.55 |
| 553 | ENST00000311620.6 | ANKS4B | 16 | 21245222 | junction | tumor | 0.00 | 10.61 |
| 554 | ENST00000316673.8 | HNF4A | 20 | 42984493 | junction | tumor | 0.00 | 10.78 |
| 555 | ENST00000317508.10 | PRSS8 | 16 | 31146735 | junction | tumor | 0.11 | 143.22 |
| 556 | ENST00000318024.8 | USH1C | 11 | 17565819 | junction | tumor | 0.00 | 46.62 |
| 557 | ENST00000318407.4 | BOK | 2 | 242498408 | junction | tumor | 0.57 | 13.75 |
| 558 | ENST00000318443.9 | CD276 | 15 | 73976801 | junction | tumor | 0.00 | 11.45 |
| 559 | ENST00000318683.6 | B3GNT3 | 19 | 17906015 | junction | tumor | 0.00 | 57.75 |
| 560 | ENST00000319838.9 | GPR35 | 2 | 241544987 | junction | tumor | 0.31 | 30.59 |
| 561 | ENST00000322536.7 | DDX10 | 11 | 108536066 | junction | tumor | 0.75 | 10.84 |
| 562 | ENST00000324038.6 | FAM101A | 12 | 124773916 | junction | tumor | 0.00 | 10.08 |
| 563 | ENST00000325307.11 | HMGB3 | X | 150151849 | junction | tumor | 0.30 | 14.09 |
| 564 | ENST00000325568.9 | IL32 | 16 | 3115495 | junction | tumor | 0.93 | 14.52 |
| 565 | ENST00000326587.11 | MAGED1 | X | 51636851 | junction | tumor | 0.60 | 16.14 |
| 566 | ENST00000329875.12 | PYCR1 | 17 | 79894624 | junction | tumor | 0.12 | 36.33 |
| 567 | ENST00000331595.8 | BGN | X | 152760571 | junction | tumor | 0.60 | 74.92 |
| 568 | ENST00000332149.9 | TMPRSS2 | 21 | 42880008 | junction | tumor | 0.02 | 43.57 |
| 569 | ENST00000333090.4 | TSKU | 11 | 76494448 | junction | tumor | 0.00 | 17.21 |
| 570 | ENST00000334047.11 | F3 | 1 | 95007093 | junction | tumor | 0.08 | 13.07 |
| 571 | ENST00000334869.8 | LGMN | 14 | 93214834 | junction | tumor | 0.94 | 45.73 |
| 572 | ENST00000337682.8 | FAM60A | 12 | 31478958 | junction | tumor | 0.97 | 25.73 |
| 573 | ENST00000338660.5 | VWA1 | 1 | 1371201 | junction | tumor | 0.06 | 21.82 |
| 574 | ENST00000339276.5 | SFN | 1 | 27190948 | junction | tumor | 0.60 | 113.82 |
| 575 | ENST00000340556.10 | MORN2 | 2 | 39103251 | junction | tumor | 0.33 | 11.41 |
| 576 | ENST00000344616.3 | S100A14 | 1 | 153588771 | junction | tumor | 0.00 | 143.55 |
| 577 | ENST00000350051.7 | BIRC5 | 17 | 76210508 | junction | tumor | 0.63 | 18.32 |
| 578 | ENST00000352551.9 | UBE2C | 20 | 44441435 | junction | tumor | 0.00 | 10.99 |
| 579 | ENST00000354624.5 | HKDC1 | 10 | 70980254 | junction | tumor | 0.14 | 20.07 |
| 580 | ENST00000354900.7 | LSR | 19 | 35740034 | junction | tumor | 0.75 | 31.22 |
| 581 | ENST00000355097.6 | ENTPD2 | 9 | 139948333 | junction | tumor | 0.03 | 10.14 |
| 582 | ENST00000355808.9 | PDHA1 | X | 19362212 | junction | tumor | 0.25 | 16.21 |
| 583 | ENST00000355899.7 | PLS3 | X | 114795587 | junction | tumor | 0.00 | 12.05 |
| 584 | ENST00000356509.7 | GMNN | 6 | 24775340 | junction | tumor | 0.57 | 11.97 |
| 585 | ENST00000356762.7 | CRB3 | 19 | 6464361 | junction | tumor | 0.00 | 12.29 |
| 586 | ENST00000357166.10 | ZDHHC9 | X | 128977672 | junction | tumor | 0.29 | 20.11 |
| 587 | ENST00000357602.7 | IPO5 | 13 | 98612657 | junction | tumor | 0.80 | 15.40 |
| 588 | ENST00000358432.7 | EPHA2 | 1 | 16482343 | junction | tumor | 0.03 | 23.85 |
| 589 | ENST00000358813.4 | CCL20 | 2 | 228678703 | junction | tumor | 0.00 | 33.26 |
| 590 | ENST00000358867.10 | TMEM126B | 11 | 85339732 | junction | tumor | 0.47 | 17.68 |
| 591 | ENST00000360325.11 | CLDN7 | 17 | 7165140 | junction | tumor | 0.09 | 133.65 |
| 592 | ENST00000360605.8 | URI1 | 19 | 30414661 | junction | tumor | 0.44 | 12.86 |
| 593 | ENST00000360760.9 | SPATS2L | 2 | 201171324 | junction | tumor | 0.73 | 32.04 |
| 594 | ENST00000360779.3 | SDCBP2 | 20 | 1309729 | junction | tumor | 0.00 | 17.84 |
| 595 | ENST00000361084.9 | RAB25 | 1 | 156031234 | junction | tumor | 0.07 | 131.50 |
| 596 | ENST00000361852.8 | EPS8L3 | 1 | 110306444 | junction | tumor | 0.00 | 26.64 |
| 597 | ENST00000361901.6 | CALD1 | 7 | 134464500 | junction | tumor | 0.42 | 33.93 |
| 598 | ENST00000366787.7 | THBS2 | 6 | 169654085 | junction | tumor | 0.07 | 14.23 |
| 599 | ENST00000366999.8 | NEK2 | 1 | 211848726 | junction | tumor | 0.16 | 11.93 |
| 600 | ENST00000367274.8 | UBE2T | 1 | 202311023 | junction | tumor | 0.63 | 29.13 |
| 601 | ENST00000367283.7 | ELF3 | 1 | 201979948 | junction | tumor | 0.07 | 135.46 |
| 602 | ENST00000367284.9 | ELF3 | 1 | 201979834 | junction | tumor | 0.02 | 50.55 |
| 603 | ENST00000367313.4 | LAD1 | 1 | 201368538 | junction | tumor | 0.05 | 100.09 |
| 604 | ENST00000367976.3 | CTGF | 6 | 132272247 | junction | tumor | 0.32 | 44.18 |
| 605 | ENST00000368408.3 | EFNA3 | 1 | 155051545 | junction | tumor | 0.36 | 18.08 |
| 606 | ENST00000368554.8 | PRAP1 | 10 | 135122967 | junction | tumor | 0.00 | 32.41 |
| 607 | ENST00000369406.7 | HMGCS2 | 1 | 120311364 | junction | tumor | 0.00 | 17.63 |
| 608 | ENST00000370481.8 | GBP3 | 1 | 89488367 | junction | tumor | 0.66 | 12.56 |
| 609 | ENST00000370828.3 | GPC4 | X | 132548834 | junction | tumor | 0.03 | 13.27 |
| 610 | ENST00000371221.3 | ALDH18A1 | 10 | 97416354 | junction | tumor | 0.04 | 29.55 |
| 611 | ENST00000372486.5 | NTMT1 | 9 | 132371457 | junction | tumor | 0.00 | 14.12 |
| 612 | ENST00000372966.7 | NOX1 | X | 100129084 | junction | tumor | 0.00 | 48.52 |
| 613 | ENST00000373255.8 | H2AFY2 | 10 | 71812756 | junction | tumor | 0.42 | 12.60 |
| 614 | ENST00000373290.6 | TSPAN15 | 10 | 71211446 | junction | tumor | 0.30 | 60.96 |
| 615 | ENST00000373669.6 | PIN4 | X | 71401678 | junction | tumor | 0.87 | 13.81 |
| 616 | ENST00000373699.5 | PPIL1 | 6 | 36842493 | junction | tumor | 0.85 | 17.26 |
| 617 | ENST00000373944.7 | ZWINT | 10 | 58120957 | junction | tumor | 0.38 | 16.27 |
| 618 | ENST00000374183.4 | BSPRY | 9 | 116112060 | junction | tumor | 0.22 | 15.59 |
| 619 | ENST00000374214.3 | UQCC2 | 6 | 33679326 | junction | tumor | 1.00 | 17.77 |
| 620 | ENST00000374837.7 | MAP1LC3A | 20 | 33134720 | junction | tumor | 0.87 | 16.45 |
| 621 | ENST00000375431.8 | GRTP1 | 13 | 114018335 | junction | tumor | 0.05 | 18.16 |
| 622 | ENST00000376569.7 | DDR1 | 6 | 30851922 | junction | tumor | 0.08 | 11.00 |
| 623 | ENST00000376652.8 | ENTPD6 | 20 | 25176503 | junction | tumor | 0.44 | 14.06 |
| 624 | ENST00000378115.2 | ARHGEF35 | 7 | 143892631 | junction | tumor | 0.10 | 13.16 |
| 625 | ENST00000378378.8 | ARHGEF16 | 1 | 3371375 | junction | tumor | 0.00 | 12.12 |
| 626 | ENST00000378427.5 | FAM213B | 1 | 2518387 | junction | tumor | 0.59 | 12.06 |
| 627 | ENST00000379046.6 | NQO1 | 16 | 69760336 | junction | tumor | 0.07 | 39.35 |
| 628 | ENST00000379715.9 | EEF1E1 | 6 | 8102668 | junction | tumor | 0.59 | 17.82 |
| 629 | ENST00000379742.4 | POSTN | 13 | 38172745 | junction | tumor | 0.01 | 11.69 |
| 630 | ENST00000379923.5 | ACO1 | 9 | 32384733 | junction | tumor | 0.81 | 16.28 |
| 631 | ENST00000380071.7 | RFC3 | 13 | 34392402 | junction | tumor | 0.48 | 15.11 |
| 632 | ENST00000381134.7 | ARSE | X | 2882265 | junction | tumor | 0.00 | 24.51 |
| 633 | ENST00000382848.4 | GJB2 | 13 | 20766922 | junction | tumor | 0.09 | 18.88 |
| 634 | ENST00000389614.5 | GPX2 | 14 | 65409223 | junction | tumor | 0.10 | 652.10 |
| 635 | ENST00000391967.6 | LAD1 | 1 | 201368397 | junction | tumor | 0.00 | 16.64 |
| 636 | ENST00000393316.7 | BCL2L15 | 1 | 114429871 | junction | tumor | 0.11 | 11.11 |
| 637 | ENST00000393366.6 | ATP5G1 | 17 | 46970272 | junction | tumor | 0.63 | 40.08 |
| 638 | ENST00000393725.6 | KIAA1191 | 5 | 175788605 | junction | tumor | 0.75 | 11.70 |
| 639 | ENST00000394201.8 | SCOC | 4 | 141294871 | junction | tumor | 0.53 | 14.61 |
| 640 | ENST00000394265.5 | PPP1R1B | 17 | 37784875 | junction | tumor | 0.00 | 81.30 |
| 641 | ENST00000394267.2 | PPP1R1B | 17 | 37784959 | junction | tumor | 0.00 | 87.42 |
| 642 | ENST00000395641.2 | NUPR1 | 16 | 28550117 | junction | tumor | 0.45 | 37.05 |
| 643 | ENST00000397542.6 | CDHR5 | 11 | 624818 | junction | tumor | 0.02 | 18.38 |
| 644 | ENST00000397714.6 | SEPTIN10 | 2 | 110371375 | junction | tumor | 0.14 | 14.96 |
| 645 | ENST00000397995.2 | RNASE4 | 14 | 21152855 | junction | tumor | 0.56 | 10.20 |
| 646 | ENST00000401412.5 | AGR2 | 7 | 16844577 | junction | tumor | 0.00 | 103.38 |
| 647 | ENST00000403444.7 | CEACAM1 | 19 | 43032463 | junction | tumor | 0.00 | 33.67 |
| 648 | ENST00000405271.5 | EPCAM | 2 | 47572362 | junction | tumor | 0.00 | 20.99 |
| 649 | ENST00000410036.2 | MZT2A | 2 | 132250293 | junction | tumor | 0.00 | 12.64 |
| 650 | ENST00000416348.1 | ADIRF | 10 | 88728362 | junction | tumor | 0.00 | 22.43 |
| 651 | ENST00000419304.6 | AGR2 | 7 | 16844559 | junction | tumor | 0.00 | 257.72 |
| 652 | ENST00000419308.6 | FOXA2 | 20 | 22564830 | junction | tumor | 0.00 | 13.51 |
| 653 | ENST00000420892.1 | HTRA1 | 10 | 124266401 | junction | tumor | 0.98 | 24.46 |
| 654 | ENST00000423485.5 | TOP2A | 17 | 38574023 | junction | tumor | 0.44 | 31.06 |
| 655 | ENST00000425042.6 | HID1 | 17 | 72968686 | junction | tumor | 0.23 | 13.99 |
| 656 | ENST00000425340.2 | FUT2 | 19 | 49199346 | junction | tumor | 0.02 | 13.10 |
| 657 | ENST00000428445.1 | VARS | 6 | 31750307 | junction | tumor | 0.78 | 10.22 |
| 658 | ENST00000428849.6 | KIFC1 | 6 | 33359774 | junction | tumor | 0.60 | 17.24 |
| 659 | ENST00000429772.6 | TMEM106C | 12 | 48357487 | junction | tumor | 0.70 | 34.02 |
| 660 | ENST00000430118.1 | HMGB3 | X | 150154222 | junction | tumor | 0.52 | 12.82 |
| 661 | ENST00000433307.2 | PLEKHA1 | 10 | 124152857 | junction | tumor | 0.98 | 13.38 |
| 662 | ENST00000441275.5 | BDH1 | 3 | 197282652 | junction | tumor | 0.87 | 17.80 |
| 663 | ENST00000444124.6 | DDC | 7 | 50632982 | junction | tumor | 0.00 | 21.66 |
| 664 | ENST00000448599.2 | PHGR1 | 15 | 40643263 | junction | tumor | 0.00 | 289.14 |
| 665 | ENST00000450427.1 | PRR15 | 7 | 29605369 | junction | tumor | 0.00 | 10.59 |
| 666 | ENST00000450894.7 | ITGB4 | 17 | 73717692 | junction | tumor | 0.14 | 50.86 |
| 667 | ENST00000452441.5 | DDR1 | 6 | 30852487 | junction | tumor | 0.07 | 51.62 |
| 668 | ENST00000455712.5 | POLR2H | 3 | 184080512 | junction | tumor | 0.94 | 11.92 |
| 669 | ENST00000463201.2 | PRAP1 | 10 | 135164937 | junction | tumor | 0.00 | 54.46 |
| 670 | ENST00000467415.5 | TMEM14C | 6 | 10723474 | junction | tumor | 0.69 | 26.73 |
| 671 | ENST00000467905.5 | AK2 | 1 | 33502337 | junction | tumor | 0.00 | 11.87 |
| 672 | ENST00000469862.1 | CENPF | 1 | 214828746 | junction | tumor | 0.39 | 10.26 |
| 673 | ENST00000472782.1 | ATP5G3 | 2 | 176046384 | junction | tumor | 0.62 | 10.87 |
| 674 | ENST00000476587.1 | NDUFB5 | 3 | 179334832 | junction | tumor | 0.89 | 14.92 |
| 675 | ENST00000478194.1 | FERMT1 | 20 | 6074721 | junction | tumor | 0.00 | 27.00 |
| 676 | ENST00000478869.1 | WDR12 | 2 | 203765748 | junction | tumor | 0.71 | 14.50 |
| 677 | ENST00000479419.1 | IFT172 | 2 | 27668796 | junction | tumor | 0.00 | 37.10 |
| 678 | ENST00000484713.1 | LAMB2 | 3 | 49158866 | junction | tumor | 0.41 | 13.87 |
| 679 | ENST00000489477.1 | NDUFAF4 | 6 | 97345542 | junction | tumor | 0.66 | 10.30 |
| 680 | ENST00000490807.5 | NTPCR | 1 | 233086490 | junction | tumor | 0.77 | 10.54 |
| 681 | ENST00000494446.1 | FN1 | 2 | 216230228 | junction | tumor | 0.79 | 52.10 |
| 682 | ENST00000494801.5 | TCEAL4 | X | 102840552 | junction | tumor | 0.60 | 13.59 |
| 683 | ENST00000495558.1 | VWA1 | 1 | 1370526 | junction | tumor | 0.00 | 13.92 |
| 684 | ENST00000496195.1 | GLRX3 | 10 | 131977684 | junction | tumor | 0.98 | 10.30 |
| 685 | ENST00000497734.5 | SRC | 20 | 35973290 | junction | tumor | 0.00 | 12.44 |
| 686 | ENST00000507614.1 | TIMM8B | 11 | 111957364 | junction | tumor | 0.80 | 10.54 |
| 687 | ENST00000507699.1 | PALLD | 4 | 169819865 | junction | tumor | 0.31 | 14.56 |
| 688 | ENST00000514985.5 | SEPP1 | 5 | 42811938 | junction | tumor | 0.59 | 49.95 |
| 689 | ENST00000520271.5 | COX6C | 8 | 100905671 | junction | tumor | 0.51 | 12.86 |
| 690 | ENST00000523677.5 | C1orf210 | 1 | 43751115 | junction | tumor | 0.00 | 13.49 |
| 691 | ENST00000524832.5 | CHID1 | 11 | 902198 | junction | tumor | 0.42 | 10.82 |
| 692 | ENST00000525657.1 | C1QTNF5 | 11 | 119211405 | junction | tumor | 0.05 | 17.27 |
| 693 | ENST00000526202.5 | LMO7 | 13 | 76334967 | junction | tumor | 0.09 | 23.11 |
| 694 | ENST00000527106.5 | FUT6 | 19 | 5838688 | junction | tumor | 0.00 | 13.22 |
| 695 | ENST00000528430.2 | PPP1R16A | 8 | 145726677 | junction | tumor | 0.92 | 22.33 |
| 696 | ENST00000530094.5 | CTNND1 | 11 | 57529518 | junction | tumor | 0.00 | 26.61 |
| 697 | ENST00000533827.5 | VPS51 | 11 | 64877063 | junction | tumor | 0.00 | 12.01 |
| 698 | ENST00000534378.5 | ILVBL | 19 | 15236467 | junction | tumor | 0.64 | 11.41 |
| 699 | ENST00000537496.5 | MMAB | 12 | 110011152 | junction | tumor | 0.93 | 12.43 |
| 700 | ENST00000541754.1 | NNMT | 11 | 114168120 | junction | tumor | 0.72 | 23.55 |
| 701 | ENST00000542056.1 | GPRC5A | 12 | 13044598 | junction | tumor | 0.00 | 23.91 |
| 702 | ENST00000543445.5 | LDHA | 11 | 18416188 | junction | tumor | 0.00 | 71.47 |
| 703 | ENST00000543623.5 | PLCD3 | 17 | 43192462 | junction | tumor | 0.27 | 14.58 |
| 704 | ENST00000546314.5 | STARD10 | 11 | 72493311 | junction | tumor | 0.00 | 31.66 |
| 705 | ENST00000546485.5 | RPL41 | 12 | 56510443 | junction | tumor | 0.75 | 48.15 |
| 706 | ENST00000547281.5 | CDK4 | 12 | 58145958 | junction | tumor | 0.83 | 30.31 |
| 707 | ENST00000547838.2 | FAM109A | 12 | 111801492 | junction | tumor | 0.94 | 11.49 |
| 708 | ENST00000548169.2 | ATP2A2 | 12 | 110729929 | junction | tumor | 0.34 | 13.63 |
| 709 | ENST00000552128.2 | TSPAN8 | 12 | 71532931 | junction | tumor | 0.00 | 15.81 |
| 710 | ENST00000552561.5 | TMEM106C | 12 | 48357416 | junction | tumor | 0.38 | 17.25 |
| 711 | ENST00000554989.1 | CKB | 14 | 103987600 | junction | tumor | 0.75 | 83.82 |
| 712 | ENST00000557049.1 | GPX2 | 14 | 65409385 | junction | tumor | 0.00 | 18.28 |
| 713 | ENST00000558580.1 | SORD | 15 | 45328542 | junction | tumor | 0.95 | 15.73 |
| 714 | ENST00000559087.5 | BMP4 | 14 | 54423477 | junction | tumor | 0.00 | 13.08 |
| 715 | ENST00000562684.5 | HN1L | 16 | 1728357 | junction | tumor | 0.25 | 19.21 |
| 716 | ENST00000564043.1 | NQO1 | 16 | 69760404 | junction | tumor | 0.32 | 89.24 |
| 717 | ENST00000581920.1 | TYMS | 18 | 667752 | junction | tumor | 0.88 | 11.79 |
| 718 | ENST00000583327.2 | ITGB4 | 17 | 73747929 | junction | tumor | 0.12 | 32.84 |
| 719 | ENST00000586005.5 | SMIM22 | 16 | 4845450 | junction | tumor | 0.00 | 103.03 |
| 720 | ENST00000587251.5 | LGALS3BP | 17 | 76975906 | junction | tumor | 0.00 | 48.36 |
| 721 | ENST00000588605.5 | C19orf33 | 19 | 38794923 | junction | tumor | 0.22 | 18.46 |
| 722 | ENST00000589378.5 | TJP3 | 19 | 3721908 | junction | tumor | 0.13 | 11.74 |
| 723 | ENST00000591795.1 | DENND1C | 19 | 6469607 | junction | tumor | 0.56 | 11.26 |
| 724 | ENST00000594443.5 | FBL | 19 | 40336931 | junction | tumor | 0.00 | 19.57 |
| 725 | ENST00000594605.5 | STAP2 | 19 | 4338649 | junction | tumor | 0.06 | 27.33 |
| 726 | ENST00000595110.1 | FAM83E | 19 | 49117965 | junction | tumor | 0.00 | 13.13 |
| 727 | ENST00000597153.5 | LGALS4 | 19 | 39303482 | junction | tumor | 0.00 | 20.51 |
| 728 | ENST00000601623.5 | LSR | 19 | 35740271 | junction | tumor | 0.21 | 24.39 |
| 729 | ENST00000610552.4 | SLC2A8 | 9 | 130159565 | junction | tumor | 0.65 | 11.38 |
| 730 | ENST00000612794.1 | GPX2 | 14 | 65409328 | junction | tumor | 0.00 | 23.73 |
| 731 | ENST00000612809.4 | C8orf59 | 8 | 86132535 | junction | tumor | 0.94 | 54.53 |
| 732 | ENST00000616154.1 | CDX1 | 5 | 149546475 | junction | tumor | 0.00 | 138.07 |
| 733 | ENST00000616727.4 | MUC13 | 3 | 124653505 | junction | tumor | 0.01 | 221.05 |
| 734 | ENST00000618855.4 | TMPRSS4 | 11 | 117948020 | junction | tumor | 0.00 | 10.45 |
| 735 | ENST00000619895.4 | TMC4 | 19 | 54676734 | junction | tumor | 0.35 | 33.05 |
| 736 | ENST00000620753.4 | EI24 | 11 | 125439410 | junction | tumor | 0.63 | 12.17 |
| 737 | ENST00000008938.4 | PGLYRP1 | 19 | 46525993 | junction | blood | 148.76 | 0.05 |
| 738 | ENST00000199708.2 | HBQ1 | 16 | 230580 | junction | blood | 45.79 | 0.00 |
| 739 | ENST00000216338.8 | GZMH | 14 | 25078765 | junction | blood | 15.58 | 0.99 |
| 740 | ENST00000221515.5 | RETN | 19 | 7734007 | junction | blood | 72.49 | 0.19 |
| 741 | ENST00000221954.6 | CEACAM4 | 19 | 42133268 | junction | blood | 32.81 | 0.13 |
| 742 | ENST00000225538.3 | P2RX1 | 17 | 3819383 | junction | blood | 15.55 | 0.30 |
| 743 | ENST00000232014.8 | BCL6 | 3 | 187453878 | junction | blood | 19.07 | 0.18 |
| 744 | ENST00000234347.9 | PRTN3 | 19 | 841069 | junction | blood | 13.78 | 0.00 |
| 745 | ENST00000236826.7 | MMP8 | 11 | 102595485 | junction | blood | 20.68 | 0.04 |
| 746 | ENST00000244709.8 | TREM1 | 6 | 41254345 | junction | blood | 67.12 | 0.63 |
| 747 | ENST00000246115.4 | S1PR4 | 19 | 3180330 | junction | blood | 127.44 | 0.94 |
| 748 | ENST00000246549.2 | FFAR2 | 19 | 35942667 | junction | blood | 58.85 | 0.34 |
| 749 | ENST00000246657.2 | CCR7 | 17 | 38721652 | junction | blood | 11.89 | 0.75 |
| 750 | ENST00000259396.8 | ORM1 | 9 | 117085527 | junction | blood | 34.89 | 0.14 |
| 751 | ENST00000262407.5 | ITGA2B | 17 | 42466654 | junction | blood | 12.34 | 0.04 |
| 752 | ENST00000264972.9 | ZAP70 | 2 | 98330137 | junction | blood | 15.69 | 0.72 |
| 753 | ENST00000279452.10 | CD44 | 11 | 35198287 | junction | blood | 10.76 | 0.00 |
| 754 | ENST00000281703.10 | GLT1D1 | 12 | 129338194 | junction | blood | 22.75 | 0.02 |
| 755 | ENST00000290075.10 | SLC25A37 | 8 | 23386725 | junction | blood | 45.08 | 0.87 |
| 756 | ENST00000292432.9 | HK3 | 5 | 176326268 | junction | blood | 98.07 | 0.60 |
| 757 | ENST00000295683.2 | CXCR1 | 2 | 219031631 | junction | blood | 355.57 | 0.18 |
| 758 | ENST00000296435.2 | CAMP | 3 | 48265202 | junction | blood | 27.17 | 0.06 |
| 759 | ENST00000296487.8 | PPM1M | 3 | 52280330 | junction | blood | 13.09 | 0.41 |
| 760 | ENST00000297239.10 | SYTL3 | 6 | 159082417 | junction | blood | 10.79 | 0.54 |
| 761 | ENST00000299663.7 | CLEC4E | 12 | 8693357 | junction | blood | 29.27 | 0.20 |
| 762 | ENST00000302017.3 | ZNF467 | 7 | 149470197 | junction | blood | 25.10 | 0.54 |
| 763 | ENST00000303531.11 | PRKCB | 16 | 23847669 | junction | blood | 16.08 | 0.36 |
| 764 | ENST00000303757.12 | LST1 | 6 | 31555095 | junction | blood | 11.09 | 0.23 |
| 765 | ENST00000307564.8 | AKNA | 9 | 117156637 | junction | blood | 11.95 | 0.38 |
| 766 | ENST00000310544.8 | PHOSPHO1 | 17 | 47307830 | junction | blood | 30.92 | 0.08 |
| 767 | ENST00000318507.6 | CXCR2 | 2 | 218991076 | junction | blood | 157.27 | 0.28 |
| 768 | ENST00000326165.10 | CD300LF | 17 | 72708963 | junction | blood | 18.99 | 0.70 |
| 769 | ENST00000328118.7 | FMNL1 | 17 | 43299554 | junction | blood | 11.80 | 0.41 |
| 770 | ENST00000329021.9 | NFAM1 | 22 | 42828243 | junction | blood | 43.63 | 0.94 |
| 771 | ENST00000329410.3 | C16orf54 | 16 | 29757232 | junction | blood | 24.31 | 0.49 |
| 772 | ENST00000332549.7 | IL1R2 | 2 | 102608473 | junction | blood | 112.29 | 0.55 |
| 773 | ENST00000336577.8 | MMP25 | 16 | 3097017 | junction | blood | 194.37 | 0.54 |
| 774 | ENST00000336906.4 | HBG2 | 11 | 5275867 | junction | blood | 104.85 | 0.00 |
| 775 | ENST00000338372.6 | VSTM1 | 19 | 54566998 | junction | blood | 10.71 | 0.00 |
| 776 | ENST00000342571.7 | MKNK1 | 1 | 47051546 | junction | blood | 14.42 | 0.02 |
| 777 | ENST00000343534.9 | C1orf162 | 1 | 112016652 | junction | blood | 37.68 | 0.74 |
| 778 | ENST00000346667.8 | IKZF1 | 7 | 50344518 | junction | blood | 11.73 | 0.24 |
| 779 | ENST00000352818.8 | CD44 | 11 | 35160917 | junction | blood | 14.11 | 0.00 |
| 780 | ENST00000354352.9 | SLC11A1 | 2 | 219247098 | junction | blood | 111.80 | 0.63 |
| 781 | ENST00000355524.7 | FCAR | 19 | 55385779 | junction | blood | 53.04 | 0.07 |
| 782 | ENST00000356815.3 | HBM | 16 | 216088 | junction | blood | 163.82 | 0.00 |
| 783 | ENST00000356838.7 | TMEM71 | 8 | 133772722 | junction | blood | 16.78 | 0.36 |
| 784 | ENST00000356864.3 | TNFRSF10C | 8 | 22960694 | junction | blood | 108.86 | 0.56 |
| 785 | ENST00000357198.8 | DOK3 | 5 | 176936803 | junction | blood | 66.68 | 0.74 |
| 786 | ENST00000357260.5 | FAM212B | 1 | 112281808 | junction | blood | 12.23 | 0.67 |
| 787 | ENST00000358375.8 | OSCAR | 19 | 54604047 | junction | blood | 18.93 | 0.28 |
| 788 | ENST00000367025.7 | TRAF3IP3 | 1 | 209929654 | junction | blood | 17.52 | 0.64 |
| 789 | ENST00000367053.5 | CR1 | 1 | 207669733 | junction | blood | 10.47 | 0.02 |
| 790 | ENST00000367535.7 | NCF2 | 1 | 183559291 | junction | blood | 75.23 | 0.98 |
| 791 | ENST00000367568.4 | STX11 | 6 | 144471840 | junction | blood | 24.42 | 0.79 |
| 792 | ENST00000367972.8 | FCGR2A | 1 | 161475342 | junction | blood | 27.69 | 0.40 |
| 793 | ENST00000368015.1 | ARHGAP30 | 1 | 161039410 | junction | blood | 20.61 | 0.58 |
| 794 | ENST00000368732.5 | S100A8 | 1 | 153363333 | junction | blood | 53.91 | 0.00 |
| 795 | ENST00000368737.4 | S100A12 | 1 | 153348028 | junction | blood | 1099.02 | 0.31 |
| 796 | ENST00000371806.3 | FCN1 | 9 | 137809615 | junction | blood | 379.64 | 0.88 |
| 797 | ENST00000373103.5 | CSF3R | 1 | 36948412 | junction | blood | 61.20 | 0.04 |
| 798 | ENST00000373925.5 | THEMIS2 | 1 | 28199176 | junction | blood | 12.15 | 0.53 |
| 799 | ENST00000374005.7 | FGR | 1 | 27961576 | junction | blood | 32.42 | 0.29 |
| 800 | ENST00000374163.5 | RPS6KA1 | 1 | 26872526 | junction | blood | 17.41 | 0.87 |
| 801 | ENST00000375448.4 | PADI4 | 1 | 17634809 | junction | blood | 95.04 | 0.03 |
| 802 | ENST00000375862.6 | HCK | 20 | 30640289 | junction | blood | 20.11 | 0.09 |
| 803 | ENST00000376670.7 | GATA1 | X | 48645053 | junction | blood | 11.17 | 0.01 |
| 804 | ENST00000377497.7 | RASGRP2 | 11 | 64512214 | junction | blood | 48.02 | 0.55 |
| 805 | ENST00000378023.8 | FAM65B | 6 | 24877179 | junction | blood | 15.67 | 0.00 |
| 806 | ENST00000379400.7 | RASSF2 | 20 | 4804203 | junction | blood | 13.75 | 0.82 |
| 807 | ENST00000380299.3 | HBD | 11 | 5255572 | junction | blood | 138.84 | 0.07 |
| 808 | ENST00000381112.7 | CCM2 | 7 | 45067396 | junction | blood | 17.78 | 0.00 |
| 809 | ENST00000381153.7 | C11orf21 | 11 | 2322986 | junction | blood | 11.33 | 0.09 |
| 810 | ENST00000381603.7 | SIRPB1 | 20 | 1600515 | junction | blood | 26.01 | 0.00 |
| 811 | ENST00000391750.5 | LILRB3 | 19 | 54727293 | junction | blood | 65.61 | 0.41 |
| 812 | ENST00000393450.5 | MYL4 | 17 | 45286923 | junction | blood | 22.77 | 0.00 |
| 813 | ENST00000393847.5 | DPEP2 | 16 | 68033278 | junction | blood | 24.04 | 0.44 |
| 814 | ENST00000394991.7 | SNCA | 4 | 90758113 | junction | blood | 32.19 | 0.35 |
| 815 | ENST00000397147.6 | NCF4 | 22 | 37257245 | junction | blood | 154.32 | 0.92 |
| 816 | ENST00000398421.6 | NCF1 | 7 | 74188450 | junction | blood | 11.04 | 0.04 |
| 817 | ENST00000399173.5 | FGR | 1 | 27952557 | junction | blood | 101.05 | 0.26 |
| 818 | ENST00000399753.2 | MSRB1 | 16 | 1993103 | junction | blood | 11.22 | 0.15 |
| 819 | ENST00000409220.5 | ARHGAP25 | 2 | 69002552 | junction | blood | 23.89 | 0.31 |
| 820 | ENST00000409930.3 | IL1RN | 2 | 113885317 | junction | blood | 21.18 | 0.85 |
| 821 | ENST00000416215.6 | PTPN6 | 12 | 7060683 | junction | blood | 12.72 | 0.88 |
| 822 | ENST00000422400.6 | VNN2 | 6 | 133078810 | junction | blood | 14.59 | 0.10 |
| 823 | ENST00000423345.4 | PRAM1 | 19 | 8567449 | junction | blood | 25.32 | 0.30 |
| 824 | ENST00000433194.6 | CDK5RAP2 | 9 | 123165594 | junction | blood | 22.55 | 0.52 |
| 825 | ENST00000441002.1 | IL1R2 | 2 | 102625104 | junction | blood | 16.30 | 0.00 |
| 826 | ENST00000445347.1 | LILRB3 | 19 | 54726815 | junction | blood | 49.26 | 0.10 |
| 827 | ENST00000445961.5 | RPS9 | 19 | 54704829 | junction | blood | 14.72 | 0.89 |
| 828 | ENST00000447110.5 | PIK3R5 | 17 | 8868913 | junction | blood | 12.89 | 0.29 |
| 829 | ENST00000449131.6 | BEST1 | 11 | 61717899 | junction | blood | 13.35 | 0.54 |
| 830 | ENST00000454703.6 | ACSL1 | 4 | 185747070 | junction | blood | 35.07 | 0.78 |
| 831 | ENST00000460442.5 | ZDHHC19 | 3 | 195925660 | junction | blood | 22.43 | 0.04 |
| 832 | ENST00000464431.1 | ABTB1 | 3 | 127395274 | junction | blood | 20.22 | 0.70 |
| 833 | ENST00000464465.6 | CSF3R | 1 | 36937034 | junction | blood | 259.11 | 0.50 |
| 834 | ENST00000465814.5 | ARAP1 | 11 | 72432895 | junction | blood | 10.09 | 0.58 |
| 835 | ENST00000467786.1 | FGD3 | 9 | 95737687 | junction | blood | 43.15 | 0.29 |
| 836 | ENST00000474085.5 | IL1R2 | 2 | 102638711 | junction | blood | 24.35 | 0.04 |
| 837 | ENST00000475226.1 | HBB | 11 | 5247807 | junction | blood | 2709.48 | 0.00 |
| 838 | ENST00000477801.1 | S100A8 | 1 | 153362871 | junction | blood | 161.47 | 0.07 |
| 839 | ENST00000480395.5 | TRIM22 | 11 | 5717885 | junction | blood | 17.36 | 0.70 |
| 840 | ENST00000481568.2 | C10orf54 | 10 | 73521355 | junction | blood | 31.27 | 0.90 |
| 841 | ENST00000483750.5 | WAS | X | 48542374 | junction | blood | 34.49 | 0.17 |
| 842 | ENST00000485743.1 | HBB | 11 | 5248160 | junction | blood | 128.52 | 0.00 |
| 843 | ENST00000485928.5 | PHC2 | 1 | 33815198 | junction | blood | 11.05 | 0.73 |
| 844 | ENST00000486097.1 | NCF1 | 7 | 74197975 | junction | blood | 12.51 | 0.06 |
| 845 | ENST00000487540.6 | CSF3R | 1 | 36937839 | junction | blood | 47.30 | 0.17 |
| 846 | ENST00000488171.5 | CD55 | 1 | 207495210 | junction | blood | 25.43 | 0.43 |
| 847 | ENST00000488945.5 | GNLY | 2 | 85912344 | junction | blood | 11.68 | 0.09 |
| 848 | ENST00000489358.5 | NAMPT | 7 | 105925274 | junction | blood | 30.07 | 0.00 |
| 849 | ENST00000491955.5 | MPP1 | X | 154009875 | junction | blood | 10.84 | 0.42 |
| 850 | ENST00000493242.1 | LILRB2 | 19 | 54784816 | junction | blood | 22.65 | 0.13 |
| 851 | ENST00000494185.1 | LMNB1 | 5 | 126147590 | junction | blood | 10.53 | 0.00 |
| 852 | ENST00000496823.1 | BCL6 | 3 | 187463198 | junction | blood | 21.33 | 0.20 |
| 853 | ENST00000497259.5 | ARHGAP25 | 2 | 69034612 | junction | blood | 25.49 | 0.72 |
| 854 | ENST00000497920.1 | ARID5A | 2 | 97213254 | junction | blood | 11.90 | 0.30 |
| 855 | ENST00000509314.5 | FBXL5 | 4 | 15661350 | junction | blood | 10.24 | 0.00 |
| 856 | ENST00000509339.1 | MXD3 | 5 | 176734782 | junction | blood | 30.07 | 0.08 |
| 857 | ENST00000510784.6 | FAM65B | 6 | 25042079 | junction | blood | 61.47 | 0.19 |
| 858 | ENST00000513001.5 | ACSL1 | 4 | 185678973 | junction | blood | 17.65 | 0.10 |
| 859 | ENST00000520174.5 | DMTN | 8 | 21914647 | junction | blood | 16.02 | 0.00 |
| 860 | ENST00000523022.5 | CA1 | 8 | 86290275 | junction | blood | 40.92 | 0.11 |
| 861 | ENST00000525270.5 | VNN2 | 6 | 133084522 | junction | blood | 38.37 | 0.12 |
| 862 | ENST00000527146.1 | IFITM2 | 11 | 308438 | junction | blood | 18.56 | 0.63 |
| 863 | ENST00000528780.5 | IFITM1 | 11 | 313691 | junction | blood | 47.88 | 0.31 |
| 864 | ENST00000529635.5 | TBC1D10C | 11 | 67171825 | junction | blood | 16.89 | 0.34 |
| 865 | ENST00000533968.1 | SPI1 | 11 | 47399860 | junction | blood | 12.90 | 0.22 |
| 866 | ENST00000535669.6 | CD37 | 19 | 49838866 | junction | blood | 33.70 | 0.19 |
| 867 | ENST00000540998.5 | CDC42SE1 | 1 | 151031955 | junction | blood | 10.52 | 0.00 |
| 868 | ENST00000542481.1 | ATG16L2 | 11 | 72535167 | junction | blood | 25.18 | 0.37 |
| 869 | ENST00000543576.5 | DENND1C | 19 | 6481690 | junction | blood | 12.06 | 0.00 |
| 870 | ENST00000544232.5 | TMEM91 | 19 | 41882759 | junction | blood | 12.57 | 0.20 |
| 871 | ENST00000544665.7 | ITGAM | 16 | 31271413 | junction | blood | 56.38 | 0.99 |
| 872 | ENST00000546704.1 | ARHGAP9 | 12 | 57868658 | junction | blood | 19.20 | 0.13 |
| 873 | ENST00000552370.5 | TMBIM6 | 12 | 50144488 | junction | blood | 26.69 | 0.00 |
| 874 | ENST00000553070.5 | NFE2 | 12 | 54694585 | junction | blood | 23.61 | 0.00 |
| 875 | ENST00000558012.5 | PSTPIP1 | 15 | 77287950 | junction | blood | 26.47 | 0.31 |
| 876 | ENST00000564662.1 | COTL1 | 16 | 84651107 | junction | blood | 10.13 | 0.81 |
| 877 | ENST00000564905.1 | XPO6 | 16 | 28145162 | junction | blood | 12.98 | 0.47 |
| 878 | ENST00000568763.1 | CORO1A | 16 | 30195046 | junction | blood | 45.17 | 0.63 |
| 879 | ENST00000570106.6 | SIGLEC5 | 19 | 52133552 | junction | blood | 40.53 | 0.18 |
| 880 | ENST00000572782.1 | ARRB2 | 17 | 4618338 | junction | blood | 16.63 | 0.33 |
| 881 | ENST00000576628.1 | ACAP1 | 17 | 7240106 | junction | blood | 20.20 | 0.18 |
| 882 | ENST00000577894.1 | EVI2B | 17 | 29640997 | junction | blood | 14.34 | 0.00 |
| 883 | ENST00000578067.5 | LIMD2 | 17 | 61776617 | junction | blood | 10.86 | 0.22 |
| 884 | ENST00000578402.5 | LIMD2 | 17 | 61777729 | junction | blood | 31.49 | 0.77 |
| 885 | ENST00000585901.6 | TYROBP | 19 | 36399070 | junction | blood | 13.40 | 0.19 |
| 886 | ENST00000586946.1 | TYROBP | 19 | 36399077 | junction | blood | 23.56 | 0.61 |
| 887 | ENST00000587259.5 | VMP1 | 17 | 57807378 | junction | blood | 10.58 | 0.05 |
| 888 | ENST00000587856.1 | FMNL1 | 17 | 43311567 | junction | blood | 22.20 | 0.28 |
| 889 | ENST00000588673.3 | OAZ1 | 19 | 2270675 | junction | blood | 24.28 | 0.69 |
| 890 | ENST00000589900.5 | ICAM3 | 19 | 10450215 | junction | blood | 21.24 | 0.00 |
| 891 | ENST00000592860.2 | CFD | 19 | 859765 | junction | blood | 16.53 | 0.85 |
| 892 | ENST00000595042.5 | FPR1 | 19 | 52255067 | junction | blood | 63.93 | 0.18 |
| 893 | ENST00000595217.1 | NKG7 | 19 | 51875633 | junction | blood | 33.26 | 0.52 |
| 894 | ENST00000595325.5 | MYO1F | 19 | 8642191 | junction | blood | 13.66 | 0.09 |
| 895 | ENST00000595636.1 | GMFG | 19 | 39826614 | junction | blood | 10.44 | 0.68 |
| 896 | ENST00000595840.1 | LRRC25 | 19 | 18508263 | junction | blood | 36.84 | 0.88 |
| 897 | ENST00000596764.5 | VAV1 | 19 | 6773022 | junction | blood | 12.25 | 0.59 |
| 898 | ENST00000597852.5 | CD37 | 19 | 49838832 | junction | blood | 13.98 | 0.20 |
| 899 | ENST00000599180.2 | FFAR2 | 19 | 35939281 | junction | blood | 24.79 | 0.00 |
| 900 | ENST00000599716.5 | SHKBP1 | 19 | 41082891 | junction | blood | 10.66 | 0.22 |
| 901 | ENST00000600626.1 | C5AR2 | 19 | 47840426 | junction | blood | 12.99 | 0.11 |
| 902 | ENST00000600972.1 | JUND | 19 | 18391266 | junction | blood | 17.49 | 0.60 |
| 903 | ENST00000605039.5 | BIN2 | 12 | 51717806 | junction | blood | 11.02 | 0.11 |
| 904 | ENST00000611028.2 | FOLR3 | 11 | 71846814 | junction | blood | 11.13 | 0.00 |
| 905 | ENST00000615439.4 | RASGRP4 | 19 | 38916709 | junction | blood | 10.11 | 0.00 |
| 906 | ENST00000615825.1 | MME | 3 | 154802116 | junction | blood | 12.37 | 0.88 |
| 907 | ENST00000618265.4 | CD177 | 19 | 43857918 | junction | blood | 222.54 | 0.73 |
| 908 | ENST00000620541.4 | RIN3 | 14 | 92980320 | junction | blood | 17.39 | 0.84 |

Columns
ID: feature index
Transcript: transcript ID
Gene: gene name
Chr: chromosome
Site: coordinate of nucleosome-depleted site (GRCh37)
Region: location of nucleosome-depleted site
Group: gene group based on its expression in blood and tumor
FPKM_{blood}: FPKM value in normal blood
FPKMₜᵤₘₒᵣ: FPKM value in tumor of colorectal cancer

TABLE S4
Coefficients for the selected NDRs in the trained models
(CRC model and CRC + BRCA model).
CRC model

| Feature | Gene | Transcript | Region | Group | Coefficient |
|---|---|---|---|---|---|
| 1 | SHKBP1 | ENST00000599716 | junction | blood | 0.607 |
| 2 | ACSL1 | ENST00000454703 | junction | blood | 0.431 |
| 3 | BCAR1 | ENST00000162330 | junction | tumor | −0.321 |
| 4 | RAB25 | ENST00000361084 | promoter | tumor | −0.213 |
| 5 | PRTN3 | ENST00000234347 | promoter | blood | 0.062 |
| 6 | LSR | ENST00000605618 | promoter | tumor | −0.174 |

Columns
Gene: gene name
Transcript: transcript ID
Region: location of nucleosome-depleted site
Group: gene group based on its expression in blood and tumor
Coefficient: value of the regression coefficient. The intercept value is 0.4368.
CRC + BRCA model

| Feature | Gene | Transcript | Region | Group | Coefficient |
|---|---|---|---|---|---|
| 1 | SLC11A1 | ENST00000465984 | promoter | blood | 0.150 |
| 2 | NLRP12 | ENST00000324134 | promoter | blood | 0.181 |
| 3 | PRTN3 | ENST00000234347 | promoter | blood | 0.124 |
| 4 | HMBS | ENST00000392841 | promoter | blood | 0.251 |
| 5 | LILRB3 | ENST00000460208 | promoter | blood | 0.140 |
| 6 | ACSL1 | ENST00000513001 | junction | blood | 0.106 |
| 7 | GP9 | ENST00000307395 | junction | blood | 0.251 |
| 8 | MX2 | ENST00000398632 | promoter | blood | 0.106 |
| 9 | RASGRP4 | ENST00000615340 | promoter | blood | 0.222 |
| 10 | ATG16L2 | ENST00000542481 | promoter | blood | 0.166 |

Columns
Gene: gene name
Transcript: transcript ID
Region: location of nucleosome-depleted site
Group: gene group based on its expression in blood and tumor
Coefficient: value of the regression coefficient. The intercept value is −1.3719.

TABLE S4
Columns
Gene: gene name
Transcript: transcript ID
Region: location of nucleosome-depleted site
Group: gene group based on its expression in blood and tumor
Coefficient: value of the regression coefficient. The intercept value is 0.4368.
Coefficients for the selected NDRs in the trained models (CRC model and
CRC + BRCA model).

| CRC model | | | | | |
|---|---|---|---|---|---|
| Feature | Gene | T ranscript | Region | Group | Coefficient |
| 1 | SHKBP1 | ENST00000599716 | junction | blood | 0.607 |
| 2 | ACSL1 | ENST00000454703 | junction | blood | 0.431 |
| 3 | BCAR1 | ENST00000162330 | junction | tumor | -0.321 |
| 4 | RAB25 | ENST00000361084 | promoter | tumor | -0.213 |
| 5 | PRTN3 | ENST00000234347 | promoter | blood | 0.062 |
| 6 | LSR | ENST00000605618 | promoter | tumor | -0.174 |
| Columns | | | | | |
| Gene: gene name | | | | | |
| Transcript: transcript ID | | | | | |
| Region: location of nucleosome-depleted site | | | | | |
| Group: gene group based on its expression in blood and tumor | | | | | |
| Coefficient: value of the regression coefficient. The intercept value is -1.3719. | | | | | |
| CRC+BRCA model | | | | | |
| Feature | Gene | Transcript | Region | Group | Coefficient |
| 1 | SLC11A1 | ENST00000465984 | promoter | blood | 0.150 |
| 2 | NLRP12 | ENST00000324134 | promoter | blood | 0.181 |
| 3 | PRTN3 | ENST00000234347 | promoter | blood | 0.124 |
| 4 | HMBS | ENST00000392841 | promoter | blood | 0.251 |
| 5 | LILRB3 | ENST00000460208 | promoter | blood | 0.140 |
| 6 | ACSL1 | ENST00000513001 | junction | blood | 0.106 |
| 7 | GP9 | ENST00000307395 | junction | blood | 0.251 |
| 8 | MX2 | ENST00000398632 | promoter | blood | 0.106 |
| 9 | RASGRP4 | ENST00000615340 | promoter | blood | 0.222 |
| 10 | ATG16L2 | ENST00000542481 | promoter | blood | 0.166 |

| Table S5 Observed ctDNA fractions in the LOD analysis for the CRC model. | | | |
|---|---|---|---|
| Sample | Group | Expected ctDNA fraction | Observed ctDNA fraction |
| 1279 221015-0.005 | positive | 0.0050 | 0.0015 |
| 1279_221015-0.010 | positive | 0.0100 | 0.0192 |
| 1279_221015-0.015 | positive | 0.0150 | 0.0170 |
| 1279_221015-0.020 | positive | 0.0200 | 0.0246 |
| 1279_221015-0.025 | positive | 0.0250 | 0.0250 |
| 1279_221015-0.030 | positive | 0.0300 | 0.0389 |
| 1279_221015-0.035 | positive | 0.0350 | 0.0426 |
| 1279_221015-0.040 | positive | 0.0400 | 0.0471 |

| | | | | |
|---|---|---|---|---|
| 1279 | .221015-0.045 | positive | 0.0450 | 0.0750 |
| 1279 | .221015-0.050 | positive | 0.0500 | 0.0692 |
| 1279 | .221015-0.075 | positive | 0.0750 | 0.0964 |
| 1279 | .221015-0.100 | positive | 0.1000 | 0.1093 |
| 1279 | .221015-0.125 | positive | 0.1250 | 0.1210 |
| 1279 | .221015-0.150 | positive | 0.1500 | 0.1381 |
| 1279 | .221015-0.175 | positive | 0.1750 | 0.1250 |
| 1279 | .221015-0.200 | positive | 0.2000 | 0.1452 |
| 1279. | .221015-0.225 | positive | 0.2250 | 0.1590 |
| 1279. | .221015-0.250 | positive | 0.2500 | 0.2252 |
| 1279. | .221015-0.275 | positive | 0.2750 | 0.2856 |
| 1279. | .221015-0.300 | positive | 0.3000 | 0.3152 |
| 1279. | .221015-0.325 | positive | 0.3250 | 0.3456 |
| 1279. | .221015-0.350 | positive | 0.3500 | 0.3587 |
| 1279. | .221015-0.375 | positive | 0.3750 | 0.3994 |
| 1279. | .221015-0.400 | positive | 0.4000 | 0.4318 |
| 1279. | .221015-0.425 | positive | 0.4250 | 0.4363 |
| 1279. | .221015-0.450 | positive | 0.4500 | 0.4634 |
| 1279. | .221015-0.475 | positive | 0.4750 | 0.4652 |
| 1279. | .221015-0.500 | positive | 0.5000 | 0.4776 |
| 1279. | .241116-0.005 | positive | 0.0050 | 0.0033 |
| 1279. | .241116-0.010 | positive | 0.0100 | 0.0150 |
| 1279. | .241116-0.015 | positive | 0.0150 | 0.0420 |
| 1279. | .241116-0.020 | positive | 0.0200 | 0.0368 |
| 1279. | .241116-0.025 | positive | 0.0250 | 0.0396 |
| 1279. | .241116-0.030 | positive | 0.0300 | 0.0439 |
| 1279. | .241116-0.035 | positive | 0.0350 | 0.0439 |
| 1279. | .241116-0.040 | positive | 0.0400 | 0.0576 |
| 1279. | .241116-0.045 | positive | 0.0450 | 0.0574 |
| 1279. | .241116-0.050 | positive | 0.0500 | 0.0692 |
| 1279. | .241116-0.075 | positive | 0.0750 | 0.0508 |
| 1279. | .241116-0.100 | positive | 0.1000 | 0.0707 |
| 1279. | .241116-0.125 | positive | 0.1250 | 0.1357 |
| 1279. | .241116-0.150 | positive | 0.1500 | 0.1457 |
| 1279. | .241116-0.175 | positive | 0.1750 | 0.1366 |
| 1279. | .241116-0.200 | positive | 0.2000 | 0.1632 |
| 1279. | .241116-0.225 | positive | 0.2250 | 0.2000 |
| 1279. | .241116-0.250 | positive | 0.2500 | 0.2310 |
| 1279. | .241116 0.275 | positive | 0.2750 | 0.2914 |
| 1279. | .241116-0.300 | positive | 0.3000 | 0.3281 |
| 1279. | .241116-0.325 | positive | 0.3250 | 0.3622 |
| 1279. | .241116-0.350 | positive | 0.3500 | 0.3690 |
| 1279. | .241116-0.375 | positive | 0.3750 | 0.3762 |
| 1279. | .241116-0.400 | positive | 0.4000 | 0.4185 |
| 1279. | .241116-0.425 | positive | 0.4250 | 0.4311 |
| 1279. | .241116-0.450 | positive | 0.4500 | 0.4618 |
| 1279. | .241116-0.475 | positive | 0.4750 | 0.4958 |
| 1279. | .241116-0.490 | positive | 0.4900 | 0.4937 |
| 512_ | 051015-0.005 | positive | 0.0050 | 0.0049 |

| | | | |
|---|---|---|---|
| 512_051015-0.010 | positive | 0.0100 | 0.0077 |
| 512 051015-0.015 | positive | 0.0150 | 0.0133 |
| 512_051015-0.020 | positive | 0.0200 | 0.0163 |
| 512_051015-0.025 | positive | 0.0250 | 0.0235 |
| 512_051015-0.030 | positive | 0.0300 | 0.0311 |
| 512_051015-0.035 | positive | 0.0350 | 0.0322 |
| 512_051015-0.040 | positive | 0.0400 | 0.0330 |
| 512_051015-0.045 | positive | 0.0450 | 0.0371 |
| 512_051015-0.050 | positive | 0.0500 | 0.0296 |
| 512_051015-0.075 | positive | 0.0750 | 0.0388 |
| 512_051015-0.100 | positive | 0.1000 | 0.0500 |
| 512_051015-0.125 | positive | 0.1250 | 0.0682 |
| 512_051015-0.150 | positive | 0.1500 | 0.0881 |
| 512_051015-0.175 | positive | 0.1750 | 0.0743 |
| 512_051015-0.200 | positive | 0.2000 | 0.0948 |
| 512_051015-0.225 | positive | 0.2250 | 0.1386 |
| 512_051015-0.250 | positive | 0.2500 | 0.1762 |
| 512_051015-0.275 | positive | 0.2750 | 0.2095 |
| 512_051015-0.300 | positive | 0.3000 | 0.2319 |
| 51 2051 015-0.325 | positive | 0.3250 | 0.2451 |
| 512_051015-0.350 | positive | 0.3500 | 0.2761 |
| 512_051015-0.375 | positive | 0.3750 | 0.2971 |
| 512_051015-0.400 | positive | 0.4000 | 0.3063 |
| 512_051015-0.425 | positive | 0.4250 | 0.3056 |
| 512_051015-0.450 | positive | 0.4500 | 0.3568 |
| 512_051015-0.475 | positive | 0.4750 | 0.4021 |
| 512_051015-0.500 | positive | 0.5000 | 0.4009 |
| 512_051015-0.525 | positive | 0.5250 | 0.4008 |
| 512_051015-0.550 | positive | 0.5500 | 0.4460 |
| 512_051015-0.575 | positive | 0.5750 | 0.4464 |
| 512_051015-0.600 | positive | 0.6000 | 0.4736 |
| 512_051015-0.614 | positive | 0.6140 | 0.4474 |
| 512_130114-0.005 | positive | 0.0050 | 0.0136 |
| 512_130114-0.010 | positive | 0.0100 | 0.0167 |
| 512_130114-0.015 | positive | 0.0150 | 0.0198 |
| 512_130114-0.020 | positive | 0.0200 | 0.0363 |
| 512_130114-0.025 | positive | 0.0250 | 0.0352 |
| 512_130114-0.030 | positive | 0.0300 | 0.0402 |
| 512_130114 0.035 | positive | 0.0350 | 0.0285 |
| 512_130114-0.040 | positive | 0.0400 | 0.0309 |
| 512_130114-0.045 | positive | 0.0450 | 0.0374 |
| 512_130114-0.050 | positive | 0.0500 | 0.0406 |
| 512_130114-0.075 | positive | 0.0750 | 0.0492 |
| 512_130114-0.100 | positive | 0.1000 | 0.0904 |
| 512_130114-0.125 | positive | 0.1250 | 0.1008 |
| 512_130114-0.150 | positive | 0.1500 | 0.1432 |
| 512 130114-0.175 | positive | 0.1750 | 0.2025 |
| 512_130114-0.200 | positive | 0.2000 | 0.2242 |
| 512_130114-0.225 | positive | 0.2250 | 0.2376 |
| 512_130114-0.250 | positive | 0.2500 | 0.2570 |
| 512_130114-0.275 | positive | 0.2750 | 0.3262 |
| 512_130114-0.300 | positive | 0.3000 | 0.3659 |
| 512_130114-0.325 | positive | 0.3250 | 0.3947 |
| 512_130114-0.350 | positive | 0.3500 | 0.4564 |
| 512_130114-0.375 | positive | 0.3750 | 0.4838 |
| 512_130114-0.394 | positive | 0.3940 | 0.4822 |
| sub-healthy-1 | negative | 0.0000 | 0.0000 |
| sub-healthy-2 | negative | 0.0000 | 0.0024 |
| sub-healthy-3 | negative | 0.0000 | 0.0001 |
| sub-healthy-4 | negative | 0.0000 | 0.0005 |
| sub-healthy-5 | negative | 0.0000 | 0.0000 |
| sub-healthy-6 | negative | 0.0000 | 0.0162 |
| sub-healthy-7 | negative | 0.0000 | 0.0065 |
| sub-healthy-8 | negative | 0.0000 | 0.0000 |
| sub-healthy-9 | negative | 0.0000 | 0.0084 |
| sub-healthy-10 | negative | 0.0000 | 0.0071 |
| sub-healthy-11 | negative | 0.0000 | 0.0000 |
| sub-healthy-12 | negative | 0.0000 | 0.0124 |
| sub-healthy-13 | negative | 0.0000 | 0.0013 |
| sub-healthy-14 | negative | 0.0000 | 0.0118 |
| sub-healthy-15 | negative | 0.0000 | 0.0109 |
| sub-healthy-16 | negative | 0.0000 | 0.0069 |
| sub-healthy-17 | negative | 0.0000 | 0.0116 |
| sub-healthy-18 | negative | 0.0000 | 0.0076 |
| sub-healthy-19 | negative | 0.0000 | 0.0049 |
| sub-healthy-20 | negative | 0.0000 | 0.0000 |
| sub-healthy-21 | negative | 0.0000 | 0.0000 |
| sub-healthy-22 | negative | 0.0000 | 0.0095 |
| sub-healthy-23 | negative | 0.0000 | 0.0106 |
| sub-healthy-24 | negative | 0.0000 | 0.0121 |
| sub-healthy-25 | negative | 0.0000 | 0.0085 |
| sub-healthy-26 | negative | 0.0000 | 0.0000 |
| sub-healthy-27 | negative | 0.0000 | 0.0176 |
| sub-healthy-28 | negative | 0.0000 | 0.0099 |
| sub-healthy-29 | negative | 0.0000 | 0.0000 |
| sub-healthy-30 | negative | 0.0000 | 0.0076 |
| sub-healthy-31 | negative | 0.0000 | 0.0002 |
| sub-healthy-32 | negative | 0.0000 | 0.0076 |
| sub-healthy-33 | negative | 0.0000 | 0.0078 |
| sub-healthy-34 | negative | 0.0000 | 0.0071 |
| sub-healthy-35 | negative | 0.0000 | 0.0077 |
| sub-healthy-36 | negative | 0.0000 | 0.0110 |
| sub-healthy-37 | negative | 0.0000 | 0.0074 |
| sub-healthy-38 | negative | 0.0000 | 0.0000 |
| sub-healthy-39 | negative | 0.0000 | 0.0000 |
| sub-healthy-40 | negative | 0.0000 | 0.0105 |

TABLE S6
CRC plasma samples for Ip-WGS and targeted sequencing.

| | | | | | icharCNA- | | NDR- |
|---|---|---|---|---|---|---|---|
| sample | patient | sample | cancer | Ip-WGS | estimated | | estimated |
| name | ID | ID | type | coverage | ctDNA content | max VAF | ctDNA content |
| 069_020913 | 69 | CRC-13 | CRC | 1.97 | 0.00 | undetected | 0.00 |
| 069_310316 | 69 | CRC-14 | CRC | 7.01 | 0.09 | undetected | 0.08 |
| 095_100913 | 95 | CRC-15 | CRC | 5.77 | 0.38 | 0.54 | 0.37 |
| 095_150513 | 95 | CRC-16 | CRC | 6.18 | 0.43 | 0.55 | 0.47 |
| 1014_140616 | 1014 | CRC-17 | CRC | 1.82 | 0.39 | 0.56 | 0.38 |
| 1176_040815 | 1176 | CRC-18 | CRC | 2.96 | 0.05 | undetected | 0.21 |
| 1176_111116 | 1176 | CRC-19 | CRC | 1.35 | 0.00 | undetected | 0.06 |
| 1176_240715 | 1176 | CRC-20 | CRC | 1.27 | 0.00 | 0.11 | 0.06 |
| 1179_160316 | 1179 | CRC-21 | CRC | 1.96 | 0.16 | 0.29 | 0.25 |
| 1179_270715 | 1179 | CRC-22 | CRC | 3.78 | 0.18 | 0.20 | 0.34 |
| 1429_141116 | 1429 | CRC-23 | CRC | 0.42 | 0.20 | undetected | 0.08 |
| 1429_170316 | 1429 | CRC-24 | CRC | 5.83 | 0.39 | 0.36 | 0.36 |
| 1490_050516 | 1490 | CRC-25 | CRC | 2.12 | 0.49 | 0.72 | 0.49 |
| 1490_171116 | 1490 | CRC-26 | CRC | 2.08 | 0.00 | undetected | 0.33 |
| 149_100613 | 149 | CRC-27 | CRC | 2.43 | 0.00 | undetected | 0.19 |
| 149_130214 | 149 | CRC-28 | CRC | 3.72 | 0.05 | undetected | 0.18 |
| 1531_010716 | 1531 | CRC-29 | CRC | 1.38 | 0.48 | 0.77 | 0.49 |
| 1531_111016 | 1531 | CRC-30 | CRC | 4.23 | 0.00 | undetected | 0.07 |
| 1531_111116 | 1531 | CRC-31 | CRC | 1.93 | 0.00 | undetected | 0.00 |
| 330_091115 | 330 | CRC-32 | CRC | 5.93 | 0.23 | 0.38 | 0.25 |
| 330_170414 | 330 | CRC-33 | CRC | 6.93 | 0.00 | undetected | 0.00 |
| 357_110716 | 357 | CRC-34 | CRC | 3.48 | 0.51 | 0.56 | 0.49 |
| 357_230913 | 357 | CRC-35 | CRC | 2.06 | 0.00 | undetected | 0.11 |
| 357_290216 | 357 | CRC-36 | CRC | 4.28 | 0.57 | 0.49 | 0.39 |
| 357_291015 | 357 | CRC-37 | CRC | 5.94 | 0.37 | 0.29 | 0.38 |
| 375_021116 | 375 | CRC-38 | CRC | 3.13 | 0.00 | undetected | 0.10 |
| 375_210115 | 375 | CRC-39 | CRC | 2.90 | 0.00 | undetected | 0.17 |
| 386_091214 | 386 | CRC-40 | CRC | 0.70 | 0.09 | 0.22 | 0.21 |
| 386_180516 | 386 | CRC-41 | CRC | 3.86 | 0.32 | 0.49 | 0.38 |
| 476_050916 | 476 | CRC-42 | CRC | 1.09 | 0.11 | 0.19 | 0.09 |
| 476_110315 | 476 | CRC-43 | CRC | 3.32 | 0.12 | 0.27 | 0.20 |
| 519_020715 | 519 | CRC-44 | CRC | 6.30 | 0.15 | 0.17 | 0.29 |
| 519_140116 | 519 | CRC-45 | CRC | 2.31 | 0.06 | undetected | 0.14 |
| 519_240314 | 519 | CRC-46 | CRC | 4.88 | 0.29 | 0.51 | 0.33 |
| 571_261114 | 571 | CRC-47 | CRC | 2.64 | 0.00 | undetected | 0.06 |
| 571_291015 | 571 | CRC-48 | CRC | 2.07 | 0.05 | undetected | 0.15 |
| 575_191015 | 575 | CRC-49 | CRC | 2.63 | 0.11 | 0.12 | 0.10 |
| 575_270214 | 575 | CRC-50 | CRC | 1.40 | 0.19 | 0.11 | 0.18 |
| 575_270516 | 575 | CRC-51 | CRC | 2.13 | 0.00 | 0.11 | 0.11 |
| 575_270616 | 575 | CRC-52 | CRC | 6.88 | 0.05 | undetected | 0.21 |
| 592_171116 | 592 | CRC-53 | CRC | 2.42 | 0.00 | undetected | 0.08 |
| 592_280316 | 592 | CRC-54 | CRC | 3.01 | 0.00 | undetected | 0.09 |
| 741_091116 | 741 | CRC-55 | CRC | 1.50 | 0.00 | undetected | 0.07 |
| 741_150415 | 741 | CRC-56 | CRC | 4.26 | 0.00 | undetected | 0.10 |
| 834_090715 | 834 | CRC-57 | CRC | 2.71 | 0.00 | undetected | 0.00 |
| 834_241116 | 834 | CRC-58 | CRC | 4.41 | 0.13 | undetected | 0.09 |
| 836_090415 | 836 | CRC-59 | CRC | 1.27 | 0.00 | undetected | 0.04 |
| 836_090715 | 836 | CRC-60 | CRC | 4.39 | 0.00 | undetected | 0.17 |
| 836_180416 | 836 | CRC-61 | CRC | 4.23 | 0.39 | 0.25 | 0.22 |
| 897_200815 | 897 | CRC-62 | CRC | 6.55 | 0.34 | 0.47 | 0.26 |
| 897_220316 | 897 | CRC-63 | CRC | 8.03 | 0.07 | 0.16 | 0.07 |
| 986_060315 | 986 | CRC-64 | CRC | 6.37 | 0.19 | 0.19 | 0.31 |
| 986_260916 | 986 | CRC-65 | CRC | 4.52 | 0.28 | 0.73 | 0.47 |

TABLE S7
A panel of 100 genes frequently mutated
in colorectal and breast cancer.

ABL1
ACVR2A
AKT1
ALK
APC
ARID1A
ATM
ATR
AURKA
B2M
BCL9L
BRAF
BRCA1
BRCA2
CALR
CANX
CDH1
CDKN1B
CDKN2A
CHD4
CSF1R
CTCF
CTNNB1
DCC
DDR2
DMD
DOT1L
EGFR
EP300
ERBB2
ERBB3
ERBB4
ESR1
EZH2
FBXW7
FGFR1
FGFR2
FGFR3
FLT3
GATA3
GNA11
GNAQ
GNAS
HLA-A
HNF1A
HRAS
HSPA5
IDH1
IDH2
JAK1
JAK2
JAK3
KDR
KIT
KMT2C
KRAS
MAGI3
MAP2K1
MAP2K4
MAP3K1
MDM2
MDM4
MET
MLH1
MPL
MTOR
MUC17
NF1
NOTCH1
NOTCH4
NPM1
NRAS
PDGFRA
PDIA3
PIK3CA
PIK3R1
POLD1
POLD2
POLE
PTEN
PTPN11
RB1
RBM10
RET
RNF43
SMAD2
SMAD4
SMARCB1
SMO
SOX9
SRC
STK11
TAP1
TAPBP
TP53
TSC1
TSC2
VHL
ZFP36L2
ZNRF3

TABLE S8
Variant allele frequency estimation of CRC plasma samples.

| | | Pos | | | | | |
|---|---|---|---|---|---|---|---|
| Sample | Chr | (GRCh38) | Ref | Alt | Codon | AA | Gene |
| T095_100913 | chr12 | 25245350 | C | T | gGt/gAt | G/D | ENSG00000133703 |
| T095_100913 | chr17 | 72124084 | — | +C | —/C | —/X | ENSG00000125398 |
| T095_100913 | chr17 | 7674230 | C | T | Ggc/Agc | G/S | ENSG00000141510 |
| T095_150513 | chr12 | 25245350 | C | T | gGt/gAt | G/D | ENSG00000133703 |
| T095_150513 | chr17 | 72124084 | — | +C | —/C | —/X | ENSG00000125398 |
| T095_150513 | chr17 | 7674230 | C | T | Ggc/Agc | G/S | ENSG00000141510 |
| T1014_110115 | chr2 | 147926116- | — | −A | Aaa/aa | K/X | ENSG00000121989 |
| | | 7 | | | | | |
| T1014_140616 | chr14 | 104773077 | C | A | Gac/Tac | D/Y | ENSG00000142208 |
| T1014_140616 | chr17 | 72122793 | A | T | cAc/cTc | H/L | ENSG00000125398 |
| T1014_140616 | chr5 | 112839783 | G | T | Gag/Tag | E/* | ENSG00000134982 |
| T1014_180816 | chr14 | 104773077 | C | A | Gac/Tac | D/Y | ENSG00000142208 |
| T1014_180816 | chr17 | 72122793 | A | T | cAc/cTc | H/L | ENSG00000125398 |
| T1014_180816 | chr5 | 112839783 | G | T | Gag/Tag | E/* | ENSG00000134982 |
| T1176_240715 | chr3 | 142555897- | — | −T | Ata/ta | I/X | ENSG00000175054 |
| | | 8 | | | | | |
| T1179_160316 | chr12 | 25245351 | C | A | Ggt/Tgt | G/C | ENSG00000133703 |
| T1179_160316 | chr17 | 7674250 | C | A | tGt/tTt | C/F | ENSG00000141510 |
| T1179_160316 | chr4 | 152329715 | G | T | tCt/At | S/Y | ENSG00000109670 |
| T1179_160316 | chr5 | 112839826- | — | −T | agT/ag | S/X | ENSG00000134982 |
| | | 7 | | | | | |
| T1179_270715 | chr12 | 25245351 | C | A | Ggt/Tgt | G/C | ENSG00000133703 |
| T1179_270715 | chr17 | 7674250 | C | A | tGt/tTt | C/F | ENSG00000141510 |
| T1179_270715 | chr5 | 112839826- | — | −T | agT/ag | S/X | ENSG00000134982 |
| | | 7 | | | | | |
| T1279_221015 | chr10 | 87933148 | G | A | cGa/cAa | R/Q | ENSG00000171862 |
| T1279_221015 | chr6 | 112839240 | G | T | Gag/Tag | E/* | ENSG00000134982 |
| T1279_241116 | chr10 | 87933148 | G | A | cGa/cAa | R/Q | ENSG00000171862 |
| T1279_241116 | chr5 | 112839240 | G | T | Gag/Tag | E/* | ENSG00000134982 |
| T1429_141116 | chr1 | 114716126 | C | T | gGt/gAt | G/D | ENSG00000213281 |
| T1429_141116 | chr11 | 108297326 | A | G | aAt/aGt | N/S | ENSG00000149311 |
| T1429_141116 | chr12 | 132641691 | G | A | gcC/gcT | A | ENSG00000177084 |
| T1429_141116 | chr12 | 6581743 | C | T | aaG/aaA | K | ENSG00000111642 |
| T1429_141116 | chr13 | 32332343 | A | C | Aat/Cat | N/H | ENSG00000139618 |
| T1429_141116 | chr13 | 32332843 | A | G | tcA/tcG | S | ENSG00000139618 |
| T1429_141116 | chr13 | 32336584 | T | C | caT/caC | H | ENSG00000139618 |
| T1429_141116 | chr13 | 32337326 | A | G | Aac/Gac | N/D | ENSG00000139618 |
| T1429_141116 | chr17 | 43071077 | T | C | Agt/Ggt | S/G | ENSG00000012048 |
| T1429_141116 | chr17 | 43082453 | A | G | tcT/tcC | S | ENSG00000012048 |
| T1429_141116 | chr17 | 43091983 | T | C | aAa/aGa | K/R | ENSG00000012048 |
| T1429_141116 | chr17 | 43092418 | T | C | gAa/gGa | E/G | ENSG00000012048 |
| T1429_141116 | chr17 | 43093220 | A | G | Ttg/Ctg | L | ENSG00000012048 |
| T1429_141116 | chr17 | 43093449 | G | A | agC/agT | S | ENSG00000012048 |
| T1429_141116 | chr17 | 58362569 | C | T | cGg/cAg | R/Q | ENSG00000108375 |
| T1429_141116 | chr17 | 7675088 | C | T | cGc/cAc | R/H | ENSG00000141510 |
| T1429_141116 | chr4 | 54736599 | G | C | ctG/ctC | L | ENSG00000157404 |
| T1429_141116 | chr4 | 55113321 | G | A | aCa/aTa | T/I | ENSG00000128052 |
| T1429_141116 | chr6 | 32221046 | G | A | tCg/tTg | S/L | ENSG00000204301 |
| T1429_141116 | chr6 | 32847198 | T | C | gAt/gGc | D/G | ENSG00000168394 |
| T1429_170316 | chr1 | 114713908 | T | A | cAa/cTa | Q/L | ENSG00000213281 |
| T1429_170316 | chr10 | 8058488 | C | T | tCg/tTg | S/L | ENSG00000107485 |
| T1429_170316 | chr16 | 68815631 | T | C | gaT/gaC | D | ENSG00000039068 |
| T1429_170316 | chr17 | 7674230 | C | T | Ggc/Agc | G/S | ENSG00000141510 |
| T1429_170316 | chr22 | 29049985 | C | T | Cgg/Tgg | R/W | ENSG00000183579 |
| T1429_170316 | chr5 | 112792446 | C | T | Cga/Tga | R/* | ENSG00000134982 |
| T1490_050516 | chr1 | 11240392 | G | A | gCc/gTc | A/V | ENSG00000198793 |
| T1490_050516 | chr17 | 7675088 | C | T | cGc/cAc | R/H | ENSG00000141510 |
| T1490_050516 | chr19 | 2226938 | C | T | Cag/Tag | Q/* | ENSG00000104885 |
| T1490_050516 | chr4 | 55118645 | C | T | cGg/cAg | R/Q | ENSG00000128052 |
| T1490_050516 | chr5 | 112838070 | — | +TGAATACTACA | ttg/tTGAATACTACAtg | L/LNTTX | ENSG00000134982 |
| T1490_050516 | chr5 | 112839474 | C | T | Cag/Tag | Q/* | ENSG00000134982 |
| T1531_010716 | chr17 | 7675088 | C | T | cGc/cAc | R/H | ENSG00000141510 |
| T1531_010716 | chr2 | 43224452 | T | G | gAc/gCc | D/A | ENSG00000152518 |
| T1531_010716 | chr5 | 112839439 | C | A | tCa/tAa | S/* | ENSG00000134982 |
| T1531_160616 | chr17 | 7675088 | C | T | cGc/cAc | R/H | ENSG00000141510 |
| T1531_160616 | chr5 | 112839439 | C | A | tCa/tAa | S/* | ENSG00000134982 |
| T330_091115 | chr11 | 118908483 | — | +G | —/C | —/X | ENSG00000186174 |
| T330_091115 | chr19 | 50409156 | G | A | Gcg/Agc | G/R | ENSG00000062822 |
| T330_091115 | chr4 | 152326137 | G | A | Cgc/Tgc | R/C | ENSG00000109670 |
| T330_091115 | chr5 | 112839576 | C | T | Cag/Tag | Q/* | ENSG00000134S82 |
| T330_091115 | chr5 | 150089316 | C | T | Gtg/Atg | V/M | ENSG00000182578 |
| T357_110716 | chr12 | 25245350 | C | T | gGt/gAt | G/D | ENSG00000133703 |
| T357_110716 | chr13 | 32362670 | G | T | agG/agT | R/S | ENSG00000139618 |
| T357_110716 | chr17 | 7674945 | G | A | Cga/Tga | R/* | ENSG00000141510 |
| T357_110716 | chr2 | 211657762 | T | G | caA/caC | Q/H | ENSG00000178568 |
| T357_110716 | chr2 | 43224819- | — | −CGCGGCCGCCGCGGAGG | gCCTCCGCGGCGGCCGCG/g | ASAAAA/X | ENSG00000152518 |
| | | 36 | | | | | |
| T357_290216 | chr12 | 25245350 | C | T | gCt/gAt | G/D | ENSG00000133703 |
| T357_290216 | chr13 | 32362670 | G | T | agG/agT | R/S | ENSG00000139618 |
| T357_290216 | chr17 | 7674945 | G | A | Cga/Tga | R/* | ENSG00000141510 |
| T357_290216 | chr2 | 211657762 | T | G | caA/caC | Q/H | ENSG00000178568 |
| T357_290216 | chr2 | 43224819- | — | −CGCGGCCGCCGCGGAGG | gCCTCCGCGGCGGCCGCG/g | ASAAAA/X | ENSG00000152518 |
| | | 36 | | | | | |
| T357_291015 | chr12 | 25245350 | G | T | gGt/gAt | G/D | ENSG00000133703 |
| T357_291015 | chr13 | 32362670 | G | T | agG/agT | R/S | ENSG00000139618 |
| T357_291015 | chr17 | 7574945 | G | A | Cga/Tga | R/* | ENSG00000141510 |
| T357_291015 | chr2 | 211657762 | T | G | caA/caC | Q/H | ENSG00000178568 |
| T357_291015 | chr2 | 43224819- | — | −CGCGGCCGCCGCGGAGG | gCCTCCGCGGCGGCCGCG/g | ASAAAA/X | ENSG00000152518 |
| | | 36 | | | | | |
| T386_091214 | chr17 | 7675088 | C | T | cGc/cAc | R/H | ENSG00000141510 |
| T386_091214 | chr3 | 179218307 | A | C | cAg/cCg | Q/P | ENSG00000121879 |
| T386_091214 | chr5 | 112837893 | C | T | Cag/Tag | Q/* | ENSG00000134982 |
| T386_091214 | chrX | 33020185 | A | G | gTt/gCt | V/A | ENSG00000198947 |
| T386_180516 | chr10 | 121503937 | G | C | tCg/tGg | S/W | ENSG00000066468 |
| T386_180516 | chr12 | 112504716 | A | T | agA/agT | R/S | ENSG00000179295 |
| T386_180516 | chr17 | 7675088 | C | T | cGc/cAc | R/H | ENSG00000141510 |
| T386_180516 | chr3 | 179218307 | A | C | cAg/cCg | Q/P | ENSG00000121879 |
| T386_180516 | chr5 | 112837893 | C | T | Cag/Tag | Q/* | ENSG00000134982 |
| T386_180516 | chr5 | 112839543 | G | T | Gaa/Taa | E/* | ENSG00000134982 |
| T386_180516 | chr8 | 38421931 | A | T | gTt/gAt | V/D | ENSG00000077782 |
| T386_180516 | chrX | 33020185 | A | G | gTt/gCt | V/A | ENSG00000198947 |
| T476_050916 | chr17 | 7675088 | C | T | cGc/cAc | R/H | ENSG00000141510 |
| T476_050916 | chr2 | 43225692 | — | +C | —/G | —/X | ENSG00000152518 |
| T476_110315 | chr17 | 7675088 | C | T | cGc/cAc | R/H | ENSG00000141510 |
| T476_110315 | chr2 | 43225692 | — | +C | —/G | —/X | ENSG00000152518 |
| T476_110315 | chr3 | 179221102 | A | T | aAg/aTg | K/M | ENSG00000121879 |
| T512_051015 | chr15 | 43768557 | A | C | aAg/aCg | K/T | ENSG00000167004 |
| T512_051015 | chr18 | 51065534 | C | T | cCt/cTt | P/L | ENSG00000141646 |
| T512_051015 | chr5 | 112839729 | G | T | Gag/Tag | E/* | ENSG00000134982 |
| T512_130114 | chr18 | 51065534 | C | T | cCt/cTt | P/L | ENSG00000141646 |
| T512_130114 | chr5 | 112839729 | G | T | Gag/Tag | E/* | ENSG00000134982 |
| T519_020715 | chr17 | 7673803 | G | A | Cgt/Tgt | R/C | ENSG00000141510 |
| T519_210114 | chr17 | 7673803 | G | A | Cgt/Tgt | R/C | ENSG00000141510 |
| T519_210114 | chr20 | 58903555 | C | A | gaC/gaA | D/E | ENSG00000087460 |
| T519_210114 | chr5 | 112839465 | C | T | Cag/Tag | Q/* | ENSG00000134982 |
| T519_210114 | chr5 | 56871965 | G | T | Gaa/Taa | E/* | ENSG00000095015 |
| T519_240314 | chr17 | 7673803 | G | A | Cgt/Tgt | R/C | ENSG00000141510 |
| T519_240314 | chr20 | 58903555 | C | A | gaC/gaA | D/E | ENSG00000087460 |
| T519_240314 | chr5 | 112839465 | C | T | Cag/Tag | Q/* | ENSG00000134982 |
| T519_240314 | chr5 | 56871965 | G | T | Gaa/Taa | E/* | ENSG00000095015 |
| T575_191015 | chr17 | 7673811 | A | G | tTt/tCt | F/S | ENSG00000141510 |
| T575_191015 | chr3 | 142555897- | — | −T | Ata/ta | I/X | ENSG00000175054 |
| | | 8 | | | | | |
| T575_270214 | chr17 | 7673811 | A | G | tTt/tCt | F/S | ENSG00000141510 |
| T575_270516 | chr3 | 142555897- | — | −T | Ata/ta | I/K | ENSG00000175054 |
| | | 8 | | | | | |
| T809_030915 | chr1 | 64869346 | C | A | aaG/aaT | K/N | ENSG00000162434 |
| T809_030915 | chr17 | 7675088 | C | T | cGc/cAc | R/H | ENSG00000141510 |
| T809_030915 | chr5 | 112839693 | C | T | Cag/Tag | Q/* | ENSG00000134982 |
| T809_110914 | chr1 | 64869346 | C | A | aaG/aaT | K/N | ENSG00000162434 |
| T809_110914 | chr17 | 7675088 | C | T | cGc/cAc | R/H | ENSG00000141510 |
| T809_110914 | chr5 | 112839693 | C | T | Cag/Tag | Q/* | ENSG00000134982 |
| T836_180416 | chr17 | 7674939 | C | A | Gaa/Taa | E/* | ENSG00000141510 |
| T836_180416 | chr5 | 112839474 | C | T | Cag/Tag | Q/* | ENSG00000134982 |
| T897_200815 | chr12 | 25245347 | C | T | gGc/gAc | G/D | ENSG00000133703 |
| T897_200815 | chr17 | 72124097 | — | +CG | tcg/tCGcg | S/SX | ENSG00000125398 |
| T897_200815 | chr17 | 7675067- | — | −A | Tgc/gc | C/X | ENSG00000141510 |
| | | 8 | | | | | |
| T897_200815 | chr18 | 51065549 | G | A | cGc/cAc | R/H | ENSG00000141646 |
| T897_200815 | chr18 | 51078286 | A | T | gAt/gTt | D/V | ENSG00000141646 |
| T897_200815 | chr22 | 29050710 | C | T | ccC/ccT | P | ENSG00000183579 |
| T897_200815 | chr3 | 179199088 | G | A | cGa/cAa | R/Q | ENSG00000121879 |
| T897_200815 | chr4 | 162329695- | — | −T | aaA/aa | K/X | ENSG00000109670 |
| | | 8 | | | | | |
| T897_200815 | chr5 | 112839714 | G | T | Gaa/Taa | E/* | ENSG00000134982 |
| T897_220316 | chr5 | 112839714 | G | T | Gaa/Taa | E/* | ENSG00000134982 |
| T986_060315 | chr17 | 7675076 | T | C | cAt/cGt | H/R | ENSG00000141510 |
| T986_060315 | chr3 | 179218304 | A | G | gAg/gGg | E/G | ENSG00000121879 |
| T986_060315 | chr5 | 112792446 | C | T | Cga/Tga | R/* | ENSG00000134982 |
| T986_100215 | chr17 | 7675076 | T | C | cAt/cGt | H/R | ENSG00000141510 |
| T986_100215 | chr3 | 179218304 | A | G | gAg/gGg | E/G | ENSG00000121879 |
| T886_100215 | chr5 | 112792446 | C | T | Cga/Tga | R/* | ENSG00000134982 |
| T986_260916 | chr17 | 72123617 | — | +GA | cga/cGAga | R/RX | ENSG00000125398 |
| T986_260916 | chr17 | 7675076 | T | C | cA/cGt | H/R | ENSG00000141510 |
| T986_260916 | chr3 | 179218304 | A | G | gAg/gGg | E/G | ENSG00000121879 |
| T986_260916 | chr5 | 112792446 | C | T | Cga/Tga | R/* | ENSG00000134982 |
| T986_261016 | chr17 | 72123617 | — | +GA | cga/cGAga | R/RX | ENSG00000125398 |
| T986_261016 | chr17 | 7675076 | T | C | cAt/cGt | H/R | ENSG00000141510 |
| T986_261016 | chr3 | 178218304 | A | G | gAg/gGg | E/G | ENSG00000121879 |
| T986_261016 | chr5 | 112732446 | C | T | Cga/Tga | R/* | ENSG00000134982 |
| T986_261016 | chr7 | 55165435 | C | T | taC/taT | Y | ENSG00000416648 |

| | Symbol/ | | | mutect_VAF | varscan_VAF | mutect_VAF | varscan_VAF | |
|---|---|---|---|---|---|---|---|---|
| Sample | Gene | Feature | Exon | Tumour | Tumour | Normal | Normal | MedianVAF |
| T095_100913 | KRAS | ENST00000256078 | 2/6 | 0.211 | 0.212 | 0 | 0 | 0.2115 |
| T095_100913 | SOX9 | ENST00000245479 | 3/3 | | 0.202 | | 0 | 0.202 |
| T095_100913 | TP53 | ENST00000269305 | 7/11 | 0.539 | | 0 | | 0.539 |
| T095_150513 | KRAS | ENST00000256078 | 2/6 | 0.234 | 0.221 | 0 | 0 | 0.2275 |
| T095_150513 | SOX9 | ENST00000245479 | 3/3 | | 0.187 | | 0 | 0.187 |
| T095_150513 | TP53 | ENST00000269305 | 7/11 | 0.553 | | 0 | | 0.553 |
| T1014_110115 | ACVR2A | ENST00000241416 | 10/11 | | 0.285 | | 0.006 | 0.286 |
| T1014_140616 | AKT1 | ENST00000554581 | 10/13 | 0.199 | 0.199 | 0 | 0 | 0.199 |
| T1014_140616 | SOX9 | ENST00000245479 | 2/3 | 0.367 | | 0 | | 0.367 |
| T1014_140616 | ARC | ENST00000257430 | 16/16 | 0.559 | | 0 | | 0.559 |
| T1014_180816 | AKT1 | ENST00000554581 | 10/13 | 0.246 | 0.256 | 0 | 0 | 0.251 |
| T1014_180816 | SOX9 | ENST00000245479 | 2/3 | 0.305 | 0.284 | 0 | 0 | 0.2945 |
| T1014_180816 | APC | ENST00000257430 | 16/16 | 0.458 | | 0.002 | | 0.458 |
| T1176_240715 | ATR | ENST00000350721 | 10/47 | | 0.108 | | 0.037 | 0.108 |
| T1179_160316 | KRAS | ENST00000256076 | 2/6 | 0.289 | 0.288 | 0.001 | 0 | 0.2885 |
| T1179_160316 | TP53 | ENST00000269305 | 7/11 | 0.275 | 0.265 | 0 | 0.001 | 0.27 |
| T1179_160316 | FBXW7 | ENST00000281708 | 8/12 | 0.119 | 0.104 | 0 | 0 | 0.1115 |
| T1179_160316 | APC | ENST00000257430 | 16/16 | | 0.241 | | 0.001 | 0.241 |
| T1179_270715 | KRAS | ENST00000256078 | 2/6 | 0.203 | 0.198 | 0.001 | 0 | 0.2005 |
| T1179_270715 | TP53 | ENST00000269305 | 7/11 | 0.14 | 0.143 | 0 | 0.001 | 0.1415 |
| T1179_270715 | APC | ENST00000257430 | 16/16 | | 0.185 | | 0.001 | 0.185 |
| T1279_221015 | PTEN | ENST00000371953 | 5/9 | 0.515 | | 0 | | 0.615 |
| T1279_221015 | APC | ENST00000257430 | 16/16 | 0.54 | | 0.001 | | 0.64 |
| T1279_241116 | PTEN | ENST00000371953 | 5/9 | 0.378 | | 0 | | 0.378 |
| T1279_241116 | APC | ENST00000257430 | 16/16 | 0.447 | | 0.001 | | 0.447 |
| T1429_141116 | NRAS | ENST00000369535 | 2/7 | 0.108 | 0.135 | 0 | 0 | 0.1215 |
| T1429_141116 | ATM | ENST00000278616 | 33/63 | 0.465 | | 0 | | 0.465 |
| T1429_141116 | POLE | ENST00000320574 | 39/49 | 0.547 | | 0 | | 0.547 |
| T1429_141116 | CHD4 | ENST00000357008 | 31/40 | 0.46 | | 0.001 | | 0.45 |
| T1429_141116 | BRCA2 | ENST00000380152 | 10/27 | 0.403 | | 0 | | 0.403 |
| T1429_141116 | BRCA2 | ENST00000380152 | 10/27 | 0.41 | | 0 | | 0.41 |
| T1429_141116 | BRCA2 | ENST00000380152 | 11/27 | 0.465 | | 0 | | 0.465 |
| T1429_141116 | BRCA2 | ENST00000380152 | 11/27 | 0.451 | | 0 | | 0.451 |
| T1429_141116 | BRCA1 | ENST00000471181 | 16/24 | 0.521 | | 0.001 | | 0.621 |
| T1429_141116 | BRCA1 | ENST00000471181 | 12/24 | 0.456 | | 0 | | 0.456 |
| T1429_141116 | BRCA1 | ENST00000471181 | 10/24 | 0.495 | | 0.001 | | 0.495 |
| T1429_141116 | BRCA1 | ENST00000471181 | 10/24 | 0.461 | | 0.002 | | 0.461 |
| T1429_141116 | BRCA1 | ENST00000471181 | 10/24 | 0.489 | | 0.001 | | 0.489 |
| T1429_141116 | BRCA1 | ENST00000471181 | 10/24 | 0.483 | | 0.001 | | 0.483 |
| T1429_141116 | RNF43 | ENST00000584437 | 5/9 | 0.435 | | 0.001 | | 0.435 |
| T1429_141116 | TP53 | ENST00000269305 | 5/11 | | 0.109 | | 0.001 | 0.109 |
| T1429_141116 | KIT | ENST00000288135 | 18/21 | 0.443 | | 0 | | 0.443 |
| T1429_141116 | KDR | ENST00000263923 | 7/30 | 0.479 | | 0 | | 0.479 |
| T1429_141116 | NOTCH4 | ENST00000375023 | 4/30 | 0.35 | 0.341 | 0 | 0 | 0.3455 |
| T1429_141116 | TAP1 | ENST00000354258 | 10/11 | 0.661 | | 0.001 | | 0.661 |
| T1429_170316 | NRAS | ENST00000369535 | 3/7 | 0.348 | 0.322 | 0 | 0 | 0.335 |
| T1429_170316 | GATA3 | ENST00000379328 | 3/6 | 0.2 | 0.199 | 0 | 0 | 0.1995 |
| T1429_170316 | CDH1 | ENST00000261769 | 10/16 | 0.357 | | 0.001 | | 0.357 |
| T1429_170316 | TP53 | ENST00000269305 | 7/11 | 0.345 | 0.341 | 0 | 0 | 0.343 |
| T1429_170316 | ZNRF3 | ENST00000544604 | 8/9 | 0.233 | 0.232 | 0 | 0.001 | 0.2325 |
| T1429_170316 | APC | ENST00000267430 | 7/16 | 0.268 | 0.231 | 0 | 0 | 0.2495 |
| T1490_050516 | MTOR | ENST00000361445 | 11/58 | 0.164 | 0.145 | 0 | 0 | 0.1545 |
| T1490_050516 | TP53 | ENST00000269305 | 5/11 | 0.719 | | 0 | | 0.719 |
| T1490_050516 | DOT1L | ENST00000398665 | 27/28 | 0.24 | 0.267 | 0.001 | 0 | 0.2535 |
| T1490_050516 | KDR | ENST00000263923 | 3/30 | 0.362 | | 0 | | 0.362 |
| T1490_050516 | APC | ENST00000257430 | 16/16 | | 0.185 | | 0 | 0.185 |
| T1490_050516 | APC | ENST00000257430 | 16/16 | 0.526 | | 0 | | 0.526 |
| T1531_010716 | TP53 | ENST00000269305 | 5/11 | 0.747 | | 0.001 | | 0.747 |
| T1531_010716 | ZFP36L2 | ENST00000282388 | 2/2 | | 0.309 | | 0 | 0.309 |
| T1531_010716 | APC | ENST00000257430 | 16/16 | 0.77 | | 0 | | 0.77 |
| T1531_160616 | TP53 | ENST00000269305 | 6/11 | 0.788 | | 0 | | 0.788 |
| T1531_160616 | APC | ENST00000257430 | 16/16 | 0.79 | | 0 | | 0.79 |
| T330_091115 | BCL9L | ENST00000334801 | 2/8 | | 0.224 | | 0 | 0.224 |
| T330_091115 | POLD1 | ENST00000440232 | 16/27 | 0.259 | 0.256 | 0 | 0 | 0.2575 |
| T330_091115 | FBXW7 | ENST00000281708 | 10/12 | 0.382 | | 0 | | 0.382 |
| T330_091115 | APC | ENST00000267430 | 16/16 | 0.366 | | 0 | | 0.366 |
| T330_091115 | CSF1R | ENST00000286301 | 4/22 | 0.169 | 0.161 | 0.001 | 0 | 0.165 |
| T357_110716 | KRAS | ENST00000256078 | 2/6 | 0.496 | | 0 | | 0.496 |
| T357_110716 | BRCA2 | ENST00000380152 | 17/27 | 0.233 | 0.256 | 0 | 0 | 0.2445 |
| T357_110716 | TP53 | ENST00000269305 | 6/11 | 0.56 | | 0 | | 0.56 |
| T357_110716 | ER884 | ENST00000342788 | 16/28 | | 0.172 | | 0.001 | 0.172 |
| T357_110716 | ZFP36L2 | ENST00000282388 | 2/2 | | 0.324 | | 0.001 | 0.324 |
| T357_290216 | KRAS | ENST00000256078 | 2/6 | 0.494 | | 0 | | 0.494 |
| T357_290216 | BRCA2 | ENST00000380152 | 17/27 | 0.181 | 0.174 | 0 | 0 | 0.1775 |
| T357_290216 | TP53 | ENST00000269305 | 6/11 | 0.488 | | 0 | | 0.488 |
| T357_290216 | ER884 | ENST00000342788 | 16/28 | | 0.142 | | 0.001 | 0.142 |
| T357_290216 | 2FP36L2 | ENST00000282388 | 2/2 | | 0.271 | | 0.001 | 0.271 |
| T357_291015 | KRAS | ENST00000256078 | 2/6 | 0.291 | 0.297 | 0 | 0 | 0.294 |
| T357_291015 | BRCA2 | ENST00000360152 | 17/27 | 0.132 | 0.135 | 0 | 0 | 0.1335 |
| T357_291015 | TP53 | ENST00000269305 | 6/11 | 0.247 | 0.248 | 0 | 0 | 0.2475 |
| T357_291015 | ER884 | ENST00000342788 | 16/28 | | 0.106 | | 0.001 | 0.106 |
| T357_291015 | ZFP36L2 | ENST00000282388 | 2/2 | | 0.17 | | 0.001 | 0.17 |
| T386_091214 | TP53 | ENST00000269305 | 5/11 | 0.228 | 0.218 | 0.001 | 0.001 | 0.223 |
| T386_091214 | PIK3CA | ENST00000263967 | 10/21 | 0.154 | 0.15 | 0.001 | 0.001 | 0.152 |
| T386_091214 | APC | ENST00000257430 | 16/16 | 0.111 | 0.11 | 0.001 | 0 | 0.1105 |
| T386_091214 | DMD | ENST00000357033 | 2/79 | 0.11 | 0.113 | 0 | 0 | 0.1115 |
| T386_180516 | FGFR2 | ENST00000457416 | 10/18 | 0.135 | 0.177 | 0 | 0 | 0.156 |
| T386_180516 | PTPN11 | ENST00000351677 | 15/16 | 0.223 | 0.236 | 0 | 0 | 0.2295 |
| T386_180516 | TP53 | ENST00000269305 | 5/11 | 0.484 | | 0.001 | | 0.494 |
| T386_180516 | PIK3CA | ENST00000263967 | 10/21 | 0.384 | | 0.001 | | 0.384 |
| T386_180516 | APC | ENST00000257430 | 16/16 | 0.292 | 0.288 | 0.001 | 0 | 0.29 |
| T386_180516 | APC | ENST00000257430 | 16/16 | 0.23 | 0.244 | 0 | 0 | 0.237 |
| T386_180516 | FGFR1 | ENST00000425967 | 9/19 | 0.188 | 0.192 | 0 | 0 | 0.19 |
| T386_180516 | DMD | ENST00000357033 | 2/79 | 0.274 | 0.308 | 0 | 0 | 0.291 |
| T476_050916 | TP53 | ENST00000269305 | 5/11 | 0.183 | 0.195 | 0 | 0.001 | 0.189 |
| T476_050916 | ZFP36L2 | ENST00000282388 | 2/2 | | 0.176 | | 0 | 0.176 |
| T476_110315 | TP53 | ENST00000269305 | 5/11 | 0.243 | 0.264 | 0 | 0.001 | 0.2535 |
| T476_110315 | ZFP36L2 | ENST00000282388 | 2/2 | | 0.272 | | 0 | 0.272 |
| T476_110315 | PIK3CA | ENST00000263967 | 14/21 | 0.12 | 0.113 | 0 | 0 | 0.1165 |
| T512_051015 | PDIA3 | ENST00000300289 | 9/13 | 0.095 | 0.104 | 0 | 0 | 0.0995 |
| T512_051015 | SMAD4 | ENST00000342988 | 9/12 | 0.614 | | 0 | | 0.614 |
| T512_051015 | APC | ENST00000257430 | 16/16 | 0.684 | | 0 | | 0.684 |
| T512_130114 | SMAD4 | ENST00000342988 | 9/12 | 0.371 | | 0 | | 0.371 |
| T512_130114 | APC | ENST00000257430 | 16/16 | 0.464 | | 0 | | 0.464 |
| T519_020715 | TP53 | ENST00000269305 | 8/11 | 0.173 | 0.166 | 0 | 0 | 0.1695 |
| T519_210114 | TP53 | ENST00000269305 | 8/11 | 0.43 | | 0 | | 0.43 |
| T519_210114 | GNAS | ENST00000371100 | 4/13 | 0.111 | 0.12 | 0 | 0 | 0.1155 |
| T519_210114 | APC | ENST00000257430 | 16/16 | 0.195 | 0.189 | 0 | 0.001 | 0.192 |
| T519_210114 | MAP3K1 | ENST00000399503 | 7/20 | 0.192 | 0.194 | 0 | 0 | 0.193 |
| T519_240314 | TP53 | ENST00000269305 | 8/11 | 0.513 | | 0 | | 0.613 |
| T519_240314 | GNAS | ENST00000371100 | 4/13 | 0.12 | 0.109 | 0 | 0 | 0.1145 |
| T519_240314 | APC | ENST00000257430 | 16/16 | 0.215 | 0.223 | 0 | 0.001 | 0.219 |
| T519_240314 | MAP3K1 | ENST00000399503 | 7/20 | 0.205 | 0.206 | 0 | 0 | 0.205 |
| T575_191015 | TP53 | ENST00000269305 | 8/11 | 0.104 | 0.103 | 0.001 | 0 | 0.1035 |
| T575_191015 | ATR | ENST00000350721 | 10/47 | | 0.116 | | 0.028 | 0.116 |
| T575_270214 | TP53 | ENST00000269305 | 8/11 | 0.109 | 0.107 | 0.001 | 0 | 0.108 |
| T575_270516 | ATR | ENST00000359721 | 10/47 | | 0.109 | | 0.028 | 0.109 |
| T809_030915 | JAK1 | ENST00000342505 | 6/25 | 0.123 | 0.135 | 0 | 0 | 0.129 |
| T809_030915 | TP53 | ENST00000269305 | 5/11 | 0.544 | | 0 | | 0.544 |
| T809_030915 | APC | ENST00000257430 | 16/16 | 0.542 | | 0.001 | | 0.542 |
| T809_110914 | JAK1 | ENST00000342505 | 5/25 | 0.186 | 0.172 | 0 | 0 | 0.179 |
| T809_110914 | TP53 | ENST00000269305 | 5/11 | 0.727 | | 0 | | 0.727 |
| T809_110914 | APC | ENST00000257430 | 16/16 | 0.744 | | 0.001 | | 0.744 |
| T836_180416 | TP53 | ENST00000269305 | 6/11 | 0.224 | 0.222 | 0 | 0 | 0.223 |
| T836_180416 | APC | ENST00000257430 | 16/16 | 0.248 | 0.259 | 0 | 0 | 0.2535 |
| T897_200815 | KRAS | ENST00000256078 | 2/6 | 0.171 | 0.17 | 0 | 0 | 0.1705 |
| T897_200815 | SOX9 | ENST00000245479 | 3/3 | | 0.213 | | 0 | 0.213 |
| T897_200815 | TP53 | ENST00000269305 | 5/11 | | 0.327 | | 0.001 | 0.327 |
| T897_200815 | SMAD4 | ENST00000342988 | 9/12 | 0.128 | 0.116 | 0 | 0.001 | 0.122 |
| T897_200815 | SMAD4 | ENST00000342988 | 12/12 | 0.236 | 0.229 | 0.001 | 0 | 0.2325 |
| T897_200815 | ZNRF3 | ENST00000544604 | 8/9 | 0.204 | 0.229 | 0 | 0 | 0.2165 |
| T897_200815 | PIK3CA | ENST00000263967 | 2/21 | 0.286 | 0.304 | 0 | 0.001 | 0.296 |
| T897_200815 | FBXW7 | ENST00000281708 | 8/12 | | 0.214 | | 0 | 0.214 |
| T897_200815 | APC | ENST00000257430 | 16/16 | 0.467 | | 0 | | 0.467 |
| T897_220316 | APC | ENST00000257430 | 16/16 | 0.173 | 0.156 | 0 | 0.001 | 0.1646 |
| T986_060315 | TP53 | ENST00000269305 | 5/11 | 0.189 | 0.198 | 0.001 | 0.001 | 0.1936 |
| T986_060315 | PIK3CA | ENST00000263967 | 10/21 | 0.164 | 0.186 | 0 | 0 | 0.175 |
| T986_060315 | APC | ENST00000257430 | 7/16 | 0.102 | 0.107 | 0 | 0.001 | 0.1046 |
| T986_100215 | TP53 | ENST00000269306 | 5/11 | 0.306 | 0.316 | 0.001 | 0.001 | 0.31 |
| T986_100215 | PIK3CA | ENST00000263967 | 10/21 | 0.232 | 0.222 | 0 | 0 | 0.227 |
| T886_100215 | APC | ENST00000257430 | 7/16 | 0.179 | 0.174 | 0 | 0.001 | 0.1765 |
| T986_260916 | SOX9 | ENST00000245479 | 3/3 | | 0.311 | | 0.001 | 0.311 |
| T986_260916 | TP53 | ENST00000269305 | 5/11 | 0.726 | | 0.001 | | 0.726 |
| T986_260916 | PIK3CA | ENST00000263967 | 10/21 | 0.65 | | 0 | | 0.65 |
| T986_260916 | APC | ENST00000257430 | 7/16 | 0.559 | | 0 | | 0.569 |
| T986_261016 | SOX9 | ENST00000245479 | 3/3 | | 0.206 | | 0.001 | 0.208 |
| T986_261016 | TP53 | ENST00000269305 | 5/11 | 0.413 | | 0.001 | | 0.413 |
| T986_261016 | PIK3CA | ENST00000263967 | 10/21 | 0.361 | | 0 | | 0.351 |
| T986_261016 | APC | ENST00000257430 | 7/16 | 0.261 | 0.268 | 0 | 0.001 | 0.2645 |
| T986_261016 | EGFR | ENST00000275493 | 15/28 | 0.164 | 0.164 | 0 | 0 | 0.1625 |

TABLE S9
Mutations missed by the callers for the CRC patients with serial plasma samples.

| | | | | | | | | | | | ctDNA | ctDNA |
|---|---|---|---|---|---|---|---|---|---|---|---|---|
| | | | | Pos | | | Symbol/ | VAF- | VAF- | VAF- | detection - | detection - |
| Patient | Sample | Day | chr | (GRCh38) | Ref | Alt | Gene | Mutect | Varscan | manual | ichorCNA | NDR |
| 357 | 357_230913 | 34 | chr13 | 32362670 | G | T | BRCA2 | undetected | undetected | 0 | negative | positive |
| | | | chr2 | 211657762 | T | G | ERB84 | undetected | undetected | 0 | | |
| | | | chr12 | 25245350 | C | T | KRAS | undetected | undetected | 0.0105 | | |
| | | | chr17 | 7674945 | G | A | TP53 | undetected | undetected | 0 | | |
| | | | chr2 | 43224819-36 | — | — | ZFP36L2 | undetected | undetected | 0 | | |
| | | | | | | CGCGGC | | | | | | |
| | | | | | | CGCCGC | | | | | | |
| | | | | | | GGAGG | | | | | | |
| 1531 | 1531_111016 | 160 | chr5 | 112839439 | C | A | APC | undetected | undetected | 0 | negative | positive |
| | | | chr17 | 7675088 | C | T | TP53 | undetected | undetected | 0.0012 | | |
| 1531 | 1531_111116 | 191 | chr5 | 112839439 | C | A | APC | undetected | undetected | 0 | negative | negative |
| | | | chr17 | 7675088 | C | T | TP53 | undetected | undetected | 0 | | |
| 575 | 575_270616 | 864 | chr3 | 142555897-8 | — | −T | ATR | undetected | undetected | 0.0579 | positive | positive |
| | | | chr17 | 7673811 | A | G | TP53 | undetected | undetected | 0.0546 | | |
| 519 | 519_140116 | 726 | chr5 | 112839465 | C | T | APC | undetected | undetected | 0.0247 | positive | positive |
| | | | chr20 | 58903555 | C | A | GNAS | undetected | undetected | 0.0232 | | |
| | | | chr5 | 56871965 | G | T | MAP3K1 | undetected | undetected | 0.0177 | | |
| | | | chr17 | 7673803 | G | A | TP53 | undetected | undetected | 0.0448 | | |

TABLE S10
Information on all candidate pan-cancer features of nucleosome-depleted regions.

| ID | Transcript | Gene | Chr | Site | Region | Group | FPKM_{blood} |
|---|---|---|---|---|---|---|---|
| 1 | ENST00000008938.4 | PGLYRP1 | 19 | 46526323 | promoter | blood | 148.76 |
| 2 | ENST00000177694.1 | TBX21 | 17 | 45810610 | promoter | blood | 5.98 |
| 3 | ENST00000194097.8 | NAIP | 5 | 70316737 | promoter | blood | 10.22 |
| 4 | ENST00000199708.2 | HBQ1 | 16 | 230452 | promoter | blood | 45.79 |
| 5 | ENST00000217133.1 | TUBB1 | 20 | 57594309 | promoter | blood | 6.01 |
| 6 | ENST00000219596.5 | MEFV | 16 | 3306627 | promoter | blood | 19.19 |
| 7 | ENST00000221804.4 | CLC | 19 | 40228668 | promoter | blood | 19.65 |
| 8 | ENST00000221954.6 | CEACAM4 | 19 | 42133442 | promoter | blood | 32.81 |
| 9 | ENST00000225275.3 | MPO | 17 | 56358296 | promoter | blood | 7.23 |
| 10 | ENST00000233997.3 | AZU1 | 19 | 827836 | promoter | blood | 14.03 |
| 11 | ENST00000234347.9 | PRTN3 | 19 | 840960 | promoter | blood | 13.78 |
| 12 | ENST00000236826.7 | MMP8 | 11 | 102595685 | promoter | blood | 20.68 |
| 13 | ENST00000245620.13 | LILRB3 | 19 | 54726850 | promoter | blood | 22.83 |
| 14 | ENST00000258104.7 | DYSF | 2 | 71680852 | promoter | blood | 6.76 |
| 15 | ENST00000262407.5 | ITGA2B | 17 | 42466873 | promoter | blood | 12.34 |
| 16 | ENST00000262651.3 | HCK | 20 | 30640045 | promoter | blood | 8.74 |
| 17 | ENST00000262865.8 | BPI | 20 | 36932525 | promoter | blood | 18.06 |
| 18 | ENST00000263621.1 | ELANE | 19 | 852291 | promoter | blood | 21.67 |
| 19 | ENST00000264260.6 | IL18RAP | 2 | 103035149 | promoter | blood | 42.11 |
| 20 | ENST00000264834.4 | KLF1 | 19 | 12997995 | promoter | blood | 8.95 |
| 21 | ENST00000267396.8 | REM2 | 14 | 23352374 | promoter | blood | 6.91 |
| 22 | ENST00000287497.12 | ITGAM | 16 | 31271311 | promoter | blood | 6.52 |
| 23 | ENST00000294800.7 | FCGR3B | 1 | 161601252 | promoter | blood | 13.85 |
| 24 | ENST00000295619.3 | PROK2 | 3 | 71834212 | promoter | blood | 12.98 |
| 25 | ENST00000295683.2 | CXCR1 | 2 | 219031718 | promoter | blood | 355.57 |
| 26 | ENST00000296028.3 | PPBP | 4 | 74853914 | promoter | blood | 50.15 |
| 27 | ENST00000296435.2 | CAMP | 3 | 48264837 | promoter | blood | 27.17 |
| 28 | ENST00000297435.2 | DEFA4 | 8 | 6795860 | promoter | blood | 34.98 |
| 29 | ENST00000299663.7 | CLEC4E | 12 | 8693559 | promoter | blood | 29.27 |
| 30 | ENST00000299665.2 | CLEC4D | 12 | 8666136 | promoter | blood | 10.47 |
| 31 | ENST00000302312.8 | AHSP | 16 | 31539185 | promoter | blood | 85.51 |
| 32 | ENST00000304076.6 | VAV1 | 19 | 6772725 | promoter | blood | 7.61 |
| 33 | ENST00000304361.8 | CLEC12A | 12 | 10124014 | promoter | blood | 10.23 |
| 34 | ENST00000307395.4 | GP9 | 3 | 128779610 | promoter | blood | 9.72 |
| 35 | ENST00000307564.8 | AKNA | 9 | 117156685 | promoter | blood | 11.95 |
| 36 | ENST00000310544.8 | PHOSPHO1 | 17 | 47307890 | promoter | blood | 30.92 |
| 37 | ENST00000312156.8 | NFE2 | 12 | 54689544 | promoter | blood | 11.15 |
| 38 | ENST00000314412.6 | FUT7 | 9 | 139927462 | promoter | blood | 16.90 |
| 39 | ENST00000314446.9 | LILRB2 | 19 | 54785039 | promoter | blood | 7.87 |
| 40 | ENST00000318507.6 | CXCR2 | 2 | 218990727 | promoter | blood | 157.27 |
| 41 | ENST00000324134.10 | NLRP12 | 19 | 54327597 | promoter | blood | 9.17 |
| 42 | ENST00000326965.6 | TAGAP | 6 | 159466184 | promoter | blood | 5.94 |
| 43 | ENST00000327857.6 | DEFA3 | 8 | 6875823 | promoter | blood | 355.10 |
| 44 | ENST00000329800.7 | TMCC2 | 1 | 205225329 | promoter | blood | 10.64 |
| 45 | ENST00000330597.3 | HBG1 | 11 | 5271122 | promoter | blood | 5.07 |
| 46 | ENST00000330807.9 | ALAS2 | X | 55057497 | promoter | blood | 142.66 |
| 47 | ENST00000331941.6 | CSF3R | 1 | 36945120 | promoter | blood | 16.12 |
| 48 | ENST00000335854.8 | ALAS2 | X | 55057410 | promoter | blood | 101.19 |
| 49 | ENST00000336906.4 | HBG2 | 11 | 5276041 | promoter | blood | 104.85 |
| 50 | ENST00000338372.6 | VSTM1 | 19 | 54567207 | promoter | blood | 10.71 |
| 51 | ENST00000340023.6 | FPR2 | 19 | 52264104 | promoter | blood | 34.71 |
| 52 | ENST00000342063.4 | C19orf35 | 19 | 2282175 | promoter | blood | 8.64 |
| 53 | ENST00000342571.7 | MKNK1 | 1 | 47051643 | promoter | blood | 14.42 |
| 54 | ENST00000344138.8 | GRAP2 | 22 | 40297086 | promoter | blood | 5.60 |
| 55 | ENST00000348343.10 | BMX | X | 15518952 | promoter | blood | 6.99 |
| 56 | ENST00000353065.7 | PROK2 | 3 | 71834357 | promoter | blood | 82.89 |
| 57 | ENST00000355524.7 | FCAR | 19 | 55385736 | promoter | blood | 53.04 |
| 58 | ENST00000356815.3 | HBM | 16 | 215977 | promoter | blood | 163.82 |
| 59 | ENST00000357396.7 | CEACAM3 | 19 | 42300369 | promoter | blood | 16.26 |
| 60 | ENST00000367053.5 | CR1 | 1 | 207669502 | promoter | blood | 10.47 |
| 61 | ENST00000367964.6 | FCGR3B | 1 | 161601753 | promoter | blood | 465.71 |
| 62 | ENST00000368034.8 | CD244 | 1 | 160832645 | promoter | blood | 6.27 |
| 63 | ENST00000373103.5 | CSF3R | 1 | 36948879 | promoter | blood | 61.20 |
| 64 | ENST00000375448.4 | PADI4 | 1 | 17634692 | promoter | blood | 95.04 |
| 65 | ENST00000375809.7 | C6orf25 | 6 | 31691150 | promoter | blood | 5.36 |
| 66 | ENST00000375862.6 | HCK | 20 | 30640000 | promoter | blood | 20.11 |
| 67 | ENST00000376092.7 | LST1 | 6 | 31554977 | promoter | blood | 5.01 |
| 68 | ENST00000376670.7 | GATA1 | X | 48644962 | promoter | blood | 11.17 |
| 69 | ENST00000377901.8 | TMEM71 | 8 | 133772828 | promoter | blood | 6.74 |
| 70 | ENST00000378023.8 | FAM65B | 6 | 24877583 | promoter | blood | 15.67 |
| 71 | ENST00000378625.5 | NADK | 1 | 1711508 | promoter | blood | 13.09 |
| 72 | ENST00000379595.7 | PSTPIP1 | 15 | 77287472 | promoter | blood | 5.90 |
| 73 | ENST00000380299.3 | HBD | 11 | 5255878 | promoter | blood | 138.84 |
| 74 | ENST00000380315.2 | HBB | 11 | 5250625 | promoter | blood | 5.45 |
| 75 | ENST00000381153.7 | C11orf21 | 11 | 2323290 | promoter | blood | 11.33 |
| 76 | ENST00000381603.7 | SIRPB1 | 20 | 1600642 | promoter | blood | 26.01 |
| 77 | ENST00000381605.8 | SIRPB1 | 20 | 1600655 | promoter | blood | 24.72 |
| 78 | ENST00000382689.7 | DEFA1B | 8 | 6856717 | promoter | blood | 118.00 |
| 79 | ENST00000382692.2 | DEFA1 | 8 | 6837602 | promoter | blood | 996.76 |
| 80 | ENST00000391726.7 | FCAR | 19 | 55385704 | promoter | blood | 6.00 |
| 81 | ENST00000392619.1 | CD300E | 17 | 72619877 | promoter | blood | 24.98 |
| 82 | ENST00000392621.5 | CD300LB | 17 | 72527605 | promoter | blood | 7.72 |
| 83 | ENST00000392841.1 | HMBS | 11 | 118958697 | promoter | blood | 7.28 |
| 84 | ENST00000393450.5 | MYL4 | 17 | 45286714 | promoter | blood | 22.77 |
| 85 | ENST00000394430.5 | RASGRP2 | 11 | 64512061 | promoter | blood | 17.61 |
| 86 | ENST00000394432.7 | RASGRP2 | 11 | 64511650 | promoter | blood | 9.91 |
| 87 | ENST00000397497.8 | PTK2B | 8 | 27288416 | promoter | blood | 5.81 |
| 88 | ENST00000397797.1 | HBA1 | 16 | 226703 | promoter | blood | 25.27 |
| 89 | ENST00000398421.6 | NCF1 | 7 | 74188358 | promoter | blood | 11.04 |
| 90 | ENST00000398632.3 | MX2 | 21 | 42774561 | promoter | blood | 7.28 |
| 91 | ENST00000399753.2 | MSRB1 | 16 | 1993196 | promoter | blood | 11.22 |
| 92 | ENST00000402255.5 | APOBEC3A | 22 | 39348756 | promoter | blood | 11.96 |
| 93 | ENST00000402280.5 | NLRC4 | 2 | 32489949 | promoter | blood | 5.84 |
| 94 | ENST00000404054.3 | TACC3 | 4 | 1741078 | promoter | blood | 5.17 |
| 95 | ENST00000409369.1 | IL18RAP | 2 | 103036146 | promoter | blood | 46.86 |
| 96 | ENST00000410026.6 | NABP1 | 2 | 192542862 | promoter | blood | 7.35 |
| 97 | ENST00000413580.5 | PHOSPHO1 | 17 | 47308128 | promoter | blood | 42.76 |
| 98 | ENST00000414723.1 | GCA | 2 | 163212948 | promoter | blood | 9.14 |
| 99 | ENST00000415495.5 | CEACAM3 | 19 | 42300549 | promoter | blood | 14.84 |
| 100 | ENST00000418507.6 | LST1 | 6 | 31553901 | promoter | blood | 6.17 |
| 101 | ENST00000421556.5 | RASGRP2 | 11 | 64511594 | promoter | blood | 5.27 |
| 102 | ENST00000421702.2 | FCGR3B | 1 | 161600948 | promoter | blood | 18.36 |
| 103 | ENST00000425417.1 | ARHGAP26 | 5 | 142586765 | promoter | blood | 5.74 |
| 104 | ENST00000425515.6 | VNN3 | 6 | 133055904 | promoter | blood | 8.73 |
| 105 | ENST00000426897.6 | CD37 | 19 | 49838653 | promoter | blood | 7.43 |
| 106 | ENST00000427321.1 | C1orf228 | 1 | 45189457 | promoter | blood | 8.41 |
| 107 | ENST00000427466.1 | THEMIS2 | 1 | 28199067 | promoter | blood | 7.94 |
| 108 | ENST00000430041.6 | ARHGAP9 | 12 | 57871981 | promoter | blood | 22.12 |
| 109 | ENST00000436504.2 | LILRB3 | 19 | 54723224 | promoter | blood | 69.95 |
| 110 | ENST00000436693.6 | TLR6 | 4 | 38858438 | promoter | blood | 5.83 |
| 111 | ENST00000441002.1 | IL1R2 | 2 | 102624977 | promoter | blood | 16.30 |
| 112 | ENST00000441045.5 | NAMPT | 7 | 105925397 | promoter | blood | 5.42 |
| 113 | ENST00000441366.6 | EPB42 | 15 | 43513249 | promoter | blood | 11.69 |
| 114 | ENST00000442948.3 | FOLR3 | 11 | 71846989 | promoter | blood | 9.64 |
| 115 | ENST00000444257.5 | PLB1 | 2 | 28827594 | promoter | blood | 7.51 |
| 116 | ENST00000445347.1 | LILRB3 | 19 | 54726959 | promoter | blood | 49.26 |
| 117 | ENST00000448367.5 | FES | 15 | 91432780 | promoter | blood | 6.30 |
| 118 | ENST00000452208.1 | RGL4 | 22 | 24038671 | promoter | blood | 64.94 |
| 119 | ENST00000453777.1 | LILRA1 | 19 | 55105125 | promoter | blood | 6.64 |
| 120 | ENST00000455062.2 | NCF1 | 7 | 74191615 | promoter | blood | 15.11 |
| 121 | ENST00000456013.5 | PTPN6 | 12 | 7060434 | promoter | blood | 7.47 |
| 122 | ENST00000460045.1 | IMPDH1 | 7 | 128035225 | promoter | blood | 8.72 |
| 123 | ENST00000460208.1 | LILRB3 | 19 | 54721567 | promoter | blood | 51.15 |
| 124 | ENST00000460442.5 | ZDHHC19 | 3 | 195925781 | promoter | blood | 22.43 |
| 125 | ENST00000460650.5 | SELL | 1 | 169673894 | promoter | blood | 5.03 |
| 126 | ENST00000462275.5 | TBXAS1 | 7 | 139529072 | promoter | blood | 6.07 |
| 127 | ENST00000462927.5 | CYTH4 | 22 | 37678566 | promoter | blood | 26.95 |
| 128 | ENST00000463153.1 | ABHD5 | 3 | 43759160 | promoter | blood | 5.85 |
| 129 | ENST00000463516.5 | MYO1G | 7 | 45014814 | promoter | blood | 5.23 |
| 130 | ENST00000464329.1 | CD53 | 1 | 111440292 | promoter | blood | 10.28 |
| 131 | ENST00000464526.1 | LST1 | 6 | 31553971 | promoter | blood | 9.98 |
| 132 | ENST00000464591.1 | C1orf162 | 1 | 112017190 | promoter | blood | 7.62 |
| 133 | ENST00000464994.5 | IL1R2 | 2 | 102608422 | promoter | blood | 12.24 |
| 134 | ENST00000465814.5 | ARAP1 | 11 | 72433133 | promoter | blood | 10.09 |
| 135 | ENST00000465984.5 | SLC11A1 | 2 | 219246911 | promoter | blood | 10.54 |
| 136 | ENST00000466138.1 | CSF3R | 1 | 36933410 | promoter | blood | 32.37 |
| 137 | ENST00000466151.1 | PADI2 | 1 | 17409921 | promoter | blood | 7.13 |
| 138 | ENST00000466612.5 | ABTB1 | 3 | 127391857 | promoter | blood | 6.18 |
| 139 | ENST00000467001.1 | PADI4 | 1 | 17675225 | promoter | blood | 24.03 |
| 140 | ENST00000467525.5 | FCGR2A | 1 | 161476123 | promoter | blood | 8.40 |
| 141 | ENST00000467786.1 | FGD3 | 9 | 95737532 | promoter | blood | 43.15 |
| 142 | ENST00000467972.5 | SLC12A9 | 7 | 100454416 | promoter | blood | 6.80 |
| 143 | ENST00000468221.5 | SLC11A1 | 2 | 219246752 | promoter | blood | 42.34 |
| 144 | ENST00000468945.1 | PADI4 | 1 | 17672529 | promoter | blood | 17.46 |
| 145 | ENST00000469273.1 | LILRB3 | 19 | 54724660 | promoter | blood | 16.30 |
| 146 | ENST00000469280.1 | NCF2 | 1 | 183533186 | promoter | blood | 13.91 |
| 147 | ENST00000469349.1 | NBEAL2 | 3 | 47049253 | promoter | blood | 7.54 |
| 148 | ENST00000469380.1 | CSF3R | 1 | 36939431 | promoter | blood | 5.77 |
| 149 | ENST00000469449.1 | SLC11A1 | 2 | 219248410 | promoter | blood | 5.27 |
| 150 | ENST00000469532.1 | RAC2 | 22 | 37640318 | promoter | blood | 9.83 |
| 151 | ENST00000469799.5 | SLC11A1 | 2 | 219247002 | promoter | blood | 8.27 |
| 152 | ENST00000469886.5 | CYTH4 | 22 | 37678556 | promoter | blood | 6.16 |
| 153 | ENST00000470579.5 | ARID5A | 2 | 97202480 | promoter | blood | 7.93 |
| 154 | ENST00000471466.5 | RAB24 | 5 | 176730706 | promoter | blood | 6.58 |
| 155 | ENST00000471836.1 | PARVG | 22 | 44577630 | promoter | blood | 6.53 |
| 156 | ENST00000472694.1 | HBA1 | 16 | 226697 | promoter | blood | 64.74 |
| 157 | ENST00000473070.1 | FAM65B | 6 | 24843364 | promoter | blood | 19.03 |
| 158 | ENST00000473156.5 | LILRA1 | 19 | 55105047 | promoter | blood | 7.68 |
| 159 | ENST00000473883.5 | HCLS1 | 3 | 121363030 | promoter | blood | 36.16 |
| 160 | ENST00000474129.1 | ABTB1 | 3 | 127395815 | promoter | blood | 8.59 |
| 161 | ENST00000475225.5 | SLC11A1 | 2 | 219247025 | promoter | blood | 37.49 |
| 162 | ENST00000475472.5 | FGR | 1 | 27961645 | promoter | blood | 49.95 |
| 163 | ENST00000475683.1 | TUBA4A | 2 | 220118638 | promoter | blood | 10.51 |
| 164 | ENST00000476050.5 | TRAF3IP3 | 1 | 209941940 | promoter | blood | 5.61 |
| 165 | ENST00000476408.1 | CD53 | 1 | 111422859 | promoter | blood | 10.54 |
| 166 | ENST00000476492.1 | LAPTM5 | 1 | 31230604 | promoter | blood | 6.02 |
| 167 | ENST00000476532.1 | SASH3 | X | 128913955 | promoter | blood | 9.72 |
| 168 | ENST00000477720.1 | LILRA5 | 19 | 54822933 | promoter | blood | 16.70 |
| 169 | ENST00000478298.5 | ABTB1 | 3 | 127395379 | promoter | blood | 6.71 |
| 170 | ENST00000478902.1 | ZDHHC18 | 1 | 27177620 | promoter | blood | 12.52 |
| 171 | ENST00000479534.5 | PADI2 | 1 | 17401846 | promoter | blood | 5.78 |
| 172 | ENST00000479996.1 | RGS19 | 20 | 62710778 | promoter | blood | 9.02 |
| 173 | ENST00000480467.5 | CDK5RAP2 | 9 | 123165773 | promoter | blood | 16.88 |
| 174 | ENST00000480825.6 | CSF3R | 1 | 36941928 | promoter | blood | 127.36 |
| 175 | ENST00000481215.1 | RAC2 | 22 | 37627543 | promoter | blood | 7.04 |
| 176 | ENST00000481314.5 | HCLS1 | 3 | 121379748 | promoter | blood | 13.32 |
| 177 | ENST00000481517.1 | STAT5B | 17 | 40369889 | promoter | blood | 8.48 |
| 178 | ENST00000481789.1 | KCNAB2 | 1 | 6156316 | promoter | blood | 8.66 |
| 179 | ENST00000482429.1 | LTB | 6 | 31550183 | promoter | blood | 5.85 |
| 180 | ENST00000482543.1 | PTK2B | 8 | 27310374 | promoter | blood | 9.94 |
| 181 | ENST00000483487.2 | SLC11A1 | 2 | 219248890 | promoter | blood | 5.23 |
| 182 | ENST00000483665.6 | FCGR2A | 1 | 161475247 | promoter | blood | 6.60 |
| 183 | ENST00000483722.1 | TREML2 | 6 | 41168932 | promoter | blood | 15.58 |
| 184 | ENST00000483750.5 | WAS | X | 48542217 | promoter | blood | 34.49 |
| 185 | ENST00000484216.1 | HBA2 | 16 | 222943 | promoter | blood | 296.56 |
| 186 | ENST00000484762.1 | CSF3R | 1 | 36933677 | promoter | blood | 8.26 |
| 187 | ENST00000485335.1 | IL1R2 | 2 | 102639852 | promoter | blood | 12.15 |
| 188 | ENST00000485341.5 | LSP1 | 11 | 1901907 | promoter | blood | 5.48 |
| 189 | ENST00000485583.1 | LRCH4 | 7 | 100183752 | promoter | blood | 5.95 |
| 190 | ENST00000485743.1 | HBB | 11 | 5248302 | promoter | blood | 128.52 |
| 191 | ENST00000485991.5 | CFP | X | 47489294 | promoter | blood | 5.10 |
| 192 | ENST00000486097.1 | NCF1 | 7 | 74197786 | promoter | blood | 12.51 |
| 193 | ENST00000486327.6 | DHX34 | 19 | 47870370 | promoter | blood | 6.88 |
| 194 | ENST00000486742.2 | LILRA5 | 19 | 54824344 | promoter | blood | 17.29 |
| 195 | ENST00000486749.5 | SLC2A3 | 12 | 8088763 | promoter | blood | 9.53 |
| 196 | ENST00000486935.2 | SEMA4D | 9 | 91996758 | promoter | blood | 5.98 |
| 197 | ENST00000487283.5 | ZAP70 | 2 | 98349392 | promoter | blood | 7.23 |
| 198 | ENST00000487290.5 | HCLS1 | 3 | 121379745 | promoter | blood | 6.41 |
| 199 | ENST00000487445.6 | GCA | 2 | 163200669 | promoter | blood | 12.82 |
| 200 | ENST00000487651.5 | SLC12A9 | 7 | 100456358 | promoter | blood | 6.67 |
| 201 | ENST00000487761.5 | GPSM3 | 6 | 32160645 | promoter | blood | 14.37 |
| 202 | ENST00000487791.1 | HBA1 | 16 | 226747 | promoter | blood | 29.54 |
| 203 | ENST00000487871.1 | SMAP2 | 1 | 40879649 | promoter | blood | 36.53 |
| 204 | ENST00000488945.5 | GNLY | 2 | 85912298 | promoter | blood | 11.68 |
| 205 | ENST00000488970.1 | BTK | X | 100609209 | promoter | blood | 7.40 |
| 206 | ENST00000489175.1 | KCNE1 | 21 | 35884505 | promoter | blood | 6.75 |
| 207 | ENST00000489358.5 | NAMPT | 7 | 105925396 | promoter | blood | 30.07 |
| 208 | ENST00000489551.5 | CSF3R | 1 | 36948535 | promoter | blood | 106.01 |
| 209 | ENST00000489980.5 | GNLY | 2 | 85921465 | promoter | blood | 14.53 |
| 210 | ENST00000490763.1 | SLC2A3 | 12 | 8082631 | promoter | blood | 5.93 |
| 211 | ENST00000490872.1 | SLC11A1 | 2 | 219254688 | promoter | blood | 26.15 |
| 212 | ENST00000491376.5 | IMPDH1 | 7 | 128050036 | promoter | blood | 6.59 |
| 213 | ENST00000492413.5 | SLC11A1 | 2 | 219247010 | promoter | blood | 17.89 |
| 214 | ENST00000493242.1 | LILRB2 | 19 | 54784952 | promoter | blood | 22.65 |
| 215 | ENST00000493288.1 | PGD | 1 | 10472891 | promoter | blood | 9.99 |
| 216 | ENST00000493365.5 | ABTB1 | 3 | 127391795 | promoter | blood | 6.22 |
| 217 | ENST00000493632.5 | INPP5D | 2 | 234078206 | promoter | blood | 17.01 |
| 218 | ENST00000493871.1 | MPP1 | X | 154014500 | promoter | blood | 6.09 |
| 219 | ENST00000494080.5 | CCDC88B | 11 | 64109078 | promoter | blood | 7.88 |
| 220 | ENST00000494268.1 | MSL3 | X | 11778341 | promoter | blood | 6.22 |
| 221 | ENST00000495406.1 | CDK5RAP2 | 9 | 123152555 | promoter | blood | 10.53 |
| 222 | ENST00000495458.5 | RAB24 | 5 | 176730682 | promoter | blood | 11.72 |
| 223 | ENST00000495988.1 | APOBEC3A | 22 | 39348746 | promoter | blood | 40.03 |
| 224 | ENST00000496379.5 | FES | 15 | 91433070 | promoter | blood | 7.77 |
| 225 | ENST00000496915.1 | PREX1 | 20 | 47259276 | promoter | blood | 5.23 |
| 226 | ENST00000497441.5 | RASGRP2 | 11 | 64504726 | promoter | blood | 12.78 |
| 227 | ENST00000497615.1 | NADK | 1 | 1686335 | promoter | blood | 9.43 |
| 228 | ENST00000497747.5 | NADK | 1 | 1686305 | promoter | blood | 9.33 |
| 229 | ENST00000498461.1 | ALOX5 | 10 | 45938869 | promoter | blood | 13.87 |
| 230 | ENST00000498666.5 | ITGB2 | 21 | 46340875 | promoter | blood | 13.12 |
| 231 | ENST00000500323.2 | DOK3 | 5 | 176935878 | promoter | blood | 10.88 |
| 232 | ENST00000502380.1 | DOK3 | 5 | 176937383 | promoter | blood | 8.44 |
| 233 | ENST00000504910.1 | HK3 | 5 | 176318392 | promoter | blood | 6.36 |
| 234 | ENST00000505207.5 | UBE2D3 | 4 | 103746937 | promoter | blood | 7.43 |
| 235 | ENST00000508794.6 | NAIP | 5 | 70316601 | promoter | blood | 11.43 |
| 236 | ENST00000509314.5 | FBXL5 | 4 | 15661487 | promoter | blood | 10.24 |
| 237 | ENST00000509339.1 | MXD3 | 5 | 176735063 | promoter | blood | 30.07 |
| 238 | ENST00000512426.5 | CCND3 | 6 | 41908323 | promoter | blood | 7.01 |
| 239 | ENST00000512747.3 | IL2RG | X | 70331456 | promoter | blood | 6.21 |
| 240 | ENST00000513001.5 | ACSL1 | 4 | 185680030 | promoter | blood | 17.65 |
| 241 | ENST00000517804.5 | DMTN | 8 | 21914477 | promoter | blood | 7.30 |
| 242 | ENST00000517813.1 | DENND3 | 8 | 142194587 | promoter | blood | 13.47 |
| 243 | ENST00000517985.5 | DENND3 | 8 | 142188220 | promoter | blood | 9.36 |
| 244 | ENST00000518135.1 | TNFRSF10C | 8 | 22972020 | promoter | blood | 5.06 |
| 245 | ENST00000518249.5 | DENND3 | 8 | 142151302 | promoter | blood | 9.51 |
| 246 | ENST00000518806.1 | DENND3 | 8 | 142187186 | promoter | blood | 11.76 |
| 247 | ENST00000519594.5 | LCP2 | 5 | 169724731 | promoter | blood | 5.37 |
| 248 | ENST00000520174.5 | DMTN | 8 | 21914416 | promoter | blood | 16.02 |
| 249 | ENST00000520322.1 | LCP2 | 5 | 169678696 | promoter | blood | 5.68 |
| 250 | ENST00000520450.5 | DOCK2 | 5 | 169483442 | promoter | blood | 11.23 |
| 251 | ENST00000520571.1 | DENND3 | 8 | 142198997 | promoter | blood | 8.57 |
| 252 | ENST00000520887.1 | FAM49B | 8 | 130859563 | promoter | blood | 7.15 |
| 253 | ENST00000521442.1 | ATP6V1B2 | 8 | 20075693 | promoter | blood | 8.33 |
| 254 | ENST00000523015.1 | DENND3 | 8 | 142154013 | promoter | blood | 7.05 |
| 255 | ENST00000523022.5 | CA1 | 8 | 86290342 | promoter | blood | 40.92 |
| 256 | ENST00000523066.1 | DENND3 | 8 | 142183604 | promoter | blood | 6.75 |
| 257 | ENST00000523266.5 | DMTN | 8 | 21916717 | promoter | blood | 6.93 |
| 258 | ENST00000523369.1 | LCP2 | 5 | 169685502 | promoter | blood | 9.88 |
| 259 | ENST00000523530.1 | DENND3 | 8 | 142201373 | promoter | blood | 7.23 |
| 260 | ENST00000523615.1 | STK10 | 5 | 171488634 | promoter | blood | 12.61 |
| 261 | ENST00000523829.5 | TMEM71 | 8 | 133772807 | promoter | blood | 11.05 |
| 262 | ENST00000526387.5 | TBC1D10C | 11 | 67171386 | promoter | blood | 7.10 |
| 263 | ENST00000526474.1 | TBC1D10C | 11 | 67172719 | promoter | blood | 8.87 |
| 264 | ENST00000526980.5 | CSF3R | 1 | 36948500 | promoter | blood | 184.80 |
| 265 | ENST00000527031.5 | JAK3 | 19 | 17958800 | promoter | blood | 6.17 |
| 266 | ENST00000527146.1 | IFITM2 | 11 | 308320 | promoter | blood | 18.56 |
| 267 | ENST00000527525.5 | SIPA1 | 11 | 65407337 | promoter | blood | 5.39 |
| 268 | ENST00000528086.5 | CD44 | 11 | 35160718 | promoter | blood | 8.17 |
| 269 | ENST00000528780.5 | IFITM1 | 11 | 313506 | promoter | blood | 47.88 |
| 270 | ENST00000530119.5 | TALDO1 | 11 | 747464 | promoter | blood | 7.74 |
| 271 | ENST00000530226.1 | SIPA1 | 11 | 65413282 | promoter | blood | 6.84 |
| 272 | ENST00000530587.5 | PHF21A | 11 | 45959815 | promoter | blood | 6.25 |
| 273 | ENST00000531221.5 | FCGR3B | 1 | 161600996 | promoter | blood | 57.18 |
| 274 | ENST00000531334.1 | ECE1 | 1 | 21548574 | promoter | blood | 5.78 |
| 275 | ENST00000532028.1 | PHF21A | 11 | 45967420 | promoter | blood | 5.50 |
| 276 | ENST00000532362.1 | CMTM2 | 16 | 66620812 | promoter | blood | 114.27 |
| 277 | ENST00000532897.5 | MKNK1 | 1 | 47037972 | promoter | blood | 6.23 |
| 278 | ENST00000533815.2 | BTNL8 | 5 | 180336564 | promoter | blood | 5.72 |
| 279 | ENST00000533968.1 | SPI1 | 11 | 47400038 | promoter | blood | 12.90 |
| 280 | ENST00000534261.3 | SIGLEC5 | 19 | 52148798 | promoter | blood | 6.14 |
| 281 | ENST00000534754.5 | SORL1 | 11 | 121481961 | promoter | blood | 9.84 |
| 282 | ENST00000535615.5 | CARS2 | 13 | 111318077 | promoter | blood | 5.33 |
| 283 | ENST00000535841.2 | DEFA1B | 8 | 6875811 | promoter | blood | 962.74 |
| 284 | ENST00000536885.1 | ARAP1 | 11 | 72399526 | promoter | blood | 7.74 |
| 285 | ENST00000536995.5 | ATG16L2 | 11 | 72532933 | promoter | blood | 11.55 |
| 286 | ENST00000538842.1 | ATG16L2 | 11 | 72527790 | promoter | blood | 6.35 |
| 287 | ENST00000539134.1 | RELT | 11 | 73104289 | promoter | blood | 6.58 |
| 288 | ENST00000539932.5 | SLC11A1 | 2 | 219246926 | promoter | blood | 50.32 |
| 289 | ENST00000540182.5 | NLRC5 | 16 | 57074600 | promoter | blood | 6.20 |
| 290 | ENST00000540513.1 | ACRBP | 12 | 6748017 | promoter | blood | 9.23 |
| 291 | ENST00000541206.6 | RNF166 | 16 | 88770025 | promoter | blood | 11.36 |
| 292 | ENST00000541623.5 | ARAP1 | 11 | 72399582 | promoter | blood | 8.01 |
| 293 | ENST00000541800.6 | PLBD1 | 12 | 14689059 | promoter | blood | 6.78 |
| 294 | ENST00000542481.1 | ATG16L2 | 11 | 72534940 | promoter | blood | 25.18 |
| 295 | ENST00000542587.5 | LPAR2 | 19 | 19739739 | promoter | blood | 10.95 |
| 296 | ENST00000543576.5 | DENND1C | 19 | 6481798 | promoter | blood | 12.06 |
| 297 | ENST00000544490.5 | ATG16L2 | 11 | 72525472 | promoter | blood | 30.13 |
| 298 | ENST00000544997.5 | FERMT3 | 11 | 63974150 | promoter | blood | 7.56 |
| 299 | ENST00000546200.5 | ARHGAP9 | 12 | 57871634 | promoter | blood | 66.03 |
| 300 | ENST00000546704.1 | ARHGAP9 | 12 | 57868923 | promoter | blood | 19.20 |
| 301 | ENST00000548139.5 | ARHGAP9 | 12 | 57871842 | promoter | blood | 15.20 |
| 302 | ENST00000551000.1 | ARHGAP9 | 12 | 57871650 | promoter | blood | 8.28 |
| 303 | ENST00000552953.5 | ARHGAP9 | 12 | 57870424 | promoter | blood | 24.58 |
| 304 | ENST00000553070.5 | NFE2 | 12 | 54694799 | promoter | blood | 23.61 |
| 305 | ENST00000553872.1 | PYGL | 14 | 51388246 | promoter | blood | 8.64 |
| 306 | ENST00000554736.5 | GNG2 | 14 | 52328042 | promoter | blood | 9.30 |
| 307 | ENST00000556418.1 | RIN3 | 14 | 93118846 | promoter | blood | 12.65 |
| 308 | ENST00000558177.5 | ANPEP | 15 | 90345967 | promoter | blood | 11.96 |
| 309 | ENST00000558332.3 | IL16 | 15 | 81591757 | promoter | blood | 7.66 |
| 310 | ENST00000558588.5 | PLCB2 | 15 | 40599894 | promoter | blood | 12.82 |
| 311 | ENST00000559341.5 | MAN2A2 | 15 | 91459225 | promoter | blood | 8.61 |
| 312 | ENST00000559750.5 | PSTPIP1 | 15 | 77287513 | promoter | blood | 16.39 |
| 313 | ENST00000560064.1 | PSTPIP1 | 15 | 77328035 | promoter | blood | 5.96 |
| 314 | ENST00000560377.5 | PSTPIP1 | 15 | 77287726 | promoter | blood | 8.38 |
| 315 | ENST00000561175.1 | ADAM8 | 10 | 135086080 | promoter | blood | 6.86 |
| 316 | ENST00000562129.1 | CORO1A | 16 | 30199396 | promoter | blood | 14.38 |
| 317 | ENST00000562759.1 | ST20 | 15 | 80200378 | promoter | blood | 5.59 |
| 318 | ENST00000562918.5 | ITGAX | 16 | 31366495 | promoter | blood | 10.15 |
| 319 | ENST00000563269.1 | PLCG2 | 16 | 81964866 | promoter | blood | 6.38 |
| 320 | ENST00000563526.5 | CYBA | 16 | 88717396 | promoter | blood | 7.13 |
| 321 | ENST00000564072.1 | SLCO3A1 | 15 | 92704805 | promoter | blood | 8.74 |
| 322 | ENST00000564572.1 | CNN2 | 19 | 1036798 | promoter | blood | 5.34 |
| 323 | ENST00000564632.1 | ITGAL | 16 | 30531077 | promoter | blood | 12.98 |
| 324 | ENST00000564707.1 | AHSP | 16 | 31539298 | promoter | blood | 6.85 |
| 325 | ENST00000564768.1 | CORO1A | 16 | 30198003 | promoter | blood | 6.01 |
| 326 | ENST00000564894.1 | RNF166 | 16 | 88768058 | promoter | blood | 7.90 |
| 327 | ENST00000564905.1 | XPO6 | 16 | 28145246 | promoter | blood | 12.98 |
| 328 | ENST00000565076.1 | SIRPB1 | 20 | 1552558 | promoter | blood | 5.74 |
| 329 | ENST00000565284.5 | XPO6 | 16 | 28164239 | promoter | blood | 14.84 |
| 330 | ENST00000565497.5 | CORO1A | 16 | 30194940 | promoter | blood | 6.42 |
| 331 | ENST00000566082.1 | USB1 | 16 | 58049017 | promoter | blood | 6.82 |
| 332 | ENST00000567034.5 | CORO1A | 16 | 30197253 | promoter | blood | 11.54 |
| 333 | ENST00000567038.1 | XPO6 | 16 | 28113307 | promoter | blood | 8.97 |
| 334 | ENST00000567178.1 | ITGAM | 16 | 31339358 | promoter | blood | 23.09 |
| 335 | ENST00000568760.5 | DEF8 | 16 | 90023661 | promoter | blood | 7.24 |
| 336 | ENST00000568982.5 | CORO1A | 16 | 30194930 | promoter | blood | 7.54 |
| 337 | ENST00000570106.6 | SIGLEC5 | 19 | 52133588 | promoter | blood | 40.53 |
| 338 | ENST00000570439.1 | ACAP1 | 17 | 7248650 | promoter | blood | 5.27 |
| 339 | ENST00000570473.5 | TRIM25 | 17 | 54981704 | promoter | blood | 5.32 |
| 340 | ENST00000570755.1 | MMP25 | 16 | 3097532 | promoter | blood | 31.89 |
| 341 | ENST00000571206.1 | ARRB2 | 17 | 4618884 | promoter | blood | 26.58 |
| 342 | ENST00000571220.1 | ACAP1 | 17 | 7246254 | promoter | blood | 29.18 |
| 343 | ENST00000571303.1 | MLKL | 16 | 74725367 | promoter | blood | 5.93 |
| 344 | ENST00000571791.5 | ARRB2 | 17 | 4613977 | promoter | blood | 13.94 |
| 345 | ENST00000572418.1 | P2RX1 | 17 | 3819746 | promoter | blood | 9.95 |
| 346 | ENST00000572666.5 | CORO7 | 16 | 4411903 | promoter | blood | 7.67 |
| 347 | ENST00000572782.1 | ARRB2 | 17 | 4617846 | promoter | blood | 16.63 |
| 348 | ENST00000574548.1 | RNF167 | 17 | 4847675 | promoter | blood | 6.40 |
| 349 | ENST00000574888.5 | ARRB2 | 17 | 4613950 | promoter | blood | 7.24 |
| 350 | ENST00000575131.5 | ARRB2 | 17 | 4613956 | promoter | blood | 11.97 |
| 351 | ENST00000575441.1 | MMP25 | 16 | 3106795 | promoter | blood | 88.59 |
| 352 | ENST00000576594.1 | ACAP1 | 17 | 7250448 | promoter | blood | 17.88 |
| 353 | ENST00000576628.1 | ACAP1 | 17 | 7239916 | promoter | blood | 20.20 |
| 354 | ENST00000576769.1 | SLC43A2 | 17 | 1486992 | promoter | blood | 5.40 |
| 355 | ENST00000578574.1 | SLC16A3 | 17 | 80186938 | promoter | blood | 7.93 |
| 356 | ENST00000581287.5 | SLC16A3 | 17 | 80191563 | promoter | blood | 11.97 |
| 357 | ENST00000581974.1 | DHRS13 | 17 | 27226541 | promoter | blood | 5.97 |
| 358 | ENST00000582174.5 | FLOT2 | 17 | 27224672 | promoter | blood | 6.46 |
| 359 | ENST00000582812.5 | NDEL1 | 17 | 8316449 | promoter | blood | 9.15 |
| 360 | ENST00000583093.5 | SECTM1 | 17 | 80291639 | promoter | blood | 7.78 |
| 361 | ENST00000583810.5 | PIK3R5 | 17 | 8785825 | promoter | blood | 8.43 |
| 362 | ENST00000584445.5 | NARF | 17 | 80438344 | promoter | blood | 11.89 |
| 363 | ENST00000585852.5 | FMNL1 | 17 | 43308036 | promoter | blood | 19.81 |
| 364 | ENST00000586080.1 | R3HDM4 | 19 | 901236 | promoter | blood | 5.15 |
| 365 | ENST00000587259.5 | VMP1 | 17 | 57807076 | promoter | blood | 10.58 |
| 366 | ENST00000587265.1 | CA4 | 17 | 58235469 | promoter | blood | 5.45 |
| 367 | ENST00000587287.1 | RASGRP4 | 19 | 38902036 | promoter | blood | 6.80 |
| 368 | ENST00000587444.1 | VASP | 19 | 46029052 | promoter | blood | 6.41 |
| 369 | ENST00000587856.1 | FMNL1 | 17 | 43311251 | promoter | blood | 22.20 |
| 370 | ENST00000588708.5 | RASGRP4 | 19 | 38907787 | promoter | blood | 24.68 |
| 371 | ENST00000589261.5 | ICAM3 | 19 | 10450295 | promoter | blood | 6.41 |
| 372 | ENST00000589900.5 | ICAM3 | 19 | 10450305 | promoter | blood | 21.24 |
| 373 | ENST00000589911.1 | FMNL1 | 17 | 43322691 | promoter | blood | 7.99 |
| 374 | ENST00000590227.5 | DNAH17 | 17 | 76446380 | promoter | blood | 5.85 |
| 375 | ENST00000590856.1 | UNC13D | 17 | 73826742 | promoter | blood | 6.33 |
| 376 | ENST00000590974.1 | LYL1 | 19 | 13213975 | promoter | blood | 5.48 |
| 377 | ENST00000591616.1 | UNC13D | 17 | 73831353 | promoter | blood | 7.12 |
| 378 | ENST00000592527.1 | FMNL1 | 17 | 43310229 | promoter | blood | 7.31 |
| 379 | ENST00000594221.5 | STXBP2 | 19 | 7706716 | promoter | blood | 6.21 |
| 380 | ENST00000594696.1 | PRAM1 | 19 | 8556907 | promoter | blood | 17.63 |
| 381 | ENST00000594743.1 | CD37 | 19 | 49840472 | promoter | blood | 8.07 |
| 382 | ENST00000595046.1 | MYO1F | 19 | 8642326 | promoter | blood | 6.90 |
| 383 | ENST00000595325.5 | MYO1F | 19 | 8642322 | promoter | blood | 13.66 |
| 384 | ENST00000595596.1 | GRAMD1A | 19 | 35503892 | promoter | blood | 5.53 |
| 385 | ENST00000595725.5 | CD37 | 19 | 49838657 | promoter | blood | 20.04 |
| 386 | ENST00000596937.1 | MYO1F | 19 | 8615297 | promoter | blood | 6.21 |
| 387 | ENST00000597222.1 | MYO1F | 19 | 8604936 | promoter | blood | 29.52 |
| 388 | ENST00000597611.7 | FKBP8 | 19 | 18654887 | promoter | blood | 9.25 |
| 389 | ENST00000597852.5 | CD37 | 19 | 49838675 | promoter | blood | 13.98 |
| 390 | ENST00000598005.1 | MYO1F | 19 | 8606840 | promoter | blood | 17.89 |
| 391 | ENST00000598201.5 | SHKBP1 | 19 | 41086073 | promoter | blood | 12.36 |
| 392 | ENST00000598529.5 | MYO1F | 19 | 8619448 | promoter | blood | 16.08 |
| 393 | ENST00000598907.5 | SHKBP1 | 19 | 41084102 | promoter | blood | 14.71 |
| 394 | ENST00000598953.1 | FPR2 | 19 | 52264465 | promoter | blood | 37.43 |
| 395 | ENST00000599123.1 | MYO1F | 19 | 8620661 | promoter | blood | 7.54 |
| 396 | ENST00000599180.2 | FFAR2 | 19 | 35939203 | promoter | blood | 24.79 |
| 397 | ENST00000599662.1 | CEACAM3 | 19 | 42312851 | promoter | blood | 9.48 |
| 398 | ENST00000599694.1 | RASAL3 | 19 | 15567410 | promoter | blood | 6.37 |
| 399 | ENST00000599716.5 | SHKBP1 | 19 | 41082793 | promoter | blood | 10.66 |
| 400 | ENST00000600463.1 | IFI30 | 19 | 18284979 | promoter | blood | 6.41 |
| 401 | ENST00000600885.1 | MYO1F | 19 | 8592271 | promoter | blood | 5.95 |
| 402 | ENST00000601502.1 | MYO1F | 19 | 8610602 | promoter | blood | 8.05 |
| 403 | ENST00000602101.6 | RASAL3 | 19 | 15575325 | promoter | blood | 7.16 |
| 404 | ENST00000602136.1 | MYO1F | 19 | 8612931 | promoter | blood | 7.01 |
| 405 | ENST00000602142.5 | VAV1 | 19 | 6772737 | promoter | blood | 8.46 |
| 406 | ENST00000602166.1 | EGLN2 | 19 | 41312824 | promoter | blood | 5.19 |
| 407 | ENST00000602612.5 | KCNAB2 | 1 | 6086380 | promoter | blood | 5.03 |
| 408 | ENST00000605039.5 | BIN2 | 12 | 51717938 | promoter | blood | 11.02 |
| 409 | ENST00000607855.5 | CD177 | 19 | 43857843 | promoter | blood | 5.94 |
| 410 | ENST00000609870.5 | CELF2 | 10 | 11207447 | promoter | blood | 12.41 |
| 411 | ENST00000611028.2 | FOLR3 | 11 | 71846798 | promoter | blood | 11.13 |
| 412 | ENST00000612844.4 | FOLR3 | 11 | 71846756 | promoter | blood | 13.11 |
| 413 | ENST00000614135.4 | RASGRP4 | 19 | 38916945 | promoter | blood | 8.18 |
| 414 | ENST00000614254.1 | AOAH | 7 | 36570290 | promoter | blood | 14.33 |
| 415 | ENST00000615340.4 | RASGRP4 | 19 | 38916837 | promoter | blood | 6.88 |
| 416 | ENST00000616356.4 | FCN1 | 9 | 137809723 | promoter | blood | 16.99 |
| 417 | ENST00000619100.4 | SLC38A5 | X | 48325880 | promoter | blood | 10.86 |
| 418 | ENST00000620888.4 | HBG2 | 11 | 5276011 | promoter | blood | 161.14 |
| 419 | ENST00000621510.1 | AOAH | 7 | 36554282 | promoter | blood | 19.27 |
| 420 | ENST00000622764.1 | SAP25 | 7 | 100171443 | promoter | blood | 5.94 |
| 421 | ENST00000008938.4 | PGLYRP1 | 19 | 46525993 | junction | blood | 148.76 |
| 422 | ENST00000177694.1 | TBX21 | 17 | 45811311 | junction | blood | 5.98 |
| 423 | ENST00000194097.8 | NAIP | 5 | 70316486 | junction | blood | 10.22 |
| 424 | ENST00000199708.2 | HBQ1 | 15 | 230580 | junction | blood | 45.79 |
| 425 | ENST00000217133.1 | TUBB1 | 20 | 57594634 | junction | blood | 6.01 |
| 426 | ENST00000219596.5 | MEFV | 16 | 3306311 | junction | blood | 19.19 |
| 427 | ENST00000221804.4 | CLC | 19 | 40228578 | junction | blood | 19.65 |
| 428 | ENST00000221954.6 | CEACAM4 | 19 | 42133268 | junction | blood | 32.81 |
| 429 | ENST00000225275.3 | MPO | 17 | 56357966 | junction | blood | 7.23 |
| 430 | ENST00000233997.3 | AZU1 | 19 | 827904 | junction | blood | 14.03 |
| 431 | ENST00000234347.9 | PRTN3 | 19 | 841069 | junction | blood | 13.78 |
| 432 | ENST00000236826.7 | MMP8 | 11 | 102595485 | junction | blood | 20.68 |
| 433 | ENST00000245620.13 | LILRB3 | 19 | 54726815 | junction | blood | 22.83 |
| 434 | ENST00000258104.7 | DYSF | 2 | 71681216 | junction | blood | 6.76 |
| 435 | ENST00000262407.5 | ITGA2B | 17 | 42466654 | junction | blood | 12.34 |
| 436 | ENST00000262651.3 | HCK | 20 | 30640289 | junction | blood | 8.74 |
| 437 | ENST00000262865.8 | BPI | 20 | 36932755 | junction | blood | 18.06 |
| 438 | ENST00000263621.1 | ELANE | 19 | 852395 | junction | blood | 21.67 |
| 439 | ENST00000264260.6 | IL18RAP | 2 | 103035400 | junction | blood | 42.11 |
| 440 | ENST00000264834.4 | KLF1 | 19 | 12997868 | junction | blood | 8.95 |
| 441 | ENST00000267396.8 | REM2 | 14 | 23352599 | junction | blood | 6.91 |
| 442 | ENST00000287497.12 | ITGAM | 16 | 31271413 | junction | blood | 6.52 |
| 443 | ENST00000294800.7 | FCGR3B | 1 | 161600845 | junction | blood | 13.85 |
| 444 | ENST00000295619.3 | PROK2 | 3 | 71834108 | junction | blood | 12.98 |
| 445 | ENST00000295683.2 | CXCR1 | 2 | 219031631 | junction | blood | 355.57 |
| 446 | ENST00000296028.3 | PPBP | 4 | 74853673 | junction | blood | 50.15 |
| 447 | ENST00000296435.2 | CAMP | 3 | 48265202 | junction | blood | 27.17 |
| 448 | ENST00000297435.2 | DEFA4 | 8 | 6795748 | junction | blood | 34.98 |
| 449 | ENST00000299663.7 | CLEC4E | 12 | 8693357 | junction | blood | 29.27 |
| 450 | ENST00000299665.2 | CLEC4D | 12 | 8666356 | junction | blood | 10.47 |
| 451 | ENST00000302312.8 | AHSP | 16 | 31539283 | junction | blood | 85.51 |
| 452 | ENST00000304076.6 | VAV1 | 19 | 6773022 | junction | blood | 7.61 |
| 453 | ENST00000304361.8 | CLEC12A | 12 | 10124286 | junction | blood | 10.23 |
| 454 | ENST00000307395.4 | GP9 | 3 | 128779693 | junction | blood | 9.72 |
| 455 | ENST00000307564.8 | AKNA | 9 | 117156637 | junction | blood | 11.95 |
| 456 | ENST00000310544.8 | PHOSPHO1 | 17 | 47307830 | junction | blood | 30.92 |
| 457 | ENST00000312156.8 | NFE2 | 12 | 54689328 | junction | blood | 11.15 |
| 458 | ENST00000314412.6 | FUT7 | 9 | 139926431 | junction | blood | 16.90 |
| 459 | ENST00000314446.9 | LILRB2 | 19 | 54784816 | junction | blood | 7.87 |
| 460 | ENST00000318507.6 | CXCR2 | 2 | 218991076 | junction | blood | 157.27 |
| 461 | ENST00000324134.10 | NLRP12 | 19 | 54327140 | junction | blood | 9.17 |
| 462 | ENST00000326965.6 | TAGAP | 6 | 159465911 | junction | blood | 5.94 |
| 463 | ENST00000327857.6 | DEFA3 | 8 | 6875744 | junction | blood | 355.10 |
| 464 | ENST00000329800.7 | TMCC2 | 1 | 205225544 | junction | blood | 10.64 |
| 465 | ENST00000330597.3 | HBG1 | 11 | 5270943 | junction | blood | 5.07 |
| 466 | ENST00000330807.9 | ALAS2 | X | 55057375 | junction | blood | 142.66 |
| 467 | ENST00000331941.6 | CSF3R | 1 | 36945034 | junction | blood | 16.12 |
| 468 | ENST00000336906.4 | HBG2 | 11 | 5275867 | junction | blood | 104.85 |
| 469 | ENST00000338372.6 | VSTM1 | 19 | 54566998 | junction | blood | 10.71 |
| 470 | ENST00000340023.6 | FPR2 | 19 | 52264483 | junction | blood | 34.71 |
| 471 | ENST00000342063.4 | C19orf35 | 19 | 2282086 | junction | blood | 8.64 |
| 472 | ENST00000342571.7 | MKNK1 | 1 | 47051546 | junction | blood | 14.42 |
| 473 | ENST00000344138.8 | GRAP2 | 22 | 40297334 | junction | blood | 5.60 |
| 474 | ENST00000348343.10 | BMX | X | 15519063 | junction | blood | 6.99 |
| 475 | ENST00000355524.7 | FCAR | 19 | 55385779 | junction | blood | 53.04 |
| 476 | ENST00000356815.3 | HBM | 16 | 216088 | junction | blood | 163.82 |
| 477 | ENST00000357396.7 | CEACAM3 | 19 | 42300673 | junction | blood | 16.26 |
| 478 | ENST00000367053.5 | CR1 | 1 | 207669733 | junction | blood | 10.47 |
| 479 | ENST00000367964.6 | FCGR3B | 1 | 161601676 | junction | blood | 465.71 |
| 480 | ENST00000368034.8 | CD244 | 1 | 160832407 | junction | blood | 6.27 |
| 481 | ENST00000373103.5 | CSF3R | 1 | 36948412 | junction | blood | 61.20 |
| 482 | ENST00000375448.4 | PADI4 | 1 | 17634809 | junction | blood | 95.04 |
| 483 | ENST00000375809.7 | C6orf25 | 6 | 31691221 | junction | blood | 5.36 |
| 484 | ENST00000376092.7 | LST1 | 6 | 31555095 | junction | blood | 5.01 |
| 485 | ENST00000376670.7 | GATA1 | X | 48645053 | junction | blood | 11.17 |
| 486 | ENST00000377901.8 | TMEM71 | 8 | 133772722 | junction | blood | 6.74 |
| 487 | ENST00000378023.8 | FAM65B | 6 | 24877179 | junction | blood | 15.67 |
| 488 | ENST00000378625.5 | NADK | 1 | 1711344 | junction | blood | 13.09 |
| 489 | ENST00000379595.7 | PSTPIP1 | 15 | 77287950 | junction | blood | 5.90 |
| 490 | ENST00000380299.3 | HBD | 11 | 5255572 | junction | blood | 138.84 |
| 491 | ENST00000380315.2 | HBB | 11 | 5250482 | junction | blood | 5.45 |
| 492 | ENST00000381153.7 | C11orf21 | 11 | 2322986 | junction | blood | 11.33 |
| 493 | ENST00000381603.7 | SIRPB1 | 20 | 1600515 | junction | blood | 26.01 |
| 494 | ENST00000382689.7 | DEFA1B | 8 | 6856645 | junction | blood | 118.00 |
| 495 | ENST00000382692.2 | DEFA1 | 8 | 6837535 | junction | blood | 996.76 |
| 496 | ENST00000392619.1 | CD300E | 17 | 72619721 | junction | blood | 24.98 |
| 497 | ENST00000392621.5 | CD300LB | 17 | 72527450 | junction | blood | 7.72 |
| 498 | ENST00000392841.1 | HMBS | 11 | 118958788 | junction | blood | 7.28 |
| 499 | ENST00000393450.5 | MYL4 | 17 | 45286923 | junction | blood | 22.77 |
| 500 | ENST00000394430.5 | RASGRP2 | 11 | 64510266 | junction | blood | 17.61 |
| 501 | ENST00000394432.7 | RASGRP2 | 11 | 64511475 | junction | blood | 9.91 |
| 502 | ENST00000397497.8 | PTK2B | 8 | 27288989 | junction | blood | 5.81 |
| 503 | ENST00000397797.1 | HBA1 | 16 | 226761 | junction | blood | 25.27 |
| 504 | ENST00000398421.6 | NCF1 | 7 | 74188450 | junction | blood | 11.04 |
| 505 | ENST00000398632.3 | MX2 | 21 | 42775563 | junction | blood | 7.28 |
| 506 | ENST00000399753.2 | MSRB1 | 16 | 1993103 | junction | blood | 11.22 |
| 507 | ENST00000402255.5 | APOBEC3A | 22 | 39348855 | junction | blood | 11.96 |
| 508 | ENST00000402280.5 | NLRC4 | 2 | 32489807 | junction | blood | 5.84 |
| 509 | ENST00000404054.3 | TACC3 | 4 | 1743848 | junction | blood | 5.17 |
| 510 | ENST00000409369.1 | IL18RAP | 2 | 103036257 | junction | blood | 46.86 |
| 511 | ENST00000410026.6 | NABP1 | 2 | 192542967 | junction | blood | 7.35 |
| 512 | ENST00000414723.1 | GCA | 2 | 163212991 | junction | blood | 9.14 |
| 513 | ENST00000418507.6 | LST1 | 6 | 31554092 | junction | blood | 6.17 |
| 514 | ENST00000425417.1 | ARHGAP26 | 5 | 142586873 | junction | blood | 5.74 |
| 515 | ENST00000425515.6 | VNN3 | 6 | 133055619 | junction | blood | 8.73 |
| 516 | ENST00000426897.6 | CD37 | 19 | 49838832 | junction | blood | 7.43 |
| 517 | ENST00000427321.1 | C1orf228 | 1 | 45189578 | junction | blood | 8.41 |
| 518 | ENST00000427466.1 | THEMIS2 | 1 | 28199176 | junction | blood | 7.94 |
| 519 | ENST00000430041.6 | ARHGAP9 | 12 | 57871568 | junction | blood | 22.12 |
| 520 | ENST00000436504.2 | LILRB3 | 19 | 54722998 | junction | blood | 69.95 |
| 521 | ENST00000436693.6 | TLR6 | 4 | 38858382 | junction | blood | 5.83 |
| 522 | ENST00000441002.1 | IL1R2 | 2 | 102625104 | junction | blood | 16.30 |
| 523 | ENST00000441045.5 | NAMPT | 7 | 105925274 | junction | blood | 5.42 |
| 524 | ENST00000441366.6 | EPB42 | 15 | 43513014 | junction | blood | 11.69 |
| 525 | ENST00000442948.3 | FOLR3 | 11 | 71847166 | junction | blood | 9.64 |
| 526 | ENST00000444257.5 | PLB1 | 2 | 28827626 | junction | blood | 7.51 |
| 527 | ENST00000448367.5 | FES | 15 | 91432866 | junction | blood | 6.30 |
| 528 | ENST00000452208.1 | RGL4 | 22 | 24038875 | junction | blood | 64.94 |
| 529 | ENST00000453777.1 | LILRA1 | 19 | 55105246 | junction | blood | 6.64 |
| 530 | ENST00000455062.2 | NCF1 | 7 | 74191693 | junction | blood | 15.11 |
| 531 | ENST00000456013.5 | PTPN6 | 12 | 7060683 | junction | blood | 7.47 |
| 532 | ENST00000460045.1 | IMPDH1 | 7 | 128035185 | junction | blood | 8.72 |
| 533 | ENST00000460208.1 | LILRB3 | 19 | 54721188 | junction | blood | 51.15 |
| 534 | ENST00000460442.5 | ZDHHC19 | 3 | 195925660 | junction | blood | 22.43 |
| 535 | ENST00000460650.5 | SELL | 1 | 169673709 | junction | blood | 5.03 |
| 536 | ENST00000462275.5 | TBXAS1 | 7 | 139529278 | junction | blood | 6.07 |
| 537 | ENST00000462927.5 | CYTH4 | 22 | 37678629 | junction | blood | 26.95 |
| 538 | ENST00000463153.1 | ABHD5 | 3 | 43759349 | junction | blood | 5.85 |
| 539 | ENST00000463516.5 | MYO1G | 7 | 45014773 | junction | blood | 5.23 |
| 540 | ENST00000464329.1 | CD53 | 1 | 111440514 | junction | blood | 10.28 |
| 541 | ENST00000464591.1 | C1orf162 | 1 | 112018687 | junction | blood | 7.62 |
| 542 | ENST00000464994.5 | IL1R2 | 2 | 102608495 | junction | blood | 12.24 |
| 543 | ENST00000465814.5 | ARAP1 | 11 | 72432895 | junction | blood | 10.09 |
| 544 | ENST00000465984.5 | SLC11A1 | 2 | 219247098 | junction | blood | 10.54 |
| 545 | ENST00000466138.1 | CSF3R | 1 | 36933159 | junction | blood | 32.37 |
| 546 | ENST00000466151.1 | PADI2 | 1 | 17409731 | junction | blood | 7.13 |
| 547 | ENST00000466612.5 | ABTB1 | 3 | 127392451 | junction | blood | 6.18 |
| 548 | ENST00000467001.1 | PADI4 | 1 | 17675280 | junction | blood | 24.03 |
| 549 | ENST00000467525.5 | FCGR2A | 1 | 161476381 | junction | blood | 8.40 |
| 550 | ENST00000467786.1 | FGD3 | 9 | 95737687 | junction | blood | 43.15 |
| 551 | ENST00000467972.5 | SLC12A9 | 7 | 100456564 | junction | blood | 6.80 |
| 552 | ENST00000468945.1 | PADI4 | 1 | 17672634 | junction | blood | 17.46 |
| 553 | ENST00000469273.1 | LILRB3 | 19 | 54724398 | junction | blood | 16.30 |
| 554 | ENST00000469280.1 | NCF2 | 1 | 183532569 | junction | blood | 13.91 |
| 555 | ENST00000469349.1 | NBEAL2 | 3 | 47049676 | junction | blood | 7.54 |
| 556 | ENST00000469380.1 | CSF3R | 1 | 36939365 | junction | blood | 5.77 |
| 557 | ENST00000469449.1 | SLC11A1 | 2 | 219249088 | junction | blood | 5.27 |
| 558 | ENST00000469532.1 | RAC2 | 22 | 37640154 | junction | blood | 9.83 |
| 559 | ENST00000470579.5 | ARID5A | 2 | 97202567 | junction | blood | 7.93 |
| 560 | ENST00000471466.5 | RAB24 | 5 | 176730010 | junction | blood | 6.58 |
| 561 | ENST00000471836.1 | PARVG | 22 | 44577797 | junction | blood | 6.53 |
| 562 | ENST00000472694.1 | HBA1 | 16 | 227132 | junction | blood | 64.74 |
| 563 | ENST00000473070.1 | FAM65B | 6 | 24843090 | junction | blood | 19.03 |
| 564 | ENST00000473883.5 | HCLS1 | 3 | 121361774 | junction | blood | 36.16 |
| 565 | ENST00000474129.1 | ABTB1 | 3 | 127395926 | junction | blood | 8.59 |
| 566 | ENST00000475472.5 | FGR | 1 | 27961576 | junction | blood | 49.95 |
| 567 | ENST00000475683.1 | TUBA4A | 2 | 220118578 | junction | blood | 10.51 |
| 568 | ENST00000476050.5 | TRAF3IP3 | 1 | 209942005 | junction | blood | 5.61 |
| 569 | ENST00000476408.1 | CD53 | 1 | 111422954 | junction | blood | 10.54 |
| 570 | ENST00000476492.1 | LAPTM5 | 1 | 31230506 | junction | blood | 6.02 |
| 571 | ENST00000476532.1 | SASH3 | X | 128914130 | junction | blood | 9.72 |
| 572 | ENST00000477720.1 | LILRA5 | 19 | 54822684 | junction | blood | 16.70 |
| 573 | ENST00000478298.5 | ABTB1 | 3 | 127395424 | junction | blood | 6.71 |
| 574 | ENST00000478902.1 | ZDHHC18 | 1 | 27177722 | junction | blood | 12.52 |
| 575 | ENST00000479534.5 | PADI2 | 1 | 17401351 | junction | blood | 5.78 |
| 576 | ENST00000479996.1 | RGS19 | 20 | 62710647 | junction | blood | 9.02 |
| 577 | ENST00000480467.5 | CDK5RAP2 | 9 | 123165521 | junction | blood | 16.88 |
| 578 | ENST00000480825.6 | CSF3R | 1 | 36940978 | junction | blood | 127.36 |
| 579 | ENST00000481215.1 | RAC2 | 22 | 37627271 | junction | blood | 7.04 |
| 580 | ENST00000481314.5 | HCLS1 | 3 | 121379667 | junction | blood | 13.32 |
| 581 | ENST00000481517.1 | STAT5B | 17 | 40369395 | junction | blood | 8.48 |
| 582 | ENST00000481789.1 | KCNAB2 | 1 | 6156816 | junction | blood | 8.66 |
| 583 | ENST00000482429.1 | LTB | 6 | 31549336 | junction | blood | 5.85 |
| 584 | ENST00000482543.1 | PTK2B | 8 | 27310677 | junction | blood | 9.94 |
| 585 | ENST00000483665.6 | FCGR2A | 1 | 161475342 | junction | blood | 6.60 |
| 586 | ENST00000483722.1 | TREML2 | 6 | 41168692 | junction | blood | 15.58 |
| 587 | ENST00000483750.5 | WAS | X | 48542374 | junction | blood | 34.49 |
| 588 | ENST00000484216.1 | HBA2 | 16 | 223006 | junction | blood | 296.56 |
| 589 | ENST00000484762.1 | CSF3R | 1 | 36933423 | junction | blood | 8.26 |
| 590 | ENST00000485335.1 | IL1R2 | 2 | 102640083 | junction | blood | 12.15 |
| 591 | ENST00000485341.5 | LSP1 | 11 | 1902593 | junction | blood | 5.48 |
| 592 | ENST00000485583.1 | LRCH4 | 7 | 100183504 | junction | blood | 5.95 |
| 593 | ENST00000485743.1 | HBB | 11 | 5248160 | junction | blood | 128.52 |
| 594 | ENST00000485991.5 | CFP | X | 47489168 | junction | blood | 5.10 |
| 595 | ENST00000486097.1 | NCF1 | 7 | 74197975 | junction | blood | 12.51 |
| 596 | ENST00000486327.6 | DHX34 | 19 | 47870412 | junction | blood | 6.88 |
| 597 | ENST00000486742.2 | LILRA5 | 19 | 54824287 | junction | blood | 17.29 |
| 598 | ENST00000486749.5 | SLC2A3 | 12 | 8088616 | junction | blood | 9.53 |
| 599 | ENST00000486935.2 | SEMA4D | 9 | 91996089 | junction | blood | 5.98 |
| 600 | ENST00000487283.5 | ZAP70 | 2 | 98349484 | junction | blood | 7.23 |
| 601 | ENST00000487445.6 | GCA | 2 | 163200785 | junction | blood | 12.82 |
| 602 | ENST00000487651.5 | SLC12A9 | 7 | 100459206 | junction | blood | 6.67 |
| 603 | ENST00000487761.5 | GPSM3 | 6 | 32160249 | junction | blood | 14.37 |
| 604 | ENST00000487791.1 | HBA1 | 16 | 226810 | junction | blood | 29.54 |
| 605 | ENST00000487871.1 | SMAP2 | 1 | 40879912 | junction | blood | 36.53 |
| 606 | ENST00000488945.5 | GNLY | 2 | 85912344 | junction | blood | 11.68 |
| 607 | ENST00000488970.1 | BTK | X | 100608858 | junction | blood | 7.40 |
| 608 | ENST00000489175.1 | KCNE1 | 21 | 35884325 | junction | blood | 6.75 |
| 609 | ENST00000489980.5 | GNLY | 2 | 85922546 | junction | blood | 14.53 |
| 610 | ENST00000490763.1 | SLC2A3 | 12 | 8082280 | junction | blood | 5.93 |
| 611 | ENST00000490872.1 | SLC11A1 | 2 | 219254751 | junction | blood | 26.15 |
| 612 | ENST00000491376.5 | IMPDH1 | 7 | 128049810 | junction | blood | 6.59 |
| 613 | ENST00000493288.1 | PGD | 1 | 10473308 | junction | blood | 9.99 |
| 614 | ENST00000493365.5 | ABTB1 | 3 | 127391924 | junction | blood | 6.22 |
| 615 | ENST00000493632.5 | INPP5D | 2 | 234078810 | junction | blood | 17.01 |
| 616 | ENST00000493871.1 | MPP1 | X | 154014479 | junction | blood | 6.09 |
| 617 | ENST00000494080.5 | CCDC88B | 11 | 64109214 | junction | blood | 7.88 |
| 618 | ENST00000494268.1 | MSL3 | X | 11778428 | junction | blood | 6.22 |
| 619 | ENST00000495406.1 | CDK5RAP2 | 9 | 123152019 | junction | blood | 10.53 |
| 620 | ENST00000496379.5 | FES | 15 | 91433192 | junction | blood | 7.77 |
| 621 | ENST00000496915.1 | PREX1 | 20 | 47258945 | junction | blood | 5.23 |
| 622 | ENST00000497441.5 | RASGRP2 | 11 | 64504225 | junction | blood | 12.78 |
| 623 | ENST00000497615.1 | NADK | 1 | 1685983 | junction | blood | 9.43 |
| 624 | ENST00000498461.1 | ALOX5 | 10 | 45938985 | junction | blood | 13.87 |
| 625 | ENST00000498666.5 | ITGB2 | 21 | 46340736 | junction | blood | 13.12 |
| 626 | ENST00000500323.2 | DOK3 | 5 | 176935238 | junction | blood | 10.88 |
| 627 | ENST00000502380.1 | DOK3 | 5 | 176937261 | junction | blood | 8.44 |
| 628 | ENST00000504910.1 | HK3 | 5 | 176318389 | junction | blood | 6.36 |
| 629 | ENST00000505207.5 | UBE2D3 | 4 | 103746879 | junction | blood | 7.43 |
| 630 | ENST00000509314.5 | FBXL5 | 4 | 15661350 | junction | blood | 10.24 |
| 631 | ENST00000509339.1 | MXD3 | 5 | 176734782 | junction | blood | 30.07 |
| 632 | ENST00000512426.5 | CCND3 | 6 | 41908108 | junction | blood | 7.01 |
| 633 | ENST00000512747.3 | IL2RG | X | 70331275 | junction | blood | 6.21 |
| 634 | ENST00000513001.5 | ACSL1 | 4 | 185678973 | junction | blood | 17.65 |
| 635 | ENST00000517804.5 | DMTN | 8 | 21914647 | junction | blood | 7.30 |
| 636 | ENST00000517813.1 | DENND3 | 8 | 142195377 | junction | blood | 13.47 |
| 637 | ENST00000517985.5 | DENND3 | 8 | 142188295 | junction | blood | 9.36 |
| 638 | ENST00000518135.1 | TNFRSF10C | 8 | 22972283 | junction | blood | 5.06 |
| 639 | ENST00000518249.5 | DENND3 | 8 | 142151423 | junction | blood | 9.51 |
| 640 | ENST00000519594.5 | LCP2 | 5 | 169724538 | junction | blood | 5.37 |
| 641 | ENST00000520322.1 | LCP2 | 5 | 169677734 | junction | blood | 5.68 |
| 642 | ENST00000520450.5 | DOCK2 | 5 | 169484670 | junction | blood | 11.23 |
| 643 | ENST00000520571.1 | DENND3 | 8 | 142199245 | junction | blood | 8.57 |
| 644 | ENST00000520887.1 | FAM49B | 8 | 130859050 | junction | blood | 7.15 |
| 645 | ENST00000521442.1 | ATP6V1B2 | 8 | 20075793 | junction | blood | 8.33 |
| 646 | ENST00000523015.1 | DENND3 | 8 | 142154358 | junction | blood | 7.05 |
| 647 | ENST00000523022.5 | CA1 | 8 | 86290275 | junction | blood | 40.92 |
| 648 | ENST00000523066.1 | DENND3 | 8 | 142183840 | junction | blood | 6.75 |
| 649 | ENST00000523266.5 | DMTN | 8 | 21917007 | junction | blood | 6.93 |
| 650 | ENST00000523369.1 | LCP2 | 5 | 169685041 | junction | blood | 9.88 |
| 651 | ENST00000523530.1 | DENND3 | 8 | 142202548 | junction | blood | 7.23 |
| 652 | ENST00000523615.1 | STK10 | 5 | 171488143 | junction | blood | 12.61 |
| 653 | ENST00000526387.5 | TBC1D10C | 11 | 67171411 | junction | blood | 7.10 |
| 654 | ENST00000526474.1 | TBC1D10C | 11 | 67172977 | junction | blood | 8.87 |
| 655 | ENST00000527031.5 | JAK3 | 19 | 17958755 | junction | blood | 6.17 |
| 656 | ENST00000527146.1 | IFITM2 | 11 | 308438 | junction | blood | 18.56 |
| 657 | ENST00000527525.5 | SIPA1 | 11 | 65407492 | junction | blood | 5.39 |
| 658 | ENST00000528086.5 | CD44 | 11 | 35160917 | junction | blood | 8.17 |
| 659 | ENST00000528780.5 | IFITM1 | 11 | 313691 | junction | blood | 47.88 |
| 660 | ENST00000530119.5 | TALDO1 | 11 | 747578 | junction | blood | 7.74 |
| 661 | ENST00000530226.1 | SIPA1 | 11 | 65413428 | junction | blood | 6.84 |
| 662 | ENST00000530587.5 | PHF21A | 11 | 45959708 | junction | blood | 6.25 |
| 663 | ENST00000531334.1 | ECE1 | 1 | 21548240 | junction | blood | 5.78 |
| 664 | ENST00000532028.1 | PHF21A | 11 | 45967391 | junction | blood | 5.50 |
| 665 | ENST00000532362.1 | CMTM2 | 16 | 66621001 | junction | blood | 114.27 |
| 666 | ENST00000532897.5 | MKNK1 | 1 | 47037735 | junction | blood | 6.23 |
| 667 | ENST00000533815.2 | BTNL8 | 5 | 180336697 | junction | blood | 5.72 |
| 668 | ENST00000533968.1 | SPI1 | 11 | 47399860 | junction | blood | 12.90 |
| 669 | ENST00000534261.3 | SIGLEC5 | 19 | 52148730 | junction | blood | 6.14 |
| 670 | ENST00000534754.5 | SORL1 | 11 | 121483541 | junction | blood | 9.84 |
| 671 | ENST00000535615.5 | CARS2 | 13 | 111317875 | junction | blood | 5.33 |
| 672 | ENST00000536885.1 | ARAP1 | 11 | 72399500 | junction | blood | 7.74 |
| 673 | ENST00000536995.5 | ATG16L2 | 11 | 72533340 | junction | blood | 11.55 |
| 674 | ENST00000538842.1 | ATG16L2 | 11 | 72527872 | junction | blood | 6.35 |
| 675 | ENST00000539134.1 | RELT | 11 | 73104962 | junction | blood | 6.58 |
| 676 | ENST00000540182.5 | NLRC5 | 16 | 57074630 | junction | blood | 6.20 |
| 677 | ENST00000540513.1 | ACRBP | 12 | 6747841 | junction | blood | 9.23 |
| 678 | ENST00000541206.6 | RNF166 | 16 | 88769198 | junction | blood | 11.36 |
| 679 | ENST00000541800.6 | PLBD1 | 12 | 14688593 | junction | blood | 6.78 |
| 680 | ENST00000542481.1 | ATG16L2 | 11 | 72535167 | junction | blood | 25.18 |
| 681 | ENST00000542587.5 | LPAR2 | 19 | 19739237 | junction | blood | 10.95 |
| 682 | ENST00000543576.5 | DENND1C | 19 | 6481690 | junction | blood | 12.06 |
| 683 | ENST00000544490.5 | ATG16L2 | 11 | 72525608 | junction | blood | 30.13 |
| 684 | ENST00000544997.5 | FERMT3 | 11 | 63974286 | junction | blood | 7.56 |
| 685 | ENST00000546704.1 | ARHGAP9 | 12 | 57868658 | junction | blood | 19.20 |
| 686 | ENST00000548139.5 | ARHGAP9 | 12 | 57871784 | junction | blood | 15.20 |
| 687 | ENST00000552953.5 | ARHGAP9 | 12 | 57870373 | junction | blood | 24.58 |
| 688 | ENST00000553070.5 | NFE2 | 12 | 54694585 | junction | blood | 23.61 |
| 689 | ENST00000553872.1 | PYGL | 14 | 51387674 | junction | blood | 8.64 |
| 690 | ENST00000554736.5 | GNG2 | 14 | 52328108 | junction | blood | 9.30 |
| 691 | ENST00000556418.1 | RIN3 | 14 | 93119420 | junction | blood | 12.65 |
| 692 | ENST00000558177.5 | ANPEP | 15 | 90345638 | junction | blood | 11.96 |
| 693 | ENST00000558332.3 | IL16 | 15 | 81592816 | junction | blood | 7.66 |
| 694 | ENST00000558588.5 | PLCB2 | 15 | 40599790 | junction | blood | 12.82 |
| 695 | ENST00000559341.5 | MAN2A2 | 15 | 91459486 | junction | blood | 8.61 |
| 696 | ENST00000560064.1 | PSTPIP1 | 15 | 77328276 | junction | blood | 5.96 |
| 697 | ENST00000561175.1 | ADAM8 | 10 | 135085920 | junction | blood | 6.86 |
| 698 | ENST00000562129.1 | CORO1A | 16 | 30199570 | junction | blood | 14.38 |
| 699 | ENST00000562759.1 | ST20 | 15 | 80199971 | junction | blood | 5.59 |
| 700 | ENST00000562918.5 | ITGAX | 16 | 31366612 | junction | blood | 10.15 |
| 701 | ENST00000563269.1 | PLCG2 | 16 | 81965259 | junction | blood | 6.38 |
| 702 | ENST00000563526.5 | CYBA | 16 | 88717364 | junction | blood | 7.13 |
| 703 | ENST00000564072.1 | SLCO3A1 | 15 | 92706776 | junction | blood | 8.74 |
| 704 | ENST00000564572.1 | CNN2 | 19 | 1039063 | junction | blood | 5.34 |
| 705 | ENST00000564632.1 | ITGAL | 16 | 30531288 | junction | blood | 12.98 |
| 706 | ENST00000564707.1 | AHSP | 16 | 31539335 | junction | blood | 6.85 |
| 707 | ENST00000564768.1 | CORO1A | 16 | 30198544 | junction | blood | 6.01 |
| 708 | ENST00000564894.1 | RNF166 | 16 | 88767670 | junction | blood | 7.90 |
| 709 | ENST00000564905.1 | XPO6 | 16 | 28145162 | junction | blood | 12.98 |
| 710 | ENST00000565076.1 | SIRPB1 | 20 | 1552366 | junction | blood | 5.74 |
| 711 | ENST00000565284.5 | XPO6 | 16 | 28163980 | junction | blood | 14.84 |
| 712 | ENST00000565497.5 | CORO1A | 16 | 30195046 | junction | blood | 6.42 |
| 713 | ENST00000566082.1 | USB1 | 16 | 58051343 | junction | blood | 6.82 |
| 714 | ENST00000567034.5 | CORO1A | 16 | 30198041 | junction | blood | 11.54 |
| 715 | ENST00000567038.1 | XPO6 | 16 | 28113168 | junction | blood | 8.97 |
| 716 | ENST00000567178.1 | ITGAM | 16 | 31339551 | junction | blood | 23.09 |
| 717 | ENST00000568760.5 | DEF8 | 16 | 90024068 | junction | blood | 7.24 |
| 718 | ENST00000570106.6 | SIGLEC5 | 19 | 52133552 | junction | blood | 40.53 |
| 719 | ENST00000570439.1 | ACAP1 | 17 | 7253547 | junction | blood | 5.27 |
| 720 | ENST00000570473.5 | TRIM25 | 17 | 54981616 | junction | blood | 5.32 |
| 721 | ENST00000570755.1 | MMP25 | 16 | 3097548 | junction | blood | 31.89 |
| 722 | ENST00000571206.1 | ARRB2 | 17 | 4619328 | junction | blood | 26.58 |
| 723 | ENST00000571220.1 | ACAP1 | 17 | 7246881 | junction | blood | 29.18 |
| 724 | ENST00000571303.1 | MLKL | 16 | 74725175 | junction | blood | 5.93 |
| 725 | ENST00000571791.5 | ARRB2 | 17 | 4614039 | junction | blood | 13.94 |
| 726 | ENST00000572418.1 | P2RX1 | 17 | 3819383 | junction | blood | 9.95 |
| 727 | ENST00000572666.5 | CORO7 | 16 | 4411362 | junction | blood | 7.67 |
| 728 | ENST00000572782.1 | ARRB2 | 17 | 4618338 | junction | blood | 16.63 |
| 729 | ENST00000574548.1 | RNF167 | 17 | 4847933 | junction | blood | 6.40 |
| 730 | ENST00000575441.1 | MMP25 | 16 | 3107210 | junction | blood | 88.59 |
| 731 | ENST00000576594.1 | ACAP1 | 17 | 7250561 | junction | blood | 17.88 |
| 732 | ENST00000576628.1 | ACAP1 | 17 | 7240106 | junction | blood | 20.20 |
| 733 | ENST00000576769.1 | SLC43A2 | 17 | 1486498 | junction | blood | 5.40 |
| 734 | ENST00000578574.1 | SLC16A3 | 17 | 80186982 | junction | blood | 7.93 |
| 735 | ENST00000581287.5 | SLC16A3 | 17 | 80194107 | junction | blood | 11.97 |
| 736 | ENST00000581974.1 | DHRS13 | 17 | 27226145 | junction | blood | 5.97 |
| 737 | ENST00000582174.5 | FLOT2 | 17 | 27224544 | junction | blood | 6.46 |
| 738 | ENST00000582812.5 | NDEL1 | 17 | 8316587 | junction | blood | 9.15 |
| 739 | ENST00000583093.5 | SECTM1 | 17 | 80291576 | junction | blood | 7.78 |
| 740 | ENST00000583810.5 | PIK3R5 | 17 | 8785105 | junction | blood | 8.43 |
| 741 | ENST00000584445.5 | NARF | 17 | 80439087 | junction | blood | 11.89 |
| 742 | ENST00000585852.5 | FMNL1 | 17 | 43308054 | junction | blood | 19.81 |
| 743 | ENST00000586080.1 | R3HDM4 | 19 | 900829 | junction | blood | 5.15 |
| 744 | ENST00000587259.5 | VMP1 | 17 | 57807378 | junction | blood | 10.58 |
| 745 | ENST00000587265.1 | CA4 | 17 | 58235488 | junction | blood | 5.45 |
| 746 | ENST00000587287.1 | RASGRP4 | 19 | 38901755 | junction | blood | 6.80 |
| 747 | ENST00000587444.1 | VASP | 19 | 46029261 | junction | blood | 6.41 |
| 748 | ENST00000587856.1 | FMNL1 | 17 | 43311567 | junction | blood | 22.20 |
| 749 | ENST00000588708.5 | RASGRP4 | 19 | 38907692 | junction | blood | 24.68 |
| 750 | ENST00000589261.5 | ICAM3 | 19 | 10450215 | junction | blood | 6.41 |
| 751 | ENST00000589911.1 | FMNL1 | 17 | 43322783 | junction | blood | 7.99 |
| 752 | ENST00000590227.5 | DNAH17 | 17 | 76445496 | junction | blood | 5.85 |
| 753 | ENST00000590856.1 | UNC13D | 17 | 73826659 | junction | blood | 6.33 |
| 754 | ENST00000590974.1 | LYL1 | 19 | 13213345 | junction | blood | 5.48 |
| 755 | ENST00000591616.1 | UNC13D | 17 | 73831001 | junction | blood | 7.12 |
| 756 | ENST00000592527.1 | FMNL1 | 17 | 43310664 | junction | blood | 7.31 |
| 757 | ENST00000594221.5 | STXBP2 | 19 | 7706739 | junction | blood | 6.21 |
| 758 | ENST00000594696.1 | PRAM1 | 19 | 8555984 | junction | blood | 17.63 |
| 759 | ENST00000594743.1 | CD37 | 19 | 49840478 | junction | blood | 8.07 |
| 760 | ENST00000595046.1 | MYO1F | 19 | 8641326 | junction | blood | 6.90 |
| 761 | ENST00000595325.5 | MYO1F | 19 | 8642191 | junction | blood | 13.66 |
| 762 | ENST00000595596.1 | GRAMD1A | 19 | 35504595 | junction | blood | 5.53 |
| 763 | ENST00000595725.5 | CD37 | 19 | 49838866 | junction | blood | 20.04 |
| 764 | ENST00000596937.1 | MYO1F | 19 | 8615044 | junction | blood | 6.21 |
| 765 | ENST00000597222.1 | MYO1F | 19 | 8604831 | junction | blood | 29.52 |
| 766 | ENST00000597611.7 | FKBP8 | 19 | 18654719 | junction | blood | 9.25 |
| 767 | ENST00000598005.1 | MYO1F | 19 | 8606797 | junction | blood | 17.89 |
| 768 | ENST00000598201.5 | SHKBP1 | 19 | 41086391 | junction | blood | 12.36 |
| 769 | ENST00000598529.5 | MYO1F | 19 | 8619361 | junction | blood | 16.08 |
| 770 | ENST00000598907.5 | SHKBP1 | 19 | 41084448 | junction | blood | 14.71 |
| 771 | ENST00000599123.1 | MYO1F | 19 | 8620543 | junction | blood | 7.54 |
| 772 | ENST00000599180.2 | FFAR2 | 19 | 35939281 | junction | blood | 24.79 |
| 773 | ENST00000599662.1 | CEACAM3 | 19 | 42312968 | junction | blood | 9.48 |
| 774 | ENST00000599694.1 | RASAL3 | 19 | 15567313 | junction | blood | 6.37 |
| 775 | ENST00000599716.5 | SHKBP1 | 19 | 41082891 | junction | blood | 10.66 |
| 776 | ENST00000600463.1 | IFI30 | 19 | 18286032 | junction | blood | 6.41 |
| 777 | ENST00000600885.1 | MYO1F | 19 | 8592222 | junction | blood | 5.95 |
| 778 | ENST00000601502.1 | MYO1F | 19 | 8610534 | junction | blood | 8.05 |
| 779 | ENST00000602101.6 | RASAL3 | 19 | 15575316 | junction | blood | 7.16 |
| 780 | ENST00000602136.1 | MYO1F | 19 | 8612920 | junction | blood | 7.01 |
| 781 | ENST00000602166.1 | EGLN2 | 19 | 41313179 | junction | blood | 5.19 |
| 782 | ENST00000602612.5 | KCNAB2 | 1 | 6086505 | junction | blood | 5.03 |
| 783 | ENST00000605039.5 | BIN2 | 12 | 51717806 | junction | blood | 11.02 |
| 784 | ENST00000607855.5 | CD177 | 19 | 43857918 | junction | blood | 5.94 |
| 785 | ENST00000609870.5 | CELF2 | 10 | 11207645 | junction | blood | 12.41 |
| 786 | ENST00000611028.2 | FOLR3 | 11 | 71846814 | junction | blood | 11.13 |
| 787 | ENST00000614135.4 | RASGRP4 | 19 | 38916709 | junction | blood | 8.18 |
| 788 | ENST00000614254.1 | AOAH | 7 | 36570024 | junction | blood | 14.33 |
| 789 | ENST00000616356.4 | FCN1 | 9 | 137809615 | junction | blood | 16.99 |
| 790 | ENST00000619100.4 | SLC38A5 | X | 48325349 | junction | blood | 10.86 |
| 791 | ENST00000621510.1 | AOAH | 7 | 36554078 | junction | blood | 19.27 |
| 792 | ENST00000622764.1 | SAP25 | 7 | 100171220 | junction | blood | 5.94 |

Columns
ID: feature index
Transcript: transcript ID
Gene: gene name
Chr: chromosome
Site: coordinate of nucleosome-depleted site (GRCh37)
Region: location of nucleosome-depleted site
Group: gene group based on its expression in blood and tumor
FPKM_{blood}: FPKM value in normal blood. Their FPKM values in tumors of 20 cancer types are all <1.

TABLE S11
Observed ctDNA fractions in the LOD
analysis for the CRC + BRCA model.

| | | Expected ctDNA | Observed ctDNA |
|---|---|---|---|
| Sample | Group | fractions | fraction |
| 1279_221015-0.005 | positive | 0.0050 | 0.0194 |
| 1279_221015-0.010 | positive | 0.0100 | 0.0245 |
| 1279_221015-0.015 | positive | 0.0150 | 0.0364 |
| 1279_221015-0.020 | positive | 0.0200 | 0.0361 |
| 1279_221015-0.025 | positive | 0.0250 | 0.0482 |
| 1279_221015-0.030 | positive | 0.0300 | 0.0515 |
| 1279_221015-0.035 | positive | 0.0350 | 0.0597 |
| 1279_221015-0.040 | positive | 0.0400 | 0.0721 |
| 1279_221015-0.045 | positive | 0.0450 | 0.0631 |
| 1279_221015-0.050 | positive | 0.0500 | 0.0652 |
| 1279_221015-0.075 | positive | 0.0750 | 0.0837 |
| 1279_221015-0.100 | positive | 0.1000 | 0.1084 |
| 1279_221015-0.125 | positive | 0.1250 | 0.1332 |
| 1279_221015-0.150 | positive | 0.1500 | 0.1566 |
| 1279_221015-0.175 | positive | 0.1750 | 0.1561 |
| 1279_221015-0.200 | positive | 0.2000 | 0.1770 |
| 1279_221015-0.225 | positive | 0.2250 | 0.1910 |
| 1279_221015-0.250 | positive | 0.2500 | 0.2421 |
| 1279_221015-0.275 | positive | 0.2750 | 0.2313 |
| 1279_221015-0.300 | positive | 0.3000 | 0.2821 |
| 1279_221015-0.325 | positive | 0.3250 | 0.3011 |
| 1279_221015-0.350 | positive | 0.3500 | 0.3237 |
| 1279_221015-0.375 | positive | 0.3750 | 0.3630 |
| 1279_221015-0.400 | positive | 0.4000 | 0.3893 |
| 1279_221015-0.425 | positive | 0.4250 | 0.4190 |
| 1279_221015-0.450 | positive | 0.4500 | 0.4658 |
| 1279_221015-0.475 | positive | 0.4750 | 0.4764 |
| 1279_221015-0.500 | positive | 0.5000 | 0.4699 |
| 1279_241116-0.005 | positive | 0.0050 | 0.0252 |
| 1279_241116-0.010 | positive | 0.0100 | 0.0143 |
| 1279_241116-0.015 | positive | 0.0150 | 0.0230 |
| 1279_241116-0.020 | positive | 0.0200 | 0.0242 |
| 1279_241116-0.025 | positive | 0.0250 | 0.0385 |
| 1279_241116-0.030 | positive | 0.0300 | 0.0310 |
| 1279_241116-0.035 | positive | 0.0350 | 0.0426 |
| 1279_241116-0.040 | positive | 0.0400 | 0.0407 |
| 1279_241116-0.045 | positive | 0.0450 | 0.0418 |
| 1279_241116-0.050 | positive | 0.0500 | 0.0457 |
| 1279_241116-0.075 | positive | 0.0750 | 0.0761 |
| 1279_241116-0.100 | positive | 0.1000 | 0.1275 |
| 1279_241116-0.125 | positive | 0.1250 | 0.1369 |
| 1279_241116-0.150 | positive | 0.1500 | 0.1199 |
| 1279_241116-0.175 | positive | 0.1750 | 0.1242 |
| 1279_241116-0.200 | positive | 0.2000 | 0.1394 |
| 1279_241116-0.225 | positive | 0.2250 | 0.1577 |
| 1279_241116-0.250 | positive | 0.2500 | 0.1860 |
| 1279_241116-0.275 | positive | 0.2750 | 0.2301 |
| 1279_241116-0.300 | positive | 0.3000 | 0.2666 |
| 1279_241116-0.325 | positive | 0.3250 | 0.3036 |
| 1279_241116-0.350 | positive | 0.3500 | 0.2946 |
| 1279_241116-0.375 | positive | 0.3750 | 0.3256 |
| 1279_241116-0.400 | positive | 0.4000 | 0.3788 |
| 1279_241116-0.425 | positive | 0.4250 | 0.4033 |
| 1279_241116-0.450 | positive | 0.4500 | 0.4591 |
| 1279_241116-0.475 | positive | 0.4750 | 0.4359 |
| 1279_241116-0.490 | positive | 0.4900 | 0.4611 |
| 512_051015-0.005 | positive | 0.0050 | 0.0273 |
| 512_051015-0.010 | positive | 0.0100 | 0.0221 |
| 512_051015-0.015 | positive | 0.0150 | 0.0301 |
| 512_051015-0.020 | positive | 0.0200 | 0.0252 |
| 512_051015-0.025 | positive | 0.0250 | 0.0283 |
| 512_051015-0.030 | positive | 0.0300 | 0.0233 |
| 512_051015-0.035 | positive | 0.0350 | 0.0485 |
| 512_051015-0.040 | positive | 0.0400 | 0.0712 |
| 512_051015-0.045 | positive | 0.0450 | 0.0793 |
| 512_051015-0.050 | positive | 0.0500 | 0.0947 |
| 512_051015-0.075 | positive | 0.0750 | 0.1269 |
| 512_051015-0.100 | positive | 0.1000 | 0.1528 |
| 512_051015-0.125 | positive | 0.1250 | 0.1788 |
| 512_051015-0.150 | positive | 0.1500 | 0.2018 |
| 512_051015-0.175 | positive | 0.1750 | 0.2097 |
| 512_051015-0.200 | positive | 0.2000 | 0.2359 |
| 512_051015-0.225 | positive | 0.2250 | 0.2709 |
| 512_051015-0.250 | positive | 0.2500 | 0.2759 |
| 512_051015-0.275 | positive | 0.2750 | 0.2731 |
| 512_051015-0.300 | positive | 0.3000 | 0.2930 |
| 512_051015-0.325 | positive | 0.3250 | 0.3230 |
| 512_051015-0.350 | positive | 0.3500 | 0.3364 |
| 512_051015-0.375 | positive | 0.3750 | 0.3723 |
| 512_051015-0.400 | positive | 0.4000 | 0.4192 |
| 512_051015-0.425 | positive | 0.4250 | 0.4303 |
| 512_051015-0.450 | positive | 0.4500 | 0.4825 |
| 512_051015-0.475 | positive | 0.4750 | 0.4897 |
| 512_051015-0.500 | positive | 0.5000 | 0.5015 |
| 512_051015-0.525 | positive | 0.5250 | 0.5312 |
| 512_051015-0.550 | positive | 0.5500 | 0.5683 |
| 512_051015-0.575 | positive | 0.5750 | 0.5989 |
| 512_051015-0.600 | positive | 0.6000 | 0.6028 |
| 512_051015-0.614 | positive | 0.6140 | 0.5969 |
| 512_130114-0.005 | positive | 0.0050 | 0.0308 |
| 512_130114-0.010 | positive | 0.0100 | 0.0283 |
| 512_130114-0.015 | positive | 0.0150 | 0.0118 |
| 512_130114-0.020 | positive | 0.0200 | 0.0000 |
| 512_130114-0.025 | positive | 0.0250 | 0.0167 |
| 512_130114-0.030 | positive | 0.0300 | 0.0283 |
| 512_130114-0.035 | positive | 0.0350 | 0.0565 |
| 512_130114-0.040 | positive | 0.0400 | 0.0616 |
| 512_130114-0.045 | positive | 0.0450 | 0.0454 |
| 512_130114-0.050 | positive | 0.0500 | 0.0398 |
| 512_130114-0.075 | positive | 0.0750 | 0.0774 |
| 512_130114-0.100 | positive | 0.1000 | 0.0893 |
| 512_130114-0.125 | positive | 0.1250 | 0.1541 |
| 512_130114-0.150 | positive | 0.1500 | 0.1891 |
| 512_130114-0.175 | positive | 0.1750 | 0.1988 |
| 512_130114-0.200 | positive | 0.2000 | 0.2222 |
| 512_130114-0.225 | positive | 0.2250 | 0.2824 |
| 512_130114-0.250 | positive | 0.2500 | 0.3523 |
| 512_130114-0.275 | positive | 0.2750 | 0.3871 |
| 512_130114-0.300 | positive | 0.3000 | 0.4119 |
| 512_130114-0.325 | positive | 0.3250 | 0.4908 |
| 512_130114-0.350 | positive | 0.3500 | 0.5099 |
| 512_130114-0.375 | positive | 0.3750 | 0.5502 |
| 512_130114-0.394 | positive | 0.3940 | 0.5966 |
| D19-0.005 | positive | 0.0050 | 0.0226 |
| D19-0.010 | positive | 0.0100 | 0.0232 |
| D19-0.015 | positive | 0.0150 | 0.0205 |
| D19-0.020 | positive | 0.0200 | 0.0316 |
| D19-0.025 | positive | 0.0250 | 0.0438 |
| D19-0.030 | positive | 0.0300 | 0.0458 |
| D19-0.035 | positive | 0.0350 | 0.0525 |
| D19-0.040 | positive | 0.0400 | 0.0458 |
| D19-0.045 | positive | 0.0450 | 0.0405 |
| D19-0.050 | positive | 0.0500 | 0.0400 |
| D19-0.075 | positive | 0.0750 | 0.0745 |
| D19-0.100 | positive | 0.1000 | 0.1132 |
| D19-0.125 | positive | 0.1250 | 0.1275 |
| D19-0.150 | positive | 0.1500 | 0.1471 |
| D19-0.175 | positive | 0.1750 | 0.1751 |
| D19-0.200 | positive | 0.2000 | 0.1870 |
| D19-0.225 | positive | 0.2250 | 0.1984 |
| D19-0.250 | positive | 0.2500 | 0.2162 |
| D19-0.275 | positive | 0.2750 | 0.2241 |
| D19-0.300 | positive | 0.3000 | 0.2362 |
| D19-0.325 | positive | 0.3250 | 0.2530 |
| D19-0.350 | positive | 0.3500 | 0.3078 |
| D19-0.375 | positive | 0.3750 | 0.3103 |
| D19-0.400 | positive | 0.4000 | 0.3277 |
| D19-0.425 | positive | 0.4250 | 0.3472 |
| D19-0.450 | positive | 0.4500 | 0.3787 |
| D19-0.475 | positive | 0.4750 | 0.3919 |
| D19-0.500 | positive | 0.5000 | 0.4156 |
| D19-0.525 | positive | 0.5250 | 0.4327 |
| D19-0.550 | positive | 0.5500 | 0.4640 |
| D19-0.575 | positive | 0.5750 | 0.4798 |
| D19-0.600 | positive | 0.6000 | 0.5154 |
| D19-0.625 | positive | 0.6250 | 0.5406 |
| D19-0.650 | positive | 0.6500 | 0.5359 |
| D19-0.675 | positive | 0.6750 | 0.5388 |
| D19-0.700 | positive | 0.7000 | 0.5457 |
| D19-0.725 | positive | 0.7250 | 0.5634 |
| D19-0.7363 | positive | 0.7363 | 0.5554 |
| E2c-0.005 | positive | 0.0050 | 0.0033 |
| E2c-0.010 | positive | 0.0100 | 0.0041 |
| E2c-0.015 | positive | 0.0150 | 0.0371 |
| E2c-0.020 | positive | 0.0200 | 0.0500 |
| E2c-0.025 | positive | 0.0250 | 0.0740 |
| E2c-0.030 | positive | 0.0300 | 0.0815 |
| E2c-0.035 | positive | 0.0350 | 0.0885 |
| E2c-0.040 | positive | 0.0400 | 0.0859 |
| E2c-0.045 | positive | 0.0450 | 0.0961 |
| E2c-0.050 | positive | 0.0500 | 0.1203 |
| E2c-0.075 | positive | 0.0750 | 0.1333 |
| E2c-0.100 | positive | 0.1000 | 0.1525 |
| E2c-0.125 | positive | 0.1250 | 0.1676 |
| E2c-0.150 | positive | 0.1500 | 0.1783 |
| E2c-0.175 | positive | 0.1750 | 0.2136 |
| E2c-0.200 | positive | 0.2000 | 0.2138 |
| E2c-0.225 | positive | 0.2250 | 0.2539 |
| E2c-0.250 | positive | 0.2500 | 0.3305 |
| E2c-0.275 | positive | 0.2750 | 0.3979 |
| E2c-0.300 | positive | 0.3000 | 0.4422 |
| E2c-0.325 | positive | 0.3250 | 0.4868 |
| E2c-0.350 | positive | 0.3500 | 0.5341 |
| E2c-0.375 | positive | 0.3750 | 0.5800 |
| E2c-0.400 | positive | 0.4000 | 0.5747 |
| E2c-0.4122 | positive | 0.4122 | 0.5703 |
| E6c-0.005 | positive | 0.0050 | 0.0165 |
| E6c-0.010 | positive | 0.0100 | 0.0221 |
| E6c-0.015 | positive | 0.0150 | 0.0206 |
| E6c-0.020 | positive | 0.0200 | 0.0395 |
| E6c-0.025 | positive | 0.0250 | 0.0451 |
| E6c-0.030 | positive | 0.0300 | 0.0589 |
| E6c-0.035 | positive | 0.0350 | 0.0643 |
| E6c-0.040 | positive | 0.0400 | 0.0687 |
| E6c-0.045 | positive | 0.0450 | 0.0750 |
| E6c-0.050 | positive | 0.0500 | 0.0744 |
| E6c-0.075 | positive | 0.0750 | 0.1185 |
| E6c-0.100 | positive | 0.1000 | 0.1275 |
| E6c-0.125 | positive | 0.1250 | 0.1787 |
| E6c-0.150 | positive | 0.1500 | 0.2008 |
| E6c-0.175 | positive | 0.1750 | 0.2179 |
| E6c-0.200 | positive | 0.2000 | 0.2374 |
| E6c-0.225 | positive | 0.2250 | 0.2570 |
| E6c-0.250 | positive | 0.2500 | 0.3111 |
| E6c-0.275 | positive | 0.2750 | 0.3204 |
| E6c-0.300 | positive | 0.3000 | 0.3706 |
| E6c-0.325 | positive | 0.3250 | 0.4481 |
| E6c-0.350 | positive | 0.3500 | 0.4578 |
| E6c-0.375 | positive | 0.3750 | 0.4920 |
| E6c-0.400 | positive | 0.4000 | 0.5168 |
| E6c-0.425 | positive | 0.4250 | 0.5272 |
| E6c-0.450 | positive | 0.4500 | 0.5648 |
| E6c-0.475 | positive | 0.4750 | 0.5723 |
| E6c-0.500 | positive | 0.5000 | 0.6091 |
| E6c-0.525 | positive | 0.5250 | 0.6031 |
| E6c-0.5386 | positive | 0.5386 | 0.5896 |
| sub-healthy-1 | negative | 0.0000 | 0.0187 |
| sub-healthy-2 | negative | 0.0000 | 0.0122 |
| sub-healthy-3 | negative | 0.0000 | 0.0193 |
| sub-healthy-4 | negative | 0.0000 | 0.0179 |
| sub-healthy-5 | negative | 0.0000 | 0.0040 |
| sub-healthy-6 | negative | 0.0000 | 0.0188 |
| sub-healthy-7 | negative | 0.0000 | 0.0155 |
| sub-healthy-8 | negative | 0.0000 | 0.0157 |
| sub-healthy-9 | negative | 0.0000 | 0.0151 |
| sub-healthy-10 | negative | 0.0000 | 0.0148 |
| sub-healthy-11 | negative | 0.0000 | 0.0099 |
| sub-healthy-12 | negative | 0.0000 | 0.0241 |
| sub-healthy-13 | negative | 0.0000 | 0.0136 |
| sub-healthy-14 | negative | 0.0000 | 0.0129 |
| sub-healthy-15 | negative | 0.0000 | 0.0020 |
| sub-healthy-16 | negative | 0.0000 | 0.0050 |
| sub-healthy-17 | negative | 0.0000 | 0.0128 |
| sub-healthy-18 | negative | 0.0000 | 0.0087 |
| sub-healthy-19 | negative | 0.0000 | 0.0084 |
| sub-healthy-20 | negative | 0.0000 | 0.0012 |
| sub-healthy-21 | negative | 0.0000 | 0.0067 |
| sub-healthy-22 | negative | 0.0000 | 0.0218 |
| sub-healthy-23 | negative | 0.0000 | 0.0208 |
| sub-healthy-24 | negative | 0.0000 | 0.0138 |
| sub-healthy-25 | negative | 0.0000 | 0.0227 |
| sub-healthy-26 | negative | 0.0000 | 0.0079 |
| sub-healthy-27 | negative | 0.0000 | 0.0178 |
| sub-healthy-28 | negative | 0.0000 | 0.0262 |
| sub-healthy-29 | negative | 0.0000 | 0.0157 |
| sub-healthy-30 | negative | 0.0000 | 0.0195 |
| sub-healthy-31 | negative | 0.0000 | 0.0075 |
| sub-healthy-32 | negative | 0.0000 | 0.0011 |
| sub-healthy-33 | negative | 0.0000 | 0.0114 |
| sub-healthy-34 | negative | 0.0000 | 0.0045 |
| sub-healthy-35 | negative | 0.0000 | 0.0143 |
| sub-healthy-36 | negative | 0.0000 | 0.0131 |
| sub-healthy-37 | negative | 0.0000 | 0.0168 |
| sub-healthy-38 | negative | 0.0000 | 0.0065 |
| sub-healthy-39 | negative | 0.0000 | 0.0026 |
| sub-healthy-40 | negative | 0.0000 | 0.0028 |

TABLE S12
A panel of 77 genes for screening breast cancer samples.

| | | | | | | |
|---|---|---|---|---|---|---|
| CDH1 | ARID1A | MAGI3 | FLT3 | SMARCB1 | RNF43 | FGFR2 |
| CDKN2A | ATR | MDM2 | GNA11 | SMO | APC | MET |
| HRAS | AURKA | MDM4 | GNAQ | VHL | EGFR | FGFR3 |
| JAK2 | BRCA1 | KMT2C | GNAS | BRAF | ZNRF3 | ALK |
| JAK3 | BRCA2 | NOTCH4 | HNF1A | KRAS | ATM | MAP2K1 |
| NF1 | CDKN1B | TSC1 | IDH1 | NRAS | PTEN | MTOR |
| NOTCH1 | DOT1L | TSC2 | IDH2 | PIK3CA | CTNNB1 | MAP2K4 |
| RB1 | ERBB3 | ABL1 | KDR | PIK3R1 | MLH1 | MAP3K1 |
| RET | ESR1 | CSF1R | MPL | SMAD4 | ERBB2 | AKT1 |
| SRC | GATA3 | DDR2 | NPM1 | TP53 | ERBB4 | PDGFRA |
| STK11 | JAK1 | EZH2 | PTPN11 | FBXW7 | FGFR1 | KIT |

TABLE S13
Transcript expression data.

| | | sample | |
|---|---|---|---|
| source | tissue | size | download URLs |
| GTEx | whole blood | 337 | https://toil.xenahubs.net/download/gtex_RSEM_isoform_fpkm.gz |
| TCGA | tumor of colorectal | 372 | https://toil.xenahubs.net/download/tcga_RSEM_isoform_fpkm.gz |
| | cancer (CRC) | | |

TABLE S14
Information on all predictive features for colorectal cancer

| Feature ID | MAE |
|---|---|
| 59 86 475 703 760 790 | 0.017 |
| 70 256 268 322 379 903 | 0.019 |
| 72 86 703 760 790 900 | 0.019 |
| 70 78 268 322 364 475 | 0.020 |
| 86 553 703 760 790 900 | 0.020 |
| 59 86 703 760 790 900 | 0.020 |
| 72 86 760 790 900 905 | 0.020 |
| 256 268 322 379 475 553 | 0.020 |
| 70 268 322 364 571 903 | 0.021 |
| 70 161 268 322 571 903 | 0.021 |
| 161 268 322 359 553 903 | 0.021 |
| 86 703 760 790 900 905 | 0.021 |
| 78 268 284 322 571 903 | 0.021 |
| 256 268 284 322 359 379 | 0.021 |
| 115 161 268 744 903 905 | 0.021 |
| 70 78 268 284 322 903 | 0.021 |
| 161 268 284 359 553 903 | 0.021 |
| 70 115 268 322 571 903 | 0.021 |
| 284 355 379 475 571 788 | 0.021 |
| 70 268 284 322 571 903 | 0.021 |
| 59 78 126 154 181 639 | 0.021 |
| 86 703 760 788 790 900 | 0.021 |
| 70 161 181 322 480 903 | 0.021 |
| 59 86 703 760 790 905 | 0.021 |
| 124 181 256 485 744 905 | 0.021 |
| 70 161 268 284 322 903 | 0.022 |
| 70 115 161 268 359 903 | 0.022 |
| 115 161 268 553 884 905 | 0.022 |
| 124 161 268 553 673 905 | 0.022 |
| 70 268 284 322 364 903 | 0.022 |
| 59 124 126 153 161 250 | 0.022 |
| 70 124 161 268 322 903 | 0.022 |
| 115 161 181 553 673 905 | 0.022 |
| 161 268 322 359 553 571 | 0.022 |
| 124 163 268 485 553 673 | 0.022 |
| 70 268 571 839 903 905 | 0.022 |
| 78 268 284 322 884 903 | 0.022 |
| 268 322 359 379 475 553 | 0.022 |
| 124 181 485 571 673 744 | 0.022 |
| 268 284 322 553 884 903 | 0.022 |
| 78 115 124 268 322 903 | 0.022 |
| 124 284 475 480 673 788 | 0.022 |
| 126 568 604 714 777 830 | 0.022 |
| 70 161 268 284 903 905 | 0.022 |
| 59 86 553 703 760 790 | 0.022 |
| 268 322 379 475 553 905 | 0.022 |
| 67 124 161 181 284 673 | 0.022 |
| 268 359 379 475 553 905 | 0.023 |
| 70 268 284 322 884 903 | 0.023 |
| 115 284 359 485 571 777 | 0.023 |
| 268 284 359 379 475 553 | 0.023 |
| 268 322 777 788 884 903 | 0.023 |
| 70 181 322 475 480 884 | 0.023 |
| 78 161 258 234 322 903 | 0.023 |
| 115 161 268 284 903 905 | 0.023 |
| 115 124 181 284 447 485 | 0.023 |
| 115 181 284 571 824 903 | 0.023 |
| 70 124 161 268 673 903 | 0.023 |
| 67 124 181 485 673 744 | 0.023 |
| 67 124 181 673 830 839 | 0.023 |
| 70 268 284 322 673 903 | 0.023 |
| 70 124 161 268 903 905 | 0.023 |
| 70 161 268 839 903 905 | 0.023 |
| 70 115 124 268 903 905 | 0.023 |
| 59 86 703 760 788 790 | 0.023 |
| 39 447 466 595 604 639 | 0.023 |
| 124 161 181 256 673 744 | 0.023 |
| 70 470 480 485 639 858 | 0.023 |
| 70 115 181 475 571 884 | 0.023 |
| 268 359 379 475 553 788 | 0.023 |
| 70 115 268 379 571 900 | 0.023 |
| 70 124 161 181 903 905 | 0.023 |
| 72 154 284 777 788 884 | 0.024 |
| 124 161 268 553 903 305 | 0.024 |
| 336 540 632 714 793 874 | 0.024 |
| 70 181 336 830 839 884 | 0.024 |
| 70 115 124 268 571 903 | 0.024 |
| 2 256 336 529 632 682 | 0.024 |
| 161 256 268 336 475 905 | 0.024 |
| 379 553 571 788 858 900 | 0.024 |
| 268 284 322 673 788 903 | 0.024 |
| 115 124 161 181 284 447 | 0.024 |
| 70 268 284 788 903 905 | 0.024 |
| 115 284 480 485 529 777 | 0.024 |
| 359 475 553 571 788 900 | 0.024 |
| 70 161 181 571 884 903 | 0.024 |
| 161 181 744 884 903 905 | 0.024 |
| 70 161 181 284 884 903 | 0.024 |
| 115 124 268 322 379 673 | 0.024 |
| 268 284 322 788 884 903 | 0.024 |
| 82 217 477 487 629 717 | 0.024 |
| 268 359 379 475 571 788 | 0.024 |
| 161 268 284 322 673 903 | 0.024 |
| 126 466 470 639 793 858 | 0.024 |
| 2 39 256 395 619 839 | 0.024 |
| 124 126 161 181 284 673 | 0.024 |
| 2 115 324 181 359 830 | 0.025 |
| 359 379 475 553 571 900 | 0.025 |
| 124 161 181 673 744 830 | 0.025 |
| 70 151 268 284 673 903 | 0.025 |
| 124 181 256 359 485 830 | 0.025 |
| 124 181 364 673 788 903 | 0.025 |
| 115 161 181 359 485 673 | 0.025 |
| 480 485 673 721 744 858 | 0.025 |
| 70 475 571 788 900 905 | 0.025 |
| 284 379 475 788 900 905 | 0.025 |
| 78 153 322 568 830 884 | 0.025 |
| 70 379 475 571 788 900 | 0.025 |
| 124 161 181 284 364 673 | 0.025 |
| 115 124 161 181 673 744 | 0.025 |
| 2 337 384 395 839 899 | 0.025 |
| 161 256 268 359 379 573 | 0.025 |
| 284 322 788 884 903 905 | 0.025 |
| 115 161 268 284 673 903 | 0.025 |
| 364 410 525 542 793 839 | 0.025 |
| 70 115 124 161 181 905 | 0.025 |
| 124 268 284 322 788 903 | 0.025 |
| 115 154 359 480 485 830 | 0.025 |
| 78 154 364 542 619 796 | 0.025 |
| 84 336 359 466 632 858 | 0.025 |
| 359 379 475 553 788 884 | 0.025 |
| 379 553 571 788 900 905 | 0.025 |
| 72 126 204 542 714 796 | 0.025 |
| 78 124 181 359 485 830 | 0.025 |
| 181 256 359 485 777 830 | 0.025 |
| 268 359 379 475 788 884 | 0.025 |
| 115 124 161 268 903 905 | 0.025 |
| 78 115 154 364 604 830 | 0.025 |
| 359 379 475 553 571 788 | 0.025 |
| 181 256 485 555 673 744 | 0.025 |
| 124 181 256 284 673 830 | 0.025 |
| 115 126 161 284 903 905 | 0.025 |
| 78 154 364 485 568 830 | 0.025 |
| 39 256 525 568 619 839 | 0.025 |
| 115 154 181 256 485 905 | 0.026 |
| 181 202 284 322 673 884 | 0.026 |
| 78 204 364 525 542 632 | 0.026 |
| 181 284 322 673 777 884 | 0.026 |
| 115 161 181 673 744 884 | 0.026 |
| 364 563 619 830 858 884 | 0.026 |
| 284 322 571 788 884 903 | 0.026 |
| 181 284 322 480 903 905 | 0.026 |
| 115 124 161 181 284 673 | 0.026 |
| 32 122 202 286 487 594 | 0.026 |
| 115 161 268 322 884 903 | 0.026 |
| 72 153 247 250 591 594 | 0.026 |
| 70 115 161 268 234 903 | 0.026 |
| 70 181 478 721 744 884 | 0.026 |
| 115 124 181 673 744 830 | 0.026 |
| 70 124 181 788 903 905 | 0.026 |
| 284 359 379 475 553 571 | 0.026 |
| 78 154 542 568 721 830 | 0.026 |
| 78 126 256 522 542 639 | 0.026 |
| 161 268 284 322 359 903 | 0.026 |
| 124 161 268 322 379 673 | 0.026 |
| 67 332 455 580 704 904 | 0.026 |
| 268 322 359 571 788 903 | 0.026 |
| 2 256 464 540 568 874 | 0.026 |
| 154 480 485 568 673 744 | 0.026 |
| 124 161 268 673 788 903 | 0.026 |
| 70 395 410 540 702 874 | 0.026 |
| 161 268 322 364 673 903 | 0.026 |
| 124 181 256 359 744 830 | 0.026 |
| 181 284 322 485 721 903 | 0.026 |
| 59 336 470 632 721 858 | 0.026 |
| 39 256 337 407 702 839 | 0.026 |
| 67 124 181 284 364 903 | 0.026 |
| 359 619 721 744 830 884 | 0.026 |
| 181 284 322 359 485 884 | 0.026 |
| 268 284 322 359 379 903 | 0.026 |
| 124 181 284 359 364 485 | 0.026 |
| 268 379 475 571 788 900 | 0.026 |
| 70 124 126 256 470 830 | 0.026 |
| 2 115 154 604 619 830 | 0.026 |
| 115 154 359 480 721 777 | 0.026 |
| 153 256 336 359 410 639 | 0.026 |
| 84 359 604 619 830 858 | 0.026 |
| 39 204 632 793 858 901 | 0.026 |
| 67 78 124 126 256 364 | 0.026 |
| 322 359 379 475 788 884 | 0.027 |
| 59 161 181 673 744 884 | 0.027 |
| 181 364 673 721 744 903 | 0.027 |
| 181 284 322 485 673 777 | 0.027 |
| 78 204 632 682 721 858 | 0.027 |
| 161 181 322 673 744 884 | 0.027 |
| 115 124 161 181 673 905 | 0.027 |
| 70 364 480 542 619 721 | 0.027 |
| 70 379 475 788 900 905 | 0.027 |
| 268 322 359 379 673 903 | 0.027 |
| 96 122 202 447 675 695 | 0.027 |
| 59 78 115 525 619 744 | 0.027 |
| 322 359 379 553 571 788 | 0.027 |
| 59 124 161 181 322 903 | 0.027 |
| 78 154 250 478 777 796 | 0.027 |
| 322 359 379 475 571 788 | 0.027 |
| 284 359 379 475 553 788 | 0.027 |
| 154 181 284 322 673 777 | 0.027 |
| 124 268 359 379 475 571 | 0.027 |
| 161 268 284 322 884 903 | 0.027 |
| 70 480 542 639 682 721 | 0.027 |
| 161 181 202 284 884 903 | 0.027 |
| 78 124 161 268 284 903 | 0.027 |
| 78 124 181 673 744 830 | 0.027 |
| 115 322 604 744 777 830 | 0.027 |
| 70 124 181 256 284 903 | 0.027 |
| 124 359 379 475 788 905 | 0.027 |
| 139 250 336 364 542 796 | 0.027 |
| 322 359 480 673 721 744 | 0.027 |
| 154 364 480 485 542 619 | 0.027 |
| 78 126 364 632 777 796 | 0.027 |
| 59 639 682 793 796 858 | 0.027 |
| 204 478 542 639 721 793 | 0.027 |
| 67 468 487 721 780 899 | 0.027 |
| 451 522 525 542 619 793 | 0.027 |
| 126 161 181 673 903 905 | 0.027 |
| 39 237 384 702 727 796 | 0.027 |
| 124 161 368 571 673 903 | 0.027 |
| 124 161 181 359 485 744 | 0.027 |
| 126 154 466 639 682 858 | 0.027 |
| 115 410 525 744 777 830 | 0.027 |
| 59 161 181 202 480 744 | 0.027 |
| 379 475 553 571 788 900 | 0.027 |
| 35 39 153 464 540 796 | 0.027 |
| 161 181 485 673 744 884 | 0.027 |
| 59 73 126 153 154 884 | 0.027 |
| 126 336 348 466 721 852 | 0.027 |
| 115 124 480 673 744 905 | 0.027 |
| 115 181 485 673 744 839 | 0.027 |
| 78 126 154 485 568 777 | 0.027 |
| 78 182 284 322 485 777 | 0.027 |
| 78 154 181 284 485 777 | 0.027 |
| 322 359 475 553 571 788 | 0.027 |
| 59 161 268 322 364 903 | 0.027 |
| 2 39 337 702 766 899 | 0.027 |
| 204 364 542 619 714 796 | 0.027 |
| 70 379 475 571 900 905 | 0.027 |
| 67 153 429 540 568 874 | 0.027 |
| 359 379 475 788 884 905 | 0.027 |
| 59 124 161 181 673 903 | 0.027 |
| 78 154 364 568 777 830 | 0.027 |
| 70 115 124 268 485 903 | 0.027 |
| 359 379 475 571 788 905 | 0.027 |
| 72 109 202 348 410 473 | 0.027 |
| 256 447 468 469 568 717 | 0.028 |
| 18 217 391 455 480 717 | 0.028 |
| 268 284 379 475 788 900 | 0.028 |
| 42 407 522 568 721 896 | 0.028 |
| 70 72 542 639 744 796 | 0.028 |
| 70 126 181 478 571 839 | 0.028 |
| 70 126 470 480 639 858 | 0.028 |
| 59 124 161 359 485 830 | 0.028 |
| 2 18 410 525 632 839 | 0.028 |
| 59 475 553 673 703 788 | 0.028 |
| 204 256 525 568 682 793 | 0.028 |
| 181 284 322 485 777 884 | 0.028 |
| 70 153 322 568 830 884 | 0.028 |
| 115 124 447 702 830 858 | 0.028 |
| 78 126 154 639 793 858 | 0.028 |
| 67 451 455 470 568 766 | 0.028 |
| 395 540 632 714 793 874 | 0.028 |
| 39 204 480 542 632 793 | 0.028 |
| 59 379 475 571 673 788 | 0.028 |
| 379 475 553 788 884 905 | 0.028 |
| 78 115 256 470 788 830 | 0.028 |
| 161 268 673 788 903 905 | 0.028 |
| 124 181 322 364 485 673 | 0.028 |
| 359 379 475 553 571 884 | 0.028 |
| 126 529 632 639 793 858 | 0.028 |
| 18 466 639 744 858 884 | 0.028 |
| 268 379 475 788 900 905 | 0.028 |
| 410 466 470 480 525 744 | 0.028 |
| 78 115 154 485 744 830 | 0.028 |
| 115 154 181 359 485 903 | 0.028 |
| 447 468 529 704 841 867 | 0.028 |
| 39 337 714 766 793 842 | 0.028 |
| 70 256 336 395 632 839 | 0.028 |
| 161 268 284 485 673 903 | 0.028 |
| 78 154 181 485 777 839 | 0.028 |
| 139 384 455 525 568 830 | 0.028 |
| 78 154 359 455 480 830 | 0.028 |
| 154 395 466 555 639 796 | 0.028 |
| 67 197 270 429 632 761 | 0.028 |
| 78 154 322 364 568 777 | 0.028 |
| 32 179 473 753 780 904 | 0.028 |
| 115 124 161 268 364 839 | 0.028 |
| 72 154 284 485 673 777 | 0.028 |
| 154 359 480 485 673 830 | 0.028 |
| 204 466 470 480 639 858 | 0.028 |
| 59 126 410 619 639 744 | 0.028 |
| 161 268 284 322 379 903 | 0.028 |
| 256 407 473 555 824 874 | 0.028 |
| 2 522 525 529 721 793 | 0.028 |
| 359 379 475 553 571 673 | 0.028 |
| 78 256 632 682 777 858 | 0.028 |
| 67 115 124 256 284 485 | 0.028 |
| 59 480 485 744 830 884 | 0.028 |
| 78 115 153 322 721 777 | 0.028 |
| 153 525 542 714 721 796 | 0.028 |
| 379 475 553 571 788 884 | 0.028 |
| 78 115 154 181 284 777 | 0.028 |
| 115 284 485 571 673 884 | 0.028 |
| 115 284 485 529 777 884 | 0.028 |
| 126 466 470 714 796 858 | 0.028 |
| 84 204 466 470 604 858 | 0.028 |
| 115 154 284 364 571 884 | 0.028 |
| 70 115 181 478 744 777 | 0.028 |
| 181 336 480 702 721 744 | 0.028 |
| 115 154 673 744 830 858 | 0.028 |
| 322 359 379 475 553 788 | 0.028 |
| 78 115 154 256 777 830 | 0.028 |
| 78 250 256 364 410 796 | 0.028 |
| 204 364 470 568 721 830 | 0.029 |
| 70 364 470 480 639 793 | 0.029 |
| 78 204 542 632 766 858 | 0.029 |
| 78 126 153 154 529 830 | 0.029 |
| 115 124 126 161 485 555 | 0.029 |
| 447 451 522 721 793 842 | 0.029 |
| 59 78 126 154 744 777 | 0.029 |
| 59 126 714 839 874 884 | 0.029 |
| 18 256 522 555 632 682 | 0.029 |
| 115 480 485 619 744 830 | 0.029 |
| 359 379 475 553 673 788 | 0.029 |
| 466 528 540 717 824 904 | 0.029 |
| 264 270 412 447 842 856 | 0.029 |
| 395 451 470 568 604 656 | 0.029 |
| 39 337 384 466 639 824 | 0.029 |
| 540 604 632 688 780 904 | 0.029 |
| 364 485 542 796 839 905 | 0.029 |
| 379 475 571 788 900 905 | 0.029 |
| 79 96 217 336 717 753 | 0.029 |
| 126 154 256 359 830 884 | 0.029 |
| 78 126 154 571 639 858 | 0.029 |
| 379 475 673 788 884 905 | 0.029 |
| 332 580 704 796 841 904 | 0.029 |
| 96 204 256 384 466 470 | 0.029 |
| 115 124 161 181 485 673 | 0.029 |
| 78 153 410 542 632 766 | 0.029 |
| 124 268 379 571 788 900 | 0.029 |
| 78 154 485 744 777 858 | 0.029 |
| 18 139 325 407 466 632 | 0.029 |
| 115 154 364 480 619 858 | 0.029 |
| 337 429 555 714 822 824 | 0.029 |
| 2 70 395 540 632 682 | 0.029 |
| 161 181 284 485 673 721 | 0.029 |
| 79 336 455 466 632 702 | 0.029 |
| 72 96 468 477 590 855 | 0.029 |
| 359 379 475 553 788 905 | 0.029 |
| 161 268 788 884 903 905 | 0.029 |
| 2 395 540 632 714 874 | 0.029 |
| 249 337 522 704 749 780 | 0.029 |
| 2 33 337 478 525 540 | 0.029 |
| 79 204 429 522 525 529 | 0.029 |
| 35 270 293 555 632 896 | 0.029 |
| 153 154 480 568 682 884 | 0.029 |
| 284 485 571 673 777 884 | 0.029 |
| 115 124 161 268 364 903 | 0.029 |
| 126 395 451 542 714 793 | 0.029 |
| 18 153 447 528 632 904 | 0.029 |
| 359 379 475 788 900 905 | 0.029 |
| 88 293 412 428 632 685 | 0.029 |
| 412 404 466 632 704 842 | 0.029 |
| 35 79 249 704 780 896 | 0.029 |
| 78 115 126 154 256 777 | 0.029 |
| 4 85 202 217 249 465 | 0.029 |
| 153 568 604 714 727 766 | 0.029 |
| 379 475 553 571 673 788 | 0.029 |
| 2 391 466 632 735 858 | 0.029 |
| 78 154 364 604 777 858 | 0.029 |
| 35 67 270 384 412 727 | 0.029 |
| 2 18 96 153 555 842 | 0.029 |
| 67 522 529 540 688 842 | 0.029 |
| 202 204 466 470 639 682 | 0.029 |
| 18 204 384 466 639 899 | 0.029 |
| 249 395 487 594 841 899 | 0.029 |
| 70 153 336 455 568 721 | 0.029 |
| 67 336 429 451 470 480 | 0.029 |
| 124 161 268 364 571 903 | 0.029 |
| 59 115 126 485 744 830 | 0.029 |
| 322 359 379 475 553 571 | 0.029 |
| 78 115 124 256 322 485 | 0.029 |
| 67 217 230 249 465 487 | 0.029 |
| 466 528 540 555 717 824 | 0.030 |
| 39 204 525 542 619 793 | 0.030 |
| 2 256 451 568 824 874 | 0.030 |
| 202 256 469 473 528 717 | 0.030 |
| 115 181 256 284 364 903 | 0.030 |
| 176 266 407 447 522 727 | 0.030 |
| 39 256 395 447 555 748 | 0.030 |
| 115 124 161 268 744 830 | 0.030 |
| 67 395 412 451 464 727 | 0.030 |
| 39 337 466 766 842 899 | 0.030 |
| 154 181 322 673 788 884 | 0.030 |
| 2 126 359 777 796 858 | 0.030 |
| 70 284 322 384 571 903 | 0.030 |
| 78 115 153 154 721 777 | 0.030 |
| 67 75 217 465 511 899 | 0.030 |
| 42 391 407 451 466 717 | 0.030 |
| 18 153 204 542 568 796 | 0.030 |
| 124 181 284 485 777 884 | 0.030 |
| 256 470 632 766 777 796 | 0.030 |
| 126 395 447 632 714 793 | 0.030 |
| 407 466 522 528 604 639 | 0.030 |
| 78 124 181 364 485 673 | 0.030 |
| 78 124 364 542 639 714 | 0.030 |
| 379 475 553 571 788 905 | 0.030 |
| 39 153 395 540 721 727 | 0.030 |
| 70 395 470 485 766 899 | 0.030 |
| 39 204 395 528 604 839 | 0.030 |
| 79 337 395 528 555 727 | 0.030 |
| 379 475 571 673 788 884 | 0.030 |
| 379 475 571 673 788 905 | 0.030 |
| 153 154 480 542 721 793 | 0.030 |
| 35 391 540 586 717 793 | 0.030 |
| 475 553 571 788 884 905 | 0.030 |
| 275 359 485 619 796 830 | 0.030 |
| 18 122 202 270 688 761 | 0.030 |
| 67 72 410 429 704 901 | 0.030 |
| 82 154 364 793 796 858 | 0.030 |
| 124 268 379 553 788 805 | 0.030 |
| 466 528 568 721 727 766 | 0.030 |
| 35 57 632 634 655 904 | 0.030 |
| 18 154 470 540 639 796 | 0.030 |
| 57 82 202 410 594 632 | 0.030 |
| 179 249 270 293 337 515 | 0.030 |
| 204 604 619 714 777 796 | 0.030 |
| 70 126 364 542 619 796 | 0.030 |
| 217 467 477 478 717 841 | 0.030 |
| 18 67 487 529 701 904 | 0.030 |
| 2 115 181 256 744 777 | 0.030 |
| 18 395 466 555 721 727 | 0.030 |
| 204 480 542 682 721 753 | 0.030 |
| 72 179 247 594 685 841 | 0.030 |
| 42 122 217 270 822 899 | 0.030 |
| 359 379 475 553 571 905 | 0.030 |
| 115 268 284 487 830 900 | 0.024 |

Columns
Feature ID: feature index (see Table S3 for feature details)
MAE: mean absolute error between observed and calculated ctDNA fractions from 344 samples

TABLE S15
Information on predictive features for colorectal cancer

| ID | Transcript | Gene | Chr | Site | Region | Group | FPKM_{blood} | FPKMₜᵤₘₒᵣ |
|---|---|---|---|---|---|---|---|---|
| 2 | ENST00000027335.7 | CDH17 | 8 | 95220815 | promoter | tumor | 0.00 | 149.83 |
| 4 | ENST00000162330.9 | BCAR1 | 16 | 75285507 | promoter | tumor | 0.00 | 16.86 |
| 18 | ENST00000245451.8 | BMP4 | 14 | 54423529 | promoter | tumor | 0.00 | 16.43 |
| 32 | ENST00000264144.4 | LAMC2 | 1 | 183155423 | promoter | tumor | 0.01 | 31.46 |
| 35 | ENST00000267101.7 | ERBB3 | 12 | 56473645 | promoter | tumor | 0.00 | 40.98 |
| 39 | ENST00000270560.3 | TM4SF5 | 17 | 4675187 | promoter | tumor | 0.00 | 18.39 |
| 42 | ENST00000278937.6 | MPZL2 | 11 | 118134997 | promoter | tumor | 0.37 | 17.05 |
| 57 | ENST00000300119.7 | MYO1A | 12 | 57443901 | promoter | tumor | 0.00 | 15.11 |
| 59 | ENST00000300557.2 | PRR15L | 17 | 46035244 | promoter | tumor | 0.03 | 96.21 |
| 67 | ENST00000310836.10 | UGT8 | 4 | 115519611 | promoter | tumor | 0.04 | 14.08 |
| 70 | ENST00000311620.6 | ANKS4B | 16 | 21244986 | promoter | tumor | 0.00 | 10.61 |
| 72 | ENST00000317508.10 | PRSS8 | 16 | 31147083 | promoter | tumor | 0.11 | 143.22 |
| 75 | ENST00000318443.9 | CD276 | 15 | 73976554 | promoter | tumor | 0.00 | 11.45 |
| 78 | ENST00000322536.7 | DDX10 | 11 | 108535752 | promoter | tumor | 0.75 | 10.84 |
| 79 | ENST00000324038.6 | FAM101A | 12 | 124773710 | promoter | tumor | 0.00 | 10.08 |
| 82 | ENST00000326587.11 | MAGED1 | X | 51636698 | promoter | tumor | 0.60 | 16.14 |
| 84 | ENST00000331243.4 | FAM84A | 2 | 14772824 | promoter | tumor | 0.01 | 33.07 |
| 85 | ENST00000331595.8 | BGN | X | 152760397 | promoter | tumor | 0.60 | 74.92 |
| 86 | ENST00000332149.9 | TMPRSS2 | 21 | 42880086 | promoter | tumor | 0.02 | 43.57 |
| 88 | ENST00000334047.11 | F3 | 1 | 95007356 | promoter | tumor | 0.08 | 13.07 |
| 96 | ENST00000352551.9 | UBE2C | 20 | 44441313 | promoter | tumor | 0.00 | 10.99 |
| 109 | ENST00000358867.10 | TMEM126B | 11 | 85339629 | promoter | tumor | 0.47 | 17.68 |
| 115 | ENST00000361084.9 | RAB25 | 1 | 156030951 | promoter | tumor | 0.07 | 131.50 |
| 122 | ENST00000367284.9 | ELF3 | 1 | 201979645 | promoter | tumor | 0.02 | 50.55 |
| 124 | ENST00000367976.3 | CTGF | 6 | 132272513 | promoter | tumor | 0.32 | 44.18 |
| 126 | ENST00000368554.8 | PRAP1 | 10 | 135122914 | promoter | tumor | 0.00 | 32.41 |
| 139 | ENST00000374214.3 | UQCC2 | 6 | 33679504 | promoter | tumor | 1.00 | 17.77 |
| 153 | ENST00000380071.7 | RFC3 | 13 | 34392186 | promoter | tumor | 0.48 | 15.11 |
| 154 | ENST00000381134.7 | ARSE | X | 2882311 | promoter | tumor | 0.00 | 24.51 |
| 161 | ENST00000394201.8 | SCOC | 4 | 141294797 | promoter | tumor | 0.53 | 14.61 |
| 176 | ENST00000419308.6 | FOXA2 | 20 | 22565101 | promoter | tumor | 0.00 | 13.51 |
| 179 | ENST00000425042.6 | HID1 | 17 | 72968829 | promoter | tumor | 0.23 | 13.99 |
| 181 | ENST00000427250.5 | LSR | 19 | 35739905 | promoter | tumor | 0.00 | 13.66 |
| 197 | ENST00000463201.2 | PRAP1 | 10 | 135164879 | promoter | tumor | 0.00 | 54.46 |
| 202 | ENST00000473336.5 | RAB25 | 1 | 156031174 | promoter | tumor | 0.00 | 17.65 |
| 204 | ENST00000478194.1 | FERMT1 | 20 | 6074819 | promoter | tumor | 0.00 | 27.00 |
| 217 | ENST00000497734.5 | SRC | 20 | 35973088 | promoter | tumor | 0.00 | 12.44 |
| 230 | ENST00000532203.1 | MRPL17 | 11 | 6704547 | promoter | tumor | 0.65 | 21.73 |
| 247 | ENST00000554989.1 | CKB | 14 | 103987659 | promoter | tumor | 0.75 | 83.82 |
| 249 | ENST00000558580.1 | SORD | 15 | 45328434 | promoter | tumor | 0.95 | 15.73 |
| 250 | ENST00000561504.1 | ADIRF | 10 | 88728247 | promoter | tumor | 0.75 | 46.05 |
| 256 | ENST00000588605.5 | C19orf33 | 19 | 38794804 | promoter | tumor | 0.22 | 18.46 |
| 264 | ENST00000597153.5 | LGALS4 | 19 | 39303585 | promoter | tumor | 0.00 | 20.51 |
| 266 | ENST00000600324.5 | STAP2 | 19 | 4338818 | promoter | tumor | 0.02 | 13.85 |
| 268 | ENST00000605618.5 | LSR | 19 | 35739922 | promoter | tumor | 0.22 | 31.85 |
| 270 | ENST00000612794.1 | GPX2 | 14 | 65409623 | promoter | tumor | 0.00 | 23.73 |
| 275 | ENST00000619895.4 | TMC4 | 19 | 54676865 | promoter | tumor | 0.35 | 33.05 |
| 284 | ENST00000234347.9 | PRTN3 | 19 | 840960 | promoter | blood | 13.78 | 0.00 |
| 286 | ENST00000244709.8 | TREM1 | 6 | 41254457 | promoter | blood | 67.12 | 0.63 |
| 293 | ENST00000279452.10 | CD44 | 11 | 35198175 | promoter | blood | 10.76 | 0.00 |
| 322 | ENST00000355524.7 | FCAR | 19 | 55385736 | promoter | blood | 53.04 | 0.07 |
| 325 | ENST00000356864.3 | TNFRSF10C | 8 | 22960103 | promoter | blood | 108.86 | 0.56 |
| 332 | ENST00000367568.4 | STX11 | 6 | 144471663 | promoter | blood | 24.42 | 0.79 |
| 336 | ENST00000368737.4 | S100A12 | 1 | 153348125 | promoter | blood | 1099.02 | 0.31 |
| 337 | ENST00000371806.3 | FCN1 | 9 | 137809809 | promoter | blood | 379.64 | 0.88 |
| 348 | ENST00000380299.3 | HBD | 11 | 5255878 | promoter | blood | 138.84 | 0.07 |
| 359 | ENST00000398421.6 | NCF1 | 7 | 74188358 | promoter | blood | 11.04 | 0.04 |
| 364 | ENST00000413580.5 | PHOSPHO1 | 17 | 47308128 | promoter | blood | 42.76 | 0.05 |
| 379 | ENST00000454703.6 | ACSL1 | 4 | 185747273 | promoter | blood | 35.07 | 0.78 |
| 384 | ENST00000465984.5 | SLC11A1 | 2 | 219246911 | promoter | blood | 10.54 | 0.03 |
| 391 | ENST00000480395.5 | TRIM22 | 11 | 5717722 | promoter | blood | 17.36 | 0.70 |
| 395 | ENST00000485743.1 | HBB | 11 | 5248302 | promoter | blood | 128.52 | 0.00 |
| 407 | ENST00000496823.1 | BCL6 | 3 | 187463247 | promoter | blood | 21.33 | 0.20 |
| 410 | ENST00000509314.5 | FBXL5 | 4 | 15661487 | promoter | blood | 10.24 | 0.00 |
| 412 | ENST00000510784.6 | FAM65B | 6 | 25042396 | promoter | blood | 61.47 | 0.19 |
| 428 | ENST00000535669.6 | CD37 | 19 | 49838684 | promoter | blood | 33.70 | 0.19 |
| 429 | ENST00000539932.5 | SLC11A1 | 2 | 219246926 | promoter | blood | 50.32 | 0.02 |
| 447 | ENST00000576628.1 | ACAP1 | 17 | 7239916 | promoter | blood | 20.20 | 0.18 |
| 451 | ENST00000585901.6 | TYROBP | 19 | 36399149 | promoter | blood | 13.40 | 0.19 |
| 455 | ENST00000588673.3 | OAZ1 | 19 | 2270290 | promoter | blood | 24.28 | 0.69 |
| 464 | ENST00000595840.1 | LRRC25 | 19 | 18508421 | promoter | blood | 36.84 | 0.88 |
| 465 | ENST00000596426.5 | CD37 | 19 | 49838691 | promoter | blood | 55.55 | 0.57 |
| 466 | ENST00000596764.5 | VAV1 | 19 | 6772739 | promoter | blood | 12.25 | 0.59 |
| 467 | ENST00000597852.5 | CD37 | 19 | 49838675 | promoter | blood | 13.98 | 0.20 |
| 468 | ENST00000598034.5 | GMFG | 19 | 39826646 | promoter | blood | 43.77 | 0.45 |
| 469 | ENST00000599180.2 | FFAR2 | 19 | 35939203 | promoter | blood | 24.79 | 0.00 |
| 470 | ENST00000599716.5 | SHKBP1 | 19 | 41082793 | promoter | blood | 10.66 | 0.22 |
| 473 | ENST00000602185.5 | GMFG | 19 | 39826645 | promoter | blood | 14.09 | 0.00 |
| 475 | ENST00000605039.5 | BIN2 | 12 | 51717938 | promoter | blood | 11.02 | 0.11 |
| 477 | ENST00000612844.4 | FOLR3 | 11 | 71846756 | promoter | blood | 13.11 | 0.00 |
| 478 | ENST00000615439.4 | RASGRP4 | 19 | 38916945 | promoter | blood | 10.11 | 0.00 |
| 480 | ENST00000616356.4 | FCN1 | 9 | 137809723 | promoter | blood | 16.99 | 0.07 |
| 485 | ENST00000027335.7 | CDH17 | 8 | 95220711 | junction | tumor | 0.00 | 149.83 |
| 487 | ENST00000162330.9 | BCAR1 | 16 | 75285369 | junction | tumor | 0.00 | 16.86 |
| 511 | ENST00000262753.8 | POF1B | X | 84634644 | junction | tumor | 0.02 | 40.41 |
| 515 | ENST00000264144.4 | LAMC2 | 1 | 183155566 | junction | tumor | 0.01 | 31.46 |
| 522 | ENST00000270560.3 | TM4SF5 | 17 | 4675394 | junction | tumor | 0.00 | 18.39 |
| 525 | ENST00000278937.6 | MPZL2 | 11 | 118134811 | junction | tumor | 0.37 | 17.05 |
| 528 | ENST00000290913.7 | CHCHD6 | 3 | 126423242 | junction | tumor | 0.65 | 15.07 |
| 529 | ENST00000291525.11 | TFF3 | 21 | 43735403 | junction | tumor | 0.00 | 209.02 |
| 540 | ENST00000300119.7 | MYO1A | 12 | 57443671 | junction | tumor | 0.00 | 15.11 |
| 542 | ENST00000300557.2 | PRR15L | 17 | 46035023 | junction | tumor | 0.03 | 96.21 |
| 553 | ENST00000311620.6 | ANKS4B | 16 | 21245222 | junction | tumor | 0.00 | 10.61 |
| 555 | ENST00000317508.10 | PRSS8 | 16 | 31146735 | junction | tumor | 0.11 | 143.22 |
| 568 | ENST00000332149.9 | TMPRSS2 | 21 | 42880008 | junction | tumor | 0.02 | 43.57 |
| 571 | ENST00000334869.8 | LGMN | 14 | 93214834 | junction | tumor | 0.94 | 45.73 |
| 580 | ENST00000354900.7 | LSR | 19 | 35740034 | junction | tumor | 0.75 | 31.22 |
| 586 | ENST00000357166.10 | ZDHHC9 | X | 128977672 | junction | tumor | 0.29 | 20.11 |
| 590 | ENST00000358867.10 | TMEM126B | 11 | 85339732 | junction | tumor | 0.47 | 17.68 |
| 591 | ENST00000360325.11 | CLDN7 | 17 | 7165140 | junction | tumor | 0.09 | 133.65 |
| 594 | ENST00000360779.3 | SDCBP2 | 20 | 1309729 | junction | tumor | 0.00 | 17.84 |
| 595 | ENST00000361084.9 | RAB25 | 1 | 156031234 | junction | tumor | 0.07 | 131.50 |
| 604 | ENST00000367976.3 | CTGF | 6 | 132272247 | junction | tumor | 0.32 | 44.18 |
| 619 | ENST00000374214.3 | UQCC2 | 6 | 33679326 | junction | tumor | 1.00 | 17.77 |
| 629 | ENST00000379742.4 | POSTN | 13 | 38172745 | junction | tumor | 0.01 | 11.69 |
| 632 | ENST00000381134.7 | ARSE | X | 2882265 | junction | tumor | 0.00 | 24.51 |
| 634 | ENST00000389614.5 | GPX2 | 14 | 65409223 | junction | tumor | 0.10 | 652.10 |
| 639 | ENST00000394201.8 | SCOC | 4 | 141294871 | junction | tumor | 0.53 | 14.61 |
| 655 | ENST00000425042.6 | HID1 | 17 | 72968686 | junction | tumor | 0.23 | 13.99 |
| 656 | ENST00000425340.2 | FUT2 | 19 | 49199346 | junction | tumor | 0.02 | 13.10 |
| 673 | ENST00000472782.1 | ATP5G3 | 2 | 176046384 | junction | tumor | 0.62 | 10.87 |
| 675 | ENST00000478194.1 | FERMT1 | 20 | 6074721 | junction | tumor | 0.00 | 27.00 |
| 682 | ENST00000494801.5 | TCEAL4 | X | 102840552 | junction | tumor | 0.60 | 13.59 |
| 685 | ENST00000497734.5 | SRC | 20 | 35973290 | junction | tumor | 0.00 | 12.44 |
| 688 | ENST00000514985.5 | SEPP1 | 5 | 42811938 | junction | tumor | 0.59 | 49.95 |
| 695 | ENST00000528430.2 | PPP1R16A | 8 | 145726677 | junction | tumor | 0.92 | 22.33 |
| 701 | ENST00000542056.1 | GPRC5A | 12 | 13044598 | junction | tumor | 0.00 | 23.91 |
| 702 | ENST00000543445.5 | LDHA | 11 | 18416188 | junction | tumor | 0.00 | 71.47 |
| 703 | ENST00000543623.5 | PLCD3 | 17 | 43192462 | junction | tumor | 0.27 | 14.58 |
| 704 | ENST00000546314.5 | STARD10 | 11 | 72493311 | junction | tumor | 0.00 | 31.66 |
| 714 | ENST00000559087.5 | BMP4 | 14 | 54423477 | junction | tumor | 0.00 | 13.08 |
| 717 | ENST00000581920.1 | TYMS | 18 | 667752 | junction | tumor | 0.88 | 11.79 |
| 721 | ENST00000588605.5 | C19orf33 | 19 | 38794923 | junction | tumor | 0.22 | 18.46 |
| 727 | ENST00000597153.5 | LGALS4 | 19 | 39303482 | junction | tumor | 0.00 | 20.51 |
| 735 | ENST00000619895.4 | TMC4 | 19 | 54676734 | junction | tumor | 0.35 | 33.05 |
| 744 | ENST00000234347.9 | PRTN3 | 19 | 841069 | junction | blood | 13.78 | 0.00 |
| 748 | ENST00000246549.2 | FFAR2 | 19 | 35942667 | junction | blood | 58.85 | 0.34 |
| 749 | ENST00000246657.2 | CCR7 | 17 | 38721652 | junction | blood | 11.89 | 0.75 |
| 753 | ENST00000279452.10 | CD44 | 11 | 35198287 | junction | blood | 10.76 | 0.00 |
| 760 | ENST00000297239.10 | SYTL3 | 6 | 159082417 | junction | blood | 10.79 | 0.54 |
| 761 | ENST00000299663.7 | CLEC4E | 12 | 8693357 | junction | blood | 29.27 | 0.20 |
| 766 | ENST00000310544.8 | PHOSPHO1 | 17 | 47307830 | junction | blood | 30.92 | 0.08 |
| 777 | ENST00000343534.9 | C1orf162 | 1 | 112016652 | junction | blood | 37.68 | 0.74 |
| 780 | ENST00000354352.9 | SLC11A1 | 2 | 219247098 | junction | blood | 111.80 | 0.63 |
| 788 | ENST00000367025.7 | TRAF3IP3 | 1 | 209929654 | junction | blood | 17.52 | 0.64 |
| 790 | ENST00000367535.7 | NCF2 | 1 | 183559291 | junction | blood | 75.23 | 0.98 |
| 793 | ENST00000368015.1 | ARHGAP30 | 1 | 161039410 | junction | blood | 20.61 | 0.58 |
| 796 | ENST00000371806.3 | FCN1 | 9 | 137809615 | junction | blood | 379.64 | 0.88 |
| 822 | ENST00000422400.6 | VNN2 | 6 | 133078810 | junction | blood | 14.59 | 0.10 |
| 824 | ENST00000433194.6 | CDK5RAP2 | 9 | 123165594 | junction | blood | 22.55 | 0.52 |
| 830 | ENST00000454703.6 | ACSL1 | 4 | 185747070 | junction | blood | 35.07 | 0.78 |
| 839 | ENST00000480395.5 | TRIM22 | 11 | 5717885 | junction | blood | 17.36 | 0.70 |
| 841 | ENST00000483750.5 | WAS | X | 48542374 | junction | blood | 34.49 | 0.17 |
| 842 | ENST00000485743.1 | HBB | 11 | 5248160 | junction | blood | 128.52 | 0.00 |
| 852 | ENST00000496823.1 | BCL6 | 3 | 187463198 | junction | blood | 21.33 | 0.20 |
| 855 | ENST00000509314.5 | FBXL5 | 4 | 15661350 | junction | blood | 10.24 | 0.00 |
| 856 | ENST00000509339.1 | MXD3 | 5 | 176734782 | junction | blood | 30.07 | 0.08 |
| 858 | ENST00000513001.5 | ACSL1 | 4 | 185678973 | junction | blood | 17.65 | 0.10 |
| 867 | ENST00000540998.5 | CDC42SE1 | 1 | 151031955 | junction | blood | 10.52 | 0.00 |
| 874 | ENST00000553070.5 | NFE2 | 12 | 54694585 | junction | blood | 23.61 | 0.00 |
| 884 | ENST00000578402.5 | LIMD2 | 17 | 61777729 | junction | blood | 31.49 | 0.77 |
| 896 | ENST00000595840.1 | LRRC25 | 19 | 18508263 | junction | blood | 36.84 | 0.88 |
| 899 | ENST00000599180.2 | FFAR2 | 19 | 35939281 | junction | blood | 24.79 | 0.00 |
| 900 | ENST00000599716.5 | SHKBP1 | 19 | 41082891 | junction | blood | 10.66 | 0.22 |
| 901 | ENST00000600626.1 | C5AR2 | 19 | 47840426 | junction | blood | 12.99 | 0.11 |
| 903 | ENST00000605039.5 | BIN2 | 12 | 51717806 | junction | blood | 11.02 | 0.11 |
| 904 | ENST00000611028.2 | FOLR3 | 11 | 71846814 | junction | blood | 11.13 | 0.00 |
| 905 | ENST00000615439.4 | RASGRP4 | 19 | 38916709 | junction | blood | 10.11 | 0.00 |

Columns
ID: feature index, same as Table S3
Transcript: transcript ID
Gene: gene name
Chr: chromosome
Site: coordinate of nucleosome-depleted site (GRCh37)
Region: location of nucleosome-depleted site
Group: gene group based on its expression in blood and tumor
FPKM_{blood}: FPKM value in normal blood
FPKMₜᵤₘₒᵣ: FPKM value in tumor of colorectal cancer

TABLE S16
Information on additional predictive pan-cancer features

| Feature ID | MAE |
|---|---|
| 34 41 73 83 90 123 294 415 452 742 | 0.024 |
| 34 41 73 83 90 123 126 294 415 667 | 0.025 |
| 34 41 83 90 123 126 259 294 415 867 | 0.025 |
| 34 41 83 90 123 2 [symbol] 4 415 452 [symbol] 7 789 | 0.026 |
| 41 73 83 90 123 294 376 415 452 742 | 0.026 |
| 34 41 73 83 [symbol] 0 123 12 [symbol] 29 [symbol] 4 415 78 [symbol] | 0.026 |
| 34 64 135 201 27 [symbol] 415 653 735 772 778 | 0.026 |
| 41 73 83 90 123 12 [symbol] 294 37 [symbol] 415 452 | 0.026 |
| 34 52 201 415 653 6 [symbol] 735 7 [symbol] 3 772 77 [symbol] | 0.026 |
| 41 73 83 90 123 12 [symbol] 294 452 634 667 | 0.027 |
| 41 73 83 90 123 12 [symbol] 294 415 [symbol] 34 [symbol] 67 | 0.027 |
| 41 7 [symbol] 83 90 123 12 [symbol] 294 37 [symbol] 415 [symbol] 67 | 0.027 |
| 34 41 73 83 90 123 128 294 376 415 | 0.027 |
| 41 83 90 123 126 294 376 415 634 667 | 0.027 |
| 41 [symbol] 3 90 123 126 294 415 [symbol] 34 667 742 | 0.027 |
| 41 73 83 90 123 294 37 [symbol] 452 634 [symbol] 67 | 0.027 |
| 41 73 83 [symbol] 0 123 128 294 452 [symbol] 67 78 [symbol] | 0.028 |
| 41 73 83 [symbol] 0 123 12 [symbol] 294 452 742 78 [symbol] | 0.029 |
| 41 [symbol] 3 90 123 126 25 [symbol] 2 [symbol] 4 452 667 742 | 0.029 |
| 41 73 83 90 123 12 [symbol] 25 [symbol] 294 421 452 | 0.029 |
| 41 73 83 [symbol] 0 123 12 [symbol] 25 [symbol] 2 [symbol] 4 378 415 | 0.029 |
| 41 73 83 [symbol] 0 123 12 [symbol] 294 37 [symbol] 452 742 | 0.029 |
| 97 137 205 454 634 653 [symbol] 67 680 778 787 | 0.029 |
| 41 7 [symbol] 83 90 123 126 25 [symbol] 27 [symbol] 2 [symbol] 4 667 | 0.029 |
| 34 41 73 83 90 123 126 294 452 7 [symbol] | 0.030 |
| 34 41 73 83 90 123 259 294 452 667 | 0.030 |
| 34 41 73 83 90 123 126 259 294 452 | 0.030 |
| 34 41 73 83 90 123 126 294 376 452 | 0.030 |
| 41 83 90 12 [symbol] 259 294 415 [symbol] 34 667 742 | 0.030 |
| 41 83 90 123 2 [symbol] 415 [symbol] 34 [symbol] 7 742 789 | 0.030 |
| 41 73 83 90 123 294 376 452 [symbol] 7 789 | 0.030 |
| 41 73 83 90 123 12 [symbol] 259 294 376 452 | 0.030 |
| 41 73 83 90 123 259 294 37 [symbol] 634 [symbol] 7 | 0.030 |
| 34 41 73 83 90 123 126 135 2 [symbol] 4 376 | 0.030 |
| 97 137 201 23 [symbol] 278 348 415 454 772 778 | 0.031 |
| 41 73 83 90 123 128 294 37 [symbol] 452 789 | 0.031 |
| 41 73 83 [symbol] 0 123 128 249 37 [symbol] 634 667 | 0.031 |
| 41 73 83 [symbol] 0 123 12 [symbol] 2 [symbol] 4 376 421 742 | 0.031 |
| 34 41 73 83 90 123 12 [symbol] 294 376 667 | 0.031 |
| 34 41 83 [symbol] 0 123 12 [symbol] 2 [symbol] 4 37 [symbol] [symbol] 7 742 | 0.031 |
| [symbol] 34 8 [symbol] 135 278 428 621 [symbol] 80 7 [symbol] 8 78 [symbol] | 0.031 |
| 41 73 83 [symbol] 0 123 12 [symbol] 2 [symbol] 4 [symbol] 34 [symbol] 7 78 [symbol] | 0.032 |
| 73 [symbol] 3 90 123 126 13 [symbol] 259 294 376 452 | 0.032 |
| 34 13 [symbol] 236 278 428 [symbol] 21 [symbol] 0 7 [symbol] 3 7 [symbol] 78 [symbol] | 0.032 |
| [symbol] 2 137 236 258 288 304 338 388 674 778 | 0.032 |
| 41 73 83 [symbol] 0 123 12 [symbol] 2 [symbol] 4 [symbol] 34 667 742 | 0.032 |
| 34 137 201 23 [symbol] 242 278 415 735 772 778 | 0.032 |
| 41 7 [symbol] 83 [symbol] 0 123 12 [symbol] 2 [symbol] 4 37 [symbol] [symbol] 7 742 | 0.032 |
| 41 73 83 [symbol] 0 123 12 [symbol] 259 294 37 [symbol] [symbol] 67 | 0.032 |
| 41 73 83 [symbol] 0 123 126 294 6 [symbol] 7 742 789 | 0.032 |
| 34 89 135 205 242 428 621 680 768 789 | 0.032 |
| 34 41 73 83 [symbol] 0 123 12 [symbol] 294 667 742 | 0.032 |
| 34 41 73 83 [symbol] 0 123 12 [symbol] 294 [symbol] 67 7 [symbol] | 0.032 |
| 34 37 52 135 201 23 [symbol] 242 415 735 772 | 0.032 |
| 41 78 83 90 123 126 294 378 [symbol] 87 789 | 0.032 |
| 34 41 73 83 90 123 126 294 634 6 [symbol] 7 | 0.032 |
| 34 73 123 135 278 428 621 630 788 789 | 0.032 |
| 37 52 64 97 201 205 242 421 454 772 | 0.032 |
| 89 428 44 [symbol] 454 834 667 680 742 768 789 | 0.032 |
| 8 52 135 25 [symbol] 304 314 33 [symbol] 454 666 778 | 0.032 |
| 34 89 135 278 428 621 [symbol] 80 768 772 789 | 0.032 |
| 34 41 73 83 90 132 12 [symbol] 294 742 789 | 0.032 |
| 34 89 135 242 27 [symbol] 428 521 680 768 789 | 0.033 |
| 34 41 73 83 90 123 12 [symbol] 259 294 6 [symbol] 7 | 0.033 |
| 97 311 42 [symbol] 454 621 634 667 680 742 768 | 0.033 |
| 34 73 135 137 421 428 621 680 768 772 | 0.033 |
| 34 41 83 90 123 126 2 [symbol] 4 667 742 789 | 0.033 |
| 34 41 [symbol] 3 90 123 126 2 [symbol] 4 [symbol] 34 [symbol] 7 789 | 0.033 |
| 15 29 89 152 288 2 [symbol] 357 388 674 735 | 0.033 |
| 37 64 97 201 236 242 453 454 735 772 | 0.033 |
| 62 64 201 236 242 25 [symbol] 388 421 [symbol] 21 772 | 0.033 |
| 34 41 73 83 [symbol] 0 123 1 [symbol] 279 2 [symbol] 4 376 | 0.033 |
| 34 41 73 83 [symbol] 0 123 126 2 [symbol] 9 2 [symbol] 4 7 [symbol] | 0.033 |
| [symbol] 9 242 278 428 [symbol] 61 621 680 742 768 789 | 0.033 |
| 34 41 73 [symbol] 3 90 123 12 [symbol] 294 37 [symbol] 789 | 0.033 |
| 34 52 201 236 242 288 415 453 742 772 | 0.033 |
| 34 41 7 [symbol] 83 [symbol] 0 123 126 2 [symbol] 2 [symbol] 4 37 [symbol] | 0.033 |
| 52 258 259 304 357 388 402 44 [symbol] [symbol] 0 [symbol] 74 | 0.033 |
| [symbol] 34 89 135 238 242 428 742 788 789 | 0.033 |
| 41 83 [symbol] 0 12 [symbol] 126 2 [symbol] 9 294 634 6 [symbol] 7 789 | 0.033 |
| 34 41 83 90 12 [symbol] 126 294 376 742 789 | 0.033 |
| 9 52 201 28 [symbol] 357 388 45 [symbol] 547 630 674 | 0.033 |
| 8 152 259 2 [symbol] 9 309 435 544 735 775 778 | 0.033 |
| 73 137 415 421 428 454 621 634 [symbol] 7 [symbol] 0 | 0.033 |
| 9 37 [symbol] 4 201 236 258 415 674 735 778 | 0.034 |
| 41 73 83 [symbol] 0 123 294 6 [symbol] 4 [symbol] 7 742 78 [symbol] | 0.034 |
| 15 29 127 206 302 30 [symbol] 388 402 [symbol] 44 655 | 0.034 |
| 41 83 [symbol] 0 123 126 2 [symbol] 4 [symbol] 4 [symbol] 7 742 789 | 0.034 |
| 34 89 135 242 278 428 [symbol] 80 742 768 789 | 0.034 |
| 37 20 [symbol] 236 [symbol] 02 311 338 454 621 666 742 | 0.034 |
| 41 73 83 90 123 126 2 [symbol] 4 376 742 789 | 0.034 |
| 37 64 201 206 236 258 338 415 666 763 | 0.034 |
| 41 [symbol] 3 90 123 12 [symbol] 25 [symbol] 294 [symbol] 34 [symbol] 7 742 | 0.034 |
| 278 376 428 454 4 [symbol] 8 621 [symbol] 34 680 768 789 | 0.034 |
| 41 73 83 90 123 126 259 294 742 789 | 0.034 |
| 8 126 135 152 20 [symbol] 29 [symbol] 30 [symbol] 357 775 778 | 0.034 |
| [symbol] 242 311 428 454 634 6 [symbol] 0 742 768 789 | 0.034 |
| 9 52 135 242 259 357 388 3 [symbol] 2 43 [symbol] 745 | 0.034 |
| [symbol] 52 [symbol] 4 89 236 258 288 338 376 435 | 0.034 |
| 52 73 201 242 302 454 621 666 73 [symbol] 772 | 0.034 |
| 52 64 201 236 242 311 454 6 [symbol] 742 772 | 0.034 |
| 9 89 258 259 288 302 3 [symbol] 8 [symbol] 21 630 674 | 0.034 |
| 37 64 258 314 338 454 630 686 763 787 | 0.034 |
| 13 [symbol] 201 23 [symbol] 242 278 304 645 6 [symbol] 3 772 778 | 0.034 |
| [symbol] 9 135 137 20 [symbol] 311 428 4 [symbol] 4 680 7 [symbol] 8 789 | 0.034 |
| 73 137 278 421 42 [symbol] 454 [symbol] 21 634 [symbol] 0 768 | 0.034 |
| 52 [symbol] 4 137 20 [symbol] 311 428 454 6 [symbol] 0 788 789 | 0.034 |
| 34 137 415 421 428 [symbol] 21 634 [symbol] 57 [symbol] 80 7 [symbol] | 0.034 |
| 34 8 [symbol] 135 27 [symbol] 428 680 742 763 768 789 | 0.034 |
| 171 2 [symbol] 9 30 [symbol] 408 544 608 [symbol] 44 645 666 724 | 0.034 |
| 8 29 52 2 [symbol] 8 304 338 402 [symbol] 07 630 655 | 0.034 |
| 18 29 299 357 415 453 630 674 682 766 | 0.034 |
| 9 29 152 23 [symbol] 258 259 288 304 3 [symbol] 8 547 | 0.034 |
| 89 20 [symbol] 428 454 634 653 [symbol] 7 680 768 789 | 0.034 |
| 127 137 20 [symbol] 30 [symbol] 402 412 415 [symbol] 53 655 786 | 0.034 |
| 41 73 83 [symbol] 0 12 [symbol] 12 [symbol] 2 [symbol] 2 [symbol] 4 376 742 | 0.034 |
| 9 259 288 2 [symbol] 357 388 547 630 655 674 | 0.034 |
| [symbol] 4 137 284 304 621 634 [symbol] 45 6 [symbol] 3 772 778 | 0.034 |
| 152 357 38 [symbol] 415 435 544 674 735 768 778 | 0.034 |
| 34 73 97 135 278 421 428 763 768 772 | 0.034 |
| [symbol] 9 20 [symbol] 278 311 428 454 [symbol] 34 680 788 789 | 0.034 |
| [symbol] 34 97 137 201 236 242 735 772 778 | 0.035 |
| 89 152 201 206 288 338 37 [symbol] 392 [symbol] 5 775 | 0.035 |
| 37 201 23 [symbol] 242 304 376 621 634 [symbol] 67 742 | 0.035 |
| 29 52 89 152 259 288 299 309 357 388 | 0.035 |
| 34 89 135 137 278 428 621 680 768 772 | 0.035 |
| 8 52 30 [symbol] 435 544 607 [symbol] 30 735 775 778 | 0.035 |
| 8 29 52 236 357 388 435 4 [symbol] 3 454 674 | 0.035 |
| 52 152 205 2 [symbol] 8 388 630 735 745 778 787 | 0.035 |
| 73 135 242 278 421 428 454 621 [symbol] 0 768 | 0.035 |
| 29 89 135 152 258 331 482 547 [symbol] 34 742 | 0.035 |
| 52 174 331 338 435 454 4 [symbol] 544 [symbol] 21 634 | 0.035 |
| 34 135 137 242 275 415 428 621 680 7 [symbol] | 0.035 |
| 34 135 242 278 311 415 428 621 634 680 | 0.035 |
| 52 20 [symbol] 258 288 296 338 403 435 547 65 [symbol] | 0.035 |
| 205 278 376 42 [symbol] 454 621 [symbol] 34 [symbol] 80 768 788 | 0.035 |
| 208 376 42 [symbol] 454 621 634 [symbol] 67 6 [symbol] 0 768 789 | 0.035 |
| 34 89 205 278 428 621 634 [symbol] 0 7 [symbol] 789 | 0.035 |
| 15 29 170 171 2 [symbol] 9 402 6 [symbol] 8 [symbol] 82 766 787 | 0.035 |
| 34 89 135 23 [symbol] 278 311 428 44 [symbol] 768 789 | 0.035 |
| 170 2 [symbol] 2 296 412 435 60 [symbol] 644 703 708 7 [symbol] | 0.035 |
| 37 [symbol] 4 152 201 205 242 302 454 6 [symbol] 742 | 0.035 |
| 37 52 [symbol] 4 73 201 242 2 [symbol] 421 674 772 | 0.035 |
| 170 296 302 40 [symbol] 416 429 450 607 636 708 | 0.035 |
| 37 52 201 205 236 242 387 735 745 772 | 0.035 |
| [symbol] 37 52 64 135 242 304 621 742 772 | 0.035 |
| 34 89 135 137 205 27 [symbol] 428 763 7 [symbol] 789 | 0.035 |
| 52 64 126 201 205 288 415 454 742 772 | 0.035 |
| 137 201 242 259 288 421 454 [symbol] 21 [symbol] 80 772 | 0.035 |
| 89 135 20 [symbol] 236 25 [symbol] 338 357 435 547 778 | 0.035 |
| 29 52 201 25 [symbol] 357 38 [symbol] 402 454 544 807 | 0.035 |
| 8 37 [symbol] 4 236 258 304 338 3 [symbol] 7 376 5 [symbol] 4 | 0.036 |
| 29 127 306 3 [symbol] 402 412 544 607 6 [symbol] 3 [symbol] 55 | 0.036 |
| 37 97 2 [symbol] 242 278 311 454 [symbol] 34 735 772 | 0.036 |
| 15 89 152 206 259 28 [symbol] 357 388 482 724 | 0.036 |
| 34 152 201 242 258 25 [symbol] 288 2 [symbol] 547 6 [symbol] | 0.036 |
| 34 1 [symbol] 5 137 201 242 278 314 735 772 77 [symbol] | 0.036 |
| 152 171 30 [symbol] 402 544 60 [symbol] 653 655 766 775 | 0.036 |
| 89 135 428 621 634 667 680 742 768 78 [symbol] | 0.036 |
| 1 [symbol] 7 201 205 311 415 421 454 621 666 772 | 0.036 |
| 8 201 236 242 304 621 687 674 772 778 | 0.036 |
| 137 20 [symbol] 2 [symbol] 357 388 435 544 653 6 [symbol] 778 | 0.036 |
| [symbol] 37 [symbol] 4 201 205 206 259 3 [symbol] 7 630 735 | 0.036 |
| 37 52 [symbol] 4 201 242 338 421 4 [symbol] 4 735 772 | 0.036 |
| 73 278 421 428 454 [symbol] 21 634 680 742 7 [symbol] | 0.036 |
| 20 [symbol] 262 311 33 [symbol] 357 415 43 [symbol] 482 [symbol] 44 778 | 0.036 |
| 89 201 23 [symbol] 242 259 278 735 7 [symbol] 8 772 778 | 0.036 |
| 9 52 [symbol] 4 201 242 25 [symbol] 278 304 3 [symbol] 7 778 | 0.036 |
| 9 13 [symbol] 137 236 258 388 482 542 594 621 | 0.036 |
| 25 [symbol] 338 3 [symbol] 7 376 388 435 45 [symbol] 482 544 77 [symbol] | 0.036 |
| 9 29 288 298 2 [symbol] 388 402 4 [symbol] 3 630 775 | 0.036 |
| 211 306 37 [symbol] 408 4 [symbol] 0 544 [symbol] 45 655 708 724 | 0.036 |
| 89 135 428 454 634 [symbol] 67 680 742 768 789 | 0.036 |
| 137 311 428 454 498 621 [symbol] 34 [symbol] 67 680 742 | 0.036 |
| 29 127 308 357 376 402 435 544 [symbol] 55 76 [symbol] | 0.036 |
| 89 137 125 2 [symbol] 37 [symbol] 388 544 674 708 77 [symbol] | 0.036 |
| 205 376 428 454 4 [symbol] 8 621 [symbol] 34 [symbol] 67 680 768 | 0.036 |
| 37 [symbol] 4 259 338 376 454 [symbol] 4 [symbol] 30 [symbol] 6 763 | 0.036 |
| 37 84 201 20 [symbol] 2 [symbol] 311 454 [symbol] 6 742 772 | 0.036 |
| 8 52 135 201 27 [symbol] 367 376 7 [symbol] 772 778 | 0.036 |
| 13 [symbol] 205 278 428 454 621 [symbol] 34 6 [symbol] 0 7 [symbol] 7 [symbol] 9 | 0.036 |
| 137 242 278 376 428 454 [symbol] 34 6 [symbol] 0 768 78 [symbol] | 0.036 |
| 37 2 [symbol] 6 242 258 311 338 454 621 [symbol] 6 742 | 0.036 |
| 29 [symbol] 9 152 299 357 388 392 43 [symbol] 544 594 | 0.036 |
| 89 205 278 428 454 634 680 742 768 78 [symbol] | 0.036 |
| 34 52 201 20 [symbol] 236 242 415 653 735 772 | 0.036 |
| 9 29 135 152 258 304 388 542 594 630 | 0.036 |
| 89 201 288 357 37 [symbol] 388 392 402 6 [symbol] 775 | 0.036 |
| 41 73 83 90 123 259 294 687 742 789 | 0.036 |
| 127 29 [symbol] 306 357 388 435 544 608 6 [symbol] 775 | 0.036 |
| 8 29 64 89 152 205 288 338 454 68 [symbol] | 0.036 |
| 34 135 137 205 242 428 621 680 768 789 | 0.037 |
| 205 278 311 428 454 621 634 680 7 [symbol] 8 789 | 0.037 |
| 64 137 278 288 415 453 645 742 772 778 | 0.037 |
| 37 52 84 23 [symbol] 242 258 421 621 674 772 | 0.037 |
| 73 137 242 278 428 454 634 680 768 789 | 0.037 |
| 29 52 259 288 338 357 388 45 [symbol] 547 674 | 0.037 |
| 12 [symbol] 127 170 171 298 65 [symbol] [symbol] 66 724 766 77 [symbol] | 0.037 |
| 89 137 20 [symbol] 428 4 [symbol] 4 621 667 680 742 789 | 0.037 |
| 9 37 52 [symbol] 7 236 242 402 621 667 772 | 0.037 |
| 29 135 1 [symbol] 2 201 258 415 630 735 772 778 | 0.037 |
| 9 37 52 [symbol] 9 201 258 [symbol] 94 630 735 778 | 0.037 |
| 8 29 152 288 2 [symbol] 9 3 [symbol] 7 388 435 482 778 | 0.037 |
| 34 8 [symbol] 205 278 311 425 6 [symbol] 0 742 7 [symbol] 78 [symbol] | 0.037 |
| 37 52 64 201 205 236 311 348 742 772 | 0.037 |
| 29 2 [symbol] 6 2 [symbol] 9 304 3 [symbol] 8 402 412 453 [symbol] 6 735 | 0.037 |
| 37 64 201 205 415 594 630 6 [symbol] 735 78 [symbol] | 0.037 |
| 37 52 64 201 236 242 259 367 735 772 | 0.037 |
| 52 201 258 288 357 388 402 547 607 653 | 0.037 |
| 12 [symbol] 170 299 393 412 435 645 717 735 76 [symbol] | 0.037 |
| 152 206 296 41 [symbol] 435 4 [symbol] 0 63 [symbol] 6 [symbol] 708 76 [symbol] | 0.037 |
| 8 52 152 201 299 3 [symbol] 7 43 [symbol] 745 766 778 | 0.037 |
| 15 8 [symbol] 127 142 288 311 388 416 724 778 | 0.037 |
| 8 52 [symbol] 135 235 258 304 338 547 674 | 0.037 |
| 37 52 [symbol] 258 314 338 421 5 [symbol] 4 6 [symbol] 0 674 | 0.037 |
| 8 37 64 242 278 304 367 421 772 778 | 0.037 |
| 8 37 135 208 258 259 304 314 338 [symbol] 6 | 0.037 |
| 89 242 278 311 428 454 621 680 7 [symbol] 789 | 0.037 |
| 29 127 171 299 308 357 402 644 6 [symbol] 7 [symbol] | 0.037 |
| [symbol] 34 37 64 201 20 [symbol] 634 653 738 772 | 0.037 |
| 37 52 278 314 338 388 403 456 630 655 | 0.037 |
| 8 52 13 [symbol] 23 [symbol] 258 311 4 [symbol] 3 621 742 772 | 0.037 |
| [symbol] 152 206 306 376 435 544 607 708 766 | 0.037 |
| [symbol] 52 89 2 [symbol] 8 259 288 304 338 456 [symbol] 30 | 0.037 |
| 73 137 428 454 621 634 667 680 735 768 | 0.037 |
| 127 370 171 20 [symbol] 796 302 402 412 76 [symbol] 775 | 0.037 |
| 137 288 296 30 [symbol] 314 388 415 653 735 778 | 0.037 |
| 29 152 206 299 357 435 544 630 7 [symbol] 775 | 0.037 |
| 170 2 [symbol] 2 3 [symbol] 7 469 482 527 5 [symbol] 4 636 644 717 | 0.037 |
| 127 171 296 299 311 402 412 415 65 [symbol] 7 [symbol] | 0.037 |
| 8 37 52 [symbol] 4 201 338 480 547 686 674 | 0.037 |
| 34 135 137 278 37 [symbol] 428 634 650 768 772 | 0.037 |
| 20 [symbol] 211 302 306 408 490 [symbol] 45 6 [symbol] 724 763 | 0.037 |
| 52 152 232 264 388 415 490 674 735 775 | 0.037 |
| 37 [symbol] 2 64 242 339 453 4 [symbol] 634 7 [symbol] 772 | 0.037 |
| 170 171 299 418 44 [symbol] 585 594 653 736 76 [symbol] | 0.037 |
| 89 137 311 428 454 634 667 680 768 78 [symbol] | 0.037 |
| 34 135 205 278 421 428 621 634 6 [symbol] 0 768 | 0.037 |
| 152 201 206 242 288 357 388 392 655 775 | 0.037 |
| 152 209 296 308 314 357 402 544 7 [symbol] 6 775 | 0.037 |
| 8 52 64 89 258 259 288 338 453 742 | 0.037 |
| 9 84 242 278 304 367 421 547 772 77 [symbol] | 0.037 |
| 208 2 [symbol] 288 304 338 435 607 653 655 778 | 0.037 |
| 151 171 309 314 408 4 [symbol] 0 644 715 735 763 | 0.037 |
| 34 137 205 278 415 428 621 680 768 772 | 0.037 |
| 34 52 205 236 242 311 66 [symbol] 735 745 772 | 0.037 |
| 59 277 450 490 636 645 675 708 745 77 [symbol] | 0.038 |
| 34 37 84 89 25 [symbol] 288 338 453 821 674 | 0.038 |
| 40 211 231 277 30 [symbol] 314 402 403 45 [symbol] 636 | 0.038 |
| 135 205 242 278 428 454 621 680 768 78 [symbol] | 0.038 |
| 29 171 206 296 338 415 585 645 655 78 [symbol] | 0.038 |
| 29 152 302 388 392 453 454 544 6 [symbol] 703 | 0.038 |
| [symbol] 2 [symbol] 152 30 [symbol] 402 450 456 544 [symbol] 08 [symbol] 45 | 0.038 |
| 34 135 278 311 428 621 680 742 768 772 | 0.038 |
| 9 64 [symbol] 9 201 242 304 621 687 674 778 | 0.038 |
| 9 34 37 137 201 242 28 [symbol] 653 738 772 | 0.038 |
| 9 135 236 242 258 278 304 621 742 772 | 0.038 |
| 9 52 64 242 278 547 634 735 7 [symbol] 8 772 | 0.038 |
| 127 288 296 357 393 415 435 655 786 775 | 0.038 |
| [symbol] 52 64 278 367 547 634 653 735 772 | 0.038 |
| 8 37 52 [symbol] 4 201 23 [symbol] 258 745 772 778 | 0.038 |
| 152 201 402 415 498 653 674 735 745 76 [symbol] | 0.038 |
| 59 242 311 428 454 6 [symbol] 7 680 742 7 [symbol] 8 789 | 0.038 |
| 9 259 288 357 388 456 630 674 735 778 | 0.038 |
| 52 127 201 435 544 607 630 735 7 [symbol] 778 | 0.038 |
| 9 [symbol] 4 1 [symbol] 278 311 428 680 742 788 789 | 0.038 |
| 37 52 137 201 205 236 454 735 745 772 | 0.038 |
| 152 171 20 [symbol] 299 357 388 415 435 655 682 | 0.038 |
| 127 174 206 288 304 392 435 630 655 708 | 0.038 |
| 8 52 89 309 376 398 392 435 453 745 | 0.038 |
| 8 52 64 258 302 338 453 621 6 [symbol] 674 | 0.038 |
| 8 2 [symbol] 64 236 258 25 [symbol] 338 454 6 [symbol] 763 | 0.038 |
| 37 52 64 201 236 242 258 304 742 772 | 0.038 |
| 34 73 135 137 27 [symbol] 421 428 763 768 772 | 0.038 |
| 2 [symbol] 152 20 [symbol] 2 [symbol] 8 3 [symbol] 7 392 435 594 621 76 [symbol] | 0.038 |
| 127 171 208 296 415 435 544 [symbol] 5 766 775 | 0.038 |
| 73 171 206 277 298 314 544 558 65 [symbol] 775 | 0.038 |
| 9 170 408 421 527 542 544 644 [symbol] 45 775 | 0.038 |
| 8 37 64 206 258 259 338 454 630 753 | 0.038 |
| 137 278 311 428 454 [symbol] 21 634 658 742 7 [symbol] 8 | 0.038 |
| 8 9 64 135 201 338 435 630 748 77 [symbol] | 0.038 |
| 127 302 435 409 544 547 644 645 724 775 | 0.038 |
| 171 296 314 412 544 644 6 [symbol] 8 6 [symbol] 5 735 76 [symbol] | 0.038 |
| 15 152 211 231 232 305 415 708 724 766 | 0.038 |
| 171 28 [symbol] 357 408 4 [symbol] 0 544 645 653 724 776 | 0.038 |
| 37 52 64 201 205 236 242 454 735 772 | 0.038 |
| 34 73 137 205 278 428 634 680 768 789 | 0.038 |
| 52 64 205 236 258 387 376 621 674 772 | 0.038 |
| [symbol] 137 205 259 30 [symbol] 4 [symbol] 4 621 63 [symbol] [symbol] 80 735 | 0.038 |
| 137 205 278 378 415 428 621 634 6 [symbol] 0 768 | 0.038 |
| 89 137 152 20 [symbol] 236 258 [symbol] 288 309 435 453 | 0.038 |
| 170 171 412 415 453 4 [symbol] 2 594 6 [symbol] 682 76 [symbol] | 0.039 |
| 8 9 52 [symbol] 9 258 288 304 338 38 [symbol] 630 | 0.039 |
| 37 64 238 258 3 [symbol] 454 621 63 [symbol] 6 [symbol] 674 | 0.039 |
| 206 277 357 402 408 435 449 490 558 644 | 0.039 |
| 9 [symbol] 9 135 152 205 259 304 376 630 742 | 0.039 |
| [symbol] 37 52 84 258 311 314 338 454 6 [symbol] | 0.039 |
| [symbol] 52 89 126 258 28 [symbol] 30 [symbol] 338 454 630 | 0.039 |
| 15 206 302 306 402 412 636 645 653 735 | 0.039 |
| 15 89 137 152 288 296 357 3 [symbol] 8 415 674 | 0.039 |
| 15 40 127 171 206 408 415 544 594 708 | 0.039 |
| [symbol] 201 288 331 3 [symbol] 7 392 547 674 7 [symbol] 778 | 0.039 |
| 41 89 205 415 421 428 680 742 768 789 | 0.039 |
| 37 52 64 23 [symbol] 258 304 348 421 742 772 | 0.039 |
| 37 [symbol] 4 89 205 258 338 3 [symbol] 7 376 630 674 | 0.039 |
| 64 137 205 236 304 621 667 674 772 778 | 0.039 |
| 15 152 607 60 [symbol] 644 65 [symbol] 682 735 745 787 | 0.039 |
| 8 37 135 304 338 630 674 735 76 [symbol] 778 | 0.039 |
| 13 [symbol] 137 242 428 454 621 634 687 880 768 | 0.039 |
| 88 127 171 29 [symbol] 29 [symbol] 357 388 490 655 666 | 0.039 |
| 2 [symbol] 52 288 2 [symbol] 314 3 [symbol] 8 3 [symbol] 2 402 874 735 | 0.039 |
| 52 152 205 20 [symbol] 288 304 338 348 388 [symbol] 55 | 0.039 |
| 29 59 152 20 [symbol] 25 [symbol] 2 [symbol] 8 357 357 388 655 674 | 0.039 |
| 41 73 [symbol] 3 [symbol] 0 123 2 [symbol] 2 [symbol] 4 376 742 789 | 0.039 |
| 37 52 127 259 288 314 338 6 [symbol] 8 735 76 [symbol] | 0.039 |
| 137 205 278 311 42 [symbol] 454 634 680 768 7 [symbol] | 0.039 |
| 9 258 259 2 [symbol] 338 415 5 [symbol] 4 630 735 77 [symbol] | 0.039 |
| 8 [symbol] 2 [symbol] 8 304 335 388 392 547 [symbol] 55 674 778 | 0.039 |
| [symbol] 7 137 205 311 37 [symbol] 428 454 680 743 768 | 0.039 |
| 37 137 201 205 23 [symbol] 34 [symbol] 454 [symbol] 68 7 [symbol] 8 772 | 0.039 |
| [symbol] 37 52 64 201 242 27 [symbol] 304 772 77 [symbol] | 0.039 |
| 89 152 288 33 [symbol] 37 [symbol] 3 [symbol] 8 392 435 65 [symbol] 778 | 0.039 |
| 8 [symbol] 135 205 278 428 621 634 680 742 768 | 0.039 |
| 15 170 171 302 402 449 45 [symbol] 594 682 766 | 0.039 |
| 2 [symbol] 52 221 259 28 [symbol] 3 [symbol] 7 388 630 6 [symbol] 674 | 0.039 |
| 8 15 29 89 152 171 29 [symbol] 299 7 [symbol] 775 | 0.039 |
| 8 9 37 201 205 547 63 [symbol] 6 [symbol] 674 735 | 0.039 |
| 137 242 428 454 4 [symbol] 621 [symbol] 34 6 [symbol] 7 [symbol] 80 742 | 0.039 |
| [symbol] 35 135 242 278 428 621 680 7 [symbol] 8 772 | 0.039 |
| 8 52 152 288 304 306 357 388 453 490 | 0.039 |
| 201 236 258 259 28 [symbol] 482 547 674 735 778 | 0.039 |
| 170 171 232 296 29 [symbol] 456 585 708 724 76 [symbol] | 0.039 |
| 127 137 2 [symbol] 8 296 302 388 392 402 435 655 | 0.039 |
| 152 201 206 258 2 [symbol] 8 482 547 630 655 686 | 0.039 |
| 8 126 302 402 453 544 630 653 766 77 [symbol] | 0.039 |
| [symbol] 4 201 258 278 338 415 453 607 630 634 | 0.039 |
| 16 29 73 152 201 20 [symbol] 288 299 724 76 [symbol] | 0.039 |
| 64 8 [symbol] 236 258 278 304 653 742 772 778 | 0.039 |
| 8 9 304 357 435 544 653 [symbol] 742 778 | 0.039 |
| 89 135 137 242 428 454 680 742 768 778 | 0.039 |
| 89 135 205 25 [symbol] 428 454 680 742 768 778 | 0.039 |
| 152 20 [symbol] 25 [symbol] 258 306 309 314 357 435 607 | 0.039 |
| 127 171 20 [symbol] 252 408 415 450 544 644 76 [symbol] | 0.039 |
| 15 302 3 [symbol] 3 408 412 450 636 653 682 708 | 0.039 |
| 89 135 201 25 [symbol] 338 482 607 [symbol] 0 735 775 | 0.039 |
| 8 9 137 104 376 388 392 435 544 778 | 0.039 |
| 37 52 135 201 236 242 258 454 621 772 | 0.039 |
| 127 170 171 206 302 30 [symbol] 412 724 745 76 [symbol] | 0.040 |
| 8 9 8 [symbol] 135 242 278 367 821 674 772 | 0.040 |
| 9 34 137 205 37 [symbol] 428 634 667 680 768 | 0.040 |
| 8 89 137 152 288 338 357 415 [symbol] 74 778 | 0.040 |
| 34 137 205 278 428 680 763 768 772 7 [symbol] | 0.040 |
| 34 137 205 278 375 428 621 680 765 7 [symbol] | 0.040 |
| 8 64 258 401 454 634 666 667 674 787 | 0.040 |
| 8 37 [symbol] 9 151 205 206 258 304 [symbol] 874 | 0.040 |
| 137 205 278 428 454 621 634 680 7 [symbol] 8 789 | 0.040 |
| 2 [symbol] 2 3 [symbol] 7 527 574 585 619 675 70 [symbol] 775 787 | 0.040 |
| 97 137 311 428 454 634 667 680 742 7 [symbol] 8 | 0.040 |
| 38 213 367 40 [symbol] 527 558 619 628 682 787 | 0.040 |
| 37 [symbol] 2 64 20 [symbol] 236 242 2 [symbol] 9 367 735 772 | 0.040 |
| 206 259 277 296 306 402 412 645 [symbol] 55 724 | 0.040 |
| 206 211 262 2 [symbol] 412 630 644 674 708 76 [symbol] | 0.040 |
| 8 37 52 [symbol] 4 236 242 258 3 [symbol] 8 421 772 | 0.040 |
| 38 151 20 [symbol] 302 3 [symbol] 7 392 408 703 715 7 [symbol] 3 | 0.040 |
| 15 29 127 388 544 547 594 630 655 724 | 0.040 |
| 8 [symbol] 206 259 288 309 357 37 [symbol] 435 [symbol] 6 [symbol] | 0.040 |
| 34 37 201 236 242 278 311 735 772 778 | 0.040 |
| 127 171 20 [symbol] 304 306 482 544 653 686 7&6 | 0.040 |
| 52 64 201 236 242 304 421 621 674 772 | 0.040 |
| 304 30 [symbol] 393 406 435 608 645 703 708 724 | 0.040 |
| 37 64 201 205 242 259 311 454 742 772 | 0.040 |
| 137 205 278 37 [symbol] 428 498 821 634 680 788 | 0.040 |
| 127 231 314 357 402 544 644 682 7 [symbol] 77 [symbol] | 0.040 |
| 15 127 232 302 306 311 402 682 724 7 [symbol] | 0.040 |
| 8 29 288 304 38 [symbol] 392 435 630 655 778 | 0.040 |
| 2 [symbol] 298 30 [symbol] 314 388 402 415 435 544 653 | 0.040 |
| 8 127 171 2 [symbol] 309 490 544 655 708 775 | 0.040 |
| 9 34 137 278 378 428 621 6 [symbol] 0 7 [symbol] 8 78 [symbol] | 0.040 |
| 151 174 232 3 [symbol] 7 408 54 [symbol] 644 682 715 763 | 0.040 |
| 127 20 [symbol] 302 402 412 415 435 653 [symbol] 5 78 [symbol] | 0.040 |
| 8 52 89 258 288 304 338 357 630 674 | 0.040 |
| 89 171 2 [symbol] 8 338 357 376 392 449 453 655 | 0.040 |
| 37 64 236 28 [symbol] 338 454 4 [symbol] 0 66 [symbol] 670 674 | 0.040 |
| 221 259 288 308 388 402 435 450 655 735 | 0.040 |
| 11 41 83 90 123 135 294 415 454 [symbol] 34 | 0.029 |

Columns
Feature ID: feature index (see Table S10 for feature details)
MAE: mean absolute error between observed and calculated ctDNA fractions from 652 samples [symbol]
indicates data missing or illegible when filed

TABLE S17
Information on predictive pan-cancer features

| ID | Transcript | Gene | Chr | Site | Region | Group | FPKM_{blood} |
|---|---|---|---|---|---|---|---|
| 8 | ENST00000221954.6 | CEACAM4 | 19 | 42133442 | promoter | blood | 32.81 |
| 9 | ENST00000225275.3 | MPO | 17 | 56358296 | promoter | blood | 7.23 |
| 11 | ENST00000234347.9 | PRTN3 | 19 | 840960 | promoter | blood | 13.78 |
| 15 | ENST00000262407.5 | ITGA2B | 17 | 42466873 | promoter | blood | 12.34 |
| 29 | ENST00000299663.7 | CLEC4E | 12 | 8693559 | promoter | blood | 29.27 |
| 34 | ENST00000307395.4 | GP9 | 3 | 128779610 | promoter | blood | 9.72 |
| 37 | ENST00000312156.8 | NFE2 | 12 | 54689544 | promoter | blood | 11.15 |
| 38 | ENST00000314412.6 | FUT7 | 9 | 139927462 | promoter | blood | 16.90 |
| 39 | ENST00000314446.9 | LILRB2 | 19 | 54785039 | promoter | blood | 7.87 |
| 40 | ENST00000318507.6 | CXCR2 | 2 | 218990727 | promoter | blood | 157.27 |
| 41 | ENST00000324134.10 | NLRP12 | 19 | 54327597 | promoter | blood | 9.17 |
| 52 | ENST00000342063.4 | C19orf35 | 19 | 2282175 | promoter | blood | 8.64 |
| 64 | ENST00000375448.4 | PADI4 | 1 | 17634692 | promoter | blood | 95.04 |
| 73 | ENST00000380299.3 | HBD | 11 | 5255878 | promoter | blood | 138.84 |
| 83 | ENST00000392841.1 | HMBS | 11 | 118958697 | promoter | blood | 7.28 |
| 89 | ENST00000398421.6 | NCF1 | 7 | 74188358 | promoter | blood | 11.04 |
| 90 | ENST00000398632.3 | MX2 | 21 | 42774561 | promoter | blood | 7.28 |
| 97 | ENST00000413580.5 | PHOSPHO1 | 17 | 47308128 | promoter | blood | 42.76 |
| 123 | ENST00000460208.1 | LILRB3 | 19 | 54721567 | promoter | blood | 51.15 |
| 126 | ENST00000462275.5 | TBXAS1 | 7 | 139529072 | promoter | blood | 6.07 |
| 127 | ENST00000462927.5 | CYTH4 | 22 | 37678566 | promoter | blood | 26.95 |
| 135 | ENST00000465984.5 | SLC11A1 | 2 | 219246911 | promoter | blood | 10.54 |
| 137 | ENST00000466151.1 | PADI2 | 1 | 17409921 | promoter | blood | 7.13 |
| 142 | ENST00000467972.5 | SLC12A9 | 7 | 100454416 | promoter | blood | 6.80 |
| 151 | ENST00000469799.5 | SLC11A1 | 2 | 219247002 | promoter | blood | 8.27 |
| 152 | ENST00000469886.5 | CYTH4 | 22 | 37678556 | promoter | blood | 6.16 |
| 170 | ENST00000478902.1 | ZDHHC18 | 1 | 27177620 | promoter | blood | 12.52 |
| 171 | ENST00000479534.5 | PADI2 | 1 | 17401846 | promoter | blood | 5.78 |
| 174 | ENST00000480825.6 | CSF3R | 1 | 36941928 | promoter | blood | 127.36 |
| 201 | ENST00000487761.5 | GPSM3 | 6 | 32160645 | promoter | blood | 14.37 |
| 205 | ENST00000488970.1 | BTK | X | 100609209 | promoter | blood | 7.40 |
| 206 | ENST00000489175.1 | KCNE1 | 21 | 35884505 | promoter | blood | 6.75 |
| 211 | ENST00000490872.1 | SLC11A1 | 2 | 219254688 | promoter | blood | 26.15 |
| 213 | ENST00000492413.5 | SLC11A1 | 2 | 219247010 | promoter | blood | 17.89 |
| 221 | ENST00000495406.1 | CDK5RAP2 | 9 | 123152555 | promoter | blood | 10.53 |
| 231 | ENST00000500323.2 | DOK3 | 5 | 176935878 | promoter | blood | 10.88 |
| 232 | ENST00000502380.1 | DOK3 | 5 | 176937383 | promoter | blood | 8.44 |
| 236 | ENST00000509314.5 | FBXL5 | 4 | 15661487 | promoter | blood | 10.24 |
| 242 | ENST00000517813.1 | DENND3 | 8 | 142194587 | promoter | blood | 13.47 |
| 258 | ENST00000523369.1 | LCP2 | 5 | 169685502 | promoter | blood | 9.88 |
| 259 | ENST00000523530.1 | DENND3 | 8 | 142201373 | promoter | blood | 7.23 |
| 262 | ENST00000526387.5 | TBC1D10C | 11 | 67171386 | promoter | blood | 7.10 |
| 264 | ENST00000526980.5 | CSF3R | 1 | 36948500 | promoter | blood | 184.80 |
| 277 | ENST00000532897.5 | MKNK1 | 1 | 47037972 | promoter | blood | 6.23 |
| 278 | ENST00000533815.2 | BTNL8 | 5 | 180336564 | promoter | blood | 5.72 |
| 279 | ENST00000533968.1 | SPI1 | 11 | 47400038 | promoter | blood | 12.90 |
| 288 | ENST00000539932.5 | SLC11A1 | 2 | 219246926 | promoter | blood | 50.32 |
| 294 | ENST00000542481.1 | ATG16L2 | 11 | 72534940 | promoter | blood | 25.18 |
| 296 | ENST00000543576.5 | DENND1C | 19 | 6481798 | promoter | blood | 12.06 |
| 299 | ENST00000546200.5 | ARHGAP9 | 12 | 57871634 | promoter | blood | 66.03 |
| 302 | ENST00000551000.1 | ARHGAP9 | 12 | 57871650 | promoter | blood | 8.28 |
| 304 | ENST00000553070.5 | NFE2 | 12 | 54694799 | promoter | blood | 23.61 |
| 306 | ENST00000554736.5 | GNG2 | 14 | 52328042 | promoter | blood | 9.30 |
| 309 | ENST00000558332.3 | IL16 | 15 | 81591757 | promoter | blood | 7.66 |
| 311 | ENST00000559341.5 | MAN2A2 | 15 | 91459225 | promoter | blood | 8.61 |
| 314 | ENST00000560377.5 | PSTPIP1 | 15 | 77287726 | promoter | blood | 8.38 |
| 331 | ENST00000566082.1 | USB1 | 16 | 58049017 | promoter | blood | 6.82 |
| 338 | ENST00000570439.1 | ACAP1 | 17 | 7248650 | promoter | blood | 5.27 |
| 348 | ENST00000574548.1 | RNF167 | 17 | 4847675 | promoter | blood | 6.40 |
| 357 | ENST00000581974.1 | DHRS13 | 17 | 27226541 | promoter | blood | 5.97 |
| 367 | ENST00000587287.1 | RASGRP4 | 19 | 38902036 | promoter | blood | 6.80 |
| 376 | ENST00000590974.1 | LYL1 | 19 | 13213975 | promoter | blood | 5.48 |
| 388 | ENST00000597611.7 | FKBP8 | 19 | 18654887 | promoter | blood | 9.25 |
| 392 | ENST00000598529.5 | MYO1F | 19 | 8619448 | promoter | blood | 16.08 |
| 393 | ENST00000598907.5 | SHKBP1 | 19 | 41084102 | promoter | blood | 14.71 |
| 402 | ENST00000601502.1 | MYO1F | 19 | 8610602 | promoter | blood | 8.05 |
| 403 | ENST00000602101.6 | RASAL3 | 19 | 15575325 | promoter | blood | 7.16 |
| 408 | ENST00000605039.5 | BIN2 | 12 | 51717938 | promoter | blood | 11.02 |
| 412 | ENST00000612844.4 | FOLR3 | 11 | 71846756 | promoter | blood | 13.11 |
| 415 | ENST00000615340.4 | RASGRP4 | 19 | 38916837 | promoter | blood | 6.88 |
| 416 | ENST00000616356.4 | FCN1 | 9 | 137809723 | promoter | blood | 16.99 |
| 421 | ENST00000008938.4 | PGLYRP1 | 19 | 46525993 | junction | blood | 148.76 |
| 428 | ENST00000221954.6 | CEACAM4 | 19 | 42133268 | junction | blood | 32.81 |
| 429 | ENST00000225275.3 | MPO | 17 | 56357966 | junction | blood | 7.23 |
| 435 | ENST00000262407.5 | ITGA2B | 17 | 42466654 | junction | blood | 12.34 |
| 449 | ENST00000299663.7 | CLEC4E | 12 | 8693357 | junction | blood | 29.27 |
| 450 | ENST00000299665.2 | CLEC4D | 12 | 8666356 | junction | blood | 10.47 |
| 452 | ENST00000304076.6 | VAV1 | 19 | 6773022 | junction | blood | 7.61 |
| 453 | ENST00000304361.8 | CLEC12A | 12 | 10124286 | junction | blood | 10.23 |
| 454 | ENST00000307395.4 | GP9 | 3 | 128779693 | junction | blood | 9.72 |
| 456 | ENST00000310544.8 | PHOSPHO1 | 17 | 47307830 | junction | blood | 30.92 |
| 469 | ENST00000338372.6 | VSTM1 | 19 | 54566998 | junction | blood | 10.71 |
| 482 | ENST00000375448.4 | PADI4 | 1 | 17634809 | junction | blood | 95.04 |
| 490 | ENST00000380299.3 | HBD | 11 | 5255572 | junction | blood | 138.84 |
| 498 | ENST00000392841.1 | HMBS | 11 | 118958788 | junction | blood | 7.28 |
| 527 | ENST00000448367.5 | FES | 15 | 91432866 | junction | blood | 6.30 |
| 542 | ENST00000464994.5 | IL1R2 | 2 | 102608495 | junction | blood | 12.24 |
| 544 | ENST00000465984.5 | SLC11A1 | 2 | 219247098 | junction | blood | 10.54 |
| 546 | ENST00000466151.1 | PADI2 | 1 | 17409731 | junction | blood | 7.13 |
| 547 | ENST00000466612.5 | ABTB1 | 3 | 127392451 | junction | blood | 6.18 |
| 558 | ENST00000469532.1 | RAC2 | 22 | 37640154 | junction | blood | 9.83 |
| 561 | ENST00000471836.1 | PARVG | 22 | 44577797 | junction | blood | 6.53 |
| 574 | ENST00000478902.1 | ZDHHC18 | 1 | 27177722 | junction | blood | 12.52 |
| 585 | ENST00000483665.6 | FCGR2A | 1 | 161475342 | junction | blood | 6.60 |
| 594 | ENST00000485991.5 | CFP | X | 47489168 | junction | blood | 5.10 |
| 607 | ENST00000488970.1 | BTK | X | 100608858 | junction | blood | 7.40 |
| 608 | ENST00000489175.1 | KCNE1 | 21 | 35884325 | junction | blood | 6.75 |
| 619 | ENST00000495406.1 | CDK5RAP2 | 9 | 123152019 | junction | blood | 10.53 |
| 621 | ENST00000496915.1 | PREX1 | 20 | 47258945 | junction | blood | 5.23 |
| 628 | ENST00000504910.1 | HK3 | 5 | 176318389 | junction | blood | 6.36 |
| 630 | ENST00000509314.5 | FBXL5 | 4 | 15661350 | junction | blood | 10.24 |
| 634 | ENST00000513001.5 | ACSL1 | 4 | 185678973 | junction | blood | 17.65 |
| 636 | ENST00000517813.1 | DENND3 | 8 | 142195377 | junction | blood | 13.47 |
| 644 | ENST00000520887.1 | FAM49B | 8 | 130859050 | junction | blood | 7.15 |
| 645 | ENST00000521442.1 | ATP6V1B2 | 8 | 20075793 | junction | blood | 8.33 |
| 653 | ENST00000526387.5 | TBC1D10C | 11 | 67171411 | junction | blood | 7.10 |
| 655 | ENST00000527031.5 | JAK3 | 19 | 17958755 | junction | blood | 6.17 |
| 666 | ENST00000532897.5 | MKNK1 | 1 | 47037735 | junction | blood | 6.23 |
| 667 | ENST00000533815.2 | BTNL8 | 5 | 180336697 | junction | blood | 5.72 |
| 670 | ENST00000534754.5 | SORL1 | 11 | 121483541 | junction | blood | 9.84 |
| 674 | ENST00000538842.1 | ATG16L2 | 11 | 72527872 | junction | blood | 6.35 |
| 675 | ENST00000539134.1 | RELT | 11 | 73104962 | junction | blood | 6.58 |
| 680 | ENST00000542481.1 | ATG16L2 | 11 | 72535167 | junction | blood | 25.18 |
| 682 | ENST00000543576.5 | DENND1C | 19 | 6481690 | junction | blood | 12.06 |
| 703 | ENST00000564072.1 | SLCO3A1 | 15 | 92706776 | junction | blood | 8.74 |
| 708 | ENST00000564894.1 | RNF166 | 16 | 88767670 | junction | blood | 7.90 |
| 715 | ENST00000567038.1 | XPO6 | 16 | 28113168 | junction | blood | 8.97 |
| 717 | ENST00000568760.5 | DEF8 | 16 | 90024068 | junction | blood | 7.24 |
| 724 | ENST00000571303.1 | MLKL | 16 | 74725175 | junction | blood | 5.93 |
| 735 | ENST00000581287.5 | SLC16A3 | 17 | 80194107 | junction | blood | 11.97 |
| 736 | ENST00000581974.1 | DHRS13 | 17 | 27226145 | junction | blood | 5.97 |
| 742 | ENST00000585852.5 | FMNL1 | 17 | 43308054 | junction | blood | 19.81 |
| 745 | ENST00000587265.1 | CA4 | 17 | 58235488 | junction | blood | 5.45 |
| 763 | ENST00000595725.5 | CD37 | 19 | 49838866 | junction | blood | 20.04 |
| 766 | ENST00000597611.7 | FKBP8 | 19 | 18654719 | junction | blood | 9.25 |
| 768 | ENST00000598201.5 | SHKBP1 | 19 | 41086391 | junction | blood | 12.36 |
| 772 | ENST00000599180.2 | FFAR2 | 19 | 35939281 | junction | blood | 24.79 |
| 775 | ENST00000599716.5 | SHKBP1 | 19 | 41082891 | junction | blood | 10.66 |
| 776 | ENST00000600463.1 | IFI30 | 19 | 18286032 | junction | blood | 6.41 |
| 778 | ENST00000601502.1 | MYO1F | 19 | 8610534 | junction | blood | 8.05 |
| 787 | ENST00000614135.4 | RASGRP4 | 19 | 38916709 | junction | blood | 8.18 |
| 789 | ENST00000616356.4 | FCN1 | 9 | 137809615 | junction | blood | 16.99 |
| 666 | ENST00000532897.5 | MKNK1 | 1 | 47037735 | junction | blood | 6.23 |
| 667 | ENST00000533815.2 | BTNL8 | 5 | 180336697 | junction | blood | 5.72 |
| 674 | ENST00000538842.1 | ATG16L2 | 11 | 72527872 | junction | blood | 6.35 |
| 680 | ENST00000542481.1 | ATG16L2 | 11 | 72535167 | junction | blood | 25.18 |
| 695 | ENST00000559341.5 | MAN2A2 | 15 | 91459486 | junction | blood | 8.61 |
| 708 | ENST00000564894.1 | RNF166 | 16 | 88767670 | junction | blood | 7.9 |
| 736 | ENST00000581974.1 | DHRS13 | 17 | 27226145 | junction | blood | 5.97 |
| 742 | ENST00000585852.5 | FMNL1 | 17 | 43308054 | junction | blood | 19.81 |
| 766 | ENST00000597611.7 | FKBP8 | 19 | 18654719 | junction | blood | 9.25 |
| 772 | ENST00000599180.2 | FFAR2 | 19 | 35939281 | junction | blood | 24.79 |
| 775 | ENST00000599716.5 | SHKBP1 | 19 | 41082891 | junction | blood | 10.66 |
| 776 | ENST00000600463.1 | IFI30 | 19 | 18286032 | junction | blood | 6.41 |
| 787 | ENST00000614135.4 | RASGRP4 | 19 | 38916709 | junction | blood | 8.18 |
| 789 | ENST00000616356.4 | FCN1 | 9 | 137809615 | junction | blood | 16.99 |

Columns
ID: feature index
Transcript: transcript ID
Gene: gene name
Chr: chromosome
Site: coordinate of nucleosome-depleted site (GRCh37)
Region: location of nucleosome-depleted site
Group: gene group based on its expression in blood and tumor
FPKM_{blood}: FPKM value in normal blood. Their FPKM values in tumors of 20 cancer types are all <1.

TABLE S18
All predictive feature combinations for CRC using in silico
samples generated with random subsets of healthy samples.

| Feature ID | MAE |
|---|---|
| 364 379 475 788 730 905 | 0.030 |
| 284 384 475 788 790 906 | 0.031 |
| 284 364 379 475 758 905 | 0.031 |
| 284 364 379 475 790 906 | 0.032 |
| 179 407 788 830 900 905 | 0.032 |
| 364 379 760 790 852 903 | 0.033 |
| 39 540 760 788 830 900 | 0.033 |
| 35 39 410 540 760 830 | 0.033 |
| 284 364 475 760 788 905 | 0.033 |
| 364 540 760 830 903 905 | 0.033 |
| 364 379 475 703 788 905 | 0.033 |
| 284 364 475 703 788 905 | 0.033 |
| 364 475 760 788 790 905 | 0.034 |
| 35 476 673 830 852 890 | 0.034 |
| 364 379 540 760 790 903 | 0.034 |
| 364 379 475 703 760 790 | 0.034 |
| 364 379 407 760 790 903 | 0.034 |
| 39 125 379 478 852 903 | 0.034 |
| 39 364 475 788 790 905 | 0.034 |
| 364 379 475 788 900 905 | 0.034 |
| 39 379 410 540 703 760 | 0.034 |
| 39 379 407 540 760 790 | 0.034 |
| 364 475 540 788 790 905 | 0.034 |
| 364 760 790 830 900 903 | 0.035 |
| 410 478 540 688 760 830 | 0.035 |
| 478 760 788 830 900 903 | 0.035 |
| 379 407 475 788 790 905 | 0.035 |
| 364 475 703 788 790 905 | 0.035 |
| 39 364 475 703 788 905 | 0.035 |
| 364 475 788 790 900 905 | 0.035 |
| 35 39 301 540 830 903 | 0.035 |
| 379 475 540 788 790 905 | 0.035 |
| 379 475 703 788 790 905 | 0.035 |
| 364 379 760 790 900 903 | 0.035 |
| 115 126 480 529 830 903 | 0.035 |
| 284 410 469 540 571 830 | 0.035 |
| 39 379 407 703 790 903 | 0.035 |
| 384 379 540 703 793 903 | 0.035 |
| 36 322 469 478 830 903 | 0.035 |
| 284 364 475 703 790 905 | 0.036 |
| 364 407 475 788 790 905 | 0.036 |
| 126 284 410 480 830 890 | 0.036 |
| 39 364 379 407 540 903 | 0.036 |
| 39 115 379 407 790 903 | 0.036 |
| 364 379 478 790 900 903 | 0.037 |
| 39 67 379 703 900 903 | 0.037 |
| 39 379 407 540 703 903 | 0.037 |
| 407 478 760 788 830 900 | 0.037 |
| 39 115 379 407 703 790 | 0.037 |
| 39 115 284 379 540 703 | 0.037 |
| 86 284 478 760 830 903 | 0.037 |
| 364 475 540 788 900 905 | 0.037 |
| 407 760 788 830 900 903 | 0.037 |
| 39 379 407 703 760 903 | 0.037 |
| 86 364 379 410 900 903 | 0.037 |
| 301 470 473 553 639 890 | 0.037 |
| 364 475 760 788 900 905 | 0.037 |
| 35 39 478 540 673 830 | 0.037 |
| 379 407 475 703 790 905 | 0.037 |
| 39 284 379 703 760 903 | 0.037 |
| 126 364 379 407 475 760 | 0.037 |
| 39 126 478 830 852 903 | 0.038 |
| 364 379 407 410 760 903 | 0.038 |
| 126 284 410 469 540 830 | 0.038 |
| 284 359 410 469 540 830 | 0.038 |
| 364 475 703 788 900 905 | 0.038 |
| 256 478 594 727 830 852 | 0.038 |
| 364 407 475 703 788 905 | 0.038 |
| 39 126 379 410 469 478 | 0.038 |
| 379 407 475 703 786 905 | 0.038 |
| 364 407 475 703 790 905 | 0.038 |
| 86 407 410 760 830 900 | 0.038 |
| 39 364 703 830 900 903 | 0.038 |
| 379 475 703 788 790 900 | 0.038 |
| 39 284 540 594 727 830 | 0.038 |
| 35 284 410 571 760 830 | 0.038 |
| 703 760 790 830 900 903 | 0.038 |
| 86 470 655 842 856 890 | 0.038 |
| 35 284 410 540 571 830 | 0.038 |
| 39 475 703 786 790 905 | 0.038 |
| 284 379 407 410 469 571 | 0.038 |
| 379 540 760 790 900 903 | 0.038 |
| 379 407 760 790 900 903 | 0.038 |
| 86 115 126 469 553 721 | 0.039 |
| 284 322 410 469 540 830 | 0.039 |
| 35 39 410 478 540 830 | 0.039 |
| 284 469 478 540 571 830 | 0.039 |
| 407 760 790 830 900 903 | 0.039 |
| 284 410 480 540 571 830 | 0.039 |
| 35 86 284 469 478 830 | 0.039 |
| 284 410 469 529 540 830 | 0.039 |
| 115 284 379 407 760 790 | 0.039 |
| 39 268 306 322 466 744 | 0.039 |
| 284 410 478 540 571 830 | 0.039 |
| 35 86 306 673 793 852 | 0.039 |
| 407 703 760 790 830 903 | 0.039 |
| 115 364 379 407 760 903 | 0.039 |
| 284 410 480 629 760 830 | 0.039 |
| 284 478 540 571 830 852 | 0.039 |
| 72 197 256 395 473 858 | 0.039 |
| 364 478 760 830 900 903 | 0.039 |
| 284 379 410 673 760 852 | 0.039 |
| 39 284 379 407 703 903 | 0.039 |
| 39 379 407 410 760 900 | 0.039 |
| 364 540 760 830 900 903 | 0.040 |
| 39 407 475 703 788 905 | 0.040 |
| 35 364 379 540 703 903 | 0.040 |
| 72 204 301 451 473 639 | 0.040 |
| 18 70 256 439 467 777 | 0.040 |
| 86 359 391 395 455 890 | 0.040 |
| 35 478 540 673 760 830 | 0.040 |
| 364 379 407 478 540 903 | 0.040 |
| 67 364 379 407 760 903 | 0.040 |
| 39 379 407 703 900 903 | 0.040 |
| 18 72 275 393 468 842 | 0.040 |
| 284 478 540 727 830 690 | 0.040 |
| 35 39 703 793 852 903 | 0.040 |
| 468 469 553 559 652 842 | 0.040 |
| 407 540 760 790 830 900 | 0.040 |

Columns
Feature ID: feature index (see Table S3 for feature details)
MAE: mean absolute error between observed and calculated ctDNA fractions from 344 samples

TABLE S19
Information on predictive features for CRC using in silico
samples generated with random subsets of healthy samples.

| ID | Transcript | Gene | Chr | Site | Region | Group | FPKM_{blood} | FPKMₜᵤₘₒᵣ |
|---|---|---|---|---|---|---|---|---|
| 18 | ENST00000245451.8 | BMP4 | 14 | 54423529 | promoter | tumor | 0 | 16.43 |
| 35 | ENST00000267101.7 | ERBB3 | 12 | 56473645 | promoter | tumor | 0 | 40.98 |
| 39 | ENST00000270560.3 | TM4SF5 | 17 | 4675187 | promoter | tumor | 0 | 18.39 |
| 67 | ENST00000310836.10 | UGT8 | 4 | 115519611 | promoter | tumor | 0.04 | 14.08 |
| 70 | ENST00000311620.6 | ANKS4B | 16 | 21244986 | promoter | tumor | 0 | 10.61 |
| 72 | ENST00000317508.10 | PRSS8 | 16 | 31147083 | promoter | tumor | 0.11 | 143.22 |
| 86 | ENST00000332149.9 | TMPRSS2 | 21 | 42880086 | promoter | tumor | 0.02 | 43.57 |
| 115 | ENST00000361084.9 | RAB25 | 1 | 156030951 | promoter | tumor | 0.07 | 131.5 |
| 126 | ENST00000368554.8 | PRAP1 | 10 | 135122914 | promoter | tumor | 0 | 32.41 |
| 179 | ENST00000425042.6 | HID1 | 17 | 72968829 | promoter | tumor | 0.23 | 13.99 |
| 197 | ENST00000463201.2 | PRAP1 | 10 | 135164879 | promoter | tumor | 0 | 54.46 |
| 204 | ENST00000478194.1 | FERMT1 | 20 | 6074819 | promoter | tumor | 0 | 27 |
| 256 | ENST00000588605.5 | C19orf33 | 19 | 38794804 | promoter | tumor | 0.22 | 18.46 |
| 268 | ENST00000605618.5 | LSR | 19 | 35739922 | promoter | tumor | 0.22 | 31.85 |
| 275 | ENST00000619895.4 | TMC4 | 19 | 54676865 | promoter | tumor | 0.35 | 33.05 |
| 284 | ENST00000234347.9 | PRTN3 | 19 | 840960 | promoter | blood | 13.78 | 0 |
| 301 | ENST00000299663.7 | CLEC4E | 12 | 8693559 | promoter | blood | 29.27 | 0.2 |
| 306 | ENST00000310544.8 | PHOSPHO1 | 17 | 47307890 | promoter | blood | 30.92 | 0.08 |
| 322 | ENST00000355524.7 | FCAR | 19 | 55385736 | promoter | blood | 53.04 | 0.07 |
| 359 | ENST00000398421.6 | NCF1 | 7 | 74188358 | promoter | blood | 11.04 | 0.04 |
| 364 | ENST00000413580.5 | PHOSPHO1 | 17 | 47308128 | promoter | blood | 42.76 | 0.05 |
| 379 | ENST00000454703.6 | ACSL1 | 4 | 185747273 | promoter | blood | 35.07 | 0.78 |
| 391 | ENST00000480395.5 | TRIM22 | 11 | 5717722 | promoter | blood | 17.36 | 0.7 |
| 393 | ENST00000483750.5 | WAS | X | 48542217 | promoter | blood | 34.49 | 0.17 |
| 395 | ENST00000485743.1 | HBB | 11 | 5248302 | promoter | blood | 128.52 | 0 |
| 407 | ENST00000496823.1 | BCL6 | 3 | 187463247 | promoter | blood | 21.33 | 0.2 |
| 410 | ENST00000509314.5 | FBXL5 | 4 | 15661487 | promoter | blood | 10.24 | 0 |
| 439 | ENST00000553070.5 | NFE2 | 12 | 54694799 | promoter | blood | 23.61 | 0 |
| 451 | ENST00000585901.6 | TYROBP | 19 | 36399149 | promoter | blood | 13.4 | 0.19 |
| 455 | ENST00000588673.3 | OAZ1 | 19 | 2270290 | promoter | blood | 24.28 | 0.69 |
| 466 | ENST00000596764.5 | VAV1 | 19 | 6772739 | promoter | blood | 12.25 | 0.59 |
| 467 | ENST00000597852.5 | CD37 | 19 | 49838675 | promoter | blood | 13.98 | 0.2 |
| 468 | ENST00000598034.5 | GMFG | 19 | 39826646 | promoter | blood | 43.77 | 0.45 |
| 469 | ENST00000599180.2 | FFAR2 | 19 | 35939203 | promoter | blood | 24.79 | 0 |
| 470 | ENST00000599716.5 | SHKBP1 | 19 | 41082793 | promoter | blood | 10.66 | 0.22 |
| 473 | ENST00000602185.5 | GMFG | 19 | 39826645 | promoter | blood | 14.09 | 0 |
| 475 | ENST00000605039.5 | BIN2 | 12 | 51717938 | promoter | blood | 11.02 | 0.11 |
| 478 | ENST00000615439.4 | RASGRP4 | 19 | 38916945 | promoter | blood | 10.11 | 0 |
| 480 | ENST00000616356.4 | FCN1 | 9 | 137809723 | promoter | blood | 16.99 | 0.07 |
| 529 | ENST00000291525.11 | TFF3 | 21 | 43735403 | junction | tumor | 0 | 209.02 |
| 540 | ENST00000300119.7 | MYO1A | 12 | 57443671 | junction | tumor | 0 | 15.11 |
| 553 | ENST00000311620.6 | ANKS4B | 16 | 21245222 | junction | tumor | 0 | 10.61 |
| 559 | ENST00000318683.6 | B3GNT3 | 19 | 17906015 | junction | tumor | 0 | 57.75 |
| 571 | ENST00000334869.8 | LGMN | 14 | 93214834 | junction | tumor | 0.94 | 45.73 |
| 594 | ENST00000360779.3 | SDCBP2 | 20 | 1309729 | junction | tumor | 0 | 17.84 |
| 639 | ENST00000394201.8 | SCOC | 4 | 141294871 | junction | tumor | 0.53 | 14.61 |
| 652 | ENST00000419308.6 | FOXA2 | 20 | 22564830 | junction | tumor | 0 | 13.51 |
| 655 | ENST00000425042.6 | HID1 | 17 | 72968686 | junction | tumor | 0.23 | 13.99 |
| 673 | ENST00000472782.1 | ATP5G3 | 2 | 176046384 | junction | tumor | 0.62 | 10.87 |
| 688 | ENST00000514985.5 | SEPP1 | 5 | 42811938 | junction | tumor | 0.59 | 49.95 |
| 703 | ENST00000543623.5 | PLCD3 | 17 | 43192462 | junction | tumor | 0.27 | 14.58 |
| 721 | ENST00000588605.5 | C19orf33 | 19 | 38794923 | junction | tumor | 0.22 | 18.46 |
| 727 | ENST00000597153.5 | LGALS4 | 19 | 39303482 | junction | tumor | 0 | 20.51 |
| 744 | ENST00000234347.9 | PRTN3 | 19 | 841069 | junction | blood | 13.78 | 0 |
| 760 | ENST00000297239.10 | SYTL3 | 6 | 159082417 | junction | blood | 10.79 | 0.54 |
| 777 | ENST00000343534.9 | C1orf162 | 1 | 112016652 | junction | blood | 37.68 | 0.74 |
| 788 | ENST00000367025.7 | TRAF3IP3 | 1 | 209929654 | junction | blood | 17.52 | 0.64 |
| 790 | ENST00000367535.7 | NCF2 | 1 | 183559291 | junction | blood | 75.23 | 0.98 |
| 793 | ENST00000368015.1 | ARHGAP30 | 1 | 161039410 | junction | blood | 20.61 | 0.58 |
| 830 | ENST00000454703.6 | ACSL1 | 4 | 185747070 | junction | blood | 35.07 | 0.78 |
| 842 | ENST00000485743.1 | HBB | 11 | 5248160 | junction | blood | 128.52 | 0 |
| 852 | ENST00000496823.1 | BCL6 | 3 | 187463198 | junction | blood | 21.33 | 0.2 |
| 856 | ENST00000509339.1 | MXD3 | 5 | 176734782 | junction | blood | 30.07 | 0.08 |
| 858 | ENST00000513001.5 | ACSL1 | 4 | 185678973 | junction | blood | 17.65 | 0.1 |
| 890 | ENST00000589900.5 | ICAM3 | 19 | 10450215 | junction | blood | 21.24 | 0 |
| 900 | ENST00000599716.5 | SHKBP1 | 19 | 41082891 | junction | blood | 10.66 | 0.22 |
| 903 | ENST00000605039.5 | BIN2 | 12 | 51717806 | junction | blood | 11.02 | 0.11 |
| 905 | ENST00000615439.4 | RASGRP4 | 19 | 38916709 | junction | blood | 10.11 | 0 |

Columns
ID: feature index, same as Table S3
Transcript: transcript ID
Gene: gene name
Chr: chromosome
Site: coordinate of nucleosome-depleted site (GRCh37)
Region: location of nucleosome-depleted site
Group: gene group based on its expression in blood and tumor
FPKM_{blood}: FPKM value in normal blood
FPKMₜᵤₘₒᵣ: FPKM value in tumor of colorectal cancer

TABLE S20
Columns
Feature ID: feature index (see Table S10 for feature details)
MAE: mean absolute error between observed and calculated ctDNA fractions from 652 samples
All predictive pan-cancer feature combinations using in silico samples
generated with random subsets of healthy samples.

| Feature ID | MAE | Feature ID | MAE |
|---|---|---|---|
| 34 41 90 126 294 376 388 415 742 772 | 0.034 | 29 201435 454667 630 742 766 772 787 | 0.041 |
| 34 41 90 126 294 376 388 415 500 772 | 0.034 | 34 41 288 490667 680 742 768 772 787 | 0.041 |
| 34 41 90 126 236 294 376 388 415 500 | 0.036 | 29 34 36 64 436 667 680 742 766 772 | 0.041 |
| 34 41 90 126 201 294 376 388 415 500 | 0.036 | 29 128 428 454 634 645 680 742 772 787 | 0.041 |
| 34 41 90 126 201 294 376 388 415 772 | 0.036 | 34 41 90 126 236 294 376 388 742 772 | 0.041 |
| 34 4 1 64 90 126 294 388 415 490 500 | 0.036 | 57 90 126 201 236 294 388 415 490 500 | 0.041 |
| 34 41 64 90 126 294 376 388 415 500 | 0.036 | 41 90126 201 236 294 376 388 456 500 | 0.041 |
| 34 41 90 1 26 201 294 388 500 742 772 | 0.037 | 34 4190126135 201 294 388 500 772 | 0.041 |
| 29 34 415 428 490 645 680 742 766 772 | 0.038 | 34 135 415 645667 680 742 766 772 775 | 0.041 |
| 34 41 90 126 201 230 294 376 388 500 | 0.038 | 34 41 90126135 201 294 388 490 500 | 0.041 |
| 29 34 36 64 428 490 742 766 772 775 | 0.038 | 29 36 396 415 428 435 454 742 766 789 | 0.041 |
| 34 41 64 90 126 201 294 388 742 772 | 0.038 | 29 135 428 454 667 680 766 772 775 789 | 0.042 |
| 41 90 126 201 236 294 376 388 415 500 | 0.038 | 34 41 64 90 126 135 201294 388 500 | 0.042 |
| 29 57 64 126 428 454 742 766 772 789 | 0.038 | 34 41 64 90 126 201 294 376 388 772 | 0.042 |
| 34 41 90 126 201 236 294 388 490 500 | 0.039 | 34 41 90 126 236 294 376 388 500 772 | 0.042 |
| 34 126 135 428 680 742 766 772 787 789 | 0.039 | 41 84 90126 201 236 294 378 388 500 | 0.042 |
| 34 41 90 126 201 294 388 500 772 775 | 0.039 | 13 29 396 415 428 435454 880 786 789 | 0.042 |
| 34 41 90 1 26 201 236 294 376 388 772 | 0.039 | 29 34 135 311 428 680 742 766 772 775 | 0.042 |
| 29 34 80135 415 428 742 766 772 775 | 0.039 | 41 90 126 201 236 294 378 388 490 500 | 0.042 |
| 34 41 90 201 236 294 376 388 500 772 | 0.033 | 64 90 126135 201 294 376 388 415 500 | 0.042 |
| 41 64 30 1 26 294 376 388 415 490 500 | 0.039 | 41 90 126 135 201 236 294 376 388 500 | 0.042 |
| 41 64 90 1 26 201 294 376 388 415 500 | 0.039 | 29135142 201 242 454 742 766 772 775 | 0.042 |
| 41 90 126 135 236 294 376 388 415 500 | 0.039 | 34 64 201 242 428 680 742 766 772 787 | 0.042 |
| 34 41 90 135 201 236 294 376 388 500 | 0.039 | 34 36 41 135 311 667 680 742772 775 | 0.042 |
| 34 41 64 90 126 201 294 376 415 500 | 0.039 | 123 135 396 415 435 454 500 630 742 766 | 0.042 |
| 41 64 SO 201 236 294 376 388 415 500 | 0.039 | 29 138 242 428 454 742 766 772 776 789 | 0.042 |
| 34 41 90126 201 294 376 388 500 772 | 0.040 | 288 402 490 547 634 645 667 680 742 787 | 0.042 |
| 34 41 64 90 201 236 294 376 388 500 | 0.040 | 29 34 135 428 667 680 742 766 772 775 | 0.042 |
| 41 57 90 126 201 236 294 376 388 500 | 0.040 | 34 41 00 126 294 376 388 503 742 772 | 0.042 |
| 34 41 90 126 201 294 376 388 490 500 | 0.049 | 135 201 402 428 435 454 742 766 775 789 | 0.043 |
| 123 402 415 456490 645 867 880 742 788 | 0.040 | 34 41 64 90 126 234 376 388 490 772 | 0.043 |
| 34 41 242 415 667 680 742 788 772 789 | 0.040 | 34 41 90 1 26 294 378 388 490 500 772 | 0.043 |
| 29 34 135 435 667 680 742 766 772 787 | 0.040 | 29 135 396 402 428 435 454 490 766 775 | 0.043 |
| 34 41 64 90 201 234 378 415 500 775 | 0.040 | 29126 135 388 415 428 435 454 490 789 | 0.043 |
| 34 41 80 126 135 201 294 376 388 500 | 0.040 | 135 201 402 428 454 630 742 766 772 787 | 0.043 |
| 34 41 80 1 42 311 415 742 766 772 775 | 0.040 | 29 242 428 454 645 867 680 766 775 789 | 0.043 |
| 41 57 64 90 126 201 294 376 396 500 | 0.040 | 29 201 428 454 667 680 742 756 772 789 | 0.043 |
| 29 34 123 136 428 680 742 765 772 787 | 0.040 | 34 41 90 126 135 201 294 376 490 500 | 0.043 |
| 126 396415428454667 880 742 766 789 | 0.040 | 34 36 41 242 288 311 742 766 772 775 | 0.043 |
| 34 41 64 90 126 201 294 378 388 500 | 0.040 | 29 201428 435 454 667 680 742 786 789 | 0.043 |
| 34 41 64 90 126 201 294 388 SOO 772 | 0.041 | 57 64 90 1 26 201 294 376 388 490 500 | 0.043 |
| 57 90 126 201 294 376 388 415 456 500 | 0.041 | 8 19 296 338 415 466 680 766 775 789 | 0.043 |
| 34 36 126 428 645 680 742 768 772 775 | 0.041 | 29 135 201428 435 454 742 758 775 789 | 0.043 |
| 34 41 64 90 126 201 294 388 490 500 | 0.041 | 34 41 64 90 126 135 201 294 376 506 | 0.043 |
| 29 135 415 428 454 680 742 786 772 789 | 0.041 | 29135142 454 667 680 742 766 772 775 | 0.043 |
| 34 41 57 90 126 294 388 500 742 772 | 0.041 | 34 36 41 80 135 242 766 772 775 787 | 0.043 |
| 123 135142 242 296 402 456 657 680 787 | 0.041 | 29 123 236 428 454 742 766 772 775 789 | 0.043 |

| | | | |
|---|---|---|---|
| Feature ID | MAE | Feature © | MAE |
| 34 41 64 90126 294 376 338 500 772 | 0-043 | 34 135 402 630 634 645660 742 766 772 | 0.046 |
| 142 288 396 402 435 454 634 645666 775 | 0.043 | 135 201 428 435 454 674 860 742 766 789 | 0.046 |
| 34 41 64 90 1 26 294 388 500 742 772 | 0.044 | 8123 288 402 490 667 680 742 766 775 | 0.046 |
| 19126 135 386 396 428 454 667 680 769 | 0.044 | 123 201 3S6 402 428 435 454 766 775 789 | 0.046 |
| 135 242 396 402 415 634 645 667 674 680 | 0.044 | 205 258 338 402 456 490 736 788 787 789 | 0.046 |
| 126 1 35 423 454 645667 680 766 772 789 | 0.044 | 29 396 402 428 435 456 634 667 680 787 | 0.046 |
| 41 84 90 126 135 201294 376 388 500 | 0.044 | 123 435454 630 634 645 667 680 787 789 | 0.046 |
| 34 41 90 126 201 294 376 456 500 772 | 0.044 | 19 288 331 396 402 415 435 630 634 742 | 0.046 |
| 34 41 90 126 135 294 388 490 500 772 | 0.044 | 396 428 435 454 667 680 742 766 775 789 | 0.046 |
| 201 415 454 634 645 667 680 742 766 772 | 0.044 | 36 201 428 435 454 490 500 680 766 789 | 0.046 |
| 29 34 84 428 667 680 742 768 772 789 | 0.044 | 64 123 135 142 205 278 634 645 674 787 | 0.047 |
| 135142 428 454 630 742 766 772 775 789 | 0.044 | 29135142 242 388 428 454 490 707 789 | 0.047 |
| 29135 454 490 667 680 742 766 772 775 | 0.044 | 237 288 296 331 338 435 547 634 775 787 | 0.047 |
| 29123135 376 396 454 680 742 766 789 | 0.044 | 34 41 135 634 645 667 680 742 766 772 | 0.047 |
| 135 242 378 396 402 415 428 435 454 490 | 0.044 | 123 142 242 288 415 634 645 667 680 789 | 0.047 |
| 34 41 90 201 294 376 500 645 742 772 | 0.044 | 135 396 402 634 645 667 674 680 787 789 | 0.047 |
| 64 90 126 135 201 236 294 376 388 500 | 0.044 | 8 19 64 142 288 296 435 667 674 787 | 0.047 |
| 37 278 288 331 338 376 547 634 645 787 | 0.044 | 118 225 236 258 278 286 296 311 456 589 | 0.047 |
| 34 41 57 90 201 294 376 500 742 772 | 0.044 | 19 64 415 490 634 645 674 775 776 789 | 0.047 |
| 10 29 36 135 142 388 396435 454 789 | 0.044 | 8 64 123135142 278 415 634 645 674 | 0.047 |
| 90126135 201 238 294 376 388 490 500 | 0.044 | 29123 201 428 435 454 490 500 766 775 | 0.047 |
| 29123 135 142 396 415 435 454 766 775 | 0.944 | 19 36 201 428 435 454 742 766 775 788 | 0 047 |
| 29 306 428 435 454 490 680 742 766 789 | 0.045 | 29123 454 645667 680 742 766 772 789 | 0.047 |
| 29 64 135142 428 454 490 766 772 789 | 0.045 | 36 135 142 201 415 454 500 766 775 789 | 0.047 |
| 29 428 435 454 634 645 680 742 766 772 | 0.045 | 8 19 142 278 396 415 674 766 775 789 | 0.047 |
| 135 396 402 435 454 480 742 766 775 789 | 0.045 | 123 288 435 634 645 680 742 766 772 775 | 0.048 |
| 29135 454 634 845 667 680 742 766 772 | 0.045 | 34 41 311 630 634 645 667 680 742 772 | 0.048 |
| 29135 428 454 645 667 680 742 766 772 | 0.045 | 8 64 278 286 296 338 544 634 645 787 | 0.048 |
| 34 41 64 90 126 294 376 388 490 500 | 0.045 | 8 64 205 278 415 544 634 645 674 789 | 0.048 |
| 29 142 288 428 454 667 680 742 766 772 | 0.045 | 123 135 242 547 634 645 667 680 742 776 | 0.048 |
| 123 205 288 402 435 634 645 667 680 787 | 0.045 | 8 37 142 278 288 296 331 338 415 634 | 0.048 |
| 34 36 135 428 490 687 680 742 766 772 | 0.045 | 34 3641 80135242 311435 490 787 | 0.048 |
| 29 428 454 634 645 667 680 742 766 772 | 0.045 | 29 34 36 57135 242 428 772 775 789 | 0.048 |
| 29 38 8 428 454 634 645 667 680 742 772 | 0.045 | 34 135 428 834 845 667 680 742 7S6 772 | 0.048 |
| 29135 396 428 454 490 742 766 775 789 | 0.045 | 142 278 288 396 454 630 634 645 666 789 | 0.048 |
| 41 64 90 1 35 201 294 376 388 456 500 | 0.045 | 288 296 402 547 630 634 645 667 674 680 | 0.046 |
| 29142 396 428 435 454 490 766 775 789 | 0.045 | 8 123 135 142 242 396 402 456 490 787 | 0.048 |
| 29 135 415 428 435 454 500 766 775 789 | 0.045 | B 142 237 258 296 402 456 695 776 787 | 0.048 |
| 29135 428 454 490 680 742 766 772 789 | 0.045 | 123 242 288 428 634 645 667 680 766 775 | 0.048 |
| 19 135 142 415 428 454 490 766 775 789 | 0.045 | 142 278 288 396 428 454 634 645 775 789 | 0.049 |
| 64 205 225 278 296 416 544 634 645 674 | 0.045 | 83 205 225 236 286 402 456 544 589 708 | 0.049 |
| 29 135 142 428 454 667 680 742 768 772 | 0.045 | 123 242 288 396 402 034 045 607 680 742 | 0.049 |
| 29 34 288 428 435 667 680 742 766 772 | 0.045 | 142 205 278 288 396 415 435 634 645 742 | 0.049 |
| 123 278 288 396 415 435 454 456 766 789 | 0.045 | 8 288 296 415 456 634 645 667 680 766 | 0.049 |
| 29 388 428 435 454 645 667 680 742 772 | 0.045 | 36 225 288 428 500 667 674 680 787 789 | 0.049 |
| 8 142 415 435 645 667 674 680 766 775 | 0.045 | 135 376 402 415 428 435 454 5® 775 789 | 0.049 |
| 29135 428 454 490 634 645 680 766 772 | 0.045 | 29 123 135 396 454 667 674 680 787 789 | 0.049 |
| 64 90 126 201 236 294 376 388 490 500 | 0.045 | 8 123 242 288 296 415 742 766 775 789 | 0.049 |
| 64 90 125135 201 294 376 388 490 500 | 0.046 | 135 142 428 454 634 645 667 680 742 766 | 0.049 |
| 34 428 435 667 680 742 766 772 775 789 | 0.046 | 19 118 278 288 296 338 547 634 653 787 | 0.049 |
| 36135 428 435 454 645 667 680 742 766 | 0.046 | 19 64 123 142 288 396 415 674 742 769 | 0.049 |

| | |
|---|---|
| Feature ID | MAE |
| 8 29118 135 142 205 242 415 674 680 | 0.049 |
| 64 135142 242 396 415 456 634 645 667 | 0.049 |
| 142 288 396 435 454 430 630 634 645 666 | 0.049 |
| 142 242 338 416 544 634 645 667 680 787 | 0.049 |
| 9 211 236 258 296 348 376 544 621 666 | 0.049 |
| 258 357 402 415 456 544 621 666 674 695 | 0.049 |
| 142 278 331 396 428435 454 634 645 787 | 0.049 |
| 29 123142 205 288 634 645 667 680 787 | 0.049 |
| 8 19 64 205 278 288 296 338 653 787 | 0.049 |
| 29 36 64 135 142 428 435454 775 787 | 0.049 |
| 123 142 237 236 396 415 645 667 680 776 | 0.049 |
| 286 288 296 490 547 634 645 666 667 787 | 0.049 |
| 19 29 123 135 376 396 634 645 680 766 | 0.050 |
| 123 142 205 396 435 454 456 680 742 766 | 0.050 |
| 236 242 258 306 402 544 621 666 674 695 | 0.050 |
| 29 225 288 296 461 634 645 667 674 680 | 0.050 |
| 19 135142 396 435 454 674 775 787 789 | 0.050 |
| 8 80123 205 237 288 396 402 766 775 | 0.050 |
| 118 205 547 634 645 667 674 680 695 787 | 0.050 |
| 8 52 64 278 288 296 338 415 435 490 | 0.050 |
| 15 225 306 311 402 469 589 607 630 695 | 0.050 |

TABLE S21
Information on predictive pan-cancer features using in silico
samples generated with random subsets of healthy samples.

| ID | Transcript | Gene | Chr | Site | Region | Group | FPKM_{blood} |
|---|---|---|---|---|---|---|---|
| 8 | ENST00000221954.6 | CEACAM4 | 19 | 42133442 | promoter | blood | 32.81 |
| 9 | ENST00000225275.3 | MPO | 17 | 56358296 | promoter | blood | 7.23 |
| 15 | ENST00000262407.5 | ITGA2B | 17 | 42466873 | promoter | blood | 12.34 |
| 19 | ENST00000264260.6 | IL18RAP | 2 | 103035149 | promoter | blood | 42.11 |
| 29 | ENST00000299663.7 | CLEC4E | 12 | 8693559 | promoter | blood | 29.27 |
| 34 | ENST00000307395.4 | GP9 | 3 | 128779610 | promoter | blood | 9.72 |
| 36 | ENST00000310544.8 | PHOSPHO1 | 17 | 47307890 | promoter | blood | 30.92 |
| 37 | ENST00000312156.8 | NFE2 | 12 | 54689544 | promoter | blood | 11.15 |
| 41 | ENST00000324134.10 | NLRP12 | 19 | 54327597 | promoter | blood | 9.17 |
| 52 | ENST00000342063.4 | C19orf35 | 19 | 2282175 | promoter | blood | 8.64 |
| 57 | ENST00000355524.7 | FCAR | 19 | 55385736 | promoter | blood | 53.04 |
| 64 | ENST00000375448.4 | PADI4 | 1 | 17634692 | promoter | blood | 95.04 |
| 80 | ENST00000391726.7 | FCAR | 19 | 55385704 | promoter | blood | 6 |
| 83 | ENST00000392841.1 | HMBS | 11 | 118958697 | promoter | blood | 7.28 |
| 90 | ENST00000398632.3 | MX2 | 21 | 42774561 | promoter | blood | 7.28 |
| 118 | ENST00000452208.1 | RGL4 | 22 | 24038671 | promoter | blood | 64.94 |
| 123 | ENST00000460208.1 | LILRB3 | 19 | 54721567 | promoter | blood | 51.15 |
| 126 | ENST00000462275.5 | TBXAS1 | 7 | 139529072 | promoter | blood | 6.07 |
| 135 | ENST00000465984.5 | SLC11A1 | 2 | 219246911 | promoter | blood | 10.54 |
| 142 | ENST00000467972.5 | SLC12A9 | 7 | 100454416 | promoter | blood | 6.8 |
| 201 | ENST00000487761.5 | GPSM3 | 6 | 32160645 | promoter | blood | 14.37 |
| 205 | ENST00000488970.1 | BTK | X | 100609209 | promoter | blood | 7.4 |
| 211 | ENST00000490872.1 | SLC11A1 | 2 | 219254688 | promoter | blood | 26.15 |
| 225 | ENST00000496915.1 | PREX1 | 20 | 47259276 | promoter | blood | 5.23 |
| 236 | ENST00000509314.5 | FBXL5 | 4 | 15661487 | promoter | blood | 10.24 |
| 237 | ENST00000509339.1 | MXD3 | 5 | 176735063 | promoter | blood | 30.07 |
| 242 | ENST00000517813.1 | DENND3 | 8 | 142194587 | promoter | blood | 13.47 |
| 258 | ENST00000523369.1 | LCP2 | 5 | 169685502 | promoter | blood | 9.88 |
| 278 | ENST00000533815.2 | BTNL8 | 5 | 180336564 | promoter | blood | 5.72 |
| 286 | ENST00000538842.1 | ATG16L2 | 11 | 72527790 | promoter | blood | 6.35 |
| 288 | ENST00000539932.5 | SLC11A1 | 2 | 219246926 | promoter | blood | 50.32 |
| 294 | ENST00000542481.1 | ATG16L2 | 11 | 72534940 | promoter | blood | 25.18 |
| 296 | ENST00000543576.5 | DENND1C | 19 | 6481798 | promoter | blood | 12.06 |
| 306 | ENST00000554736.5 | GNG2 | 14 | 52328042 | promoter | blood | 9.3 |
| 311 | ENST00000559341.5 | MAN2A2 | 15 | 91459225 | promoter | blood | 8.61 |
| 331 | ENST00000566082.1 | USB1 | 16 | 58049017 | promoter | blood | 6.82 |
| 338 | ENST00000570439.1 | ACAP1 | 17 | 7248650 | promoter | blood | 5.27 |
| 348 | ENST00000574548.1 | RNF167 | 17 | 4847675 | promoter | blood | 6.4 |
| 357 | ENST00000581974.1 | DHRS13 | 17 | 27226541 | promoter | blood | 5.97 |
| 376 | ENST00000590974.1 | LYL1 | 19 | 13213975 | promoter | blood | 5.48 |
| 388 | ENST00000597611.7 | FKBP8 | 19 | 18654887 | promoter | blood | 9.25 |
| 396 | ENST00000599180.2 | FFAR2 | 19 | 35939203 | promoter | blood | 24.79 |
| 402 | ENST00000601502.1 | MYO1F | 19 | 8610602 | promoter | blood | 8.05 |
| 415 | ENST00000615340.4 | RASGRP4 | 19 | 38916837 | promoter | blood | 6.88 |
| 416 | ENST00000616356.4 | FCN1 | 9 | 137809723 | promoter | blood | 16.99 |
| 428 | ENST00000221954.6 | CEACAM4 | 19 | 42133268 | junction | blood | 32.81 |
| 435 | ENST00000262407.5 | ITGA2B | 17 | 42466654 | junction | blood | 12.34 |
| 454 | ENST00000307395.4 | GP9 | 3 | 128779693 | junction | blood | 9.72 |
| 456 | ENST00000310544.8 | PHOSPHO1 | 17 | 47307830 | junction | blood | 30.92 |
| 461 | ENST00000324134.10 | NLRP12 | 19 | 54327140 | junction | blood | 9.17 |
| 469 | ENST00000338372.6 | VSTM1 | 19 | 54566998 | junction | blood | 10.71 |
| 490 | ENST00000380299.3 | HBD | 11 | 5255572 | junction | blood | 138.84 |
| 500 | ENST00000394430.5 | RASGRP2 | 11 | 64510266 | junction | blood | 17.61 |
| 544 | ENST00000465984.5 | SLC11A1 | 2 | 219247098 | junction | blood | 10.54 |
| 547 | ENST00000466612.5 | ABTB1 | 3 | 127392451 | junction | blood | 6.18 |
| 589 | ENST00000484762.1 | CSF3R | 1 | 36933423 | junction | blood | 8.26 |
| 607 | ENST00000488970.1 | BTK | X | 100608858 | junction | blood | 7.4 |
| 621 | ENST00000496915.1 | PREX1 | 20 | 47258945 | junction | blood | 5.23 |
| 630 | ENST00000509314.5 | FBXL5 | 4 | 15661350 | junction | blood | 10.24 |
| 634 | ENST00000513001.5 | ACSL1 | 4 | 185678973 | junction | blood | 17.65 |
| 645 | ENST00000521442.1 | ATP6V1B2 | 8 | 20075793 | junction | blood | 8.33 |
| 653 | ENST00000526387.5 | TBC1D10C | 11 | 67171411 | junction | blood | 7.1 |
| 666 | ENST00000532897.5 | MKNK1 | 1 | 47037735 | junction | blood | 6.23 |
| 667 | ENST00000533815.2 | BTNL8 | 5 | 180336697 | junction | blood | 5.72 |
| 674 | ENST00000538842.1 | ATG16L2 | 11 | 72527872 | junction | blood | 6.35 |
| 680 | ENST00000542481.1 | ATG16L2 | 11 | 72535167 | junction | blood | 25.18 |
| 695 | ENST00000559341.5 | MAN2A2 | 15 | 91459486 | junction | blood | 8.61 |
| 708 | ENST00000564894.1 | RNF166 | 16 | 88767670 | junction | blood | 7.9 |
| 736 | ENST00000581974.1 | DHRS13 | 17 | 27226145 | junction | blood | 5.97 |
| 742 | ENST00000585852.5 | FMNL1 | 17 | 43308054 | junction | blood | 19.81 |
| 766 | ENST00000597611.7 | FKBP8 | 19 | 18654719 | junction | blood | 9.25 |
| 772 | ENST00000599180.2 | FFAR2 | 19 | 35939281 | junction | blood | 24.79 |
| 775 | ENST00000599716.5 | SHKBP1 | 19 | 41082891 | junction | blood | 10.66 |
| 776 | ENST00000600463.1 | IFI30 | 19 | 18286032 | junction | blood | 6.41 |
| 787 | ENST00000614135.4 | RASGRP4 | 19 | 38916709 | junction | blood | 8.18 |
| 789 | ENST00000616356.4 | FCN1 | 9 | 137809615 | junction | blood | 16.99 |

Columns
ID: feature index
Transcript: transcript ID
Gene: gene name
Chr: chromosome
Site: coordinate of nucleosome-depleted site (GRCh37)
Region: location of nucleosome-depleted site
Group: gene group based on its expression in blood and tumor
FPKM_{blood}: FPKM value in normal blood. Their FPKM values in tumors of 20 cancer types are all <1.

### Supplementary Data 1

| | | | | | |
|---|---|---|---|---|---|
| chr1 | 11106604 | 11107500 | 466_105938_2475(MTOR)_59a | 0 | — |
| chr1 | 11108180 | 11108286 | 466_105937_2475(MTOR)_58a | 0 | — |
| chr1 | 11109289 | 11109370 | 466_105936_2475(MTOR)_57a | 0 | — |
| chr1 | 11109648 | 11109729 | 466_105935_2475(MTOR)_56a | 0 | — |
| chr1 | 11112851 | 11112917 | 466_105934_2475(MTOR)_55a | 0 | — |
| chr1 | 11114317 | 11114453 | 466_105933_2475(MTOR)_54a | 0 | — |
| chr1 | 11114812 | 11114887 | 466_105932_2475(MTOR)_53a | 0 | — |
| chr1 | 11115395 | 11115468 | 466_105931_2475(MTOR)_52a | 0 | — |
| chr1 | 11117003 | 11117086 | 466_105930_2475(MTOR)_51a | 0 | — |
| chr1 | 11121245 | 11121368 | 466_105929_2475(MTOR)_50a | 0 | — |
| chr1 | 11121978 | 11122126 | 466_105928_2475(MTOR)_49a | 0 | — |
| chr1 | 11124497 | 11124633 | 466_105927_2475(MTOR)_48a | 0 | — |
| chr1 | 11126621 | 11126796 | 466_105926_2475(MTOR)_47a | 0 | — |
| chr1 | 11127009 | 11127144 | 466_105925_2475(MTOR)_46a | 0 | — |
| chr1 | 11127623 | 11127806 | 466_105924_2475(MTOR)_45a | 0 | — |
| chr1 | 11128003 | 11128126 | 466_105923_2475(MTOR)_44a | 0 | — |
| chr1 | 11128453 | 11128552 | 466_105922_2475(MTOR)_43a | 0 | — |
| chr1 | 11128854 | 11128951 | 466_105921_2475(MTOR)_42a | 0 | — |
| chr1 | 11129737 | 11129838 | 466_105920_2475(MTOR)_41a | 0 | — |
| chr1 | 11130528 | 11130777 | 466_105919_2475(MTOR)_40a | 0 | — |
| chr1 | 11133079 | 11133197 | 466_105918_2475(MTOR)_39a | 0 | — |
| chr1 | 11134350 | 11134466 | 466_105917_2475(MTOR)_38a | 0 | — |
| chr1 | 11139303 | 11139435 | 466_105916_2475(MTOR)_37a | 0 | — |
| chr1 | 11139528 | 11139658 | 466_105915_2475(MTOR)_36a | 0 | — |
| chr1 | 11144647 | 11144755 | 466_105914_2475(MTOR)_35a | 0 | — |
| chr1 | 11144967 | 11145045 | 466_105913_2475(MTOR)_34a | 0 | — |
| chr1 | 11146675 | 11146791 | 466_105912_2475(MTOR)_33a | 0 | — |
| chr1 | 11150125 | 11150226 | 466_105911_2475(MTOR)_32a | 0 | — |
| chr1 | 11157151 | 11157291 | 466_105910_2475(MTOR)_31a | 0 | — |
| chr1 | 11167441 | 11167517 | 466_105909_2475(MTOR)_30a | 0 | — |
| chr1 | 11199257 | 11199403 | 466_105908_2475(MTOR)_29a | 0 | — |
| chr1 | 11199540 | 11199703 | 466_105907_2475(MTOR)_28a | 0 | — |
| chr1 | 11204560 | 11204703 | 466_105906_2475(MTOR)_27a | 0 | — |
| chr1 | 11209311 | 11209458 | 466_105905_2475(MTOR)_26a | 0 | — |
| chr1 | 11210813 | 11210906 | 466_105904_2475(MTOR)_25a | 0 | — |
| chr1 | 11212311 | 11212474 | 466_105903_2475(MTOR)_24a | 0 | — |
| chr1 | 11212795 | 11212908 | 466_105902_2475(MTOR)_23a | 0 | — |
| chr1 | 11213398 | 11213566 | 466_105901_2475(MTOR)_22a | 0 | — |
| chr1 | 11216147 | 11216234 | 466_105900_2475(MTOR)_21a | 0 | — |
| chr1 | 11228667 | 11228918 | 466_105899_2475(MTOR)_20a | 0 | — |
| chr1 | 11230924 | 11231054 | 466_105898_2475(MTOR)_19a | 0 | — |
| chr1 | 11231299 | 11231434 | 466_105897_2475(MTOR)_18a | 0 | — |
| chr1 | 11232435 | 11232528 | 466_105896_2475(MTOR)_17a | 0 | — |
| chr1 | 11233397 | 11233487 | 466_105895_2475(MTOR)_16a | 0 | — |
| chr1 | 11234142 | 11234265 | 466_105894_2475(MTOR)_15a | 0 | — |
| chr1 | 11237842 | 11238048 | 466_105893_2475(MTOR)_14a | 0 | — |
| chr1 | 11238401 | 11238617 | 466_105892_2475(MTOR)_13a | 0 | — |
| chr1 | 11240302 | 11240547 | 466_105891_2475(MTOR)_12a | 0 | — |
| chr1 | 11241552 | 11241681 | 466_105890_2475(MTOR)_11a | 0 | — |
| chr1 | 11243113 | 11243300 | 466_105889_2475(MTOR)_10a | 0 | — |
| chr1 | 11247624 | 11247733 | 466_105888_2475(MTOR)_9a | 0 | — |
| chr1 | 11247818 | 11248094 | 466_105887_2475(MTOR)_8a | 0 | — |
| chr1 | 11253838 | 11253973 | 466_105886_2475(MTOR)_7a | 0 | — |
| chr1 | 11255991 | 11256192 | 466_105885_2475(MTOR)_6a | 0 | — |
| chr1 | 11256284 | 11256302 | 466_105884_2475(MTOR)_5a | 0 | — |
| chr1 | 11256932 | 11257165 | 466_105883_2475(MTOR)_4a | 0 | — |
| chr1 | 11258484 | 11258593 | 466_105882_2475(MTOR)_3a | 0 | — |
| chr1 | 11259247 | 11259415 | 466_105881_2475(MTOR)_2a | 0 | — |
| chr1 | 26696403 | 26697540 | 426_4218_8289(ARID1A)_1a | 0 | — |
| chr1 | 26729650 | 26729863 | 426_4219_8289(ARID1A)_2a | 0 | — |
| chr1 | 26731151 | 26731604 | 426_4220_8289(ARID1A)_3a | 0 | — |
| chr1 | 26732675 | 26732792 | 426_4221_8289(ARID1A)_4a | 0 | — |
| chr1 | 26760855 | 26761096 | 426_4222_8289(ARID1A)_5a | 0 | — |
| chr1 | 26761383 | 26761473 | 426_4223_8289(ARID1A)_6a | 0 | — |
| chr1 | 26762151 | 26762319 | 426_4224_8289(ARID1A)_7a | 0 | — |
| chr1 | 26762972 | 26763285 | 426_4225_8289(ARID1A)_8a | 0 | — |
| chr1 | 26766220 | 26766366 | 426_4226_8289(ARID1A)_9a | 0 | — |
| chr1 | 26766456 | 26766566 | 426_4227_8289(ARID1A)_10a | 0 | — |
| chr1 | 26767789 | 26767999 | 426_4228_8289(ARID1A)_11a | 0 | — |
| chr1 | 26771118 | 26771326 | 426_4229_8289(ARID1A)_12a | 0 | — |
| chr1 | 26772499 | 26772632 | 426_4230_8289(ARID1A)_13a | 0 | — |
| chr1 | 26772811 | 26772987 | 426_4231_8289(ARID1A)_14a | 0 | — |
| chr1 | 26773345 | 26773496 | 426_4232_8289(ARID1A)_15a | 0 | — |
| chr1 | 26773579 | 26773717 | 426_4233_8289(ARID1A)_16a | 0 | — |
| chr1 | 26773801 | 26773898 | 426_4234_8289(ARID1A)_17a | 0 | — |
| chr1 | 26774328 | 26775220 | 426_4235_8289(ARID1A)_18a | 0 | — |
| chr1 | 26775576 | 26775707 | 426_4236_8289(ARID1A)_19a | 0 | — |
| chr1 | 26779022 | 26780756 | 426_4237_8289(ARID1A)_20a | 0 | — |
| chr1 | 43337848 | 43337927 | 466_106798_4352(MPL)_1a | 0 | — |
| chr1 | 43338098 | 43338231 | 466_106799_4352(MPL)_2a | 0 | — |
| chr1 | 43338541 | 43338720 | 466_106800_4352(MPL)_3a | 0 | — |
| chr1 | 43339270 | 43339569 | 466_106801_4352(MPL)_4a | 0 | — |
| chr1 | 43339963 | 43340126 | 466_106802_4352(MPL)_5a | 0 | — |
| chr1 | 43340386 | 43340513 | 466_106803_4352(MPL)_6a | 0 | — |
| chr1 | 43346444 | 43346629 | 466_106804_4352(MPL)_7a | 0 | — |
| chr1 | 43346791 | 43346934 | 466_106805_4352(MPL)_8a | 0 | — |
| chr1 | 43348842 | 43349002 | 466_106806_4352(MPL)_9a | 0 | — |
| chr1 | 43349262 | 43349359 | 466_106807_4352(MPL)_10a | 0 | — |
| chr1 | 43352215 | 43352303 | 466_106808_4352(MPL)_11a | 0 | — |
| chr1 | 43352517 | 43352772 | 466_106809_4352(MPL)_12a | 0 | — |
| chr1 | 64834561 | 64834657 | 463_81647_3716(JAK1)_26a | 0 | — |
| chr1 | 64835395 | 64835506 | 463_81646_3716(JAK1)_25a | 0 | — |
| chr1 | 64836097 | 64836215 | 463_81645_3716(JAK1)_24a | 0 | — |
| chr1 | 64837931 | 64838104 | 463_81644_3716(JAK1)_23a | 0 | — |
| chr1 | 64838464 | 64838589 | 463_81643_3716(JAK1)_22a | 0 | — |
| chr1 | 64839602 | 64839795 | 463_81642_3716(JAK1)_21a | 0 | — |
| chr1 | 64841244 | 64841339 | 463_81641_3716(JAK1)_20a | 0 | — |
| chr1 | 64841450 | 64841601 | 463_81640_3716(JAK1)_19a | 0 | — |
| chr1 | 64844063 | 64844215 | 463_81639_3716(JAK1)_18a | 0 | — |
| chr1 | 64844753 | 64844889 | 463_81638_3716(JAK1)_17a | 0 | — |
| chr1 | 64845512 | 64845640 | 463_81637_3716(JAK1)_16a | 0 | — |
| chr1 | 64846648 | 64846736 | 463_81636_3716(JAK1)_15a | 0 | — |
| chr1 | 64847531 | 64847675 | 463_81635_3716(JAK1)_14a | 0 | — |
| chr1 | 64850803 | 64850910 | 463_81634_3716(JAK1)_13a | 0 | — |
| chr1 | 64855508 | 64855698 | 463_81633_3716(JAK1)_12a | 0 | — |
| chr1 | 64857655 | 64857779 | 463_81632_3716(JAK1)_11a | 0 | — |
| chr1 | 64860104 | 64860262 | 463_81631_3716(JAK1)_10a | 0 | — |
| chr1 | 64864786 | 64864972 | 463_81630_3716(JAK1)_9a | 0 | — |
| chr1 | 64866865 | 64867208 | 463_81629_3716(JAK1)_8a | 0 | — |
| chr1 | 64869310 | 64869474 | 463_81628_3716(JAK1)_7a | 0 | — |
| chr1 | 64873369 | 64873523 | 463_81627_3716(JAK1)_6a | 0 | — |
| chr1 | 64879024 | 64879148 | 463_81626_3716(JAK1)_5a | 0 | — |
| chr1 | 64883276 | 64883475 | 463_81625_3716(JAK1)_4a | 0 | — |
| chr1 | 64886258 | 64886341 | 463_81624_3716(JAK1)_3a | 0 | — |
| chr1 | 113391033 | 113391349 | 465_103736_260425(MAGI3)_1 | 0 | — |
| chr1 | 113549514 | 113549631 | 465_103737_260425(MAGI3)_2 | 0 | — |
| chr1 | 113580541 | 113580661 | 465_103738_260425(MAGI3)_3 | 0 | — |
| chr1 | 113585386 | 113585596 | 465_103739_260425(MAGI3)_4 | 0 | — |
| chr1 | 113590483 | 113590658 | 465_103740_260425(MAGI3)_5 | 0 | — |
| chr1 | 113594480 | 113594560 | 465_103741_260425(MAGI3)_6 | 0 | — |
| chr1 | 113614600 | 113614658 | 465_103742_260425(MAGI3)_7 | 0 | — |
| chr1 | 113618531 | 113618606 | 465_103743_260425(MAGI3)_8 | 0 | — |
| chr1 | 113619735 | 113619830 | 465_103744_260425(MAGI3)_9 | 0 | — |
| chr1 | 113622805 | 113622994 | 465_103745_260425(MAGI3)_1 | 0 | — |
| chr1 | 113641910 | 113642516 | 465_103746_260425(MAGI3)_11 | 0 | — |
| chr1 | 113643742 | 113643774 | 465_103747_260425(MAGI3)_12 | 0 | — |
| chr1 | 113646485 | 113646642 | 465_103748_260425(MAGI3)_13 | 0 | — |
| chr1 | 113649236 | 113649328 | 465_103749_260425(MAGI3)_14 | 0 | — |
| chr1 | 113651013 | 113651206 | 465_103750_260425(MAGI3)_15 | 0 | — |
| chr1 | 113653829 | 113654018 | 465_103751_260425(MAGI3)_16 | 0 | — |
| chr1 | 113659079 | 113659265 | 465_103752_260425(MAGI3)_17 | 0 | — |
| chr1 | 113671733 | 113671836 | 465_103753_260425(MAGI3)_18 | 0 | — |
| chr1 | 113672614 | 113672741 | 465_103754_260425(MAGI3)_19 | 0 | — |
| chr1 | 113673321 | 113673465 | 465_103755_260425(MAGI3)_20 | 0 | — |
| chr1 | 113681197 | 113681336 | 465_103756_260425(MAGI3)_21 | 0 | — |
| chr1 | 113682211 | 113684014 | 465_103757_260425(MAGI3)_22 | 0 | — |
| chr1 | 114708534 | 114708654 | 469_119576_4893(NRAS)_5 | 0 | — |
| chr1 | 114709568 | 114709728 | 469_119575_4893(NRAS)_4 | 0 | — |
| chr1 | 114713799 | 114713978 | 469_119574_4893(NRAS)_3 | 0 | — |
| chr1 | 114716049 | 114716160 | 469_119573_4893(NRAS)_2 | 0 | — |
| chr1 | 162711291 | 162711300 | 431_48982_4921(DDR2)_4a | 0 | — |
| chr1 | 162719036 | 162719145 | 431_48983_4921(DDR2)_5a | 0 | — |
| chr1 | 162719190 | 162719214 | 431_48984_4921(DDR2)_6a | 0 | — |
| chr1 | 162741585 | 162741712 | 431_48985_4921(DDR2)_7a | 0 | — |
| chr1 | 162753094 | 162753197 | 431_48986_4921(DDR2)_8a | 0 | — |
| chr1 | 162754623 | 162754855 | 431_48987_4921(DDR2)_9a | 0 | — |
| chr1 | 162755155 | 162755303 | 431_48988_4921(DDR2)_10a | 0 | — |
| chr1 | 162755663 | 162755769 | 431_48989_4921(DDR2)_11a | 0 | — |
| chr1 | 162759795 | 162759979 | 431_48990_4921(DDR2)_12a | 0 | — |
| chr1 | 162761210 | 162761454 | 431_48991_4921(DDR2)_13a | 0 | — |
| chr1 | 162766000 | 162766063 | 431_48992_4921(DDR2)_14a | 0 | — |
| chr1 | 162767228 | 162767359 | 431_48993_4921(DDR2)_15a | 0 | — |
| chr1 | 162770301 | 162770512 | 431_48994_4921(DDR2)_16a | 0 | — |
| chr1 | 162772023 | 162772247 | 431_48995_4921(DDR2)_17a | 0 | — |
| chr1 | 162773468 | 162773596 | 431_48996_4921(DDR2)_18a | 0 | — |
| chr1 | 162775651 | 162775843 | 431_48997_4921(DDR2)_19a | 0 | — |
| chr1 | 162776135 | 162776370 | 431_48998_4921(DDR2)_20a | 0 | — |
| chr1 | 162778579 | 162778729 | 431_48999_4921(DDR2)_21a | 0 | — |
| chr1 | 162780111 | 162780246 | 431_49000_4921(DDR2)_22a | 0 | — |
| chr1 | 204525327 | 204525346 | 466_106137_4194(MDM4)_2a | 0 | — |
| chr1 | 204525483 | 204525596 | 466_106138_4194(MDM4)_3a | 0 | — |
| chr1 | 204526359 | 204526434 | 466_106139_4194(MDM4)_4a | 0 | — |
| chr1 | 204530683 | 204530817 | 466_106140_4194(MDM4)_5a | 0 | — |
| chr1 | 204532190 | 204532246 | 466_106141_4194(MDM4)_6a | 0 | — |
| chr1 | 204537429 | 204537497 | 466_106142_4194(MDM4)_7a | 0 | — |
| chr1 | 204538208 | 204538308 | 466_106143_4194(MDM4)_8a | 0 | — |
| chr1 | 204542783 | 204542944 | 466_106144_4194(MDM4)_9a | 0 | — |
| chr1 | 204544534 | 204544684 | 466_106145_4194(MDM4)_10a | 0 | — |
| chr1 | 204546796 | 204546877 | 466_106146_4194(MDM4)_11a | 0 | — |
| chr1 | 204549112 | 204549682 | 466_106147_4194(MDM4)_12a | 0 | — |
| chr10 | 8055655 | 8055896 | 462_69261_2625(GATA3)_4a | 0 | — |
| chr10 | 8058304 | 8058841 | 462_69262_2625(GATA3)_5a | 0 | — |
| chr10 | 8063992 | 8064138 | 462_69263_2625(GATA3)_6a | 0 | — |
| chr10 | 8069472 | 8069598 | 462_69264_2625(GATA3)_7a | 0 | — |
| chr10 | 8073738 | 8074023 | 462_69265_2625(GATA3)_8a | 0 | — |
| chr10 | 43077258 | 43077331 | 473_148919_5979(RET)_1a | 0 | — |
| chr10 | 43100458 | 43100722 | 473_148920_5979(RET)_2a | 0 | — |
| chr10 | 43102341 | 43102629 | 473_148921_5979(RET)_3a | 0 | — |
| chr10 | 43104951 | 43105193 | 473_148922_5979(RET)_4a | 0 | — |
| chr10 | 43106375 | 43106571 | 473_148923_5979(RET)_5a | 0 | — |
| chr10 | 43109030 | 43109230 | 473_148924_5979(RET)_6a | 0 | — |
| chr10 | 43111206 | 43111465 | 473_148925_5979(RET)_7a | 0 | — |
| chr10 | 43112098 | 43112224 | 473_148926_5979(RET)_8a | 0 | — |
| chr10 | 43112852 | 43112963 | 473_148927_5979(RET)_9a | 0 | — |
| chr10 | 43113555 | 43113675 | 473_148928_5979(RET)_10a | 0 | — |
| chr10 | 43114479 | 43114736 | 473_148929_5979(RET)_11a | 0 | — |
| chr10 | 43116583 | 43116731 | 473_148930_5979(RET)_12a | 0 | — |
| chr10 | 43118372 | 43118480 | 473_148931_5979(RET)_13a | 0 | — |
| chr10 | 43119530 | 43119745 | 473_148932_5979(RET)_14a | 0 | — |
| chr10 | 43120080 | 43120203 | 473_148933_5979(RET)_15a | 0 | — |
| chr10 | 43121945 | 43122016 | 473_148934_5979(RET)_16a | 0 | — |
| chr10 | 43123670 | 43123808 | 473_148935_5979(RET)_17a | 0 | — |
| chr10 | 43124882 | 43124982 | 473_148936_5979(RET)_18a | 0 | — |
| chr10 | 43126574 | 43127504 | 473_148937_5979(RET)_19a | 0 | — |
| chr10 | 43128111 | 43128269 | 473_148938_5979(RET)_20a | 0 | — |
| chr10 | 87864468 | 87864547 | 472_139786_5728(PTEN)_1 | 0 | — |
| chr10 | 87894024 | 87894109 | 472_139787_5728(PTEN)_2 | 0 | — |
| chr10 | 87925512 | 87925557 | 472_139788_5728(PTEN)_3 | 0 | — |
| chr10 | 87931045 | 87931089 | 472_139789_5728(PTEN)_4 | 0 | — |
| chr10 | 87933012 | 87933251 | 472_139790_5728(PTEN)_5 | 0 | — |
| chr10 | 87952117 | 87952259 | 472_139791_5728(PTEN)_6 | 0 | — |
| chr10 | 87957852 | 87958019 | 472_139792_5728(PTEN)_7 | 0 | — |
| chr10 | 87960893 | 87961118 | 472_139793_5728(PTEN)_8 | 0 | — |
| chr10 | 87965286 | 87965472 | 472_139794_5728(PTEN)_9 | 0 | — |
| chr10 | 121479580 | 121480021 | 461_60371_2263(FGFR2)_21a | 0 | — |
| chr10 | 121481850 | 121482177 | 461_60370_2263(FGFR2)_20a | 0 | — |
| chr10 | 121483697 | 121483803 | 461_60369_2263(FGFR2)_19a | 0 | — |
| chr10 | 121485394 | 121485532 | 461_60368_2263(FGFR2)_18a | 0 | — |
| chr10 | 121487353 | 121487424 | 461_60367_2263(FGFR2)_17a | 0 | — |
| chr10 | 121487990 | 121488113 | 461_60366_2263(FGFR2)_16a | 0 | — |
| chr10 | 121496531 | 121496722 | 461_60365_2263(FGFR2)_15a | 0 | — |
| chr10 | 121498494 | 121498605 | 461_60364_2263(FGFR2)_14a | 0 | — |
| chr10 | 121500825 | 121500947 | 461_60363_2263(FGFR2)_13a | 0 | — |
| chr10 | 121503789 | 121503941 | 461_60362_2263(FGFR2)_12a | 0 | — |
| chr10 | 121515116 | 121515319 | 461_60361_2263(FGFR2)_11a | 0 | — |
| chr10 | 121517318 | 121517463 | 461_60360_2263(FGFR2)_10a | 0 | — |
| chr10 | 121518681 | 121518829 | 461_60359_2263(FGFR2)_9a | 0 | — |
| chr10 | 121519978 | 121520169 | 461_60358_2263(FGFR2)_8a | 0 | — |
| chr10 | 121538591 | 121538715 | 461_60357_2263(FGFR2)_7a | 0 | — |
| chr10 | 121551289 | 121551459 | 461_60356_2263(FGFR2)_6a | 0 | — |
| chr10 | 121564501 | 121564579 | 461_60355_2263(FGFR2)_5a | 0 | — |
| chr10 | 121565437 | 121565704 | 461_60354_2263(FGFR2)_4a | 0 | — |
| chr10 | 121593708 | 121593817 | 461_60353_2263(FGFR2)_3a | 0 | — |
| chr11 | 532588 | 532755 | 463_79038_3265(HRAS)_6a | 0 | — |
| chr11 | 533276 | 533358 | 463_79037_3265(HRAS)_5a | 0 | — |
| chr11 | 533452 | 533612 | 463_79036_3265(HRAS)_4a | 0 | — |
| chr11 | 533765 | 533944 | 463_79035_3265(HRAS)_3a | 0 | — |
| chr11 | 534211 | 534322 | 463_79034_3265(HRAS)_2a | 0 | — |
| chr11 | 108227624 | 108227696 | 427_14211_472(ATM)_5 | 0 | — |
| chr11 | 108227775 | 108227888 | 427_14212_472(ATM)_6 | 0 | — |
| chr11 | 108229177 | 108229323 | 427_14213_472(ATM)_7 | 0 | — |
| chr11 | 108235669 | 108235834 | 427_14214_472(ATM)_8 | 0 | — |
| chr11 | 108243952 | 108244118 | 427_14215_472(ATM)_9 | 0 | — |
| chr11 | 108244787 | 108245026 | 427_14216_472(ATM)_10 | 0 | — |
| chr11 | 108246543 | 108246560 | 427_14217_472(ATM)_11 | 0 | — |
| chr11 | 108246963 | 108247127 | 427_14218_472(ATM)_12 | 0 | — |
| chr11 | 108248932 | 108249102 | 427_14219_472(ATM)_13 | 0 | — |
| chr11 | 108250700 | 108251072 | 427_14220_472(ATM)_14 | 0 | — |
| chr11 | 108251836 | 108252031 | 427_14221_472(ATM)_15 | 0 | — |
| chr11 | 108252816 | 108252912 | 427_14222_472(ATM)_16 | 0 | — |
| chr11 | 108253813 | 108254039 | 427_14223_472(ATM)_17 | 0 | — |
| chr11 | 108256214 | 108256340 | 427_14224_472(ATM)_18 | 0 | — |
| chr11 | 108257480 | 108257606 | 427_14225_472(ATM)_19 | 0 | — |
| chr11 | 108258985 | 108259075 | 427_14226_472(ATM)_20 | 0 | — |
| chr11 | 108267170 | 108267342 | 427_14227_472(ATM)_21 | 0 | — |
| chr11 | 108268409 | 108268609 | 427_14228_472(ATM)_22 | 0 | — |
| chr11 | 108271063 | 108271146 | 427_14229_472(ATM)_23 | 0 | — |
| chr11 | 108271250 | 108271406 | 427_14230_472(ATM)_24 | 0 | — |
| chr11 | 108272531 | 108272607 | 427_14231_472(ATM)_25 | 0 | — |
| chr11 | 108272721 | 108272852 | 427_14232_472(ATM)_26 | 0 | — |
| chr11 | 108279490 | 108279608 | 427_14233_472(ATM)_27 | 0 | — |
| chr11 | 108280994 | 108281168 | 427_14234_472(ATM)_28 | 0 | — |
| chr11 | 108282709 | 108282879 | 427_14235_472(ATM)_29 | 0 | — |
| chr11 | 108284226 | 108284473 | 427_14236_472(ATM)_30 | 0 | — |
| chr11 | 108287599 | 108287715 | 427_14237_472(ATM)_31 | 0 | — |
| chr11 | 108288976 | 108289103 | 427_14238_472(ATM)_32 | 0 | — |
| chr11 | 108289601 | 108289801 | 427_14239_472(ATM)_33 | 0 | — |
| chr11 | 108292618 | 108292793 | 427_14240_472(ATM)_34 | 0 | — |
| chr11 | 108293312 | 108293477 | 427_14241_472(ATM)_35 | 0 | — |
| chr11 | 108294926 | 108295059 | 427_14242_472(ATM)_36 | 0 | — |
| chr11 | 108297286 | 108297382 | 427_14243_472(ATM)_37 | 0 | — |
| chr11 | 108299713 | 108299885 | 427_14244_472(ATM)_38 | 0 | — |
| chr11 | 108301647 | 108301789 | 427_14245_472(ATM)_39 | 0 | — |
| chr11 | 108302852 | 108303029 | 427_14246_472(ATM)_40 | 0 | — |
| chr11 | 108304674 | 108304852 | 427_14247_472(ATM)_41 | 0 | — |
| chr11 | 108307896 | 108307984 | 427_14248_472(ATM)_42 | 0 | — |
| chr11 | 108310159 | 108310315 | 427_14249_472(ATM)_43 | 0 | — |
| chr11 | 108312410 | 108312498 | 427_14250_472(ATM)_44 | 0 | — |
| chr11 | 108315822 | 108315911 | 427_14251_472(ATM)_45 | 0 | — |
| chr11 | 108316010 | 108316113 | 427_14252_472(ATM)_46 | 0 | — |
| chr11 | 108317372 | 108317521 | 427_14253_472(ATM)_47 | 0 | — |
| chr11 | 108319953 | 108320058 | 427_14254_472(ATM)_48 | 0 | — |
| chr11 | 108321300 | 108321420 | 427_14255_472(ATM)_49 | 0 | — |
| chr11 | 108325309 | 108325544 | 427_14256_472(ATM)_50 | 0 | — |
| chr11 | 108326057 | 108326225 | 427_14257_472(ATM)_51 | 0 | — |
| chr11 | 108327644 | 108327758 | 427_14258_472(ATM)_52 | 0 | — |
| chr11 | 108329020 | 108329238 | 427_14259_472(ATM)_53 | 0 | — |
| chr11 | 108330213 | 108330421 | 427_14260_472(ATM)_54 | 0 | — |
| chr11 | 108331443 | 108331557 | 427_14261_472(ATM)_55 | 0 | — |
| chr11 | 108331878 | 108332037 | 427_14262_472(ATM)_56 | 0 | — |
| chr11 | 108332761 | 108332900 | 427_14263_472(ATM)_57 | 0 | — |
| chr11 | 108333885 | 108333968 | 427_14264_472(ATM)_58 | 0 | — |
| chr11 | 108334968 | 108335109 | 427_14265_472(ATM)_59 | 0 | — |
| chr11 | 108335844 | 108335961 | 427_14266_472(ATM)_60 | 0 | — |
| chr11 | 108343221 | 108343371 | 427_14267_472(ATM)_61 | 0 | — |
| chr11 | 108345742 | 108345908 | 427_14268_472(ATM)_62 | 0 | — |
| chr11 | 108347278 | 108347365 | 427_14269_472(ATM)_63 | 0 | — |
| chr11 | 108353765 | 108353880 | 427_14270_472(ATM)_64 | 0 | — |
| chr11 | 108354810 | 108354874 | 427_14271_472(ATM)_65 | 0 | — |
| chr11 | 108365081 | 108365218 | 427_14272_472(ATM)_66 | 0 | — |
| chr11 | 108365324 | 108365508 | 427_14273_472(ATM)_67 | 0 | — |
| chr11 | 118898414 | 118899508 | 427_22416_283149(BCL9L)_10 | 0 | — |
| chr11 | 118899916 | 118900198 | 427_22415_283149(BCL9L)_9 | 0 | — |
| chr11 | 118900618 | 118902908 | 427_22414_283149(BCL9L)_8 | 0 | — |
| chr11 | 118902989 | 118903074 | 427_22413_283149(BCL9L)_7 | 0 | — |
| chr11 | 118903235 | 118903452 | 427_22412_283149(BCL9L)_6 | 0 | — |
| chr11 | 118907482 | 118907602 | 427_22411_283149(BCL9L)_5 | 0 | — |
| chr11 | 118908269 | 118908655 | 427_22410_283149(BCL9L)_4 | 0 | — |
| chr11 | 118909913 | 118909939 | 427_22409_283149(BCL9L)_3 | 0 | — |
| chr12 | 6570675 | 6570693 | 429_33493_1108(CHD4)_40 | 0 | — |
| chr12 | 6570868 | 6571032 | 429_33492_1108(CHD4)_39 | 0 | — |
| chr12 | 6573073 | 6573269 | 429_33491_1108(CHD4)_38 | 0 | — |
| chr12 | 6577784 | 6577917 | 429_33490_1108(CHD4)_37 | 0 | — |
| chr12 | 6578028 | 6578137 | 429_33489_1108(CHD4)_36 | 0 | — |
| chr12 | 6578408 | 6578546 | 429_33488_1108(CHD4)_35 | 0 | — |
| chr12 | 6578845 | 6578917 | 429_33487_1108(CHD4)_34 | 0 | — |
| chr12 | 6581043 | 6581173 | 429_33486_1108(CHD4)_33 | 0 | — |
| chr12 | 6581290 | 6581388 | 429_33485_1108(CHD4)_32 | 0 | — |
| chr12 | 6581648 | 6581814 | 429_33484_1108(CHD4)_31 | 0 | — |
| chr12 | 6582136 | 6582281 | 429_33483_1108(CHD4)_30 | 0 | — |
| chr12 | 6582614 | 6582748 | 429_33482_1108(CHD4)_29 | 0 | — |
| chr12 | 6582847 | 6582936 | 429_33481_1108(CHD4)_28 | 0 | — |
| chr12 | 6583026 | 6583113 | 429_33480_1108(CHD4)_27 | 0 | — |
| chr12 | 6583197 | 6583378 | 429_33479_1108(CHD4)_26 | 0 | — |
| chr12 | 6587383 | 6587559 | 429_33478_1108(CHD4)_25 | 0 | — |
| chr12 | 6587711 | 6587949 | 429_33477_1108(CHD4)_24 | 0 | — |
| chr12 | 6588297 | 6588422 | 429_33476_1108(CHD4)_23 | 0 | — |
| chr12 | 6591465 | 6591583 | 429_33475_1108(CHD4)_22 | 0 | — |
| chr12 | 6591693 | 6591825 | 429_33474_1108(CHD4)_21 | 0 | — |
| chr12 | 6591915 | 6592057 | 429_33473_1108(CHD4)_20 | 0 | — |
| chr12 | 6592392 | 6592566 | 429_33472_1108(CHD4)_19 | 0 | — |
| chr12 | 6592695 | 6592817 | 429_33471_1108(CHD4)_18 | 0 | — |
| chr12 | 6593090 | 6593228 | 429_33470_1108(CHD4)_17 | 0 | — |
| chr12 | 6593415 | 6593616 | 429_33469_1108(CHD4)_16 | 0 | — |
| chr12 | 6594458 | 6594650 | 429_33468_1108(CHD4)_15 | 0 | — |
| chr12 | 6595333 | 6595430 | 429_33467_1108(CHD4)_14 | 0 | — |
| chr12 | 6596005 | 6596137 | 429_33466_1108(CHD4)_13 | 0 | — |
| chr12 | 6597893 | 6598099 | 429_33465_1108(CHD4)_12 | 0 | — |
| chr12 | 6598221 | 6598425 | 429_33464_1108(CHD4)_11 | 0 | — |
| chr12 | 6599772 | 6600012 | 429_33463_1108(CHD4)_10 | 0 | — |
| chr12 | 6600216 | 6600395 | 429_33462_1108(CHD4)_9 | 0 | — |
| chr12 | 6600533 | 6600669 | 429_33461_1108(CHD4)_8 | 0 | — |
| chr12 | 6600925 | 6601053 | 429_33460_1108(CHD4)_7 | 0 | — |
| chr12 | 6601288 | 6601530 | 429_33459_1108(CHD4)_6 | 0 | — |
| chr12 | 6601647 | 6601766 | 429_33458_1108(CHD4)_5 | 0 | — |
| chr12 | 6601959 | 6602175 | 429_33457_1108(CHD4)_4 | 0 | — |
| chr12 | 6602375 | 6602497 | 429_33456_1108(CHD4)_3 | 0 | — |
| chr12 | 6606273 | 6606373 | 429_33455_1108(CHD4)_2 | 0 | — |
| chr12 | 12717839 | 12718314 | 428_25760_1027(CDKN1B)_1a | 0 | — |
| chr12 | 12718824 | 12718946 | 428_25761_1027(CDKN1B)_2a | 0 | — |
| chr12 | 25209510 | 25209911 | 464_89190_3845(KRAS)_6 | 0 | — |
| chr12 | 25215149 | 25215560 | 464_89189_3845(KRAS)_5 | 0 | — |
| chr12 | 25225613 | 25225773 | 464_89188_3845(KRAS)_4 | 0 | — |
| chr12 | 25227233 | 25227412 | 464_89187_3845(KRAS)_3 | 0 | — |
| chr12 | 25245273 | 25245395 | 464_89186_3845(KRAS)_2 | 0 | — |
| chr12 | 56080300 | 56080382 | 461_58650_2065(ERBB3)_1a | 0 | — |
| chr12 | 56080300 | 56080382 | 461_58650_2065(ERBB3)_1a | 0 | — |
| chr12 | 56083750 | 56083902 | 461_58651_2065(ERBB3)_2a | 0 | — |
| chr12 | 56083750 | 56083902 | 461_58651_2065(ERBB3)_2a | 0 | — |
| chr12 | 56084994 | 56085617 | 461_58652_2065(ERBB3)_3a | 0 | — |
| chr12 | 56084994 | 56085617 | 461_58652_2065(ERBB3)_3a | 0 | — |
| chr12 | 56086530 | 56086656 | 461_58653_2065(ERBB3)_4a | 0 | — |
| chr12 | 56086530 | 56086656 | 461_58653_2065(ERBB3)_4a | 0 | — |
| chr12 | 56087576 | 56087642 | 461_58654_2065(ERBB3)_5a | 0 | — |
| chr12 | 56087576 | 56087642 | 461_58654_2065(ERBB3)_5a | 0 | — |
| chr12 | 56087794 | 56087913 | 461_58655_2065(ERBB3)_6a | 0 | — |
| chr12 | 56087794 | 56087913 | 461_58655_2065(ERBB3)_6a | 0 | — |
| chr12 | 56088020 | 56088162 | 461_58656_2065(ERBB3)_7a | 0 | — |
| chr12 | 56088020 | 56088162 | 461_58656_2065(ERBB3)_7a | 0 | — |
| chr12 | 56088542 | 56088656 | 461_58657_2065(ERBB3)_8a | 0 | — |
| chr12 | 56088542 | 56088656 | 461_58657_2065(ERBB3)_8a | 0 | — |
| chr12 | 56088747 | 56088868 | 461_58658_2065(ERBB3)_9a | 0 | — |
| chr12 | 56088747 | 56088868 | 461_58658_2065(ERBB3)_9a | 0 | — |
| chr12 | 56092746 | 56092820 | 461_58659_2065(ERBB3)_10a | 0 | — |
| chr12 | 56092746 | 56092820 | 461_58659_2065(ERBB3)_10a | 0 | — |
| chr12 | 56092985 | 56093076 | 461_58660_2065(ERBB3)_11a | 0 | — |
| chr12 | 56092985 | 56093076 | 461_58660_2065(ERBB3)_11a | 0 | — |
| chr12 | 56093344 | 56093550 | 461_58755_2065(ERBB3)_12a | 0 | — |
| chr12 | 56093344 | 56093550 | 461_58755_2065(ERBB3)_12a | 0 | — |
| chr12 | 56093763 | 56093896 | 461_58756_2065(ERBB3)_13a | 0 | — |
| chr12 | 56093763 | 56093896 | 461_58756_2065(ERBB3)_13a | 0 | — |
| chr12 | 56094098 | 56094189 | 461_58757_2065(ERBB3)_14a | 0 | — |
| chr12 | 56094098 | 56094189 | 461_58757_2065(ERBB3)_14a | 0 | — |
| chr12 | 56094401 | 56094556 | 461_58758_2065(ERBB3)_15a | 0 | — |
| chr12 | 56094401 | 56094556 | 461_58758_2065(ERBB3)_15a | 0 | — |
| chr12 | 56095256 | 56095310 | 461_58759_2065(ERBB3)_16a | 0 | — |
| chr12 | 56095256 | 56095310 | 461_58759_2065(ERBB3)_16a | 0 | — |
| chr12 | 56095664 | 56095806 | 461_58760_2065(ERBB3)_17a | 0 | — |
| chr12 | 56095664 | 56095806 | 461_58760_2065(ERBB3)_17a | 0 | — |
| chr12 | 56096502 | 56096622 | 461_58761_2065(ERBB3)_18a | 0 | — |
| chr12 | 56096502 | 56096622 | 461_58761_2065(ERBB3)_18a | 0 | — |
| chr12 | 56096747 | 56096846 | 461_58762_2065(ERBB3)_19a | 0 | — |
| chr12 | 56096747 | 56096846 | 461_58762_2065(ERBB3)_19a | 0 | — |
| chr12 | 56097044 | 56097230 | 461_58763_2065(ERBB3)_20a | 0 | — |
| chr12 | 56097044 | 56097230 | 461_58763_2065(ERBB3)_20a | 0 | — |
| chr12 | 56097784 | 56097940 | 461_58764_2065(ERBB3)_21a | 0 | — |
| chr12 | 56097784 | 56097940 | 461_58764_2065(ERBB3)_21a | 0 | — |
| chr12 | 56098499 | 56098575 | 461_58765_2065(ERBB3)_22a | 0 | — |
| chr12 | 56098499 | 56098575 | 461_58765_2065(ERBB3)_22a | 0 | — |
| chr12 | 56098758 | 56098905 | 461_58766_2065(ERBB3)_23a | 0 | — |
| chr12 | 56098758 | 56098905 | 461_58766_2065(ERBB3)_23a | 0 | — |
| chr12 | 56099647 | 56099745 | 461_58767_2065(ERBB3)_24a | 0 | — |
| chr12 | 56099647 | 56099745 | 461_58767_2065(ERBB3)_24a | 0 | — |
| chr12 | 56099837 | 56100029 | 461_58768_2065(ERBB3)_25a | 0 | — |
| chr12 | 56099837 | 56100029 | 461_58768_2065(ERBB3)_25a | 0 | — |
| chr12 | 56100173 | 56100245 | 461_58769_2065(ERBB3)_26a | 0 | — |
| chr12 | 56100173 | 56100245 | 461_58769_2065(ERBB3)_26a | 0 | — |
| chr12 | 56101060 | 56101361 | 461_58770_2065(ERBB3)_27a | 0 | — |
| chr12 | 56101060 | 56101361 | 461_58770_2065(ERBB3)_27a | 0 | — |
| chr12 | 56101528 | 56102055 | 461_58771_2065(ERBB3)_28a | 0 | — |
| chr12 | 56101528 | 56102055 | 461_58771_2065(ERBB3)_28a | 0 | — |
| chr12 | 68808477 | 68808491 | 466_106043_4193(MDM2)_1 | 0 | — |
| chr12 | 68808949 | 68809086 | 466_106044_4193(MDM2)_2 | 0 | — |
| chr12 | 68809207 | 68809292 | 466_106045_4193(MDM2)_3 | 0 | — |
| chr12 | 68813553 | 68813628 | 466_106046_4193(MDM2)_4 | 0 | — |
| chr12 | 68816811 | 68816945 | 466_106047_4193(MDM2)_5 | 0 | — |
| chr12 | 68820324 | 68820374 | 466_106048_4193(MDM2)_6 | 0 | — |
| chr12 | 68824362 | 68824430 | 466_106049_4193(MDM2)_7 | 0 | — |
| chr12 | 68824554 | 68824651 | 466_106050_4193(MDM2)_8 | 0 | — |
| chr12 | 68828770 | 68828931 | 466_106051_4193(MDM2)_9 | 0 | — |
| chr12 | 68835828 | 68835984 | 466_106052_4193(MDM2)_10 | 0 | — |
| chr12 | 68836671 | 68836749 | 466_106053_4193(MDM2)_11 | 0 | — |
| chr12 | 68839273 | 68839849 | 466_106054_4193(MDM2)_12 | 0 | — |
| chr12 | 112419111 | 112419125 | 472_140049_5781(PTPN11)_1a | 0 | — |
| chr12 | 112446275 | 112446398 | 472_140050_5781(PTPN11)_2a | 0 | — |
| chr12 | 112450317 | 112450512 | 472_140051_5781(PTPN11)_3a | 0 | — |
| chr12 | 112453194 | 112453387 | 472_140052_5781(PTPN11)_4a | 0 | — |
| chr12 | 112454563 | 112454680 | 472_140053_5781(PTPN11)_5a | 0 | — |
| chr12 | 112455949 | 112456063 | 472_140054_5781(PTPN11)_6a | 0 | — |
| chr12 | 112472943 | 112473040 | 472_140055_5781(PTPN11)_7a | 0 | — |
| chr12 | 112477650 | 112477730 | 472_140056_5781(PTPN11)_8a | 0 | — |
| chr12 | 112477856 | 112478015 | 472_140057_5781(PTPN11)_9a | 0 | — |
| chr12 | 112482073 | 112482205 | 472_140058_5781(PTPN11)_10a | 0 | — |
| chr12 | 112486474 | 112486923 | 472_140059_5781(PTPN11)_11a | 0 | — |
| chr12 | 112488442 | 112488510 | 472_140060_5781(PTPN11)_12a | 0 | — |
| chr12 | 112489023 | 112489175 | 472_140061_5781(PTPN11)_13a | 0 | — |
| chr12 | 112502143 | 112502256 | 472_140062_5781(PTPN11)_14a | 0 | — |
| chr12 | 112504694 | 112504764 | 472_140063_5781(PTPN11)_15a | 0 | — |
| chr12 | 120978768 | 120979094 | 463_81838_6927(HNF1A)_1 | 0 | — |
| chr12 | 120988832 | 120989032 | 463_81839_6927(HNF1A)_2 | 0 | — |
| chr12 | 120993519 | 120993706 | 463_81840_6927(HNF1A)_3 | 0 | — |
| chr12 | 120994163 | 120994405 | 463_81841_6927(HNF1A)_4 | 0 | — |
| chr12 | 120996261 | 120996413 | 463_81842_6927(HNF1A)_5 | 0 | — |
| chr12 | 120996540 | 120997166 | 463_81843_6927(HNF1A)_6 | 0 | — |
| chr12 | 120997473 | 120997665 | 463_81844_6927(HNF1A)_7 | 0 | — |
| chr12 | 120999267 | 120999627 | 463_81845_6927(HNF1A)_8 | 0 | — |
| chr12 | 121001064 | 121001192 | 463_81846_6927(HNF1A)_9 | 0 | — |
| chr12 | 132624696 | 132624810 | 470_129090_5426(POLE)_49 | 0 | — |
| chr12 | 132624904 | 132624994 | 470_129089_5426(POLE)_48 | 0 | — |
| chr12 | 132625644 | 132625770 | 470_129088_5426(POLE)_47 | 0 | — |
| chr12 | 132626116 | 132626317 | 470_129087_5426(POLE)_46 | 0 | — |
| chr12 | 132632314 | 132632508 | 470_129086_5426(POLE)_45 | 0 | — |
| chr12 | 132632663 | 132632795 | 470_129085_5426(POLE)_44 | 0 | — |
| chr12 | 132634185 | 132634378 | 470_129084_5426(POLE)_43 | 0 | — |
| chr12 | 132635891 | 132636024 | 470_129083_5426(POLE)_42 | 0 | — |
| chr12 | 132638013 | 132638139 | 470_129082_5426(POLE)_41 | 0 | — |
| chr12 | 132639124 | 132639298 | 470_129081_5426(POLE)_40 | 0 | — |
| chr12 | 132641646 | 132641851 | 470_129080_5426(POLE)_39 | 0 | — |
| chr12 | 132642176 | 132642397 | 470_129079_5426(POLE)_38 | 0 | — |
| chr12 | 132642505 | 132642729 | 470_129078_5426(POLE)_37 | 0 | — |
| chr12 | 132642819 | 132642996 | 470_129077_5426(POLE)_36 | 0 | — |
| chr12 | 132643223 | 132643330 | 470_129076_5426(POLE)_35 | 0 | — |
| chr12 | 132643406 | 132643560 | 470_129075_5426(POLE)_34 | 0 | — |
| chr12 | 132643836 | 132643977 | 470_129074_5426(POLE)_33 | 0 | — |
| chr12 | 132648928 | 132649072 | 470_129073_5426(POLE)_32 | 0 | — |
| chr12 | 132649305 | 132649515 | 470_129072_5426(POLE)_31 | 0 | — |
| chr12 | 132649676 | 132649889 | 470_129071_5426(POLE)_30 | 0 | — |
| chr12 | 132657135 | 132657258 | 470_129070_5426(POLE)_29 | 0 | — |
| chr12 | 132657348 | 132657429 | 470_129069_5426(POLE)_28 | 0 | — |
| chr12 | 132657867 | 132657970 | 470_129068_5426(POLE)_27 | 0 | — |
| chr12 | 132659294 | 132659509 | 470_129067_5426(POLE)_26 | 0 | — |
| chr12 | 132660968 | 132661164 | 470_129066_5426(POLE)_25 | 0 | — |
| chr12 | 132661526 | 132661684 | 470_129065_5426(POLE)_24 | 0 | — |
| chr12 | 132664003 | 132664148 | 470_129064_5426(POLE)_23 | 0 | — |
| chr12 | 132664369 | 132664462 | 470_129063_5426(POLE)_22 | 0 | — |
| chr12 | 132665301 | 132665450 | 470_129062_5426(POLE)_21 | 0 | — |
| chr12 | 132667502 | 132667648 | 470_129061_5426(POLE)_20 | 0 | — |
| chr12 | 132668355 | 132668502 | 470_129060_5426(POLE)_19 | 0 | — |
| chr12 | 132668634 | 132668737 | 470_129059_5426(POLE)_18 | 0 | — |
| chr12 | 132668810 | 132668939 | 470_129058_5426(POLE)_17 | 0 | — |
| chr12 | 132672214 | 132672322 | 470_129057_5426(POLE)_16 | 0 | — |
| chr12 | 132672626 | 132672839 | 470_129056_5426(POLE)_15 | 0 | — |
| chr12 | 132673163 | 132673277 | 470_129055_5426(POLE)_14 | 0 | — |
| chr12 | 132673574 | 132673707 | 470_129054_5426(POLE)_13 | 0 | — |
| chr12 | 132675397 | 132675517 | 470_129053_5426(POLE)_12 | 0 | — |
| chr12 | 132675734 | 132675820 | 470_129052_5426(POLE)_11 | 0 | — |
| chr12 | 132676093 | 132676204 | 470_129051_5426(POLE)_10 | 0 | — |
| chr12 | 132676545 | 132676653 | 470_129050_5426(POLE)_9 | 0 | — |
| chr12 | 132677362 | 132677443 | 470_129049_5426(POLE)_8 | 0 | — |
| chr12 | 132677577 | 132677719 | 470_129048_5426(POLE)_7 | 0 | — |
| chr12 | 132679496 | 132679651 | 470_129047_5426(POLE)_6 | 0 | — |
| chr12 | 132679953 | 132680046 | 470_129046_5426(POLE)_5 | 0 | — |
| chr12 | 132680177 | 132680222 | 470_129045_5426(POLE)_4 | 0 | — |
| chr12 | 132680606 | 132680687 | 470_129044_5426(POLE)_3 | 0 | — |
| chr12 | 132681137 | 132681279 | 470_129043_5426(POLE)_2 | 0 | — |
| chr12 | 132687253 | 132687315 | 470_129042_5426(POLE)_1 | 0 | — |
| chr13 | 28004051 | 28004174 | 461_60876_2322(FLT3)_25a | 0 | — |
| chr13 | 28014451 | 28014557 | 461_60875_2322(FLT3)_24a | 0 | — |
| chr13 | 28015156 | 28015256 | 461_60874_2322(FLT3)_23a | 0 | — |
| chr13 | 28015589 | 28015701 | 461_60873_2322(FLT3)_22a | 0 | — |
| chr13 | 28018466 | 28018589 | 461_60872_2322(FLT3)_21a | 0 | — |
| chr13 | 28023349 | 28023477 | 461_60871_2322(FLT3)_20a | 0 | — |
| chr13 | 28024860 | 28024943 | 461_60870_2322(FLT3)_19a | 0 | — |
| chr13 | 28027087 | 28027241 | 461_60869_2322(FLT3)_18a | 0 | — |
| chr13 | 28028177 | 28028288 | 461_60868_2322(FLT3)_17a | 0 | — |
| chr13 | 28033886 | 28033991 | 461_60867_2322(FLT3)_16a | 0 | — |
| chr13 | 28034081 | 28034214 | 461_60866_2322(FLT3)_15a | 0 | — |
| chr13 | 28034300 | 28034407 | 461_60865_2322(FLT3)_14a | 0 | — |
| chr13 | 28035494 | 28035673 | 461_60864_2322(FLT3)_13a | 0 | — |
| chr13 | 28035934 | 28036043 | 461_60863_2322(FLT3)_12a | 0 | — |
| chr13 | 28037184 | 28037288 | 461_60862_2322(FLT3)_11a | 0 | — |
| chr13 | 28048274 | 28048443 | 461_60861_2322(FLT3)_10a | 0 | — |
| chr13 | 28049383 | 28049537 | 461_60860_2322(FLT3)_9a | 0 | — |
| chr13 | 28049634 | 28049774 | 461_60859_2322(FLT3)_8a | 0 | — |
| chr13 | 28050094 | 28050222 | 461_60858_2322(FLT3)_7a | 0 | — |
| chr13 | 28052544 | 28052674 | 461_60857_2322(FLT3)_6a | 0 | — |
| chr13 | 28057346 | 28057462 | 461_60856_2322(FLT3)_5a | 0 | — |
| chr13 | 28061866 | 28062069 | 461_60855_2322(FLT3)_4a | 0 | — |
| chr13 | 28070490 | 28070612 | 461_60854_2322(FLT3)_3a | 0 | — |
| chr13 | 28100467 | 28100510 | 461_60853_2322(FLT3)_2a | 0 | — |
| chr13 | 32316460 | 32316527 | 427_15650_675(BRCA2)_2 | 0 | — |
| chr13 | 32319076 | 32319325 | 427_15651_675(BRCA2)_3 | 0 | — |
| chr13 | 32325075 | 32325184 | 427_15652_675(BRCA2)_4 | 0 | — |
| chr13 | 32326100 | 32326150 | 427_15653_675(BRCA2)_5 | 0 | — |
| chr13 | 32326241 | 32326282 | 427_15654_675(BRCA2)_6 | 0 | — |
| chr13 | 32326498 | 32326613 | 427_15655_675(BRCA2)_7 | 0 | — |
| chr13 | 32329442 | 32329492 | 427_15656_675(BRCA2)_8 | 0 | — |
| chr13 | 32330918 | 32331030 | 427_15657_675(BRCA2)_9 | 0 | — |
| chr13 | 32332271 | 32333387 | 427_15658_675(BRCA2)_10 | 0 | — |
| chr13 | 32336264 | 32341196 | 427_15659_675(BRCA2)_11 | 0 | — |
| chr13 | 32344557 | 32344653 | 427_15660_675(BRCA2)_12 | 0 | — |
| chr13 | 32346826 | 32346896 | 427_15661_675(BRCA2)_13 | 0 | — |
| chr13 | 32354897 | 32355325 | 427_15662_675(BRCA2)_14 | 0 | — |
| chr13 | 32356427 | 32356609 | 427_15663_675(BRCA2)_15 | 0 | — |
| chr13 | 32357741 | 32357929 | 427_15664_675(BRCA2)_16 | 0 | — |
| chr13 | 32362522 | 32362693 | 427_15665_675(BRCA2)_17 | 0 | — |
| chr13 | 32363178 | 32363533 | 427_15666_675(BRCA2)_18 | 0 | — |
| chr13 | 32370401 | 32370557 | 427_15667_675(BRCA2)_19 | 0 | — |
| chr13 | 32370955 | 32371100 | 427_15668_675(BRCA2)_20 | 0 | — |
| chr13 | 32376669 | 32376791 | 427_15669_675(BRCA2)_21 | 0 | — |
| chr13 | 32379316 | 32379515 | 427_15670_675(BRCA2)_22 | 0 | — |
| chr13 | 32379749 | 32379913 | 427_15671_675(BRCA2)_23 | 0 | — |
| chr13 | 32380006 | 32380145 | 427_15672_675(BRCA2)_24 | 0 | — |
| chr13 | 32394688 | 32394933 | 427_15673_675(BRCA2)_25 | 0 | — |
| chr13 | 32396897 | 32397044 | 427_15674_675(BRCA2)_26 | 0 | — |
| chr13 | 32398161 | 32398770 | 427_15675_675(BRCA2)_27 | 0 | — |
| chr13 | 48303912 | 48304049 | 472_141443_5925(RB1)_1a | 0 | — |
| chr13 | 48307279 | 48307406 | 472_141444_5925(RB1)_2a | 0 | — |
| chr13 | 48342598 | 48342714 | 472_141445_5925(RB1)_3a | 0 | — |
| chr13 | 48345079 | 48345199 | 472_141446_5925(RB1)_4a | 0 | — |
| chr13 | 48347824 | 48347863 | 472_141447_5925(RB1)_5a | 0 | — |
| chr13 | 48348955 | 48349023 | 472_141448_5925(RB1)_6a | 0 | — |
| chr13 | 48360016 | 48360127 | 472_141449_5925(RB1)_7a | 0 | — |
| chr13 | 48362814 | 48362957 | 472_141450_5925(RB1)_8a | 0 | — |
| chr13 | 48364893 | 48364971 | 472_141451_5925(RB1)_9a | 0 | — |
| chr13 | 48367493 | 48367603 | 472_141452_5925(RB1)_10a | 0 | — |
| chr13 | 48368526 | 48368604 | 472_141453_5925(RB1)_11a | 0 | — |
| chr13 | 48373404 | 48373492 | 472_141454_5925(RB1)_12a | 0 | — |
| chr13 | 48376917 | 48377034 | 472_141455_5925(RB1)_13a | 0 | — |
| chr13 | 48379593 | 48379650 | 472_141456_5925(RB1)_14a | 0 | — |
| chr13 | 48380052 | 48380084 | 472_141457_5925(RB1)_15a | 0 | — |
| chr13 | 48380164 | 48380241 | 472_141458_5925(RB1)_16a | 0 | — |
| chr13 | 48381246 | 48381443 | 472_141459_5925(RB1)_17a | 0 | — |
| chr13 | 48452992 | 48453111 | 472_141460_5925(RB1)_18a | 0 | — |
| chr13 | 48456203 | 48456349 | 472_141461_5925(RB1)_19a | 0 | — |
| chr13 | 48459687 | 48459833 | 472_141462_5925(RB1)_20a | 0 | — |
| chr13 | 48463730 | 48463835 | 472_141463_5925(RB1)_21a | 0 | — |
| chr13 | 48464997 | 48465111 | 472_141464_5925(RB1)_22a | 0 | — |
| chr13 | 48465204 | 48465368 | 472_141465_5925(RB1)_23a | 0 | — |
| chr13 | 48473359 | 48473390 | 472_141466_5925(RB1)_24a | 0 | — |
| chr13 | 48476700 | 48476843 | 472_141467_5925(RB1)_25a | 0 | — |
| chr13 | 48477354 | 48477404 | 472_141468_5925(RB1)_26a | 0 | — |
| chr13 | 48479997 | 48480071 | 472_141469_5925(RB1)_27a | 0 | — |
| chr14 | 104770340 | 104770420 | 426_1783_207(AKT1)_17a | 0 | — |
| chr14 | 104770744 | 104770847 | 426_1782_207(AKT1)_16a | 0 | — |
| chr14 | 104772364 | 104772452 | 426_1781_207(AKT1)_15a | 0 | — |
| chr14 | 104772877 | 104773092 | 426_1780_207(AKT1)_14a | 0 | — |
| chr14 | 104773250 | 104773379 | 426_1779_207(AKT1)_13a | 0 | — |
| chr14 | 104773454 | 104773580 | 426_1778_207(AKT1)_12a | 0 | — |
| chr14 | 104773911 | 104773980 | 426_1777_207(AKT1)_11a | 0 | — |
| chr14 | 104774937 | 104775003 | 426_1776_207(AKT1)_10a | 0 | — |
| chr14 | 104775075 | 104775207 | 426_1775_207(AKT1)_9a | 0 | — |
| chr14 | 104775651 | 104775799 | 426_1774_207(AKT1)_8a | 0 | — |
| chr14 | 104776658 | 104776770 | 426_1773_207(AKT1)_7a | 0 | — |
| chr14 | 104777568 | 104777740 | 426_1772_207(AKT1)_6a | 0 | — |
| chr14 | 104780087 | 104780216 | 426_1771_207(AKT1)_5a | 0 | — |
| chr14 | 104792597 | 104792643 | 426_1770_207(AKT1)_4a | 0 | — |
| chr15 | 43746539 | 43746706 | 470_105606_2923(PDIA3)_1 | 0 | — |
| chr15 | 43751588 | 43751758 | 470_105607_2923(PDIA3)_2 | 0 | — |
| chr15 | 43753823 | 43753902 | 470_105608_2923(PDIA3)_3 | 0 | — |
| chr15 | 43756648 | 43756766 | 470_125487_2923(PDIA3)_4 | 0 | — |
| chr15 | 43761423 | 43761531 | 470_125488_2923(PDIA3)_5 | 0 | — |
| chr15 | 43763076 | 43763206 | 470_125489_2923(PDIA3)_6 | 0 | — |
| chr15 | 43765449 | 43765566 | 470_125490_2923(PDIA3)_7 | 0 | — |
| chr15 | 43765886 | 43766012 | 470_125491_2923(PDIA3)_8 | 0 | — |
| chr15 | 43766727 | 43766910 | 470_125492_2923(PDIA3)_9 | 0 | — |
| chr15 | 43768488 | 43768597 | 470_125493_2923(PDIA3)_10 | 0 | — |
| chr15 | 43769517 | 43769646 | 470_125494_2923(PDIA3)_11 | 0 | — |
| chr15 | 43770249 | 43770329 | 470_125495_2923(PDIA3)_12 | 0 | — |
| chr15 | 43770522 | 43770580 | 470_125496_2923(PDIA3)_13 | 0 | — |
| chr15 | 43771104 | 43771218 | 470_125497_2923(PDIA3)_14 | 0 | — |
| chr15 | 44711541 | 44711613 | 427_14957_567(B2M)_1 | 0 | — |
| chr15 | 44715417 | 44715719 | 427_14958_567(B2M)_2 | 0 | — |
| chr15 | 44716328 | 44716342 | 427_14959_567(B2M)_3 | 0 | — |
| chr15 | 66387347 | 66387427 | 465_99849_5604(MAP2K1)_1a | 0 | — |
| chr15 | 66401862 | 66401947 | 465_99850_5604(MAP2K1)_2a | 0 | — |
| chr15 | 66435026 | 66435237 | 465_99851_5604(MAP2K1)_3a | 0 | — |
| chr15 | 66436745 | 66436892 | 465_99852_5604(MAP2K1)_4a | 0 | — |
| chr15 | 66443279 | 66443357 | 465_99853_5604(MAP2K1)_5a | 0 | — |
| chr15 | 66444655 | 66444707 | 465_99854_5604(MAP2K1)_6a | 0 | — |
| chr15 | 66481754 | 66481879 | 465_99855_5604(MAP2K1)_7a | 0 | — |
| chr15 | 66484989 | 66485191 | 465_99856_5604(MAP2K1)_8a | 0 | — |
| chr15 | 66487227 | 66487292 | 465_99857_5604(MAP2K1)_9a | 0 | — |
| chr15 | 66489214 | 66489276 | 465_99858_5604(MAP2K1)_10a | 0 | — |
| chr15 | 66489717 | 66489763 | 465_99859_5604(MAP2K1)_11a | 0 | — |
| chr15 | 66490501 | 66490615 | 465_99860_5604(MAP2K1)_12a | 0 | — |
| chr15 | 90084265 | 90084353 | 463_79511_3418(IDH2)_12a | 0 | — |
| chr15 | 90084815 | 90084908 | 463_79510_3418(IDH2)_11a | 0 | — |
| chr15 | 90085000 | 90085098 | 463_79509_3418(IDH2)_10a | 0 | — |
| chr15 | 90085274 | 90085387 | 463_79508_3418(IDH2)_9a | 0 | — |
| chr15 | 90087111 | 90087263 | 463_79507_3418(IDH2)_8a | 0 | — |
| chr15 | 90087438 | 90087575 | 463_79506_3418(IDH2)_7a | 0 | — |
| chr15 | 90088358 | 90088502 | 463_79505_3418(IDH2)_6a | 0 | — |
| chr15 | 90088586 | 90088747 | 463_79504_3418(IDH2)_5a | 0 | — |
| chr15 | 90090478 | 90090644 | 463_79503_3418(IDH2)_4a | 0 | — |
| chr15 | 90091552 | 90091644 | 463_79502_3418(IDH2)_3a | 0 | — |
| chr15 | 90100575 | 90100690 | 463_79501_3418(IDH2)_2a | 0 | — |
| chr15 | 90102275 | 90102390 | 463_79500_3418(IDH2)_1a | 0 | — |
| chr16 | 2047821 | 2048065 | 476_182338_7249(TSC2)_1 | 0 | — |
| chr16 | 2048586 | 2048753 | 476_182339_7249(TSC2)_2 | 0 | — |
| chr16 | 2050399 | 2050486 | 476_182340_7249(TSC2)_3 | 0 | — |
| chr16 | 2053341 | 2053452 | 476_182341_7249(TSC2)_4 | 0 | — |
| chr16 | 2054295 | 2054440 | 476_182342_7249(TSC2)_5 | 0 | — |
| chr16 | 2055401 | 2055519 | 476_182343_7249(TSC2)_6 | 0 | — |
| chr16 | 2056195 | 2056244 | 476_182344_7249(TSC2)_7 | 0 | — |
| chr16 | 2056643 | 2056769 | 476_182345_7249(TSC2)_8 | 0 | — |
| chr16 | 2057104 | 2057178 | 476_182346_7249(TSC2)_9 | 0 | — |
| chr16 | 2058746 | 2058873 | 476_182347_7249(TSC2)_10 | 0 | — |
| chr16 | 2060669 | 2060813 | 476_182348_7249(TSC2)_11 | 0 | — |
| chr16 | 2061870 | 2062008 | 476_182349_7249(TSC2)_12 | 0 | — |
| chr16 | 2062496 | 2062600 | 476_182350_7249(TSC2)_13 | 0 | — |
| chr16 | 2062971 | 2063053 | 476_182351_7249(TSC2)_14 | 0 | — |
| chr16 | 2064271 | 2064427 | 476_182352_7249(TSC2)_15 | 0 | — |
| chr16 | 2065518 | 2065635 | 476_182353_7249(TSC2)_16 | 0 | — |
| chr16 | 2070455 | 2070578 | 476_182354_7249(TSC2)_17 | 0 | — |
| chr16 | 2071509 | 2071616 | 476_182355_7249(TSC2)_18 | 0 | — |
| chr16 | 2071783 | 2071934 | 476_182356_7249(TSC2)_19 | 0 | — |
| chr16 | 2072240 | 2072363 | 476_182357_7249(TSC2)_20 | 0 | — |
| chr16 | 2072848 | 2072983 | 476_182358_7249(TSC2)_21 | 0 | — |
| chr16 | 2074199 | 2074389 | 476_182359_7249(TSC2)_22 | 0 | — |
| chr16 | 2075798 | 2075892 | 476_182360_7249(TSC2)_23 | 0 | — |
| chr16 | 2076067 | 2076257 | 476_182361_7249(TSC2)_24 | 0 | — |
| chr16 | 2076490 | 2076585 | 476_182362_7249(TSC2)_25 | 0 | — |
| chr16 | 2077597 | 2077726 | 476_182363_7249(TSC2)_26 | 0 | — |
| chr16 | 2079031 | 2079196 | 476_182364_7249(TSC2)_27 | 0 | — |
| chr16 | 2079275 | 2079428 | 476_182365_7249(TSC2)_28 | 0 | — |
| chr16 | 2079556 | 2079669 | 476_182366_7249(TSC2)_29 | 0 | — |
| chr16 | 2080164 | 2080377 | 476_182367_7249(TSC2)_30 | 0 | — |
| chr16 | 2081594 | 2081798 | 476_182368_7249(TSC2)_31 | 0 | — |
| chr16 | 2082435 | 2082504 | 476_182369_7249(TSC2)_32 | 0 | — |
| chr16 | 2083694 | 2083816 | 476_182370_7249(TSC2)_33 | 0 | — |
| chr16 | 2084227 | 2084715 | 476_182371_7249(TSC2)_34 | 0 | — |
| chr16 | 2084950 | 2085026 | 476_182372_7249(TSC2)_35 | 0 | — |
| chr16 | 2085229 | 2085322 | 476_182373_7249(TSC2)_36 | 0 | — |
| chr16 | 2086192 | 2086379 | 476_182374_7249(TSC2)_37 | 0 | — |
| chr16 | 2086731 | 2086871 | 476_182375_7249(TSC2)_38 | 0 | — |
| chr16 | 2087862 | 2087941 | 476_182376_7249(TSC2)_39 | 0 | — |
| chr16 | 2088047 | 2088139 | 476_182377_7249(TSC2)_40 | 0 | — |
| chr16 | 2088226 | 2088325 | 476_182378_7249(TSC2)_41 | 0 | — |
| chr16 | 2088445 | 2088610 | 476_182379_7249(TSC2)_42 | 0 | — |
| chr16 | 67610832 | 67611613 | 429_38979_10664(CTCF)_3 | 0 | — |
| chr16 | 67611950 | 67612121 | 429_38980_10664(CTCF)_4 | 0 | — |
| chr16 | 67616744 | 67616878 | 429_38981_10664(CTCF)_5 | 0 | — |
| chr16 | 67620696 | 67620817 | 429_38982_10664(CTCF)_6 | 0 | — |
| chr16 | 67621441 | 67621591 | 429_38983_10664(CTCF)_7 | 0 | — |
| chr16 | 67626554 | 67626715 | 429_38984_10664(CTCF)_8 | 0 | — |
| chr16 | 67628369 | 67628552 | 429_38985_10664(CTCF)_9 | 0 | — |
| chr16 | 67629397 | 67629533 | 429_38986_10664(CTCF)_10 | 0 | — |
| chr16 | 67636689 | 67636851 | 429_38987_10664(CTCF)_11 | 0 | — |
| chr16 | 67637687 | 67637872 | 429_38988_10664(CTCF)_12 | 0 | — |
| chr16 | 68737415 | 68737463 | 428_25420_999(CDH1)_1 | 0 | — |
| chr16 | 68738296 | 68738411 | 428_25421_999(CDH1)_2 | 0 | — |
| chr16 | 68801669 | 68801893 | 428_25422_999(CDH1)_3 | 0 | — |
| chr16 | 68808423 | 68808567 | 428_25423_999(CDH1)_4 | 0 | — |
| chr16 | 68808692 | 68808848 | 428_25424_999(CDH1)_5 | 0 | — |
| chr16 | 68810196 | 68810341 | 428_25425_999(CDH1)_6 | 0 | — |
| chr16 | 68811683 | 68811859 | 428_25426_999(CDH1)_7 | 0 | — |
| chr16 | 68812134 | 68812263 | 428_25427_999(CDH1)_8 | 0 | — |
| chr16 | 68813312 | 68813495 | 428_25428_999(CDH1)_9 | 0 | — |
| chr16 | 68815514 | 68815759 | 428_25429_999(CDH1)_10 | 0 | — |
| chr16 | 68819279 | 68819425 | 428_25430_999(CDH1)_11 | 0 | — |
| chr16 | 68822000 | 68822225 | 428_25431_999(CDH1)_12 | 0 | — |
| chr16 | 68823398 | 68823626 | 428_25432_999(CDH1)_13 | 0 | — |
| chr16 | 68828173 | 68828304 | 428_25433_999(CDH1)_14 | 0 | — |
| chr16 | 68829653 | 68829797 | 428_25434_999(CDH1)_15 | 0 | — |
| chr16 | 68833289 | 68833499 | 428_25435_999(CDH1)_16 | 0 | — |
| chr17 | 7669608 | 7669690 | 476_181712_7157(TP53)_11 | 0 | — |
| chr17 | 7670608 | 7670715 | 476_181711_7157(TP53)_10 | 0 | — |
| chr17 | 7673206 | 7673339 | 476_181710_7157(TP53)_9 | 0 | — |
| chr17 | 7673534 | 7673608 | 476_181709_7157(TP53)_8 | 0 | — |
| chr17 | 7673700 | 7673837 | 476_181708_7157(TP53)_7 | 0 | — |
| chr17 | 7674180 | 7674290 | 476_181707_7157(TP53)_6 | 0 | — |
| chr17 | 7674858 | 7674971 | 476_181706_7157(TP53)_5 | 0 | — |
| chr17 | 7675052 | 7675493 | 476_181705_7157(TP53)_4 | 0 | — |
| chr17 | 7675993 | 7676272 | 476_181704_7157(TP53)_3 | 0 | — |
| chr17 | 7676381 | 7676594 | 476_181703_7157(TP53)_2 | 0 | — |
| chr17 | 12020886 | 12021001 | 465_99947_6416(MAP2K4)_1 | 0 | — |
| chr17 | 12032265 | 12032298 | 465_99948_6416(MAP2K4)_2 | 0 | — |
| chr17 | 12054888 | 12054991 | 465_99949_6416(MAP2K4)_3 | 0 | — |
| chr17 | 12069723 | 12069849 | 465_99950_6416(MAP2K4)_4 | 0 | — |
| chr17 | 12081355 | 12081530 | 465_99951_6416(MAP2K4)_5 | 0 | — |
| chr17 | 12095574 | 12095694 | 465_99952_6416(MAP2K4)_6 | 0 | — |
| chr17 | 12107789 | 12107909 | 465_99953_6416(MAP2K4)_7 | 0 | — |
| chr17 | 12110374 | 12110426 | 465_99954_6416(MAP2K4)_8 | 0 | — |
| chr17 | 12113232 | 12113360 | 465_99955_6416(MAP2K4)_9 | 0 | — |
| chr17 | 12125293 | 12125371 | 465_99956_6416(MAP2K4)_10 | 0 | — |
| chr17 | 12129138 | 12129287 | 465_99957_6416(MAP2K4)_11 | 0 | — |
| chr17 | 12139838 | 12139884 | 465_99958_6416(MAP2K4)_12 | 0 | — |
| chr17 | 12141146 | 12141260 | 465_99959_6416(MAP2K4)_13 | 0 | — |
| chr17 | 31095309 | 31095369 | 469_118574_4763(NF1)_1a | 0 | — |
| chr17 | 31155982 | 31156126 | 469_118575_4763(NF1)_2a | 0 | — |
| chr17 | 31159009 | 31159093 | 469_118576_4763(NF1)_3a | 0 | — |
| chr17 | 31163185 | 31163376 | 469_118577_4763(NF1)_4a | 0 | — |
| chr17 | 31169890 | 31169997 | 469_118578_4763(NF1)_5a | 0 | — |
| chr17 | 31181421 | 31181489 | 469_118579_4763(NF1)_6a | 0 | — |
| chr17 | 31181709 | 31181785 | 469_118580_4763(NF1)_7a | 0 | — |
| chr17 | 31182507 | 31182665 | 469_118581_4763(NF1)_8a | 0 | — |
| chr17 | 31200421 | 31200595 | 469_118582_4763(NF1)_9a | 0 | — |
| chr17 | 31201036 | 31201159 | 469_118583_4763(NF1)_10a | 0 | — |
| chr17 | 31201410 | 31201485 | 469_118584_4763(NF1)_11a | 0 | — |
| chr17 | 31206239 | 31206371 | 469_118585_4763(NF1)_12a | 0 | — |
| chr17 | 31214450 | 31214585 | 469_118586_4763(NF1)_13a | 0 | — |
| chr17 | 31219004 | 31219118 | 469_118587_4763(NF1)_14a | 0 | — |
| chr17 | 31221849 | 31222764 | 469_118588_4763(NF1)_15a | 0 | — |
| chr17 | 31223443 | 31223567 | 469_118589_4763(NF1)_16a | 0 | — |
| chr17 | 31225094 | 31225250 | 469_118590_4763(NF1)_17a | 0 | — |
| chr17 | 31226434 | 31226684 | 469_118591_4763(NF1)_18a | 0 | — |
| chr17 | 31227217 | 31227291 | 469_118592_4763(NF1)_19a | 0 | — |
| chr17 | 31227522 | 31227606 | 469_118593_4763(NF1)_20a | 0 | — |
| chr17 | 31229024 | 31229465 | 469_118594_4763(NF1)_21a | 0 | — |
| chr17 | 31229834 | 31229974 | 469_118595_4763(NF1)_22a | 0 | — |
| chr17 | 31230259 | 31230382 | 469_118596_4763(NF1)_23a | 0 | — |
| chr17 | 31230841 | 31230925 | 469_118597_4763(NF1)_24a | 0 | — |
| chr17 | 31232072 | 31232189 | 469_118598_4763(NF1)_25a | 0 | — |
| chr17 | 31232699 | 31232881 | 469_118599_4763(NF1)_26a | 0 | — |
| chr17 | 31233001 | 31233213 | 469_118600_4763(NF1)_27a | 0 | — |
| chr17 | 31235610 | 31235772 | 469_118601_4763(NF1)_28a | 0 | — |
| chr17 | 31235917 | 31236021 | 469_118602_4763(NF1)_29a | 0 | — |
| chr17 | 31248983 | 31249119 | 469_118603_4763(NF1)_30a | 0 | — |
| chr17 | 31252937 | 31253000 | 469_118604_4763(NF1)_31a | 0 | — |
| chr17 | 31258343 | 31258502 | 469_118605_4763(NF1)_32a | 0 | — |
| chr17 | 31259031 | 31259129 | 469_118606_4763(NF1)_33a | 0 | — |
| chr17 | 31260368 | 31260515 | 469_118607_4763(NF1)_34a | 0 | — |
| chr17 | 31261710 | 31261857 | 469_118608_4763(NF1)_35a | 0 | — |
| chr17 | 31265228 | 31265339 | 469_118609_4763(NF1)_36a | 0 | — |
| chr17 | 31325819 | 31326252 | 469_118610_4763(NF1)_37a | 0 | — |
| chr17 | 31327498 | 31327839 | 469_118611_4763(NF1)_38a | 0 | — |
| chr17 | 31330295 | 31330498 | 469_118612_4763(NF1)_39a | 0 | — |
| chr17 | 31334837 | 31335031 | 469_118613_4763(NF1)_40a | 0 | — |
| chr17 | 31336332 | 31336473 | 469_118614_4763(NF1)_41a | 0 | — |
| chr17 | 31336634 | 31336914 | 469_118615_4763(NF1)_42a | 0 | — |
| chr17 | 31337367 | 31337582 | 469_118616_4763(NF1)_43a | 0 | — |
| chr17 | 31337818 | 31337880 | 469_118617_4763(NF1)_44a | 0 | — |
| chr17 | 31338024 | 31338139 | 469_118618_4763(NF1)_45a | 0 | — |
| chr17 | 31338703 | 31338805 | 469_118619_4763(NF1)_46a | 0 | — |
| chr17 | 31340504 | 31340645 | 469_118620_4763(NF1)_47a | 0 | — |
| chr17 | 31343008 | 31343135 | 469_118621_4763(NF1)_48a | 0 | — |
| chr17 | 31349119 | 31349251 | 469_118622_4763(NF1)_49a | 0 | — |
| chr17 | 31350182 | 31350318 | 469_118623_4763(NF1)_50a | 0 | — |
| chr17 | 31352256 | 31352414 | 469_118624_4763(NF1)_51a | 0 | — |
| chr17 | 31356459 | 31356582 | 469_118625_4763(NF1)_52a | 0 | — |
| chr17 | 31356959 | 31357090 | 469_118626_4763(NF1)_53a | 0 | — |
| chr17 | 31357268 | 31357369 | 469_118627_4763(NF1)_54a | 0 | — |
| chr17 | 31358479 | 31358622 | 469_118628_4763(NF1)_55a | 0 | — |
| chr17 | 31358968 | 31359015 | 469_118629_4763(NF1)_56a | 0 | — |
| chr17 | 31360486 | 31360703 | 469_118630_4763(NF1)_57a | 0 | — |
| chr17 | 31362287 | 31362364 | 469_118631_4763(NF1)_58a | 0 | — |
| chr17 | 31367224 | 31367278 | 469_118632_4763(NF1)_59a | 0 | — |
| chr17 | 31374012 | 31374155 | 469_118633_4763(NF1)_60a | 0 | — |
| chr17 | 39699559 | 39699587 | 461_58727_2064(ERBB2)_5a | 0 | — |
| chr17 | 39700000 | 39700311 | 461_58728_2064(ERBB2)_6a | 0 | — |
| chr17 | 39706987 | 39707141 | 461_58729_2064(ERBB2)_7a | 0 | — |
| chr17 | 39708320 | 39708534 | 461_58730_2064(ERBB2)_8a | 0 | — |
| chr17 | 39709317 | 39709452 | 461_58731_2064(ERBB2)_9a | 0 | — |
| chr17 | 39709812 | 39709881 | 461_58732_2064(ERBB2)_10a | 0 | — |
| chr17 | 39710085 | 39710201 | 461_58733_2064(ERBB2)_11a | 0 | — |
| chr17 | 39710339 | 39710481 | 461_58734_2064(ERBB2)_12a | 0 | — |
| chr17 | 39711927 | 39712047 | 461_58735_2064(ERBB2)_13a | 0 | — |
| chr17 | 39712321 | 39712448 | 461_58736_2064(ERBB2)_14a | 0 | — |
| chr17 | 39715285 | 39715359 | 461_58737_2064(ERBB2)_15a | 0 | — |
| chr17 | 39715445 | 39715536 | 461_58738_2064(ERBB2)_16a | 0 | — |
| chr17 | 39715739 | 39715939 | 461_58739_2064(ERBB2)_17a | 0 | — |
| chr17 | 39716300 | 39716433 | 461_58740_2064(ERBB2)_18a | 0 | — |
| chr17 | 39716514 | 39716605 | 461_58741_2064(ERBB2)_19a | 0 | — |
| chr17 | 39717319 | 39717480 | 461_58742_2064(ERBB2)_20a | 0 | — |
| chr17 | 39719786 | 39719834 | 461_58743_2064(ERBB2)_21a | 0 | — |
| chr17 | 39723318 | 39723457 | 461_58744_2064(ERBB2)_22a | 0 | — |
| chr17 | 39723537 | 39723660 | 461_58745_2064(ERBB2)_23a | 0 | — |
| chr17 | 39723911 | 39724010 | 461_58746_2064(ERBB2)_24a | 0 | — |
| chr17 | 39724725 | 39724911 | 461_58747_2064(ERBB2)_25a | 0 | — |
| chr17 | 39725048 | 39725204 | 461_58748_2064(ERBB2)_26a | 0 | — |
| chr17 | 39725326 | 39725402 | 461_58749_2064(ERBB2)_27a | 0 | — |
| chr17 | 39725706 | 39725853 | 461_58750_2064(ERBB2)_28a | 0 | — |
| chr17 | 39726561 | 39726659 | 461_58751_2064(ERBB2)_29a | 0 | — |
| chr17 | 39726814 | 39727003 | 461_58752_2064(ERBB2)_30a | 0 | — |
| chr17 | 39727294 | 39727547 | 461_58753_2064(ERBB2)_31a | 0 | — |
| chr17 | 39727688 | 39728044 | 461_58754_2064(ERBB2)_32a | 0 | — |
| chr17 | 43045677 | 43045802 | 427_15629_672(BRCA1)_24 | 0 | — |
| chr17 | 43047642 | 43047703 | 427_15628_672(BRCA1)_23 | 0 | — |
| chr17 | 43049120 | 43049194 | 427_15627_672(BRCA1)_22 | 0 | — |
| chr17 | 43051062 | 43051117 | 427_15626_672(BRCA1)_21 | 0 | — |
| chr17 | 43057051 | 43057135 | 427_15625_672(BRCA1)_20 | 0 | — |
| chr17 | 43063332 | 43063373 | 427_15624_672(BRCA1)_19 | 0 | — |
| chr17 | 43063873 | 43063951 | 427_15623_672(BRCA1)_18 | 0 | — |
| chr17 | 43067607 | 43067695 | 427_15622_672(BRCA1)_17 | 0 | — |
| chr17 | 43070927 | 43071238 | 427_15621_672(BRCA1)_16 | 0 | — |
| chr17 | 43074330 | 43074521 | 427_15620_672(BRCA1)_15 | 0 | — |
| chr17 | 43076487 | 43076614 | 427_15619_672(BRCA1)_14 | 0 | — |
| chr17 | 43079333 | 43079399 | 427_15618_672(BRCA1)_13 | 0 | — |
| chr17 | 43082403 | 43082575 | 427_15617_672(BRCA1)_12 | 0 | — |
| chr17 | 43090943 | 43091032 | 427_15616_672(BRCA1)_11 | 0 | — |
| chr17 | 43091434 | 43094860 | 427_15615_672(BRCA1)_10 | 0 | — |
| chr17 | 43095845 | 43095922 | 427_15614_672(BRCA1)_9 | 0 | — |
| chr17 | 43097243 | 43097289 | 427_15613_672(BRCA1)_8 | 0 | — |
| chr17 | 43099774 | 43099880 | 427_15612_672(BRCA1)_7 | 0 | — |
| chr17 | 43104121 | 43104261 | 427_15611_672(BRCA1)_6 | 0 | — |
| chr17 | 43104867 | 43104956 | 427_15610_672(BRCA1)_5 | 0 | — |
| chr17 | 43106455 | 43106533 | 427_15609_672(BRCA1)_4 | 0 | — |
| chr17 | 43115725 | 43115779 | 427_15608_672(BRCA1)_3 | 0 | — |
| chr17 | 43124016 | 43124096 | 427_15607_672(BRCA1)_2 | 0 | — |
| chr17 | 58354942 | 58354986 | 473_154862_54894(RNF43)_10 | 0 | — |
| chr17 | 58357467 | 58358823 | 473_154861_54894(RNF43)_9 | 0 | — |
| chr17 | 58360148 | 58360251 | 473_154860_54894(RNF43)_8 | 0 | — |
| chr17 | 58360782 | 58360944 | 473_154859_54894(RNF43)_7 | 0 | — |
| chr17 | 58362543 | 58362648 | 473_154858_54894(RNF43)_6 | 0 | — |
| chr17 | 58363274 | 58363406 | 473_154857_54894(RNF43)_5 | 0 | — |
| chr17 | 58363525 | 58363600 | 473_154856_54894(RNF43)_4 | 0 | — |
| chr17 | 58370910 | 58371033 | 473_154855_54894(RNF43)_3 | 0 | — |
| chr17 | 58415325 | 58415577 | 473_154854_54894(RNF43)_2 | 0 | — |
| chr17 | 72121391 | 72121822 | 474_160949_6662(SOX9)_1 | 0 | — |
| chr17 | 72122718 | 72122972 | 474_160950_6662(SOX9)_2 | 0 | — |
| chr17 | 72123542 | 72124387 | 474_160951_6662(SOX9)_3 | 0 | — |
| chr18 | 47841826 | 47841950 | 474_158715_4087(SMAD2)_13a | 0 | — |
| chr18 | 47845339 | 47845484 | 474_158714_4087(SMAD2)_12a | 0 | — |
| chr18 | 47845662 | 47845800 | 474_158713_4087(SMAD2)_11a | 0 | — |
| chr18 | 47848474 | 47848687 | 474_158712_4087(SMAD2)_10a | 0 | — |
| chr18 | 47851273 | 47851327 | 474_158711_4087(SMAD2)_9a | 0 | — |
| chr18 | 47865058 | 47865133 | 474_158710_4087(SMAD2)_8a | 0 | — |
| chr18 | 47868322 | 47868457 | 474_158709_4087(SMAD2)_7a | 0 | — |
| chr18 | 47869242 | 47869436 | 474_158708_4087(SMAD2)_6a | 0 | — |
| chr18 | 47870474 | 47870564 | 474_158707_4087(SMAD2)_5a | 0 | — |
| chr18 | 47896520 | 47896803 | 474_158706_4087(SMAD2)_4a | 0 | — |
| chr18 | 51047046 | 51047295 | 474_158716_4089(SMAD4)_2 | 0 | — |
| chr18 | 51048685 | 51048860 | 474_158717_4089(SMAD4)_3 | 0 | — |
| chr18 | 51049294 | 51049324 | 474_158718_4089(SMAD4)_4 | 0 | — |
| chr18 | 51054780 | 51054993 | 474_158719_4089(SMAD4)_5 | 0 | — |
| chr18 | 51058124 | 51058244 | 474_158720_4089(SMAD4)_6 | 0 | — |
| chr18 | 51058339 | 51058456 | 474_158721_4089(SMAD4)_7 | 0 | — |
| chr18 | 51059865 | 51059916 | 474_158722_4089(SMAD4)_8 | 0 | — |
| chr18 | 51065422 | 51065606 | 474_158723_4089(SMAD4)_9 | 0 | — |
| chr18 | 51067018 | 51067187 | 474_158724_4089(SMAD4)_10 | 0 | — |
| chr18 | 51076637 | 51076776 | 474_158725_4089(SMAD4)_11 | 0 | — |
| chr18 | 51077306 | 51077419 | 474_158726_4089(SMAD4)_12 | 0 | — |
| chr18 | 51078255 | 51078467 | 474_158727_4089(SMAD4)_13 | 0 | — |
| chr18 | 52340787 | 52340878 | 431_46002_1630(DCC)_1 | 0 | — |
| chr18 | 52752053 | 52752374 | 431_46003_1630(DCC)_2 | 0 | — |
| chr18 | 52906043 | 52906328 | 431_46004_1630(DCC)_3 | 0 | — |
| chr18 | 52923706 | 52923857 | 431_46005_1630(DCC)_4 | 0 | — |
| chr18 | 52925233 | 52925370 | 431_46006_1630(DCC)_5 | 0 | — |
| chr18 | 53063304 | 53063459 | 431_46007_1630(DCC)_6 | 0 | — |
| chr18 | 53066045 | 53066166 | 431_46008_1630(DCC)_7 | 0 | — |
| chr18 | 53157355 | 53157512 | 431_46009_1630(DCC)_8 | 0 | — |
| chr18 | 53178961 | 53179116 | 431_46010_1630(DCC)_9 | 0 | — |
| chr18 | 53205215 | 53205364 | 431_46011_1630(DCC)_10 | 0 | — |
| chr18 | 53207678 | 53207817 | 431_46012_1630(DCC)_11 | 0 | — |
| chr18 | 53215547 | 53215597 | 431_46013_1630(DCC)_12 | 0 | — |
| chr18 | 53305577 | 53305719 | 431_46014_1630(DCC)_13 | 0 | — |
| chr18 | 53322046 | 53322157 | 431_46015_1630(DCC)_14 | 0 | — |
| chr18 | 53339712 | 53339907 | 431_46016_1630(DCC)_15 | 0 | — |
| chr18 | 53386042 | 53386138 | 431_46017_1630(DCC)_16 | 0 | — |
| chr18 | 53391654 | 53391887 | 431_46018_1630(DCC)_17 | 0 | — |
| chr18 | 53397307 | 53397446 | 431_46019_1630(DCC)_18 | 0 | — |
| chr18 | 53402785 | 53402893 | 431_46020_1630(DCC)_19 | 0 | — |
| chr18 | 53410451 | 53410646 | 431_46021_1630(DCC)_20 | 0 | — |
| chr18 | 53416123 | 53416156 | 431_46022_1630(DCC)_21 | 0 | — |
| chr18 | 53435143 | 53435209 | 431_46023_1630(DCC)_22 | 0 | — |
| chr18 | 53450499 | 53450662 | 431_46024_1630(DCC)_23 | 0 | — |
| chr18 | 53459231 | 53459458 | 431_46025_1630(DCC)_24 | 0 | — |
| chr18 | 53467893 | 53468010 | 431_46026_1630(DCC)_25 | 0 | — |
| chr18 | 53486796 | 53486958 | 431_46027_1630(DCC)_26 | 0 | — |
| chr18 | 53499297 | 53499510 | 431_46028_1630(DCC)_27 | 0 | — |
| chr18 | 53526616 | 53526759 | 431_46029_1630(DCC)_28 | 0 | — |
| chr18 | 53530563 | 53530653 | 431_46030_1630(DCC)_29 | 0 | — |
| chr19 | 1206913 | 1207203 | 475_170587_6794(STK11)_1a | 0 | — |
| chr19 | 1218416 | 1218500 | 475_170588_6794(STK11)_2a | 0 | — |
| chr19 | 1219323 | 1219413 | 475_170589_6794(STK11)_3a | 0 | — |
| chr19 | 1220372 | 1220505 | 475_170590_6794(STK11)_4a | 0 | — |
| chr19 | 1220580 | 1220717 | 475_170591_6794(STK11)_5a | 0 | — |
| chr19 | 1221212 | 1221340 | 475_170592_6794(STK11)_6a | 0 | — |
| chr19 | 1221948 | 1222006 | 475_170593_6794(STK11)_7a | 0 | — |
| chr19 | 1222984 | 1223172 | 475_170594_6794(STK11)_8a | 0 | — |
| chr19 | 1223637 | 1226446 | 475_170595_6794(STK11)_9a | 0 | — |
| chr19 | 1226453 | 1226663 | 475_170596_6794(STK11)_10a | 0 | — |
| chr19 | 1227592 | 1228082 | 475_170597_6794(STK11)_11a | 0 | — |
| chr19 | 2164184 | 2164265 | 431_55601_84444(DOT1L)_1 | 0 | — |
| chr19 | 2180712 | 2180756 | 431_55602_84444(DOT1L)_2 | 0 | — |
| chr19 | 2185854 | 2185929 | 431_55603_84444(DOT1L)_3 | 0 | — |
| chr19 | 2189731 | 2189795 | 431_55604_84444(DOT1L)_4 | 0 | — |
| chr19 | 2191011 | 2191240 | 431_55605_84444(DOT1L)_5 | 0 | — |
| chr19 | 2193688 | 2193783 | 431_55606_84444(DOT1L)_6 | 0 | — |
| chr19 | 2194514 | 2194577 | 431_55607_84444(DOT1L)_7 | 0 | — |
| chr19 | 2199883 | 2199939 | 431_55608_84444(DOT1L)_8 | 0 | — |
| chr19 | 2202699 | 2202779 | 431_55609_84444(DOT1L)_9 | 0 | — |
| chr19 | 2206728 | 2206797 | 431_55610_84444(DOT1L)_10 | 0 | — |
| chr19 | 2207573 | 2207680 | 431_55611_84444(DOT1L)_11 | 0 | — |
| chr19 | 2208934 | 2208976 | 431_55612_84444(DOT1L)_12 | 0 | — |
| chr19 | 2210399 | 2210510 | 431_55613_84444(DOT1L)_13 | 0 | — |
| chr19 | 2210620 | 2210855 | 431_55614_84444(DOT1L)_14 | 0 | — |
| chr19 | 2211098 | 2211212 | 431_55615_84444(DOT1L)_15 | 0 | — |
| chr19 | 2211750 | 2211842 | 431_55616_84444(DOT1L)_16 | 0 | — |
| chr19 | 2213538 | 2213640 | 431_55617_84444(DOT1L)_17 | 0 | — |
| chr19 | 2213848 | 2213986 | 431_55618_84444(DOT1L)_18 | 0 | — |
| chr19 | 2214470 | 2214596 | 431_55619_84444(DOT1L)_19 | 0 | — |
| chr19 | 2216280 | 2216765 | 431_55620_84444(DOT1L)_20 | 0 | — |
| chr19 | 2216954 | 2217090 | 431_55621_84444(DOT1L)_21 | 0 | — |
| chr19 | 2217771 | 2217918 | 431_55622_84444(DOT1L)_22 | 0 | — |
| chr19 | 2220107 | 2220222 | 431_55623_84444(DOT1L)_23 | 0 | — |
| chr19 | 2221975 | 2222559 | 431_55624_84444(DOT1L)_24 | 0 | — |
| chr19 | 2223280 | 2223486 | 431_55625_84444(DOT1L)_25 | 0 | — |
| chr19 | 2225387 | 2225452 | 431_55626_84444(DOT1L)_26 | 0 | — |
| chr19 | 2226182 | 2227127 | 431_55627_84444(DOT1L)_27 | 0 | — |
| chr19 | 2227736 | 2229792 | 431_55628_84444(DOT1L)_28 | 0 | — |
| chr19 | 3094651 | 3094787 | 462_70067_2767(GNA11)_1 | 0 | — |
| chr19 | 3110148 | 3110333 | 462_70068_2767(GNA11)_2 | 0 | — |
| chr19 | 3113329 | 3113484 | 462_70069_2767(GNA11)_3 | 0 | — |
| chr19 | 3114943 | 3115072 | 462_70070_2767(GNA11)_4 | 0 | — |
| chr19 | 3118923 | 3119053 | 462_70071_2767(GNA11)_5 | 0 | — |
| chr19 | 3119205 | 3119359 | 462_70072_2767(GNA11)_6 | 0 | — |
| chr19 | 3120988 | 3121179 | 462_70073_2767(GNA11)_7 | 0 | — |
| chr19 | 12938679 | 12938770 | 428_24220_811(CALR)_1 | 0 | — |
| chr19 | 12939133 | 12939235 | 428_24221_811(CALR)_2 | 0 | — |
| chr19 | 12939427 | 12939631 | 428_24222_811(CALR)_3 | 0 | — |
| chr19 | 12940052 | 12940147 | 428_24223_811(CALR)_4 | 0 | — |
| chr19 | 12940242 | 12940452 | 428_24224_811(CALR)_5 | 0 | — |
| chr19 | 12940540 | 12940654 | 428_24225_811(CALR)_6 | 0 | — |
| chr19 | 12940743 | 12940887 | 428_24226_811(CALR)_7 | 0 | — |
| chr19 | 12943536 | 12943629 | 428_24227_811(CALR)_8 | 0 | — |
| chr19 | 12943712 | 12943913 | 428_24228_811(CALR)_9 | 0 | — |
| chr19 | 17826742 | 17826910 | 463_81766_3718(JAK3)_24a | 0 | — |
| chr19 | 17830107 | 17830218 | 463_81765_3718(JAK3)_23a | 0 | — |
| chr19 | 17830502 | 17830620 | 463_81764_3718(JAK3)_22a | 0 | — |
| chr19 | 17831227 | 17831400 | 463_81763_3718(JAK3)_21a | 0 | — |
| chr19 | 17831673 | 17831798 | 463_81762_3718(JAK3)_20a | 0 | — |
| chr19 | 17832518 | 17832708 | 463_81761_3718(JAK3)_19a | 0 | — |
| chr19 | 17832789 | 17832929 | 463_81760_3718(JAK3)_18a | 0 | — |
| chr19 | 17834570 | 17834721 | 463_81759_3718(JAK3)_17a | 0 | — |
| chr19 | 17834851 | 17835003 | 463_81758_3718(JAK3)_16a | 0 | — |
| chr19 | 17835082 | 17835215 | 463_81757_3718(JAK3)_15a | 0 | — |
| chr19 | 17835923 | 17836051 | 463_81756_3718(JAK3)_14a | 0 | — |
| chr19 | 17837128 | 17837213 | 463_81755_3718(JAK3)_13a | 0 | — |
| chr19 | 17837931 | 17838063 | 463_81754_3718(JAK3)_12a | 0 | — |
| chr19 | 17838262 | 17838390 | 463_81753_3718(JAK3)_11a | 0 | — |
| chr19 | 17839476 | 17839663 | 463_81752_3718(JAK3)_10a | 0 | — |
| chr19 | 17840229 | 17840341 | 463_81751_3718(JAK3)_9a | 0 | — |
| chr19 | 17841388 | 17841546 | 463_81750_3718(JAK3)_8a | 0 | — |
| chr19 | 17841639 | 17841762 | 463_81749_3718(JAK3)_7a | 0 | — |
| chr19 | 17842315 | 17842610 | 463_81748_3718(JAK3)_6a | 0 | — |
| chr19 | 17843026 | 17843172 | 463_81747_3718(JAK3)_5a | 0 | — |
| chr19 | 17843379 | 17843491 | 463_81746_3718(JAK3)_4a | 0 | — |
| chr19 | 17843776 | 17843900 | 463_81745_3718(JAK3)_3a | 0 | — |
| chr19 | 17844233 | 17844430 | 463_81744_3718(JAK3)_2a | 0 | — |
| chr19 | 17847945 | 17848061 | 463_81743_3718(JAK3)_1a | 0 | — |
| chr19 | 50398851 | 50399053 | 470_129016_5424(POLD1)_2 | 0 | — |
| chr19 | 50399370 | 50399484 | 470_129017_5424(POLD1)_3 | 0 | — |
| chr19 | 50401777 | 50401924 | 470_129018_5424(POLD1)_4 | 0 | — |
| chr19 | 50401998 | 50402124 | 470_129019_5424(POLD1)_5 | 0 | — |
| chr19 | 50402204 | 50402373 | 470_129020_5424(POLD1)_6 | 0 | — |
| chr19 | 50402453 | 50402535 | 470_129021_5424(POLD1)_7 | 0 | — |
| chr19 | 50402611 | 50402741 | 470_129022_5424(POLD1)_8 | 0 | — |
| chr19 | 50403052 | 50403219 | 470_129023_5424(POLD1)_9 | 0 | — |
| chr19 | 50403492 | 50403597 | 470_129024_5424(POLD1)_10 | 0 | — |
| chr19 | 50406181 | 50406322 | 470_129025_5424(POLD1)_11 | 0 | — |
| chr19 | 50406406 | 50406517 | 470_129026_5424(POLD1)_12 | 0 | — |
| chr19 | 50406982 | 50407174 | 470_129027_5424(POLD1)_13 | 0 | — |
| chr19 | 50407326 | 50407415 | 470_129028_5424(POLD1)_14 | 0 | — |
| chr19 | 50408706 | 50408901 | 470_129029_5424(POLD1)_15 | 0 | — |
| chr19 | 50409121 | 50409235 | 470_129030_5424(POLD1)_16 | 0 | — |
| chr19 | 50409518 | 50409666 | 470_129031_5424(POLD1)_17 | 0 | — |
| chr19 | 50413425 | 50413521 | 470_129032_5424(POLD1)_18 | 0 | — |
| chr19 | 50413741 | 50413879 | 470_129033_5424(POLD1)_19 | 0 | — |
| chr19 | 50414814 | 50414990 | 470_129034_5424(POLD1)_20 | 0 | — |
| chr19 | 50415437 | 50415590 | 470_129035_5424(POLD1)_21 | 0 | — |
| chr19 | 50415723 | 50415826 | 470_129036_5424(POLD1)_22 | 0 | — |
| chr19 | 50416395 | 50416528 | 470_129037_5424(POLD1)_23 | 0 | — |
| chr19 | 50416609 | 50416723 | 470_129038_5424(POLD1)_24 | 0 | — |
| chr19 | 50417044 | 50417097 | 470_129039_5424(POLD1)_25 | 0 | — |
| chr19 | 50417171 | 50417269 | 470_129040_5424(POLD1)_26 | 0 | — |
| chr19 | 50417841 | 50417947 | 470_129041_5424(POLD1)_27 | 0 | — |
| chr2 | 29193223 | 29193922 | 426_1998_238(ALK)_29 | 0 | — |
| chr2 | 29196769 | 29196860 | 426_1997_238(ALK)_28 | 0 | — |
| chr2 | 29197541 | 29197676 | 426_1996_238(ALK)_27 | 0 | — |
| chr2 | 29207170 | 29207272 | 426_1995_238(ALK)_26 | 0 | — |
| chr2 | 29209785 | 29209878 | 426_1994_238(ALK)_25 | 0 | — |
| chr2 | 29213983 | 29214081 | 426_1993_238(ALK)_24 | 0 | — |
| chr2 | 29220705 | 29220835 | 426_1992_238(ALK)_23 | 0 | — |
| chr2 | 29222343 | 29222408 | 426_1991_238(ALK)_22 | 0 | — |
| chr2 | 29222516 | 29222607 | 426_1990_238(ALK)_21 | 0 | — |
| chr2 | 29223341 | 29223528 | 426_1989_238(ALK)_20 | 0 | — |
| chr2 | 29225460 | 29225565 | 426_1988_238(ALK)_19 | 0 | — |
| chr2 | 29226921 | 29227074 | 426_1987_238(ALK)_18 | 0 | — |
| chr2 | 29227573 | 29227672 | 426_1986_238(ALK)_17 | 0 | — |
| chr2 | 29228883 | 29229066 | 426_1985_238(ALK)_16 | 0 | — |
| chr2 | 29232303 | 29232448 | 426_1984_238(ALK)_15 | 0 | — |
| chr2 | 29233564 | 29233696 | 426_1983_238(ALK)_14 | 0 | — |
| chr2 | 29239679 | 29239830 | 426_1982_238(ALK)_13 | 0 | — |
| chr2 | 29251104 | 29251267 | 426_1981_238(ALK)_12 | 0 | — |
| chr2 | 29275098 | 29275227 | 426_1980_238(ALK)_11 | 0 | — |
| chr2 | 29275401 | 29275496 | 426_1979_238(ALK)_10 | 0 | — |
| chr2 | 29296887 | 29297057 | 426_1978_238(ALK)_9 | 0 | — |
| chr2 | 29318303 | 29318404 | 426_1977_238(ALK)_8 | 0 | — |
| chr2 | 29320750 | 29320882 | 426_1976_238(ALK)_7 | 0 | — |
| chr2 | 29328349 | 29328481 | 426_1975_238(ALK)_6 | 0 | — |
| chr2 | 29383731 | 29383859 | 426_1974_238(ALK)_5 | 0 | — |
| chr2 | 29531914 | 29532116 | 426_1973_238(ALK)_4 | 0 | — |
| chr2 | 29694849 | 29695014 | 426_1972_238(ALK)_3 | 0 | — |
| chr2 | 29717577 | 29717697 | 426_1971_238(ALK)_2 | 0 | — |
| chr2 | 29919992 | 29920659 | 426_1970_238(ALK)_1 | 0 | — |
| chr2 | 43224318 | 43225752 | 477_180793_678(ZFP36L2)_2 | 0 | — |
| chr2 | 43226264 | 43226315 | 477_180792_678(ZFP36L2)_1 | 0 | — |
| chr2 | 147845152 | 147845207 | 426_921_92(ACVR2A)_2 | 0 | — |
| chr2 | 147896300 | 147896508 | 426_922_92(ACVR2A)_3 | 0 | — |
| chr2 | 147899457 | 147899567 | 426_923_92(ACVR2A)_4 | 0 | — |
| chr2 | 147899743 | 147899898 | 426_924_92(ACVR2A)_5 | 0 | — |
| chr2 | 147915190 | 147915334 | 426_925_92(ACVR2A)_6 | 0 | — |
| chr2 | 147917282 | 147917426 | 426_926_92(ACVR2A)_7 | 0 | — |
| chr2 | 147918446 | 147918592 | 426_927_92(ACVR2A)_8 | 0 | — |
| chr2 | 147920229 | 147920344 | 426_928_92(ACVR2A)_9 | 0 | — |
| chr2 | 147922972 | 147923111 | 426_929_92(ACVR2A)_10 | 0 | — |
| chr2 | 147926030 | 147926161 | 426_930_92(ACVR2A)_11 | 0 | — |
| chr2 | 147927079 | 147927274 | 426_931_92(ACVR2A)_12 | 0 | — |
| chr2 | 208237078 | 208237169 | 463_79499_3417(IDH1)_13a | 0 | — |
| chr2 | 208239070 | 208239233 | 463_79498_3417(IDH1)_12a | 0 | — |
| chr2 | 208239862 | 208240003 | 463_79497_3417(IDH1)_11a | 0 | — |
| chr2 | 208241993 | 208242145 | 463_79496_3417(IDH1)_10a | 0 | — |
| chr2 | 208243426 | 208243604 | 463_79495_3417(IDH1)_9a | 0 | — |
| chr2 | 208245318 | 208245424 | 463_79494_3417(IDH1)_8a | 0 | — |
| chr2 | 208248368 | 208248660 | 463_79493_3417(IDH1)_7a | 0 | — |
| chr2 | 208251429 | 208251551 | 463_79492_3417(IDH1)_6a | 0 | — |
| chr2 | 211383614 | 211384060 | 461_58800_2066(ERBB4)_29a | 0 | — |
| chr2 | 211386852 | 211387150 | 461_58799_2066(ERBB4)_28a | 0 | — |
| chr2 | 211387944 | 211387992 | 461_58798_2066(ERBB4)_27a | 0 | — |
| chr2 | 211420440 | 211420611 | 461_58797_2066(ERBB4)_26a | 0 | — |
| chr2 | 211422006 | 211422104 | 461_58796_2066(ERBB4)_25a | 0 | — |
| chr2 | 211424154 | 211424301 | 461_58795_2066(ERBB4)_24a | 0 | — |
| chr2 | 211428407 | 211428483 | 461_58794_2066(ERBB4)_23a | 0 | — |
| chr2 | 211430944 | 211431100 | 461_58793_2066(ERBB4)_22a | 0 | — |
| chr2 | 211561902 | 211562088 | 461_58792_2066(ERBB4)_21a | 0 | — |
| chr2 | 211619176 | 211619275 | 461_58791_2066(ERBB4)_20a | 0 | — |
| chr2 | 211623921 | 211624044 | 461_58790_2066(ERBB4)_19a | 0 | — |
| chr2 | 211630461 | 211630594 | 461_58789_2066(ERBB4)_18a | 0 | — |
| chr2 | 211657753 | 211657828 | 461_58788_2066(ERBB4)_17a | 0 | — |
| chr2 | 211657949 | 211657994 | 461_58787_2066(ERBB4)_16a | 0 | — |
| chr2 | 211665322 | 211665477 | 461_58786_2066(ERBB4)_15a | 0 | — |
| chr2 | 211673163 | 211673257 | 461_58785_2066(ERBB4)_14a | 0 | — |
| chr2 | 211679051 | 211679184 | 461_58784_2066(ERBB4)_13a | 0 | — |
| chr2 | 211701966 | 211702166 | 461_58783_2066(ERBB4)_12a | 0 | — |
| chr2 | 211704103 | 211704194 | 461_58782_2066(ERBB4)_11a | 0 | — |
| chr2 | 211705317 | 211705391 | 461_58781_2066(ERBB4)_10a | 0 | — |
| chr2 | 211712049 | 211712176 | 461_58780_2066(ERBB4)_9a | 0 | — |
| chr2 | 211713534 | 211713648 | 461_58779_2066(ERBB4)_8a | 0 | — |
| chr2 | 211722392 | 211722534 | 461_58778_2066(ERBB4)_7a | 0 | — |
| chr2 | 211725075 | 211725194 | 461_58777_2066(ERBB4)_6a | 0 | — |
| chr2 | 211750638 | 211750704 | 461_58776_2066(ERBB4)_5a | 0 | — |
| chr2 | 211788024 | 211788159 | 461_58775_2066(ERBB4)_4a | 0 | — |
| chr2 | 211947429 | 211947616 | 461_58774_2066(ERBB4)_3a | 0 | — |
| chr2 | 212124751 | 212124903 | 461_58773_2066(ERBB4)_2a | 0 | — |
| chr2 | 212538448 | 212538530 | 461_58772_2066(ERBB4)_1a | 0 | — |
| chr20 | 37384153 | 37384403 | 475_169917_6714(SRC)_5a | 0 | — |
| chr20 | 37386074 | 37386174 | 475_169918_6714(SRC)_6a | 0 | — |
| chr20 | 37393894 | 37393993 | 475_169919_6714(SRC)_7a | 0 | — |
| chr20 | 37394173 | 37394277 | 475_169920_6714(SRC)_8a | 0 | — |
| chr20 | 37396161 | 37396311 | 475_169921_6714(SRC)_9a | 0 | — |
| chr20 | 37397698 | 37397854 | 475_169922_6714(SRC)_10a | 0 | — |
| chr20 | 37400114 | 37400294 | 475_169923_6714(SRC)_11a | 0 | — |
| chr20 | 37401601 | 37401678 | 475_169924_6714(SRC)_12a | 0 | — |
| chr20 | 37402434 | 37402588 | 475_169925_6714(SRC)_13a | 0 | — |
| chr20 | 37402748 | 37402880 | 475_169926_6714(SRC)_14a | 0 | — |
| chr20 | 37403170 | 37403379 | 475_169927_6714(SRC)_15a | 0 | — |
| chr20 | 56370157 | 56370340 | 427_16080_6790(AURKA)_11a | 0 | — |
| chr20 | 56370484 | 56370659 | 427_16079_6790(AURKA)_10a | 0 | — |
| chr20 | 56373407 | 56373556 | 427_16078_6790(AURKA)_9a | 0 | — |
| chr20 | 56381432 | 56381571 | 427_16077_6790(AURKA)_8a | 0 | — |
| chr20 | 56382984 | 56383176 | 427_16076_6790(AURKA)_7a | 0 | — |
| chr20 | 56384269 | 56384324 | 427_16075_6790(AURKA)_6a | 0 | — |
| chr20 | 56386256 | 56386533 | 427_16074_6790(AURKA)_5a | 0 | — |
| chr20 | 56388155 | 56388197 | 427_16073_6790(AURKA)_4a | 0 | — |
| chr20 | 58840106 | 58840886 | 462_70269_2778(GNAS)_1 | 0 | — |
| chr20 | 58841342 | 58841875 | 462_70270_2778(GNAS)_2 | 0 | — |
| chr20 | 58852980 | 58855333 | 462_70271_2778(GNAS)_3 | 0 | — |
| chr20 | 58889124 | 58889353 | 462_70272_2778(GNAS)_4 | 0 | — |
| chr20 | 58891370 | 58891865 | 462_70273_2778(GNAS)_5 | 0 | — |
| chr20 | 58892109 | 58892209 | 462_70274_2778(GNAS)_6 | 0 | — |
| chr20 | 58895611 | 58895684 | 462_70275_2778(GNAS)_7 | 0 | — |
| chr20 | 58898940 | 58898985 | 462_70276_2778(GNAS)_8 | 0 | — |
| chr20 | 58903527 | 58903585 | 462_70277_2778(GNAS)_9 | 0 | — |
| chr20 | 58903671 | 58903791 | 462_70278_2778(GNAS)_10 | 0 | — |
| chr20 | 58905382 | 58905480 | 462_70279_2778(GNAS)_11 | 0 | — |
| chr20 | 58909161 | 58909216 | 462_70280_2778(GNAS)_12 | 0 | — |
| chr20 | 58909349 | 58909423 | 462_70281_2778(GNAS)_13 | 0 | — |
| chr20 | 58909520 | 58909579 | 462_70282_2778(GNAS)_14 | 0 | — |
| chr20 | 58909683 | 58909804 | 462_70283_2778(GNAS)_15 | 0 | — |
| chr20 | 58909950 | 58910081 | 462_70284_2778(GNAS)_16 | 0 | — |
| chr20 | 58910333 | 58910401 | 462_70285_2778(GNAS)_17 | 0 | — |
| chr20 | 58910682 | 58910829 | 462_70286_2778(GNAS)_18 | 0 | — |
| chr22 | 23787169 | 23787262 | 474_160328_6598(SMARCB1)_1 | 0 | — |
| chr22 | 23791755 | 23791894 | 474_160329_6598(SMARCB1)_2 | 0 | — |
| chr22 | 23793558 | 23793688 | 474_160330_6598(SMARCB1)_3 | 0 | — |
| chr22 | 23800943 | 23801135 | 474_160331_6598(SMARCB1)_4 | 0 | — |
| chr22 | 23803294 | 23803422 | 474_160332_6598(SMARCB1)_5 | 0 | — |
| chr22 | 23816769 | 23816936 | 474_160333_6598(SMARCB1)_6 | 0 | — |
| chr22 | 23825224 | 23825415 | 474_160334_6598(SMARCB1)_7 | 0 | — |
| chr22 | 23833571 | 23833703 | 474_160335_6598(SMARCB1)_8 | 0 | — |
| chr22 | 23834140 | 23834180 | 474_160336_6598(SMARCB1)_9 | 0 | — |
| chr22 | 28883766 | 28884066 | 478_203540_84133(ZNRF3)_1 | 0 | — |
| chr22 | 28917388 | 28917481 | 478_203541_84133(ZNRF3)_2 | 0 | — |
| chr22 | 28986622 | 28986639 | 478_203542_84133(ZNRF3)_3 | 0 | — |
| chr22 | 28987075 | 28987201 | 478_203543_84133(ZNRF3)_4 | 0 | — |
| chr22 | 29031584 | 29031631 | 478_203544_84133(ZNRF3)_5 | 0 | — |
| chr22 | 29042494 | 29042569 | 478_203545_84133(ZNRF3)_6 | 0 | — |
| chr22 | 29043298 | 29043430 | 478_203546_84133(ZNRF3)_7 | 0 | — |
| chr22 | 29044779 | 29044890 | 478_203547_84133(ZNRF3)_8 | 0 | — |
| chr22 | 29046715 | 29046883 | 478_203548_84133(ZNRF3)_9 | 0 | — |
| chr22 | 29048388 | 29048491 | 478_203549_84133(ZNRF3)_10 | 0 | — |
| chr22 | 29049196 | 29050948 | 478_203550_84133(ZNRF3)_11 | 0 | — |
| chr22 | 29053578 | 29053622 | 478_203551_84133(ZNRF3)_12 | 0 | — |
| chr22 | 41093004 | 41093098 | 461_58342_2033(EP300)_1a | 0 | — |
| chr22 | 41117186 | 41117821 | 461_58343_2033(EP300)_2a | 0 | — |
| chr22 | 41125863 | 41126040 | 461_58344_2033(EP300)_3a | 0 | — |
| chr22 | 41127486 | 41127748 | 461_58345_2033(EP300)_4a | 0 | — |
| chr22 | 41129889 | 41130003 | 461_58346_2033(EP300)_5a | 0 | — |
| chr22 | 41131387 | 41131633 | 461_58347_2033(EP300)_6a | 0 | — |
| chr22 | 41135812 | 41135906 | 461_58348_2033(EP300)_7a | 0 | — |
| chr22 | 41137652 | 41137790 | 461_58349_2033(EP300)_8a | 0 | — |
| chr22 | 41140139 | 41140257 | 461_58350_2033(EP300)_9a | 0 | — |
| chr22 | 41141047 | 41141222 | 461_58351_2033(EP300)_10a | 0 | — |
| chr22 | 41146738 | 41146816 | 461_58352_2033(EP300)_11a | 0 | — |
| chr22 | 41147836 | 41147946 | 461_58353_2033(EP300)_12a | 0 | — |
| chr22 | 41149037 | 41149175 | 461_58354_2033(EP300)_13a | 0 | — |
| chr22 | 41149760 | 41150198 | 461_58355_2033(EP300)_14a | 0 | — |
| chr22 | 41151832 | 41152012 | 461_58356_2033(EP300)_15a | 0 | — |
| chr22 | 41152205 | 41152350 | 461_58357_2033(EP300)_16a | 0 | — |
| chr22 | 41154994 | 41155113 | 461_58358_2033(EP300)_17a | 0 | — |
| chr22 | 41157168 | 41157408 | 461_58359_2033(EP300)_18a | 0 | — |
| chr22 | 41158411 | 41158500 | 461_58360_2033(EP300)_19a | 0 | — |
| chr22 | 41160641 | 41160722 | 461_58361_2033(EP300)_20a | 0 | — |
| chr22 | 41162722 | 41162779 | 461_58362_2033(EP300)_21a | 0 | — |
| chr22 | 41164052 | 41164130 | 461_58363_2033(EP300)_22a | 0 | — |
| chr22 | 41166598 | 41166666 | 461_58364_2033(EP300)_23a | 0 | — |
| chr22 | 41168448 | 41168599 | 461_58365_2033(EP300)_24a | 0 | — |
| chr22 | 41168720 | 41168867 | 461_58366_2033(EP300)_25a | 0 | — |
| chr22 | 41169502 | 41169616 | 461_58367_2033(EP300)_26a | 0 | — |
| chr22 | 41170405 | 41170571 | 461_58368_2033(EP300)_27a | 0 | — |
| chr22 | 41172498 | 41172663 | 461_58369_2033(EP300)_28a | 0 | — |
| chr22 | 41173622 | 41173784 | 461_58370_2033(EP300)_29a | 0 | — |
| chr22 | 41176246 | 41176528 | 461_58371_2033(EP300)_30a | 0 | — |
| chr22 | 41176772 | 41178956 | 461_58372_2033(EP300)_31a | 0 | — |
| chr3 | 10141847 | 10142187 | 477_191808_7428(VHL)_1 | 0 | — |
| chr3 | 10146513 | 10146636 | 477_191809_7428(VHL)_2 | 0 | — |
| chr3 | 10149786 | 10150367 | 477_191810_7428(VHL)_3 | 0 | — |
| chr3 | 36993547 | 36993663 | 466_106456_4292(MLH1)_1 | 0 | — |
| chr3 | 36993768 | 36993890 | 466_106457_4292(MLH1)_2 | 0 | — |
| chr3 | 36996618 | 36996709 | 466_106458_4292(MLH1)_3 | 0 | — |
| chr3 | 36997155 | 36997300 | 466_106459_4292(MLH1)_4 | 0 | — |
| chr3 | 37000954 | 37001053 | 466_106460_4292(MLH1)_5 | 0 | — |
| chr3 | 37004400 | 37004474 | 466_106461_4292(MLH1)_6 | 0 | — |
| chr3 | 37006990 | 37007063 | 466_106462_4292(MLH1)_7 | 0 | — |
| chr3 | 37008813 | 37008905 | 466_106463_4292(MLH1)_8 | 0 | — |
| chr3 | 37011819 | 37011862 | 466_106464_4292(MLH1)_9 | 0 | — |
| chr3 | 37012010 | 37012099 | 466_106465_4292(MLH1)_10 | 0 | — |
| chr3 | 37014431 | 37014544 | 466_106466_4292(MLH1)_11 | 0 | — |
| chr3 | 37017505 | 37017599 | 466_106467_4292(MLH1)_12 | 0 | — |
| chr3 | 37020309 | 37020463 | 466_106468_4292(MLH1)_13 | 0 | — |
| chr3 | 37025636 | 37026007 | 466_106469_4292(MLH1)_14 | 0 | — |
| chr3 | 37028783 | 37028932 | 466_106470_4292(MLH1)_15 | 0 | — |
| chr3 | 37040185 | 37040294 | 466_106471_4292(MLH1)_16 | 0 | — |
| chr3 | 37042267 | 37042331 | 466_106472_4292(MLH1)_17 | 0 | — |
| chr3 | 37047518 | 37047683 | 466_106473_4292(MLH1)_18 | 0 | — |
| chr3 | 37048516 | 37048609 | 466_106474_4292(MLH1)_19 | 0 | — |
| chr3 | 37048903 | 37049017 | 466_106475_4292(MLH1)_20 | 0 | — |
| chr3 | 37050485 | 37050653 | 466_106476_4292(MLH1)_21 | 0 | — |
| chr3 | 41224068 | 41224081 | 429_36856_1499(CTNNB1)_7 | 0 | — |
| chr3 | 41224525 | 41224753 | 429_36857_1499(CTNNB1)_8 | 0 | — |
| chr3 | 41224953 | 41225207 | 429_36858_1499(CTNNB1)_9 | 0 | — |
| chr3 | 41225333 | 41225572 | 429_36859_1499(CTNNB1)_10 | 0 | — |
| chr3 | 41225659 | 41225861 | 429_36860_1499(CTNNB1)_11 | 0 | — |
| chr3 | 41227207 | 41227352 | 429_36861_1499(CTNNB1)_12 | 0 | — |
| chr3 | 41233340 | 41233444 | 429_36862_1499(CTNNB1)_13 | 0 | — |
| chr3 | 41233528 | 41233867 | 429_36863_1499(CTNNB1)_14 | 0 | — |
| chr3 | 41234138 | 41234297 | 429_36864_1499(CTNNB1)_15 | 0 | — |
| chr3 | 41235723 | 41235843 | 429_36865_1499(CTNNB1)_16 | 0 | — |
| chr3 | 41236348 | 41236499 | 429_36866_1499(CTNNB1)_17 | 0 | — |
| chr3 | 41236587 | 41236709 | 429_36867_1499(CTNNB1)_18 | 0 | — |
| chr3 | 41238015 | 41238076 | 429_36868_1499(CTNNB1)_19 | 0 | — |
| chr3 | 41239133 | 41239342 | 429_36869_1499(CTNNB1)_20 | 0 | — |
| chr3 | 142449428 | 142449602 | 427_14899_545(ATR)_47 | 0 | — |
| chr3 | 142450465 | 142453233 | 427_14898_545(ATR)_46 | 0 | — |
| chr3 | 142457603 | 142457755 | 427_14897_545(ATR)_45 | 0 | — |
| chr3 | 142458957 | 142459111 | 427_14896_545(ATR)_44 | 0 | — |
| chr3 | 142459226 | 142459383 | 427_14895_545(ATR)_43 | 0 | — |
| chr3 | 142461939 | 142462090 | 427_14894_545(ATR)_42 | 0 | — |
| chr3 | 142465096 | 142465240 | 427_14893_545(ATR)_41 | 0 | — |
| chr3 | 142466323 | 142466533 | 427_14892_545(ATR)_40 | 0 | — |
| chr3 | 142467933 | 142468068 | 427_14891_545(ATR)_39 | 0 | — |
| chr3 | 142469336 | 142469569 | 427_14890_545(ATR)_38 | 0 | — |
| chr3 | 142470085 | 142470183 | 427_14889_545(ATR)_37 | 0 | — |
| chr3 | 142485139 | 142485282 | 427_14888_545(ATR)_36 | 0 | — |
| chr3 | 142493131 | 142493311 | 427_14887_545(ATR)_35 | 0 | — |
| chr3 | 142496360 | 142496520 | 427_14886_545(ATR)_34 | 0 | — |
| chr3 | 142497012 | 142497192 | 427_14885_545(ATR)_33 | 0 | — |
| chr3 | 142498596 | 142498774 | 427_14884_545(ATR)_32 | 0 | — |
| chr3 | 142499626 | 142499718 | 427_14883_545(ATR)_31 | 0 | — |
| chr3 | 142503361 | 142503453 | 427_14882_545(ATR)_30 | 0 | — |
| chr3 | 142505138 | 142505303 | 427_14881_545(ATR)_29 | 0 | — |
| chr3 | 142507930 | 142508109 | 427_14880_545(ATR)_28 | 0 | — |
| chr3 | 142512259 | 142512470 | 427_14879_545(ATR)_27 | 0 | — |
| chr3 | 142513500 | 142513638 | 427_14878_545(ATR)_26 | 0 | — |
| chr3 | 142515394 | 142515515 | 427_14877_545(ATR)_25 | 0 | — |
| chr3 | 142519668 | 142519784 | 427_14876_545(ATR)_24 | 0 | — |
| chr3 | 142522727 | 142522841 | 427_14875_545(ATR)_23 | 0 | — |
| chr3 | 142523992 | 142524199 | 427_14874_545(ATR)_22 | 0 | — |
| chr3 | 142535079 | 142535205 | 427_14873_545(ATR)_21 | 0 | — |
| chr3 | 142536107 | 142536201 | 427_14872_545(ATR)_20 | 0 | — |
| chr3 | 142538481 | 142538625 | 427_14871_545(ATR)_19 | 0 | — |
| chr3 | 142540903 | 142541034 | 427_14870_545(ATR)_18 | 0 | — |
| chr3 | 142542664 | 142542757 | 427_14869_545(ATR)_17 | 0 | — |
| chr3 | 142547724 | 142547910 | 427_14868_545(ATR)_16 | 0 | — |
| chr3 | 142549478 | 142549673 | 427_14867_545(ATR)_15 | 0 | — |
| chr3 | 142550131 | 142550302 | 427_14866_545(ATR)_14 | 0 | — |
| chr3 | 142553226 | 142553398 | 427_14865_545(ATR)_13 | 0 | — |
| chr3 | 142553639 | 142553740 | 427_14864_545(ATR)_12 | 0 | — |
| chr3 | 142553824 | 142554015 | 427_14863_545(ATR)_11 | 0 | — |
| chr3 | 142555876 | 142556139 | 427_14862_545(ATR)_10 | 0 | — |
| chr3 | 142556382 | 142556575 | 427_14861_545(ATR)_9 | 0 | — |
| chr3 | 142558623 | 142558776 | 427_14860_545(ATR)_8 | 0 | — |
| chr3 | 142559250 | 142559441 | 427_14859_545(ATR)_7 | 0 | — |
| chr3 | 142560262 | 142560454 | 427_14858_545(ATR)_6 | 0 | — |
| chr3 | 142561242 | 142561421 | 427_14857_545(ATR)_5 | 0 | — |
| chr3 | 142562231 | 142563109 | 427_14856_545(ATR)_4 | 0 | — |
| chr3 | 142566120 | 142566261 | 427_14855_545(ATR)_3 | 0 | — |
| chr3 | 142568062 | 142568154 | 427_14854_545(ATR)_2 | 0 | — |
| chr3 | 142578645 | 142578704 | 427_14853_545(ATR)_1 | 0 | — |
| chr3 | 179198825 | 179199177 | 470_127804_5290(PIK3CA)_2 | 0 | — |
| chr3 | 179199689 | 179199899 | 470_127805_5290(PIK3CA)_3 | 0 | — |
| chr3 | 179201289 | 179201540 | 470_127806_5290(PIK3CA)_4 | 0 | — |
| chr3 | 179203543 | 179203789 | 470_127807_5290(PIK3CA)_5 | 0 | — |
| chr3 | 179204502 | 179204588 | 470_127808_5290(PIK3CA)_6 | 0 | — |
| chr3 | 179209594 | 179209700 | 470_127809_5290(PIK3CA)_7 | 0 | — |
| chr3 | 179210185 | 179210338 | 470_127810_5290(PIK3CA)_8 | 0 | — |
| chr3 | 179210430 | 179210565 | 470_127811_5290(PIK3CA)_9 | 0 | — |
| chr3 | 179218209 | 179218334 | 470_127812_5290(PIK3CA)_10 | 0 | — |
| chr3 | 179219195 | 179219277 | 470_127813_5290(PIK3CA)_11 | 0 | — |
| chr3 | 179219570 | 179219735 | 470_127814_5290(PIK3CA)_12 | 0 | — |
| chr3 | 179219948 | 179220052 | 470_127815_5290(PIK3CA)_13 | 0 | — |
| chr3 | 179220985 | 179221157 | 470_127816_5290(PIK3CA)_14 | 0 | — |
| chr3 | 179224080 | 179224187 | 470_127817_5290(PIK3CA)_15 | 0 | — |
| chr3 | 179224699 | 179224821 | 470_127818_5290(PIK3CA)_16 | 0 | — |
| chr3 | 179225961 | 179226040 | 470_127819_5290(PIK3CA)_17 | 0 | — |
| chr3 | 179229271 | 179229442 | 470_127820_5290(PIK3CA)_18 | 0 | — |
| chr3 | 179230003 | 179230121 | 470_127821_5290(PIK3CA)_19 | 0 | — |
| chr3 | 179230224 | 179230376 | 470_127822_5290(PIK3CA)_20 | 0 | — |
| chr3 | 179234093 | 179234364 | 470_127823_5290(PIK3CA)_21 | 0 | — |
| chr4 | 1793934 | 1794043 | 461_60335_2261(FGFR3)_2a | 0 | — |
| chr4 | 1799253 | 1799523 | 461_60336_2261(FGFR3)_3a | 0 | — |
| chr4 | 1799746 | 1799812 | 461_60337_2261(FGFR3)_4a | 0 | — |
| chr4 | 1801366 | 1801536 | 461_60338_2261(FGFR3)_5a | 0 | — |
| chr4 | 1801619 | 1801743 | 461_60339_2261(FGFR3)_6a | 0 | — |
| chr4 | 1801834 | 1802025 | 461_60340_2261(FGFR3)_7a | 0 | — |
| chr4 | 1802913 | 1803064 | 461_60341_2261(FGFR3)_8a | 0 | — |
| chr4 | 1803691 | 1803836 | 461_60342_2261(FGFR3)_9a | 0 | — |
| chr4 | 1804329 | 1804520 | 461_60343_2261(FGFR3)_10a | 0 | — |
| chr4 | 1804823 | 1804969 | 461_60344_2261(FGFR3)_11a | 0 | — |
| chr4 | 1805354 | 1805476 | 461_60345_2261(FGFR3)_12a | 0 | — |
| chr4 | 1805558 | 1805669 | 461_60346_2261(FGFR3)_13a | 0 | — |
| chr4 | 1805749 | 1805940 | 461_60347_2261(FGFR3)_14a | 0 | — |
| chr4 | 1806050 | 1806173 | 461_60348_2261(FGFR3)_15a | 0 | — |
| chr4 | 1806256 | 1806327 | 461_60349_2261(FGFR3)_16a | 0 | — |
| chr4 | 1806545 | 1806683 | 461_60350_2261(FGFR3)_17a | 0 | — |
| chr4 | 1806828 | 1806934 | 461_60351_2261(FGFR3)_18a | 0 | — |
| chr4 | 1807115 | 1807262 | 461_60352_2261(FGFR3)_19a | 0 | — |
| chr4 | 54258768 | 54258817 | 470_126838_5156(PDGFRA)_3a | 0 | — |
| chr4 | 54261094 | 54261412 | 470_126839_5156(PDGFRA)_4a | 0 | — |
| chr4 | 54263666 | 54263927 | 470_126840_5156(PDGFRA)_5a | 0 | — |
| chr4 | 54264918 | 54265049 | 470_126841_5156(PDGFRA)_6a | 0 | — |
| chr4 | 54267288 | 54267460 | 470_126842_5156(PDGFRA)_7a | 0 | — |
| chr4 | 54267551 | 54267741 | 470_126843_5156(PDGFRA)_8a | 0 | — |
| chr4 | 54270632 | 54270748 | 470_126844_5156(PDGFRA)_9a | 0 | — |
| chr4 | 54272393 | 54272520 | 470_126845_5156(PDGFRA)_10a | 0 | — |
| chr4 | 54273536 | 54273730 | 470_126846_5156(PDGFRA)_11a | 0 | — |
| chr4 | 54274530 | 54274625 | 470_126847_5156(PDGFRA)_12a | 0 | — |
| chr4 | 54274840 | 54274973 | 470_126848_5156(PDGFRA)_13a | 0 | — |
| chr4 | 54277387 | 54277492 | 470_126849_5156(PDGFRA)_14a | 0 | — |
| chr4 | 54277895 | 54278006 | 470_126850_5156(PDGFRA)_15a | 0 | — |
| chr4 | 54278361 | 54278515 | 470_126851_5156(PDGFRA)_16a | 0 | — |
| chr4 | 54280315 | 54280482 | 470_126852_5156(PDGFRA)_17a | 0 | — |
| chr4 | 54281580 | 54281978 | 470_126853_5156(PDGFRA)_18a | 0 | — |
| chr4 | 54285370 | 54285486 | 470_126854_5156(PDGFRA)_19a | 0 | — |
| chr4 | 54285840 | 54285963 | 470_126855_5156(PDGFRA)_20a | 0 | — |
| chr4 | 54287429 | 54287541 | 470_126856_5156(PDGFRA)_21a | 0 | — |
| chr4 | 54288798 | 54288898 | 470_126857_5156(PDGFRA)_22a | 0 | — |
| chr4 | 54289008 | 54289114 | 470_126858_5156(PDGFRA)_23a | 0 | — |
| chr4 | 54290312 | 54290554 | 470_126859_5156(PDGFRA)_24a | 0 | — |
| chr4 | 54295124 | 54295272 | 470_126860_5156(PDGFRA)_25a | 0 | — |
| chr4 | 54658014 | 54658081 | 464_88966_3815(KIT)_1 | 0 | — |
| chr4 | 54695511 | 54695781 | 464_88967_3815(KIT)_2 | 0 | — |
| chr4 | 54698283 | 54698565 | 464_88968_3815(KIT)_3 | 0 | — |
| chr4 | 54699629 | 54699766 | 464_88969_3815(KIT)_4 | 0 | — |
| chr4 | 54703720 | 54703892 | 464_88970_3815(KIT)_5 | 0 | — |
| chr4 | 54707097 | 54707287 | 464_88971_3815(KIT)_6 | 0 | — |
| chr4 | 54709423 | 54709539 | 464_88972_3815(KIT)_7 | 0 | — |
| chr4 | 54723583 | 54723698 | 464_88973_3815(KIT)_8 | 0 | — |
| chr4 | 54725856 | 54726050 | 464_88974_3815(KIT)_9 | 0 | — |
| chr4 | 54727217 | 54727324 | 464_88975_3815(KIT)_10 | 0 | — |
| chr4 | 54727415 | 54727542 | 464_88976_3815(KIT)_11 | 0 | — |
| chr4 | 54727822 | 54727927 | 464_88977_3815(KIT)_12 | 0 | — |
| chr4 | 54728010 | 54728121 | 464_88978_3815(KIT)_13 | 0 | — |
| chr4 | 54729334 | 54729485 | 464_88979_3815(KIT)_14 | 0 | — |
| chr4 | 54731327 | 54731419 | 464_88980_3815(KIT)_15 | 0 | — |
| chr4 | 54731870 | 54731998 | 464_88981_3815(KIT)_16 | 0 | — |
| chr4 | 54733069 | 54733192 | 464_88982_3815(KIT)_17 | 0 | — |
| chr4 | 54736497 | 54736609 | 464_88983_3815(KIT)_18 | 0 | — |
| chr4 | 54736720 | 54736820 | 464_88984_3815(KIT)_19 | 0 | — |
| chr4 | 54737174 | 54737280 | 464_88985_3815(KIT)_20 | 0 | — |
| chr4 | 54738428 | 54738557 | 464_88986_3815(KIT)_21 | 0 | — |
| chr4 | 55079940 | 55080163 | 464_88725_3791(KDR)_30a | 0 | — |
| chr4 | 55081955 | 55082041 | 464_88724_3791(KDR)_29a | 0 | — |
| chr4 | 55082535 | 55082635 | 464_88723_3791(KDR)_28a | 0 | — |
| chr4 | 55087606 | 55087758 | 464_88722_3791(KDR)_27a | 0 | — |
| chr4 | 55088867 | 55088973 | 464_88721_3791(KDR)_26a | 0 | — |
| chr4 | 55089373 | 55089473 | 464_88720_3791(KDR)_25a | 0 | — |
| chr4 | 55089690 | 55089802 | 464_88719_3791(KDR)_24a | 0 | — |
| chr4 | 55089955 | 55090078 | 464_88718_3791(KDR)_23a | 0 | — |
| chr4 | 55092616 | 55092714 | 464_88717_3791(KDR)_22a | 0 | — |
| chr4 | 55094801 | 55094955 | 464_88716_3791(KDR)_21a | 0 | — |
| chr4 | 55095576 | 55095665 | 464_88715_3791(KDR)_20a | 0 | — |
| chr4 | 55096228 | 55096342 | 464_88714_3791(KDR)_19a | 0 | — |
| chr4 | 55097661 | 55097766 | 464_88713_3791(KDR)_18a | 0 | — |
| chr4 | 55098136 | 55098272 | 464_88712_3791(KDR)_17a | 0 | — |
| chr4 | 55098696 | 55098803 | 464_88711_3791(KDR)_16a | 0 | — |
| chr4 | 55101896 | 55102028 | 464_88710_3791(KDR)_15a | 0 | — |
| chr4 | 55102361 | 55102508 | 464_88709_3791(KDR)_14a | 0 | — |
| chr4 | 55104642 | 55104984 | 464_88708_3791(KDR)_13a | 0 | — |
| chr4 | 55105831 | 55105940 | 464_88707_3791(KDR)_12a | 0 | — |
| chr4 | 55106686 | 55106810 | 464_88706_3791(KDR)_11a | 0 | — |
| chr4 | 55107736 | 55107893 | 464_88705_3791(KDR)_10a | 0 | — |
| chr4 | 55110402 | 55110566 | 464_88704_3791(KDR)_9a | 0 | — |
| chr4 | 55110653 | 55110768 | 464_88703_3791(KDR)_8a | 0 | — |
| chr4 | 55113303 | 55113481 | 464_88702_3791(KDR)_7a | 0 | — |
| chr4 | 55114125 | 55114265 | 464_88701_3791(KDR)_6a | 0 | — |
| chr4 | 55114873 | 55115042 | 464_88700_3791(KDR)_5a | 0 | — |
| chr4 | 55115280 | 55115411 | 464_88699_3791(KDR)_4a | 0 | — |
| chr4 | 55118603 | 55118800 | 464_88698_3791(KDR)_3a | 0 | — |
| chr4 | 55121096 | 55121190 | 464_88697_3791(KDR)_2a | 0 | — |
| chr4 | 55125226 | 55125293 | 464_88696_3791(KDR)_1a | 0 | — |
| chr4 | 152323088 | 152323149 | 461_64096_55294(FBXW7)_17 | 0 | — |
| chr4 | 152324183 | 152324394 | 461_64095_55294(FBXW7)_16 | 0 | — |
| chr4 | 152326005 | 152326231 | 461_64094_55294(FBXW7)_15 | 0 | — |
| chr4 | 152328207 | 152328389 | 461_64093_55294(FBXW7)_14 | 0 | — |
| chr4 | 152329671 | 152329785 | 461_64092_55294(FBXW7)_13 | 0 | — |
| chr4 | 152330731 | 152330868 | 461_64091_55294(FBXW7)_12 | 0 | — |
| chr4 | 152332595 | 152332719 | 461_64090_55294(FBXW7)_11 | 0 | — |
| chr4 | 152337801 | 152337936 | 461_64089_55294(FBXW7)_10 | 0 | — |
| chr4 | 152346929 | 152347071 | 461_64088_55294(FBXW7)_9 | 0 | — |
| chr4 | 152348673 | 152348801 | 461_64087_55294(FBXW7)_8 | 0 | — |
| chr4 | 152350041 | 152350124 | 461_64086_55294(FBXW7)_7 | 0 | — |
| chr4 | 152352469 | 152352958 | 461_64085_55294(FBXW7)_6 | 0 | — |
| chr4 | 152382188 | 152382512 | 461_64084_55294(FBXW7)_5 | 0 | — |
| chr4 | 152411159 | 152411872 | 461_64083_55294(FBXW7)_4 | 0 | — |
| chr5 | 56815573 | 56816055 | 465_99617_4214(MAP3K1)_1a | 0 | — |
| chr5 | 56817055 | 56817116 | 465_99618_4214(MAP3K1)_2a | 0 | — |
| chr5 | 56820391 | 56820816 | 465_99619_4214(MAP3K1)_3a | 0 | — |
| chr5 | 56856599 | 56856750 | 465_99620_4214(MAP3K1)_4a | 0 | — |
| chr5 | 56859714 | 56859915 | 465_99621_4214(MAP3K1)_5a | 0 | — |
| chr5 | 56864733 | 56864934 | 465_99622_4214(MAP3K1)_6a | 0 | — |
| chr5 | 56865339 | 56865456 | 465_99623_4214(MAP3K1)_7a | 0 | — |
| chr5 | 56865828 | 56865977 | 465_99624_4214(MAP3K1)_8a | 0 | — |
| chr5 | 56871909 | 56872031 | 465_99625_4214(MAP3K1)_9a | 0 | — |
| chr5 | 56872640 | 56872722 | 465_99626_4214(MAP3K1)_10a | 0 | — |
| chr5 | 56872824 | 56873005 | 465_99627_4214(MAP3K1)_11a | 0 | — |
| chr5 | 56875031 | 56875310 | 465_99628_4214(MAP3K1)_12a | 0 | — |
| chr5 | 56878979 | 56879101 | 465_99629_4214(MAP3K1)_13a | 0 | — |
| chr5 | 56880710 | 56880802 | 465_99630_4214(MAP3K1)_14a | 0 | — |
| chr5 | 56881082 | 56881272 | 465_99631_4214(MAP3K1)_15a | 0 | — |
| chr5 | 56881569 | 56882866 | 465_99632_4214(MAP3K1)_16a | 0 | — |
| chr5 | 56883526 | 56883679 | 465_99633_4214(MAP3K1)_17a | 0 | — |
| chr5 | 56884663 | 56884826 | 465_99634_4214(MAP3K1)_18a | 0 | — |
| chr5 | 56885931 | 56886063 | 465_99635_4214(MAP3K1)_19a | 0 | — |
| chr5 | 56887377 | 56887520 | 465_99636_4214(MAP3K1)_20a | 0 | — |
| chr5 | 56888225 | 56888357 | 465_99637_4214(MAP3K1)_21a | 0 | — |
| chr5 | 56893530 | 56893680 | 465_99638_4214(MAP3K1)_22a | 0 | — |
| chr5 | 68226675 | 68227009 | 470_127774_5295(PIK3R1)_2 | 0 | — |
| chr5 | 68273389 | 68273482 | 470_127775_5295(PIK3R1)_3 | 0 | — |
| chr5 | 68273938 | 68274013 | 470_127776_5295(PIK3R1)_4 | 0 | — |
| chr5 | 68279601 | 68279733 | 470_127777_5295(PIK3R1)_5 | 0 | — |
| chr5 | 68280527 | 68280729 | 470_127778_5295(PIK3R1)_6 | 0 | — |
| chr5 | 68280926 | 68281006 | 470_127779_5295(PIK3R1)_7 | 0 | — |
| chr5 | 68288423 | 68288751 | 470_127780_5295(PIK3R1)_8 | 0 | — |
| chr5 | 68290638 | 68290834 | 470_127781_5295(PIK3R1)_9 | 0 | — |
| chr5 | 68292258 | 68292361 | 470_127782_5295(PIK3R1)_10 | 0 | — |
| chr5 | 68292567 | 68292729 | 470_127783_5295(PIK3R1)_11 | 0 | — |
| chr5 | 68293100 | 68293199 | 470_127784_5295(PIK3R1)_12 | 0 | — |
| chr5 | 68293302 | 68293483 | 470_127785_5295(PIK3R1)_13 | 0 | — |
| chr5 | 68293708 | 68293834 | 470_127786_5295(PIK3R1)_14 | 0 | — |
| chr5 | 68294535 | 68294678 | 470_127787_5295(PIK3R1)_15 | 0 | — |
| chr5 | 68295147 | 68295324 | 470_127788_5295(PIK3R1)_16 | 0 | — |
| chr5 | 68295419 | 68295488 | 470_127789_5295(PIK3R1)_17 | 0 | — |
| chr5 | 68296170 | 68296341 | 470_127790_5295(PIK3R1)_18 | 0 | — |
| chr5 | 68297411 | 68297601 | 470_127791_5295(PIK3R1)_19 | 0 | — |
| chr5 | 112707717 | 112707882 | 426_2903_324(APC)_1a | 0 | — |
| chr5 | 112737858 | 112737925 | 426_2904_324(APC)_2a | 0 | — |
| chr5 | 112738331 | 112738460 | 426_2905_324(APC)_3a | 0 | — |
| chr5 | 112754872 | 112755025 | 426_2906_324(APC)_4a | 0 | — |
| chr5 | 112755074 | 112755094 | 426_2907_324(APC)_5a | 0 | — |
| chr5 | 112766325 | 112766410 | 426_2908_324(APC)_6a | 0 | — |
| chr5 | 112767188 | 112767390 | 426_2909_324(APC)_7a | 0 | — |
| chr5 | 112775628 | 112775737 | 426_2910_324(APC)_8a | 0 | — |
| chr5 | 112780789 | 112780903 | 426_2911_324(APC)_9a | 0 | — |
| chr5 | 112792445 | 112792529 | 426_2912_324(APC)_10a | 0 | — |
| chr5 | 112801278 | 112801383 | 426_2913_324(APC)_11a | 0 | — |
| chr5 | 112815494 | 112815593 | 426_2914_324(APC)_12a | 0 | — |
| chr5 | 112818965 | 112819344 | 426_2915_324(APC)_13a | 0 | — |
| chr5 | 112821895 | 112821991 | 426_2916_324(APC)_14a | 0 | — |
| chr5 | 112827107 | 112827247 | 426_2917_324(APC)_15a | 0 | — |
| chr5 | 112827928 | 112828006 | 426_2918_324(APC)_16a | 0 | — |
| chr5 | 112828855 | 112828972 | 426_2919_324(APC)_17a | 0 | — |
| chr5 | 112834950 | 112835165 | 426_2920_324(APC)_18a | 0 | — |
| chr5 | 112837552 | 112844126 | 426_2921_324(APC)_19a | 0 | — |
| chr5 | 150054068 | 150054224 | 429_36473_1436(CSF1R)_22a | 0 | — |
| chr5 | 150054321 | 150054430 | 429_36472_1436(CSF1R)_21a | 0 | — |
| chr5 | 150055236 | 150055336 | 429_36471_1436(CSF1R)_20a | 0 | — |
| chr5 | 150056025 | 150056137 | 429_36470_1436(CSF1R)_19a | 0 | — |
| chr5 | 150056218 | 150056341 | 429_36469_1436(CSF1R)_18a | 0 | — |
| chr5 | 150057286 | 150057384 | 429_36468_1436(CSF1R)_17a | 0 | — |
| chr5 | 150057503 | 150057592 | 429_36467_1436(CSF1R)_16a | 0 | — |
| chr5 | 150059699 | 150059862 | 429_36466_1436(CSF1R)_15a | 0 | — |
| chr5 | 150060861 | 150060972 | 429_36465_1436(CSF1R)_14a | 0 | — |
| chr5 | 150061490 | 150061595 | 429_36464_1436(CSF1R)_13a | 0 | — |
| chr5 | 150061722 | 150061849 | 429_36463_1436(CSF1R)_12a | 0 | — |
| chr5 | 150068214 | 150068330 | 429_36462_1436(CSF1R)_11a | 0 | — |
| chr5 | 150069872 | 150070063 | 429_36461_1436(CSF1R)_10a | 0 | — |
| chr5 | 150070181 | 150070302 | 429_36460_1436(CSF1R)_9a | 0 | — |
| chr5 | 150070455 | 150070571 | 429_36459_1436(CSF1R)_8a | 0 | — |
| chr5 | 150073300 | 150073493 | 429_36458_1436(CSF1R)_7a | 0 | — |
| chr5 | 150077275 | 150077435 | 429_36457_1436(CSF1R)_6a | 0 | — |
| chr5 | 150078111 | 150078248 | 429_36456_1436(CSF1R)_5a | 0 | — |
| chr5 | 150080051 | 150080336 | 429_36455_1436(CSF1R)_4a | 0 | — |
| chr5 | 150080766 | 150081024 | 429_36454_1436(CSF1R)_3a | 0 | — |
| chr5 | 150086378 | 150086427 | 429_36453_1436(CSF1R)_2a | 0 | — |
| chr5 | 171387948 | 171388006 | 469_119415_4869(NPM1)_1 | 0 | — |
| chr5 | 171390050 | 171390130 | 469_119416_4869(NPM1)_2 | 0 | — |
| chr5 | 171391304 | 171391424 | 469_119417_4869(NPM1)_3 | 0 | — |
| chr5 | 171391705 | 171391799 | 469_119418_4869(NPM1)_4 | 0 | — |
| chr5 | 171392709 | 171392816 | 469_119419_4869(NPM1)_5 | 0 | — |
| chr5 | 171392913 | 171392978 | 469_119420_4869(NPM1)_6 | 0 | — |
| chr5 | 171400152 | 171400210 | 469_119421_4869(NPM1)_7 | 0 | — |
| chr5 | 171400838 | 171400925 | 469_119422_4869(NPM1)_8 | 0 | — |
| chr5 | 171405301 | 171405403 | 469_119423_4869(NPM1)_9 | 0 | — |
| chr5 | 171406396 | 171406734 | 469_119424_4869(NPM1)_10 | 0 | — |
| chr5 | 171407699 | 171407774 | 469_119425_4869(NPM1)_11 | 0 | — |
| chr5 | 171410526 | 171410565 | 469_119426_4869(NPM1)_12 | 0 | — |
| chr5 | 179698372 | 179698603 | 428_24377_821(CANX)_1 | 0 | — |
| chr5 | 179698928 | 179699102 | 428_24378_821(CANX)_2 | 0 | — |
| chr5 | 179705678 | 179705852 | 428_24379_821(CANX)_3 | 0 | — |
| chr5 | 179706257 | 179706331 | 428_24380_821(CANX)_4 | 0 | — |
| chr5 | 179707131 | 179707190 | 428_24381_821(CANX)_5 | 0 | — |
| chr5 | 179708238 | 179708380 | 428_24382_821(CANX)_6 | 0 | — |
| chr5 | 179708977 | 179709059 | 428_24383_821(CANX)_7 | 0 | — |
| chr5 | 179709872 | 179710065 | 428_24384_821(CANX)_8 | 0 | — |
| chr5 | 179716104 | 179716294 | 428_24385_821(CANX)_9 | 0 | — |
| chr5 | 179719667 | 179719781 | 428_24386_821(CANX)_10 | 0 | — |
| chr5 | 179720403 | 179720560 | 428_24387_821(CANX)_11 | 0 | — |
| chr5 | 179722803 | 179723019 | 428_24388_821(CANX)_12 | 0 | — |
| chr5 | 179723659 | 179723779 | 428_24389_821(CANX)_13 | 0 | — |
| chr5 | 179724656 | 179724783 | 428_24390_821(CANX)_14 | 0 | — |
| chr5 | 179726679 | 179726759 | 428_24391_821(CANX)_15 | 0 | — |
| chr5 | 179728590 | 179728644 | 428_24392_821(CANX)_16 | 0 | — |
| chr6 | 29942553 | 29942626 | 463_78379_3105(HLA-A)_1 | 0 | — |
| chr6 | 29942756 | 29943026 | 463_78380_3105(HLA-A)_2 | 0 | — |
| chr6 | 29943267 | 29943543 | 463_78381_3105(HLA-A)_3 | 0 | — |
| chr6 | 29944121 | 29944397 | 463_78382_3105(HLA-A)_4 | 0 | — |
| chr6 | 29944499 | 29944616 | 463_78383_3105(HLA-A)_5 | 0 | — |
| chr6 | 29945058 | 29945091 | 463_78384_3105(HLA-A)_6 | 0 | — |
| chr6 | 29945233 | 29945281 | 463_78385_3105(HLA-A)_7 | 0 | — |
| chr6 | 29945450 | 29945455 | 463_78386_3105(HLA-A)_8 | 0 | — |
| chr6 | 32195436 | 32196421 | 469_119235_4855(NOTCH4)_29 | 0 | — |
| chr6 | 32196924 | 32197072 | 469_119234_4855(NOTCH4)_28 | 0 | — |
| chr6 | 32197298 | 32197594 | 469_119233_4855(NOTCH4)_27 | 0 | — |
| chr6 | 32198420 | 32198559 | 469_119232_4855(NOTCH4)_26 | 0 | — |
| chr6 | 32198648 | 32198730 | 469_119231_4855(NOTCH4)_25 | 0 | — |
| chr6 | 32198925 | 32199145 | 469_119230_4855(NOTCH4)_24 | 0 | — |
| chr6 | 32200830 | 32201006 | 469_119229_4855(NOTCH4)_23 | 0 | — |
| chr6 | 32201116 | 32201500 | 469_119228_4855(NOTCH4)_22 | 0 | — |
| chr6 | 32202075 | 32202599 | 469_119227_4855(NOTCH4)_21 | 0 | — |
| chr6 | 32203769 | 32203882 | 469_119226_4855(NOTCH4)_20 | 0 | — |
| chr6 | 32204136 | 32204389 | 469_119225_4855(NOTCH4)_19 | 0 | — |
| chr6 | 32210751 | 32210936 | 469_119224_4855(NOTCH4)_18 | 0 | — |
| chr6 | 32212473 | 32212627 | 469_119223_4855(NOTCH4)_17 | 0 | — |
| chr6 | 32212823 | 32212911 | 469_119222_4855(NOTCH4)_16 | 0 | — |
| chr6 | 32213134 | 32213252 | 469_119221_4855(NOTCH4)_15 | 0 | — |
| chr6 | 32213687 | 32213840 | 469_119220_4855(NOTCH4)_14 | 0 | — |
| chr6 | 32214109 | 32214255 | 469_119219_4855(NOTCH4)_13 | 0 | — |
| chr6 | 32215225 | 32215385 | 469_119218_4855(NOTCH4)_12 | 0 | — |
| chr6 | 32216944 | 32217067 | 469_119217_4855(NOTCH4)_11 | 0 | — |
| chr6 | 32217152 | 32217266 | 469_119216_4855(NOTCH4)_10 | 0 | — |
| chr6 | 32217994 | 32218108 | 469_119215_4855(NOTCH4)_9 | 0 | — |
| chr6 | 32219591 | 32219786 | 469_119214_4855(NOTCH4)_8 | 0 | — |
| chr6 | 32220128 | 32220284 | 469_119213_4855(NOTCH4)_7 | 0 | — |
| chr6 | 32220404 | 32220641 | 469_119212_4855(NOTCH4)_6 | 0 | — |
| chr6 | 32220755 | 32220878 | 469_119211_4855(NOTCH4)_5 | 0 | — |
| chr6 | 32220977 | 32221325 | 469_119210_4855(NOTCH4)_4 | 0 | — |
| chr6 | 32222510 | 32222806 | 469_119209_4855(NOTCH4)_3 | 0 | — |
| chr6 | 32223004 | 32223086 | 469_119208_4855(NOTCH4)_2 | 0 | — |
| chr6 | 32223855 | 32223928 | 469_119207_4855(NOTCH4)_1 | 0 | — |
| chr6 | 32845578 | 32845785 | 475_171283_6890(TAP1)_12 | 0 | — |
| chr6 | 32847067 | 32847204 | 475_171282_6890(TAP1)_11 | 0 | — |
| chr6 | 32847512 | 32847675 | 475_171281_6890(TAP1)_10 | 0 | — |
| chr6 | 32847918 | 32848092 | 475_171280_6890(TAP1)_9 | 0 | — |
| chr6 | 32848651 | 32848840 | 475_171279_6890(TAP1)_8 | 0 | — |
| chr6 | 32848989 | 32849118 | 475_171278_6890(TAP1)_7 | 0 | — |
| chr6 | 32850319 | 32850517 | 475_171277_6890(TAP1)_6 | 0 | — |
| chr6 | 32850943 | 32851149 | 475_171276_6890(TAP1)_5 | 0 | — |
| chr6 | 32852108 | 32852239 | 475_171275_6890(TAP1)_4 | 0 | — |
| chr6 | 32852387 | 32852502 | 475_171274_6890(TAP1)_3 | 0 | — |
| chr6 | 32852594 | 32852782 | 475_171273_6890(TAP1)_2 | 0 | — |
| chr6 | 32853038 | 32853816 | 475_171272_6890(TAP1)_1 | 0 | — |
| chr6 | 33301759 | 33301771 | 475_171303_6892(TAPBP)_8 | 0 | — |
| chr6 | 33303571 | 33303989 | 475_171302_6892(TAPBP)_7 | 0 | — |
| chr6 | 33304126 | 33304217 | 475_171301_6892(TAPBP)_6 | 0 | — |
| chr6 | 33304295 | 33304638 | 475_171300_6892(TAPBP)_5 | 0 | — |
| chr6 | 33304987 | 33305387 | 475_171299_6892(TAPBP)_4 | 0 | — |
| chr6 | 33313216 | 33313477 | 475_171298_6892(TAPBP)_3 | 0 | — |
| chr6 | 33313692 | 33313864 | 475_171297_6892(TAPBP)_2 | 0 | — |
| chr6 | 33314003 | 33314041 | 475_171296_6892(TAPBP)_1 | 0 | — |
| chr6 | 151807912 | 151808364 | 461_58879_2099(ESR1)_5 | 0 | — |
| chr6 | 151842596 | 151842793 | 461_58880_2099(ESR1)_6 | 0 | — |
| chr6 | 151880654 | 151880771 | 461_58881_2099(ESR1)_7 | 0 | — |
| chr6 | 151944172 | 151944508 | 461_58882_2099(ESR1)_8 | 0 | — |
| chr6 | 152011655 | 152011794 | 461_58883_2099(ESR1)_9 | 0 | — |
| chr6 | 152060990 | 152061124 | 461_58884_2099(ESR1)_10 | 0 | — |
| chr6 | 152094384 | 152094568 | 461_58885_2099(ESR1)_11 | 0 | — |
| chr6 | 152098731 | 152098966 | 461_58886_2099(ESR1)_12 | 0 | — |
| chr7 | 44114784 | 44114945 | 470_129130_5425(POLD2)_13 | 0 | — |
| chr7 | 44115294 | 44115396 | 470_129129_5425(POLD2)_12 | 0 | — |
| chr7 | 44115765 | 44115893 | 470_129128_5425(POLD2)_11 | 0 | — |
| chr7 | 44116114 | 44116272 | 470_129127_5425(POLD2)_10 | 0 | — |
| chr7 | 44116429 | 44116510 | 470_129126_5425(POLD2)_9 | 0 | — |
| chr7 | 44116816 | 44117015 | 470_129125_5425(POLD2)_8 | 0 | — |
| chr7 | 44117132 | 44117247 | 470_129124_5425(POLD2)_7 | 0 | — |
| chr7 | 44117618 | 44117742 | 470_129123_5425(POLD2)_6 | 0 | — |
| chr7 | 44117942 | 44118064 | 470_129122_5425(POLD2)_5 | 0 | — |
| chr7 | 44121833 | 44122459 | 470_129121_5425(POLD2)_4 | 0 | — |
| chr7 | 44123178 | 44123508 | 470_129120_5425(POLD2)_3 | 0 | — |
| chr7 | 44123510 | 44123570 | 470_129119_5425(POLD2)_2 | 0 | — |
| chr7 | 44123612 | 44123657 | 470_129118_5425(POLD2)_1 | 0 | — |
| chr7 | 55019277 | 55019365 | 431_48541_1956(EGFR)_1 | 0 | — |
| chr7 | 55109722 | 55109958 | 431_48542_1956(EGFR)_2 | 0 | — |
| chr7 | 55142285 | 55142437 | 431_48543_1956(EGFR)_3 | 0 | — |
| chr7 | 55143304 | 55143488 | 431_48544_1956(EGFR)_4 | 0 | — |
| chr7 | 55146605 | 55146740 | 431_48545_1956(EGFR)_5 | 0 | — |
| chr7 | 55151293 | 55151362 | 431_48546_1956(EGFR)_6 | 0 | — |
| chr7 | 55152545 | 55152664 | 431_48547_1956(EGFR)_7 | 0 | — |
| chr7 | 55154010 | 55154152 | 431_48548_1956(EGFR)_8 | 0 | — |
| chr7 | 55155829 | 55155946 | 431_48549_1956(EGFR)_9 | 0 | — |
| chr7 | 55156532 | 55156659 | 431_48550_1956(EGFR)_10 | 0 | — |
| chr7 | 55156758 | 55156951 | 431_48551_1956(EGFR)_11 | 0 | — |
| chr7 | 55157662 | 55157753 | 431_48552_1956(EGFR)_12 | 0 | — |
| chr7 | 55160138 | 55160338 | 431_48553_1956(EGFR)_13 | 0 | — |
| chr7 | 55161498 | 55161631 | 431_48554_1956(EGFR)_14 | 0 | — |
| chr7 | 55163732 | 55163823 | 431_48555_1956(EGFR)_15 | 0 | — |
| chr7 | 55165279 | 55165437 | 431_48556_1956(EGFR)_16 | 0 | — |
| chr7 | 55168522 | 55168635 | 431_48557_1956(EGFR)_17 | 0 | — |
| chr7 | 55170306 | 55171045 | 431_48558_1956(EGFR)_18 | 0 | — |
| chr7 | 55171174 | 55171213 | 431_48559_1956(EGFR)_19 | 0 | — |
| chr7 | 55172845 | 55172957 | 431_48560_1956(EGFR)_20 | 0 | — |
| chr7 | 55172982 | 55173124 | 431_48561_1956(EGFR)_21 | 0 | — |
| chr7 | 55173920 | 55174043 | 431_48562_1956(EGFR)_22 | 0 | — |
| chr7 | 55174721 | 55174820 | 431_48563_1956(EGFR)_23 | 0 | — |
| chr7 | 55181292 | 55181478 | 431_48564_1956(EGFR)_24 | 0 | — |
| chr7 | 55191718 | 55191874 | 431_48565_1956(EGFR)_25 | 0 | — |
| chr7 | 55192765 | 55192841 | 431_48566_1956(EGFR)_26 | 0 | — |
| chr7 | 55198716 | 55198863 | 431_48567_1956(EGFR)_27 | 0 | — |
| chr7 | 55200315 | 55200413 | 431_48568_1956(EGFR)_28 | 0 | — |
| chr7 | 55201187 | 55201355 | 431_48569_1956(EGFR)_29 | 0 | — |
| chr7 | 55201734 | 55201782 | 431_48570_1956(EGFR)_30 | 0 | — |
| chr7 | 55202516 | 55202625 | 431_48571_1956(EGFR)_31 | 0 | — |
| chr7 | 55205255 | 55205617 | 431_48572_1956(EGFR)_32 | 0 | — |
| chr7 | 101020135 | 101020217 | 466_116431_140453(MUC17)_1 | 0 | — |
| chr7 | 101031119 | 101031221 | 466_116432_140453(MUC17)_2 | 0 | — |
| chr7 | 101031600 | 101043819 | 466_116433_140453(MUC17)_3 | 0 | — |
| chr7 | 101047983 | 101048115 | 466_116434_140453(MUC17)_4 | 0 | — |
| chr7 | 101048844 | 101048972 | 466_116435_140453(MUC17)_5 | 0 | — |
| chr7 | 101049323 | 101049382 | 466_116436_140453(MUC17)_6 | 0 | — |
| chr7 | 101050483 | 101050635 | 466_116437_140453(MUC17)_7 | 0 | — |
| chr7 | 101051612 | 101051681 | 466_116438_140453(MUC17)_8 | 0 | — |
| chr7 | 101051802 | 101051962 | 466_116439_140453(MUC17)_9 | 0 | — |
| chr7 | 101052985 | 101053147 | 466_116440_140453(MUC17)_10 | 0 | — |
| chr7 | 101053338 | 101053436 | 466_116441_140453(MUC17)_11 | 0 | — |
| chr7 | 101056193 | 101056270 | 466_116442_140453(MUC17)_12 | 0 | — |
| chr7 | 101058002 | 101058044 | 466_116443_140453(MUC17)_13 | 0 | — |
| chr7 | 116695756 | 116695799 | 466_106278_4233(MET)_2a | 0 | — |
| chr7 | 116699070 | 116700284 | 466_106279_4233(MET)_3a | 0 | — |
| chr7 | 116724099 | 116724164 | 466_106280_4233(MET)_4a | 0 | — |
| chr7 | 116724771 | 116724847 | 466_106281_4233(MET)_5a | 0 | — |
| chr7 | 116731667 | 116731859 | 466_106282_4233(MET)_6a | 0 | — |
| chr7 | 116739949 | 116740084 | 466_106283_4233(MET)_7a | 0 | — |
| chr7 | 116740851 | 116741025 | 466_106284_4233(MET)_8a | 0 | — |
| chr7 | 116755354 | 116755515 | 466_106285_4233(MET)_9a | 0 | — |
| chr7 | 116757436 | 116757539 | 466_106286_4233(MET)_10a | 0 | — |
| chr7 | 116757637 | 116757774 | 466_106287_4233(MET)_11a | 0 | — |
| chr7 | 116758458 | 116758620 | 466_106288_4233(MET)_12a | 0 | — |
| chr7 | 116759336 | 116759490 | 466_106289_4233(MET)_13a | 0 | — |
| chr7 | 116763049 | 116763268 | 466_106290_4233(MET)_14a | 0 | — |
| chr7 | 116769644 | 116769791 | 466_106291_4233(MET)_15a | 0 | — |
| chr7 | 116771497 | 116771654 | 466_106292_4233(MET)_16a | 0 | — |
| chr7 | 116771848 | 116771989 | 466_106293_4233(MET)_17a | 0 | — |
| chr7 | 116774880 | 116775111 | 466_106294_4233(MET)_18a | 0 | — |
| chr7 | 116777388 | 116777469 | 466_106295_4233(MET)_19a | 0 | — |
| chr7 | 116778775 | 116778957 | 466_106296_4233(MET)_20a | 0 | — |
| chr7 | 116781987 | 116782097 | 466_106297_4233(MET)_21a | 0 | — |
| chr7 | 116783303 | 116783469 | 466_106298_4233(MET)_22a | 0 | — |
| chr7 | 116795654 | 116795791 | 466_106299_4233(MET)_23a | 0 | — |
| chr7 | 116795886 | 116796124 | 466_106300_4233(MET)_24a | 0 | — |
| chr7 | 129189151 | 129189482 | 474_160564_6608(SMO)_1a | 0 | — |
| chr7 | 129203383 | 129203589 | 474_160565_6608(SMO)_2a | 0 | — |
| chr7 | 129205202 | 129205412 | 474_160566_6608(SMO)_3a | 0 | — |
| chr7 | 129205609 | 129205782 | 474_160567_6608(SMO)_4a | 0 | — |
| chr7 | 129206149 | 129206369 | 474_160568_6608(SMO)_5a | 0 | — |
| chr7 | 129206463 | 129206587 | 474_160569_6608(SMO)_6a | 0 | — |
| chr7 | 129208758 | 129208851 | 474_160570_6608(SMO)_7a | 0 | — |
| chr7 | 129209288 | 129209397 | 474_160571_6608(SMO)_8a | 0 | — |
| chr7 | 129210362 | 129210548 | 474_160572_6608(SMO)_9a | 0 | — |
| chr7 | 129210964 | 129211113 | 474_160573_6608(SMO)_10a | 0 | — |
| chr7 | 129211635 | 129211770 | 474_160574_6608(SMO)_11a | 0 | — |
| chr7 | 129212023 | 129212451 | 474_160575_6608(SMO)_12a | 0 | — |
| chr7 | 140719333 | 140726516 | 427_15649_673(BRAF)_20 | 0 | — |
| chr7 | 140734011 | 140734770 | 427_15648_673(BRAF)_19 | 0 | — |
| chr7 | 140739811 | 140739946 | 427_15647_673(BRAF)_18 | 0 | — |
| chr7 | 140747365 | 140747447 | 427_15646_673(BRAF)_17 | 0 | — |
| chr7 | 140749286 | 140749418 | 427_15645_673(BRAF)_16 | 0 | — |
| chr7 | 140753274 | 140753393 | 427_15644_673(BRAF)_15 | 0 | — |
| chr7 | 140754186 | 140754233 | 427_15643_673(BRAF)_14 | 0 | — |
| chr7 | 140776911 | 140777088 | 427_15642_673(BRAF)_13 | 0 | — |
| chr7 | 140777990 | 140778075 | 427_15641_673(BRAF)_12 | 0 | — |
| chr7 | 140781575 | 140781693 | 427_15640_673(BRAF)_11 | 0 | — |
| chr7 | 140783020 | 140783157 | 427_15639_673(BRAF)_10 | 0 | — |
| chr7 | 140787547 | 140787584 | 427_15638_673(BRAF)_9 | 0 | — |
| chr7 | 140794307 | 140794467 | 427_15637_673(BRAF)_8 | 0 | — |
| chr7 | 140800361 | 140800481 | 427_15636_673(BRAF)_7 | 0 | — |
| chr7 | 140801411 | 140801560 | 427_15635_673(BRAF)_6 | 0 | — |
| chr7 | 140807959 | 140808062 | 427_15634_673(BRAF)_5 | 0 | — |
| chr7 | 140808891 | 140808995 | 427_15633_673(BRAF)_4 | 0 | — |
| chr7 | 140834608 | 140834872 | 427_15632_673(BRAF)_3 | 0 | — |
| chr7 | 140850110 | 140850212 | 427_15631_673(BRAF)_2 | 0 | — |
| chr7 | 140924565 | 140924756 | 427_15630_673(BRAF)_1 | 0 | — |
| chr7 | 148807645 | 148807706 | 461_59365_2146(EZH2)_22 | 0 | — |
| chr7 | 148809070 | 148809155 | 461_59364_2146(EZH2)_21 | 0 | — |
| chr7 | 148809309 | 148809390 | 461_59363_2146(EZH2)_20 | 0 | — |
| chr7 | 148810332 | 148810414 | 461_59362_2146(EZH2)_19 | 0 | — |
| chr7 | 148811624 | 148811720 | 461_59361_2146(EZH2)_18 | 0 | — |
| chr7 | 148813958 | 148814137 | 461_59360_2146(EZH2)_17 | 0 | — |
| chr7 | 148814913 | 148815039 | 461_59359_2146(EZH2)_16 | 0 | — |
| chr7 | 148815505 | 148815546 | 461_59358_2146(EZH2)_15 | 0 | — |
| chr7 | 148816683 | 148816778 | 461_59357_2146(EZH2)_14 | 0 | — |
| chr7 | 148817221 | 148817391 | 461_59356_2146(EZH2)_13 | 0 | — |
| chr7 | 148817876 | 148818117 | 461_59355_2146(EZH2)_12 | 0 | — |
| chr7 | 148819595 | 148819687 | 461_59354_2146(EZH2)_11 | 0 | — |
| chr7 | 148826453 | 148826632 | 461_59353_2146(EZH2)_10 | 0 | — |
| chr7 | 148827163 | 148827266 | 461_59352_2146(EZH2)_9 | 0 | — |
| chr7 | 148828739 | 148828880 | 461_59351_2146(EZH2)_8 | 0 | — |
| chr7 | 148829727 | 148829848 | 461_59350_2146(EZH2)_7 | 0 | — |
| chr7 | 148832633 | 148832750 | 461_59349_2146(EZH2)_6 | 0 | — |
| chr7 | 148846469 | 148846598 | 461_59348_2146(EZH2)_5 | 0 | — |
| chr7 | 148847181 | 148847305 | 461_59347_2146(EZH2)_4 | 0 | — |
| chr7 | 148850453 | 148850470 | 461_59346_2146(EZH2)_3 | 0 | — |
| chr7 | 152136831 | 152136924 | 464_95024_58508(KMT2C)_61 | 0 | — |
| chr7 | 152138795 | 152138904 | 464_95023_58508(KMT2C)_60 | 0 | — |
| chr7 | 152139185 | 152139259 | 464_95022_58508(KMT2C)_59 | 0 | — |
| chr7 | 152139674 | 152139791 | 464_95021_58508(KMT2C)_58 | 0 | — |
| chr7 | 152144712 | 152144881 | 464_95020_58508(KMT2C)_57 | 0 | — |
| chr7 | 152145152 | 152145295 | 464_95019_58508(KMT2C)_56 | 0 | — |
| chr7 | 152146598 | 152146735 | 464_95018_58508(KMT2C)_55 | 0 | — |
| chr7 | 152148032 | 152149152 | 464_95017_58508(KMT2C)_54 | 0 | — |
| chr7 | 152150899 | 152151007 | 464_95016_58508(KMT2C)_53 | 0 | — |
| chr7 | 152151441 | 152151581 | 464_95015_58508(KMT2C)_52 | 0 | — |
| chr7 | 152152704 | 152152954 | 464_95014_58508(KMT2C)_51 | 0 | — |
| chr7 | 152154009 | 152154146 | 464_95013_58508(KMT2C)_50 | 0 | — |
| chr7 | 152154266 | 152154445 | 464_95012_58508(KMT2C)_49 | 0 | — |
| chr7 | 152155909 | 152156057 | 464_95011_58508(KMT2C)_48 | 0 | — |
| chr7 | 152156204 | 152156346 | 464_95010_58508(KMT2C)_47 | 0 | — |
| chr7 | 152157760 | 152157925 | 464_95009_58508(KMT2C)_46 | 0 | — |
| chr7 | 152158856 | 152159072 | 464_95008_58508(KMT2C)_45 | 0 | — |
| chr7 | 152162116 | 152163826 | 464_95007_58508(KMT2C)_44 | 0 | — |
| chr7 | 152167145 | 152167378 | 464_95006_58508(KMT2C)_43 | 0 | — |
| chr7 | 152169185 | 152169249 | 464_95005_58508(KMT2C)_42 | 0 | — |
| chr7 | 152171263 | 152171342 | 464_95004_58508(KMT2C)_41 | 0 | — |
| chr7 | 152174130 | 152174242 | 464_95003_58508(KMT2C)_40 | 0 | — |
| chr7 | 152176190 | 152178010 | 464_95002_58508(KMT2C)_39 | 0 | — |
| chr7 | 152179833 | 152180126 | 464_95001_58508(KMT2C)_38 | 0 | — |
| chr7 | 152180710 | 152182594 | 464_95000_58508(KMT2C)_37 | 0 | — |
| chr7 | 152182973 | 152183156 | 464_94999_58508(KMT2C)_36 | 0 | — |
| chr7 | 152185557 | 152185631 | 464_94998_58508(KMT2C)_35 | 0 | — |
| chr7 | 152187261 | 152187476 | 464_94997_58508(KMT2C)_34 | 0 | — |
| chr7 | 152187714 | 152187847 | 464_94996_58508(KMT2C)_33 | 0 | — |
| chr7 | 152194008 | 152194128 | 464_94995_58508(KMT2C)_32 | 0 | — |
| chr7 | 152194228 | 152194261 | 464_94994_58508(KMT2C)_31 | 0 | — |
| chr7 | 152194439 | 152194568 | 464_94993_58508(KMT2C)_30 | 0 | — |
| chr7 | 152195521 | 152195569 | 464_94992_58508(KMT2C)_29 | 0 | — |
| chr7 | 152195906 | 152196011 | 464_94991_58508(KMT2C)_28 | 0 | — |
| chr7 | 152199278 | 152199459 | 464_94990_58508(KMT2C)_27 | 0 | — |
| chr7 | 152202933 | 152203064 | 464_94989_58508(KMT2C)_26 | 0 | — |
| chr7 | 152205105 | 152205225 | 464_94988_58508(KMT2C)_25 | 0 | — |
| chr7 | 152207299 | 152207428 | 464_94987_58508(KMT2C)_24 | 0 | — |
| chr7 | 152220522 | 152220735 | 464_94986_58508(KMT2C)_23 | 0 | — |
| chr7 | 152222000 | 152222066 | 464_94985_58508(KMT2C)_22 | 0 | — |
| chr7 | 152222572 | 152222682 | 464_94984_58508(KMT2C)_21 | 0 | — |
| chr7 | 152224014 | 152224179 | 464_94983_58508(KMT2C)_20 | 0 | — |
| chr7 | 152224434 | 152224616 | 464_94982_58508(KMT2C)_19 | 0 | — |
| chr7 | 152229922 | 152230027 | 464_94981_58508(KMT2C)_18 | 0 | — |
| chr7 | 152230219 | 152230321 | 464_94980_58508(KMT2C)_17 | 0 | — |
| chr7 | 152235816 | 152235933 | 464_94979_58508(KMT2C)_16 | 0 | — |
| chr7 | 152238706 | 152238826 | 464_94978_58508(KMT2C)_15 | 0 | — |
| chr7 | 152247901 | 152248620 | 464_94977_58508(KMT2C)_14 | 0 | — |
| chr7 | 152249875 | 152249953 | 464_94976_58508(KMT2C)_13 | 0 | — |
| chr7 | 152250852 | 152250966 | 464_94975_58508(KMT2C)_12 | 0 | — |
| chr7 | 152251938 | 152252090 | 464_94974_58508(KMT2C)_11 | 0 | — |
| chr7 | 152252545 | 152252715 | 464_94973_58508(KMT2C)_10 | 0 | — |
| chr7 | 152263015 | 152263130 | 464_94972_58508(KMT2C)_9 | 0 | — |
| chr7 | 152265037 | 152265209 | 464_94971_58508(KMT2C)_8 | 0 | — |
| chr7 | 152273704 | 152273867 | 464_94970_58508(KMT2C)_7 | 0 | — |
| chr7 | 152309965 | 152310075 | 464_94969_58508(KMT2C)_6 | 0 | — |
| chr7 | 152311797 | 152311946 | 464_94968_58508(KMT2C)_5 | 0 | — |
| chr7 | 152315137 | 152315338 | 464_94967_58508(KMT2C)_4 | 0 | — |
| chr7 | 152330600 | 152330742 | 464_94966_58508(KMT2C)_3 | 0 | — |
| chr7 | 152358586 | 152358675 | 464_94965_58508(KMT2C)_2 | 0 | — |
| chr7 | 152435625 | 152435786 | 464_94964_58508(KMT2C)_1 | 0 | — |
| chr8 | 38413627 | 38413804 | 461_60334_2260(FGFR1)_21a | 0 | — |
| chr8 | 38413917 | 38414023 | 461_60333_2260(FGFR1)_20a | 0 | — |
| chr8 | 38414151 | 38414289 | 461_60332_2260(FGFR1)_19a | 0 | — |
| chr8 | 38414558 | 38414629 | 461_60331_2260(FGFR1)_18a | 0 | — |
| chr8 | 38414778 | 38414901 | 461_60330_2260(FGFR1)_17a | 0 | — |
| chr8 | 38415869 | 38416060 | 461_60329_2260(FGFR1)_16a | 0 | — |
| chr8 | 38417305 | 38417416 | 461_60328_2260(FGFR1)_15a | 0 | — |
| chr8 | 38417869 | 38417991 | 461_60327_2260(FGFR1)_14a | 0 | — |
| chr8 | 38418227 | 38418373 | 461_60326_2260(FGFR1)_13a | 0 | — |
| chr8 | 38419532 | 38419735 | 461_60325_2260(FGFR1)_12a | 0 | — |
| chr8 | 38421796 | 38421941 | 461_60324_2260(FGFR1)_11a | 0 | — |
| chr8 | 38424508 | 38424699 | 461_60323_2260(FGFR1)_10a | 0 | — |
| chr8 | 38426121 | 38426245 | 461_60322_2260(FGFR1)_9a | 0 | — |
| chr8 | 38427920 | 38428093 | 461_60321_2260(FGFR1)_8a | 0 | — |
| chr8 | 38428345 | 38428435 | 461_60320_2260(FGFR1)_7a | 0 | — |
| chr8 | 38429681 | 38429948 | 461_60319_2260(FGFR1)_6a | 0 | — |
| chr8 | 38440305 | 38440373 | 461_60318_2260(FGFR1)_5a | 0 | — |
| chr8 | 38457355 | 38457534 | 461_60317_2260(FGFR1)_4a | 0 | — |
| chr8 | 38461095 | 38461106 | 461_60316_2260(FGFR1)_3a | 0 | — |
| chr9 | 5021987 | 5022213 | 463_81562_3717(JAK2)_3 | 0 | — |
| chr9 | 5029782 | 5029906 | 463_81563_3717(JAK2)_4 | 0 | — |
| chr9 | 5044402 | 5044520 | 463_81564_3717(JAK2)_5 | 0 | — |
| chr9 | 5050685 | 5050831 | 463_81648_3717(JAK2)_6 | 0 | — |
| chr9 | 5054562 | 5054884 | 463_81649_3717(JAK2)_7 | 0 | — |
| chr9 | 5055668 | 5055788 | 463_81650_3717(JAK2)_8 | 0 | — |
| chr9 | 5064882 | 5065040 | 463_81651_3717(JAK2)_9 | 0 | — |
| chr9 | 5066677 | 5066789 | 463_81652_3717(JAK2)_10 | 0 | — |
| chr9 | 5069021 | 5069208 | 463_81653_3717(JAK2)_11 | 0 | — |
| chr9 | 5069924 | 5070052 | 463_81654_3717(JAK2)_12 | 0 | — |
| chr9 | 5072491 | 5072626 | 463_81655_3717(JAK2)_13 | 0 | — |
| chr9 | 5073697 | 5073785 | 463_81656_3717(JAK2)_14 | 0 | — |
| chr9 | 5077452 | 5077580 | 463_81657_3717(JAK2)_15 | 0 | — |
| chr9 | 5078305 | 5078444 | 463_81658_3717(JAK2)_16 | 0 | — |
| chr9 | 5080228 | 5080380 | 463_81659_3717(JAK2)_17 | 0 | — |
| chr9 | 5080532 | 5080683 | 463_81660_3717(JAK2)_18 | 0 | — |
| chr9 | 5081724 | 5081861 | 463_81661_3717(JAK2)_19 | 0 | — |
| chr9 | 5089673 | 5089863 | 463_81662_3717(JAK2)_20 | 0 | — |
| chr9 | 5090445 | 5090570 | 463_81663_3717(JAK2)_21 | 0 | — |
| chr9 | 5090738 | 5090911 | 463_81664_3717(JAK2)_22 | 0 | — |
| chr9 | 5123003 | 5123121 | 463_81665_3717(JAK2)_23 | 0 | — |
| chr9 | 5126332 | 5126446 | 463_81666_3717(JAK2)_24 | 0 | — |
| chr9 | 5126683 | 5126791 | 463_81667_3717(JAK2)_25 | 0 | — |
| chr9 | 21968228 | 21968242 | 428_25768_1029(CDKN2A)_7a | 0 | — |
| chr9 | 21968469 | 21968771 | 428_25767_1029(CDKN2A)_6a | 0 | — |
| chr9 | 21970901 | 21971208 | 428_25766_1029(CDKN2A)_5a | 0 | — |
| chr9 | 21971328 | 21971400 | 428_25765_1029(CDKN2A)_4a | 0 | — |
| chr9 | 21974403 | 21975133 | 428_25764_1029(CDKN2A)_3a | 0 | — |
| chr9 | 21991594 | 21992480 | 428_25763_1029(CDKN2A)_2a | 0 | — |
| chr9 | 21994138 | 21994454 | 428_25762_1029(CDKN2A)_1a | 0 | — |
| chr9 | 77721322 | 77721513 | 462_70181_2776(GNAQ)_7 | 0 | — |
| chr9 | 77728513 | 77728667 | 462_70180_2776(GNAQ)_6 | 0 | — |
| chr9 | 77794462 | 77794592 | 462_70179_2776(GNAQ)_5 | 0 | — |
| chr9 | 77797519 | 77797648 | 462_70178_2776(GNAQ)_4 | 0 | — |
| chr9 | 77815615 | 77815770 | 462_70177_2776(GNAQ)_3 | 0 | — |
| chr9 | 77922160 | 77922345 | 462_70176_2776(GNAQ)_2 | 0 | — |
| chr9 | 78031099 | 78031235 | 462_70175_2776(GNAQ)_1 | 0 | — |
| chr9 | 125236591 | 125237154 | 463_79198_3309(HSPA5)_8 | 0 | — |
| chr9 | 125238140 | 125238308 | 463_79197_3309(HSPA5)_7 | 0 | — |
| chr9 | 125238589 | 125238827 | 463_79196_3309(HSPA5)_6 | 0 | — |
| chr9 | 125238940 | 125239331 | 463_79195_3309(HSPA5)_5 | 0 | — |
| chr9 | 125239420 | 125239533 | 463_79194_3309(HSPA5)_4 | 0 | — |
| chr9 | 125240171 | 125240309 | 463_79193_3309(HSPA5)_3 | 0 | — |
| chr9 | 125240675 | 125240907 | 463_79192_3309(HSPA5)_2 | 0 | — |
| chr9 | 125241004 | 125241126 | 463_79191_3309(HSPA5)_1 | 0 | — |
| chr9 | 130714319 | 130714455 | 426_351_25(ABL1)_1 | 0 | — |
| chr9 | 130835443 | 130835525 | 426_352_25(ABL1)_2 | 0 | — |
| chr9 | 130854063 | 130854237 | 426_353_25(ABL1)_3 | 0 | — |
| chr9 | 130854800 | 130855096 | 426_354_25(ABL1)_4 | 0 | — |
| chr9 | 130862762 | 130863035 | 426_355_25(ABL1)_5 | 0 | — |
| chr9 | 130872128 | 130872213 | 426_356_25(ABL1)_6 | 0 | — |
| chr9 | 130872859 | 130873037 | 426_357_25(ABL1)_7 | 0 | — |
| chr9 | 130874867 | 130875052 | 426_358_25(ABL1)_8 | 0 | — |
| chr9 | 130878414 | 130878567 | 426_359_25(ABL1)_9 | 0 | — |
| chr9 | 130880067 | 130880157 | 426_360_25(ABL1)_10 | 0 | — |
| chr9 | 130880499 | 130880664 | 426_361_25(ABL1)_11 | 0 | — |
| chr9 | 130883968 | 130885683 | 426_362_25(ABL1)_12 | 0 | — |
| chr9 | 132896234 | 132896754 | 476_182337_7248(TSC1)_24 | 0 | — |
| chr9 | 132897183 | 132897345 | 476_182336_7248(TSC1)_23 | 0 | — |
| chr9 | 132897422 | 132897610 | 476_182335_7248(TSC1)_22 | 0 | — |
| chr9 | 132900714 | 132900837 | 476_182334_7248(TSC1)_21 | 0 | — |
| chr9 | 132901588 | 132901699 | 476_182333_7248(TSC1)_20 | 0 | — |
| chr9 | 132902604 | 132902787 | 476_182332_7248(TSC1)_19 | 0 | — |
| chr9 | 132903650 | 132903817 | 476_182331_7248(TSC1)_18 | 0 | — |
| chr9 | 132904410 | 132904454 | 476_182330_7248(TSC1)_17 | 0 | — |
| chr9 | 132905580 | 132906139 | 476_182329_7248(TSC1)_16 | 0 | — |
| chr9 | 132906730 | 132906835 | 476_182328_7248(TSC1)_15 | 0 | — |
| chr9 | 132907300 | 132907370 | 476_182327_7248(TSC1)_14 | 0 | — |
| chr9 | 132910570 | 132910692 | 476_182326_7248(TSC1)_13 | 0 | — |
| chr9 | 132911001 | 132911113 | 476_182325_7248(TSC1)_12 | 0 | — |
| chr9 | 132911452 | 132911568 | 476_182324_7248(TSC1)_11 | 0 | — |
| chr9 | 132912281 | 132912457 | 476_182323_7248(TSC1)_10 | 0 | — |
| chr9 | 132921362 | 132921436 | 476_182322_7248(TSC1)_9 | 0 | — |
| chr9 | 132921818 | 132921973 | 476_182321_7248(TSC1)_8 | 0 | — |
| chr9 | 132923347 | 132923492 | 476_182320_7248(TSC1)_7 | 0 | — |
| chr9 | 132925586 | 132925739 | 476_182319_7248(TSC1)_6 | 0 | — |
| chr9 | 132927200 | 132927304 | 476_182318_7248(TSC1)_5 | 0 | — |
| chr9 | 132928766 | 132928946 | 476_182317_7248(TSC1)_4 | 0 | — |
| chr9 | 136496070 | 136497558 | 469_119168_4851(NOTCH1)_34a | 0 | — |
| chr9 | 136498898 | 136498996 | 469_119167_4851(NOTCH1)_33a | 0 | — |
| chr9 | 136499111 | 136499259 | 469_119166_4851(NOTCH1)_32a | 0 | — |
| chr9 | 136500551 | 136500847 | 469_119165_4851(NOTCH1)_31a | 0 | — |
| chr9 | 136501747 | 136501913 | 469_119164_4851(NOTCH1)_30a | 0 | — |
| chr9 | 136502000 | 136502088 | 469_119163_4851(NOTCH1)_29a | 0 | — |
| chr9 | 136502271 | 136502488 | 469_119162_4851(NOTCH1)_28a | 0 | — |
| chr9 | 136503181 | 136503330 | 469_119161_4851(NOTCH1)_27a | 0 | — |
| chr9 | 136504672 | 136505104 | 469_119160_4851(NOTCH1)_26a | 0 | — |
| chr9 | 136505309 | 136505881 | 469_119159_4851(NOTCH1)_25a | 0 | — |
| chr9 | 136506526 | 136506639 | 469_119158_4851(NOTCH1)_24a | 0 | — |
| chr9 | 136506715 | 136506973 | 469_119157_4851(NOTCH1)_23a | 0 | — |
| chr9 | 136507304 | 136507437 | 469_119156_4851(NOTCH1)_22a | 0 | — |
| chr9 | 136507954 | 136508139 | 469_119155_4851(NOTCH1)_21a | 0 | — |
| chr9 | 136508231 | 136508385 | 469_119154_4851(NOTCH1)_20a | 0 | — |
| chr9 | 136508869 | 136509071 | 469_119153_4851(NOTCH1)_19a | 0 | — |
| chr9 | 136509732 | 136509961 | 469_119152_4851(NOTCH1)_18a | 0 | — |
| chr9 | 136510652 | 136510805 | 469_119151_4851(NOTCH1)_17a | 0 | — |
| chr9 | 136511151 | 136511271 | 469_119150_4851(NOTCH1)_16a | 0 | — |
| chr9 | 136513020 | 136513134 | 469_119149_4851(NOTCH1)_15a | 0 | — |
| chr9 | 136513391 | 136513537 | 469_119148_4851(NOTCH1)_14a | 0 | — |
| chr9 | 136514509 | 136514702 | 469_119147_4851(NOTCH1)_13a | 0 | — |
| chr9 | 136515289 | 136515400 | 469_119146_4851(NOTCH1)_12a | 0 | — |
| chr9 | 136515482 | 136515716 | 469_119145_4851(NOTCH1)_11a | 0 | — |
| chr9 | 136515980 | 136516094 | 469_119144_4851(NOTCH1)_10a | 0 | — |
| chr9 | 136517271 | 136517385 | 469_119143_4851(NOTCH1)_9a | 0 | — |
| chr9 | 136517751 | 136517937 | 469_119142_4851(NOTCH1)_8a | 0 | — |
| chr9 | 136518136 | 136518292 | 469_119141_4851(NOTCH1)_7a | 0 | — |
| chr9 | 136518590 | 136518824 | 469_119140_4851(NOTCH1)_6a | 0 | — |
| chr9 | 136519442 | 136519565 | 469_119139_4851(NOTCH1)_5a | 0 | — |
| chr9 | 136522849 | 136523188 | 469_119138_4851(NOTCH1)_4a | 0 | — |
| chr9 | 136523716 | 136523979 | 469_119137_4851(NOTCH1)_3a | 0 | — |
| chr9 | 136544023 | 136544102 | 469_119136_4851(NOTCH1)_2a | 0 | — |
| chr9 | 136545725 | 136545786 | 469_119135_4851(NOTCH1)_1a | 0 | — |
| chrX | 31121832 | 31121930 | 431_46921_1756(DMD)_85 | 0 | — |
| chrX | 31126641 | 31126673 | 431_46920_1756(DMD)_84 | 0 | — |
| chrX | 31134101 | 31134194 | 431_46919_1756(DMD)_83 | 0 | — |
| chrX | 31146290 | 31146414 | 431_46918_1756(DMD)_82 | 0 | — |
| chrX | 31147274 | 31147518 | 431_46917_1756(DMD)_81 | 0 | — |
| chrX | 31169442 | 31169601 | 431_46916_1756(DMD)_80 | 0 | — |
| chrX | 31172347 | 31172413 | 431_46915_1756(DMD)_79 | 0 | — |
| chrX | 31173538 | 31173604 | 431_46914_1756(DMD)_78 | 0 | — |
| chrX | 31177931 | 31177970 | 431_46913_1756(DMD)_77 | 0 | — |
| chrX | 31178194 | 31178805 | 431_46912_1756(DMD)_76 | 0 | — |
| chrX | 31180369 | 31180481 | 431_46911_1756(DMD)_75 | 0 | — |
| chrX | 31182737 | 31182904 | 431_46910_1756(DMD)_74 | 0 | — |
| chrX | 31203960 | 31204118 | 431_46909_1756(DMD)_73 | 0 | — |
| chrX | 31206581 | 31206667 | 431_46908_1756(DMD)_72 | 0 | — |
| chrX | 31209497 | 31209699 | 431_46907_1756(DMD)_71 | 0 | — |
| chrX | 31223046 | 31223121 | 431_46906_1756(DMD)_70 | 0 | — |
| chrX | 31260954 | 31261016 | 431_46905_1756(DMD)_69 | 0 | — |
| chrX | 31266809 | 31266907 | 431_46904_1756(DMD)_68 | 0 | — |
| chrX | 31323597 | 31323658 | 431_46903_1756(DMD)_67 | 0 | — |
| chrX | 31348555 | 31348634 | 431_46902_1756(DMD)_66 | 0 | — |
| chrX | 31444480 | 31444627 | 431_46901_1756(DMD)_65 | 0 | — |
| chrX | 31478105 | 31478374 | 431_46900_1756(DMD)_64 | 0 | — |
| chrX | 31478982 | 31479103 | 431_46899_1756(DMD)_63 | 0 | — |
| chrX | 31496787 | 31496944 | 431_46898_1756(DMD)_62 | 0 | — |
| chrX | 31507280 | 31507453 | 431_46897_1756(DMD)_61 | 0 | — |
| chrX | 31508207 | 31508298 | 431_46896_1756(DMD)_60 | 0 | — |
| chrX | 31627672 | 31627862 | 431_46895_1756(DMD)_59 | 0 | — |
| chrX | 31657989 | 31658144 | 431_46894_1756(DMD)_58 | 0 | — |
| chrX | 31679374 | 31679586 | 431_46893_1756(DMD)_57 | 0 | — |
| chrX | 31729630 | 31729748 | 431_46892_1756(DMD)_56 | 0 | — |
| chrX | 31773959 | 31774192 | 431_46891_1756(DMD)_55 | 0 | — |
| chrX | 31819974 | 31820083 | 431_46890_1756(DMD)_54 | 0 | — |
| chrX | 31836717 | 31836819 | 431_46889_1756(DMD)_53 | 0 | — |
| chrX | 31875187 | 31875373 | 431_46888_1756(DMD)_52 | 0 | — |
| chrX | 31929595 | 31929745 | 431_46887_1756(DMD)_51 | 0 | — |
| chrX | 31932079 | 31932227 | 431_46886_1756(DMD)_50 | 0 | — |
| chrX | 31968338 | 31968514 | 431_46885_1756(DMD)_49 | 0 | — |
| chrX | 32155370 | 32155469 | 431_46884_1756(DMD)_48 | 0 | — |
| chrX | 32216915 | 32217063 | 431_46883_1756(DMD)_47 | 0 | — |
| chrX | 32287528 | 32287701 | 431_46882_1756(DMD)_46 | 0 | — |
| chrX | 32310081 | 32310276 | 431_46881_1756(DMD)_45 | 0 | — |
| chrX | 32342099 | 32342282 | 431_46880_1756(DMD)_44 | 0 | — |
| chrX | 32343133 | 32343286 | 431_46879_1756(DMD)_43 | 0 | — |
| chrX | 32345942 | 32346080 | 431_46878_1756(DMD)_42 | 0 | — |
| chrX | 32348405 | 32348528 | 431_46877_1756(DMD)_41 | 0 | — |
| chrX | 32362787 | 32362958 | 431_46876_1756(DMD)_40 | 0 | — |
| chrX | 32364581 | 32364710 | 431_46875_1756(DMD)_39 | 0 | — |
| chrX | 32365019 | 32365199 | 431_46874_1756(DMD)_38 | 0 | — |
| chrX | 32380509 | 32380680 | 431_46873_1756(DMD)_37 | 0 | — |
| chrX | 32386309 | 32386465 | 431_46872_1756(DMD)_36 | 0 | — |
| chrX | 32389500 | 32389674 | 431_46871_1756(DMD)_35 | 0 | — |
| chrX | 32390070 | 32390181 | 431_46870_1756(DMD)_34 | 0 | — |
| chrX | 32411751 | 32411913 | 431_46869_1756(DMD)_33 | 0 | — |
| chrX | 32412018 | 32412254 | 431_46868_1756(DMD)_32 | 0 | — |
| chrX | 32438240 | 32438390 | 431_46867_1756(DMD)_31 | 0 | — |
| chrX | 32441179 | 32441314 | 431_46866_1756(DMD)_30 | 0 | — |
| chrX | 32448455 | 32448638 | 431_46865_1756(DMD)_29 | 0 | — |
| chrX | 32454661 | 32454832 | 431_46864_1756(DMD)_28 | 0 | — |
| chrX | 32463438 | 32463594 | 431_46863_1756(DMD)_27 | 0 | — |
| chrX | 32464585 | 32464699 | 431_46862_1756(DMD)_26 | 0 | — |
| chrX | 32468497 | 32468710 | 431_46861_1756(DMD)_25 | 0 | — |
| chrX | 32472163 | 32472309 | 431_46860_1756(DMD)_24 | 0 | — |
| chrX | 32484918 | 32485099 | 431_46859_1756(DMD)_23 | 0 | — |
| chrX | 32491276 | 32491518 | 431_46858_1756(DMD)_22 | 0 | — |
| chrX | 32501754 | 32501842 | 431_46857_1756(DMD)_21 | 0 | — |
| chrX | 32518007 | 32518131 | 431_46856_1756(DMD)_20 | 0 | — |
| chrX | 32545158 | 32545334 | 431_46855_1756(DMD)_19 | 0 | — |
| chrX | 32565701 | 32565881 | 431_46854_1756(DMD)_18 | 0 | — |
| chrX | 32573529 | 32573637 | 431_46853_1756(DMD)_17 | 0 | — |
| chrX | 32573744 | 32573846 | 431_46852_1756(DMD)_16 | 0 | — |
| chrX | 32595756 | 32595876 | 431_46851_1756(DMD)_15 | 0 | — |
| chrX | 32614302 | 32614453 | 431_46850_1756(DMD)_14 | 0 | — |
| chrX | 32644131 | 32644313 | 431_46849_1756(DMD)_13 | 0 | — |
| chrX | 32644963 | 32645152 | 431_46848_1756(DMD)_12 | 0 | — |
| chrX | 32697869 | 32697998 | 431_46847_1756(DMD)_11 | 0 | — |
| chrX | 32699111 | 32699293 | 431_46846_1756(DMD)_10 | 0 | — |
| chrX | 32809492 | 32809611 | 431_46845_1756(DMD)_9 | 0 | — |
| chrX | 32816467 | 32816640 | 431_46844_1756(DMD)_8 | 0 | — |
| chrX | 32823294 | 32823387 | 431_46843_1756(DMD)_7 | 0 | — |
| chrX | 32844782 | 32844860 | 431_46842_1756(DMD)_6 | 0 | — |
| chrX | 32849727 | 32849820 | 431_46841_1756(DMD)_5 | 0 | — |
| chrX | 33020138 | 33020200 | 431_46840_1756(DMD)_4 | 0 | — |
| chrX | 33128062 | 33128428 | 431_46839_1756(DMD)_3 | 0 | — |
| chrX | 33211281 | 33211556 | 431_46838_1756(DMD)_2 | 0 | — |
| chrX | 33339258 | 33339265 | 431_46837_1756(DMD)_1 | 0 | — |
| chrX | 47145446 | 47145516 | 472_141747_8241(RBM10)_1 | 0 | — |
| chrX | 47147356 | 47147498 | 472_141748_8241(RBM10)_2 | 0 | — |
| chrX | 47169314 | 47169498 | 472_141749_8241(RBM10)_3 | 0 | — |
| chrX | 47171027 | 47171258 | 472_141750_8241(RBM10)_4 | 0 | — |
| chrX | 47173127 | 47173197 | 472_141751_8241(RBM10)_5 | 0 | — |
| chrX | 47175018 | 47175092 | 472_141752_8241(RBM10)_6 | 0 | — |
| chrX | 47176499 | 47176586 | 472_141753_8241(RBM10)_7 | 0 | — |
| chrX | 47179102 | 47179163 | 472_141754_8241(RBM10)_8 | 0 | — |
| chrX | 47179318 | 47179495 | 472_141755_8241(RBM10)_9 | 0 | — |
| chrX | 47179879 | 47180040 | 472_141756_8241(RBM10)_10 | 0 | — |
| chrX | 47180211 | 47180309 | 472_141757_8241(RBM10)_11 | 0 | — |
| chrX | 47180418 | 47180506 | 472_141758_8241(RBM10)_12 | 0 | — |
| chrX | 47181214 | 47181401 | 472_141759_8241(RBM10)_13 | 0 | — |
| chrX | 47181506 | 47181646 | 472_141760_8241(RBM10)_14 | 0 | — |
| chrX | 47181748 | 47181866 | 472_141761_8241(RBM10)_15 | 0 | — |
| chrX | 47181950 | 47182042 | 472_141762_8241(RBM10)_16 | 0 | — |
| chrX | 47182161 | 47182326 | 472_141763_8241(RBM10)_17 | 0 | — |
| chrX | 47185054 | 47185204 | 472_141764_8241(RBM10)_18 | 0 | — |
| chrX | 47185301 | 47185367 | 472_141765_8241(RBM10)_19 | 0 | — |
| chrX | 47185441 | 47185630 | 472_141766_8241(RBM10)_20 | 0 | — |
| chrX | 47185715 | 47185790 | 472_141767_8241(RBM10)_21 | 0 | — |
| chrX | 47186064 | 47186171 | 472_141768_8241(RBM10)_22 | 0 | — |
| chrX | 47186257 | 47186387 | 472_141769_8241(RBM10)_23 | 0 | — |
| chrX | 47186473 | 47186599 | 472_141770_8241(RBM10)_24 | 0 | — |

### Supplementary Data 2

| | | | | | |
|---|---|---|---|---|---|
| chr9 | 136496070 | 136497558 | 469_119168_4851(NOTCH1)_34a | 0 | — |
| chr9 | 136499111 | 136499259 | 469_119166_4851(NOTCH1)_32a | 0 | — |
| chr9 | 136500551 | 136500847 | 469_119165_4851(NOTCH1)_31a | 0 | — |
| chr9 | 136501747 | 136501913 | 469_119164_4851(NOTCH1)_30a | 0 | — |
| chr9 | 136502000 | 136502088 | 469_119163_4851(NOTCH1)_29a | 0 | — |
| chr9 | 136502271 | 136502488 | 469_119162_4851(NOTCH1)_28a | 0 | — |
| chr9 | 136503181 | 136503330 | 469_119161_4851(NOTCH1)_27a | 0 | — |
| chr9 | 136504672 | 136505104 | 469_119160_4851(NOTCH1)_26a | 0 | — |
| chr9 | 136506526 | 136506639 | 469_119158_4851(NOTCH1)_24a | 0 | — |
| chr9 | 136506715 | 136506973 | 469_119157_4851(NOTCH1)_23a | 0 | — |
| chr9 | 136507304 | 136507437 | 469_119156_4851(NOTCH1)_22a | 0 | — |
| chr9 | 136507954 | 136508139 | 469_119155_4851(NOTCH1)_21a | 0 | — |
| chr9 | 136508231 | 136508385 | 469_119154_4851(NOTCH1)_20a | 0 | — |
| chr9 | 136508869 | 136509071 | 469_119153_4851(NOTCH1)_19a | 0 | — |
| chr9 | 136509732 | 136509961 | 469_119152_4851(NOTCH1)_18a | 0 | — |
| chr9 | 136511151 | 136511271 | 469_119150_4851(NOTCH1)_16a | 0 | — |
| chr9 | 136513020 | 136513134 | 469_119149_4851(NOTCH1)_15a | 0 | — |
| chr9 | 136513391 | 136513537 | 469_119148_4851(NOTCH1)_14a | 0 | — |
| chr9 | 136514509 | 136514702 | 469_119147_4851(NOTCH1)_13a | 0 | — |
| chr9 | 136515289 | 136515400 | 469_119146_4851(NOTCH1)_12a | 0 | — |
| chr9 | 136515482 | 136515716 | 469_119145_4851(NOTCH1)_11a | 0 | — |
| chr9 | 136515980 | 136516094 | 469_119144_4851(NOTCH1)_10a | 0 | — |
| chr9 | 136517751 | 136517937 | 469_119142_4851(NOTCH1)_8a | 0 | — |
| chr9 | 136518136 | 136518292 | 469_119141_4851(NOTCH1)_7a | 0 | — |
| chr9 | 136518590 | 136518824 | 469_119140_4851(NOTCH1)_6a | 0 | — |
| chr9 | 136519442 | 136519565 | 469_119139_4851(NOTCH1)_5a | 0 | — |
| chr9 | 136522849 | 136523188 | 469_119138_4851(NOTCH1)_4a | 0 | — |
| chr9 | 136523716 | 136523979 | 469_119137_4851(NOTCH1)_3a | 0 | — |
| chr9 | 136544023 | 136544102 | 469_119136_4851(NOTCH1)_2a | 0 | — |
| chr6 | 32195436 | 32196421 | 469_119235_4855(NOTCH4)_29 | 0 | — |
| chr6 | 32197298 | 32197594 | 469_119233_4855(NOTCH4)_27 | 0 | — |
| chr6 | 32198420 | 32198559 | 469_119232_4855(NOTCH4)_26 | 0 | — |
| chr6 | 32198648 | 32198730 | 469_119231_4855(NOTCH4)_25 | 0 | — |
| chr6 | 32198925 | 32199145 | 469_119230_4855(NOTCH4)_24 | 0 | — |
| chr6 | 32200830 | 32201006 | 469_119229_4855(NOTCH4)_23 | 0 | — |
| chr6 | 32201116 | 32201500 | 469_119228_4855(NOTCH4)_22 | 0 | — |
| chr6 | 32202075 | 32202599 | 469_119227_4855(NOTCH4)_21 | 0 | — |
| chr6 | 32204136 | 32204389 | 469_119225_4855(NOTCH4)_19 | 0 | — |
| chr6 | 32210751 | 32210936 | 469_119224_4855(NOTCH4)_18 | 0 | — |
| chr6 | 32212473 | 32212627 | 469_119223_4855(NOTCH4)_17 | 0 | — |
| chr6 | 32212823 | 32212911 | 469_119222_4855(NOTCH4)_16 | 0 | — |
| chr6 | 32213134 | 32213252 | 469_119221_4855(NOTCH4)_15 | 0 | — |
| chr6 | 32213687 | 32213840 | 469_119220_4855(NOTCH4)_14 | 0 | — |
| chr6 | 32214109 | 32214255 | 469_119219_4855(NOTCH4)_13 | 0 | — |
| chr6 | 32216944 | 32217067 | 469_119217_4855(NOTCH4)_11 | 0 | — |
| chr6 | 32217152 | 32217266 | 469_119216_4855(NOTCH4)_10 | 0 | — |
| chr6 | 32217994 | 32218108 | 469_119215_4855(NOTCH4)_9 | 0 | — |
| chr6 | 32219591 | 32219786 | 469_119214_4855(NOTCH4)_8 | 0 | — |
| chr6 | 32220128 | 32220284 | 469_119213_4855(NOTCH4)_7 | 0 | — |
| chr6 | 32220404 | 32220641 | 469_119212_4855(NOTCH4)_6 | 0 | — |
| chr6 | 32220755 | 32220878 | 469_119211_4855(NOTCH4)_5 | 0 | — |
| chr6 | 32222510 | 32222806 | 469_119209_4855(NOTCH4)_3 | 0 | — |
| chr6 | 32223004 | 32223086 | 469_119208_4855(NOTCH4)_2 | 0 | — |
| chr6 | 32223855 | 32223928 | 469_119207_4855(NOTCH4)_1 | 0 | — |
| chr5 | 171410526 | 171410565 | 469_119426_4869(NPM1)_12 | 0 | — |
| chr5 | 171407699 | 171407774 | 469_119425_4869(NPM1)_11 | 0 | — |
| chr5 | 171406396 | 171406734 | 469_119424_4869(NPM1)_10 | 0 | — |
| chr5 | 171405301 | 171405403 | 469_119423_4869(NPM1)_9 | 0 | — |
| chr5 | 171400838 | 171400925 | 469_119422_4869(NPM1)_8 | 0 | — |
| chr5 | 171392913 | 171392978 | 469_119420_4869(NPM1)_6 | 0 | — |
| chr5 | 171392709 | 171392816 | 469_119419_4869(NPM1)_5 | 0 | — |
| chr5 | 171391705 | 171391799 | 469_119418_4869(NPM1)_4 | 0 | — |
| chr5 | 171391304 | 171391424 | 469_119417_4869(NPM1)_3 | 0 | — |
| chr5 | 171390050 | 171390130 | 469_119416_4869(NPM1)_2 | 0 | — |
| chr5 | 171387948 | 171388006 | 469_119415_4869(NPM1)_1 | 0 | — |
| chr1 | 114708534 | 114708654 | 469_119576_4893(NRAS)_5 | 0 | — |
| chr1 | 114709568 | 114709728 | 469_119575_4893(NRAS)_4 | 0 | — |
| chr1 | 114713799 | 114713978 | 469_119574_4893(NRAS)_3 | 0 | — |
| chr1 | 114716049 | 114716160 | 469_119573_4893(NRAS)_2 | 0 | — |
| chr17 | 7669608 | 7669690 | 476_181712_7157(TP53)_11 | 0 | — |
| chr17 | 7670608 | 7670715 | 476_181711_7157(TP53)_10 | 0 | — |
| chr17 | 7673206 | 7673339 | 476_181710_7157(TP53)_9 | 0 | — |
| chr17 | 7673700 | 7673837 | 476_181708_7157(TP53)_7 | 0 | — |
| chr17 | 7674180 | 7674290 | 476_181707_7157(TP53)_6 | 0 | — |
| chr17 | 7674858 | 7674971 | 476_181706_7157(TP53)_5 | 0 | — |
| chr17 | 7675052 | 7675493 | 476_181705_7157(TP53)_4 | 0 | — |
| chr17 | 7675993 | 7676272 | 476_181704_7157(TP53)_3 | 0 | — |
| chr17 | 7676381 | 7676594 | 476_181703_7157(TP53)_2 | 0 | — |
| chr17 | 31349119 | 31349251 | 469_118622_4763(NF1)_49a | 0 | — |
| chr17 | 31343008 | 31343135 | 469_118621_4763(NF1)_48a | 0 | — |
| chr17 | 31340504 | 31340645 | 469_118620_4763(NF1)_47a | 0 | — |
| chr17 | 31338024 | 31338139 | 469_118618_4763(NF1)_45a | 0 | — |
| chr17 | 31337818 | 31337880 | 469_118617_4763(NF1)_44a | 0 | — |
| chr17 | 31337367 | 31337582 | 469_118616_4763(NF1)_43a | 0 | — |
| chr17 | 31336634 | 31336914 | 469_118615_4763(NF1)_42a | 0 | — |
| chr17 | 31336332 | 31336473 | 469_118614_4763(NF1)_41a | 0 | — |
| chr17 | 31334837 | 31335031 | 469_118613_4763(NF1)_40a | 0 | — |
| chr17 | 31327498 | 31327839 | 469_118611_4763(NF1)_38a | 0 | — |
| chr17 | 31325819 | 31326252 | 469_118610_4763(NF1)_37a | 0 | — |
| chr17 | 31265228 | 31265339 | 469_118609_4763(NF1)_36a | 0 | — |
| chr17 | 31261710 | 31261857 | 469_118608_4763(NF1)_35a | 0 | — |
| chr17 | 31260368 | 31260515 | 469_118607_4763(NF1)_34a | 0 | — |
| chr17 | 31259031 | 31259129 | 469_118606_4763(NF1)_33a | 0 | — |
| chr17 | 31258343 | 31258502 | 469_118605_4763(NF1)_32a | 0 | — |
| chr17 | 31248983 | 31249119 | 469_118603_4763(NF1)_30a | 0 | — |
| chr17 | 31235917 | 31236021 | 469_118602_4763(NF1)_29a | 0 | — |
| chr17 | 31235610 | 31235772 | 469_118601_4763(NF1)_28a | 0 | — |
| chr17 | 31233001 | 31233213 | 469_118600_4763(NF1)_27a | 0 | — |
| chr17 | 31232699 | 31232881 | 469_118599_4763(NF1)_26a | 0 | — |
| chr17 | 31232072 | 31232189 | 469_118598_4763(NF1)_25a | 0 | — |
| chr17 | 31230259 | 31230382 | 469_118596_4763(NF1)_23a | 0 | — |
| chr17 | 31229834 | 31229974 | 469_118595_4763(NF1)_22a | 0 | — |
| chr17 | 31229024 | 31229465 | 469_118594_4763(NF1)_21a | 0 | — |
| chr17 | 31227522 | 31227606 | 469_118593_4763(NF1)_20a | 0 | — |
| chr17 | 31227217 | 31227291 | 469_118592_4763(NF1)_19a | 0 | — |
| chr17 | 31226434 | 31226684 | 469_118591_4763(NF1)_18a | 0 | — |
| chr17 | 31223443 | 31223567 | 469_118589_4763(NF1)_16a | 0 | — |
| chr17 | 31221849 | 31222764 | 469_118588_4763(NF1)_15a | 0 | — |
| chr17 | 31219004 | 31219118 | 469_118587_4763(NF1)_14a | 0 | — |
| chr17 | 31214450 | 31214585 | 469_118586_4763(NF1)_13a | 0 | — |
| chr17 | 31206239 | 31206371 | 469_118585_4763(NF1)_12a | 0 | — |
| chr17 | 31201410 | 31201485 | 469_118584_4763(NF1)_11a | 0 | — |
| chr17 | 31200421 | 31200595 | 469_118582_4763(NF1)_9a | 0 | — |
| chr17 | 31182507 | 31182665 | 469_118581_4763(NF1)_8a | 0 | — |
| chr17 | 31181709 | 31181785 | 469_118580_4763(NF1)_7a | 0 | — |
| chr17 | 31181421 | 31181489 | 469_118579_4763(NF1)_6a | 0 | — |
| chr17 | 31169890 | 31169997 | 469_118578_4763(NF1)_5a | 0 | — |
| chr17 | 31163185 | 31163376 | 469_118577_4763(NF1)_4a | 0 | — |
| chr17 | 31155982 | 31156126 | 469_118575_4763(NF1)_2a | 0 | — |
| chr17 | 31095309 | 31095369 | 469_118574_4763(NF1)_1a | 0 | — |
| chr17 | 31367224 | 31367278 | 469_118632_4763(NF1)_59a | 0 | — |
| chr17 | 31362287 | 31362364 | 469_118631_4763(NF1)_58a | 0 | — |
| chr17 | 31360486 | 31360703 | 469_118630_4763(NF1)_57a | 0 | — |
| chr17 | 31358968 | 31359015 | 469_118629_4763(NF1)_56a | 0 | — |
| chr17 | 31358479 | 31358622 | 469_118628_4763(NF1)_55a | 0 | — |
| chr17 | 31357268 | 31357369 | 469_118627_4763(NF1)_54a | 0 | — |
| chr9 | 136498898 | 136498996 | 469_119167_4851(NOTCH1)_33a | 0 | — |
| chr9 | 136505309 | 136505881 | 469_119159_4851(NOTCH1)_25a | 0 | — |
| chr9 | 136510652 | 136510805 | 469_119151_4851(NOTCH1)_17a | 0 | — |
| chr9 | 136517271 | 136517385 | 469_119143_4851(NOTCH1)_9a | 0 | — |
| chr9 | 136545725 | 136545786 | 469_119135_4851(NOTCH1)_1a | 0 | — |
| chr6 | 32196924 | 32197072 | 469_119234_4855(NOTCH4)_28 | 0 | — |
| chr6 | 32203769 | 32203882 | 469_119226_4855(NOTCH4)_20 | 0 | — |
| chr6 | 32215225 | 32215385 | 469_119218_4855(NOTCH4)_12 | 0 | — |
| chr6 | 32220977 | 32221325 | 469_119210_4855(NOTCH4)_4 | 0 | — |
| chr3 | 179234093 | 179234364 | 470_127823_5290(PIK3CA)_21 | 0 | — |
| chr3 | 179230003 | 179230121 | 470_127821_5290(PIK3CA)_19 | 0 | — |
| chr3 | 179229271 | 179229442 | 470_127820_5290(PIK3CA)_18 | 0 | — |
| chr3 | 179225961 | 179226040 | 470_127819_5290(PIK3CA)_17 | 0 | — |
| chr3 | 179224699 | 179224821 | 470_127818_5290(PIK3CA)_16 | 0 | — |
| chr3 | 179224080 | 179224187 | 470_127817_5290(PIK3CA)_15 | 0 | — |
| chr3 | 179220985 | 179221157 | 470_127816_5290(PIK3CA)_14 | 0 | — |
| chr3 | 179219570 | 179219735 | 470_127814_5290(PIK3CA)_12 | 0 | — |
| chr3 | 179219195 | 179219277 | 470_127813_5290(PIK3CA)_11 | 0 | — |
| chr3 | 179218209 | 179218334 | 470_127812_5290(PIK3CA)_10 | 0 | — |
| chr3 | 179210430 | 179210565 | 470_127811_5290(PIK3CA)_9 | 0 | — |
| chr3 | 179210185 | 179210338 | 470_127810_5290(PIK3CA)_8 | 0 | — |
| chr3 | 179209594 | 179209700 | 470_127809_5290(PIK3CA)_7 | 0 | — |
| chr3 | 179204502 | 179204588 | 470_127808_5290(PIK3CA)_6 | 0 | — |
| chr3 | 179201289 | 179201540 | 470_127806_5290(PIK3CA)_4 | 0 | — |
| chr3 | 179199689 | 179199899 | 470_127805_5290(PIK3CA)_3 | 0 | — |
| chr3 | 179198825 | 179199177 | 470_127804_5290(PIK3CA)_2 | 0 | — |
| chr3 | 179230224 | 179230376 | 470_127822_5290(PIK3CA)_20 | 0 | — |
| chr3 | 179219948 | 179220052 | 470_127815_5290(PIK3CA)_13 | 0 | — |
| chr3 | 179203543 | 179203789 | 470_127807_5290(PIK3CA)_5 | 0 | — |
| chr5 | 68297411 | 68297601 | 470_127791_5295(PIK3R1)_19 | 0 | — |
| chr5 | 68296170 | 68296341 | 470_127790_5295(PIK3R1)_18 | 0 | — |
| chr5 | 68295419 | 68295488 | 470_127789_5295(PIK3R1)_17 | 0 | — |
| chr5 | 68295147 | 68295324 | 470_127788_5295(PIK3R1)_16 | 0 | — |
| chr5 | 68293708 | 68293834 | 470_127786_5295(PIK3R1)_14 | 0 | — |
| chr5 | 68293302 | 68293483 | 470_127785_5295(PIK3R1)_13 | 0 | — |
| chr5 | 68293100 | 68293199 | 470_127784_5295(PIK3R1)_12 | 0 | — |
| chr5 | 68292567 | 68292729 | 470_127783_5295(PIK3R1)_11 | 0 | — |
| chr5 | 68292258 | 68292361 | 470_127782_5295(PIK3R1)_10 | 0 | — |
| chr5 | 68290638 | 68290834 | 470_127781_5295(PIK3R1)_9 | 0 | — |
| chr5 | 68280926 | 68281006 | 470_127779_5295(PIK3R1)_7 | 0 | — |
| chr5 | 68280527 | 68280729 | 470_127778_5295(PIK3R1)_6 | 0 | — |
| chr5 | 68279601 | 68279733 | 470_127777_5295(PIK3R1)_5 | 0 | — |
| chr5 | 68273938 | 68274013 | 470_127776_5295(PIK3R1)_4 | 0 | — |
| chr5 | 68273389 | 68273482 | 470_127775_5295(PIK3R1)_3 | 0 | — |
| chr5 | 68226675 | 68227009 | 470_127774_5295(PIK3R1)_2 | 0 | — |
| chr5 | 68294535 | 68294678 | 470_127787_5295(PIK3R1)_15 | 0 | — |
| chr5 | 68288423 | 68288751 | 470_127780_5295(PIK3R1)_8 | 0 | — |
| chr10 | 43124882 | 43124982 | 473_148936_5979(RET)_18a | 0 | — |
| chr10 | 43114479 | 43114736 | 473_148929_5979(RET)_11a | 0 | — |
| chr10 | 43104951 | 43105193 | 473_148922_5979(RET)_4a | 0 | — |
| chr17 | 58363525 | 58363600 | 473_154856_54894(RNF43)_4 | 0 | — |
| chr17 | 7673534 | 7673608 | 476_181709_7157(TP53)_8 | 0 | — |
| chr9 | 132896234 | 132896754 | 476_182337_7248(TSC1)_24 | 0 | — |
| chr9 | 132897183 | 132897345 | 476_182336_7248(TSC1)_23 | 0 | — |
| chr9 | 132897422 | 132897610 | 476_182335_7248(TSC1)_22 | 0 | — |
| chr9 | 132900714 | 132900837 | 476_182334_7248(TSC1)_21 | 0 | — |
| chr9 | 132901588 | 132901699 | 476_182333_7248(TSC1)_20 | 0 | — |
| chr9 | 132902604 | 132902787 | 476_182332_7248(TSC1)_19 | 0 | — |
| chr9 | 132903650 | 132903817 | 476_182331_7248(TSC1)_18 | 0 | — |
| chr9 | 132905580 | 132906139 | 476_182329_7248(TSC1)_16 | 0 | — |
| chr9 | 132906730 | 132906835 | 476_182328_7248(TSC1)_15 | 0 | — |
| chr9 | 132907300 | 132907370 | 476_182327_7248(TSC1)_14 | 0 | — |
| chr9 | 132910570 | 132910692 | 476_182326_7248(TSC1)_13 | 0 | — |
| chr9 | 132911001 | 132911113 | 476_182325_7248(TSC1)_12 | 0 | — |
| chr9 | 132911452 | 132911568 | 476_182324_7248(TSC1)_11 | 0 | — |
| chr9 | 132912281 | 132912457 | 476_182323_7248(TSC1)_10 | 0 | — |
| chr9 | 132921818 | 132921973 | 476_182321_7248(TSC1)_8 | 0 | — |
| chr9 | 132923347 | 132923492 | 476_182320_7248(TSC1)_7 | 0 | — |
| chr9 | 132925586 | 132925739 | 476_182319_7248(TSC1)_6 | 0 | — |
| chr9 | 132927200 | 132927304 | 476_182318_7248(TSC1)_5 | 0 | — |
| chr9 | 132928766 | 132928946 | 476_182317_7248(TSC1)_4 | 0 | — |
| chr9 | 132921362 | 132921436 | 476_182322_7248(TSC1)_9 | 0 | — |
| chr9 | 132904410 | 132904454 | 476_182330_7248(TSC1)_17 | 0 | — |
| chr16 | 2058746 | 2058873 | 476_182347_7249(TSC2)_10 | 0 | — |
| chr16 | 2057104 | 2057178 | 476_182346_7249(TSC2)_9 | 0 | — |
| chr16 | 2056195 | 2056244 | 476_182344_7249(TSC2)_7 | 0 | — |
| chr16 | 2055401 | 2055519 | 476_182343_7249(TSC2)_6 | 0 | — |
| chr16 | 2054295 | 2054440 | 476_182342_7249(TSC2)_5 | 0 | — |
| chr16 | 2053341 | 2053452 | 476_182341_7249(TSC2)_4 | 0 | — |
| chr16 | 2050399 | 2050486 | 476_182340_7249(TSC2)_3 | 0 | — |
| chr16 | 2048586 | 2048753 | 476_182339_7249(TSC2)_2 | 0 | — |
| chr16 | 2086192 | 2086379 | 476_182374_7249(TSC2)_37 | 0 | — |
| chr16 | 2080164 | 2080377 | 476_182367_7249(TSC2)_30 | 0 | — |
| chr16 | 2075798 | 2075892 | 476_182360_7249(TSC2)_23 | 0 | — |
| chr16 | 2065518 | 2065635 | 476_182353_7249(TSC2)_16 | 0 | — |
| chr16 | 2056643 | 2056769 | 476_182345_7249(TSC2)_8 | 0 | — |
| chr16 | 2047821 | 2048065 | 476_182338_7249(TSC2)_1 | 0 | — |
| chr3 | 37050485 | 37050653 | 466_106476_4292(MLH1)_21 | 0 | — |
| chr3 | 37048516 | 37048609 | 466_106474_4292(MLH1)_19 | 0 | — |
| chr3 | 37047518 | 37047683 | 466_106473_4292(MLH1)_18 | 0 | — |
| chr3 | 37042267 | 37042331 | 466_106472_4292(MLH1)_17 | 0 | — |
| chr3 | 37040185 | 37040294 | 466_106471_4292(MLH1)_16 | 0 | — |
| chr3 | 37028783 | 37028932 | 466_106470_4292(MLH1)_15 | 0 | — |
| chr3 | 37025636 | 37026007 | 466_106469_4292(MLH1)_14 | 0 | — |
| chr3 | 37020309 | 37020463 | 466_106468_4292(MLH1)_13 | 0 | — |
| chr3 | 37014431 | 37014544 | 466_106466_4292(MLH1)_11 | 0 | — |
| chr3 | 37012010 | 37012099 | 466_106465_4292(MLH1)_10 | 0 | — |
| chr3 | 37011819 | 37011862 | 466_106464_4292(MLH1)_9 | 0 | — |
| chr17 | 31374012 | 31374155 | 469_118633_4763(NF1)_60a | 0 | — |
| chr17 | 31356959 | 31357090 | 469_118626_4763(NF1)_53a | 0 | — |
| chr17 | 31338703 | 31338805 | 469_118619_4763(NF1)_46a | 0 | — |
| chr17 | 31330295 | 31330498 | 469_118612_4763(NF1)_39a | 0 | — |
| chr17 | 31252937 | 31253000 | 469_118604_4763(NF1)_31a | 0 | — |
| chr17 | 31230841 | 31230925 | 469_118597_4763(NF1)_24a | 0 | — |
| chr17 | 31225094 | 31225250 | 469_118590_4763(NF1)_17a | 0 | — |
| chr17 | 31201036 | 31201159 | 469_118583_4763(NF1)_10a | 0 | — |
| chr17 | 31159009 | 31159093 | 469_118576_4763(NF1)_3a | 0 | — |
| chr7 | 116695756 | 116695799 | 466_106278_4233(MET)_2a | 0 | — |
| chr7 | 116699070 | 116700284 | 466_106279_4233(MET)_3a | 0 | — |
| chr7 | 116724099 | 116724164 | 466_106280_4233(MET)_4a | 0 | — |
| chr7 | 116724771 | 116724847 | 466_106281_4233(MET)_5a | 0 | — |
| chr7 | 116731667 | 116731859 | 466_106282_4233(MET)_6a | 0 | — |
| chr7 | 116739949 | 116740084 | 466_106283_4233(MET)_7a | 0 | — |
| chr7 | 116755354 | 116755515 | 466_106285_4233(MET)_9a | 0 | — |
| chr7 | 116757436 | 116757539 | 466_106286_4233(MET)_10a | 0 | — |
| chr7 | 116757637 | 116757774 | 466_106287_4233(MET)_11a | 0 | — |
| chr7 | 116758458 | 116758620 | 466_106288_4233(MET)_12a | 0 | — |
| chr7 | 116759336 | 116759490 | 466_106289_4233(MET)_13a | 0 | — |
| chr7 | 116763049 | 116763268 | 466_106290_4233(MET)_14a | 0 | — |
| chr7 | 116771497 | 116771654 | 466_106292_4233(MET)_16a | 0 | — |
| chr7 | 116771848 | 116771989 | 466_106293_4233(MET)_17a | 0 | — |
| chr7 | 116774880 | 116775111 | 466_106294_4233(MET)_18a | 0 | — |
| chr7 | 116777388 | 116777469 | 466_106295_4233(MET)_19a | 0 | — |
| chr7 | 116778775 | 116778957 | 466_106296_4233(MET)_20a | 0 | — |
| chr7 | 116781987 | 116782097 | 466_106297_4233(MET)_21a | 0 | — |
| chr7 | 116783303 | 116783469 | 466_106298_4233(MET)_22a | 0 | — |
| chr7 | 116795886 | 116796124 | 466_106300_4233(MET)_24a | 0 | — |
| chr1 | 204525327 | 204525346 | 466_106137_4194(MDM4)_2a | 0 | — |
| chr1 | 204542783 | 204542944 | 466_106144_4194(MDM4)_9a | 0 | — |
| chr1 | 204525483 | 204525596 | 466_106138_4194(MDM4)_3a | 0 | — |
| chr1 | 204526359 | 204526434 | 466_106139_4194(MDM4)_4a | 0 | — |
| chr1 | 204530683 | 204530817 | 466_106140_4194(MDM4)_5a | 0 | — |
| chr1 | 204532190 | 204532246 | 466_106141_4194(MDM4)_6a | 0 | — |
| chr1 | 204537429 | 204537497 | 466_106142_4194(MDM4)_7a | 0 | — |
| chr1 | 204538208 | 204538308 | 466_106143_4194(MDM4)_8a | 0 | — |
| chr1 | 204544534 | 204544684 | 466_106145_4194(MDM4)_10a | 0 | — |
| chr1 | 204546796 | 204546877 | 466_106146_4194(MDM4)_11a | 0 | — |
| chr1 | 204549112 | 204549682 | 466_106147_4194(MDM4)_12a | 0 | — |
| chr12 | 68808949 | 68809086 | 466_106044_4193(MDM2)_2 | 0 | — |
| chr12 | 68828770 | 68828931 | 466_106051_4193(MDM2)_9 | 0 | — |
| chr12 | 68808477 | 68808491 | 466_106043_4193(MDM2)_1 | 0 | — |
| chr12 | 68809207 | 68809292 | 466_106045_4193(MDM2)_3 | 0 | — |
| chr12 | 68813553 | 68813628 | 466_106046_4193(MDM2)_4 | 0 | — |
| chr12 | 68816811 | 68816945 | 466_106047_4193(MDM2)_5 | 0 | — |
| chr12 | 68820324 | 68820374 | 466_106048_4193(MDM2)_6 | 0 | — |
| chr12 | 68824362 | 68824430 | 466_106049_4193(MDM2)_7 | 0 | — |
| chr12 | 68824554 | 68824651 | 466_106050_4193(MDM2)_8 | 0 | — |
| chr12 | 68835828 | 68835984 | 466_106052_4193(MDM2)_10 | 0 | — |
| chr12 | 68836671 | 68836749 | 466_106053_4193(MDM2)_11 | 0 | — |
| chr12 | 68839273 | 68839849 | 466_106054_4193(MDM2)_12 | 0 | — |
| chr4 | 55125226 | 55125293 | 464_88696_3791(KDR)_1a | 0 | — |
| chr4 | 55110402 | 55110566 | 464_88704_3791(KDR)_9a | 0 | — |
| chr4 | 55098136 | 55098272 | 464_88712_3791(KDR)_17a | 0 | — |
| chr4 | 55089373 | 55089473 | 464_88720_3791(KDR)_25a | 0 | — |
| chr5 | 171400152 | 171400210 | 469_119421_4869(NPM1)_7 | 0 | — |
| chr4 | 54285840 | 54285963 | 470_126855_5156(PDGFRA)_20a | 0 | — |
| chr4 | 54285370 | 54285486 | 470_126854_5156(PDGFRA)_19a | 0 | — |
| chr4 | 54280315 | 54280482 | 470_126852_5156(PDGFRA)_17a | 0 | — |
| chr4 | 54278361 | 54278515 | 470_126851_5156(PDGFRA)_16a | 0 | — |
| chr4 | 54277895 | 54278006 | 470_126850_5156(PDGFRA)_15a | 0 | — |
| chr4 | 54277387 | 54277492 | 470_126849_5156(PDGFRA)_14a | 0 | — |
| chr4 | 54274840 | 54274973 | 470_126848_5156(PDGFRA)_13a | 0 | — |
| chr4 | 54274530 | 54274625 | 470_126847_5156(PDGFRA)_12a | 0 | — |
| chr4 | 54273536 | 54273730 | 470_126846_5156(PDGFRA)_11a | 0 | — |
| chr4 | 54270632 | 54270748 | 470_126844_5156(PDGFRA)_9a | 0 | — |
| chr4 | 54267551 | 54267741 | 470_126843_5156(PDGFRA)_8a | 0 | — |
| chr4 | 54267288 | 54267460 | 470_126842_5156(PDGFRA)_7a | 0 | — |
| chr4 | 54264918 | 54265049 | 470_126841_5156(PDGFRA)_6a | 0 | — |
| chr4 | 54263666 | 54263927 | 470_126840_5156(PDGFRA)_5a | 0 | — |
| chr4 | 54261094 | 54261412 | 470_126839_5156(PDGFRA)_4a | 0 | — |
| chr4 | 54295124 | 54295272 | 470_126860_5156(PDGFRA)_25a | 0 | — |
| chr4 | 54289008 | 54289114 | 470_126858_5156(PDGFRA)_23a | 0 | — |
| chr4 | 54288798 | 54288898 | 470_126857_5156(PDGFRA)_22a | 0 | — |
| chr4 | 54287429 | 54287541 | 470_126856_5156(PDGFRA)_21a | 0 | — |
| chr4 | 54290312 | 54290554 | 470_126859_5156(PDGFRA)_24a | 0 | — |
| chr4 | 54281580 | 54281978 | 470_126853_5156(PDGFRA)_18a | 0 | — |
| chr4 | 54272393 | 54272520 | 470_126845_5156(PDGFRA)_10a | 0 | — |
| chr4 | 54258768 | 54258817 | 470_126838_5156(PDGFRA)_3a | 0 | — |
| chr7 | 129210964 | 129211113 | 474_160573_6608(SMO)_10a | 0 | — |
| chr7 | 129205202 | 129205412 | 474_160566_6608(SMO)_3a | 0 | — |
| chr3 | 10149786 | 10150367 | 477_191810_7428(VHL)_3 | 0 | — |
| chr3 | 10146513 | 10146636 | 477_191809_7428(VHL)_2 | 0 | — |
| chr3 | 10141847 | 10142187 | 477_191808_7428(VHL)_1 | 0 | — |
| chr7 | 116769644 | 116769791 | 466_106291_4233(MET)_15a | 0 | — |
| chr7 | 116740851 | 116741025 | 466_106284_4233(MET)_8a | 0 | — |
| chr3 | 37008813 | 37008905 | 466_106463_4292(MLH1)_8 | 0 | — |
| chr3 | 37006990 | 37007063 | 466_106462_4292(MLH1)_7 | 0 | — |
| chr3 | 37004400 | 37004474 | 466_106461_4292(MLH1)_6 | 0 | — |
| chr3 | 36997155 | 36997300 | 466_106459_4292(MLH1)_4 | 0 | — |
| chr3 | 36996618 | 36996709 | 466_106458_4292(MLH1)_3 | 0 | — |
| chr3 | 36993768 | 36993890 | 466_106457_4292(MLH1)_2 | 0 | — |
| chr3 | 36993547 | 36993663 | 466_106456_4292(MLH1)_1 | 0 | — |
| chr3 | 37048903 | 37049017 | 466_106475_4292(MLH1)_20 | 0 | — |
| chr3 | 37017505 | 37017599 | 466_106467_4292(MLH1)_12 | 0 | — |
| chr3 | 37000954 | 37001053 | 466_106460_4292(MLH1)_5 | 0 | — |
| chr1 | 43352517 | 43352772 | 466_106809_4352(MPL)_12a | 0 | — |
| chr1 | 43352215 | 43352303 | 466_106808_4352(MPL)_11a | 0 | — |
| chr1 | 43348842 | 43349002 | 466_106806_4352(MPL)_9a | 0 | — |
| chr1 | 43346791 | 43346934 | 466_106805_4352(MPL)_8a | 0 | — |
| chr1 | 43346444 | 43346629 | 466_106804_4352(MPL)_7a | 0 | — |
| chr1 | 43340386 | 43340513 | 466_106803_4352(MPL)_6a | 0 | — |
| chr1 | 43339963 | 43340126 | 466_106802_4352(MPL)_5a | 0 | — |
| chr1 | 43339270 | 43339569 | 466_106801_4352(MPL)_4a | 0 | — |
| chr1 | 43338098 | 43338231 | 466_106799_4352(MPL)_2a | 0 | — |
| chr1 | 43337848 | 43337927 | 466_106798_4352(MPL)_1a | 0 | — |
| chr1 | 43349262 | 43349359 | 466_106807_4352(MPL)_10a | 0 | — |
| chr1 | 43338541 | 43338720 | 466_106800_4352(MPL)_3a | 0 | — |
| chr9 | 5029782 | 5029906 | 463_81563_3717(JAK2)_4 | 0 | — |
| chr9 | 5054562 | 5054884 | 463_81649_3717(JAK2)_7 | 0 | — |
| chr9 | 5073697 | 5073785 | 463_81656_3717(JAK2)_14 | 0 | — |
| chr9 | 5090738 | 5090911 | 463_81664_3717(JAK2)_22 | 0 | — |
| chr9 | 5021987 | 5022213 | 463_81562_3717(JAK2)_3 | 0 | — |
| chr9 | 5044402 | 5044520 | 463_81564_3717(JAK2)_5 | 0 | — |
| chr1 | 64883276 | 64883475 | 463_81625_3716(JAK1)_4a | 0 | — |
| chr1 | 64855508 | 64855698 | 463_81633_3716(JAK1)_12a | 0 | — |
| chr1 | 64841450 | 64841601 | 463_81640_3716(JAK1)_19a | 0 | — |
| chr1 | 64844063 | 64844215 | 463_81639_3716(JAK1)_18a | 0 | — |
| chr1 | 64841244 | 64841339 | 463_81641_3716(JAK1)_20a | 0 | — |
| chr7 | 116795654 | 116795791 | 466_106299_4233(MET)_23a | 0 | — |
| chr1 | 64839602 | 64839795 | 463_81642_3716(JAK1)_21a | 0 | — |
| chr4 | 55115280 | 55115411 | 464_88699_3791(KDR)_4a | 0 | — |
| chr4 | 55114873 | 55115042 | 464_88700_3791(KDR)_5a | 0 | — |
| chr4 | 55114125 | 55114265 | 464_88701_3791(KDR)_6a | 0 | — |
| chr4 | 55113303 | 55113481 | 464_88702_3791(KDR)_7a | 0 | — |
| chr4 | 55110653 | 55110768 | 464_88703_3791(KDR)_8a | 0 | — |
| chr4 | 55107736 | 55107893 | 464_88705_3791(KDR)_10a | 0 | — |
| chr4 | 55106686 | 55106810 | 464_88706_3791(KDR)_11a | 0 | — |
| chr4 | 55105831 | 55105940 | 464_88707_3791(KDR)_12a | 0 | — |
| chr4 | 55104642 | 55104984 | 464_88708_3791(KDR)_13a | 0 | — |
| chr4 | 55102361 | 55102508 | 464_88709_3791(KDR)_14a | 0 | — |
| chr4 | 55101896 | 55102028 | 464_88710_3791(KDR)_15a | 0 | — |
| chr4 | 55098696 | 55098803 | 464_88711_3791(KDR)_16a | 0 | — |
| chr4 | 55097661 | 55097766 | 464_88713_3791(KDR)_18a | 0 | — |
| chr4 | 55096228 | 55096342 | 464_88714_3791(KDR)_19a | 0 | — |
| chr4 | 55095576 | 55095665 | 464_88715_3791(KDR)_20a | 0 | — |
| chr4 | 55094801 | 55094955 | 464_88716_3791(KDR)_21a | 0 | — |
| chr4 | 55092616 | 55092714 | 464_88717_3791(KDR)_22a | 0 | — |
| chr4 | 55089955 | 55090078 | 464_88718_3791(KDR)_23a | 0 | — |
| chr4 | 55089690 | 55089802 | 464_88719_3791(KDR)_24a | 0 | — |
| chr12 | 120988832 | 120989032 | 463_81839_6927(HNF1A)_2 | 0 | — |
| chr12 | 120993519 | 120993706 | 463_81840_6927(HNF1A)_3 | 0 | — |
| chr12 | 120994163 | 120994405 | 463_81841_6927(HNF1A)_4 | 0 | — |
| chr12 | 120996261 | 120996413 | 463_81842_6927(HNF1A)_5 | 0 | — |
| chr12 | 120996540 | 120997166 | 463_81843_6927(HNF1A)_6 | 0 | — |
| chr22 | 23793558 | 23793688 | 474_160330_6598(SMARCB)_3 | 0 | — |
| chr4 | 55088867 | 55088973 | 464_88721_3791(KDR)_26a | 0 | — |
| chr4 | 55087606 | 55087758 | 464_88722_3791(KDR)_27a | 0 | — |
| chr4 | 55082535 | 55082635 | 464_88723_3791(KDR)_28a | 0 | — |
| chr4 | 55081955 | 55082041 | 464_88724_3791(KDR)_29a | 0 | — |
| chr4 | 55079940 | 55080163 | 464_88725_3791(KDR)_30a | 0 | — |
| chr12 | 120997473 | 120997665 | 463_81844_6927(HNF1A)_7 | 0 | — |
| chr12 | 121001064 | 121001192 | 463_81846_6927(HNF1A)_9 | 0 | — |
| chr16 | 2088445 | 2088610 | 476_182379_7249(TSC2)_42 | 0 | — |
| chr16 | 2088226 | 2088325 | 476_182378_7249(TSC2)_41 | 0 | — |
| chr16 | 2088047 | 2088139 | 476_182377_7249(TSC2)_40 | 0 | — |
| chr16 | 2087862 | 2087941 | 476_182376_7249(TSC2)_39 | 0 | — |
| chr16 | 2086731 | 2086871 | 476_182375_7249(TSC2)_38 | 0 | — |
| chr16 | 2085229 | 2085322 | 476_182373_7249(TSC2)_36 | 0 | — |
| chr16 | 2084950 | 2085026 | 476_182372_7249(TSC2)_35 | 0 | — |
| chr16 | 2084227 | 2084715 | 476_182371_7249(TSC2)_34 | 0 | — |
| chr16 | 2083694 | 2083816 | 476_182370_7249(TSC2)_33 | 0 | — |
| chr16 | 2082435 | 2082504 | 476_182369_7249(TSC2)_32 | 0 | — |
| chr16 | 2081594 | 2081798 | 476_182368_7249(TSC2)_31 | 0 | — |
| chr16 | 2079556 | 2079669 | 476_182366_7249(TSC2)_29 | 0 | — |
| chr16 | 2079275 | 2079428 | 476_182365_7249(TSC2)_28 | 0 | — |
| chr16 | 2079031 | 2079196 | 476_182364_7249(TSC2)_27 | 0 | — |
| chr16 | 2077597 | 2077726 | 476_182363_7249(TSC2)_26 | 0 | — |
| chr16 | 2076490 | 2076585 | 476_182362_7249(TSC2)_25 | 0 | — |
| chr16 | 2076067 | 2076257 | 476_182361_7249(TSC2)_24 | 0 | — |
| chr16 | 2074199 | 2074389 | 476_182359_7249(TSC2)_22 | 0 | — |
| chr16 | 2072848 | 2072983 | 476_182358_7249(TSC2)_21 | 0 | — |
| chr16 | 2072240 | 2072363 | 476_182357_7249(TSC2)_20 | 0 | — |
| chr16 | 2071783 | 2071934 | 476_182356_7249(TSC2)_19 | 0 | — |
| chr16 | 2071509 | 2071616 | 476_182355_7249(TSC2)_18 | 0 | — |
| chr16 | 2070455 | 2070578 | 476_182354_7249(TSC2)_17 | 0 | — |
| chr16 | 2064271 | 2064427 | 476_182352_7249(TSC2)_15 | 0 | — |
| chr16 | 2062971 | 2063053 | 476_182351_7249(TSC2)_14 | 0 | — |
| chr16 | 2062496 | 2062600 | 476_182350_7249(TSC2)_13 | 0 | — |
| chr16 | 2061870 | 2062008 | 476_182349_7249(TSC2)_12 | 0 | — |
| chr16 | 2060669 | 2060813 | 476_182348_7249(TSC2)_11 | 0 | — |
| chr4 | 55121096 | 55121190 | 464_88697_3791(KDR)_2a | 0 | — |
| chr4 | 55118603 | 55118800 | 464_88698_3791(KDR)_3a | 0 | — |
| chr15 | 90088358 | 90088502 | 463_79505_3418(IDH2)_6a | 0 | — |
| chr15 | 90087438 | 90087575 | 463_79506_3418(IDH2)_7a | 0 | — |
| chr15 | 90087111 | 90087263 | 463_79507_3418(IDH2)_8a | 0 | — |
| chr15 | 90085274 | 90085387 | 463_79508_3418(IDH2)_9a | 0 | — |
| chr15 | 90085000 | 90085098 | 463_79509_3418(IDH2)_10a | 0 | — |
| chr15 | 90084265 | 90084353 | 463_79511_3418(IDH2)_12a | 0 | — |
| chr15 | 90102275 | 90102390 | 463_79500_3418(IDH2)_1a | 0 | — |
| chr15 | 90100575 | 90100690 | 463_79501_3418(IDH2)_2a | 0 | — |
| chr15 | 90091552 | 90091644 | 463_79502_3418(IDH2)_3a | 0 | — |
| chr15 | 90090478 | 90090644 | 463_79503_3418(IDH2)_4a | 0 | — |
| chr2 | 208251429 | 208251551 | 463_79492_3417(IDH1)_6a | 0 | — |
| chr2 | 208248368 | 208248660 | 463_79493_3417(IDH1)_7a | 0 | — |
| chr2 | 208245318 | 208245424 | 463_79494_3417(IDH1)_8a | 0 | — |
| chr2 | 208243426 | 208243604 | 463_79495_3417(IDH1)_9a | 0 | — |
| chr2 | 208239862 | 208240003 | 463_79497_3417(IDH1)_11a | 0 | — |
| chr2 | 208239070 | 208239233 | 463_79498_3417(IDH1)_12a | 0 | — |
| chr2 | 208237078 | 208237169 | 463_79499_3417(IDH1)_13a | 0 | — |
| chr11 | 533765 | 533944 | 463_79035_3265(HRAS)_3a | 0 | — |
| chr11 | 533452 | 533612 | 463_79036_3265(HRAS)_4a | 0 | — |
| chr11 | 533276 | 533358 | 463_79037_3265(HRAS)_5a | 0 | — |
| chr11 | 532588 | 532755 | 463_79038_3265(HRAS)_6a | 0 | — |
| chr10 | 43112852 | 43112963 | 473_148927_5979(RET)_9a | 0 | — |
| chr10 | 43112098 | 43112224 | 473_148926_5979(RET)_8a | 0 | — |
| chr10 | 8055655 | 8055896 | 462_69261_2625(GATA3)_4a | 0 | — |
| chr10 | 8058304 | 8058841 | 462_69262_2625(GATA3)_5a | 0 | — |
| chr10 | 8063992 | 8064138 | 462_69263_2625(GATA3)_6a | 0 | — |
| chr10 | 8069472 | 8069598 | 462_69264_2625(GATA3)_7a | 0 | — |
| chr10 | 8073738 | 8074023 | 462_69265_2625(GATA3)_8a | 0 | — |
| chr19 | 17844233 | 17844430 | 463_81744_3718(JAK3)_2a | 0 | — |
| chr19 | 17843776 | 17843900 | 463_81745_3718(JAK3)_3a | 0 | — |
| chr19 | 17843379 | 17843491 | 463_81746_3718(JAK3)_4a | 0 | — |
| chr19 | 17843026 | 17843172 | 463_81747_3718(JAK3)_5a | 0 | — |
| chr19 | 17842315 | 17842610 | 463_81748_3718(JAK3)_6a | 0 | — |
| chr19 | 17841639 | 17841762 | 463_81749_3718(JAK3)_7a | 0 | — |
| chr19 | 17840229 | 17840341 | 463_81751_3718(JAK3)_9a | 0 | — |
| chr19 | 17839476 | 17839663 | 463_81752_3718(JAK3)_10a | 0 | — |
| chr19 | 17838262 | 17838390 | 463_81753_3718(JAK3)_11a | 0 | — |
| chr19 | 17837931 | 17838063 | 463_81754_3718(JAK3)_12a | 0 | — |
| chr19 | 17837128 | 17837213 | 463_81755_3718(JAK3)_13a | 0 | — |
| chr19 | 17835923 | 17836051 | 463_81756_3718(JAK3)_14a | 0 | — |
| chr19 | 17835082 | 17835215 | 463_81757_3718(JAK3)_15a | 0 | — |
| chr19 | 17834570 | 17834721 | 463_81759_3718(JAK3)_17a | 0 | — |
| chr19 | 17832789 | 17832929 | 463_81760_3718(JAK3)_18a | 0 | — |
| chr19 | 17832518 | 17832708 | 463_81761_3718(JAK3)_19a | 0 | — |
| chr19 | 17831673 | 17831798 | 463_81762_3718(JAK3)_20a | 0 | — |
| chr19 | 17831227 | 17831400 | 463_81763_3718(JAK3)_21a | 0 | — |
| chr19 | 17830502 | 17830620 | 463_81764_3718(JAK3)_22a | 0 | — |
| chr19 | 17830107 | 17830218 | 463_81765_3718(JAK3)_23a | 0 | — |
| chr4 | 152350041 | 152350124 | 461_64086_55294(FBXW7)_7 | 0 | — |
| chr4 | 152324183 | 152324394 | 461_64095_55294(FBXW7)_16 | 0 | — |
| chr10 | 43128111 | 43128269 | 473_148938_5979(RET)_20a | 0 | — |
| chr10 | 43126574 | 43127504 | 473_148937_5979(RET)_19a | 0 | — |
| chr10 | 43123670 | 43123808 | 473_148935_5979(RET)_17a | 0 | — |
| chr10 | 43121945 | 43122016 | 473_148934_5979(RET)_16a | 0 | — |
| chr10 | 43120080 | 43120203 | 473_148933_5979(RET)_15a | 0 | — |
| chr10 | 43119530 | 43119745 | 473_148932_5979(RET)_14a | 0 | — |
| chr10 | 43118372 | 43118480 | 473_148931_5979(RET)_13a | 0 | — |
| chr10 | 43116583 | 43116731 | 473_148930_5979(RET)_12a | 0 | — |
| chr10 | 43113555 | 43113675 | 473_148928_5979(RET)_10a | 0 | — |
| chr19 | 17847945 | 17848061 | 463_81743_3718(JAK3)_1a | 0 | — |
| chr13 | 48477354 | 48477404 | 472_141468_5925(RB1)_26a | 0 | — |
| chr13 | 48476700 | 48476843 | 472_141467_5925(RB1)_25a | 0 | — |
| chr13 | 48473359 | 48473390 | 472_141466_5925(RB1)_24a | 0 | — |
| chr13 | 48465204 | 48465368 | 472_141465_5925(RB1)_23a | 0 | — |
| chr13 | 48464997 | 48465111 | 472_141464_5925(RB1)_22a | 0 | — |
| chr13 | 48463730 | 48463835 | 472_141463_5925(RB1)_21a | 0 | — |
| chr13 | 48456203 | 48456349 | 472_141461_5925(RB1)_19a | 0 | — |
| chr13 | 48452992 | 48453111 | 472_141460_5925(RB1)_18a | 0 | — |
| chr13 | 48381246 | 48381443 | 472_141459_5925(RB1)_17a | 0 | — |
| chr13 | 48380164 | 48380241 | 472_141458_5925(RB1)_16a | 0 | — |
| chr13 | 48380052 | 48380084 | 472_141457_5925(RB1)_15a | 0 | — |
| chr13 | 48379593 | 48379650 | 472_141456_5925(RB1)_14a | 0 | — |
| chr13 | 48376917 | 48377034 | 472_141455_5925(RB1)_13a | 0 | — |
| chr13 | 48368526 | 48368604 | 472_141453_5925(RB1)_11a | 0 | — |
| chr13 | 48367493 | 48367603 | 472_141452_5925(RB1)_10a | 0 | — |
| chr13 | 48364893 | 48364971 | 472_141451_5925(RB1)_9a | 0 | — |
| chr13 | 48362814 | 48362957 | 472_141450_5925(RB1)_8a | 0 | — |
| chr13 | 48360016 | 48360127 | 472_141449_5925(RB1)_7a | 0 | — |
| chr13 | 48348955 | 48349023 | 472_141448_5925(RB1)_6a | 0 | — |
| chr13 | 48345079 | 48345199 | 472_141446_5925(RB1)_4a | 0 | — |
| chr13 | 48342598 | 48342714 | 472_141445_5925(RB1)_3a | 0 | — |
| chr13 | 48307279 | 48307406 | 472_141444_5925(RB1)_2a | 0 | — |
| chr13 | 48303912 | 48304049 | 472_141443_5925(RB1)_1a | 0 | — |
| chr4 | 152411159 | 152411872 | 461_64083_55294(FBXW7)_4 | 0 | — |
| chr4 | 152382188 | 152382512 | 461_64084_55294(FBXW7)_5 | 0 | — |
| chr4 | 152352469 | 152352958 | 461_64085_55294(FBXW7)6 | 0 | — |
| chr4 | 152348673 | 152348801 | 461_64087_55294(FBXW7)_8 | 0 | — |
| chr4 | 152346929 | 152347071 | 461_64088_55294(FBXW7)_9 | 0 | — |
| chr4 | 152337801 | 152337936 | 461_64089_55294(FBXW7)_10 | 0 | — |
| chr4 | 152332595 | 152332719 | 461_64090_55294(FBXW7)_11 | 0 | — |
| chr4 | 152330731 | 152330868 | 461_64091_55294(FBXW7)_12 | 0 | — |
| chr4 | 152329671 | 152329785 | 461_64092_55294(FBXW7)_13 | 0 | — |
| chr4 | 152328207 | 152328389 | 461_64093_55294(FBXW7)_14 | 0 | — |
| chr4 | 152326005 | 152326231 | 461_64094_55294(FBXW7)_15 | 0 | — |
| chr4 | 152323088 | 152323149 | 461_64096_55294(FBXW7)_17 | 0 | — |
| chr1 | 26762151 | 26762319 | 426_4224_8289(ARID1A)_7a | 0 | — |
| chr1 | 26773345 | 26773496 | 426_4232_8289(ARID1A)_15a | 0 | — |
| chr10 | 43111206 | 43111465 | 473_148925_5979(RET)_7a | 0 | — |
| chr10 | 43109030 | 43109230 | 473_148924_5979(RET)_6a | 0 | — |
| chr10 | 43106375 | 43106571 | 473_148923_5979(RET)_5a | 0 | — |
| chr10 | 43102341 | 43102629 | 473_148921_5979(RET)_3a | 0 | — |
| chr10 | 43100458 | 43100722 | 473_148920_5979(RET)_2a | 0 | — |
| chr10 | 43077258 | 43077331 | 473_148919_5979(RET)_1a | 0 | — |
| chr13 | 32326100 | 32326150 | 427_15653_675(BRCA2)_5 | 0 | — |
| chr13 | 32346826 | 32346896 | 427_15661_675(BRCA2)_13 | 0 | — |
| chr17 | 43097243 | 43097289 | 427_15613_672(BRCA1)_8 | 0 | — |
| chr17 | 43070927 | 43071238 | 427_15621_672(BRCA1)_16 | 0 | — |
| chr17 | 43045677 | 43045802 | 427_15629_672(BRCA1)_24 | 0 | — |
| chr7 | 140794307 | 140794467 | 427_15637_673(BRAF)_8 | 0 | — |
| chr7 | 140749286 | 140749418 | 427_15645_673(BRAF)_16 | 0 | — |
| chr13 | 32376669 | 32376791 | 427_15669_675(BRCA2)_21 | 0 | — |
| chr18 | 51049294 | 51049324 | 474_158718_4089(SMAD4)_4 | 0 | — |
| chr19 | 2164184 | 2164265 | 431_55601_84444(DOT1L)_1 | 0 | — |
| chr22 | 29043298 | 29043430 | 478_203546_84133(ZNRF3)_7 | 0 | — |
| chr22 | 29044779 | 29044890 | 478_203547_84133(ZNRF3)_8 | 0 | — |
| chr22 | 29046715 | 29046883 | 478_203548_84133(ZNRF3)_9 | 0 | — |
| chr22 | 29049196 | 29050948 | 478_203550_84133(ZNRF3)_11 | 0 | — |
| chr22 | 29053578 | 29053622 | 478_203551_84133(ZNRF3)_12 | 0 | — |
| chr19 | 2180712 | 2180756 | 431_55602_84444(DOT1L)_2 | 0 | — |
| chr4 | 1805354 | 1805476 | 461_60345_2261(FGFR3)_12a | 0 | — |
| chr4 | 1805558 | 1805669 | 461_60346_2261(FGFR3)_13a | 0 | — |
| chr4 | 1805749 | 1805940 | 461_60347_2261(FGFR3)_14a | 0 | — |
| chr4 | 1806050 | 1806173 | 461_60348_2261(FGFR3)_15a | 0 | — |
| chr4 | 1806256 | 1806327 | 461_60349_2261(FGFR3)_16a | 0 | — |
| chr4 | 1806545 | 1806683 | 461_60350_2261(FGFR3)_17a | 0 | — |
| chr4 | 1807115 | 1807262 | 461_60352_2261(FGFR3)_19a | 0 | — |
| chr4 | 1793934 | 1794043 | 461_60335_2261(FGFR3)_2a | 0 | — |
| chr4 | 1799253 | 1799523 | 461_60336_2261(FGFR3)_3a | 0 | — |
| chr4 | 1799746 | 1799812 | 461_60337_2261(FGFR3)_4a | 0 | — |
| chr4 | 1801366 | 1801536 | 461_60338_2261(FGFR3)_5a | 0 | — |
| chr19 | 2185854 | 2185929 | 431_55603_84444(DOT1L)_3 | 0 | — |
| chr19 | 2189731 | 2189795 | 431_55604_84444(DOT1L)_4 | 0 | — |
| chr19 | 2191011 | 2191240 | 431_55605_84444(DOT1L)_5 | 0 | — |
| chr19 | 2193688 | 2193783 | 431_55606_84444(DOT1L)_6 | 0 | — |
| chr19 | 2199883 | 2199939 | 431_55608_84444(DOT1L)_8 | 0 | — |
| chr19 | 2202699 | 2202779 | 431_55609_84444(DOT1L)_9 | 0 | — |
| chr19 | 2206728 | 2206797 | 431_55610_84444(DOT1L)_10 | 0 | — |
| chr19 | 2207573 | 2207680 | 431_55611_84444(DOT1L)_11 | 0 | — |
| chr19 | 2208934 | 2208976 | 431_55612_84444(DOT1L)_12 | 0 | — |
| chr19 | 2210399 | 2210510 | 431_55613_84444(DOT1L)_13 | 0 | — |
| chr19 | 2210620 | 2210855 | 431_55614_84444(DOT1L)_14 | 0 | — |
| chr19 | 2211750 | 2211842 | 431_55616_84444(DOT1L)_16 | 0 | — |
| chr19 | 2213538 | 2213640 | 431_55617_84444(DOT1L)_17 | 0 | — |
| chr19 | 2213848 | 2213986 | 431_55618_84444(DOT1L)_18 | 0 | — |
| chr19 | 2214470 | 2214596 | 431_55619_84444(DOT1L)_19 | 0 | — |
| chr19 | 2216280 | 2216765 | 431_55620_84444(DOT1L)_20 | 0 | — |
| chr15 | 66444655 | 66444707 | 465_99854_5604(MAP2K1)_6a | 0 | — |
| chr15 | 66481754 | 66481879 | 465_99855_5604(MAP2K1)_7a | 0 | — |
| chr15 | 66484989 | 66485191 | 465_99856_5604(MAP2K1)_8a | 0 | — |
| chr15 | 66487227 | 66487292 | 465_99857_5604(MAP2K1)_9a | 0 | — |
| chr15 | 66489214 | 66489276 | 465_99858_5604(MAP2K1)_10a | 0 | — |
| chr15 | 66489717 | 66489763 | 465_99859_5604(MAP2K1)_11a | 0 | — |
| chr19 | 2216954 | 2217090 | 431_55621_84444(DOT1L)_21 | 0 | — |
| chr19 | 2217771 | 2217918 | 431_55622_84444(DOT1L)_22 | 0 | — |
| chr19 | 2221975 | 2222559 | 431_55624_84444(DOT1L)_24 | 0 | — |
| chr19 | 2223280 | 2223486 | 431_55625_84444(DOT1L)_25 | 0 | — |
| chr19 | 2225387 | 2225452 | 431_55626_84444(DOT1L)_26 | 0 | — |
| chr19 | 2226182 | 2227127 | 431_55627_84444(DOT1L)_27 | 0 | — |
| chr19 | 2227736 | 2229792 | 431_55628_84444(DOT1L)_28 | 0 | — |
| chr9 | 5050685 | 5050831 | 463_81648_3717(JAK2)_6 | 0 | — |
| chr9 | 5055668 | 5055788 | 463_81650_3717(JAK2)_8 | 0 | — |
| chr9 | 5064882 | 5065040 | 463_81651_3717(JAK2)_9 | 0 | — |
| chr9 | 5066677 | 5066789 | 463_81652_3717(JAK2)_10 | 0 | — |
| chr9 | 5069021 | 5069208 | 463_81653_3717(JAK2)_11 | 0 | — |
| chr9 | 5069924 | 5070052 | 463_81654_3717(JAK2)_12 | 0 | — |
| chr9 | 5072491 | 5072626 | 463_81655_3717(JAK2)_13 | 0 | — |
| chr9 | 5077452 | 5077580 | 463_81657_3717(JAK2)_15 | 0 | — |
| chr9 | 5078305 | 5078444 | 463_81658_3717(JAK2)_16 | 0 | — |
| chr9 | 5080228 | 5080380 | 463_81659_3717(JAK2)_17 | 0 | — |
| chr9 | 5080532 | 5080683 | 463_81660_3717(JAK2)_18 | 0 | — |
| chr9 | 5081724 | 5081861 | 463_81661_3717(JAK2)_19 | 0 | — |
| chr9 | 5089673 | 5089863 | 463_81662_3717(JAK2)_20 | 0 | — |
| chr9 | 5090445 | 5090570 | 463_81663_3717(JAK2)_21 | 0 | — |
| chr9 | 5123003 | 5123121 | 463_81665_3717(JAK2)_23 | 0 | — |
| chr9 | 5126332 | 5126446 | 463_81666_3717(JAK2)_24 | 0 | — |
| chr9 | 5126683 | 5126791 | 463_81667_3717(JAK2)_25 | 0 | — |
| chr4 | 1801619 | 1801743 | 461_60339_2261(FGFR3)_6a | 0 | — |
| chr4 | 1801834 | 1802025 | 461_60340_2261(FGFR3)_7a | 0 | — |
| chr10 | 121518681 | 121518829 | 461_60359_2263(FGFR2)_9a | 0 | — |
| chr10 | 121487353 | 121487424 | 461_60367_2263(FGFR2)_17a | 0 | — |
| chr10 | 121593708 | 121593817 | 461_60353_2263(FGFR2)_3a | 0 | — |
| chr10 | 121565437 | 121565704 | 461_60354_2263(FGFR2)_4a | 0 | — |
| chr10 | 121564501 | 121564579 | 461_60355_2263(FGFR2)_5a | 0 | — |
| chr10 | 121551289 | 121551459 | 461_60356_2263(FGFR2)_6a | 0 | — |
| chr10 | 121538591 | 121538715 | 461_60357_2263(FGFR2)_7a | 0 | — |
| chr10 | 121519978 | 121520169 | 461_60358_2263(FGFR2)_8a | 0 | — |
| chr10 | 121517318 | 121517463 | 461_60360_2263(FGFR2)_10a | 0 | — |
| chr10 | 121515116 | 121515319 | 461_60361_2263(FGFR2)_11a | 0 | — |
| chr10 | 121503789 | 121503941 | 461_60362_2263(FGFR2)_12a | 0 | — |
| chr10 | 121500825 | 121500947 | 461_60363_2263(FGFR2)_13a | 0 | — |
| chr10 | 121498494 | 121498605 | 461_60364_2263(FGFR2)_14a | 0 | — |
| chr10 | 121496531 | 121496722 | 461_60365_2263(FGFR2)_15a | 0 | — |
| chr10 | 121487990 | 121488113 | 461_60366_2263(FGFR2)_16a | 0 | — |
| chr10 | 121485394 | 121485532 | 461_60368_2263(FGFR2)_18a | 0 | — |
| chr10 | 121483697 | 121483803 | 461_60369_2263(FGFR2)_19a | 0 | — |
| chr10 | 121481850 | 121482177 | 461_60370_2263(FGFR2)_20a | 0 | — |
| chr10 | 121479580 | 121480021 | 461_60371_2263(FGFR2)_21a | 0 | — |
| chr8 | 38461095 | 38461106 | 461_60316_2260(FGFR1)_3a | 0 | — |
| chr8 | 38421796 | 38421941 | 461_60324_2260(FGFR1)_11a | 0 | — |
| chr8 | 38413917 | 38414023 | 461_60333_2260(FGFR1)_20a | 0 | — |
| chr8 | 38457355 | 38457534 | 461_60317_2260(FGFR1)_4a | 0 | — |
| chr8 | 38440305 | 38440373 | 461_60318_2260(FGFR1)_5a | 0 | — |
| chr8 | 38429681 | 38429948 | 461_60319_2260(FGFR1)_6a | 0 | — |
| chr8 | 38428345 | 38428435 | 461_60320_2260(FGFR1)_7a | 0 | — |
| chr8 | 38427920 | 38428093 | 461_60321_2260(FGFR1)_8a | 0 | — |
| chr8 | 38426121 | 38426245 | 461_60322_2260(FGFR1)_9a | 0 | — |
| chr8 | 38424508 | 38424699 | 461_60323_2260(FGFR1)_10a | 0 | — |
| chr8 | 38419532 | 38419735 | 461_60325_2260(FGFR1)_12a | 0 | — |
| chr8 | 38418227 | 38418373 | 461_60326_2260(FGFR1)_13a | 0 | — |
| chr8 | 38417869 | 38417991 | 461_60327_2260(FGFR1)_14a | 0 | — |
| chr8 | 38417305 | 38417416 | 461_60328_2260(FGFR1)_15a | 0 | — |
| chr8 | 38415869 | 38416060 | 461_60329_2260(FGFR1)_16a | 0 | — |
| chr8 | 38414778 | 38414901 | 461_60330_2260(FGFR1)_17a | 0 | — |
| chr1 | 64838464 | 64838589 | 463_81643_3716(JAK1)_22a | 0 | — |
| chr1 | 64837931 | 64838104 | 463_81644_3716(JAK1)_23a | 0 | — |
| chr1 | 64836097 | 64836215 | 463_81645_3716(JAK1)_24a | 0 | — |
| chr1 | 64835395 | 64835506 | 463_81646_3716(JAK1)_25a | 0 | — |
| chr8 | 38414558 | 38414629 | 461_60331_2260(FGFR1)_18a | 0 | — |
| chr8 | 38414151 | 38414289 | 461_60332_2260(FGFR1)_19a | 0 | — |
| chr8 | 38413627 | 38413804 | 461_60334_2260(FGFR1)_21a | 0 | — |
| chr7 | 148832633 | 148832750 | 461_59349_2146(EZH2)_6 | 0 | — |
| chr7 | 148816683 | 148816778 | 461_59357_2146(EZH2)_14 | 0 | — |
| chr1 | 64834561 | 64834657 | 463_81647_3716(JAK1)_26a | 0 | — |
| chr1 | 64886258 | 64886341 | 463_81624_3716(JAK1)_3a | 0 | — |
| chr1 | 64879024 | 64879148 | 463_81626_3716(JAK1)_5a | 0 | — |
| chr1 | 64873369 | 64873523 | 463_81627_3716(JAK1)_6a | 0 | — |
| chr22 | 29031584 | 29031631 | 478_203544_84133(ZNRF3)_5 | 0 | — |
| chr7 | 148807645 | 148807706 | 461_59365_2146(EZH2)_22 | 0 | — |
| chr7 | 148850453 | 148850470 | 461_59346_2146(EZH2)_3 | 0 | — |
| chr7 | 148847181 | 148847305 | 461_59347_2146(EZH2)_4 | 0 | — |
| chr7 | 148846469 | 148846598 | 461_59348_2146(EZH2)_5 | 0 | — |
| chr7 | 148829727 | 148829848 | 461_59350_2146(EZH2)_7 | 0 | — |
| chr7 | 148828739 | 148828880 | 461_59351_2146(EZH2)_8 | 0 | — |
| chr7 | 148827163 | 148827266 | 461_59352_2146(EZH2)_9 | 0 | — |
| chr7 | 148826453 | 148826632 | 461_59353_2146(EZH2)_10 | 0 | — |
| chr7 | 148819595 | 148819687 | 461_59354_2146(EZH2)_11 | 0 | — |
| chr7 | 148817876 | 148818117 | 461_59355_2146(EZH2)_12 | 0 | — |
| chr7 | 148817221 | 148817391 | 461_59356_2146(EZH2)_13 | 0 | — |
| chr7 | 148815505 | 148815546 | 461_59358_2146(EZH2)_15 | 0 | — |
| chr7 | 148814913 | 148815039 | 461_59359_2146(EZH2)_16 | 0 | — |
| chr7 | 148813958 | 148814137 | 461_59360_2146(EZH2)_17 | 0 | — |
| chr7 | 148811624 | 148811720 | 461_59361_2146(EZH2)_18 | 0 | — |
| chr7 | 148810332 | 148810414 | 461_59362_2146(EZH2)_19 | 0 | — |
| chr7 | 148809309 | 148809390 | 461_59363_2146(EZH2)_20 | 0 | — |
| chr7 | 148809070 | 148809155 | 461_59364_2146(EZH2)_21 | 0 | — |
| chr1 | 64869310 | 64869474 | 463_81628_3716(JAK1)_7a | 0 | — |
| chr1 | 64866865 | 64867208 | 463_81629_3716(JAK1)_8a | 0 | — |
| chr1 | 64864786 | 64864972 | 463_81630_3716(JAK1)_9a | 0 | — |
| chr1 | 64860104 | 64860262 | 463_81631_3716(JAK1)_10a | 0 | — |
| chr1 | 64857655 | 64857779 | 463_81632_3716(JAK1)_11a | 0 | — |
| chr1 | 64850803 | 64850910 | 463_81634_3716(JAK1)_13a | 0 | — |
| chr1 | 64847531 | 64847675 | 463_81635_3716(JAK1)_14a | 0 | — |
| chr1 | 64846648 | 64846736 | 463_81636_3716(JAK1)_15a | 0 | — |
| chr1 | 64845512 | 64845640 | 463_81637_3716(JAK1)_16a | 0 | — |
| chr1 | 64844753 | 64844889 | 463_81638_3716(JAK1)_17a | 0 | — |
| chr15 | 90088586 | 90088747 | 463_79504_3418(IDH2)_5a | 0 | — |
| chr15 | 90084815 | 90084908 | 463_79510_3418(IDH2)_11a | 0 | — |
| chr2 | 208241993 | 208242145 | 463_79496_3417(IDH1)_10a | 0 | — |
| chr11 | 534211 | 534322 | 463_79034_3265(HRAS)_2a | 0 | — |
| chr13 | 48479997 | 48480071 | 472_141469_5925(RB1)_27a | 0 | — |
| chr13 | 48459687 | 48459833 | 472_141462_5925(RB1)_20a | 0 | — |
| chr13 | 48373404 | 48373492 | 472_141454_5925(RB1)_12a | 0 | — |
| chr13 | 48347824 | 48347863 | 472_141447_5925(RB1)_5a | 0 | — |
| chr17 | 58354942 | 58354986 | 473_154862_54894(RNF43)_10 | 0 | — |
| chr17 | 58357467 | 58358823 | 473_154861_54894(RNF43)_9 | 0 | — |
| chr17 | 58360148 | 58360251 | 473_154860_54894(RNF43)_8 | 0 | — |
| chr17 | 58360782 | 58360944 | 473_154859_54894(RNF43)_7 | 0 | — |
| chr17 | 58362543 | 58362648 | 473_154858_54894(RNF43)_6 | 0 | — |
| chr17 | 58363274 | 58363406 | 473_154857_54894(RNF43)_5 | 0 | — |
| chr17 | 58370910 | 58371033 | 473_154855_54894(RNF43)_3 | 0 | — |
| chr17 | 58415325 | 58415577 | 473_154854_54894(RNF43)_2 | 0 | — |
| chr22 | 29042494 | 29042569 | 478_203545_84133(ZNRF3)_6 | 0 | — |
| chr22 | 23834140 | 23834180 | 474_160336_6598(SMARCB1)_9 | 0 | — |
| chr22 | 23833571 | 23833703 | 474_160335_6598(SMARCB1)_8 | 0 | — |
| chr22 | 23825224 | 23825415 | 474_160334_6598(SMARCB1)_7 | 0 | — |
| chr22 | 23816769 | 23816936 | 474_160333_6598(SMARCB1)_6 | 0 | — |
| chr22 | 23803294 | 23803422 | 474_160332_6598(SMARCB1)_5 | 0 | — |
| chr22 | 23800943 | 23801135 | 474_160331_6598(SMARCB1)_4 | 0 | — |
| chr22 | 23791755 | 23791894 | 474_160329_6598(SMARCB1)_2 | 0 | — |
| chr22 | 23787169 | 23787262 | 474_160328_6598(SMARCB1)_1 | 0 | — |
| chr7 | 129212023 | 129212451 | 474_160575_6608(SMO)_12a | 0 | — |
| chr7 | 129211635 | 129211770 | 474_160574_6608(SMO)_11a | 0 | — |
| chr7 | 129210362 | 129210548 | 474_160572_6608(SMO)_9a | 0 | — |
| chr7 | 129209288 | 129209397 | 474_160571_6608(SMO)_8a | 0 | — |
| chr7 | 129208758 | 129208851 | 474_160570_6608(SMO)_7a | 0 | — |
| chr7 | 129206463 | 129206587 | 474_160569_6608(SMO)_6a | 0 | — |
| chr7 | 129206149 | 129206369 | 474_160568_6608(SMO)_5a | 0 | — |
| chr7 | 129205609 | 129205782 | 474_160567_6608(SMO)_4a | 0 | — |
| chr7 | 129203383 | 129203589 | 474_160565_6608(SMO)_2a | 0 | — |
| chr7 | 129189151 | 129189482 | 474_160564_6608(SMO)_1a | 0 | — |
| chr22 | 28917388 | 28917481 | 478_203541_84133(ZNRF3)_2 | 0 | — |
| chr22 | 29048388 | 29048491 | 478_203549_84133(ZNRF3)_10 | 0 | — |
| chr13 | 28070490 | 28070612 | 461_60854_2322(FLT3)_3a | 0 | — |
| chr2 | 212124751 | 212124903 | 461_58773_2066(ERBB4)_2a | 0 | — |
| chr2 | 211704103 | 211704194 | 461_58782_2066(ERBB4)_11a | 0 | — |
| chr2 | 211623921 | 211624044 | 461_58790_2066(ERBB4)_19a | 0 | — |
| chr2 | 211387944 | 211387992 | 461_58798_2066(ERBB4)_27a | 0 | — |
| chr13 | 28037184 | 28037288 | 461_60862_2322(FLT3)_11a | 0 | — |
| chr13 | 28024860 | 28024943 | 461_60870_2322(FLT3)_19a | 0 | — |
| chr22 | 28883766 | 28884066 | 478_203540_84133(ZNRF3)_1 | 0 | — |
| chr22 | 28986622 | 28986639 | 478_203542_84133(ZNRF3)_3 | 0 | — |
| chr22 | 28987075 | 28987201 | 478_203543_84133(ZNRF3)_4 | 0 | — |
| chr4 | 1802913 | 1803064 | 461_60341_2261(FGFR3)_8a | 0 | — |
| chr4 | 1804329 | 1804520 | 461_60343_2261(FGFR3)_10a | 0 | — |
| chr4 | 1806828 | 1806934 | 461_60351_2261(FGFR3)_18a | 0 | — |
| chr4 | 1803691 | 1803836 | 461_60342_2261(FGFR3)_9a | 0 | — |
| chr4 | 1804823 | 1804969 | 461_60344_2261(FGFR3)_11a | 0 | — |
| chr19 | 2194514 | 2194577 | 431_55607_84444(DOT1L)_7 | 0 | — |
| chr19 | 2211098 | 2211212 | 431_55615_84444(DOT1L)_15 | 0 | — |
| chr19 | 2220107 | 2220222 | 431_55623_84444(DOT1L)_23 | 0 | — |
| chr19 | 1221948 | 1222006 | 475_170593_6794(STK11)_7a | 0 | — |
| chr19 | 1206913 | 1207203 | 475_170587_6794(STK11)_1a | 0 | — |
| chr19 | 1218416 | 1218500 | 475_170588_6794(STK11)_2a | 0 | — |
| chr2 | 211619176 | 211619275 | 461_58791_2066(ERBB4)_20a | 0 | — |
| chr2 | 211561902 | 211562088 | 461_58792_2066(ERBB4)_21a | 0 | — |
| chr2 | 211430944 | 211431100 | 461_58793_2066(ERBB4)_22a | 0 | — |
| chr2 | 211428407 | 211428483 | 461_58794_2066(ERBB4)_23a | 0 | — |
| chr2 | 211424154 | 211424301 | 461_58795_2066(ERBB4)_24a | 0 | — |
| chr2 | 211422006 | 211422104 | 461_58796_2066(ERBB4)_25a | 0 | — |
| chr2 | 211420440 | 211420611 | 461_58797_2066(ERBB4)_26a | 0 | — |
| chr2 | 211386852 | 211387150 | 461_58799_2066(ERBB4)_28a | 0 | — |
| chr2 | 211383614 | 211384060 | 461_58800_2066(ERBB4)_29a | 0 | — |
| chr12 | 56087794 | 56087913 | 461_58655_2065(ERBB3)_6a | 0 | — |
| chr12 | 56094098 | 56094189 | 461_58757_2065(ERBB3)_14a | 0 | — |
| chr12 | 56098499 | 56098575 | 461_58765_2065(ERBB3)_22a | 0 | — |
| chr19 | 1219323 | 1219413 | 475_170589_6794(STK11)_3a | 0 | — |
| chr19 | 1220372 | 1220505 | 475_170590_6794(STK11)_4a | 0 | — |
| chr19 | 1220580 | 1220717 | 475_170591_6794(STK11)_5a | 0 | — |
| chr19 | 1221212 | 1221340 | 475_170592_6794(STK11)_6a | 0 | — |
| chr19 | 1222984 | 1223172 | 475_170594_6794(STK11)_8a | 0 | — |
| chr19 | 1223637 | 1226446 | 475_170595_6794(STK11)_9a | 0 | — |
| chr19 | 1226453 | 1226663 | 475_170596_6794(STK11)_10a | 0 | — |
| chr19 | 1227592 | 1228082 | 475_170597_6794(STK11)_11a | 0 | — |
| chr20 | 37393894 | 37393993 | 475_169919_6714(SRC)_7a | 0 | — |
| chr12 | 56099647 | 56099745 | 461_58767_2065(ERBB3)_24a | 0 | — |
| chr12 | 56099837 | 56100029 | 461_58768_2065(ERBB3)_25a | 0 | — |
| chr12 | 56100173 | 56100245 | 461_58769_2065(ERBB3)_26a | 0 | — |
| chr12 | 56101060 | 56101361 | 461_58770_2065(ERBB3)_27a | 0 | — |
| chr12 | 56101528 | 56102055 | 461_58771_2065(ERBB3)_28a | 0 | — |
| chr20 | 37402748 | 37402880 | 475_169926_6714(SRC)_14a | 0 | — |
| chr20 | 37384153 | 37384403 | 475_169917_6714(SRC)_5a | 0 | — |
| chr20 | 37386074 | 37386174 | 475_169918_6714(SRC)_6a | 0 | — |
| chr20 | 37394173 | 37394277 | 475_169920_6714(SRC)_8a | 0 | — |
| chr20 | 37396161 | 37396311 | 475_169921_6714(SRC)_9a | 0 | — |
| chr20 | 37397698 | 37397854 | 475_169922_6714(SRC)_10a | 0 | — |
| chr20 | 37400114 | 37400294 | 475_169923_6714(SRC)_11a | 0 | — |
| chr20 | 37401601 | 37401678 | 475_169924_6714(SRC)_12a | 0 | — |
| chr20 | 37402434 | 37402588 | 475_169925_6714(SRC)_13a | 0 | — |
| chr20 | 37403170 | 37403379 | 475_169927_6714(SRC)_15a | 0 | — |
| chr15 | 66435026 | 66435237 | 465_99851_5604(MAP2K1)_3a | 0 | — |
| chr17 | 39700000 | 39700311 | 461_58728_2064(ERBB2)_6a | 0 | — |
| chr17 | 39712321 | 39712448 | 461_58736_2064(ERBB2)_14a | 0 | — |
| chr17 | 39723318 | 39723457 | 461_58744_2064(ERBB2)_22a | 0 | — |
| chr17 | 39726814 | 39727003 | 461_58752_2064(ERBB2)_30a | 0 | — |
| chr17 | 39699559 | 39699587 | 461_58727_2064(ERBB2)_5a | 0 | — |
| chr17 | 39706987 | 39707141 | 461_58729_2064(ERBB2)_7a | 0 | — |
| chr17 | 39708320 | 39708534 | 461_58730_2064(ERBB2)_8a | 0 | — |
| chr17 | 39709317 | 39709452 | 461_58731_2064(ERBB2)_9a | 0 | — |
| chr17 | 39709812 | 39709881 | 461_58732_2064(ERBB2)_10a | 0 | — |
| chr17 | 39710085 | 39710201 | 461_58733_2064(ERBB2)_11a | 0 | — |
| chr17 | 39710339 | 39710481 | 461_58734_2064(ERBB2)_12a | 0 | — |
| chr17 | 39711927 | 39712047 | 461_58735_2064(ERBB2)_13a | 0 | — |
| chr17 | 39715285 | 39715359 | 461_58737_2064(ERBB2)_15a | 0 | — |
| chr17 | 39715445 | 39715536 | 461_58738_2064(ERBB2)_16a | 0 | — |
| chr17 | 39715739 | 39715939 | 461_58739_2064(ERBB2)_17a | 0 | — |
| chr17 | 39716300 | 39716433 | 461_58740_2064(ERBB2)_18a | 0 | — |
| chr17 | 39716514 | 39716605 | 461_58741_2064(ERBB2)_19a | 0 | — |
| chr17 | 39717319 | 39717480 | 461_58742_2064(ERBB2)_20a | 0 | — |
| chr17 | 39719786 | 39719834 | 461_58743_2064(ERBB2)_21a | 0 | — |
| chr17 | 39723537 | 39723660 | 461_58745_2064(ERBB2)_23a | 0 | — |
| chr17 | 39723911 | 39724010 | 461_58746_2064(ERBB2)_24a | 0 | — |
| chr17 | 39724725 | 39724911 | 461_58747_2064(ERBB2)_25a | 0 | — |
| chr17 | 39725048 | 39725204 | 461_58748_2064(ERBB2)_26a | 0 | — |
| chr17 | 39725326 | 39725402 | 461_58749_2064(ERBB2)_27a | 0 | — |
| chr17 | 39725706 | 39725853 | 461_58750_2064(ERBB2)_28a | 0 | — |
| chr17 | 39726561 | 39726659 | 461_58751_2064(ERBB2)_29a | 0 | — |
| chr17 | 39727294 | 39727547 | 461_58753_2064(ERBB2)_31a | 0 | — |
| chr17 | 39727688 | 39728044 | 461_58754_2064(ERBB2)_32a | 0 | — |
| chr15 | 66490501 | 66490615 | 465_99860_5604(MAP2K1)_12a | 0 | — |
| chr15 | 66387347 | 66387427 | 465_99849_5604(MAP2K1)_1a | 0 | — |
| chr15 | 66401862 | 66401947 | 465_99850_5604(MAP2K1)_2a | 0 | — |
| chr15 | 66436745 | 66436892 | 465_99852_5604(MAP2K1)_4a | 0 | — |
| chr15 | 66443279 | 66443357 | 465_99853_5604(MAP2K1)_5a | 0 | — |
| chr1 | 113585386 | 113585596 | 465_103739_260425(MAGI3)_4 | 0 | — |
| chr1 | 113643742 | 113643774 | 465_103747_260425(MAGI3)_12 | 0 | — |
| chr1 | 113673321 | 113673465 | 465_103755_260425(MAGI3)_20 | 0 | — |
| chr1 | 113391033 | 113391349 | 465_103736_260425(MAGI3)_1 | 0 | — |
| chr1 | 113549514 | 113549631 | 465_103737_260425(MAGI3)_2 | 0 | — |
| chr1 | 113580541 | 113580661 | 465_103738_260425(MAGI3)_3 | 0 | — |
| chr1 | 113590483 | 113590658 | 465_103740_260425(MAGI3)_5 | 0 | — |
| chr17 | 31356459 | 31356582 | 469_118625_4763(NF1)_52a | 0 | — |
| chr17 | 31352256 | 31352414 | 469_118624_4763(NF1)_51a | 0 | — |
| chr17 | 31350182 | 31350318 | 469_118623_4763(NF1)_50a | 0 | — |
| chr1 | 26774328 | 26775220 | 426_4235_8289(ARID1A)_18a | 0 | — |
| chr1 | 26775576 | 26775707 | 426_4236_8289(ARID1A)_19a | 0 | — |
| chr1 | 26779022 | 26780756 | 426_4237_8289(ARID1A)_20a | 0 | — |
| chr5 | 112801278 | 112801383 | 426_2913_324(APC)_11a | 0 | — |
| chr5 | 112837552 | 112844126 | 426_2921_324(APC)_19a | 0 | — |
| chr5 | 112707717 | 112707882 | 426_2903_324(APC)_1a | 0 | — |
| chr5 | 112737858 | 112737925 | 426_2904_324(APC)_2a | 0 | — |
| chr5 | 112738331 | 112738460 | 426_2905_324(APC)_3a | 0 | — |
| chr5 | 112754872 | 112755025 | 426_2906_324(APC)_4a | 0 | — |
| chr5 | 112755074 | 112755094 | 426_2907_324(APC)_5a | 0 | — |
| chr5 | 112766325 | 112766410 | 426_2908_324(APC)_6a | 0 | — |
| chr5 | 112767188 | 112767390 | 426_2909_324(APC)_7a | 0 | — |
| chr5 | 112775628 | 112775737 | 426_2910_324(APC)_8a | 0 | — |
| chr5 | 112780789 | 112780903 | 426_2911_324(APC)_9a | 0 | — |
| chr5 | 112792445 | 112792529 | 426_2912_324(APC)_10a | 0 | — |
| chr5 | 112815494 | 112815593 | 426_2914_324(APC)_12a | 0 | — |
| chr5 | 112818965 | 112819344 | 426_2915_324(APC)_13a | 0 | — |
| chr5 | 112821895 | 112821991 | 426_2916_324(APC)_14a | 0 | — |
| chr5 | 112827107 | 112827247 | 426_2917_324(APC)_15a | 0 | — |
| chr5 | 112827928 | 112828006 | 426_2918_324(APC)_16a | 0 | — |
| chr1 | 113594480 | 113594560 | 465_103741_260425(MAGI3)_6 | 0 | — |
| chr16 | 68801669 | 68801893 | 428_25422_999(CDH1)_3 | 0 | — |
| chr16 | 68815514 | 68815759 | 428_25429_999(CDH1)_10 | 0 | — |
| chr16 | 68737415 | 68737463 | 428_25420_999(CDH1)_1 | 0 | — |
| chr16 | 68738296 | 68738411 | 428_25421_999(CDH1)_2 | 0 | — |
| chr16 | 68808423 | 68808567 | 428_25423_999(CDH1)_4 | 0 | — |
| chr1 | 113614600 | 113614658 | 465_103742_260425(MAGI3)_70 | 0 | — |
| chr1 | 113618531 | 113618606 | 465_103743_260425(MAGI3)_80 | 0 | — |
| chr1 | 113619735 | 113619830 | 465_103744_260425(MAGI3)_90 | 0 | — |
| chr1 | 113622805 | 113622994 | 465_103745_260425(MAGI3)_10 | 0 | — |
| chr1 | 113641910 | 113642516 | 465_103746_260425(MAGI3)_11 | 0 | — |
| chr1 | 113646485 | 113646642 | 465_103748_260425(MAGI3)_13 | 0 | — |
| chr1 | 113649236 | 113649328 | 465_103749_260425(MAGI3)_14 | 0 | — |
| chr1 | 113651013 | 113651206 | 465_103750_260425(MAGI3)_15 | 0 | — |
| chr1 | 113653829 | 113654018 | 465_103751_260425(MAGI3)_16 | 0 | — |
| chr1 | 113659079 | 113659265 | 465_103752_260425(MAGI3)_17 | 0 | — |
| chr1 | 113671733 | 113671836 | 465_103753_260425(MAGI3)_18 | 0 | — |
| chr1 | 113672614 | 113672741 | 465_103754_260425(MAGI3)_19 | 0 | — |
| chr1 | 113681197 | 113681336 | 465_103756_260425(MAGI3)_21 | 0 | — |
| chr1 | 113682211 | 113684014 | 465_103757_260425(MAGI3)_22 | 0 | — |
| chr16 | 68808692 | 68808848 | 428_25424_999(CDH1)_5 | 0 | — |
| chr16 | 68810196 | 68810341 | 428_25425_999(CDH1)_6 | 0 | — |
| chr16 | 68811683 | 68811859 | 428_25426_999(CDH1)_7 | 0 | — |
| chr16 | 68812134 | 68812263 | 428_25427_999(CDH1)_8 | 0 | — |
| chr16 | 68813312 | 68813495 | 428_25428_999(CDH1)_9 | 0 | — |
| chr7 | 152315137 | 152315338 | 464_94967_58508(KMT2C)_4 | 0 | — |
| chr7 | 152249875 | 152249953 | 464_94976_58508(KMT2C)_13 | 0 | — |
| chr7 | 152222572 | 152222682 | 464_94984_58508(KMT2C)_21 | 0 | — |
| chr7 | 152194439 | 152194568 | 464_94993_58508(KMT2C)_30 | 0 | — |
| chr7 | 152171263 | 152171342 | 464_95004_58508(KMT2C)_41 | 0 | — |
| chr7 | 152154009 | 152154146 | 464_95013_58508(KMT2C)_50 | 0 | — |
| chr7 | 152139674 | 152139791 | 464_95021_58508(KMT2C)_58 | 0 | — |
| chr16 | 68819279 | 68819425 | 428_25430_999(CDH1)_11 | 0 | — |
| chr7 | 152180710 | 152182594 | 464_95000_58508(KMT2C)_37 | 0 | — |
| chr7 | 152179833 | 152180126 | 464_95001_58508(KMT2C)_38 | 0 | — |
| chr7 | 152176190 | 152178010 | 464_95002_58508(KMT2C)_39 | 0 | — |
| chr7 | 152174130 | 152174242 | 464_95003_58508(KMT2C)_40 | 0 | — |
| chr7 | 152169185 | 152169249 | 464_95005_58508(KMT2C)_42 | 0 | — |
| chr7 | 152167145 | 152167378 | 464_95006_58508(KMT2C)_43 | 0 | — |
| chr7 | 152162116 | 152163826 | 464_95007_58508(KMT2C)_44 | 0 | — |
| chr7 | 152158856 | 152159072 | 464_95008_58508(KMT2C)_45 | 0 | — |
| chr7 | 152157760 | 152157925 | 464_95009_58508(KMT2C)_46 | 0 | — |
| chr7 | 152156204 | 152156346 | 464_95010_58508(KMT2C)_47 | 0 | — |
| chr7 | 152155909 | 152156057 | 464_95011_58508(KMT2C)_48 | 0 | — |
| chr7 | 152154266 | 152154445 | 464_95012_58508(KMT2C)_49 | 0 | — |
| chr7 | 152152704 | 152152954 | 464_95014_58508(KMT2C)_51 | 0 | — |
| chr7 | 152151441 | 152151581 | 464_95015_58508(KMT2C)_52 | 0 | — |
| chr7 | 152150899 | 152151007 | 464_95016_58508(KMT2C)_53 | 0 | — |
| chr7 | 152148032 | 152149152 | 464_95017_58508(KMT2C)_54 | 0 | — |
| chr7 | 152146598 | 152146735 | 464_95018_58508(KMT2C)_55 | 0 | — |
| chr7 | 152145152 | 152145295 | 464_95019_58508(KMT2C)_56 | 0 | — |
| chr7 | 152144712 | 152144881 | 464_95020_58508(KMT2C)_57 | 0 | — |
| chr7 | 152435625 | 152435786 | 464_94964_58508(KMT2C)_1 | 0 | — |
| chr7 | 152358586 | 152358675 | 464_94965_58508(KMT2C)_2 | 0 | — |
| chr7 | 152330600 | 152330742 | 464_94966_58508(KMT2C)_3 | 0 | — |
| chr7 | 152311797 | 152311946 | 464_94968_58508(KMT2C)_5 | 0 | — |
| chr7 | 152309965 | 152310075 | 464_94969_58508(KMT2C)_6 | 0 | — |
| chr7 | 152273704 | 152273867 | 464_94970_58508(KMT2C)_7 | 0 | — |
| chr7 | 152265037 | 152265209 | 464_94971_58508(KMT2C)_8 | 0 | — |
| chr7 | 152263015 | 152263130 | 464_94972_58508(KMT2C)_9 | 0 | — |
| chr7 | 152252545 | 152252715 | 464_94973_58508(KMT2C)_1 | 0 | — |
| chr16 | 68822000 | 68822225 | 428_25431_999(CDH1)_12 | 0 | — |
| chr7 | 152251938 | 152252090 | 464_94974_58508(KMT2C)_11 | 0 | — |
| chr7 | 152250852 | 152250966 | 464_94975_58508(KMT2C)_12 | 0 | — |
| chr7 | 152247901 | 152248620 | 464_94977_58508(KMT2C)_14 | 0 | — |
| chr7 | 152238706 | 152238826 | 464_94978_58508(KMT2C)_15 | 0 | — |
| chr7 | 152235816 | 152235933 | 464_94979_58508(KMT2C)_16 | 0 | — |
| chr7 | 152230219 | 152230321 | 464_94980_58508(KMT2C)_17 | 0 | — |
| chr7 | 152229922 | 152230027 | 464_94981_58508(KMT2C)_18 | 0 | — |
| chr5 | 112828855 | 112828972 | 426_2919_324(APC)_17a | 0 | — |
| chr5 | 112834950 | 112835165 | 426_2920_324(APC)_18a | 0 | — |
| chr14 | 104777568 | 104777740 | 426_1772_207(AKT1)_6a | 0 | — |
| chr14 | 104776658 | 104776770 | 426_1773_207(AKT1)_7a | 0 | — |
| chr14 | 104775075 | 104775207 | 426_1775_207(AKT1)_9a | 0 | — |
| chr14 | 104774937 | 104775003 | 426_1776_207(AKT1)_10a | 0 | — |
| chr14 | 104773911 | 104773980 | 426_1777_207(AKT1)_11a | 0 | — |
| chr14 | 104773454 | 104773580 | 426_1778_207(AKT1)_12a | 0 | — |
| chr14 | 104773250 | 104773379 | 426_1779_207(AKT1)_13a | 0 | — |
| chr14 | 104772877 | 104773092 | 426_1780_207(AKT1)_14a | 0 | — |
| chr14 | 104772364 | 104772452 | 426_1781_207(AKT1)_15a | 0 | — |
| chr14 | 104770340 | 104770420 | 426_1783_207(AKT1)_17a | 0 | — |
| chr9 | 130714319 | 130714455 | 426_351_25(ABL1)_1 | 0 | — |
| chr9 | 130835443 | 130835525 | 426_352_25(ABL1)_2 | 0 | — |
| chr7 | 152224434 | 152224616 | 464_94982_58508(KMT2C)_19 | 0 | — |
| chr1 | 11258484 | 11258593 | 466_105882_2475(MTOR)_3a | 0 | — |
| chr1 | 11243113 | 11243300 | 466_105889_2475(MTOR)_10a | 0 | — |
| chr1 | 11231299 | 11231434 | 466_105897_2475(MTOR)_18a | 0 | — |
| chr1 | 11209311 | 11209458 | 466_105905_2475(MTOR)_26a | 0 | — |
| chr1 | 11146675 | 11146791 | 466_105912_2475(MTOR)_33a | 0 | — |
| chr1 | 11129737 | 11129838 | 466_105920_2475(MTOR)_41a | 0 | — |
| chr1 | 11121978 | 11122126 | 466_105928_2475(MTOR)_49a | 0 | — |
| chr1 | 11109648 | 11109729 | 466_105935_2475(MTOR)_56a | 0 | — |
| chr7 | 152139185 | 152139259 | 464_95022_58508(KMT2C)_59 | 0 | — |
| chr7 | 140801411 | 140801560 | 427_15635_673(BRAF)_6 | 0 | — |
| chr7 | 140800361 | 140800481 | 427_15636_673(BRAF)_7 | 0 | — |
| chr16 | 68823398 | 68823626 | 428_25432_999(CDH1)_13 | 0 | — |
| chr16 | 68828173 | 68828304 | 428_25433_999(CDH1)_14 | 0 | — |
| chr16 | 68829653 | 68829797 | 428_25434_999(CDH1)_15 | 0 | — |
| chr16 | 68833289 | 68833499 | 428_25435_999(CDH1)_16 | 0 | — |
| chr7 | 152138795 | 152138904 | 464_95023_58508(KMT2C)_60 | 0 | — |
| chr7 | 152136831 | 152136924 | 464_95024_58508(KMT2C)_61 | 0 | — |
| chr9 | 130883968 | 130885683 | 426_362_25(ABL1)_12 | 0 | — |
| chr9 | 130862762 | 130863035 | 426_355_25(ABL1)_5 | 0 | — |
| chr13 | 32316460 | 32316527 | 427_15650_675(BRCA2)_2 | 0 | — |
| chr13 | 32319076 | 32319325 | 427_15651_675(BRCA2)_3 | 0 | — |
| chr13 | 32325075 | 32325184 | 427_15652_675(BRCA2)_4 | 0 | — |
| chr13 | 32326241 | 32326282 | 427_15654_675(BRCA2)_6 | 0 | — |
| chr13 | 32326498 | 32326613 | 427_15655_675(BRCA2)_7 | 0 | — |
| chr13 | 32329442 | 32329492 | 427_15656_675(BRCA2)_8 | 0 | — |
| chr13 | 32330918 | 32331030 | 427_15657_675(BRCA2)_9 | 0 | — |
| chr13 | 32332271 | 32333387 | 427_15658_675(BRCA2)_10 | 0 | — |
| chr13 | 32336264 | 32341196 | 427_15659_675(BRCA2)_11 | 0 | — |
| chr13 | 32344557 | 32344653 | 427_15660_675(BRCA2)_12 | 0 | — |
| chr13 | 32354897 | 32355325 | 427_15662_675(BRCA2)_14 | 0 | — |
| chr13 | 32356427 | 32356609 | 427_15663_675(BRCA2)_15 | 0 | — |
| chr4 | 54698283 | 54698565 | 464_88968_3815(KIT)_3 | 0 | — |
| chr4 | 54727217 | 54727324 | 464_88975_3815(KIT)_10 | 0 | — |
| chr4 | 54736497 | 54736609 | 464_88983_3815(KIT)_18 | 0 | — |
| chr4 | 54658014 | 54658081 | 464_88966_3815(KIT)_1 | 0 | — |
| chr4 | 54695511 | 54695781 | 464_88967_3815(KIT)_2 | 0 | — |
| chr4 | 54699629 | 54699766 | 464_88969_3815(KIT)_4 | 0 | — |
| chr6 | 152098731 | 152098966 | 461_58886_2099(ESR1)_12 | 0 | — |
| chr6 | 151807912 | 151808364 | 461_58879_2099(ESR1)_5 | 0 | — |
| chr6 | 151842596 | 151842793 | 461_58880_2099(ESR1)_6 | 0 | — |
| chr6 | 151880654 | 151880771 | 461_58881_2099(ESR1)_7 | 0 | — |
| chr6 | 151944172 | 151944508 | 461_58882_2099(ESR1)_8 | 0 | — |
| chr6 | 152011655 | 152011794 | 461_58883_2099(ESR1)_9 | 0 | — |
| chr4 | 54703720 | 54703892 | 464_88970_3815(KIT)_5 | 0 | — |
| chr4 | 54707097 | 54707287 | 464_88971_3815(KIT)_6 | 0 | — |
| chr4 | 54709423 | 54709539 | 464_88972_3815(KIT)_7 | 0 | — |
| chr4 | 54723583 | 54723698 | 464_88973_3815(KIT)_8 | 0 | — |
| chr4 | 54725856 | 54726050 | 464_88974_3815(KIT)_9 | 0 | — |
| chr4 | 54727415 | 54727542 | 464_88976_3815(KIT)_11 | 0 | — |
| chr4 | 54727822 | 54727927 | 464_88977_3815(KIT)_12 | 0 | — |
| chr4 | 54728010 | 54728121 | 464_88978_3815(KIT)_13 | 0 | — |
| chr4 | 54729334 | 54729485 | 464_88979_3815(KIT)_14 | 0 | — |
| chr4 | 54731327 | 54731419 | 464_88980_3815(KIT)_15 | 0 | — |
| chr4 | 54731870 | 54731998 | 464_88981_3815(KIT)_16 | 0 | — |
| chr4 | 54733069 | 54733192 | 464_88982_3815(KIT)_17 | 0 | — |
| chr7 | 140787547 | 140787584 | 427_15638_673(BRAF)_9 | 0 | — |
| chr7 | 140783020 | 140783157 | 427_15639_673(BRAF)_10 | 0 | — |
| chr7 | 140781575 | 140781693 | 427_15640_673(BRAF)_11 | 0 | — |
| chr7 | 140777990 | 140778075 | 427_15641_673(BRAF)_12 | 0 | — |
| chr7 | 140776911 | 140777088 | 427_15642_673(BRAF)_13 | 0 | — |
| chr7 | 140754186 | 140754233 | 427_15643_673(BRAF)_14 | 0 | — |
| chr7 | 140753274 | 140753393 | 427_15644_673(BRAF)_15 | 0 | — |
| chr7 | 140747365 | 140747447 | 427_15646_673(BRAF)_17 | 0 | — |
| chr7 | 140739811 | 140739946 | 427_15647_673(BRAF)_18 | 0 | — |
| chr7 | 140734011 | 140734770 | 427_15648_673(BRAF)_19 | 0 | — |
| chr4 | 54736720 | 54736820 | 464_88984_3815(KIT)_19 | 0 | — |
| chr7 | 140719333 | 140726516 | 427_15649_673(BRAF)_20 | 0 | — |
| chr9 | 130854063 | 130854237 | 426_353_25(ABL1)_3 | 0 | — |
| chr9 | 130854800 | 130855096 | 426_354_25(ABL1)_4 | 0 | — |
| chr1 | 26696403 | 26697540 | 426_4218_8289(ARID1A)_1a | 0 | — |
| chr1 | 26729650 | 26729863 | 426_4219_8289(ARID1A)_2a | 0 | — |
| chr1 | 26731151 | 26731604 | 426_4220_8289(ARID1A)_3a | 0 | — |
| chr1 | 26732675 | 26732792 | 426_4221_8289(ARID1A)_4a | 0 | — |
| chr4 | 54737174 | 54737280 | 464_88985_3815(KIT)_20 | 0 | — |
| chr1 | 26760855 | 26761096 | 426_4222_8289(ARID1A)_5a | 0 | — |
| chr1 | 26761383 | 26761473 | 426_4223_8289(ARID1A)_6a | 0 | — |
| chr5 | 56884663 | 56884826 | 465_99634_4214(MAP3K1)_18a | 0 | — |
| chr5 | 56887377 | 56887520 | 465_99636_4214(MAP3K1)_20a | 0 | — |
| chr5 | 56888225 | 56888357 | 465_99637_4214(MAP3K1)_21a | 0 | — |
| chr5 | 56893530 | 56893680 | 465_99638_4214(MAP3K1 )_22a | 0 | — |
| chr17 | 12110374 | 12110426 | 465_99954_6416(MAP2K4)_8 | 0 | — |
| chr1 | 26762972 | 26763285 | 426_4225_8289(ARID1A)_8a | 0 | — |
| chr1 | 26766220 | 26766366 | 426_4226_8289(ARID1A)_9a | 0 | — |
| chr13 | 32357741 | 32357929 | 427_15664_675(BRCA2)_16 | 0 | — |
| chr2 | 29296887 | 29297057 | 426_1978_238(ALK)_9 | 0 | — |
| chr2 | 29275401 | 29275496 | 426_1979_238(ALK)_10 | 0 | — |
| chr2 | 29275098 | 29275227 | 426_1980_238(ALK)_11 | 0 | — |
| chr2 | 29251104 | 29251267 | 426_1981_238(ALK)_12 | 0 | — |
| chr2 | 29239679 | 29239830 | 426_1982_238(ALK)_13 | 0 | — |
| chr2 | 29232303 | 29232448 | 426_1984_238(ALK)_15 | 0 | — |
| chr2 | 29228883 | 29229066 | 426_1985_238(ALK)_16 | 0 | — |
| chr2 | 29227573 | 29227672 | 426_1986_238(ALK)_17 | 0 | — |
| chr2 | 29226921 | 29227074 | 426_1987_238(ALK)_18 | 0 | — |
| chr2 | 29225460 | 29225565 | 426_1988_238(ALK)_19 | 0 | — |
| chr2 | 29223341 | 29223528 | 426_1989_238(ALK)_20 | 0 | — |
| chr2 | 29222516 | 29222607 | 426_1990_238(ALK)_21 | 0 | — |
| chr2 | 29222343 | 29222408 | 426_1991_238(ALK)_22 | 0 | — |
| chr2 | 29213983 | 29214081 | 426_1993_238(ALK)_24 | 0 | — |
| chr2 | 29209785 | 29209878 | 426_1994_238(ALK)_25 | 0 | — |
| chr2 | 29207170 | 29207272 | 426_1995_238(ALK)_26 | 0 | — |
| chr2 | 29197541 | 29197676 | 426_1996_238(ALK)_27 | 0 | — |
| chr3 | 142559250 | 142559441 | 427_14859_545(ATR)_7 | 0 | — |
| chr3 | 142550131 | 142550302 | 427_14866_545(ATR)_14 | 0 | — |
| chr3 | 142538481 | 142538625 | 427_14871_545(ATR)_19 | 0 | — |
| chr3 | 142513500 | 142513638 | 427_14878_545(ATR)_26 | 0 | — |
| chr3 | 142496360 | 142496520 | 427_14886_545(ATR)_34 | 0 | — |
| chr3 | 142461939 | 142462090 | 427_14894_545(ATR)_42 | 0 | — |
| chr2 | 29196769 | 29196860 | 426_1997_238(ALK)_28 | 0 | — |
| chr2 | 29193223 | 29193922 | 426_1998_238(ALK)_29 | 0 | — |
| chr14 | 104775651 | 104775799 | 426_1774_207(AKT1)_8a | 0 | — |
| chr14 | 104770744 | 104770847 | 426_1782_207(AKT1)_16a | 0 | — |
| chr14 | 104792597 | 104792643 | 426_1770_207(AKT1)_4a | 0 | — |
| chr14 | 104780087 | 104780216 | 426_1771_207(AKT1)_5a | 0 | — |
| chr4 | 54738428 | 54738557 | 464_88986_3815(KIT)_21 | 0 | — |
| chr2 | 29328349 | 29328481 | 426_1975_238(ALK)_6 | 0 | — |
| chr2 | 29233564 | 29233696 | 426_1983_238(ALK)_14 | 0 | — |
| chr2 | 29220705 | 29220835 | 426_1992_238(ALK)_23 | 0 | — |
| chr2 | 29919992 | 29920659 | 426_1970_238(ALK)_1 | 0 | — |
| chr2 | 29717577 | 29717697 | 426_1971_238(ALK)_2 | 0 | — |
| chr2 | 29694849 | 29695014 | 426_1972_238(ALK)_3 | 0 | — |
| chr2 | 29531914 | 29532116 | 426_1973_238(ALK)_4 | 0 | — |
| chr2 | 29383731 | 29383859 | 426_1974_238(ALK)_5 | 0 | — |
| chr2 | 29320750 | 29320882 | 426_1976_238(ALK)_7 | 0 | — |
| chr1 | 26766456 | 26766566 | 426_4227_8289(ARID1A)_10a | 0 | — |
| chr1 | 26767789 | 26767999 | 426_4228_8289(ARID1A)_11a | 0 | — |
| chr9 | 130872128 | 130872213 | 426_356_25(ABL1)_6 | 0 | — |
| chr9 | 130872859 | 130873037 | 426_357_25(ABL1)_7 | 0 | — |
| chr9 | 130874867 | 130875052 | 426_358_25(ABL1)_8 | 0 | — |
| chr9 | 130878414 | 130878567 | 426_359_25(ABL1)_9 | 0 | — |
| chr9 | 130880067 | 130880157 | 426_360_25(ABL1)_10 | 0 | — |
| chr9 | 130880499 | 130880664 | 426_361_25(ABL1)_11 | 0 | — |
| chr13 | 32362522 | 32362693 | 427_15665_675(BRCA2)_17 | 0 | — |
| chr13 | 32363178 | 32363533 | 427_15666_675(BRCA2)_18 | 0 | — |
| chr13 | 32370401 | 32370557 | 427_15667_675(BRCA2)_19 | 0 | — |
| chr13 | 32370955 | 32371100 | 427_15668_675(BRCA2)_20 | 0 | — |
| chr18 | 51078255 | 51078467 | 474_158727_4089(SMAD4)_13 | 0 | — |
| chr18 | 51077306 | 51077419 | 474_158726_4089(SMAD4)_12 | 0 | — |
| chr18 | 51067018 | 51067187 | 474_158724_4089(SMAD4)_10 | 0 | — |
| chr18 | 51065422 | 51065606 | 474_158723_4089(SMAD4)_9 | 0 | — |
| chr18 | 51059865 | 51059916 | 474_158722_4089(SMAD4)_8 | 0 | — |
| chr18 | 51058339 | 51058456 | 474_158721_4089(SMAD4)_7 | 0 | — |
| chr18 | 51058124 | 51058244 | 474_158720_4089(SMAD4)_6 | 0 | — |
| chr18 | 51054780 | 51054993 | 474_158719_4089(SMAD4)_5 | 0 | — |
| chr18 | 51048685 | 51048860 | 474_158717_4089(SMAD4)_3 | 0 | — |
| chr18 | 51047046 | 51047295 | 474_158716_4089(SMAD4)_2 | 0 | — |
| chr18 | 51076637 | 51076776 | 474_158725_4089(SMAD4)_11 | 0 | — |
| chr9 | 77922160 | 77922345 | 462_70176_2776(GNAQ)_2 | 0 | — |
| chr13 | 32379316 | 32379515 | 427_15670_675(BRCA2)_22 | 0 | — |
| chr11 | 108235669 | 108235834 | 427_14214_472(ATM)_8 | 0 | — |
| chr6 | 152060990 | 152061124 | 461_58884_2099(ESR1)_10 | 0 | — |
| chr6 | 152094384 | 152094568 | 461_58885_2099(ESR1)_11 | 0 | — |
| chr13 | 32379749 | 32379913 | 427_15671_675(BRCA2)_23 | 0 | — |
| chr13 | 32380006 | 32380145 | 427_15672_675(BRCA2)_24 | 0 | — |
| chr13 | 32394688 | 32394933 | 427_15673_675(BRCA2)_25 | 0 | — |
| chr13 | 32396897 | 32397044 | 427_15674_675(BRCA2)_26 | 0 | — |
| chr13 | 32398161 | 32398770 | 427_15675_675(BRCA2)_27 | 0 | — |
| chr2 | 29318303 | 29318404 | 426_1977_238(ALK)_8 | 0 | — |
| chr1 | 26771118 | 26771326 | 426_4229_8289(ARID1A)_12a | 0 | — |
| chr1 | 26772499 | 26772632 | 426_4230_8289(ARID1A)_13a | 0 | — |
| chr1 | 26772811 | 26772987 | 426_4231_8289(ARID1A)_14a | 0 | — |
| chr1 | 26773579 | 26773717 | 426_4233_8289(ARID1A)_16a | 0 | — |
| chr1 | 26773801 | 26773898 | 426_4234_8289(ARID1A)_17a | 0 | — |
| chr13 | 28100467 | 28100510 | 461_60853_2322(FLT3)_2a | 0 | — |
| chr13 | 28061866 | 28062069 | 461_60855_2322(FLT3)_4a | 0 | — |
| chr13 | 28057346 | 28057462 | 461_60856_2322(FLT3)_5a | 0 | — |
| chr13 | 28052544 | 28052674 | 461_60857_2322(FLT3)_6a | 0 | — |
| chr13 | 28050094 | 28050222 | 461_60858_2322(FLT3)_7a | 0 | — |
| chr13 | 28049634 | 28049774 | 461_60859_2322(FLT3)_8a | 0 | — |
| chr13 | 28049383 | 28049537 | 461_60860_2322(FLT3)_9a | 0 | — |
| chr13 | 28048274 | 28048443 | 461_60861_2322(FLT3)_10a | 0 | — |
| chr13 | 28035934 | 28036043 | 461_60863_2322(FLT3)_12a | 0 | — |
| chr13 | 28035494 | 28035673 | 461_60864_2322(FLT3)_13a | 0 | — |
| chr13 | 28034300 | 28034407 | 461_60865_2322(FLT3)_14a | 0 | — |
| chr13 | 28034081 | 28034214 | 461_60866_2322(FLT3)_15a | 0 | — |
| chr13 | 28033886 | 28033991 | 461_60867_2322(FLT3)_16a | 0 | — |
| chr13 | 28028177 | 28028288 | 461_60868_2322(FLT3)_17a | 0 | — |
| chr13 | 28027087 | 28027241 | 461_60869_2322(FLT3)_18a | 0 | — |
| chr13 | 28023349 | 28023477 | 461_60871_2322(FLT3)_20a | 0 | — |
| chr13 | 28018466 | 28018589 | 461_60872_2322(FLT3)_21a | 0 | — |
| chr13 | 28015589 | 28015701 | 461_60873_2322(FLT3)_22a | 0 | — |
| chr13 | 28015156 | 28015256 | 461_60874_2322(FLT3)_23a | 0 | — |
| chr13 | 28014451 | 28014557 | 461_60875_2322(FLT3)_24a | 0 | — |
| chr17 | 43124016 | 43124096 | 427_15607_672(BRCA1)_2 | 0 | — |
| chr17 | 43115725 | 43115779 | 427_15608_672(BRCA1)_3 | 0 | — |
| chr17 | 43106455 | 43106533 | 427_15609_672(BRCA1)_4 | 0 | — |
| chr17 | 43104867 | 43104956 | 427_15610_672(BRCA1)_5 | 0 | — |
| chr17 | 43104121 | 43104261 | 427_15611_672(BRCA1)_6 | 0 | — |
| chr17 | 43099774 | 43099880 | 427_15612_672(BRCA1)_7 | 0 | — |
| chr17 | 43095845 | 43095922 | 427_15614_672(BRCA1)_9 | 0 | — |
| chr17 | 43091434 | 43094860 | 427_15615_672(BRCA1)_10 | 0 | — |
| chr17 | 43090943 | 43091032 | 427_15616_672(BRCA1)_11 | 0 | — |
| chr17 | 43082403 | 43082575 | 427_15617_672(BRCA1)_12 | 0 | — |
| chr17 | 43079333 | 43079399 | 427_15618_672(BRCA1)_13 | 0 | — |
| chr17 | 43076487 | 43076614 | 427_15619_672(BRCA1)_14 | 0 | — |
| chr17 | 43074330 | 43074521 | 427_15620_672(BRCA1)_15 | 0 | — |
| chr17 | 43067607 | 43067695 | 427_15622_672(BRCA1)_17 | 0 | — |
| chr17 | 43063873 | 43063951 | 427_15623_672(BRCA1)_18 | 0 | — |
| chr17 | 43063332 | 43063373 | 427_15624_672(BRCA1)_19 | 0 | — |
| chr17 | 43057051 | 43057135 | 427_15625_672(BRCA1)_20 | 0 | — |
| chr17 | 43051062 | 43051117 | 427_15626_672(BRCA1)_21 | 0 | — |
| chr17 | 43049120 | 43049194 | 427_15627_672(BRCA1)_22 | 0 | — |
| chr17 | 43047642 | 43047703 | 427_15628_672(BRCA1)_23 | 0 | — |
| chr7 | 140924565 | 140924756 | 427_15630_673(BRAF)_1 | 0 | — |
| chr7 | 140850110 | 140850212 | 427_15631_673(BRAF)_2 | 0 | — |
| chr7 | 140834608 | 140834872 | 427_15632_673(BRAF)_3 | 0 | — |
| chr7 | 140808891 | 140808995 | 427_15633_673(BRAF)_4 | 0 | — |
| chr7 | 140807959 | 140808062 | 427_15634_673(BRAF)_5 | 0 | — |
| chr13 | 28004051 | 28004174 | 461_60876_2322(FLT3)_25a | 0 | — |
| chr2 | 211657753 | 211657828 | 461_58788_2066(ERBB4)_17a | 0 | — |
| chr11 | 108365081 | 108365218 | 427_14272_472(ATM)_66 | 0 | — |
| chr11 | 108365324 | 108365508 | 427_14273_472(ATM)_67 | 0 | — |
| chr2 | 211630461 | 211630594 | 461_58789_2066(ERBB4)_18a | 0 | — |
| chr12 | 56080300 | 56080382 | 461_58650_2065(ERBB3)_1a | 0 | — |
| chr12 | 56083750 | 56083902 | 461_58651_2065(ERBB3)_2a | 0 | — |
| chr12 | 56084994 | 56085617 | 461_58652_2065(ERBB3)_3a | 0 | — |
| chr12 | 56086530 | 56086656 | 461_58653_2065(ERBB3)_4a | 0 | — |
| chr12 | 56087576 | 56087642 | 461_58654_2065(ERBB3)_5a | 0 | — |
| chr12 | 56088020 | 56088162 | 461_58656_2065(ERBB3)_7a | 0 | — |
| chr12 | 56088542 | 56088656 | 461_58657_2065(ERBB3)_8a | 0 | — |
| chr12 | 56088747 | 56088868 | 461_58658_2065(ERBB3)_9a | 0 | — |
| chr12 | 56092746 | 56092820 | 461_58659_2065(ERBB3)_10a | 0 | — |
| chr12 | 56092985 | 56093076 | 461_58660_2065(ERBB3)_11a | 0 | — |
| chr12 | 56093344 | 56093550 | 461_58755_2065(ERBB3)_12a | 0 | — |
| chr7 | 55019277 | 55019365 | 431_48541_1956(EGFR)_1 | 0 | — |
| chr7 | 55109722 | 55109958 | 431_48542_1956(EGFR)_2 | 0 | — |
| chr7 | 55142285 | 55142437 | 431_48543_1956(EGFR)_3 | 0 | — |
| chr7 | 55146605 | 55146740 | 431_48545_1956(EGFR)_5 | 0 | — |
| chr7 | 55151293 | 55151362 | 431_48546_1956(EGFR)_6 | 0 | — |
| chr7 | 55152545 | 55152664 | 431_48547_1956(EGFR)_7 | 0 | — |
| chr7 | 55154010 | 55154152 | 431_48548_1956(EGFR)_8 | 0 | — |
| chr7 | 55155829 | 55155946 | 431_48549_1956(EGFR)_9 | 0 | — |
| chr7 | 55156532 | 55156659 | 431_48550_1956(EGFR)_10 | 0 | — |
| chr7 | 55156758 | 55156951 | 431_48551_1956(EGFR)_11 | 0 | — |
| chr7 | 55160138 | 55160338 | 431_48553_1956(EGFR)_13 | 0 | — |
| chr7 | 55161498 | 55161631 | 431_48554_1956(EGFR)_14 | 0 | — |
| chr7 | 55163732 | 55163823 | 431_48555_1956(EGFR)_15 | 0 | — |
| chr1 | 162741585 | 162741712 | 431_48985_4921(DDR2)_7a | 0 | — |
| chr1 | 162767228 | 162767359 | 431_48993_4921(DDR2)_15a | 0 | — |
| chr1 | 162780111 | 162780246 | 431_49000_4921(DDR2)_22a | 0 | — |
| chr1 | 162711291 | 162711300 | 431_48982_4921(DDR2)_4a | 0 | — |
| chr1 | 162719036 | 162719145 | 431_48983_4921(DDR2)_5a | 0 | — |
| chr1 | 162719190 | 162719214 | 431_48984_4921(DDR2)_6a | 0 | — |
| chr1 | 162753094 | 162753197 | 431_48986_4921(DDR2)_8a | 0 | — |
| chr1 | 162754623 | 162754855 | 431_48987_4921(DDR2)_9a | 0 | — |
| chr1 | 162755155 | 162755303 | 431_48988_4921(DDR2)_10a | 0 | — |
| chr1 | 162755663 | 162755769 | 431_48989_4921(DDR2)_11a | 0 | — |
| chr1 | 162759795 | 162759979 | 431_48990_4921(DDR2)_12a | 0 | — |
| chr1 | 162761210 | 162761454 | 431_48991_4921(DDR2)_13a | 0 | — |
| chr1 | 162766000 | 162766063 | 431_48992_4921(DDR2)_14a | 0 | — |
| chr1 | 162770301 | 162770512 | 431_48994_4921(DDR2)_16a | 0 | — |
| chr1 | 162772023 | 162772247 | 431_48995_4921(DDR2)_17a | 0 | — |
| chr1 | 162773468 | 162773596 | 431_48996_4921(DDR2)_18a | 0 | — |
| chr1 | 162775651 | 162775843 | 431_48997_4921(DDR2)_19a | 0 | — |
| chr1 | 162776135 | 162776370 | 431_48998_4921(DDR2)_20a | 0 | — |
| chr1 | 162778579 | 162778729 | 431_48999_4921(DDR2)_21a | 0 | — |
| chr3 | 41225333 | 41225572 | 429_36859_1499(CTNNB1)_10 | 0 | — |
| chr3 | 41236587 | 41236709 | 429_36867_1499(CTNNB1)_18 | 0 | — |
| chr3 | 41224068 | 41224081 | 429_36856_1499(CTNNB1)_7 | 0 | — |
| chr3 | 41224525 | 41224753 | 429_36857_1499(CTNNB1)_8 | 0 | — |
| chr3 | 41224953 | 41225207 | 429_36858_1499(CTNNB1)_9 | 0 | — |
| chr12 | 25245273 | 25245395 | 464_89186_3845(KRAS)_2 | 0 | — |
| chr12 | 25227233 | 25227412 | 464_89187_3845(KRAS)_3 | 0 | — |
| chr12 | 25225613 | 25225773 | 464_89188_3845(KRAS)_4 | 0 | — |
| chr3 | 41225659 | 41225861 | 429_36860_1499(CTNNB1)_11 | 0 | — |
| chr3 | 41227207 | 41227352 | 429_36861_1499(CTNNB1)_12 | 0 | — |
| chr3 | 41233340 | 41233444 | 429_36862_1499(CTNNB1)_13 | 0 | — |
| chr3 | 41233528 | 41233867 | 429_36863_1499(CTNNB1)_14 | 0 | — |
| chr3 | 41234138 | 41234297 | 429_36864_1499(CTNNB1)_15 | 0 | — |
| chr12 | 25215149 | 25215560 | 464_89189_3845(KRAS)_5 | 0 | — |
| chr12 | 25209510 | 25209911 | 464_89190_3845(KRAS)_6 | 0 | — |
| chr3 | 41235723 | 41235843 | 429_36865_1499(CTNNB1)_16 | 0 | — |
| chr3 | 41236348 | 41236499 | 429_36866_1499(CTNNB1)_17 | 0 | — |
| chr3 | 41238015 | 41238076 | 429_36868_1499(CTNNB1)_19 | 0 | — |
| chr3 | 41239133 | 41239342 | 429_36869_1499(CTNNB1)_20 | 0 | — |
| chr5 | 150073300 | 150073493 | 429_36458_1436(CSF1R)_7a | 0 | — |
| chr5 | 150059699 | 150059862 | 429_36466_1436(CSF1R)_15a | 0 | — |
| chr9 | 21971328 | 21971400 | 428_25765_1029(CDKN2A)_4a | 0 | — |
| chr9 | 21994138 | 21994454 | 428_25762_1029(CDKN2A)_1a | 0 | — |
| chr9 | 21991594 | 21992480 | 428_25763_1029(CDKN2A)_2a | 0 | — |
| chr9 | 21974403 | 21975133 | 428_25764_1029(CDKN2A)_3a | 0 | — |
| chr9 | 21970901 | 21971208 | 428_25766_1029(CDKN2A)_5a | 0 | — |
| chr9 | 21968469 | 21968771 | 428_25767_1029(CDKN2A)_6a | 0 | — |
| chr9 | 21968228 | 21968242 | 428_25768_1029(CDKN2A)_7a | 0 | — |
| chr12 | 12717839 | 12718314 | 428_25760_1027(CDKN1B)_1a | 0 | — |
| chr12 | 12718824 | 12718946 | 428_25761_1027(CDKN1B)_2a | 0 | — |
| chr12 | 112419111 | 112419125 | 472_140049_5781(PTPN11)_1a | 0 | — |
| chr12 | 112450317 | 112450512 | 472_140051_5781(PTPN11)_3a | 0 | — |
| chr12 | 112453194 | 112453387 | 472_140052_5781(PTPN11)_4a | 0 | — |
| chr12 | 112454563 | 112454680 | 472_140053_5781(PTPN11)_5a | 0 | — |
| chr12 | 112455949 | 112456063 | 472_140054_5781(PTPN11)_6a | 0 | — |
| chr12 | 112472943 | 112473040 | 472_140055_5781(PTPN11)_7a | 0 | — |
| chr12 | 112477650 | 112477730 | 472_140056_5781(PTPN11)_8a | 0 | — |
| chr12 | 112477856 | 112478015 | 472_140057_5781(PTPN11)_9a | 0 | — |
| chr12 | 112486474 | 112486923 | 472_140059_5781(PTPN11)_11a | 0 | — |
| chr12 | 112488442 | 112488510 | 472_140060_5781(PTPN11)_12a | 0 | — |
| chr12 | 112489023 | 112489175 | 472_140061_5781(PTPN11)_13a | 0 | — |
| chr12 | 112502143 | 112502256 | 472_140062_5781(PTPN11)_14a | 0 | — |
| chr12 | 112504694 | 112504764 | 472_140063_5781(PTPN11)_15a | 0 | — |
| chr10 | 87864468 | 87864547 | 472_139786_5728(PTEN)_1 | 0 | — |
| chr10 | 87894024 | 87894109 | 472_139787_5728(PTEN)_2 | 0 | — |
| chr10 | 87925512 | 87925557 | 472_139788_5728(PTEN)_3 | 0 | — |
| chr10 | 87931045 | 87931089 | 472_139789_5728(PTEN)_4 | 0 | — |
| chr10 | 87933012 | 87933251 | 472_139790_5728(PTEN)_5 | 0 | — |
| chr10 | 87957852 | 87958019 | 472_139792_5728(PTEN)_7 | 0 | — |
| chr10 | 87960893 | 87961118 | 472_139793_5728(PTEN)_8 | 0 | — |
| chr10 | 87965286 | 87965472 | 472_139794_5728(PTEN)_9 | 0 | — |
| chr1 | 11259247 | 11259415 | 466_105881_2475(MTOR)_20 | 0 | — |
| chr1 | 11256932 | 11257165 | 466_105883_2475(MTOR)_4a | 0 | — |
| chr1 | 11256284 | 11256302 | 466_105884_2475(MTOR)_5a | 0 | — |
| chr1 | 11255991 | 11256192 | 466_105885_2475(MTOR)_6a | 0 | — |
| chr1 | 11253838 | 11253973 | 466_105886_2475(MTOR)_7a | 0 | — |
| chr1 | 11247818 | 11248094 | 466_105887_2475(MTOR)_8a | 0 | — |
| chr1 | 11247624 | 11247733 | 466_105888_2475(MTOR)_9a | 0 | — |
| chr1 | 11241552 | 11241681 | 466_105890_2475(MTOR)_11a | 0 | — |
| chr1 | 11240302 | 11240547 | 466_105891_2475(MTOR)_12a | 0 | — |
| chr1 | 11238401 | 11238617 | 466_105892_2475(MTOR)_13a | 0 | — |
| chr7 | 152224014 | 152224179 | 464_94983_58508(KMT2C)_20 | 0 | — |
| chr7 | 152222000 | 152222066 | 464_94985_58508(KMT2C)_22 | 0 | — |
| chr1 | 11237842 | 11238048 | 466_105893_2475(MTOR)_14a | 0 | — |
| chr1 | 11234142 | 11234265 | 466_105894_2475(MTOR)_15a | 0 | — |
| chr7 | 152220522 | 152220735 | 464_94986_58508(KMT2C)_23 | 0 | — |
| chr1 | 11233397 | 11233487 | 466_105895_2475(MTOR)_16a | 0 | — |
| chr1 | 11232435 | 11232528 | 466_105896_2475(MTOR)_17a | 0 | — |
| chr1 | 11230924 | 11231054 | 466_105898_2475(MTOR)_19a | 0 | — |
| chr1 | 11228667 | 11228918 | 466_105899_2475(MTOR)_20a | 0 | — |
| chr1 | 11216147 | 11216234 | 466_105900_2475(MTOR)_21a | 0 | — |
| chr1 | 11213398 | 11213566 | 466_105901_2475(MTOR)_22a | 0 | — |
| chr1 | 11212795 | 11212908 | 466_105902_2475(MTOR)_23a | 0 | — |
| chr1 | 11212311 | 11212474 | 466_105903_2475(MTOR)_24a | 0 | — |
| chr7 | 152207299 | 152207428 | 464_94987_58508(KMT2C)_24 | 0 | — |
| chr1 | 11210813 | 11210906 | 466_105904_2475(MTOR)_25a | 0 | — |
| chr1 | 11204560 | 11204703 | 466_105906_2475(MTOR)_27a | 0 | — |
| chr1 | 11199540 | 11199703 | 466_105907_2475(MTOR)_28a | 0 | — |
| chr1 | 11199257 | 11199403 | 466_105908_2475(MTOR)_29a | 0 | — |
| chr7 | 152205105 | 152205225 | 464_94988_58508(KMT2C)_25 | 0 | — |
| chr7 | 152202933 | 152203064 | 464_94989_58508(KMT2C)_26 | 0 | — |
| chr7 | 152199278 | 152199459 | 464_94990_58508(KMT2C)_27 | 0 | — |
| chr7 | 152195906 | 152196011 | 464_94991_58508(KMT2C)_28 | 0 | — |
| chr7 | 152195521 | 152195569 | 464_94992_58508(KMT2C)_29 | 0 | — |
| chr7 | 152194228 | 152194261 | 464_94994_58508(KMT2C)_31 | 0 | — |
| chr7 | 152194008 | 152194128 | 464_94995_58508(KMT2C)_32 | 0 | — |
| chr7 | 152187714 | 152187847 | 464_94996_58508(KMT2C)_33 | 0 | — |
| chr7 | 152187261 | 152187476 | 464_94997_58508(KMT2C)_34 | 0 | — |
| chr7 | 152185557 | 152185631 | 464_94998_58508(KMT2C)_35 | 0 | — |
| chr7 | 152182973 | 152183156 | 464_94999_58508(KMT2C)_36 | 0 | — |
| chr1 | 11167441 | 11167517 | 466_105909_2475(MTOR)_30a | 0 | — |
| chr1 | 11157151 | 11157291 | 466_105910_2475(MTOR)_31a | 0 | — |
| chr1 | 11150125 | 11150226 | 466_105911_2475(MTOR)_32a | 0 | — |
| chr1 | 11144967 | 11145045 | 466_105913_2475(MTOR)_34a | 0 | — |
| chr1 | 11144647 | 11144755 | 466_105914_2475(MTOR)_35a | 0 | — |
| chr1 | 11139528 | 11139658 | 466_105915_2475(MTOR)_36a | 0 | — |
| chr1 | 11139303 | 11139435 | 466_105916_2475(MTOR)_37a | 0 | — |
| chr1 | 11134350 | 11134466 | 466_105917_2475(MTOR)_38a | 0 | — |
| chr1 | 11133079 | 11133197 | 466_105918_2475(MTOR)_39a | 0 | — |
| chr1 | 11130528 | 11130777 | 466_105919_2475(MTOR)_40a | 0 | — |
| chr1 | 11128854 | 11128951 | 466_105921_2475(MTOR)_42a | 0 | — |
| chr1 | 11128453 | 11128552 | 466_105922_2475(MTOR)_43a | 0 | — |
| chr1 | 11128003 | 11128126 | 466_105923_2475(MTOR)_44a | 0 | — |
| chr1 | 11127623 | 11127806 | 466_105924_2475(MTOR)_45a | 0 | — |
| chr1 | 11127009 | 11127144 | 466_105925_2475(MTOR)_46a | 0 | — |
| chr1 | 11126621 | 11126796 | 466_105926_2475(MTOR)_47a | 0 | — |
| chr1 | 11124497 | 11124633 | 466_105927_2475(MTOR)_48a | 0 | — |
| chr1 | 11121245 | 11121368 | 466_105929_2475(MTOR)_50a | 0 | — |
| chr1 | 11117003 | 11117086 | 466_105930_2475(MTOR)_51a | 0 | — |
| chr5 | 56820391 | 56820816 | 465_99619_4214(MAP3K1)_3a | 0 | — |
| chr5 | 56872824 | 56873005 | 465_99627_4214(MAP3K1)_11a | 0 | — |
| chr5 | 56885931 | 56886063 | 465_99635_4214(MAP3K1)_19a | 0 | — |
| chr5 | 56815573 | 56816055 | 465_99617_4214(MAP3K1)_1a | 0 | — |
| chr5 | 56817055 | 56817116 | 465_99618_4214(MAP3K1)_2a | 0 | — |
| chr5 | 56856599 | 56856750 | 465_99620_4214(MAP3K1)_4a | 0 | — |
| chr5 | 56859714 | 56859915 | 465_99621_4214(MAP3K1)_5a | 0 | — |
| chr5 | 56864733 | 56864934 | 465_99622_4214(MAP3K1)_6a | 0 | — |
| chr5 | 56865339 | 56865456 | 465_99623_4214(MAP3K1)_7a | 0 | — |
| chr5 | 56865828 | 56865977 | 465_99624_4214(MAP3K1)_8a | 0 | — |
| chr5 | 56871909 | 56872031 | 465_99625_4214(MAP3K1)_9a | 0 | — |
| chr5 | 56872640 | 56872722 | 465_99626_4214(MAP3K1)_10a | 0 | — |
| chr5 | 56875031 | 56875310 | 465_99628_4214(MAP3K1)_12a | 0 | — |
| chr5 | 56878979 | 56879101 | 465_99629_4214(MAP3K1)_13a | 0 | — |
| chr5 | 56880710 | 56880802 | 465_99630_4214(MAP3K1)_14a | 0 | — |
| chr5 | 56881082 | 56881272 | 465_99631_4214(MAP3K1)_15a | 0 | — |
| chr5 | 56881569 | 56882866 | 465_99632_4214(MAP3K1)_16a | 0 | — |
| chr5 | 56883526 | 56883679 | 465_99633_4214(MAP3K1)_17a | 0 | — |
| chr17 | 12020886 | 12021001 | 465_99947_6416(MAP2K4)_1 | 0 | — |
| chr17 | 12032265 | 12032298 | 465_99948_6416(MAP2K4)_2 | 0 | — |
| chr17 | 12054888 | 12054991 | 465_99949_6416(MAP2K4)_3 | 0 | — |
| chr12 | 120978768 | 120979094 | 463_81838_6927(HNF1A)_1 | 0 | — |
| chr12 | 120999267 | 120999627 | 463_81845_6927(HNF1A)_8 | 0 | — |
| chr17 | 12069723 | 12069849 | 465_99950_6416(MAP2K4)_4 | 0 | — |
| chr17 | 12081355 | 12081530 | 465_99951_6416(MAP2K4)_5 | 0 | — |
| chr17 | 12095574 | 12095694 | 465_99952_6416(MAP2K4)_6 | 0 | — |
| chr17 | 12107789 | 12107909 | 465_99953_6416(MAP2K4)_7 | 0 | — |
| chr17 | 12113232 | 12113360 | 465_99955_6416(MAP2K4)_9 | 0 | — |
| chr17 | 12125293 | 12125371 | 465_99956_6416(MAP2K4)_10 | 0 | — |
| chr17 | 12129138 | 12129287 | 465_99957_6416(MAP2K4)_11 | 0 | — |
| chr17 | 12139838 | 12139884 | 465_99958_6416(MAP2K4)_12 | 0 | — |
| chr17 | 12141146 | 12141260 | 465_99959_6416(MAP2K4)_13 | 0 | — |
| chr20 | 56382984 | 56383176 | 427_16076_6790(AURKA)_7a | 0 | — |
| chr20 | 56388155 | 56388197 | 427_16073_6790(AURKA)_4a | 0 | — |
| chr20 | 56386256 | 56386533 | 427_16074_6790(AURKA)_5a | 0 | — |
| chr20 | 56384269 | 56384324 | 427_16075_6790(AURKA)_6a | 0 | — |
| chr20 | 56381432 | 56381571 | 427_16077_6790(AURKA)_8a | 0 | — |
| chr20 | 56373407 | 56373556 | 427_16078_6790(AURKA)_9a | 0 | — |
| chr20 | 56370484 | 56370659 | 427_16079_6790(AURKA)_10a | 0 | — |
| chr20 | 56370157 | 56370340 | 427_16080_6790(AURKA)_11a | 0 | — |
| chr3 | 142578645 | 142578704 | 427_14853_545(ATR)_1 | 0 | — |
| chr3 | 142568062 | 142568154 | 427_14854_545(ATR)_2 | 0 | — |
| chr3 | 142566120 | 142566261 | 427_14855_545(ATR)_3 | 0 | — |
| chr3 | 142562231 | 142563109 | 427_14856_545(ATR)_4 | 0 | — |
| chr3 | 142561242 | 142561421 | 427_14857_545(ATR)_5 | 0 | — |
| chr3 | 142560262 | 142560454 | 427_14858_545(ATR)_6 | 0 | — |
| chr3 | 142558623 | 142558776 | 427_14860_545(ATR)_8 | 0 | — |
| chr3 | 142556382 | 142556575 | 427_14861_545(ATR)_9 | 0 | — |
| chr20 | 58852980 | 58855333 | 462_70271_2778(GNAS)_3 | 0 | — |
| chr20 | 58905382 | 58905480 | 462_70279_2778(GNAS)_11 | 0 | — |
| chr20 | 58840106 | 58840886 | 462_70269_2778(GNAS)_1 | 0 | — |
| chr3 | 142555876 | 142556139 | 427_14862_545(ATR)_10 | 0 | — |
| chr3 | 142553824 | 142554015 | 427_14863_545(ATR)_11 | 0 | — |
| chr3 | 142553639 | 142553740 | 427_14864_545(ATR)_12 | 0 | — |
| chr3 | 142553226 | 142553398 | 427_14865_545(ATR)_13 | 0 | — |
| chr3 | 142549478 | 142549673 | 427_14867_545(ATR)_15 | 0 | — |
| chr3 | 142547724 | 142547910 | 427_14868_545(ATR)_16 | 0 | — |
| chr3 | 142542664 | 142542757 | 427_14869_545(ATR)_17 | 0 | — |
| chr3 | 142540903 | 142541034 | 427_14870_545(ATR)_18 | 0 | — |
| chr3 | 142536107 | 142536201 | 427_14872_545(ATR)_20 | 0 | — |
| chr3 | 142535079 | 142535205 | 427_14873_545(ATR)_21 | 0 | — |
| chr3 | 142523992 | 142524199 | 427_14874_545(ATR)_22 | 0 | — |
| chr3 | 142522727 | 142522841 | 427_14875_545(ATR)_23 | 0 | — |
| chr3 | 142519668 | 142519784 | 427_14876_545(ATR)_24 | 0 | — |
| chr3 | 142515394 | 142515515 | 427_14877_545(ATR)_25 | 0 | — |
| chr3 | 142512259 | 142512470 | 427_14879_545(ATR)_27 | 0 | — |
| chr3 | 142507930 | 142508109 | 427_14880_545(ATR)_28 | 0 | — |
| chr3 | 142505138 | 142505303 | 427_14881_545(ATR)_29 | 0 | — |
| chr3 | 142503361 | 142503453 | 427_14882_545(ATR)_30 | 0 | — |
| chr3 | 142499626 | 142499718 | 427_14883_545(ATR)_31 | 0 | — |
| chr3 | 142498596 | 142498774 | 427_14884_545(ATR)_32 | 0 | — |
| chr20 | 58841342 | 58841875 | 462_70270_2778(GNAS)_2 | 0 | — |
| chr20 | 58889124 | 58889353 | 462_70272_2778(GNAS)_4 | 0 | — |
| chr20 | 58891370 | 58891865 | 462_70273_2778(GNAS)_5 | 0 | — |
| chr20 | 58892109 | 58892209 | 462_70274_2778(GNAS)_6 | 0 | — |
| chr20 | 58895611 | 58895684 | 462_70275_2778(GNAS)_7 | 0 | — |
| chr20 | 58898940 | 58898985 | 462_70276_2778(GNAS)_8 | 0 | — |
| chr20 | 58903527 | 58903585 | 462_70277_2778(GNAS)_9 | 0 | — |
| chr20 | 58903671 | 58903791 | 462_70278_2778(GNAS)_10 | 0 | — |
| chr3 | 142497012 | 142497192 | 427_14885_545(ATR)_33 | 0 | — |
| chr3 | 142493131 | 142493311 | 427_14887_545(ATR)_35 | 0 | — |
| chr3 | 142485139 | 142485282 | 427_14888_545(ATR)_36 | 0 | — |
| chr3 | 142470085 | 142470183 | 427_14889_545(ATR)_37 | 0 | — |
| chr3 | 142469336 | 142469569 | 427_14890_545(ATR)_38 | 0 | — |
| chr3 | 142467933 | 142468068 | 427_14891_545(ATR)_39 | 0 | — |
| chr3 | 142466323 | 142466533 | 427_14892_545(ATR)_40 | 0 | — |
| chr3 | 142465096 | 142465240 | 427_14893_545(ATR)_41 | 0 | — |
| chr3 | 142459226 | 142459383 | 427_14895_545(ATR)_43 | 0 | — |
| chr3 | 142458957 | 142459111 | 427_14896_545(ATR)_44 | 0 | — |
| chr3 | 142457603 | 142457755 | 427_14897_545(ATR)_45 | 0 | — |
| chr3 | 142450465 | 142453233 | 427_14898_545(ATR)_46 | 0 | — |
| chr20 | 58909161 | 58909216 | 462_70280_2778(GNAS)_12 | 0 | — |
| chr20 | 58909349 | 58909423 | 462_70281_2778(GNAS)_13 | 0 | — |
| chr20 | 58909520 | 58909579 | 462_70282_2778(GNAS)_14 | 0 | — |
| chr20 | 58909683 | 58909804 | 462_70283_2778(GNAS)_15 | 0 | — |
| chr20 | 58909950 | 58910081 | 462_70284_2778(GNAS)_16 | 0 | — |
| chr20 | 58910333 | 58910401 | 462_70285_2778(GNAS)_17 | 0 | — |
| chr20 | 58910682 | 58910829 | 462_70286_2778(GNAS)_18 | 0 | — |
| chr3 | 142449428 | 142449602 | 427_14899_545(ATR)_47 | 0 | — |
| chr9 | 78031099 | 78031235 | 462_70175_2776(GNAQ)_1 | 0 | — |
| chr11 | 108251836 | 108252031 | 427_14221_472(ATM)_15 | 0 | — |
| chr9 | 77815615 | 77815770 | 462_70177_2776(GNAQ)_3 | 0 | — |
| chr9 | 77797519 | 77797648 | 462_70178_2776(GNAQ)_4 | 0 | — |
| chr9 | 77794462 | 77794592 | 462_70179_2776(GNAQ)_5 | 0 | — |
| chr9 | 77728513 | 77728667 | 462_70180_2776(GNAQ)_6 | 0 | — |
| chr9 | 77721322 | 77721513 | 462_70181_2776(GNAQ)_7 | 0 | — |
| chr19 | 3114943 | 3115072 | 462_70070_2767(GNA11)_4 | 0 | — |
| chr11 | 108268409 | 108268609 | 427_14228_472(ATM)_22 | 0 | — |
| chr11 | 108293312 | 108293477 | 427_14241_472(ATM)_35 | 0 | — |
| chr11 | 108310159 | 108310315 | 427_14249_472(ATM)_43 | 0 | — |
| chr11 | 108325309 | 108325544 | 427_14256_472(ATM)_50 | 0 | — |
| chr11 | 108333885 | 108333968 | 427_14264_472(ATM)_58 | 0 | — |
| chr11 | 108354810 | 108354874 | 427_14271_472(ATM)_65 | 0 | — |
| chr19 | 17841388 | 17841546 | 463_81750_3718(JAK3)_8a | 0 | — |
| chr19 | 17834851 | 17835003 | 463_81758_3718(JAK3)_16a | 0 | — |
| chr19 | 17826742 | 17826910 | 463_81766_3718(JAK3)_24a | 0 | — |
| chr2 | 212538448 | 212538530 | 461_58772_2066(ERBB4)_1a | 0 | — |
| chr2 | 211947429 | 211947616 | 461_58774_2066(ERBB4)_3a | 0 | — |
| chr2 | 211788024 | 211788159 | 461_58775_2066(ERBB4)_4a | 0 | — |
| chr2 | 211750638 | 211750704 | 461_58776_2066(ERBB4)_5a | 0 | — |
| chr2 | 211725075 | 211725194 | 461_58777_2066(ERBB4)_6a | 0 | — |
| chr2 | 211722392 | 211722534 | 461_58778_2066(ERBB4)_7a | 0 | — |
| chr2 | 211713534 | 211713648 | 461_58779_2066(ERBB4)_8a | 0 | — |
| chr2 | 211712049 | 211712176 | 461_58780_2066(ERBB4)_9a | 0 | — |
| chr2 | 211705317 | 211705391 | 461_58781_2066(ERBB4)_10a | 0 | — |
| chr2 | 211701966 | 211702166 | 461_58783_2066(ERBB4)_12a | 0 | — |
| chr2 | 211679051 | 211679184 | 461_58784_2066(ERBB4)_13a | 0 | — |
| chr2 | 211673163 | 211673257 | 461_58785_2066(ERBB4)_14a | 0 | — |
| chr2 | 211665322 | 211665477 | 461_58786_2066(ERBB4)_15a | 0 | — |
| chr2 | 211657949 | 211657994 | 461_58787_2066(ERBB4)_16a | 0 | — |
| chr12 | 56093763 | 56093896 | 461_58756_2065(ERBB3)_13a | 0 | — |
| chr12 | 56094401 | 56094556 | 461_58758_2065(ERBB3)_15a | 0 | — |
| chr12 | 56095256 | 56095310 | 461_58759_2065(ERBB3)_16a | 0 | — |
| chr12 | 56095664 | 56095806 | 461_58760_2065(ERBB3)_17a | 0 | — |
| chr12 | 56096502 | 56096622 | 461_58761_2065(ERBB3)_18a | 0 | — |
| chr12 | 56096747 | 56096846 | 461_58762_2065(ERBB3)_19a | 0 | — |
| chr12 | 56097044 | 56097230 | 461_58763_2065(ERBB3)_20a | 0 | — |
| chr12 | 56097784 | 56097940 | 461_58764_2065(ERBB3)_21a | 0 | — |
| chr12 | 56098758 | 56098905 | 461_58766_2065(ERBB3)_23a | 0 | — |
| chr7 | 55165279 | 55165437 | 431_48556_1956(EGFR)_16 | 0 | — |
| chr7 | 55170306 | 55171045 | 431_48558_1956(EGFR)_18 | 0 | — |
| chr7 | 55171174 | 55171213 | 431_48559_1956(EGFR)_19 | 0 | — |
| chr7 | 55172845 | 55172957 | 431_48560_1956(EGFR)_20 | 0 | — |
| chr7 | 55172982 | 55173124 | 431_48561_1956(EGFR)_21 | 0 | — |
| chr7 | 55173920 | 55174043 | 431_48562_1956(EGFR)_22 | 0 | — |
| chr7 | 55174721 | 55174820 | 431_48563_1956(EGFR)_23 | 0 | — |
| chr7 | 55181292 | 55181478 | 431_48564_1956(EGFR)_24 | 0 | — |
| chr7 | 55192765 | 55192841 | 431_48566_1956(EGFR)_26 | 0 | — |
| chr7 | 55198716 | 55198863 | 431_48567_1956(EGFR)_27 | 0 | — |
| chr7 | 55200315 | 55200413 | 431_48568_1956(EGFR)_28 | 0 | — |
| chr7 | 55201187 | 55201355 | 431_48569_1956(EGFR)_29 | 0 | — |
| chr7 | 55201734 | 55201782 | 431_48570_1956(EGFR)_30 | 0 | — |
| chr7 | 55202516 | 55202625 | 431_48571_1956(EGFR)_31 | 0 | — |
| chr7 | 55205255 | 55205617 | 431_48572_1956(EGFR)_32 | 0 | — |
| chr12 | 112446275 | 112446398 | 472_140050_5781(PTPN11)_2a | 0 | — |
| chr12 | 112482073 | 112482205 | 472_140058_5781(PTPN11)_10a | 0 | — |
| chr10 | 87952117 | 87952259 | 472_139791_5728(PTEN)_6 | 0 | — |
| chr1 | 11115395 | 11115468 | 466_105931_2475(MTOR)_52a | 0 | — |
| chr1 | 11114812 | 11114887 | 466_105932_2475(MTOR)_53a | 0 | — |
| chr1 | 11114317 | 11114453 | 466_105933_2475(MTOR)_54a | 0 | — |
| chr1 | 11112851 | 11112917 | 466_105934_2475(MTOR)_55a | 0 | — |
| chr1 | 11109289 | 11109370 | 466_105936_2475(MTOR)_57a | 0 | — |
| chr1 | 11108180 | 11108286 | 466_105937_2475(MTOR)_58a | 0 | — |
| chr1 | 11106604 | 11107500 | 466_105938_2475(MTOR)_59a | 0 | — |
| chr19 | 3094651 | 3094787 | 462_70067_2767(GNA11)_1 | 0 | — |
| chr19 | 3110148 | 3110333 | 462_70068_2767(GNA11)_2 | 0 | — |
| chr19 | 3113329 | 3113484 | 462_70069_2767(GNA11)_3 | 0 | — |
| chr19 | 3118923 | 3119053 | 462_70071_2767(GNA11)_5 | 0 | — |
| chr11 | 108227624 | 108227696 | 427_14211_472(ATM)_5 | 0 | — |
| chr11 | 108227775 | 108227888 | 427_14212_472(ATM)_6 | 0 | — |
| chr19 | 3119205 | 3119359 | 462_70072_2767(GNA11)_6 | 0 | — |
| chr19 | 3120988 | 3121179 | 462_70073_2767(GNA11)_7 | 0 | — |
| chr11 | 108229177 | 108229323 | 427_14213_472(ATM)_7 | 0 | — |
| chr11 | 108243952 | 108244118 | 427_14215_472(ATM)_9 | 0 | — |
| chr11 | 108244787 | 108245026 | 427_14216_472(ATM)_10 | 0 | — |
| chr11 | 108246543 | 108246560 | 427_14217_472(ATM)_11 | 0 | — |
| chr11 | 108246963 | 108247127 | 427_14218_472(ATM)_12 | 0 | — |
| chr11 | 108248932 | 108249102 | 427_14219_472(ATM)_13 | 0 | — |
| chr11 | 108250700 | 108251072 | 427_14220_472(ATM)_14 | 0 | — |
| chr11 | 108252816 | 108252912 | 427_14222_472(ATM)_16 | 0 | — |
| chr11 | 108253813 | 108254039 | 427_14223_472(ATM)_17 | 0 | — |
| chr11 | 108256214 | 108256340 | 427_14224_472(ATM)_18 | 0 | — |
| chr11 | 108257480 | 108257606 | 427_14225_472(ATM)_19 | 0 | — |
| chr11 | 108258985 | 108259075 | 427_14226_472(ATM)_20 | 0 | — |
| chr11 | 108267170 | 108267342 | 427_14227_472(ATM)_21 | 0 | — |
| chr11 | 108271063 | 108271146 | 427_14229_472(ATM)_23 | 0 | — |
| chr11 | 108271250 | 108271406 | 427_14230_472(ATM)_24 | 0 | — |
| chr11 | 108272531 | 108272607 | 427_14231_472(ATM)_25 | 0 | — |
| chr11 | 108272721 | 108272852 | 427_14232_472(ATM)_26 | 0 | — |
| chr11 | 108279490 | 108279608 | 427_14233_472(ATM)_27 | 0 | — |
| chr11 | 108280994 | 108281168 | 427_14234_472(ATM)_28 | 0 | — |
| chr11 | 108282709 | 108282879 | 427_14235_472(ATM)_29 | 0 | — |
| chr11 | 108284226 | 108284473 | 427_14236_472(ATM)_30 | 0 | — |
| chr11 | 108287599 | 108287715 | 427_14237_472(ATM)_31 | 0 | — |
| chr11 | 108288976 | 108289103 | 427_14238_472(ATM)_32 | 0 | — |
| chr11 | 108289601 | 108289801 | 427_14239_472(ATM)_33 | 0 | — |
| chr11 | 108292618 | 108292793 | 427_14240_472(ATM)_34 | 0 | — |
| chr11 | 108294926 | 108295059 | 427_14242_472(ATM)_36 | 0 | — |
| chr11 | 108297286 | 108297382 | 427_14243_472(ATM)_37 | 0 | — |
| chr11 | 108299713 | 108299885 | 427_14244_472(ATM)_38 | 0 | — |
| chr11 | 108301647 | 108301789 | 427_14245_472(ATM)_39 | 0 | — |
| chr11 | 108302852 | 108303029 | 427_14246_472(ATM)_40 | 0 | — |
| chr11 | 108304674 | 108304852 | 427_14247_472(ATM)_41 | 0 | — |
| chr11 | 108307896 | 108307984 | 427_14248_472(ATM)_42 | 0 | — |
| chr11 | 108312410 | 108312498 | 427_14250_472(ATM)_44 | 0 | — |
| chr11 | 108315822 | 108315911 | 427_14251_472(ATM)_45 | 0 | — |
| chr11 | 108316010 | 108316113 | 427_14252_472(ATM)_46 | 0 | — |
| chr11 | 108317372 | 108317521 | 427_14253_472(ATM)_47 | 0 | — |
| chr11 | 108319953 | 108320058 | 427_14254_472(ATM)_48 | 0 | — |
| chr11 | 108321300 | 108321420 | 427_14255_472(ATM)_49 | 0 | — |
| chr11 | 108326057 | 108326225 | 427_14257_472(ATM)_51 | 0 | — |
| chr11 | 108327644 | 108327758 | 427_14258_472(ATM)_52 | 0 | — |
| chr11 | 108329020 | 108329238 | 427_14259_472(ATM)_53 | 0 | — |
| chr11 | 108330213 | 108330421 | 427_14260_472(ATM)_54 | 0 | — |
| chr11 | 108331443 | 108331557 | 427_14261_472(ATM)_55 | 0 | — |
| chr11 | 108331878 | 108332037 | 427_14262_472(ATM)_56 | 0 | — |
| chr11 | 108332761 | 108332900 | 427_14263_472(ATM)_57 | 0 | — |
| chr11 | 108334968 | 108335109 | 427_14265_472(ATM)_59 | 0 | — |
| chr11 | 108335844 | 108335961 | 427_14266_472(ATM)_60 | 0 | — |
| chr11 | 108343221 | 108343371 | 427_14267_472(ATM)_61 | 0 | — |
| chr11 | 108345742 | 108345908 | 427_14268_472(ATM)_62 | 0 | — |
| chr11 | 108347278 | 108347365 | 427_14269_472(ATM)_63 | 0 | — |
| chr11 | 108353765 | 108353880 | 427_14270_472(ATM)_64 | 0 | — |
| chr7 | 55143304 | 55143488 | 431_48544_1956(EGFR)_4 | 0 | — |
| chr7 | 55157662 | 55157753 | 431_48552_1956(EGFR)_12 | 0 | — |
| chr7 | 55168522 | 55168635 | 431_48557_1956(EGFR)_17 | 0 | — |
| chr7 | 55191718 | 55191874 | 431_48565_1956(EGFR)_25 | 0 | — |
| chr5 | 150086378 | 150086427 | 429_36453_1436(CSF1R)_2a | 0 | — |
| chr5 | 150080766 | 150081024 | 429_36454_1436(CSF1R)_3a | 0 | — |
| chr5 | 150080051 | 150080336 | 429_36455_1436(CSF1R)_4a | 0 | — |
| chr5 | 150078111 | 150078248 | 429_36456_1436(CSF1R)_5a | 0 | — |
| chr5 | 150077275 | 150077435 | 429_36457_1436(CSF1R)_6a | 0 | — |
| chr5 | 150070455 | 150070571 | 429_36459_1436(CSF1R)_8a | 0 | — |
| chr5 | 150070181 | 150070302 | 429_36460_1436(CSF1R)_9a | 0 | — |
| chr5 | 150069872 | 150070063 | 429_36461_1436(CSF1R)_10a | 0 | — |
| chr5 | 150068214 | 150068330 | 429_36462_1436(CSF1R)_11a | 0 | — |
| chr5 | 150061722 | 150061849 | 429_36463_1436(CSF1R)_12a | 0 | — |
| chr5 | 150061490 | 150061595 | 429_36464_1436(CSF1R)_13a | 0 | — |
| chr5 | 150060861 | 150060972 | 429_36465_1436(CSF1R)_14a | 0 | — |
| chr5 | 150057503 | 150057592 | 429_36467_1436(CSF1R)_16a | 0 | — |
| chr5 | 150057286 | 150057384 | 429_36468_1436(CSF1R)_17a | 0 | — |
| chr5 | 150056218 | 150056341 | 429_36469_1436(CSF1R)_18a | 0 | — |
| chr5 | 150056025 | 150056137 | 429_36470_1436(CSF1R)_19a | 0 | — |
| chr5 | 150055236 | 150055336 | 429_36471_1436(CSF1R)_20a | 0 | — |
| chr5 | 150054321 | 150054430 | 429_36472_1436(CSF1R)_21a | 0 | — |
| chr5 | 150054068 | 150054224 | 429_36473_1436(CSF1R)_22a | 0 | — |

### Supplementary Data 3

| SEQ | | |
|---|---|---|
| ID | Sequence | |
| NO | Name | Sequence |
| 1 | 632327_266823 | TGGCATTAGGGACCCCAAATCCAGGTCTGCTCCCAGTGCCATAGGAA |
| | 54_Target2.1_1 | GCAACCAGCCCCCAACCCCCACCTGCTCTTGGGAGCTGGCCCACAC |
| | | CAGAAATGGACTGAGTAGACATGAGAA |
| 2 | 632327_266823 | CTGACCCCAGCTTCCACCAATGCCGTTAAGATGCCGCCACTTGGGTG |
| | 54_Target2.1_2 | AGGGGCTCCTCCAGGTACTGCACCAAAGCCTGGGCCTGCAGAGGGA |
| | | GAACAGAGATCAGTCCCGCACACAGCC |
| 3 | 632327_266823 | CCAAGACACATGACAGGAGTTGAAGTTTATTCTTGGAAAAAACAAAGT |
| | 54_Target2.1_3 | CCCATCCTCCCCCCATTGTCTAAGAAGGTTCTTCTAGGAGGCCCCGC |
| | | CCCTCCAAATGGTCATTTCTCTTTT |
| 4 | 632327_266823 | CTACTTTCTTGGATGGGAGGAATCAGAGAAGATAGGAAGAAGCAGGC |
| | 54_Target2.1_4 | ATCGCTTCTAGGATTCCTCATGTTCTGCAAGGATGGACTTCTACCCAC |
| | | CCCTTCACCATGACCTTCTGCGAAC |
| 5 | 632327_266823 | AATGGTTCTTGGCAGTTACGCCAAATAGTCCTTGTCCTCACCAATGG |
| | 54_Target2.1_5 | CATGACCTCACCTAGACCCTGCTGCTCAGAACAGAGTCTTACCCACC |
| | | TCATAGGCAAAACTGGGAGTGAGCTC |
| 6 | 632327_266823 | AGACCCTCTCAAAAAAATAAAAGGCACGCACGCCCCCAAACCTGAAA |
| | 54_Target2.1_6 | TGATAGACACTAACCTCTCAGGGCCTTTATATTCTCTGTGCAACACAA |
| | | CCCCCACCAGACCCTTGCACCTCT |
| 7 | 632327_266823 | TGTGTAAGCACATAAACACCTGGAAGAAGAAGAAAGGGATAGCTTGT |
| | 55_Target2.2_1 | TCTAGGCTGTTAGGGGCACCCTCCCAACCCCAAGAATAGAAAAGGCA |
| | | CTCACTGGCCAGTCGAAGTGGTTCAT |
| 8 | 632327_266823 | GTGTTTATGTGCTTACACAGACCCTCCCCAGATCTTGAAGCCCTTTGT |
| | 55_Target2.2_2 | AGACAAGAGGCCTTTGCTGTGAGGCCTCAGGTCCTGTACAGGGAGT |
| | | AGAAGTAACCCAGACCTCCCAAAGGC |
| 9 | 632327_266823 | ACCCAGACCTCCCAAAGGCCTTCATCTCCAGACCCTGCTCCTACACT |
| | 55_Target2.2_3 | CAGACCACTCACCCCAGCTGTCTGTTTCTGCCCCAGGTCTCTGCCTA |
| | | CCCAATATCCTTAAAGCAGGTCTGTA |
| 10 | 632327_266823 | TCCTTAAAGCAGGTCTGTATAGGGAGCTACCAGCCCCCTTCCCCAGT |
| | 55_Target2.2_4 | GCCATATAAAGGAACCCATCCCAGAGAAGTGGGTAGGTTTCGAGTCC |
| | | CAGCTCCACCCAAGATGAGAAACACT |
| 11 | 632327_266823 | ACCCAAGATGAGAAACACTGCCCATCCCAGGCCCTGGGCTGTCAAG |
| | 55_Target2.2_5 | GAAGGGCAGAAGTTACTGTGGCCATGATACTCCCACAGCTCTGCATT |
| | | GCAGGAGGGTGACTGGTGTTCTATCAG |
| 12 | 632327_266823 | GTGACTGGTGTTCTATCAGCCCCTGGTCTAATATCCCTTACTCTGGTT |
| | 55_Target2.2_6 | CCCCCAGTGTACCCCCCTACCAGCTCTGAAGGAAAAGCAGGCCAAG |
| | | TGGGTTTCTTCCTTTTTTTTTTTTTT |
| 13 | 632327_266823 | CACTGAGGGCTGAGTGTGCATTGCTGGGCTTCTGGAATCTGTGGTGA |
| | 56_Target2.3_1 | GGGCCGGTCCCCCAACTAATGAGCAAGAAATAGCCCCTCAGAAACC |
| | | CACTTGAGGCCAGGCGCGGTGGCTCAC |
| 14 | 632327_266823 | GTGGGCTGTCTCTGAAGGTGGTGTGGGTTTTTGGGTTGGGGGCGGGT |
| | 56_Target2.3_2 | GGAGCTGCTTCTCCCGCTTGCGGGAGCCCAGGCTGAGAGCAGACAC |
| | | CCAACCTGTCGAACCTGTCTGACGTCA |
| 15 | 632327_266823 | TCATCATCTCTCCACCCACCTGGGCCCCAGGTCTCCAGCCACCCCG |
| | 56_Target2.3_3 | CTCTTCCTGTTCTCAGCTTCCGTCCTCTCTGCTTCCTTACAGCACCCC |
| | | CACCTGCCAGAGCTGATCCTCCCTAG |
| 16 | 632327_266823 | GCTCAATGCCCTCAACCCTCAGCTCTCAAAAGATCTGGTCCTGCCCC |
| | 56_Target2.3_4 | TCCTCTCATTGGGGGTAAGGGGACTTCTGCAGCCAGAGGGATTGGG |
| | | GGCCAACTCAAGGTTAGGCAGGGCCTA |
| 17 | 632327_266823 | GAACCCAACTCACCCTTGAAGACAAAGTTATAATCTTCCTCAGTTCCA |
| | 56_Target2.3_5 | TTCCCCATCTTGGCTCCGCATGGAGGGTGCAGGTGTCTTCGGGGAC |
| | | AGAGGCGACAAATCTGTGTGTTGGCT |
| 18 | 632327_266823 | TTCTGAAGGAATGAGGAGCCCCAGGAGGTCTTGGTAAGAGATAAAAA |
| | 56_Target2.3_6 | AACAGGAGGGGGCCCTCTCTGGCTCTATTCAATCTCTTTCTGGGTCT |
| | | CTCAGGCTTCCTCTTGCTTCAGGGAA |
| 19 | 632327_266823 | TAACTCAGCACTCTTAAGGAGGAATCAACATCATTATTTACCACAGTA |
| | 56_Target2.3_7 | GAGCATACTCAGAAGCTCCCTGACTTTTGCCCCTGCAACCAGACCCT |
| | | CCCCCACTCCCTGCAGCTTTCTTTC |
| 20 | 632327_266823 | TTAGGCTCCTCTGCCCCGTTCCTAATAGAAACCCAAATCCCTGGTGT |
| | 56_Target2.3_8 | CAGCCAGTGCAGCACAAGAGATGGAGGTCAGACTTCAGAAAGGACT |
| | | TCCCTCTTTTATTTTTTTTTCTGAGA |
| 21 | 632327_266823 | ATGAGCCACCACACCCGGCCAGGACTTCTCTCTTGAGAAGAAAGGG |
| | 57_Target2.4_1 | AAACCAGCAAACAAGAGGAAGAAAGTCATCTTTTCTGGGCAGGTGTA |
| | | CAGGGAACGAGAGAAAGGAGAGACACA |
| 22 | 632327_266823 | CAGACGAGGCTACAACCCTAGGAGTCCTCTGGATTCTATCAGACTCT |
| | 57_Target2.4_2 | GCAGCAAATTCATGATATTTTCTGTCTTCCCTATGTATTTATTGCCCCA |
| | | CCCTATAATGGGATGTGTCTCTCC |
| 23 | 632327_266823 | AGCCTCGTCTGCCTGGCCCTTGGCTGACTCACTGGACCTGCATACCA |
| | 57_Target2.4_3 | GGCACTGGGCTCAACTTTGCTGTCCCTGGAAGCATGCATGTATCTTT |
| | | CTAGCCCCTGTGTTTGGTGCCTTTGC |
| 24 | 632327_266823 | GGTGCCTTTGCTGTTTTCCAACCCCACCTTCATACTTCCTTTAGGGCA |
| | 57_Target2.4_4 | GGGTGGACTCTCCAGCCACCCTACCCCAATGCCTCTCCCAACCTCAG |
| | | CCCGGCCCCTCCCTTGAGCTGGGAC |
| 25 | 632327_266823 | AATGGTAGTTCCTCATATTATTGCCCCAGACTATTGCTAACCCTTTCC |
| | 57_Target2.4_5 | TTCCTGGAAAAGATGCCAGTAGTACAGGTTCCCCAGCTCAAGGGCG |
| | | GGTTCTGGAGTGAGAGTCCCAGCTCA |
| 26 | 632327_266823 | TCCCCTACTGGGCACATCTCCCTCATCCCTCTCAGTGGGTGGGCTCT |
| | 58_Target2.5_1 | CAGCCCTAGCGTCATACCACAAAGGTGCTTAGGGCAAGGGAATTGAA |
| | | GTCAGACTTTGTGGCCTTTTGTAAAC |
| 27 | 632327_266823 | GTGTGTATATGTGTGTGTGTGTTCCTGCCTCTCCCCTACTGGGCACA |
| | 58_Target2.5_2 | TCTCCCTCATCCCTCTCAGTGGGTGGGCTCTCAGCCCTAGCGTCATA |
| | | CCACAAAGGTGCTTAGGGCAAGGGAA |
| 28 | 632327_266823 | CACACACGCATACACACACATCCTCTTACCCACACATATCCATATTAT |
| | 59_Target2.6_1 | TTCCAGCTTTTCCATACTGTCCTATGCAAATAAGGAATGTTTAAATATA |
| | | TATTTTTTATTTTTATTTTTTGA |
| 29 | 632327_266823 | CCAGGACCAAGTGGGGTGATCAGGACCCCTCCAGGTGGGTCCCGG |
| | 60_Target4.1_1 | GTGCGGATGGCCAGTTGGGCGCATGCTTATTGGGCCTGCATCCGTT |
| | | GGCCACGGAGGCTCTTTGGGACAAGCAAA |
| 30 | 632327_266823 | GGCCACCCTGTTGTTGTGGGAGTATTAGGGGAAGTTGCCACTAAGG |
| | 60_Target4.1_2 | CTGGCAGGTCCTGGAGTTCCACCCAGGATGGGGACATGGGGGAGG |
| | | GAAGAGGAGGTGCCCCTCAGAGCCTGAGG |
| 31 | 632327_266823 | GGATAGGAGCCATGCTCCCCTGGGGTGGCTCTGGAACGTGGGCCCC |
| | 60_Target4.1_3 | GTGCACAGGCTATGTAGTCTGCTCACACCCTTGCTGCCTGCCACCCG |
| | | GGCACCCAGGACTTGAGCCCAGACCCT |
| 32 | 632327_266823 | GCCACCCCACAGGTGGCTTTTTATTCCCTCCTGCTGCTGAGTGTCAC |
| | 60_Target4.1_4 | GCGTGGGGCTGAGGGCCTGCACGCCTTCCCGGCCTCATCCTCTCCG |
| | | ACGATTCACGCCCTCCTAAGGCTGGCA |
| 33 | 632327_266823 | CACTGCCAAGGGCTCCCCGACACAAGCACAGAGCACAGCCTGGCCT |
| | 60_Target4.1_5 | TGGCTGCAGCACCACAGCAGATGCCTTTATGCCCAGGGCCAGCTAT |
| | | GGGCTGAAAGCACACCCCAGTCCTCACT |
| 34 | 632327_266823 | TCCGCACTGTCTGTGGGGTGTTTTGGGTTGCAGGTTCCATGTCCTGA |
| | 60_Target4.1_6 | AGCAGGAGAGCCCTCCCCATGCCACCTCCTCTGCCCAGCCTCTTAG |
| | | GGACAGTGCCTGGGATGCCCTGGCTGG |
| 35 | 632327_266823 | CAGCCTCACACCGAGCTCTCTGCCAAAACCCCGGATCACAAGCATGA |
| | 60_Target4.1_7 | GCTCCAGCAGGTCCGCTTGAGTGAACACCGCTGATCCTGCCTGCAG |
| | | GGGTATAGGCCTGGGGCCTGCCAGCCG |
| 36 | 632327_266823 | TTCTAGCCTGGGGCAGAAGTAGGGTGGAGGGGTGGGTCTAGCCTCC |
| | 60_Target4.1_8 | TTGATGACACAGTTGGGGTTGGAGGTGGGGGCTACCCCTATTCTGG |
| | | GCCTGGTGCTGTCCCTGAAGTCAGGGGT |
| 37 | 632327_266823 | TGTAGAGGTGGGAGGAAGTCAGGGAAGCAAGACACAGGTGGAGCCA |
| | 60_Target4.1_9 | GGGCCTCTCCTGTGCTCGGAGGGCCAGGAAGGGCCTGACAGAGGG |
| | | CAGAGCCCCACCCTCTTACACGGAGACCA |
| 38 | 632327_266823 | AGGAGGCAGTTTTGGAGTCAGAGGTGAAGTGTGGGGAGTGGGATTG |
| | 60_Target4.1_10 | GGTGATAGGGAAGTTTCTCCAGCAAAGACACTGGGCAAGCCTCTTCC |
| | | TTGGCCCCCAGATAAAGGGCTGCAGGG |
| 39 | 632327_266823 | CCCTTTTCTTCTGAGCCTGGACAACTCCTTCCCTTGCCCCCAGGAGC |
| | 60_Target4.1_11 | CCACAGCCGGCTAAACCAACTGGTCAGCTAGAACCAACCAGCAGGG |
| | | GAGGTGCGCTCCAGGGGGGCTTCTAAG |
| 40 | 632327_266823 | TGGTAGGACCTCCTAAAGGGACAGCAGATGCCAGGAGAGGGCCGCA |
| | 60_Target4.1_12 | AAGGCACAGCGTTGGAACCAGAGGGTTCCTACCAAGTTGACAACTCC |
| | | AAAAGAGCATGGTGGTCCCCCAAGGAC |
| 41 | 632327_266823 | ACAGAGAGACAGTCAGTTGTCCTCAGAATTGGATCCAGAACCTCGTC |
| | 60_Target4.1_13 | TTCTGGCATGGCCCCCCAGTGCAGCACCACATGTGGCCTCCGAATTC |
| | | CCAGTCTGGGATTGGGGGAGCTCTG |
| 42 | 632327_266823 | TGGCCCCAGGTCTAGGCGAGGGATGGAAATAAGGACAGTGCCACTA |
| | 61_Target4.2_1 | GGGCCTCCTGTCTGTACAGGAGGGGCCCTGAGACAGCTCCTCAAAC |
| | | CCCTTTCCCATTATACGGATGGTGCAGA |
| 43 | 632327_266823 | GGCCATGGTACCAGTACACAGTGCTCACCCTCCTGCTCAGACAAACA |
| | 61_Target4.2_2 | GTAACGGCCAGAAGCTCCAAGCCTCCTGCCCCAGCCCAAGGTGACT |
| | | TTCCTGGCAGAACCTGGGACTGCCCTG |
| 44 | 632327_266823 | CCCTGCCCTGCCCTGCCTCCACCCAGCTCCCGGGCAAGCAGTGCCA |
| | 61_Target4.2_3 | GGAGGAAGTGTCAGCAGCTCCTCCCCAAAGCTCTTTTCCCCACAGCC |
| | | CCGCCACCCCCGTACCTGGGCCCCGAT |
| 45 | 632327_266823 | CCCATCGCCCTGGGCCGGGACACGCCCCCTCTTGTGCCCATTCCTT |
| | 61_Target4.2_4 | TCCCTCGGTGGAGAGTTGGGTGGTGGGGGCTGGCCGGCCTGTGAG |
| | | AGCAGGTGTCGGGGTGCCTGGAGAATCGG |
| 46 | 632327_266823 | TCCCTCCTGAGAGTCCCTGGTTTCCTTTCCCACACCCCTCCCGGGGC |
| | 61_Target4.2_5 | CCGCCGCAGCTTTGGTCACCTTCTCAGAATGACCATGGGTTGATCTC |
| | | TTCTTTGCTAGTGAGATGTTTCCCAT |
| 47 | 632327_266823 | AGGGAACGGGAGCCTTGTGGTAGAAAAGGAGCTGCTGTCCCCGATC |
| | 61_Target4.2_6 | TGGAACTCGAAACCCCAACATCTTCCAGGCCGTAAGCCCCTCACCCC |
| | | GCACCCCTAGGCAAATCTCCGCTTTCC |
| 48 | 632327_266823 | TTTCCCAACTCCCTCACCTCGGTCCAGCGATCAGCCTCTCGGACCCT |
| | 61_Target4.2_7 | CGAGAGGGAGCTCCGACCCTGCTCTAGCTCTTGCGGGTCCCCCAAC |
| | | CATGGACTCCAAAGCCTTCATGTCCAC |
| 49 | 632327_266823 | CGCCGCGGGCTTTGCAGCCCCGCACGCCGAGCGCCGGTGGAAGCC |
| | 61_Target4.2_8 | GGGTCCTGGCCACAGGAGTCCTAGCCGCACGCGAGTGTCGCGGGA |
| | | GGGGGCCAGGGCCGGGTTCTGCAGTGTGGA |
| 50 | 632327_266823 | CGGCGCGCCCGCCCCCACTCCCGCTCCGCTCCCGGAACCCCGCGC |
| | 61_Target4.2_9 | CGGCGGCCGGAGTGAATCATTAACTGGAGCCGGGTGCTCCGGCTGC |
| | | GCAGCCTTCCCCGCTGGCGCCACGCACTT |
| 51 | 632327_266823 | CACTTGCCCGCCCGGCCTCGGTCCCCCGGAGCTCCTCCCTCTCCGG |
| | 61_Target4.2_10 | GAGCCGGTGGGCAGTCCCCACGCCACACCAGCCGGACCACCCGCC |
| | | AGAGGCTCAACGGCCCCTCCTCGCAG |
| 52 | 632327_266823 | GCGTTGGCGGGGCCGGGCGCGGGGGCCGCCAGCCGGTAGGAGGG |
| | 62_Target4.3_1 | TTCCTGTGTCGGGAAAGTGGCTGGCGGGGCCCGCGCTCCCCGGCC |
| | | GACGGCCGCCTGCTGTGTCACCTTGGGCCTC |
| 53 | 632327_266823 | GGCCCCGCCAACGCACCCGAGGCGCACAGGCTCCGGAGCCCCCGC |
| | 62_Target4.3_2 | GGTTCCTGCCTCCCGGCTTCCCGGCCAGGGCAGAAGGAGATCCCAG |
| | | GGCCGCGGACCCCGGCCGGCGGTTCCCAG |
| 54 | 632327_266823 | CCGGCGGTTCCCAGGCCCCGCAGGTCCGGCAGGAAATGCCGCTCG |
| | 62_Target4.3_3 | CTTAACCCCTTCCAGCCCGCTGCCGCCTCCAAGCCCGTACGCGGCC |
| | | CGGCCCCACCTGGCGCCCTCACCAGGTGG |
| 55 | 632327_266823 | ACCGGGGCGGCTGCGGCGCGCTCGGAGCCCCGAGGGCACGCGGC |
| | 62_Target4.3_4 | CCGGGCAGCTCGGTGTGCGCCCCCGCGAGAGCCGGGGCCCCAGGC |
| | | CCGCCGGACACCATGAACCACCTGGTGAGGG |
| 56 | 632327_266823 | GCAGCCGCCCCGGTGCCGCCGCGCAGCTGCCGCCTCGGCCACCCA |
| | 62_Target4.3_5 | GAGCCGGTCCAGCCGCTCTCCGCCTGCCGCCACGGCCCAGCCGGC |
| | | CCAGCCCGATCCCAGCATGCCCGGCCGCGC |
| 57 | 632327_266823 | GGGGGCGGGGCGGGGGGCGGCCCCTACTGGGCGGGCTTCTGTCT |
| | 62_Target4.3_6 | GCGCGCCCGCGCCCCCGACAGGCGGCTTATTTGCATGGAGAGCGG |
| | | CGGCGGCCAATGGGCGCGCGCGGCCGGGCAT |
| 58 | 632327_266823 | GAGGCTCCAGGCCCCTGCCCGAGGCCAGGCTTCCAGCCTAGGGCG |
| | 63_Target4.4_1 | CAGGACACCGCTCTCCAACAAATGCACGTCCCCGCCTTCCACCCAG |
| | | GCGAGAACGCACCGAAGGCCTACGCAAAC |
| 59 | 632327_266823 | ACGCAAACTCCTCCACTTCTGGGAAGGCTGCCCGGTTTGCCTTCTTC |
| | 63_Target4.4_2 | CTCCAGAGCCGAGACATGGCCTCAAGTCGACTTGTCTTTCCCGCTAA |
| | | CCAACCCCAGGGCATTCATCGCGTGT |
| 60 | 632327_266823 | GGTGGGACCGTCTACAGTCGGAGCTGTGGTGGTGATGATTCGTCTC |
| | 63_Target4.4_3 | CTACAGGCTGGGGCACGCCCCCTCCCCTTTCCCTCTAGATTCAGATT |
| | | TGAGTCCCACGGCCCATGGACACGCGA |
| 61 | 632327_266823 | TCCCCAGATGTCGGCACAATGCAGGGGAAGGTAGGTGCTGCCCGTG |
| | 63_Target4.4_4 | AAAAGCGCTGGGTCGAGAGCTCGCCAGCCTCCCTGCTTGAGGCTTC |
| | | ATTTCCTGGGGTCCTTACACGGTGGGAC |
| 62 | 632327_266823 | GCTGAAGCAGATGAGTTGGAGCAGGACTCAGGAGGGTACTAGGCCC |
| | 63_Target4.4_5 | AGGTTTTAGGCTGAACCTAGGAATGAGTAATGAGAGGAGGCAGATCA |
| | | GCTGGAGGACCAGCCCGAGTCCCCAGA |
| 63 | 632327_266823 | ACCGAGCAAGGGGCAACTCAGGAGAGCCAAGGTTGTCTCCACTCGG |
| | 63_Target4.4_6 | TTTCCCCCGAGTGATTCTTCCTTCTATCTCCCTCATTCCCCCAGTGCC |
| | | GCTCTCCTACCCCATGTCGCTGAAGC |
| 64 | 632327_266823 | TGCTCGGTTGCAGGTCAGTGACTGTCACCCCATGCAGCAGAACCAG |
| | 63_Target4.4_7 | GGTGCCAGAGCCCCTGGGCCAGGCCAGGGAGGCACAATCTGCAGG |
| | | GAGATGATAGGACCGAGCCTGGTCCAGAC |
| 65 | 632327_266823 | CTCTGCTGCGTGAAGCCGGGTCTACCCCGCTGCTTCCCTGCCACCA |
| | 63_Target4.4_8 | GGGCTGCCTCGTGGCATCCCATTGCTCAGCCAGTGGCCCAGGATTG |
| | | TTGACTGAGGGTCCTGTCTGGTCTGGAC |
| 66 | 632327_266823 | CAGCAGAGAATTCTCAAGCATGGCCTATGGAAGACACAAGCCCAGGT |
| | 63_Target4.4_9 | GGGCCTGCAGCTGCCCCTCTGTCTGACCCCCAGGGCTCAGCCTACG |
| | | CAGCGCCTGGCCAGATGTGCCACCTTG |
| 67 | 632327_266823 | AGACTCCTGTGTCCCACCCCTCCCAAAAAACTGGACGTCTGCATTTC |
| | 64_Target4.5_1 | TTCAATATTTGGCTTGGGAAGCATTGGCCAGAACTCACAGCACAGCG |
| | | GGTGAGGAAGGAGCAGTGGGATGAGA |
| 68 | 632327_266823 | GACAGAGCATGACCCAGAGTCCTCAGGAGGAACAGAACAGCTAGCC |
| | 64_Target4.5_2 | AACACCCTGTGCCTCCCCGACACAGCCCGCCTCGCCTCAGAAGAGG |
| | | AGCCAGCCTTCAGGGAAGCTGCCTGGAG |
| 69 | 632327_266823 | AGCATCTCCTCCTGCTTGTTCCCAAGAGCTGTGCCGGGCCCCTCCTG |
| | 64_Target4.5_3 | CACCCTGACCCTGTCCAGCCACCCACTCCTCAGCCTGGCTGGCACA |
| | | CCAGCTTCCCAGAGAGCAGGTCTGAAC |
| 70 | 632327_266823 | GCAGAGACTGCTGCCTGGGGCCCTGCCAGCAGGGCACTGCCTGCC |
| | 64_Target4.5_4 | CATGAGGCCTGGTCTCCCCTCCCCACAGAAGTGGAGGCCCAGGGAT |
| | | GACCTCAGGCTCCTGAGGGTCCCGCTGGT |
| 71 | 632327_266823 | TCATCCCCCAAGGTTCCAGCCATCCACCTCCTCTCTGCACCGCCCTC |
| | 64_Target4.5_5 | CTTGGCCAACTAGGATGGAGCAGGGGCCAGCTCCCTTGAGGGCCAG |
| | | GCCTTTTGTGTCCTTGCCCGTCCCTGC |
| 72 | 632327_266823 | AAAAGGAGCAAATTTTTGAAATGCCTGTGTCGGAGCCTGTTTCCCTTG |
| | 64_Target4.5_6 | CTGCCTCCTGCTCACACCAGCAGCCCCACCCCCAGTGCTGTTCTAAT |
| | | GCAGCTACTGGGGGAGGAGGGTCTC |
| 73 | 632327_266823 | CCCCACCCCCCAGCTACATCCCCATCAACACTGTGGGGCTTAACTTC |
| | 64_Target4.5_7 | TGGGGCCTTGTTTCCAAAGCGGAAATGCCATTCTGGGAGGCACAGG |
| | | CTTTTGGGGGAGGGAATTTAAGTGGAA |
| 74 | 632327_266823 | GGGTATGTGGGGCTGCATCCCTGTCCTTGACAGCGGAGGTCCGGGA |
| | 64_Target4.5_8 | GGCTGGACTGGGGGTGGGGTGAGGTTCTGCCCCCATATACCCCGGC |
| | | CAGAGCCTGGCCAGTGGGTGCTCCTCCC |
| 75 | 632327_266823 | TCTCAAAAAAAAAAAAAATTGGGGCTAACGGTGAGCCTGTTTTGCAG |
| | 64_Target4.5_9 | GATTGCTGTGTGCACAAAATAAATGCATGGGCATAGGGCCCCTGGCA |
| | | CTGGCTGGGCGCAGAGGCTGTGCTGG |
| 76 | 632327_266823 | TTTCTTCCCTGGCCCTGTCCCTTCTGTGCCCCGAAATGTGTGGGATG |
| | 65_Target4.6_1 | GGGCAGTGGTTGGTAAACTGGGTCTCGGTCTCTTCATGTGAAAAATG |
| | | GGGCTAGGCTGGGTGCAGTGGCTCAC |
| 77 | 632327_266823 | GCCTGTCCTTTTGCAGTCTGAGTGTGACAGGGGTGTTTGGCTTTCTG |
| | 65_Target4.6_2 | AGCCTGGAGTGCTGCTGTCCCTGTCCCTAGATTCCCTTCCTGCTGCA |
| | | GGGTGGGCTGAGGTCTAGCAGCCCCA |
| 73 | 632327_266823 | TCAACAAATGTTTACTGCACCCCTTCTCACCAATGGGGCTAAACTTAG |
| | 65_Target4.6_3 | TGGGGTGGGGGTGGTGCTGGCCCCAGTGCTGAGCTCAGTTTTACTG |
| | | GGGAGGAAGGTGCTTAGGTCTCAGTT |
| 79 | 632327_266823 | GTGTGTGCCTCTGCACTTAGGCGTGACTTAACCACCCCCATCCTCCA |
| | 65_Target4.6_4 | GACTGGGGCAGCCTCAGATTTCTTCACCCAGGCCAGCCTATCCCATG |
| | | AGGGCCACCTCTGAGGTCCCACCCAG |
| 80 | 632327_266823 | GACCATTGAGGAGAGCTGGTGGTCACTCTGCAGAGCGTCCCTCCGT |
| | 66_Target1.1_1 | GGGGATGGTCCTGGGTGGCCTCAGGACTCAATGGAGGGAGTCCCG |
| | | GGCGTGGCGTGTGTCTGTCTCAGGCCCCC |
| 81 | 632327_266823 | GCCCCCTGAGTATCACCAGCCACCCTTTGCTGGGTCCTGGGTACACA |
| | 66_Target1.1_2 | AGCCACCCATCTTCAGATGTCAGGGTCTCCTCCAGCGGGCGCAGTC |
| | | CCTCTGCCCTCCCTCAGCCCTTGGGAC |
| 82 | 632327_266823 | TCCCTCCCCCGGCCTCCCTCCCCCAGCTTCCCTAAATTCTGTCCTCT |
| | 66_Target1.1_3 | TGCTCAACCCTCCAGGCTGTGATCGAAGCCAGGGCGCGAAAGTTGA |
| | | TTCCAAATGGCCCCCTCTGCCCCAGCC |
| 83 | 632327_266823 | AGAGAAGCACTGAATGAACTTGGAGGCCAAAAAGAAAAAAAGCTTTT |
| | 67_Target1.2_1 | TAACGTTTCATTTGGAAAGGATTTCACGCTTACAGAAGTCCAAAGGGT |
| | | CCAAACTCCCTCCCCGCTCCCCAAA |
| 84 | 632327_266823 | TTTTCTTTTTGGCCTCCAAGTTCATTCAGTGCTTCTCTCCATTTCACTG |
| | 67_Target1.2_2 | TAGGTTTAAACCGTTCAGTTATAAATAAATAATTTTATCCTCTGTCCTC |
| | | GGCTTTCCCCCGTTTCCGGCAC |
| 85 | 632327_266823 | GACTTGGAGGGAGCTCCTGGCAGGGCCCTGCAGTGCCCAGGATGG |
| | 67_Target1.2_3 | CCCCGCCGCAGAGACAAATCCAGCCCCCATGTCTGCAGTGCCGGAA |
| | | ACGGGGGAAAGCCGAGGACAGAGGATAAA |
| 86 | 632327_266823 | AGAATCACCCCAGGGTTAGAGGATTCCACAGACCCCAAGAGACTCTG |
| | 68_Target1.3_1 | AGTCCCATGATCCTCTGCCCTGCTGAGGTCCCAGGAGGCTGCACAC |
| | | ATTGAAATCCACACCCTGAGTGGGAGG |
| 87 | 632327_266823 | TCCCGATGGGGCGTACGGCATAAACCCGGGGGACCCCAGCTCCCG |
| | 68_Target1.3_2 | GGACTCGCCTCCTCTCTCCCTCCCACTCAGGGTGTGGATTTCAATGT |
| | | GTGCAGCCTCCTGGGACCTCAGCAGGGC |
| 88 | 632327_266823 | AAATAAAAATAAAAAATAAAAAATCCAGTCTGCCCCCTGCTCACACCG |
| | 68_Target1.3_3 | GACACCAGAATAAACTCCCGATGGGGCGTACGGCATAAACCCGGGG |
| | | GACCCCAGCTCCCGGGACTCGCCTCC |
| 89 | 632327_266823 | CAGGGCCCCACCACGAGTGCCCTCAGTGCCTCACCTGAGGCCCCAG |
| | 69_Target1.4_1 | CGAACATCCTCCTGCCAGTTTTGTCTGGGGGTTTTCTTCCCCCTTTAA |
| | | GGCCAGGATTCTCAATCAAGAGGTGA |
| 90 | 632327_266823 | GAGGTCCCTGTGGCCCCAGCAAAGCCGGCCGTGGCAGGTGTGTCC |
| | 69_Target1.4_2 | CGTGGGTGGGTGAGCTGGAAACGGACAGACAGCAGGACGTAGCAG |
| | | GGGACAGGATGCAGAGCTCGGGAAGGCAGG |
| 91 | 632327_266823 | CCTCGTCAGGGAGGCGTCTCCTTGTGGGTGGCACGTGGGCATGTGT |
| | 69_Target1.4_3 | TGCTCTGAGGGCTGTGGCCATGTTGCCCACCTGGCCAGGGACCCCC |
| | | GACTTGGGTGGGTGACAGCCAGCCTCCC |
| 92 | 632327_266823 | CAATGCCCAGGCTGGGTCCCCACTTCCTCCTTTTGCCTTGGGAGCCT |
| | 69_Target1.4_4 | GGGAGTCAGAACGGACTCCGGGGAGGAGGTGGGGGCTTTGGGCTC |
| | | AAGGTCCCAACCTTTGTGGGGGCGGGGA |
| 93 | 632327_266823 | ATTGGGCAACTCAACGGCCTCTGGCATTGGGCTATAAGAGGAGCTTG |
| | 69_Target1.4_5 | ACCGTGGGTGCACCCTGGACCCCACCATGGCTCACCGGCCCCCCAG |
| | | CCCTGCCCTGGCGTCCGTGCTGCTGGC |
| 94 | 632327_266823 | CCCAGCTGTGCCAGAAGGGCTTTGGATGGACCTCGTGGTGGATATTT |
| | 69_Target1.4_6 | CCCCACAATCCACCGGGGCCTGGAGGCTGGATGGATGGGCACGTG |
| | | GCTCACTCACCGCTCAGCAGCAAGGCCA |
| 95 | 632327_266823 | ACAGATAGGCAACTGAGTCAAATGGCCAGGGCTCCCCCTTGCCCTTG |
| | 69_Target1.4_7 | TCCTCCCCACCCCTGCAAGCGTTGGTCCCGAGCAGTGAGTGGAGTC |
| | | TCCCCTGACCCTGCCTCAGTTTCCCCA |
| 96 | 632327_266823 | GCAAATGCTCAGTGAATATGCATGAATGAATGAATGAGTGAATGAATG |
| | 70_Target1.5_1 | AGTGAGTGAGTGAATGAGTGAACAAATGAATGAGTGAATGAATCAAT |
| | | GAGTGAGTGAATGAATGAGTGAATG |
| 97 | 632327_266823 | AGTGAATGAGTGAACGAATGAATGAGTGAATGAATCAATGAGTGAGT |
| | 70_Target1.5_2 | GAATGAATGAGTGAATGAGTGAATGAATGAGTGAATGAGTGAATGAG |
| | | TGAACGAATGAATGAGTGAATGAATG |
| 98 | 632327_266823 | AAAAAAGAAAAAGAAAAAGAAAATCACCCTCAGGATAGGGACCTGAG |
| | 70_Target1.5_3 | GGGGCCCATGCTCTCTTCTTCCCCTGATTCCAGCATCACTAACTCCT |
| | | CGACCCTTTCACTCACTCACTCATT |
| 99 | 632327_266823 | GTGAGCCACTGCACCCAGCCACCTGGTCTGCGTCTTAAAAGCCTTCC |
| | 71_Target1.6_1 | TGACTCTCAGGACTGAAAGCTGCCACCAGGGCGCCTTTGGAAATCGT |
| | | CGTAATTATAACCCCCCCGGCCTGGG |
| 100 | 632327_266823 | GGGAGCCCCCAGGCTGCAGGGAAAGGGGGGATGGCAGAGCAGCCC |
| | 71_Target1.6_2 | CTGGGCCTCTCCGAGCCTCCGTCTCCCGATCTGCAACCCGGTCAGT |
| | | GCCTGCTGGCTGGTGGGAAGGACTCAGCG |
| 101 | 632327_266823 | GCCCGGGTTCCCCCGCATCTGCAGGGAGGCCATGTAGGGCCGGGA |
| | 71_Target1.6_3 | GTGTGGCTGCGCCTCGTGCCCGCCCACGATCTCCGCAGCTCGGGCA |
| | | GCACCTGCAGGGGGGGAGTCCAGGCGTCA |
| 102 | 632327_266823 | AGCCACTTCTGCGGAGGCACCTTGATCCACCCCAGCTTCGTGCTGAC |
| | 71_Target1.6_4 | GGCCGCGCACTGCCTGCGGGACATGTGAGCGGCCGCCTCCACACC |
| | | CCTGTCCGCCCGCCCCGCCCTCTTCCTC |
| 103 | 632327_266823 | CTCGGCGACCCTGCCCCGGGCCTCCCCCCAGAACTGCCCACGCGC |
| | 71_Target1.6_5 | GCGGGGCCTGGGGTCTGCAGACGGGGCTTAGCTGGGTCCTCCCCG |
| | | GGCAGAGGGACAGTGGCCGGGCCAGGGCTG |
| 104 | 632327_266823 | CCACGTTCACCAGGCGCTGGGGTCTGCGGGGGTGGGGTGGTCACT |
| | 71_Target1.6_6 | CCTCGGCGCCCTGGACACTCGCCGCCGCCCACAGCCGGCCCTTCC |
| | | CGAGGCCGCGGTGCAGCCCCAGACCCCTCC |
| 105 | 632327_266823 | TGCTCGGAGCCCACAACGTGCGGACGCAGGAGCCCACCCAGCAGC |
| | 71_Target1.6_7 | ACTTCTCGGTGGCTCAGGTGTTTCTGAACAACTACGACGCGGAGAAC |
| | | AAACTGAACGACGTTCTCCTCATCCAGG |
| 106 | 632327_266823 | CCATTTCTGTCTCCATAGAGTGGGGACAGCCCCCCATTTCCCCATGA |
| | 72_Target7.1_1 | CACACATGAGATACTAAAGAGGTGCCCTACAAAGTCCCATCACCACT |
| | | AGATGATTTTTTTTTTTTTTTTTTTG |
| 107 | 632327_266823 | GAGTTGGGGCTCCGCGGTGGCGGTCAGCAGCATAGGCCCAGGTCC |
| | 72_Target7.1_2 | CATGTGACCCAAGGCTGTCTCTGAACCTCCATTTCTGTCTCCATAGA |
| | | GTGGGGACAGCCCCCCATTTCCCCATGA |
| 108 | 632327_266823 | TCCCCCCCTTATGACCTGTAAACCCAGTTTCTGGCGGCCAAGGATGG |
| | 72_Target7.1_3 | GGAGACAGGAGGCAACTGCTTGAGCGGAGTTGGGGCTCCGCGGTG |
| | | GCGGTCAGCAGCATAGGCCCAGGTCCCA |
| 109 | 632327_266823 | CTGCCCCTCCACTCCCGACTATGAGAACATGTTTGTGGGCCAGCCAG |
| | 73_Target7.2_1 | CAGCCGAGCACCAGTGGGATGAACAAGGGTAAGTAAGCCACTGAGA |
| | | CACCTGCCAAAGTGGTGAATAATAAGG |
| 110 | 632327_266823 | CCGGGAGCTGAACAAACCCTGGGCTGCTCAGGATGGGCCCAAGCCC |
| | 73_Target7.2_2 | GGTTTAGGCTTGCAGCCACGGTACGGCAGCCGGAGCGCCCCCAAGC |
| | | CCCAAGTGGCCGTGCCATCCTGCCCCTC |
| 111 | 632327_266823 | AGCTCCCGGCTTCTGGCCACTCGGCATCGCCAGAGTCTCCAGGCCA |
| | 73_Target7.2_3 | GCACAGGGCCAGCGATGGCAAGTCCAAGAAGCAGGCACCCGCTGAC |
| | | CACCACTGCCCCGATAGTTGCAGAGGCC |
| 112 | 632327_266823 | CGACATCCGCCGAGACAACTGCTCTGGCCAGAAGCCTCTGCTCTGC |
| | 73_Target7.2_4 | TGGGACACAACCAGCTCCCAGCACAACCTCTCTGCCTTCCTGGAGGT |
| | | CAGCTGCGCCCCTGGCCTGGCCTCTGC |
| 113 | 632327_266823 | TGAGGTCCTGAACGTGCTACGCAACCCCTTGTCTCGTGTGGATGGG |
| | 73_Target7.2_5 | GCGCTGGCCGCCCGCTGTGACCTTGACCTGCAGGCCGACTGCAACT |
| | | GTGCCCTGGAGTCCTGGCACGACATCCG |
| 114 | 632327_266823 | AGGACCTCAAGCTTCTGCAGGTGGGCAAAGAAGGTCACTGGAAGCT |
| | 73_Target7.2_6 | CTCGCAGGCCGTTCCCAGACAGGTCAAGGAGAATCACGTTGCTGGC |
| | | CCGCAGAGACTGGTTGTGAGGCAGGCTC |
| 115 | 632327_266823 | GGCAGGCTCAGGCTGAGGCCACTGAAATTCAGGCACGTGGCACTGA |
| | 73_Target7.2_7 | ACTCCGCGTTCCAGTCCACATCCGCGGAGGACACGGTGCACGACGG |
| | | TTCTAGGCTGTCTGACTCCCGCAGCAGC |
| 116 | 632327_266823 | CGCAGCAGCAGCGGCAACAGCAGCGTCCATGCCAGGGTGCCCCCC |
| | 73_Target7.2_8 | ATTCAAGCAACCTACGTAGAGACACCGGAATCCTAGCCCTGACCTCA |
| | | GCCCTGTGGTCGCCCTCGCCTCCACCGC |
| 117 | 632327_266823 | CTCCACCGCCAGTGTTTATTATTATAGCTCAGACCAGCTCAAGCCGC |
| | 73_Target7.2_9 | TTCTGAAATGGTAAAACGCAGCCCCAGTCTGGTCGCCTCCTTTGGCT |
| | | GGTGGGCTGCCTCAGTCTCCCCTTCT |
| 118 | 632327_266823 | GCATAGGGTCCCGTCCTGGCCAACGAGGGCGCCCCAAATGTTCAGG |
| | 74_Target7.3_1 | ACATAGAAGAAGGGGTTAACTGGCCCGGATCTCCTCCTCGCCTTCCA |
| | | AGCCCGCTAAGCACTGGGGTTATCTAC |
| 119 | 632327_266823 | GCCCCCACTCCACCCTATCCCAGGACTTCCCAGCGACCCGCCGTTC |
| | 74_Target7.3_2 | TGGGAGATACCGGGAGCGTGATCAGGGGGCGGGGCCGTTTCCAAG |
| | | GCAACCGCTTATTTGCATAGGGTCCCGTC |
| 120 | 632327_266823 | GCAGAGGCAAAAGCGTGGCAGTGGGACCCAAAAGGTAGGACTGAGG |
| | 74_Target7.3_3 | CTCTAGAACTTGCACCTGTGCAGGGACTGCAAACCAGACCTGGGAG |
| | | GACCCTTTCAGCAGCCCCCACTCCACCC |
| 121 | 632327_266823 | AGCGACCCCAGCAGCGCTGCGGACGGTGCTGGCCGTGGCCGCTGC |
| | 74_Target7.3_4 | GGCCCCCGTGTCCAGGTGGGCCAGGACGCAGCCTCTGGGCGCCGT |
| | | CGCTTTTCCAGCATCGCAGAGGCAAAAGCG |
| 122 | 632327_266823 | CCTGCAAGGGGGCGGATCTCTGTCTTCATTCTTCCCCTCTGCACTCC |
| | 74_Target7.3_5 | ACCCAGACACCAGTTCCTCTTCCCCCTGAGTCGCTGGGTGCAGAGC |
| | | CAGAGGAACGCCAGCGACCCCAGCAGC |
| 123 | 632327_266823 | GCTAATAGAGGACATGAAGTCACTGGGTTCTAGCAAGAGGGAAGCG |
| | 74_Target7.3_6 | GGACTCTGGTCCACCCCTCCCTCTTGCCCTCCATCCACCTCTGGTGA |
| | | GAACCTTGAGGCCCTGCAAGGGGGCGG |
| 124 | 632327_266823 | ATGTCCTCTATTAGCACCCCAACCGACTGCCACCTCATCAACTCGCTT |
| | 74_Target7.3_7 | CTTGATCTCAGCTTGGCTGGTTCCCAGCTCATGCAGTCATTTATTCAT |
| | | TCAACAAGCATCACCCACGCTGGG |
| 125 | 632327_266823 | CCCTCCTCACTGTCCCCTCCAGCATAGAGGTGCCACTGTGCAAGAAC |
| | 75_Target7.4_1 | TCAGTGGGAGAGGCTTGGAGGAGCTTGGAGAGCCAATGATAGGTTT |
| | | GGGGAATCTTAGTGAACTCCTACTCAT |
| 126 | 632327_266823 | GGCTCCACATGGACCTCTTCCCCCACCACCAATTCCTTCTATCCTCTC |
| | 75_Target7.4_2 | CACTCCTAACCATGCTATCCCAGCCAGCTACATTTTCCCAAGCTGCT |
| | | GGGAGCCCTCCTCACTGTCCCCTCC |
| 127 | 632327_266823 | CTGGGACCTCAGCCTCCTCCCCAAGTTGCCCACCCCAGACACCAGG |
| | 75_Target7.4_3 | CTCACTCCAGAGGGACTTCTTGAGCCAGTGCCCCTCTGCCCACGCA |
| | | CCTGCCTTGGCTCCACATGGACCTCTTC |
| 128 | 632327_266823 | TGACACCAGGTTCAGGTGAGGCCTTCTCCCAGCCCACCCCTCCTCA |
| | 75_Target7.4_4 | GTATCATTTTTGTCACTACTACCCCTCCAGTCTGCCTCAGGCCCCCAT |
| | | CTGCTTCTGGGACCTCAGCCTCCTCC |
| 129 | 632327_266823 | CTCACCTGAACCTGGTGTCAGGTGGACCAGGTACAACCTATGCCAGC |
| | 75_Target7.4_5 | CCGACTCACTGTGCCACCTTAACTGCGTGTGACACCATGTCCACAGC |
| | | TGTCAAATGCACAGAGGCTTCTCCGG |
| 130 | 632327_266823 | ATGCACAGAGGCTTCTCCGGGATCTGGGAGGCTGGGAGTCCACTCC |
| | 75_Target7.4_6 | CCCACGTTTTCCCCTCTTTTCGGCCTCTGTCCTTCACATCGCCAAACT |
| | | CCAAGAAATGGTCAAAGGCTTTGAA |
| 131 | 632327_266823 | CGTGCTTTGTTCTACTGGCCCTGTTAAAACTCAAGCCAGATCACGTC |
| | 76_Target3.1_1 | CACCTGCTCACAGCCCTCCTGTGGCTCCTCCTCACTCTCAGCAGAAA |
| | | CTGACCTTCCCCCTCTTATCTCACCT |
| 132 | 632327_266823 | TCTGCTGTCTCCAGGTGGCGGCGAGGGAAAGCGCATCCTGGGGGAA |
| | 76_Target3.1_2 | CCCTGGGCGCTTCCTCGCGTTAGACTCCCCCAGGTTGCCAACTGGC |
| | | CAGTGGCTGGTTTCGGTACCCAGGCGTG |
| 133 | 632327_266823 | CAGAGTCACGCCCAGCGCTGCGCAGGCTGATCGCCGCGCCGCGCC |
| | 76_Target3.1_3 | CCCGCCCTCGGTCGCAGGTGGCTCGTTCCGGGAATTCCTAAGCGGA |
| | | AACCGGTCCCAAGCCCCGCGCCTTCGCTC |
| 134 | 632327_266823 | ATTCCACTTCGGAAGTTTTCGCCACCTCCCCCACCCGTTCCCAACTT |
| | 76_Target3.1_4 | GTGGTTCGGCCCCTGGGCATCCCTAGCCCCCGGGTCAGCCCTCCGG |
| | | AAATTCTGGCTCTTAAAGGGGCCGAGC |
| 135 | 632327_266823 | TTCGGGCCTCCTGTGTCTCCCAGTTAGATGACCTGAGGAACCCTCTA |
| | 76_Target3.1_5 | AAGTCCCCGGAAGGGAAGCTCTGCCCCCTCAGGGATGCCCAAGGGG |
| | | AATCAGGGCCAGGAAAAGTTGGAATTC |
| 136 | 632327_266823 | CGAAGCGCCCCCCCTCCACCCGGTCCGGAGGAACCCCAGTGGAAGT |
| | 76_Target3.1_6 | GGAGAAGTCAGGCGCCACCAACAAGCCTCTCCCAGCCAGGACTTTG |
| | | CTTAGACTCGCTCCTCCCGGCAGGGCGC |
| 137 | 632327_266823 | TGCAACCGAGACCCACTTGGAGCGATTCACCTGTTGAGATAGGGCG |
| | 76_Target3.1_7 | GGTCCCGCCGCGCACCTGTTCCCTCCCTGCCCAAACCCCTCTCCCC |
| | | GGCTGGCGATGGACCCGCCTAGGTGCGC |
| 138 | 632327_266823 | GCGCTCCGTCCCCCAGGGCCCGAACCAAGTGACTGGCCCGCTGGG |
| | 76_Target3.1_8 | AGGGCGCCTGCGGACCACAGGTCCCTTCCGGCCCCCATCTGCTCCG |
| | | GCCAAACCAAGCGCACCGAGATCCATGCA |
| 139 | 632327_266823 | CTCCGGTGAATGGTTCGGCCCAGGTACACAAGGGTGGGAGCCGAGG |
| | 76_Target3.1_9 | GCTGGGCTTAGGGGTGAGCGGCGGGGAGGGGTTCCTCTGCCTCCC |
| | | TCCCCGGAAGTGGGCTGACCCAGGTGCGC |
| 140 | 632327_266823 | GGAGCGCGCAGCGGGTGGAGTGTGGCTCGGAGGACCGCGGCGGG |
| | 76_Target3.1_10 | TCAAGCACCTTTCTCCCCCATATCTGAAAGCATGCCCTTTGTCCACGT |
| | | CGTTTACGCTCATTAAAACTTCCAGAAT |
| 141 | 632327_266823 | GCGCGTCCCTTCCAAAGTACCTTAGGGGGCGCGGCGCGCACCAAGG |
| | 76_Target3.1_11 | CCAGGAGGGGTGCACGCTCCTCCCCTTCCCACTTCCGTTCCTTGTCC |
| | | CTACTCCAAGTCCGTCCTGTTGCATTC |
| 142 | 632327_266823 | GCGCGGGCCAGACGCGCCCAGACGGCCGCGATGGCGCTGTTGGCC |
| | 76_Target3.1_12 | GGCGGGCTCTCCAGAGGGCTGGGCTCCCACCCGGCCGCCGCAGGC |
| | | CGGGACGCGGTCGTCTTCGTGTGGCTTCTG |
| 143 | 632327_266823 | ACCCCCGCTTCCCGCTTCCCGCCGCCTCCAACTCCATCCATCCCCCT |
| | 76_Target3.1_13 | CTACAGTTCCCTCCGGCGTTCCGCAGCGTCAGAGGCCCCGTGCCCC |
| | | GTACCTGTGCACCAGGTGCTAAGCAGA |
| 144 | 632327_266823 | TATCTTCCAGATAAGGGCGCGGGGAGCGCTCCCCGGCACAAATGGG |
| | 76_Target3.1_14 | GAATTCCTCTCACTCCCGCGCGCAACAGATCGCCCAAGACCCAGGG |
| | | GATCGGAAGGTCCCAGCCTCTGAGACCC |
| 145 | 632327_266823 | TCCCATCCCGGCTTTTCTTTTCCCAGAGATCGGGCGGTCTTCCACTC |
| | 76_Target3.1_15 | CCTCCCCACAAAGAGTTCCATTCTCCCTGCCGCTCCGGGTCTCACCG |
| | | TCCAGGGAGTTTCACTTCCTGCTATC |
| 146 | 632327_266823 | ACCCGAGGCCTAGGCATTGTTCCTTCGTCTCTGTCCCTCTAGGAAGC |
| | 76_Target3.1_16 | GTCTGATCCAGCCCTCCCGTGCCCTCTCCCCGTTACTCCAAAACTTA |
| | | GAAATCCCCGGGTGCGCCCAAGTCCC |
| 147 | 632327_266823 | CAGAAGTCGGTTTCAAGTCCGTAAATCCATCCCACCAGACTCTTACG |
| | 76_Target3.1_17 | CACGTATAATCCGGAGCCCGCGGGAGGTGGGTGGGGTGCGGGGGA |
| | | TGGGGAGTCCCCAGTCCCACACCCACCC |
| 148 | 632327_266823 | TCTGCTGGCAGGCTTTCACCTGGATGGGATATTTGGGTGGTGATGAG |
| | 76_Target3.1_18 | GTCTTTCCCGAGACACTTTTGGTTCAGTCATTTGAAATGACTTTAGAG |
| | | TAGGGTGAGGTGGTGGGAGGCTGAT |
| 149 | 632327_266823 | ACCGACTACGAAGGATTCCAGGTTACCCGGGGCCTTTTGAATTTGGA |
| | 76_Target3.1_19 | GGGAAAACCATGGAGTTGTTTGCCTGAACTGCTTTGCCATGGAGGGA |
| | | CTAAAGCCCCCACAATATCTCCATCA |
| 150 | 632327_266823 | CACCAGTCCCCTCTTCCCTTTGCCATTCACCCACAACAAGGGGCCTC |
| | 76_Target3.1_20 | TGTGTGGCCTGGGCAGGCAAGCCCAGATGGCCATGTTGATGCCCCC |
| | | AGCGCCCAAGGCCCCACTTCAAAACCG |
| 151 | 632327_266823 | GGTGTGGTTCAGAGGCCACAGGCTGGGAAGAGGGATGGCGGGCGA |
| | 76_Target3.1_21 | GTCCAAGGAAACTGGCCGTGTCACCGTGCACCTGCCACTTCAGCCC |
| | | CACGGGTCTATAAAATGGGCATGATTAT |
| 152 | 632327_266823 | GTGCCAGGCACTCTGTAAACCACATACTTGCGAGTGTCAAGCTGGTG |
| | 77_Target3.2_1 | ACAGGTGGCGTTCCTGTTGAAGCACCTCCCTGAGCTCACAGCAACCC |
| | | TTGCTGTCTCTCCTCTTGCCCTCAGC |
| 153 | 632327_266823 | TCTCTCCTCTTGCCCTCAGCTCCTGCCAGGGCCATCCAGGTGACCGT |
| | 77_Target3.2_2 | GTCCAACCCCTACCACGTGGTGATCCTCTTCCAGCCTGTGACCCTGC |
| | | CCTGTACCTACCAGATGACCTCGACC |
| 154 | 632327_266823 | CCTACCAGATGACCTCGACCCCCACGCAACCCATCGTCATCTGGAAG |
| | 77_Target3.2_3 | TACAAGTCTTTCTGCCGGGACCGCATCGCCGATGCCTTCTCCCCGGC |
| | | CAGCGTCGACAACCAGCTCAATGCCC |
| 155 | 632327_266823 | CGACAACCAGCTCAATGCCCAGCTGGCAGCCGGGAACCCAGGCTAC |
| | 77_Target3.2_4 | AACCCCTACGTTGAGTGCCAGGACAGCGTGCGCACCGTCAGGGTCG |
| | | TGGCCACCAAGCAGGGCAACGCTGTGAC |
| 156 | 632327_266823 | GCCAGTCCCACCACCCGGGCAAGCCCAGCCTCATCCCCCTGCCCTG |
| | 77_Target3.2_5 | CCCAACATACTTCCGGTGATGGTAATCCTCCGGCCCTGGTAGTAATC |
| | | TCCCAGGGTCACAGCGTTGCCCTGCTT |
| 157 | 632327_266823 | GCCCGGGTGGTGGGACTGGCGTCCTTGTGCGGGACCTGGAGTCCC |
| | 77_Target3.2_6 | CATCTGAAAGCTCTTGAGTGCCAGTGTCTGAAAGGACCATTGAAGGG |
| | | AGCAATTCTTTTTTTTTTTTTTTTTGA |
| 158 | 632327_266823 | GTGTGCAGTTCCTGGCATGTCACAGGCTTTCTCCATTAAGCTCTCTTG |
| | 78_Target3.3_1 | CCCCTGCAGTTCACTCCTGCCATCTCCTCTGTGAGTCCTCTGCGGAC |
| | | CACCCTAGGCAAGCAAAGGGATTAG |
| 159 | 632327_266823 | GTAACCTCTCTGTGCCTCAGCTGTCAGGAGGGTAGAGGAGAGGACT |
| | 78_Target3.3_2 | CAATGGGAGAATATGTGTGCAGTTCCTGGCATGTCACAGGCTTTCTC |
| | | CATTAAGCTCTCTTGCCCCTGCAGTTC |
| 160 | 632327_266823 | CCTCCCAAAGTGCTGGGATTTGTTTCCGGTTTTTTTCCTACTATGCTA |
| | 79_Target6.1_1 | TAGTTTTGACTTTGTTTCCCTGACTAGGATGTCAATTCCGTGAGGCTG |
| | | GATTTTTGCTGATTTTTTTTTTTA |
| 161 | 632327_266823 | ACTATGCTATACTTTTGACTTTGTTTCCCTGACTAGGATGTCAATTCC |
| | 79_Target6.1_2 | GTGAGGCTGGATTTTTGCTGATTTTTTTTTTTAACCTCTGTACCCCTAG |
| | | CTTTTTTTTTTTTTTTTTTTTTT |
| 162 | 632327_266823 | GCTTCCTCCGCGGCGCTTTAACACGCAGGGCGCTGCTGCCAGGGGC |
| | 80_Target6.2_1 | GTCCCGTGTTCTAACTCGCTCCCACAGCCCCTCCGGCTTGGTGAGCA |
| | | GCGTCTGAGGGGTGAGGGGCATAGACT |
| 163 | 632327_266823 | GGGGGCTCTGTGGATGGCATTCGGGGAGCTACAGGTTTCCCCCAAA |
| | 80_Target6.2_2 | AGCTCAGATGCTCGTTCTTGAAGAGGGAGGTGCTGCCCCTGCCTTCC |
| | | TGCGTACCGCGACAATACAGCTTCCTC |
| 164 | 632327_266823 | GAGCCCCCCTCAGTCGGCCTCCCCTGAGGCACCAAGGCTGGCGGAA |
| | 80_Target6.2_3 | GCAGTCACCTGTCCATCTCCCCCACTTCTCACAGAGGAAATCCTGGG |
| | | TGGTGCCGGGCAATGAGTCAGGGCTGA |
| 165 | 632327_266823 | CCTAAGGCCAAGGGCGCCTGGGCCCTGCAGAGGAGCGGAGCAGGG |
| | 80_Target6.2_4 | GGAGGAGCGCTGAGACCTGCCCGTTGGAGGAATGCTGAGACGCCC |
| | | CACCCAACCTCTGTCCTGGTCCTCAGCCCT |
| 166 | 632327_266823 | TTGGTGCAGGCAGCTTCTGGGCTTGAGTCCGGCCCCCTGCACCTCC |
| | 80_Target6.2_5 | AGTCCACACTCCCCAGGAGCTCACCTGCTCCCAGGTCGAACTCCATG |
| | | GCGGTAAGAGAAGTTGGGTCCTAAGGC |
| 167 | 632327_266823 | TCCGACCGGCCCTGAACTTTGTGGGGACTGAGCTTGGGATCTCCCC |
| | 80_Target6.2_6 | CGTGGCCCGCCCCCACACCGGGCTTCTGGGAGGTGGGCTCCAGGG |
| | | CTGTGGAGAGAAGTTGGGTGGTTGGTGCA |
| 168 | 632327_266823 | TGAAGTTGGAGGCCTAAGGCAGGACCCTGGGGTCAGGGGCAACCCC |
| | 80_Target6.2_7 | AGCCTTCCCGCCCCTCCGCAGCCGGTGATGAGGCGACTTACCTTTG |
| | | GACCCGGACCTGCCCCTGCCTCCGACCG |
| 169 | 632327_266823 | TACCTTCACATCCGTGTCCGAATCGCTGGAGCTGCTGCTGGAGTCGG |
| | 80_Target6.2_8 | AAGAGCTGTGGTGTCCTTGCTGGATGGAGGTGCGGCAGTGAGGCGG |
| | | CGCCCCTTACCCAGCCCCCTGAAGTTG |
| 170 | 632327_266823 | GGCTGGAAGCGAGACAGGGGGACCACTCCCCGCACCCTCCCCGCC |
| | 80_Target6.2_9 | AGCCCCAGTGCGGGGACGCCTCTCTGGGGTGCAGGGCACGTGCTT |
| | | GGGGACGCTGGCGAGAGCCCCTTACCTTCA |
| 171 | 632327_266823 | TTCCAGCCCAGAACCATCTCTTCTCTCCCATCCCTGCCCTCGGCCCC |
| | 80_Target6.2_10 | ACAGTCCCACGCTGCTGGCTCCAAGCAGCACGAGAGCATCCCGGGC |
| | | AAGGCCAAGAAGCCCAAAGTGAAGAAG |
| 172 | 632327_266823 | AGAAGGGCAAGAAGAAGGAGGCTCCCCACTGAAGGGCCCTGGACAG |
| | 81_Target6.3_1 | GGCTCATTAAACCTTCCTCTCTGCCTTCTGCGACTGGTCAGCGTGGT |
| | | GCCTACTCTGGCCCGTCCCCAGCTCCC |
| 173 | 632327_266823 | CCAGCTCCCTGCCCCCTGCCCAGCGCTGGGCCCGCCCATTCGTGCG |
| | 81_Target6.3_2 | CGAGGCCAAGGAGAGGCTGTCCACGCCATGCCCATCAGGGTTTATT |
| | | GTTTCTGTAACAGCGGCCACGCCCTGGG |
| 174 | 632327_266823 | GGCCCCAAGGTCCACCCTGTCTGGCCACAGGCACCGCCTCCATCCC |
| | 81_Target6.3_3 | ATGTCCCGCCCAGCCCCGCCCCCAACCCAAGGTGCTGAGAGATCTC |
| | | CAGCTGCACAGGCCACCGCCCCAGGGCG |
| 175 | 632327_266823 | CATGTACTGGCTCACCTTCCACCTGGTGCGGTGAGCGCGCCCGCTG |
| | 81_Target6.3_4 | AACCTCCCGCTGCTGCTGCTGCTGCTGGGGGCCACTGTGGCCGCCG |
| | | AACTCATCTCCTGCCTGCAGGCCCCAAG |
| 176 | 632327_266823 | CAGTACATGAGCATAGGCTGCGCCGCCGCCACCGCCATGGTCAGGT |
| | 81_Target6.3_5 | ACATGCGCAGCTGGTTCCGGGCCCCACGCACCGGGACCCCCTCAGC |
| | | TGCTGCGTCTGCCAAGATCTTCAGCCGC |
| 177 | 632327_266823 | TTCAGCCGCAGCGTCCGGATCTGGGTGGGAAAGAGAGAGAGGAACC |
| | 81_Target6.3_6 | AAGGGATTTACCCTTGAGCCCTCTGGGCCAGAGTGAGAGCCCGGAC |
| | | ATACTTCCAGCCTCTGCCTAGAAGCCCC |
| 178 | 632327_266823 | AGGGCCCGGGGAGAAGCAAATCCTCACCATGTTAGGAGGTTTGGGG |
| | 81_Target6.3_7 | AAACTGAGGCCCCGGGAGCAGAAACCTGAGGCTGCAGAGGGCCAG |
| | | GGACTTGTCCCCAGCTGCCTGGGGCTTCT |
| 179 | 632327_266823 | GGGCCACGTCTCCCACCCCCTCAGGCCGGGTGTCCACCGGGAAGTC |
| | 81_Target6.3_8 | CCCCTTCACCCGCTCCAAGACCTTGCCTGGCATTTGAGTATGGAACC |
| | | TTCTCAGATTTTGCCCACAGGGCCCGG |
| 180 | 632327_266823 | ACGTGGCCCAGGATGCTGCCCCCCCTTATGCTGGCCACACGGCAAT |
| | 81_Target6.3_9 | TTCTGAAGACACGCCAGAAATGCTATCTTTATTTTTATTAAAGGACAG |
| | | GGTCTCTGGCCTGGTGCGTTGGCTCA |
| 181 | 632327_266823 | TGTGAGTCACGTGCTTGGCCTAAACTTTTAAATGATGAGACTTAAGTC |
| | 82_Target6.4_1 | AAAGTTTATCCAAGGCTCTGCCCTGGCCCACCAGCCACCACAGTCTG |
| | | CCCTCTGCTTCTCACATGC |
| 182 | 632327_266823 | CAGAATTTCCCTGGAAGGAGATTCTCTAGAGCCCTTCCCACTGGAGT |
| | 83_Target6.5_1 | CAGGGGTGCTGGTGAAGAGCACTGGGGTCTGCAGGCTGGGTGGAC |
| | | CCCAAGCTTAGTTGGATCCTGGGCAAAT |
| 183 | 632327_266823 | CCAGGCTGCTAGAGGCTGGGGATCCCCAGCACACAGGCTCCAGGCT |
| | 83_Target6.5_2 | GGGCTCTCACTCTCATTCCACTCTCCTTACATGGGAGCCTTCCTGCC |
| | | AGAATTTCCCTGGAAGGAGATTCTCTA |
| 184 | 632327_266823 | CTCCTCTCCCCACAGGCCTGTGTGGCCCAGCCATGCGCTCTCCCAC |
| | 84_Target8.1_1 | TTGGGTCCCTGCCCTGGCCGCGGCCTGGGCTACCCACCCAGGTAGC |
| | | CAAAGGAATCTTTTTTTGTTTTGTTTTG |
| 185 | 632327_266823 | GTGTTAGTTATTACTCTCAACATCTCTTCAAACCACCTTTATGCTTGCC |
| | 84_Target8.1_2 | TCTCTCAGACTCCTCTTCCTCCTCTCCCCACAGGCCTGTGTGGCCCA |
| | | GCCATGCGCTCTCCCACTTGGGTC |
| 186 | 632327_266823 | GAGAGGCAAGCATAAAGGTGGTTTGAAGAGATGTTGAGAGTAATAAC |
| | 84_Target8.1_3 | TAACACTTGTTATCTCATTCAATCTTCACAAGATGCAATGAAGAAGTG |
| | | GGCTTTTTTTTTTTTTGAGACAGAG |
| 187 | 632327_266823 | AAATTCACCTATTATTCCAGCTTCTCTCTTCCAAATACGGCCTCTGTG |
| | 85_Target8.2_1 | CCCTGTTTCTTCCCCCAGGAGGAGATGAACGGCTGGCTGGAGGCTG |
| | | TAGCTTCCTCGGTGGCGGAACACGCA |
| 188 | 632327_266823 | GGGGTGGGAAGTCACTTCCTGCGCCCGCTGGCCCTGCGATCTCCGC |
| | 85_Target8.2_2 | CTTCCCTCTTAGGGTTGCCCTCATCTGTGGATGAAGTAGTGGGTAGT |
| | | GTCTGGCCCCAGCGGGCGATCTCTGCG |
| 189 | 632327_266823 | CCCACCATGTCACGCGTCCTCAGTGTTGGAACTAGGGGCTCCCGCC |
| | 85_Target8.2_3 | CCTGCGGAAGAAAAAGTGTCTTTGTGCCCACAGTGCGGAAAAGAGG |
| | | GGAGACGAGATGTGTCAGGTCCTGGGGG |
| 190 | 632327_266823 | GACCCCCAGCACCTGGGGTGGGGGTGGTGAGTGGGAAGGGGCCGA |
| | 85_Target8.2_4 | GATATGGCTATAGGGGCAGCAATCTCCCCACCACTTCTTGGGGTGCA |
| | | GGAGAAGTCCCCTCCTTTCCGGTGCCCA |
| 191 | 632327_266823 | TACTGCCCCCACCACCCCAGCTTCTTGGGGCAGGATCTGCAGATCG |
| | 85_Target8.2_5 | GGGCTGGGGGCTTGGCTCAACTGGCCCCCTGCCCACCCCCATCAAG |
| | | CTCAGTTATTGCATAAAAATAAGGGACC |
| 192 | 632327_266823 | GTGAGGTAAAGGCGGGGAGGGGGTGTCCCCTCCCTGCCCCCTAGC |
| | 85_Target8.2_6 | ACCCTGGATGGTCCAGGGGACCTCACCAGCGCCCCCGTGCCCATCC |
| | | CTAGGGCAGAGAGGGGGGCAGGAATTACT |
| 193 | 632327_266823 | TTTTGCAAAAATAAACTAAGTCAGGAATTAACCGCATGGCTCTGAGGC |
| | 85_Target8.2_7 | GGCGGAGTGCAGGGGGCTAGGGCCCCTTCACTCCCCCCTCCCAGA |
| | | CAGAGATCAAGGCAGCATTGGAAGTGA |
| 194 | 632327_266823 | CATGAAGTGGCATTTACAACTGGAGGGGGGAGGAGAGAGGAAAACA |
| | 85_Target8.2_8 | GTTTTGAGCTTTGACGCCCCTCCCATTAAAAGCCTTCGCGGATGCGG |
| | | GGCGGGGGAGGAGGAGATCGACGTTTT |
| 195 | 632327_266823 | CCCCAGCGGCTTCTCCAGGCGGCCATGGGGACTGCCCACAACCCAA |
| | 85_Target8.2_9 | TCCCCCGACTCCCCAGGGAGGTGCTCCAGCCAGAAGCATGCAGGGG |
| | | TGGGCTTCCCCTCGCCCCTCCCCTCATG |
| 196 | 632327_266823 | AATCCTCCCGGGCGCAGCCCAGAAAACCCAAACAAAGGACCATAGA |
| | 85_Target8.2_10 | CCGTCAAAGCCAAAGGGTTTATTATAGGTACATACAGTGTGCGCCCG |
| | | CCACACCGCCCCACACCCCCGGGCCCC |
| 197 | 632327_266823 | CCCCGAAATTAAGTCCACATAAAAGGGTACATCTGGATGTTCTCCGC |
| | 85_Target8.2_11 | AGCCACGGTACCCCCGTGCCCCTCTCCCCACCCCGCACCCACCCCT |
| | | ACAGCCACCACAGCCCTCCTTTCAATC |
| 198 | 632327_266823 | CGCCTTTCCTGAACTTGACGGGGCGGACCCGGAGTCTCCTTTGGGG |
| | 85_Target8.2_12 | AAGTTTATTACGAAGCATAACGAAATATTCTCCATCCCTCTAGGTTCT |
| | | CGTCAAGGGGCGTGTGTTTAGACCCC |
| 199 | 632327_266823 | GCACCCGGACCCCGCCTCCATAGGCTGCTCATTGGGCCAGAGACTC |
| | 85_Target8.2_13 | CAGGAGTCACTAGCGATTGGACAGAGCGCCTGCCCATCGCGGTTAA |
| | | CTTTTCCGCCTCCTTAAAGGGGTCCGCC |
| 200 | 632327_266823 | CATTCAGATGAATGACTTCCCCGGGAGGCCCCCGACTGGGGACCCC |
| | 85_Target8.2_14 | CTCGGCTGCAGTAGCCGCTGCTGCCATGGCCCCCGGGCGAGCCGG |
| | | CTGGCCCGCACCCACTTCCGGGTGTGCAC |
| 201 | 632327_266823 | GCGGAGCAACCCTTGATGGACAGGGATTCCCCCAGGAGAGGCGGAT |
| | 85_Target8.2_15 | ACTCCCACCCCGCTTCCCGCCCCCGAGGATGGGACCTCAGGAGTCT |
| | | CCCACACTCACCTCTTGCCTCCCACATT |
| 202 | 632327_266823 | AGAGATGAAGAGGAGCGGGTGAGGAATGCGTCCCCCTCCCGCAGGA |
| | 85_Target8.2_16 | TCCAGGGTTCCCTCCCCTTCCCCCTCCCCCGGCGTTTTTCCAGAATC |
| | | TCGCGCCTCCAAAGGGGGGGGGGGCGG |
| 203 | 632327_266823 | CTCCTACCCTCGCCCCAAACTCGCTCTCCCGATCACCTGGAGAAGAA |
| | 85_Target8.2_17 | GGAGTCTGGGATCCAGGTGAGAGTCTGGCGAGAGGTACTGAATCTG |
| | | GGCGGGGAAGGTAAACGGACGCAAGAG |
| 204 | 632327_266823 | GGAGGGATGGAGAGGGCGTGGGAGACCTAATATCCACTCCCCCGGC |
| | 85_Target8.2_18 | CCGTGACGCCTGCTCGGTGTCCCTTCCCTGCAGTCTTCTGAGCGGA |
| | | CGCATCTCGACGCTGAAAGATGAGACCG |
| 205 | 632327_266823 | GGGAAAGGGGGGCAGAGGATGGATGGGGTAAAAGGCAGGACTGAG |
| | 85_Target8.2_19 | TGGGGTCCTGCCCCTCTTCCTACCAACCCCTCCCCCCCATCCCATTT |
| | | CAGTTCTTGTCCGACTCACTGCTCCGGT |
| 206 | 632327_266823 | AGACAGTGAGGGACTCCCTCCCCTTTTTCCATGCCAACCTGGGATCC |
| | 85_Target8.2_20 | AACTCTTTGGTGCGCAGGAAGTTGAGGATGGGGGCGAAGACTGTAG |
| | | GGTCCCTGTCGATGAAGATCTGCGGGA |
| 207 | 632327_266823 | GTCTTCAGTCCCTGAGGAGTTCTTTCACCTCCTCTGAATGTCCACGT |
| | 85_Target8.2_21 | GAGCTCCATTCTATTAGAATTCTCATTCAGAACCTTGATCACAGGCCG |
| | | GGCGCGCCGGGATTGCT |
| 208 | 632327_266823 | ACACACACACACACACACACTCAACATTTCCTCCACCCATATCATCAC |
| | 86_Target8.3_1 | TCCTTAGCATCTTTATTCCATCAAAACTTTCTACCCCTTGACATTCTCT |
| | | GTGCAGTTTTGAAAATTACCCTC |
| 209 | 632327_266823 | GAGTGAGCCCATAGAACTGGGCTTCATGGAGGAGGCTGGAACCGTG |
| | 86_Target8.3_2 | GACACCCCTGGGTAGTAGAATTGACTAGAGGTCAGGGTCTAGGTGT |
| | | GGGGGTGAACAGAGAATGCTGAGAGGGT |
| 210 | 632327_266823 | CCACCTCTACCCCAGCCCTCTTTCAGGAGAGAAGGGGAAGAGGGGC |
| | 86_Target8.3_3 | AGGTAGCATCACAGAGTTGGTTTTCTATACAATGAGGATGATAGCTG |
| | | GGGGCTCCCACTTACCCAGAGGAGTGA |
| 211 | 632327_266823 | ACTTAGGGAGATGCTGAGGCCACCCCATTTCCCCCTCCCCATTTCTT |
| | 86_Target8.3_4 | TCTACACAGAGTACCAATACTCCTCCCAGTACGAATACAGACCCGGC |
| | | CAGGGCCATAACCAGTCCTCCCACCT |
| 212 | 632327_266823 | CTAAGTCCCAGCCTTTAAGTCCTCCTGTTGCAGTTCGTCGCCTGCAG |
| | 86_Target8.3_5 | CTTCGAGAGGAGTTGGATCGATCTTCTTGTGGAAACGTCCTCTTCAAT |
| | | GGTTACCTGCCGCCACCAGGTAGGC |
| 213 | 632327_266823 | GAACTACACCTCCCACTGTATCTCAGGACGCACAGCACAGGCACCC |
| | 86_Target8.3_6 | GAGAGCTGCAGCCTTATGGGAAATGTAGTCTTTGGCCCACCTCCGCG |
| | | CCCCTCCATTCCATTGGGAGTGCCTAC |
| 214 | 632327_266823 | TAGTTCCCTATCAGATGCTTGGGCTGATGCTTGGAAAGGAAGTTGGA |
| | 86_Target8.3_7 | CACAGCATTTCCCATGAGACAATGGGCCAGCTAACTCTTGAGGCTCA |
| | | GAAGGATGTCCTGGAGCCCCATGGGA |
| 215 | 632327_266823 | GGCTATCATGGGCATTACTGTCAATAACTTCCTATTAATATTTCTCCC |
| | 86_Target8.3_8 | CTCCATACGAAGAATCCCAGGTCTTCCAAAGCCCCCCCTACAAAGAA |
| | | ACCAAACCCGGACTACAACTCCCAT |
| 216 | 632327_266823 | ATAGCCAGCCCACTGGCCATGGAAGGTATGCCCCAGTGGTTATTGGA |
| | 86_Target8.3_9 | ACTAGGCTTTTCTGATTGGTAGAAGTAACAGAGTAGGGAAATTTCATC |
| | | TACAGCTTTATTTCCCTAACTGCAG |
| 217 | 632327_266823 | CTGCAGTCAGCACCTGCTACCTTCATGAAAGTTGCCAGATATAAAGAT |
| | 86_Target8.3_1 | CTGTAGTAGTACTTTTCCAACTTAGTTTTATCCTGTTTTCCGAAAAACA |
| | 0 | ATCATTTATTTATTTATTTATTT |
| 218 | 632327_266823 | CATTATATACAACCCAAGGCTTGCCACAAACTGAAATTTCTTATGCAT |
| | 87_Target5.1_1 | TGACATTCAGCATTTGATGTAAAATGACAATCTTAGAGTCGCTGCCAC |
| | | AAACCCTGGTAAAACTGCCCCTGC |
| 219 | 632327_266823 | CTTCTCCAGTTCACTAAGTTCAGCTACTCCTTCAGGGCTTGTGCTCCT |
| | 87_Target5.1_2 | TTAAAACTTTTCAGAAGCCCCTTGAAGTACCAACATGTTGAAACTCAA |
| | | ACTCAACTGCCTGCAGCTTCAGAT |
| 220 | 632327_266823 | CAGCTCTGGCCACTGAGAATTCTTCTCCAGGCCCCAGTGTCAAAGGC |
| | 87_Target5.1_3 | ACTTTTGACCATCATGTGTTTGTCTGACTACCAAGTTGGACTGCCAAC |
| | | CTTCACTAAAAACTCCGGCACCTTT |
| 221 | 632327_266823 | ATCTACCTCCTGTCAGATCACCGCCTGTCAGAGAGGGAGGCCTAAAA |
| | 87_Target5.1_4 | AGGCTTCAGGATATCAACAGCTTTGAGCTGCCTTAGAGGAACTTCTTT |
| | | TCTATTTTGTAGTACAGGAGCCTCA |
| 222 | 632327_266823 | TGCACTGCATCATCCAAACAAGACTCTCCACAAAACATTACTAATGGA |
| | 87_Target5.1_5 | TTCTAATTGAACAGAGTTTGGTTACAAGAGACAGTAAACACAAATGGG |
| | | CACCTGTTTTAAATGGCAAACTTA |
| 223 | 632327_266823 | CACAATAAATTCAATTGATCTTAAAAACTAAACATTCCTCTAAGGTTAC |
| | 87_Target5.1_6 | TGACTATAAATATCTAGAAATTACTACTCCTATAGTCTTGCTATTTCCA |
| | | GACAAGTGAAACTGGAAGGGAG |
| 224 | 632327_266823 | AGAGGAGAAATACCCAGTTATAATGACAAGACAACCTTATCTTTTCTC |
| | 87_Target5.1_7 | AAAAGCCTCCCCAAACACACAACCATTTTCCACAAAACAGTTACATCA |
| | | AAAAAGATGTAGCAAGACCTAGAC |
| 225 | 632327_266823 | ATTAAGCATGCTAGAAAGTAGGGACAGTTTTACCAGTGTTTGTGGCA |
| | 87_Target5.1_8 | GCAACTCTGAGATAGTCATTTTACATCAAATGCTAAATGTCAATGCAT |
| | | AAGAAATTTCAGTTTGTGGTAAGCC |
| 226 | 632327_266823 | CTGGGTTATATATAATGGCAACAAGAGAAAACTATCACATAATCTGAA |
| | 87_Target5.1_9 | TTACGTTCATTTTTCTGCATACACCATGCATGCTGGTACACTTTCAGTT |
| | | AGCCTAAACTTCACCCAAAAGTA |
| 227 | 632327_266823 | GAGGTGGCTATGAAAACTCTACCTGCATGGTATAAATAGATACCTAAC |
| | 87_Target5.1_10 | CCTACATGCATGGGTCACTTGTATCTACCTCCTAAATACCAAAAACTT |
| | | AGAAGCCCAGATTTCTTTTCAGTC |
| 228 | 632327_266823 | AAAAACAAAACAAAACAAAAAATTACCCTTCCCCACAATTATAAGCATT |
| | 87_Target5.1_11 | TTGGCATATTTCCTTCCTGTCTCTGTTGTTCCTATTTTGGCAGTATTTT |
| | | TATTGTTATCGCAGGCAGCCT |
| 229 | 632327_266823 | GCCGCACACCTTATACCTCTCCCCTTTAAAACAGACATATTAAAGACT |
| | 88_Target5.2_1 | TCTGTTTTCTGAATTTACTGTAAACAGAATACTTAAAAAAAAATTACCC |
| | | AGACAGGCGTGGTGGCTCACTGT |
| 230 | 632327_266823 | TGAACAAACTGCATTAGACTAAAATCTTTCACGAGTCCAAATTCTGCG |
| | 88_Target5.2_2 | ACTAGAAAAATAAACTTGTTAGCCGCACACCTTATACCTCTCCCCTTT |
| | | AAAACAGACATATTAAAGACTTCT |
| 231 | 632327_266823 | ACTTACTTTCTTGTTCTGACTCAACTCGGAAAACACCTCCCTAGTCTG |
| | 88_Target5.2_3 | AGGCCCCCCTCCTTATATTTGTGAACAAACTGCATTAGACTAAAATCT |
| | | TTCACGAGTCCAAATTCTGCGACT |
| 232 | 632327_266823 | TTTCCACTATATCACACTGCACTGTCTGGCTCCAATCTGATGGCGAG |
| | 89_Target5.3_1 | CAATGGTTCCTTAAAAAGCTCCATCTTCTCAAGCCTACCCAGCACAGA |
| | | CCTACAACTCAAACTTTAAGTTTTA |
| 233 | 632327_266823 | AGCCAGCCTAACCTTCCACGTCCACTTGAAAAAAGAAAAAAAAGAAA |
| | 89_Target5.3_2 | GAAACAATCAACCGGAAGCCTTTGATTTTTTTGCTAGCAAGAAGCCAA |
| | | TAATTGTTTTCTTTGGGACAATCAC |
| 234 | 632327_266823 | CAGGTTAAGTTGTGAAATGGGTGGCAGAAACTGCAAGATTTAACCAG |
| | 89_Target5.3_3 | TTTCTCTCAACGCTAATCAAAGGTCAAGAAAATAAAAGACAGGGCAGT |
| | | GAGAGTCGCGGGACTTTAGACACTC |
| 235 | 632327_266823 | AGTGTGGAAACCCCTTCCCCACTCAGTCCAATTTCAAGGTTATGGAG |
| | 89_Target5.3_4 | CATAATTAAGAGTGACCATTCTAAAAGCGTTCTCTAGTGCCCTGGCTA |
| | | AGACTTTTAAGCTTCTTGGTGCAGG |
| 236 | 632327_266823 | AGAAGCAAAGATGATGTCTGTCACCTGGCAGCGTAGACGGAGAGTG |
| | 89_Target5.3_5 | GCGGTAGATGACTTGAATGGACACGTGGATTCGAGTCTGGGGACAG |
| | | GCGTTCCCCCAGTCGGTGCGTTGTGAGA |
| 237 | 632327_266823 | TCAATTTCAAAAAATGCCAGCACTCCTTAGATCAATGACTCCACGGCC |
| | 89_Target5.3_6 | AAGTGCAAGGGCCACGGGCACTCCTCTGGAGTCACCGAGAGAATGT |
| | | CTGCCTCTGGCAGCGGCCTCTGAGCT |
| 238 | 632327_266823 | GGCCCCCGCCTGCCCAGAGGCCGGGCTCGCGGGCGTTCGGACCGC |
| | 89_Target5.3_7 | GCGTGGGGCGAGACTGCACTCGCGCCCGCTGAGATGCCGGCCCCA |
| | | CGCAGCGCGGCGGCACGAGGCCCACAGGCA |
| 239 | 632327_266823 | CGCGGACGAGGGCAACGTCAGCGGCCCAGCTGGGCCACCTCCTCC |
| | 89_Target5.3_8 | CAGCCGAAGCGCGGCCTCCGGCTGCTTCGCCGGGCCGGTCCCCGC |
| | | CGCCGCACACACAAGGGTCGGTCCCGCGCG |
| 240 | 632327_266823 | CTGCCCGCGGCCCCGCTTCCCTCGGCAGCCGGAGGCCTCCGGGGT |
| | 89_Target5.3_9 | GCCCGTGCTGCCGCAGCTTCCTGTGTCTGAGTCGCGGGCGGCGCG |
| | | GGCGCGGGGCTGGTGCGCGGCCGGGGATGC |
| 241 | 632327_266823 | GGGCCCCGGCCGAGGCGCCGGCTGACGTTGCCCTTCAGGGCCGAG |
| | 89_Target5.3_10 | CCCGGAGCAGCGGCCGCCGCGGAGGCGCGTGAGCCACCCCAGGCT |
| | | CGTGGGGCCGCGCCGCCTTTCCGGCCTCC |
| 242 | 632327_266823 | GCCGCGGGAGCAGGCGCGGCTCCGCACGGCGGGCGCACTCACCTC |
| | 90_Target5.4_1 | CTCCGTGGACCGGCCGCTCCGCCGCCGGCTGTCACTGCAGCCCACT |
| | | CCCTGCCCCGCGCCGGCTCTGGATGCTAT |
| 243 | 632327_266823 | TCTGGATGCTATTTAAGGCGCCGCTGGCGCCGCCGCCTCGGCCCGC |
| | 90_Target5.4_2 | TGGTTGCGCCCCCGCCACCGCCGCGGCTGAGCCAGGATGCTGCTG |
| | | GTGCGCTCGCCGCGCCGGCCCCGCCCTCC |
| 244 | 632327_266823 | CGCCGAGCGCCCGGCCGCCCTCCTGTCTGGGCGCGCGTGGCTGGC |
| | 91_Target5.5_1 | GGGTGGCCGGCGGTCGAGAGGCTGCGGCGGGCGAACGCCGCTGG |
| | | AGTCGCCCAGACTGCCTCGGATTTCATAATG |
| 245 | 632327_266823 | CAGGATCCGGAGGGCGCCGCCCGCCCCTCGGGCATCGCGGGGCCC |
| | 91_Target5.5_2 | AGGGTCCGCGGGGCCTCTGAGCTGAGGCGGTCCAATGTACCCTTTG |
| | | TCCTTCCTCAGATTCATTATGAAATCCGA |
| 246 | 632327_266823 | GCCCTCCGGATCCTGCACCCTCAGCCCGTGCCCCGCTGGCCTCCTG |
| | 91_Target5.5_3 | GCAGGCCGGGCAGAATTCTGCTGCGAGGGCCCTGGAGGCAAGGGC |
| | | CTTCCTCTCCGTTTTTCCTTGACACTGAC |
| 247 | 632327_266823 | TTCCTTGACACTGACGGGCGCGGCCCCACAGGGTTTCCCCCACGCT |
| | 91_Target5.5_4 | CCCTGGCCGGAAGACCTTTTCTGTCTGGTGTCTGCAGAGCCTGACGT |
| | | GGGCAGCGCCCGCAGCTCGCGCCGGTG |
| 248 | 632327_266823 | TGGGCATGGCAGGGACGCGGAAGCCCTCAGAACCGCGGAAGCCCT |
| | 91_Target5.5_5 | CAGAACCGCACAGCAGGTGGCCGCCGTGGTGCAGCGCGCAGTCGG |
| | | CCCTCAGCTCCCCTGCACCGGCGCGAGCTG |
| 249 | 632327_266823 | GTTGTTAGAGGTTGAGGTTCCTGGTAGGTCCCAGGTGCTAAAAGGAA |
| | 91_Target5.5_6 | GAGCAGCCTTTGGGATGTGAGCCCGAAAACCATCGTGTTGGCTTCTG |
| | | AAGCCCCAAGATGGGCATGGCAGGGA |
| 250 | 632327_266823 | TCAACCTCTAACAACATTGGTCCCCGTTACCGTGGAGTTTGCGCCCA |
| | 91_Target5.5_7 | GCGTTCGCTGGAGGGCCGTTTGAAGAATGATTGTTGTCGTAAAGGCC |
| | | TTTGATAATAATAGATACTGTTTACC |
| 251 | 632327_266823 | GTCAGTTTCCCCTGCCTGCAGCCCAGGCATAGCCCTGCGTTGGTTGT |
| | 92_Target5.6_1 | GCCCACACTCTGGGGGCAGCCTGAGTGCCAAGTGTGGTACTGCTCT |
| | | CACTAGCATTTAACCTCTGTGCCCCAG |
| 252 | 632327_266823 | TGCGCCCAGCACACTGTGGGAGACACCCAGGGAAAGTCCTTCAGTT |
| | 92_Target5.6_2 | ATTTGTGGGAGTGAACTGCAGTGTGGCTGAGGCCTGGAGCGAACCA |
| | | CGGTCAGTTTCCCCTGCCTGCAGCCCAG |
| 253 | 632327_266823 | CCATTTTTTCTAGATCTGGAAGATGGAGATCTAAAAGGACTGAGCTGC |
| | 92_Target5.6_3 | TAGAGAGGTTGCTGGTGTGTGCGTTGTCCTCACCCCACTGCACCACT |
| | | GCGCCCAGCACACTGTGGGAGACAC |
| 254 | 632327_266823 | CCATCTTCCAGATCTAGAAAAAATGGACTCCACGTTCGTTTTCACTGG |
| | 92_Target5.6_4 | TTCCCGCTTGGCTAATCCCTGGTTTTTCTGTCCAGATCTCGCCTCCCC |
| | | ATCCGTGATTTTGTCATCTTTGCC |
| 255 | 632327_266823 | CCATCCGTGATTTTGTCATCTTTGCCTCTCCTTTTTCCTCTTCTCCAAC |
| | 92_Target5.6_5 | CGGTCCCTCAGCCCCAGAACCTCTGCAGGCACCTCCCAAGTTCTGT |
| | | GGTCTATTGTTTTCAAGGGCTGCTG |
| 256 | 632327_266823 | TGACAATTCAACCCGTAACATGTGGATGGTCGTCACTGGCTGTCTCA |
| | 93_Target5.7_1 | CCTTACGTCTTCTCCTCAAGACACTTTTCTTTTAAACTGGGTGCCTCT |
| | | CCCTGGGCTCTAGCCCTTAATGAGG |
| 257 | 632327_266823 | AAGACACTTTTCTTTTAAACTGGGTGCCTCTCCCTGGGCTCTAGCCCT |
| | 93_Target5.7_2 | TAATGAGGTGATTTCCAACTCAAGGCTGGGAAAGGCACACAACCCAG |
| | | TATCAGACCTGAGTTCACACTCCTT |
| 258 | 632327_266823 | CCCATACATTGTAAAACTTTTTGGAGTGATTAGATAAAAACTTTTTTGT |
| | 94_Target5.8_1 | TTTTTTTTAAGTGTGTGTGCTGAAAGAGACCTAAGAGATTATTTAGCC |
| | | AAGCCCCCTTATAGCCTGAGGAG |
| 259 | 632327_266823 | TCTTAACCACTATGCAATACTGCCTCCCATCAAAGTCTCTATCTCAAG |
| | 94_Target5.8_2 | TCCACAAGACAACCCCATACATTGTAAAACTTTTTGGAGTGATTAGAT |
| | | AAAAACTTTTTTGTTTTTTTTTAA |
| 260 | 632327_266823 | GACAGGTGTCATAGGATTGACAGCCACGCCCTTGGCGTTCTCATTAA |
| | 95_Target5.9_1 | ATGAAGATTCCAGACTCTATAGTTGGGAGTTTGTGACTTAGGTAAGAA |
| | | ACCAGTCTCTTTTTTTGAGACGGAG |
| 261 | 632327_266823 | TGTCATAGGATTGACAGCCACGCCCTTGGCGTTCTCATTAAATGAAG |
| | 95_Target5.9_2 | ATTCCAGACTCTATAGTTGGGAGTTTGTGACTTAGGTAAGAAACCAGT |
| | | CTCTTTTTTTGAGACGGAGTCTCAC |
| 262 | patch | CTCTGTCGCCCAGGCTGGAGTGCAGTGGCACAATCTCTGCTCACTG |
| | | CAAGCTCCGCCTCCCGGGTTCACGCCATTCTCCTGCCTCAGCCTCCC |
| | | GAGTAGCTGGGACTACAGGCGCCCGCT |
| 263 | patch | GCCCGCCTCGGCCTCCCAAAGTGCTGGGATTACAGGCGTGAGCCAC |
| | | CGCGCCCGGCCCACAGCTCTAGTTCTTTAATACACATCAGGTCGGTC |
| | | ATGGTGGCTCATGCCTGTGATCCCAGC |
| 264 | patch | TTGCTATGTTGCCCTGGCTGGTCTTGAACTGCCAGCCTTCAGGGACG |
| | | CTCCTGCCTTGACCTCCCCAAATGCTGGGATCACAGGCATGAGCCAC |
| | | CATGACCGACCTGATGTGTATTAAAG |
| 265 | patch | GAGCGTCCCTGAAGGCTGGCAGTTCAAGACCAGCCAGGGCAACATA |
| | | GCAAGACCCCATTTCTACAAAAAATTAAACAGCCAACCAAAAAAAAAA |
| | | AAAAAAAGAAAGAAAAACTTTAGCTG |
| 266 | patch | CCTGGGCTCAAGCAATTCTCCTGCCTCAACCCCCCAAGTAGCTGGGA |
| | | CCACAGGCATGCTCCACCATGTCCAGCTAAAGTTTTTCTTTCTTTTTTT |
| | | TTTTTTTTTTGGTTGGCTGTTTAA |
| 267 | patch | GACAGGGTCTGGCTGTGTCACCCAGGCTGGAGTGCAGTGGTGTAAT |
| | | CATGGCTCACTGCAGCCTCGACCTCCTGGGCTCAAGCAATTCTCCTG |
| | | CCTCAACCCCCCAAGTAGCTGGGACCA |
| 268 | patch | CATGATTACACCACTGCACTCCAGCCTGGGTGACACAGCCAGACCCT |
| | | GTCTCAAAAAATATATATGTATATTTTAAAATATTATATATTATATAATG |
| | | TAAATAATATACATGCTACATAT |
| 269 | patch | TTTTAAAATATTATATATTATATAATGTAAATAATATAGATGGTAGATAT |
| | | TTTATGTATACATCTATGTTATATACATTAATATAGCCAGGTGCAGTGG |
| | | CTCACACCTGTGTTCCCAGCA |
| 270 | patch | ATATACATTAATATAGCCAGGTGCAGTGGCTCACACCTGTGTTCCCA |
| | | GCACTTTGGCGGGATTACGGGCGGGTCACCTGAGGTCAGGAGTTCG |
| | | AGACCAGCCTGGCCAATATAGTGAAAC |
| 271 | patch | GGTCACCTGAGGTCAGGAGTTCGAGACCAGCCTGGCCAATATAGTG |
| | | AAACCCCGTCTTTACTAAAAATACAAAAAAAAAAAAAAATTAGCCAGG |
| | | CGTGGTGGTGGGCGTCTATAATCCCA |
| 272 | patch | AAAAAAAAAAAAAAATTAGCCAGGCGTGGTGGTGGGCGTCTATAATC |
| | | CCAGCTATACAGGAGGCTGAGGCAGGAGAATTGTTTGAACCCGGGA |
| | | GGCGGAGATTGCAGTGAGCTGAGATCG |
| 273 | patch | TTCCCCTTTGGATTTTTTTTTTTTTTTGAGACAGAGTCTCGCTCTGTCA |
| | | CCAGGCTGGAGAGCAGTGGTGCGATCTCAGCTCACTGCAATCTCCG |
| | | CCTCCCGGGTTCAAACAATTCTCCT |
| 274 | patch | GTGCTGATGGTAATAACCTTGACCACGTGGTCAGGGCATCTGCCGGT |
| | | TCCTGCACTGTCCAGTTACTGTTTTGGCCTTTGGATTTTTTTTTTTTTTT |
| | | GAGACAGAGTCTCGCTCTGTCAC |
| 275 | patch | GGGAGTCCCGGGCGTGGCGTGTGTCTGTCTCAGGCCCCCCACGTC |
| | | GAAGCCCATGGCTTCGGCCTGTCTCGTGCTGATGGTAATAACCTTGA |
| | | CCACGTGGTCAGGGCATCTGCCGGTTCC |
| 276 | patch | ACTCAATGGAGGGAGTCCCGGGCGTGGCGTGTGTCTGTCTCAGGCC |
| | | CCCCACGTCGAAGCCCATGGCTTCGGCCTGTCTCGTGCTGATGGTA |
| | | ATAACCTTGACCACGTGGTCAGGGCATC |
| 277 | patch | CCTCTAACCCTGGGGTGATTCTGCCCCCAGGGGTCCCTGTGTGAGG |
| | | CCTGGAGACATCCGTGGTTGTCACGACTTGGAGGGAGCTCCTGGCA |
| | | GGGCCCTGCAGTGCCCAGGATGGCCCCG |
| 278 | patch | CTCAGAGTCTCTTGGGGTCTGTGGAATCCTCTAACCCTGGGGTGATT |
| | | CTGCCCCCAGGGGTCCCTGTGTGAGGCCTGGAGACATCCGTGGTTG |
| | | TCACGACTTGGAGGGAGCTCCTGGCAG |
| 279 | patch | AGGGAGCCAAGATCGTGCCACTGCACTCCAGCCTGGGCAACAGAGC |
| | | AAGACTCCATCTCAAAACATAAATAAATAAAAATAAAAAATAAAAAATC |
| | | CAGTCTGCCCCCTGCTCACACCGGA |
| 280 | patch | TCTTGCTCTGTTGCCCAGGCTGGAGTGCAGTGGCACGATCTTGGCTC |
| | | CCTGCAACCTTCGCCTCCCAGGCTCAAGTGATTCTCCTGCCTCAGCC |
| | | TCCTGAGTAGCTGGGACTACAGGCGC |
| 281 | patch | ACCAGCCTGGCCAACATAGTGAAACCCCATCTCTACTAAAAATACAAA |
| | | AATTAGCTGCGCGTGTTGGCGGGCGCCTGTAGTCCCAGCTACTCAG |
| | | GAGGCTGAGGCAGGAGAATCACTTGA |
| 282 | patch | GCCTCACACCTGTAATCCCAGCACTTTGGGAGGCCGAGGCGGGTGG |
| | | ATCGCCTGAGGTCAGGAGTTCAAGACCAGCCTGGCCAACATAGTGAA |
| | | ACCCCATCTCTACTAAAAATACAAAAA |
| 283 | patch | CGATCCACCCGCCTCGGCCTCCCAAAGTGCTGGGATTACAGGTGTG |
| | | AGGCACCGCACCTGGCCATTTTTTTCCCCAAATAATGAACACTGGTCT |
| | | CTCCCCTGGGCCCTGTGGACATCGGG |
| 284 | patch | GTGGAGGCCAAGGATGATGCCTAGCACCCTGCAGTGCCCAGGACGG |
| | | GCCTGCCCCAGAGAAGGATCCAGCCCCGATGTCCACAGGGCCCAGG |
| | | GGAGAGACCAGTGTTCATTATTTGGGGA |
| 285 | patch | AGGCCCGTCCTGGGCACTGCAGGGTGCTAGGCATCATCCTTGGCCT |
| | | CCACCCACTCCATGCCAGGAGCTCCCCTTTCGTGACAGCCACAGGT |
| | | GTCCTCAGACCTCGCCCAGGGTCCCCTG |
| 286 | patch | TGGGGGTTTTCTTCCCCCTTTAAGGCCAGGATTCTCAATCAAGAGGT |
| | | GATTCTGCCCCCAGGGGACCCTGGGCGAGGTCTGAGGACACCTGTG |
| | | GCTGTCACGAAAGGGGAGCTCCTGGCA |
| 287 | patch | GAATTCGCCTGTTACTAGCTAATTCCCAAAACAGCTCCTCAGGAGGG |
| | | AAGGGCTGGGTTTTGCCCGTTTTACAGATAGGCAACTGAGTCAAATG |
| | | GCCAGGGCTCCCCCTTGCCCTTGTCC |
| 288 | patch | CTTCCCTCCTGAGGAGCTGTTTTGGGAATTAGCTAGTAACAGGCGAA |
| | | TTCCTGGACAGCACCCAGCCTTCAGCAAATGCTCAGTGAATATGCAT |
| | | GAATGAATGAATGAGTGAATGAATGA |
| 289 | patch | CCCCTCAGGTCCCTATCCTGAGGGTGATTTTCTTTTTCTTTTTCTTTTT |
| | | TTTTTTTTTGAGACGGAGTCTCGCCCTGTCGCCCAGGCTGGAGTGCA |
| | | GTGGCGCGATCTCGGCTCACTGCA |
| 290 | patch | CGCCCTGTCGCCCAGGCTGGAGTGCAGTGGCGCGATCTCGGCTCAC |
| | | TGCAAGCTCAGCCTCCCGGGTTCACGCCTTTCTCCTGCCTCAGCCTC |
| | | CCGAGTAGCTGGGACTACAGGCGCCCA |
| 291 | patch | AGACCATCCTGGCTAACACCTTGAAACCCCGTCTCTACAAAAATTACA |
| | | AAAAATTATCCAGGCATGGTGGTGGGCGCCTGTAGTCCCAGCTACTC |
| | | GGGAGGCTGAGGCAGGAGAAAGGCG |
| 292 | patch | CGGTGGCTCACGCCTGTAATCCCAGCACTTTGGGAGGCTGAGGCAG |
| | | GCGGATCACGAAGTCAGGAGATCGAGACCATCCTGGCTAACACCTT |
| | | GAAACCCCGTCTCTACAAAAATTACAAA |
| 293 | patch | CCGCCTGCCTCAGCCTCCCAAAGTGCTGGGATTACAGGCGTGAGCC |
| | | ACCGCGCCTGGCCCTTTTTTTTTCTTTTTTGAAACGGAATCTTGCTCT |
| | | GTCGCCCAGGCTGGAGTGCAGTGGTG |
| 294 | patch | GCTACTCAAGAGGCGGAGGCAGGAGAATCATTTGACCCCAGGAGGC |
| | | TGAGGCTGCAGTAAACTGAGATTGCACCACTGCACTCCAGCCTGGG |
| | | CGACAGAGCAAGATTCCGTTTCAAAAAA |
| 295 | patch | GTGGTGAAACCCCATCTCTACTAAAAATACAAAAACTAGCCAGGTGT |
| | | GGTGGTGTGTGCTTGTAATCCCAGCTACTCAAGAGGCGGAGGCAGG |
| | | AGAATCATTTGACCCCAGGAGGCTGAG |
| 296 | patch | TTTGCAGATGAACAAACTGAGGCATTGAAAATCCCCCTCAGGTCAGG |
| | | AGTTCGAGACCAGCCTGACCAACGTGGTGAAACCCCATCTCTACTAA |
| | | AAATACAAAAACTAGCCAGGTGTGGT |
| 297 | patch | AACAAAACAAAACAAAATAAAAAAAAAGAAGCAAAAAAAAAAAAAAAA |
| | | ATCAGCCAACAAAGAGACCCATTTTGCAGATGAACAAACTGAGGCAT |
| | | TGAAAATCCCCCTCAGGTCAGGAGT |
| 298 | patch | AAAAAAAAAAAAAAAAAAAAAAAATCCTGGGCGCAGTGGCTCATGCCT |
| | | GAGTGAGACCCCATCTCAAAAAAACAAAACAAAACAAAATAAAAAAAA |
| | | AGAAGCAAAAAAAAAAAAAAAAAT |
| 299 | patch | TCAGGCATGAGCCACTGCGCCCAGGATTTTTTTTTTTTTTTTTTTTTTT |
| | | TGAGACAGAATCTCACTCTGTTGCCCAGGCTGGAGTGCAGTGGTGTG |
| | | ATCTCGGCTCACTGCAACCTGTGC |
| 300 | patch | TGCCCAGGCTGGAGTGCAGTGGTGTGATCTCGGCTCACTGCAACCT |
| | | GTGCCTCCTGGGTTCAAGCGATTCTCCTGCCTCAGCCTCCCAGGCA |
| | | GCTGAGATTACAGGTGCGAACCACCACA |
| 301 | patch | TCCTGCCTCAGCCTCCCAGGCAGCTGAGATTACAGGTGCGAACCAC |
| | | CACACCTGGCTAATTTTTTTTTTTTTTTTTTTTGAGACGGAGTGTCATT |
| | | CTGTTGCCAGGCTGGAGTGCAGTGG |
| 302 | patch | TACTCCGGAGGTTGACGCAGGAGAATCGCTTGAACCCAGGAAGCGG |
| | | AGGTTGTGGTGAGCCAAGATCGCCCCACTGCACTCCAGCCTGGCAA |
| | | CAGAATGACACTCCGTCTCAAAAAAAAA |
| 303 | patch | ACCTCCGCTTCCTGGGTTCAAGCGATTCTCCTGCGTCAACCTCCGGA |
| | | GTAGCTGGGACTACAGGCACGTGCCACCACGCCCAGCTAATGTTTGT |
| | | ATTTTTAGTAGAGACAGGGTTTCACC |
| 304 | patch | CCACCACGCCCAGCTAATGTTTGTATTTTTAGTAGAGACAGGGTTTCA |
| | | CCATGTTGGCCAGGATGGTCTCCATCTCTTGACCTCAGGTGATCCAC |
| | | CCACCTCGGCCTCCCAAAGTATTGG |
| 305 | patch | CATCTCTTGACCTCAGGTGATCCACCCACCTCGGCCTCCCAAAGTAT |
| | | TGGGATTACAGGCGTGGGCCAACCGCACCCGACCAATTTTTCTGCTT |
| | | CTTTAAAAACATTTTTTTAAATTTTG |
| 306 | patch | CGCACCCGACCAATTTTTGTGCTTCTTTAAAAACATTTTTTTAAATTTT |
| | | GTTTTAGAGACAGGGTCTTGCTCTGTTGCCCAGGCTGGAGTGCAGTG |
| | | GTGTGATCTCAGTTCACTGCAGCC |
| 307 | patch | TCTGTTGCCCAGGCTGGAGTGCAGTGGTGTGATCTCAGTTCACTGCA |
| | | GCCTTGACCTCCTGGGCTCAAGCGATCCTCCCTCCTCAGCCTCCCAA |
| | | GTAGCTGGGACGACAGGTGCACACCA |
| 308 | patch | TCAAGACCAGCCTGGGCAAAATTGTGAGGACCCCATCTCTACAAAAA |
| | | ATTTTAAAATTAGCCAGGCATGGTGGTGTGCACCTGTCGTCCCAGCT |
| | | ACTTGGGAGGCTGAGGAGGGAGGATC |
| 309 | patch | GCAGTGGCTCACGCCTGTAATCCCAACACTTTGGGAGGCCGAGGCA |
| | | GGAGGCTCCCTTGAGCTCAGGAGTTCAAGACCAGCCTGGGCAAAAT |
| | | TGTGAGGACCCCATCTCTACAAAAAATT |
| 310 | patch | AGGCTGGTCTTGAACTCCTGAGCTCAAGGGAGCCTCCTGCCTCGGC |
| | | CTCCCAAAGTGTTGGGATTACAGGCGTGAGCCACTGCACCCAGCCA |
| | | CCTGGTCTGCGTCTTAAAAGCCTTCCTG |
| 311 | patch | GAGGTGGAATCACTTGAGCCTGCAGTGAGCTGAGATTGTGTCACTGC |
| | | ATTACAGCCTGGGCAACAGAGGGAGACCCTCTCAAAAAAATAAAAGG |
| | | CACGCACGCCCCCAAACCTGAAATGA |
| 312 | patch | AATGCAGTGACACAATCTCAGCTCACTGCAGGCTCAAGTGATTCCAC |
| | | CTCAGCCTCCCGAGTTGCCTGGATTATAGGCACACACCATCATGCCT |
| | | GGCTAATTTTTGGGTTTTTGTTTTTT |
| 313 | patch | TGAGCAACATATTGAGACCTCATCTCTACCAAAAACAACAAAAACAAC |
| | | AAAAAAAACCCCTAAAAAACACAAAAAACAAAAACCCAAAAATTAGCC |
| | | AGGCATGATGGTGTGTGCCTATAA |
| 314 | patch | TTGTTGTTTTTGTTGTTTTTGGTAGAGATGAGGTCTCAATATGTTGCTC |
| | | AGGCTGTTCTCCAACTTCTGGGCTCAAGCCATCCTCCTGCCTCCCAA |
| | | AGTGCTGGGATTACAGATATGAAC |
| 315 | patch | CAACCCCAAGAATAGAAAAGGCACTCACTGGCCAGTCGAAGTGGTTC |
| | | ATATCTGTAATCCCAGCACTTTGGGAGGCAGGAGGATGGCTTGAGCC |
| | | CAGAAGTTGGAGAACAGCCTGAGCAA |
| 316 | patch | GCTCTGAAGGAAAAGCAGGCCAAGTGGGTTTCTTCCTTTTTTTTTTTT |
| | | TTTGAGATAGAATCTCTTGCTCTGTCACCCGGGCTGGAGTGCAATGG |
| | | CACAATCTTGGCTCACTGCAATCTC |
| 317 | patch | TGTCACCCGGGCTGGAGTGCAATGGCACAATCTTGGCTCACTGCAAT |
| | | CTCCGCCTCCCGAGTTCAAGCAATTCTCCTGCCTCAGCCTCCTGAGT |
| | | AGCTGGGATTACAGGCGCGTGACACC |
| 318 | patch | TTCTCCTGCCTCAGCCTCCTGAGTAGCTGGGATTACAGGCGCGTGAC |
| | | ACCATGCCTAGCTAATTTGTATATTTTTAGTAGAGACAGGGTTTCA |
| | | CCATGTTGGTAAGGCTGGTCTTGAA |
| 319 | patch | TATTTTTAGTAGAGACAGGGTTTCACCATGTTGGTAAGGCTGGTCTTG |
| | | AACTCCTGACCTCGTGATCCGCTGGCCTCGGCCTCCTAAAGTGCTGG |
| | | GATTACAGGTGGGAGCCACCGCACC |
| 320 | patch | TGGCCTCGGCCTCCTAAAGTGCTGGGATTACAGGTGGGAGCCACCG |
| | | CACCAGGCCTCTTTTTTTTTTTGAGACGGAGTCTTGCTCTGTCGCCCA |
| | | GGCTGGAGTGCAGTGGCGCGATCTCG |
| 321 | patch | GACGGAGTCTTGCTCTGTCGCCCAGGCTGGAGTGCAGTGGCGCGAT |
| | | CTCGGCTCACTGCAAGCTCTGCCTCCTGGGTTCACGCCATTCTCCTG |
| | | CCTCAGTCTCCCGAGTAGCTGGGACTA |
| 322 | patch | CCTGGGTTCACGCCATTCTCCTGCCTCAGTCTCCCGAGTAGCTGGGA |
| | | CTACAGGCACCTGCCACTACGCCTGGCTAATTTTTTGTATTTTTAGTA |
| | | GAGACACAGTTTCACCGTTTCTACC |
| 323 | patch | AAGGAATGAGGAGCCCCAGGAGGTCTTGGTAAGAGATAAAAAAACAG |
| | | GAGGGGGCCCTCTCTGGCTCTATGGTAGAAACGGTGAAACTGTGTCT |
| | | CTACTAAAAATACAAAAAATTAGCCA |
| 324 | patch | ACCTGTAATTCCAGCACTTTGGGAGGCCGAGGCGGGCGGATCACAA |
| | | GGTCAGGAGATCGAGACCATCCTGAAGGAATGAGGAGCCCCAGGAG |
| | | GTCTTGGTAAGAGATAAAAAAACAGGAG |
| 325 | patch | GACCTTGTGATCCGCCCGCCTCGGCCTCCCAAAGTGCTGGAATTACA |
| | | GGTATGAGCCACCGCACCCGGCCTTTTTTTTTTTTTTTTGAGAAGCAG |
| | | TCTCTGTTGCCCAGACTGGAGTGCA |
| 326 | patch | TTTTTTTTTTTTTTTTGAGAAGCAGTCTCTGTTGCCCAGACTGGAGTG |
| | | CAGTGGCACGATCTCAGCCCACTGGAACCTCTGCCTCCCGGCTTCAA |
| | | GCAATTCTCCTACCTTGGCCGCCCA |
| 327 | patch | TGGAACCTCTGCCTCCCGGCTTCAAGCAATTCTCCTACCTTGGCCGC |
| | | CCAAGTAGCTGGGATTGCAGGCGCCCACCACCATGCCCAGCTAATTT |
| | | TTTTTTTTTTTTTTTTTAGACGGAGT |
| 328 | patch | CCCACCACCATGCCCAGCTAATTTTTTTTTTTTTTTTTTTTAGACGGAG |
| | | TTTCGCTCTTGTTACCCAGGCTGGAGTGCAAGGGCGCGATCTCGACT |
| | | CACCACAACCTCCGCCTCCTGGGT |
| 329 | patch | TGGAGTGCAAGGGCGCGATCTCGACTCACCACAACCTCCGCCTCCT |
| | | GGGTTCAAGCGATTCTCCTGTCTCAGCCTCCCAAGTAGCTGGGTTTA |
| | | CAGCCATGTGCCACCACGCCCAGCTAA |
| 330 | patch | CTGAGGTCGGGAGTTTGAGACCAGCCTGACCAACATGGAGAAACCC |
| | | CGTTTCTACTAAAAAAATACAAAATTAGCTGGGCGTGGTGGCACATG |
| | | GCTGTAAACCCAGCTACTTGGGAGGCT |
| 331 | patch | ACTTGAGGCCAGGCGCGGTGGCTCACGCCTGTAATCCCAGCACTTT |
| | | GGGAGGCTGAGGTGGGCGGATCACCTGAGGTCGGGAGTTTGAGAC |
| | | CAGCCTGACCAACATGGAGAAACCCCGTT |
| 332 | patch | AAGAAATAGCCCCTCAGAAACCCACTTGAGGCCAGGCGCGGTGGCT |
| | | CACGCCTGTAATCCCAGCACTTTGGGAGGCTGAGGTGGGCGGATCA |
| | | CCTGAGGTCGGGAGTTTGAGACCAGCCT |
| 333 | patch | GGAGGTCAGACTTCAGAAAGGACTTCCCTCTTTTATTTTTTTTTCTGA |
| | | GACGGAGTCTTGCTCTGTCGCCCAGGCTGGAGTGCAGTGGTACAAT |
| | | CTCAGCTCACTGCAACCTCCACCTCC |
| 334 | patch | CAGGCTGGAGTGCAGTGGTACAATCTCAGCTCACTGCAACCTCCACC |
| | | TCCTGGGTTCAAGCGATTCTTCTGCCTCGGCCTCCCAAGTAGCTGGG |
| | | ACTACAAGCACCTGCCACCACGCCCG |
| 335 | patch | TGAGGTCAGTTCCAGACCATCCTGGCCAATATGGTGAAACCCCGTCT |
| | | CTATTAAAAATACAAAAATAAGCCGGGCGTGGTGGCAGGTGCTTGTA |
| | | GTCCCAGCTACTTGGGAGGCCGAGGC |
| 336 | patch | AAGTCCTGGCCGGGTGTGGTGGCTCATGCCTGTAATCCCAGCACTTT |
| | | GGGAGGCCGAGGTAGGCGGATCATGAGGTCAGTTCCAGACCATCCT |
| | | GGCCAATATGGTGAAACCCCGTCTCTA |
| 337 | patch | TGGCCAGGATGGTCTGGAACTGACCTCATGATCCGCCTACCTCGGC |
| | | CTCCCAAAGTGCTGGGATTACAGGCATGAGCCACCACACCCGGCCA |
| | | GGACTTCTCTCTTGAGAAGAAAGGGAAA |
| 338 | patch | TAAAATATGACTTTCGGGGATTGTTATGGTATTAATGAGGTAGTGGCA |
| | | GGTACATAGTAAGTGCTCAATAAATGGTAGTTCCTCATATTATTGCCC |
| | | CAGACTATTGCTAACCCTTTCCTT |
| 339 | patch | TTGTAAGTGGCAGGGCTGAGATCTGAACCCAGTCTGGCTCCTGACCA |
| | | CAATGTTACACTGCCCCTCAAAATAAAATATGACTTTCGGGGATTGTT |
| | | ATGGTATTAATGAGGTAGTGGCAGG |
| 340 | patch | TTGTGGTCAGGAGCCAGACTGGGTTCAGATCTCAGCCCTGCCACTTA |
| | | CAAGTATTGCGGGTTGGTCAGCCGGGTGCAGTGGCTCACGCCTGTA |
| | | ATCCTAGCACTTTGGGAGGCCGAGGCG |
| 341 | patch | TTTTAGTAGAGACAGGGTTTCCCCATGTTGGCCAGGCTGGTCTGGAA |
| | | CTCCAGACCTTAAGTGATCCCGCCGCCTCGGCCTCCCAAAGTGCTAG |
| | | GATTACAGGCGTGAGCCACTGCACCC |
| 342 | patch | TCTCCTGTCTCAGCCTCCCGAGTAGCTGGGATTACAGGCACGTGCCA |
| | | CCACACCCAGATAATTTTTGAATTTTTAGTAGAGACAGGGTTTCCCCA |
| | | TGTTGGCCAGGCTGGTCTGGAACTC |
| 343 | patch | TGGTGGCACGTGCCTGTAATCCCAGCTACTCGGGAGGCTGAGACAG |
| | | GAGAATCACTTGAACCCAGGAAGCAGAGGTTGCAGTAAGCCGAGAC |
| | | TGTGCCACTGCACTCCAGCCTGGGCAAC |
| 344 | patch | TCTAGGGACCCCAACCCAACCCTCAATACTTTTTTTTTTTTTTTTTTTTT |
| | | TTTAGACAGAGTCTTGCTCTGTTGCCCAGGCTGGAGTGCAGTGGCAC |
| | | AGTCTCGGCTTACTGCAACCTCT |
| 345 | patch | CAACAGAGCGAGACTCCGTCTCAAAAAACAAACAAACATTGAACACC |
| | | CCAACTTCTTCTCAGCGTCTGCTTCTAGGGACCCCAACCCAACCCTC |
| | | AATACTTTTTTTTTTTTTTTTTTTTT |
| 346 | patch | AGGAGGCGGAGGTTGTAGGAAGGCGGAGATTGCAGTGAGCCAAGAT |
| | | CGCTCCACTGCACTCCAGCCTGGGCAACAGAGCGAGACTCCGTCTC |
| | | AAAAAACAAACAAACATTGAACACCCCA |
| 347 | patch | AGCGATCTTGGCTCACTGCAATCTCCGCCTTCCTACAACCTCCGCCT |
| | | CCTGGGTTCAAGCGATTCTCCTGCCTCAGCCTCCTGAGTAGCTGGGA |
| | | CTACAGGTGTGCACCACCACACCCAG |
| 348 | patch | CCTCAGCCTCCTGAGTAGCTGGGACTACAGGTGTGCACCACCACAC |
| | | CCAGCTAATTTTTGTATTTTTAGTAGAGACAGGGTTTCATCATGTTGG |
| | | CTAGGATGGTCTCGATCTCTTGACCT |
| 349 | patch | AGAGACAGGGTTTCATCATGTTGGCTAGGATGGTCTCGATCTCTTGA |
| | | CCTTGTGATCCGCCCTCCTAGGCCTCCCAAAGTGCTGGTATTACAGG |
| | | CGTGAGCCACCGCGCCCAGCTCAATG |
| 350 | patch | CTCCCAAAGTGCTGGTATTACAGGCGTGAGCCACCGCGCCCAGCTC |
| | | AATGTTTTTTTACAAATCAGCACCTGTGAAAGGAAGGGAGAGGAAAC |
| | | ATAATTGGGCAGAGGGAGAAGTCAAAC |
| 351 | patch | GTGCTCAAGCAAGGTGAGGCAATCACAGAGCTCCCCGCCCTTGACC |
| | | CAGGTTTGTCCAGCCTGTGTTGCTGTTTGACTTCTCCCTCTGCCCAAT |
| | | TATGTTTCCTCTCCCTTCCTTTCACA |
| 352 | patch | TCAGCGTCTGCCTCTGGTGCAGACAGCGGCCTCACTGGAAGTCATG |
| | | CGTTTTCTGGGCAGCCTACACCCAGTGCTCAAGCAAGGTGAGGCAAT |
| | | CACAGAGCTCCCCGCCCTTGACCCAGG |
| 353 | patch | CCTTCAAGGAAGGACTTGCCCCAAGCTGCAGGGAGTGCAGGCTGCA |
| | | GACAGGCTCCAGCTGTCAGCTCCTTCAGCGTCTGCCTCTGGTGCAG |
| | | ACAGCGGCCTCACTGGAAGTCATGCCTT |
| 354 | patch | GGAGGTCAAATGACTTGCCCAAGATAACAGGGCTAGTGTTGTAAACA |
| | | CAGAGATGTGCTGGCCAGATCAGCCTTCAAGGAAGGACTTGCCCCA |
| | | AGCTGCAGGGAGTGCAGGCTGCAGACA |
| 355 | patch | CTGTGTTTACAACACTAGCCCTGTTATCTTGGGCAAGTCATTTGACCT |
| | | CCAAAGCGCCTTGTCTGTAAAAACAGGTAAAATAGTAATGGGATTCAT |
| | | GGCATTCTTATGAGGATTAAATGA |
| 356 | patch | ACAGGTAAAATAGTAATGGGATTCATGGCATTCTTATGAGGATTAAAT |
| | | GAGGTAATATGTGTAAAGCTTTTAACGACACTGCCTGGCACATAGTAA |
| | | GCATTCAGTACACTGTAAACATGA |
| 357 | patch | TAACGACACTGCCTGGCACATAGTAAGCATTCAGTACACTGTAAACAT |
| | | GAACCATTAGTATCTCCACTTTACAACTGAGGCTTAGAGGCAAAGTAG |
| | | TTTACAAAAGGCCACAAAGTCTGA |
| 358 | patch | AAGCATTCAGTACACTGTAAACATGAACCATTAGTATCTCCACTTTAC |
| | | AACTGAGGCTTAGAGGCAAAGTAGTTTACAAAAGGCCACAAAGTCTG |
| | | ACTTCAATTCCCTTGCCCTAAGCAC |
| 359 | patch | GGACCCTTCTACCACATACTAAAATTAAAATGGCCCAGGCCGGGTGC |
| | | AGTGGCTCACACCTATGTGAGCACTTTGGGAAGCCAAGGCAGGTAG |
| | | ATTACTTGAGGTCAGGAGTTCGAGACC |
| 360 | patch | CTTTGGGAAGCCAAGGCAGGTAGATTACTTGAGGTCAGGAGTTCGAG |
| | | ACCAGCCTGGCCAACATGGTGAAACCCCATCTCTTCTAAAAATACAAA |
| | | AATTAACCCAGTGTGGTGGCATGCA |
| 361 | patch | ACCCCATCTCTTCTAAAAATACAAAAATTAACCCAGTGTGGTGGCATG |
| | | CACCTGTAATCCCAAATACTCGGGAGGCTGAGGCATGAGAATCGGTT |
| | | GAACCCGGCAGGTGGAGGTTGCAGT |
| 362 | patch | GGAGGCTGAGGCATGAGAATCGGTTGAACCCGGCAGGTGGAGGTTG |
| | | CAGTAAGCGGAGATCACACCACTGCACTCCAACCTGGGCGACAAAG |
| | | TGAGACTCAGTCTCAAAAAATAAAATGA |
| 363 | patch | CACTCCAACCTGGGCGACAAAGTGAGACTCAGTCTCAAAAAATAAAA |
| | | TGAAATAAAATAAAGTGGCTCAAAGACTCAATTTAAGAGTTAAAACTAT |
| | | AAAACTCTTAGAAGAAAATATTGT |
| 364 | patch | AGACTCAATTTAAGAGTTAAAACTATAAAACTCTTAGAAGAAAATATTG |
| | | TTGAAAATCTTCATCACATTGGACTTGGCAACAACTGCATAAATAGGA |
| | | TACCAAAAGTACAAGGAAGAAAA |
| 365 | patch | GACTTGGCAACAACTGCATAAATAGGATACCAAAAGTACAAGGAAGA |
| | | AAAAATAGTAGGTAAATTGAACTTCATCAAATTAAGAACTTTTGTGCAC |
| | | CAAAGGACACTATCAAGAAAGTGA |
| 366 | patch | TCATCAAATTAAGAACTTTTGTGCACCAAAGGACACTATCAAGAAAGT |
| | | GAAAAAACCTGGAGAATGGAAGAAAATATTTGCAGATCATATATCTAA |
| | | CAAGGGATTAATATTCAAAATATA |
| 367 | patch | AAAATATTTGCAGATCATATATCTAACAAGGGATTAATATTCAAAATAT |
| | | ATATAGAACTCCTACAACTTGACAACAAAAAAACAAACAACCCAATTC |
| | | AAAAATGGGCAAAGTACTTGAAT |
| 368 | patch | ACAACAAAAAAACAAACAACCCAATTCAAAAATGGGCAAAGTACTTGA |
| | | ATAGACACTTCTCCAAAGAAGATATACTAATGGACAATAAATTCGTGG |
| | | AAATATGCTCAACATCGTTAGTCA |
| 369 | patch | TTCTTGATTATGGCCATCCTGGTTAGTGTGAAGTGGCATCTCATTGTG |
| | | GTTGTAATCTGCATTTCCCCAATGACTAACGATGTTGAGCATATTTCC |
| | | ACGAATTTATTGTCCATTAGTATA |
| 370 | patch | GTACATTCCCACCAGCAATGCACGGAGGATTCCAATTTCTCCACATC |
| | | CCCACCAAAACTTATTATCCATTTTCTTGATTATGGCCATCCTGGTTA |
| | | GTGTGAAGTGGCATCTCATTGTGGT |
| 371 | patch | GTGACTTGGTAGTTCTATTTTTAACCTTTTGAGGAACTGCCAAACTGT |
| | | TTTCCCCAATGACTGCACTATTGTACATTCCCACCAGCAATGCACGGA |
| | | GGATTCCAATTTCTCCACATCCCC |
| 372 | patch | AAACAGTTTGGCAGTTCCTCAAAAGGTTAAAAATAGAACTACCAAGTC |
| | | ACCCAGCAATTCCATTCTTAGGCATATATTCAAAAGAAATGAAAGCAG |
| | | ATATTTGTACACCAGTGTTCACAG |
| 373 | patch | CATATATTCAAAAGAAATGAAAGCAGATATTTGTACACCAGTGTTCAC |
| | | AGCTGCACTATTTACAATAGTCAAAAGGTAGAAACAACCTAGGTCCAT |
| | | CCACAAATGAATGGATAAATAAAA |
| 374 | patch | AAAAGGTAGAAACAACCTAGGTCCATCCACAAATGAATGGATAAATAA |
| | | AACGTAGCATATACATACAATGGTACACTAGTCCGCTGTAAAAAGAAA |
| | | TTTTGATCTTACTGCATGCTACAT |
| 375 | patch | GTACACTAGTCCGCTGTAAAAAGAAATTTTGATCTTACTGCATGCTAC |
| | | ATGGCTTCGACATACTACAACATGGATGGACCTTGAAAACATTATTCT |
| | | TTGTGAAATAAACTAGACACAGGA |
| 376 | patch | TGGATGGACCTTGAAAACATTATTCTTTGTGAAATAAACTAGACACAG |
| | | GACAAATGTTAGACGATTCCACTTATATGAGGCACCTAGAATGGGCA |
| | | ATTTGGTAAGCAAAGTAGAATAGAA |
| 377 | patch | ATCCCCAACATAAACTCTGTGACCATTAAACAGTAACTCCCCATTCCC |
| | | TGCTACCTGTGCCCCTAGTAATTTCTATTCTACTTTGCTTACCAAATTG |
| | | CCCATTCTAGGTGCCTCATATAA |
| 378 | patch | TCAGTGGCATTAAGTATATTCACAATGTCGTGTACCAATCACCACTCT |
| | | TTATCCCCGAAACTGTTTCATCATCCCCAACATAAACTCTGTGACCAT |
| | | TAAACAGTAACTCCCCATTCCCTG |
| 379 | patch | AAAGAGTGGTGATTGGTACACGACATTGTGAATATACTTAATGCCACT |
| | | GAATTTTACACTTGAAGTGGTTAAAGCGATAAATATTATAGTTTGCATA |
| | | TTTTATCATAAAAATATTTTTTT |
| 380 | patch | AAAGCGATAAATATTATAGTTTGCATATTTTATCATAAAAATATTTTTTT |
| | | AAACGATGAAGGGACGTGAACGGGTTGAAATTTTATAAAAAGTGGCC |
| | | AGGGAAGGTGTCACTGCAATGGT |
| 381 | patch | CGGGTTGAAATTTTATAAAAAGTGGCCAGGGAAGGTGTCACTGCAAT |
| | | GGTGTCCTACAGGAGGAGGAAGATCATGTGGACATCTGCGGGAAGG |
| | | GTGTTCTGGCAGAGGGAGTAGCACGGG |
| 382 | patch | TCATGTGGACATCTGCGGGAAGGGTGTTCTGGCAGAGGGAGTAGCA |
| | | CGGGCGATGGCTCTGAGGACTGTGAGAAGTATAGTTGGAAACAGCG |
| | | AGGAGGCCAGGGTGTCCGAAGCTGAGTA |
| 383 | patch | CTCCTCCTCACTCTCAGCAGAAACTGACCTTCCCCCTCTTATCTCACC |
| | | TCCTCCCACTCTCTCTGGCTTACTCAGCTTCGGACACCCTGGCCTCC |
| | | TCGCTGTTTCCAACTATACTTCTCA |
| 384 | patch | CGTGCACCTGCCACTTCAGCCCCACGGGTCTATAAAATGGGCATGAT |
| | | TATCGTGGCTACCTCACTGGTCCTGGCAATTAAGGAACAATGTGTGC |
| | | CAGGCACTCTGTAAACCACATACTTG |
| 385 | patch | CCCCACGGGTCTATAAAATGGGCATGATTATCGTGGCTACCTCACTG |
| | | GTCCTGGCAATTAAGGAACAATGTGTGCCAGGCACTCTGTAAACCAC |
| | | ATACTTGCGAGTGTCAAGCTGGTGAC |
| 386 | patch | TGTCTGAAAGGACCATTGAAGGGAGCAATTCTTTTTTTTTTTTTTTTTG |
| | | AAGATGGAGTCTTGCTCTGGACTCCAGGCTGGAGTGCAGTGGTGCG |
| | | ATCTCAGCTCACTGCAACCTCCACC |
| 387 | patch | CTCCAGGCTGGAGTGCAGTGGTGCGATCTCAGCTCACTGCAACCTC |
| | | CACCTCCCAGGTTCAAGCAATTCTCTTGCCTCAGCCTCCCGAGTAGC |
| | | TGGGACTCCAGGTGCGTGCCACCACGC |
| 388 | patch | CAGGAGTTTGAGACCAGCCTGGCCAACATGGTGAAACCCCATCTCTA |
| | | CTAAAAATACAAAAATTAACTGGGCGTGGTGGCACGCACCTGGAGTC |
| | | CCAGCTACTCGGGAGGCTGAGGCAAG |
| 389 | patch | CGCTCCCTTTAGGCTGGGTATGGTGTCTCTCAGCACTTTGCGAGGCC |
| | | AAGGCGGGCAGATCATTTGAGGTCAGGAGTTTGAGACCAGCCTGGC |
| | | CAACATGGTGAAACCCCATCTCTACTA |
| 390 | patch | CTTGGCCTCGCAAAGTGCTGAGAGACACCATACCCAGCCTAAAGGG |
| | | AGCGATTCTATTCTACTATTCTTCCTTCTGCTAATCCTTCCATTCTTTA |
| | | ATTTAATAACGAAGATTTTTTGAGT |
| 391 | patch | CTTCTGCTAATCCTTCCATTCTTTAATTTAATAACGAAGATTTTTTGAG |
| | | TACCTGTCATATACCAGGTGCTGTTCTGGGCCCTGGGAATACAGCTG |
| | | TTAACAAAATCATCAAACCACTTC |
| 392 | patch | TGTTCTGGGCCCTGGGAATACAGCTGTTAACAAAATCATCAAACCACT |
| | | TCCCTCGTGGAGCCCACATTGCAGTGAGAGAGACAAACACGACACA |
| | | CACTCTCAAGTCCTTGAAGATAAAGA |
| 393 | patch | AGTGAGAGAGACAAACACGACACACACTCTCAAGTCCTTGAAGATAA |
| | | AGAAAACTGGGTAACGGAGAGAAGAGGCCAGGGTTTGTTCTATAATC |
| | | ATTAATAACACGAGCAGTAAGAAGTA |
| 394 | patch | GAGGCCAGGGTTTGTTCTATAATCATTAATAACACGAGCAGTAAGAA |
| | | GTAAAATTTATCTAAGTAACAACTTATAAAGGGTCTACTGTGTGCTAA |
| | | GCTCTCATCCAGGTTCCCAAGGATT |
| 395 | patch | TTATAAAGGGTCTACTGTGTGCTAAGCTCTCATCCAGGTTCCCAAGG |
| | | ATTAACTCAGACCACACAGTAATTGAATAGATTCTATCATTGTCATCTT |
| | | ACAGAGGCCCAGAGAGAGAAAGTG |
| 396 | patch | CTTGGCCACTAGACCCAAAGTGGCTGAGTTAGAATCCCAGCCCCGTT |
| | | ACCAGCTATGACACTAGGCAAGTCACTTTCTCTCTCTGGGCCTCTGT |
| | | AAGATGACAATGATAGAATCTATTCA |
| 397 | patch | CTCCTCTGTGAGTCCTCTGCGGACCACCCTAGGCAAGCAAAGGGATT |
| | | AGGAGCTTGGCCACTAGACCCAAAGTGGCTGAGTTAGAATCCCAGC |
| | | CCCGTTACCAGCTATGACACTAGGCAA |
| 398 | patch | GGTTCTCAGGGCTGCCTGGGTTCAAACCCCAGCTCCAGACTTTGACT |
| | | TCCTATGCACCCATTTGAACAAGGTAACCTCTCTGTGCCTCAGCTGTC |
| | | AGGAGGGTAGAGGAGAGGACTCAAT |
| 399 | patch | AGCCGAGATCCCGCCACTGCACTCCAGCCTGGGCGACAGAGCGAGA |
| | | CTCCGTCTCAAAAAAAAAGAACAAGGTTCTCAGGGCTGCCTGGGTTC |
| | | AAACCCCAGCTCCAGACTTTGACTTCC |
| 400 | patch | GCCACACCAGCCGGACCACCCGCCAGAGGCTCAACGGCCCCTCCTC |
| | | GCAGCCGAGGAAACTGAGGCTCGGAGGCAGGGCGCAGAGGCCCAA |
| | | GGTGACACAGCAGGCGGCCGTCGGCCGGG |
| 401 | patch | GGCCCGCGCTCCCCGGCCGACGGCCGCCTGCTGTGTCACCTTGGG |
| | | CCTCTGCGCCCTGCCTCCGAGCCTCAGTTTCCTCGGCTGCGAGGAG |
| | | GGGCCGTTGAGCCTCTGGCGGGTGGTCCG |
| 402 | patch | GCAGCAAATCATGGGCTGTCCTAGCCCCTTTTCAGATGAGAAAGGAG |
| | | CCCAGAGAGGGGAGAGGGCCTGCCAAGGTGGCACATCTGGCCAGG |
| | | CGCTGCGTAGGCTGAGCCCTGGGGGTCA |
| 403 | patch | AGACGTCCAGTTTTTTGGGAGGGGTGGGACACAGGAGTCTGTTTTTT |
| | | AACTAGCTCCCCAGAGCACCTGAGCAGCAAATCATGGGCTGTCCTAG |
| | | CCCCTTTTCAGATGAGAAAGGAGCCC |
| 404 | patch | AAGAAATGCAGACGTCCAGTTTTTTGGGAGGGGTGGGACACAGGAG |
| | | TCTGTTTTTTAACTAGCTCCCCAGAGCACCTGAGCAGCAAATCATGG |
| | | GCTGTCCTAGCCCCTTTTCAGATGAGA |
| 405 | patch | GAGGCTGAGGTGGGTGAATCACTTGAGGTCAGGAGTTCGAGACCAG |
| | | CCTGACCAATATGGTGAAACCCCATCTCAAAAAAAAAAAAAATTGGGG |
| | | CTAACGGTGAGCCTGTTTTGCAGGAT |
| 406 | patch | CAGGCTGGTCTCGAACTCCTGACCTCAAGTGATTCACCCACCTCAGC |
| | | CTCCCAAAGTGCTGGGATTATAGGCGTGAGCCACTGCACCCAGCCTA |
| | | GCCCCATTTTTCACATGAAGAGACCG |
| 407 | patch | AGGCTGGTCTCGAACTCCTGACCTCAAGTGATTCACCCACCTCAGCC |
| | | TCCCAAAGTGCTGGGATTATAGGCGTGAGCCACTGCACCCAGCCTA |
| | | GCCCCATTTTTCACATGAAGAGACCGA |
| 408 | patch | AGGCAGAAGTTGTGATGAGCTGAGATCCCGCCACTGCACTCCAGACT |
| | | GGGCAACAGAGCAGGACTCTGTAAAAAACAAAACAAAACAAAAAATT |
| | | ACCCTTCCCCACAATTATAAGCATTT |
| 409 | patch | CCCAGTCTGGAGTGCAGTGGCGGGATCTCAGCTCATCACAACTTCTG |
| | | CCTCCTGAGTTCAAGCGATTCTCCTGGCTCAGCCTCTCGAGTAGCTG |
| | | GGACTAGAGATGCATGCCACCACGCC |
| 410 | patch | CTGGCTCAGCCTCTCGAGTAGCTGGGACTAGAGATGCATGCCACCA |
| | | CGCCTGGCTAATTATTTGTATTTTTAGTAGAGATAGGGTTTCTCCATG |
| | | TTGGTCAGGCTGGTCTCGAACTCCTG |
| 411 | patch | TAGTAGAGATAGGGTTTCTCCATGTTGGTCAGGCTGGTCTCGAACTC |
| | | CTGACCTCAAATGATCTGCCCGCCTCGGTCTCCCAAAGTGCTGGGAT |
| | | TACAGTGAGCCACCACGCCTGTCTGG |
| 412 | patch | TTGGTCAGGCTGGTCTCGAACTCCTGACCTCAAATGATCTGCCCGCC |
| | | TCGGTCTCCCAAAGTGCTGGGATTACAGTGAGCCACCACGCCTGTCT |
| | | GGGTAATTTTTTTTTAAGTATTCTGT |
| 413 | patch | GGCTGAGCCAGGATGCTGCTGGTGCGCTCGCCGCGCCGGCCCCGC |
| | | CCTCCCGCGGCCCCGCCCCGCCCGCAGGCCCCGCCCCCGGCAGGC |
| | | CCCGCCCCGCCGAGCGCCCGGCCGCCCTCC |
| 414 | patch | CTCGCCGCGCCGGCCCCGCCCTCCCGCGGCCCCGCCCCGCCCGCA |
| | | GGCCCCGCCCCCGGCAGGCCCCGCCCCGCCGAGCGCCCGGCCGC |
| | | CCTCCTGTCTGGGCGCGCGTGGCTGGCGGGT |
| 415 | patch | AGAATGATTGTTGTCGTAAAGGCCTTTGATAATAATAGATACTGTTTA |
| | | CCCAGTGCTTGATGCCAGGCATTGCTACAAACACTGCGTACACATTA |
| | | TTCTATTTAATCTTCATAACCCAGT |
| 416 | patch | TCACTAGCATTTAACCTCTGTGCCCCAGCTTCCTCTTCTGTGAAAGGA |
| | | CAGTAATGACAGTACCTACCTCACTGGGTTATGAAGATTAAATAGAAT |
| | | AATGTGTACGCAGTGTTTGTAGCA |
| 417 | patch | GTGCCAAGTGTGGTACTGCTCTCACTAGCATTTAACCTCTGTGCCCC |
| | | AGCTTCCTCTTCTGTGAAAGGACAGTAATGACAGTACCTACCTCACTG |
| | | GGTTATGAAGATTAAATAGAATAAT |
| 418 | patch | TGTGGAGGGAGGTGGGAAGTTGTGTGGTGTATTGTTTTGAAGGGGTG |
| | | CTGGAACGAAGTACCACACACTAAGTGGCTTCAACAACCGAAATGTA |
| | | TGGTCTGTCCTGGTTCTGGAGACTAG |
| 419 | patch | AGTGGCTTCAACAACCGAAATGTATGGTCTGTCCTGGTTCTGGAGAC |
| | | TAGAAGTACAAAATCACGGCGTGGGAGGGCTGGCTGCTTCCGAGGC |
| | | CCATGAAAGAGCATGTGTGCCCGGCCA |
| 420 | patch | GGAGGGCTGGCTGCTTCCGAGGCCCATGAAAGAGCATGTGTGCCCG |
| | | GCCACTCTGACTTGCAGATGCCCATCTTCTCCCTATGTCTCCTCACAC |
| | | CATCTTCCCTGTGGTGTGTGTGTGCA |
| 421 | patch | TCTTCTCCCTATGTCTCCTCACACCATCTTCCCTGTGGTGTGTGTGTG |
| | | CAAATCCCTTCTTTCTATAAGGACACCAGTCATACTGGACTAGGGCC |
| | | CACCCGAATTGCCTCATTTTAACTT |
| 422 | patch | AGAGGAGTGATAGTGGAGTAGGGGGGAGGGGAATTGGGTGATTTTAA |
| | | CTTGATGACCTCTGTAATCATGAGGTAATGTCCAAGTAAGGTCCCATT |
| | | CTAAGATACCTGAATGCCCAGGCAC |
| 423 | patch | GGTAATGTCCAAGTAAGGTCCCATTCTAAGATACCTGAATGCCCAGG |
| | | CACGGTGGTTCATGCCTGTAATCCCAGCACTTTGGGAGGCCGAGGC |
| | | AGGCAGATCAACTGAGGTCAGGAGTTC |
| 424 | patch | GGAGGATGGGGGGGTAATTTTTTTATTTTTAGTAGAGAGAGGGTTTGA |
| | | CCATGTTGGCCAGGCTGGTCTTGAACTCCTGACCTCAGTTGATCTGC |
| | | CTGCCTCGGCCTCCCAAAGTGCTGG |
| 425 | patch | CCTCCACCTCCCTGGGTTCAAACAATTCTTCTGCCTCAGCCTCCCGA |
| | | GTAGCTGGGACTACAGGTACGCACCACCATGCCCCGCTAATTTTTTT |
| | | ATTTTTAGTAGAGACAGGGTTTCACC |
| 426 | patch | TACTCGGGAGGCTGAGGCAGAAGAATTGTTTGAACCCAGGGAGGTG |
| | | GAGGTTGCAATGAGCCAAGATCACGCCACTGCATTCCAGCCTGGGA |
| | | GACAGAGCAAGACTCTGTCTCAAAACAA |
| 427 | patch | TTACGGGTTGAATTGTCACCCCCACATTCCCCAAGTTCATATGTTCAG |
| | | GGGTTTGTTGTTGTTGTTGTTGTTGTTTTGAGACAGAGTCTTGCTCTG |
| | | TCTCCCAGGCTGGAATGCAGTGGC |
| 428 | patch | GGTGAGACAGCCAGTGACGACCATCCACATGTTACGGGTTGAATTGT |
| | | CACCCCCACATTCCCCAAGTTCATATGTTCAGGGGTTTGTTGTTGTTG |
| | | TTGTTGTTGTTTTGAGACAGAGTCT |
| 429 | patch | AGGCTGGGAAAGGCACACAACCCAGTATCAGACCTGAGTTCACACTC |
| | | CTTCTGGCAGGAACTTCTAGGTGACATTGGGAAGCAACTTCATTCCTT |
| | | AGAACCTAGATTTCTCCTCAGGCTA |
| 430 | patch | AAAGAGACCTAAGAGATTATTTAGCCAAGCCCCCTTATAGCCTGAGG |
| | | AGAAATCTAGGTTCTAAGGAATGAAGTTGCTTCCCAATGTCACCTAGA |
| | | AGTTCCTGCCAGAAGGAGTGTGAAC |
| 431 | patch | AGGTATTATTCCTGATTTAAGGAGGATGCAATGAGGCTTGATGTGGAT |
| | | TCACCCACCCCAAAGCCTGTGTTCTTAACCACTATGCAATACTGCCTC |
| | | CCATCAAAGTCTCTATCTCAAGTC |
| 432 | patch | ATTTAATGAGAACGCCAAGGGCGTGGCTGTCAATCCTATGACACCTG |
| | | TCTGAGGTAGGTATTATTCCTGATTTAAGGAGGATGCAATGAGGCTT |
| | | GATGTGGATTCACCCACCCCAAAGCC |
| 433 | patch | GCATGAACCCAGGAGGTAGAGATTGCAGTGAGCCGAGATCGCACCA |
| | | CTGCACCCCAGCCTGGGCAACAGAGTGAGACTCCGTCTCAAAAAAA |
| | | GAGACTGGTTTCTTACCTAAGTCACAAA |
| 434 | patch | GCAGTGGTGCGATCTCGGCTCACTGCAATCTCTACCTCCTGGGTTCA |
| | | TGCCATTTTCCTGCCTCAGCCTCCCAAGTAGCTGGGACCACAGGCAC |
| | | CCACCACCACACCCGGCTAGTTTTTT |
| 435 | patch | CTcGGCTCACTGCAATCTCTACCTCCTGGGTTCATGCCATTTTCCTGC |
| | | CTCAGCCTCCCAAGTAGCTGGGACCACAGGCACCCACCACCACACC |
| | | CGGCTAGTTTTTTGTATTTTTAGTAG |
| 436 | patch | AGCCTGGCCAACATGGTGAAACCCCCTCTCTACTAAAAATACAAAAAT |
| | | TAGCTGGGCATGGTGGTGGGTGCCTATAATCCCAGCTACTCGGGAA |
| | | GCTGAGGCAGGACAATCATTTGAACC |
| 437 | patch | GGTGCAGTGGCTCATGCCTGTAATCTCAGCACTTTGGGAGGCCGAG |
| | | GCAGGCGGTCAAGAGTTCGAGACCAGCCTGGCCAACATGGTGAAAC |
| | | CCCCTCTCTACTAAAAATACAAAAATTA |
| 438 | patch | CTGCCTCGGCCTCCCAAAGTGCTGAGATTACAGGCATGAGCCACTG |
| | | CACCAGGCCTGTCTTCAGGTCTTTTTTATTTTATTATTTATTTATTTATT |
| | | TATTTATTTATTTATTTATTTTGA |
| 439 | patch | TTTATTTTATTATTTATTTATTTATTTATTTATTTATTTATTTATTTTGAGA |
| | | CAGAGTCTCGCTCTGTCGCCCAGGCTGGAGTGCCGTGGTGCGATCT |
| | | CGGCTCACTGCAAGCTCCACCT |
| 440 | patch | CCCAGGCTGGAGTGCCGTGGTGCGATCTCGGCTCACTGCAAGCTCC |
| | | ACCTCCCGGGTTCACGCCATTCTCCTGTCTCAGCCTCCCGAGTAGCT |
| | | GGGATTACAGGCGCCTGCCACCATGCC |
| 441 | patch | AGGAAATCGAGACCATCCTGGCCAACTTAGTGAAACCCCATCTCTAC |
| | | TAAAAATACAAAAAAATTAGCTGGGCATGGTGGCAGGCGCCTGTAAT |
| | | CCCAGCTACTCGGGAGGCTGAGACAG |
| 442 | patch | GGCCGGGTGCAGTGGCTCATGCCTGTAATCCCAGCACTTTGGGAGG |
| | | CCGAGGCAGGCAGATCACGAGGTCAGGAAATCGAGACCATCCTGGC |
| | | CAACTTAGTGAAACCCCATCTCTACTAA |
| 443 | patch | TCGGCCTCCCAAAGTGCTGGGATTACAGGCATGAGCCACTGCACCC |
| | | GGCCCACATACTACATTTTCTATCCACTCACCAGTTGATGAAGACTTA |
| | | ATTTGTTTCCAGTTTTCTTTTTTCTT |
| 444 | patch | CACTCACCAGTTGATGAAGACTTAATTTGTTTCCAGTTTTCTTTTTTCT |
| | | TTTTTTTGAGATGGGATCTCCCTGTATTGCCCAGGTTGGTCTTGAACT |
| | | CCTGGGCTCAAGTGATCCTCCCA |
| 445 | patch | TGAGATGGGATCTCCCTGTATTGCCCAGGTTGGTCTTGAACTCCTGG |
| | | GCTCAAGTGATCCTCCCACCTAAGCCTCCCAAAGTGCTGGGATTTGT |
| | | TTCCGGTTTTTTTCCTACTATGCTAT |
| 446 | patch | TTTTTTTTTTAACCTCTGTACCCCTAGCTTTTTTTTTTTTTTTTTTTTTTT |
| | | TTGAGATGGATTCTTGCTCTGTCGCCCAGGCTGGAGTGCAGCAGTGT |
| | | GATCTTGGCTCACTGCAACCTC |
| 447 | patch | TGTCGCCCAGGCTGGAGTGCAGCAGTGTGATCTTGGCTCACTGCAA |
| | | CCTCCACCCCCCGGGTTCAAGCAATTCTCCTGCCTCAGCCTCCCAAG |
| | | TAGCTGGGATTACAGGCGTACGCCACC |
| 448 | patch | TTGAGACCAGCCTAGCCAACATGGTCAAACCCCGTCTCTACTAAAAA |
| | | AAATACAAAAATTAGCCAGGCATGGTGGCGTACGCCTGTAATCCCAG |
| | | CTACTTGGGAGGCTGAGGCAGGAGAA |
| 449 | patch | CAGTGGCTCACACCTGTAATTCCCAGCACTTTGGGAGGCTGAGGCA |
| | | GGCGGATCACCTGAGGTTAGGAGTTTGAGACCAGCCTAGCCAACAT |
| | | GGTCAAACCCCGTCTCTACTAAAAAAAA |
| 450 | patch | CGCCTGCCTCAGCCTCCCAAAGTGCTGGGAATTACAGGTGTGAGCC |
| | | ACTGTGCTCAGCCTGTGTGTTTTGAGACAGGGTCTTGCTTCGTCACC |
| | | CAGGCTGGAGTGCAGTGGCTCAATCAC |
| 451 | patch | AGACAGGGTCTTGCTTCGTCACCCAGGCTGGAGTGCAGTGGCTCAA |
| | | TCACAGCTTACTGCAGCCTGGACTTCCCAGGCTCAAGTGATCCTCCC |
| | | GCCTCAGCCTCTCAAGTAGCTGGAACT |
| 452 | patch | TCCCAGGCTCAAGTGATCCTCCCGCCTCAGCCTCTCAAGTAGCTGGA |
| | | ACTACAGGCGAGTGCCACCATGCCTGGCTAAGTTTTAATTTTATGTAG |
| | | AGATGGTCTCAGTATGTTGCCCAGG |
| 453 | patch | CTGGCTAAGTTTTAATTTTATGTAGAGATGGTCTGAGTATGTTGCCCA |
| | | GGCTGATTTCAAACTCTTGGGCCCAAGTGATCCTCCTGCCTTGCTTC |
| | | CCTCCCAAAGTGCTAGGATTACAGG |
| 454 | patch | CCAAGTGATCCTCCTGCCTTGCTTCCCTCCCAAAGTGCTAGGATTAC |
| | | AGGCATGAACCACTTCACCTGGCCCATGCTATATGTTTGCCCCTCGT |
| | | CTTCTTACCTACTCCCTCAGTTTTCT |
| 455 | patch | CTTCAGCACAGGAGTTTGGGGAAGACACAAACATTCAACCCCTACTG |
| | | TTCCAACCCCTGCTTCACAGATGAGAAAACTGAGGGAGTAGGTAAGA |
| | | AGACGAGGGGCAAACATATAGCATGG |
| 456 | patch | ATTAATAAGGGCTCCACCCACATCACCTGATGACCTCCCAGAGGTCC |
| | | TACCTCCAAATACCATCACTCAGCTTCAGCACAGGAGTTTGGGGAAG |
| | | ACACAAACATTCAACCCCTACTGTTC |
| 457 | patch | GTAGGACCTCTGGGAGGTCATCAGGTGATGTGGGTGGAGCCCTTAT |
| | | TAATGGGATTTGTGCCCTTATAGAAAGGGACACAACATGAGATGATCT |
| | | TGCTCTGGGCCATGCCGGGAGGAAGG |
| 458 | patch | AAGGGACACAACATGAGATGATCTTGCTCTGGGCCATGCCGGGAGG |
| | | AAGGGAGAAGGTGGCTGTCAACGAGCCAGGAAGCGGCACTCCTGAC |
| | | ACTGGATCTACCAACACCCCTATCTCAG |
| 459 | patch | GCCAGGAAGCGGCACTCCTGACACTGGATCTACCAACACCCCTATCT |
| | | CAGACTTCCAGCCTCCAGGACCGTGAGAACTGTTTCCTGTTGAAGCC |
| | | ACCCTCTCTATATTTGTTACAGCAGC |
| 460 | patch | TGAGAACTGTTTCCTGTTGAAGCCACCCTCTCTATATTTGTTACAGCA |
| | | GCCCACACTGATTAAGACACTTTTTTTTTTTTTTTTTTTTTTTTTTTTTTT |
| | | GAGATGGAGCCTCGCTCCATC |
| 461 | patch | TTTTTTTTTTTTTTTTTTTTTTTTTTTTTGAGATGGAGCCTCGCTCCATC |
| | | ACCCAGGCTGGAGTGCAATGGCGCAATCTCGGCTCACTGCAACCTC |
| | | TGCCTCCCGGGTTCAAGTGATTAT |
| 462 | patch | GCGCAATCTCGGCTCACTGCAACCTCTGCCTCCCGGGTTCAAGTGAT |
| | | TATCCTGGGTCAGCCTCCCAAGTAGCTGGGATTGCAGGCACCTGCC |
| | | ACCATGGCTGGCTAAATTTTGTATTTT |
| 463 | patch | AGCTGGGATTGCAGGCACCTGCCACCATGGCTGGCTAAATTTTGTAT |
| | | TTTTAGTAGAGACAGGGTTTCACCATGTTGGCCAGGCTGGTCTCCAA |
| | | CTCCTGACCTCAGGTGATCCACCTGC |
| 464 | patch | CATGTTGGCCAGGCTGGTCTCCAACTCCTGACCTCAGGTGATCCACC |
| | | TGCCTCGGCCTCCCAAAGTGCTGGGCGTGAGCCACCATGCCCGGCC |
| | | TGACACTTATCTTTTATATCTATATAT |
| 465 | patch | GGCGTGAGCCACCATGCCCGGCCTGACACTTATCTTTTATATCTATAT |
| | | ATTTTTTTCTTTGTTGAGACGGAGTCTCACTGTGTCGCCCAGGCTGGA |
| | | GTGCAGTGGTGTGATCTTGGCTCA |
| 466 | patch | AGTCTCACTGTGTCGCCCAGGCTGGAGTGCAGTGGTGTGATCTTGG |
| | | CTCACCGAAACCTCTGCCTCCCAGGTTCAAGTGATTCTCGTGCCTCA |
| | | GCCTCCGGAGCAGCTGGGATTACAGGC |
| 467 | patch | GTTCAAGTGATTCTCGTGCCTCAGCCTCCGGAGCAGCTGGGATTACA |
| | | GGCGTGAGCCACCATGACTGGCCTAAAACACCTACCTTTTATTTTTTA |
| | | AAAAACCAACACATTATCAGTACTT |
| 468 | patch | TAAAACACCTACCTTTTATTTTTTAAAAAACCAACACATTATCAGTACT |
| | | TCATGCCAGACACCATTCTAGGAAATTAACTCCTCTGACTTTCACATC |
| | | AATCCCATGGGTAGCTGTTATTT |
| 469 | patch | GAAATTAACTCCTCTGACTTTCACATCAATCCCATGGGTAGCTGTTAT |
| | | TTACAGATGAGGAAACTGAGGCACAGAGGTAAAACAACTTTGCTGAG |
| | | GTCACCTTGCTGCTAAGTGACACAG |
| 470 | patch | ACAGAGGTAAAACAACTTTGCTGAGGTCACCTTGCTGCTAAGTGACA |
| | | CAGCTGGCATCTAGTGCAGCAGGCTGGCTCCAAGTCTATGCCCCTCA |
| | | CCCCTCAGACGCTGCTCACCAAGCCG |
| 471 | patch | AGCCCCTCCGGCTTGGTGAGCAGCGTCTGAGGGGTGAGGGGCATA |
| | | GACTTGGAGCCAGCCTGCTGCACTAGATGCCAGCTGTGTCACTTAGC |
| | | AGCAAGGTGACCTCAGCAAAGTTGTTTT |
| 472 | patch | ATCTTTATTTTTATTAAAGGACAGGGTCTCTGGCCTGGTGCGTTGGCT |
| | | CACGCCTGTAATCCCAGCACTTTGGGAAGCCAAGGCGGGCGGATCA |
| | | CGAGGTCAGGAGATTGAGACCATCCT |
| 473 | patch | TGGGAAGCCAAGGCGGGCGGATCACGAGGTCAGGAGATTGAGACCA |
| | | TCCTGGCCAACACGATGAAACCCCGTCTCTACTAAAAATACAAAAATT |
| | | AGCTGGGCGTGGTGGTGAGTGCCTGT |
| 474 | patch | GTCTCTACTAAAAATACAAAAATTAGCTGGGCGTGGTGGTGAGTGCC |
| | | TGTAGTCCCTGCTACTCAGGAGGCTGAGGCAGGAGACTTGCTCCTG |
| | | AACCCAAGAGGCGGAGGTTGCAGTGAG |
| 475 | patch | CTGAGGCAGGAGACTTGCTCCTGAACCCAAGAGGCGGAGGTTGCAG |
| | | TGAGCCAAGATCCCGCCACTGCACTCCAGCTTCCCGACAGAGCGAG |
| | | ACTCTGTCTCACAAAAAAAAAAAAAAAA |
| 476 | patch | TCCAGCTTCCCGACAGAGCGAGACTCTGTCTCACAAAAAAAAAAAAA |
| | | AAAAAAAAAAAAAAGTCAGGGTCTCACCCTGTCCGCCAGGCTTGACT |
| | | GCAGTGGCGTGACCTCAGCTTACTGC |
| 477 | patch | TCACCCTGTCCGCCAGGCTTGACTGCAGTGGCGTGACCTCAGCTTAC |
| | | TGCAGCCTCAACCTCCCAGACTCAAGTGATCCTCCCACCTCAGCCTC |
| | | CTGAGTAGCTGGGACTACAGGTGCAC |
| 478 | patch | GATACCAGCCTGGACAACATAGCAAGACCCCATCTCTAAAAAAATATA |
| | | AAAATTAGCCAGGCATAGCAGTGTGCACCTGTAGTCCCAGCTACTCA |
| | | GGAGGCTGAGGTGGGAGGATCACTT |
| 479 | patch | CGTGACTCACATCTGTGATCCCAGCACTTTGGCAGGCTGAGGCAGG |
| | | AGGATCACTGGAGGCCAGGAGCTTGATACCAGCCTGGACAACATAG |
| | | CAAGACCCCATCTCTAAAAAAATATAAA |
| 480 | patch | CAGGCTGGTATCAAGCTCCTGGCCTCCAGTGATCCTCCTGCCTCAGC |
| | | CTGCCAAAGTGCTGGGATCACAGATGTGAGTCACGTGCTTGGCCTAA |
| | | ACTTTTAAATGATGAGACTTAAGTCA |
| 481 | patch | CTCAGTTTCCCCTTTTGTAAAATAGGATGATGATACTTGCACCTCAAG |
| | | GTGCTGGGAGGATTCACTGTGAGCATGTGAGAAGCAGAGGGCAGAC |
| | | TGTGGTGGCTGGTGGGCCAGGGCAGA |
| 482 | patch | CACACCTGCGCTCCAGCCTGGGTGACAGAGCGAGACTCCGTCTCAA |
| | | AAAAAAATAAAAATAAAAAATAAACTCAGTTTCCCCTTTTGTAAAATAG |
| | | GATGATGATACTTGCACCTCAAGGT |
| 483 | patch | TTTTTTGAGACGGAGTCTCGCTCTGTCACCCAGGCTGGAGCGCAGGT |
| | | GTGCGATCTCGGCTCACTGCAACCTCCACCTCCTGGGTTCAAGCGAT |
| | | TCTCCTGCCTCAGCCTCCTGAGTAGC |
| 484 | patch | CGGTGAAACCCCATCTCTACTAAAAATACAAAAAATTAGCTGGGTGTG |
| | | GTGGTGTGTGCCTGTAGTCCCAGCTACTCAGGAGGCTGAGGCAGGA |
| | | GAATCGCTTGAACCCAGGAGGTGGAG |
| 485 | patch | ATCCCAGCACTTTGGGAGGCCGAGGCGGTCGGATCACGAGGTCAGG |
| | | AGATCAAGACCATCCTGGCTAACACGGTGAAACCCCATCTCTACTAA |
| | | AAATACAAAAAATTAGCTGGGTGTGGT |
| 486 | patch | CCTGGGCAAATCACTTCCTTTCTTTAAATTCAGTTTCCCCTTTTGCCG |
| | | GGCGCGGTGGCTCATGCTTGTAATCCCAGCACTTTGGGAGGCCGAG |
| | | GCGGTCGGATCACGAGGTCAGGAGAT |
| 487 | patch | GGGTCTGCAGGCTGGGTGGACCCCAAGCTTAGTTGGATCCTGGGCA |
| | | AATCACTTCCTTTCTTTAAATTCAGTTTCCCCTTTTGCCGGGCGCGGT |
| | | GGCTCATGCTTGTAATCCCAGCACTT |
| 488 | patch | CCCGGCCTGGGGGGTCAGGAGGGCTTCCTGGAAGAGGGGGTATCC |
| | | TGAGCCCTGGAAGAGGAGACACCAGCCAGGCTGCTAGAGGCTGGG |
| | | GATCCCCAGCACACAGGCTCCAGGCTGGGC |
| 489 | patch | TAGCAGCCTGGCTGGTGTCTCCTCTTCCAGGGCTCAGGATACCCCCT |
| | | CTTCCAGGAAGCCCTCCTGACCCCCCAGGCCGGGCAGCCACTCCAG |
| | | GGCTCCTCAGTTCAGCAATGTGTCTCT |
| 490 | patch | TTCAAGTGATTCTTCTTGCCTCAGCCTCCCAAGTAGCTGGGATTACAG |
| | | GCACCCACGACCATGCCCGGCTAATTTTTGTATTTTTAGTAGAGACAG |
| | | GGTTTCACCATATTGGCCAGGCTG |
| 491 | patch | AATTTTTGTATTTTTAGTAGAGACAGGGTTTCACCATATTGGCCAGGC |
| | | TGGTCTCGAACTCCTGACCTCGTGATCCTCCTGCCTTGGCCTCCCAA |
| | | AGTGTTGGGATTACAGGCATGAGCC |
| 492 | patch | TGATCCTCCTGCCTTGGCCTCCCAAAGTGTTGGGATTACAGGCATGA |
| | | GCCACCGTGCCCGGCCTAATTTTTGTATTTTTTAGTAGAAACAGGGTT |
| | | CCACCATGTTGGTCAGGCTGGTCTT |
| 493 | patch | TGTATTTTTTAGTAGAAACAGGGTTCCACCATGTTGGTCAGGCTGGTC |
| | | TTGAGGTCCTGACCACAGGTGATCTGACCTCGCCTTGGCCTCGAAAA |
| | | GTGCTGGGATTATAGGCATGAGCCA |
| 494 | patch | TCTGACCTCGCCTTGGCCTCGAAAAGTGCTGGGATTATAGGCATGAG |
| | | CCACCGCACCCCACACCAACCTAGAAAAGTTCAACTCATTTGTCTAA |
| | | GCCCAGCAAAATCATCCAGAAGCCGG |
| 495 | patch | GAAAAGTTCAACTCATTTGTCTAAGCCCAGCAAAATCATCCAGAAGCC |
| | | GGTGGCGGTGGCTCACACCCATAATGCCAGCACTTTGGGAGGCTAA |
| | | GACATGAGGATCACTTGAGGCCAAGA |
| 496 | patch | AATGCCAGCACTTTGGGAGGCTAAGACATGAGGATCACTTGAGGCCA |
| | | AGAGTTTGAGACCAGCCTGAGAAATATAGCAAGACCCCCGTCTCTAT |
| | | GAAAAATACAAAAATTAGTTGGGTGC |
| 497 | patch | ATATAGCAAGACCCCCGTCTCTATGAAAAATACAAAAATTAGTTGGGT |
| | | GCGGCGGCATGTGCCTGTGGTCCCAACTACTCTGGAGACTGAGGCA |
| | | GGAGGATCACTTGAGGCCAGGAGCTT |
| 498 | patch | CCAACTACTCTGGAGACTGAGGCAGGAGGATCACTTGAGGCCAGGA |
| | | GCTTGAGGCTGCAGTGATCTGTGATGGTGCCACTGCACTCCAGCCA |
| | | GGGCGACAGAATGACACTGTCTCAAAAA |
| 499 | patch | TGGTGCCACTGCACTCCAGCCAGGGCGACAGAATGACACTGTCTCA |
| | | AAAAAAAATGTCGGCTGGGCGCGGTGGCTCACACCTGTAATCCCAG |
| | | CACTTAAGGAGGCTGAGGTGGGCGGATC |
| 500 | patch | TGGCTCACACCTGTAATCCCAGCACTTAAGGAGGCTGAGGTGGGCG |
| | | GATCACGAGGTCAGGAGATCGAGACCATCCTGGCTAACACGGTGAA |
| | | ACCCCGTCTCTACTAAAAATACAAAAAA |
| 501 | patch | CCATCCTGGCTAACACGGTGAAACCCCGTCTCTACTAAAAATACAAAA |
| | | AATTAGCCTGGCGTGGTGGCGGGCGCCTGTAGTCCCAGCTACTCGG |
| | | GAGGCTGAGGCAGGAGAATGGCATGA |
| 502 | patch | GCGCCTGTAGTCCCAGCTACTCGGGAGGCTGAGGCAGGAGAATGGC |
| | | ATGAACCCGGGAGGCGGAGCTGGCAGTGAGCCGAGATCGTGTCACT |
| | | ACACTCTAGCCTGGATGACAGGGCCAGA |
| 503 | patch | AGGCGGAGCTGGCAGTGAGCCGAGATCGTGTCACTACACTCTAGCC |
| | | TGGATGACAGGGCCAGACTCCATCTCAAAAAAAAAAAAAAAAAAATCA |
| | | TCTAGTGGTGATGGGACTTTGTAGGG |
| 504 | patch | GGAGAATTACAGCCCCCATCATAGTGTGTTGGGGTAAGAATTAAGTG |
| | | AGTTCCCATATATGGGACAAACTTCCCCCCCTTATGACCTGTAAACCC |
| | | AGTTTCTGGCGGCCAAGGATGGGGA |
| 505 | patch | GCACTCCAGCCTGGGTGACAGAGTGAGACTCCATCTCAAAAATAAAA |
| | | TCAATAAAATAAAGTAAAATGAGGGAGAATTACAGCCCCCATCATAGT |
| | | GTGTTGGGGTAAGAATTAAGTGAGT |
| 506 | patch | TGATTTTATTTTTGAGATGGAGTCTCACTCTGTCACCCAGGCTGGAGT |
| | | GCAATGGCATGAACTTGGCTCACTGCAACCTCTGACTCCCAGGTTCA |
| | | AGAGATTCTCTTGCCTCAGCCTCCT |
| 507 | patch | CTGCAACCTCTGACTCCCAGGTTCAAGAGATTCTCTTGCCTCAGCCT |
| | | CCTCAGTAGCTGGAACCACAGGCGCCCGCCACCACACCTGGCTAAT |
| | | TTTTGTATTTTTAGTACAGACTGGGTT |
| 508 | patch | GCCCGCCACCACACCTGGCTAATTTTTGTATTTTTAGTACAGACTGGG |
| | | TTTCACCGTGTTGGCCAGGCTGGTCTCAAACTCCTGACTTCAAGTGA |
| | | TCCGCCTGCCTTGGCCTCCCAAAGT |
| 509 | patch | GTCTCAAACTCCTGACTTCAAGTGATCCGCCTGCCTTGGCCTCCCAA |
| | | AGTGCTAGGATTACAGGTGTGAGCCACCATGTCCAGTCCTCTCCCCA |
| | | TTTTATAGGTGAGGAGACTGAGGCTC |
| 510 | patch | CCACCATGTCCAGTCCTCTCCCCATTTTATAGGTGAGGAGACTGAGG |
| | | CTCAGAGAAGTAAAGCTACTCACCTAAGATCCCACAGCCTTCAAGCG |
| | | GCAGAGCTGGGATTTGAACCTCGGCC |
| 511 | patch | TACTCACCTAAGATCCCACAGCCTTCAAGCGGCAGAGCTGGGATTTG |
| | | AACCTCGGCCATCTGGGTGTGGAGCCTTATTATTCACCACTTTGGCA |
| | | GGTGTCTCAGTGGCTTACTTACCCTT |
| 512 | patch | CCCAGCTCATGCAGTCATTTATTCATTCAACAAGCATCACCCACGCTG |
| | | GGCCCCCAAGAGGCCTCAGCCCTGACCAGGTCCTCAAGGAGCTATC |
| | | TTAAGAGAGGACAGAGCTGGACATAG |
| 513 | patch | TGACCAGGTCCTCAAGGAGCTATCTTAAGAGAGGACAGAGCTGGACA |
| | | TAGACACTCCTAGTCCCATGCAGTTAAGGAAGGTCCAGAGGGAAAGA |
| | | CAGGGTCCTGGAAGGCTTCCTGGAGG |
| 514 | patch | CCAATGATAGGTTTGGGGAATCTTAGTGAACTCCTACTCATGCGTTGA |
| | | AGCCCCAACTGTAATGCCTCCTCCTCCAGGAAGCCTTCCAGGACCCT |
| | | GTCTTTCCCTCTGGACCTTCCTTAA |
| 515 | patch | TTGGAGAGCCAATGATAGGTTTGGGGAATCTTAGTGAACTCCTACTC |
| | | ATGCGTTGAAGCCCCAACTGTAATGCCTCCTCCTCCAGGAAGCCTTC |
| | | CAGGACCCTGTCTTTCCCTCTGGACC |
| 516 | patch | CCTCTGTCCTTCACATCGCCAAACTCCAAGAAATGGTCAAAGGCTTT |
| | | GAACGGAGACCTTGAAGAAAAAGCGGGAAACTGCTTTTCAACATGTC |
| | | CAGAGATGCTCATTCTTGCCAGGTGC |
| 517 | patch | CGGGAAACTGGTTTTCAACATGTCCAGAGATGCTCATTCTTGCCAGG |
| | | TGCAGTGGCTTACACCTGTAATCCCAACACTTTGGGAGGCTGAAGTG |
| | | AGCAGATCGCTTGAGCCCAGGAGTTC |
| 518 | patch | CCAACACTTTGGGAGGCTGAAGTGAGCAGATCGCTTGAGCCCAGGA |
| | | GTTCGAGACCAGCCTGGCCATCATGGTGAAACCCCGTCTCTACTTTA |
| | | AAAAATACAAAAATTAGCCAGGTGTGG |
| 519 | patch | GGTGAAACCCCGTCTCTACTTTAAAAAATACAAAAATTAGCCAGGTGT |
| | | GGTGGTATGTGCCTGTAGTCCCAGCTACTCAGGAGGCTGAGGCAGG |
| | | AGAATTGTTTGAACCCAGGAGGTGGA |
| 520 | patch | AGCTACTCAGGAGGCTGAGGCAGGAGAATTGTTTGAACCCAGGAGG |
| | | TGGAGGCTGCAGTGAGCCAAGATCACTCCACTGCATTCCAGCCTGG |
| | | GCAACAGAGTGAGACCTGTCTCAAAAAA |
| 521 | patch | ACTCCACTGCATTCCAGCCTGGGCAACAGAGTGAGACCTGTCTCAAA |
| | | AAAAAAAAAAAGTTCACACTTAATAAGAAAACTGCAAATCACAAACTG |
| | | TTTGTCTTCACCAATCAAATTGGCA |
| 522 | patch | CACCAGTGCTACACCATGGACGCACCCCACAGCCTGGCTGGCACAG |
| | | AATGTCATTGCATTGTTGGATTTTTGCCAATTTGATTGGTGAAGACAA |
| | | ACAGTTTGTGATTTGCAGTTTTCTTA |
| 523 | patch | CAGAAGTGGACTTGCTGTGTCAGCAGGCACATGAGTTTATCATTCCG |
| | | GTGGCCTGTGCCAGGGTGCCCTCCACCAGTGCTACACCATGGACGC |
| | | ACCCCACAGCCTGGCTGGCACAGAATG |
| 524 | patch | CACCGGAATGATAAACTCATGTGCCTGCTGACACAGCAAGTCCACTT |
| | | CTGGGAATCCCTGCCTGAATATTTGCATGTTTACAGTGTGAACTGACT |
| | | AACGAATAAAAAAATGTGGCTCAGC |
| 525 | patch | CTGACCTCGTGATCCGCCCACCTTGGTCCCCCAAAGTGCTGGGATTA |
| | | CAGGCATGAGCCACCACGTCCAGGCTGAGCCACATTTTTTTATTCGT |
| | | TAGTCAGTTCACACTGTAAACATGCA |
| 526 | patch | TGCCCAGCTAATTTTTGTATTTTTAGTGGAGAGGGGGTTTCACCATGT |
| | | TGGCCAGGCTGGTCTGGTACTCCTGACCTCGTGATCCGCCCACCTT |
| | | GGTCCCCCAAAGTGCTGGGATTACAG |
| 527 | patch | CAACATGGTGAAACCCCGTCTCCACTAAAAATACAAAAATTAGCTGG |
| | | GCATGGTGGCACGTGCCTGTAATCCCAGCTACTAGGGAGGCTGAGG |
| | | CAGGAGAATCGCTTGAACCCAGGAGGC |
| 528 | patch | CCCAGCTACTAGGGAGGCTGAGGCAGGAGAATCGCTTGAACCCAGG |
| | | AGGCAGAGGTTGCAGTGAGCTGAGATCATGCCACTGCACTCCAGTTT |
| | | GGGCGTTAGAGCGAGACTCCATCTCAA |
| 529 | patch | ATCATGCCACTGCACTCCAGTTTGGGCGTTAGAGCGAGACTCCATCT |
| | | CAATAATAATAATAATAATAATAATAATAATAATTTAAAAATTAGCTGGG |
| | | CATGGTGGCTTCAGCCTGTAGTC |
| 530 | patch | ATAATAATAATTTAAAAATTAGCTGGGCATGGTGGCTTCAGCCTGTAG |
| | | TCCCAGCTACTTCAGAGGCTGAGGTGGGAGGATCCCTTGAGCCCAG |
| | | AAGGTTGTGACTGCAGTGAGCCGTGA |
| 531 | patch | GGTGGGAGGATCCCTTGAGCCCAGAAGGTTGTGACTGCAGTGAGCC |
| | | GTGATTGTGCCACTCCACTCCAGCCTATGCAGCAGAGCAAGACCCTA |
| | | TCTCTAAAAAATAATTTAAAATAAATG |
| 532 | patch | CTATGCAGCAGAGCAAGACCCTATCTCTAAAAAATAATTTAAAATAAA |
| | | TGGCTGGGCCGGCCGCGGTGGCTCATGCCTGTAATCCCAGCACTTT |
| | | GGGAGGCTGAGGTGGGCGGATCACCT |
| 533 | patch | TCATGCCTGTAATCCCAGCACTTTGGGAGGCTGAGGTGGGCGGATC |
| | | ACCTGAGGTCCAGAGTTTGAGACTAGCCTGACCAACGTGGAGAAACT |
| | | CCATCTCTACTAAATACACAAAATTAG |
| 534 | patch | AGCCTGACCAACGTGGAGAAACTCCATCTCTACTAAATACACAAAATT |
| | | AGCCGGGTGTGGTGGCGCATGCCTGTAATCCCAGCTACTTGGGAGG |
| | | CTGAGGCAGGAGAATCACTTGAGCCT |
| 535 | patch | CTGTAATCCCAGCTACTTGGGAGGCTGAGGCAGGAGAATCACTTGAG |
| | | CCTGGGAGGTAGAGTTTGCAGTTGAGCCGAGATCACGGCACTGCAC |
| | | TCCAGCCTGGGCAACAAGAGCAAAACT |
| 536 | patch | GAGCCGAGATCACGGCACTGCACTCCAGCCTGGGCAACAAGAGCAA |
| | | AACTCCATCTCAAAATAATAATAATAAATAAATAAATTGGCTAAAATGT |
| | | TCAATTTTATGTTGTGTGTATGTTG |
| 537 | patch | TAAATAAATAAATTGGCTAAAATGTTCAATTTTATGTTGTGTGTATGTT |
| | | GCAGGTTTGTTTTTTTGAGAAGGCAAAGTCAGGCGTGGTGGCTCACA |
| | | CCTGTAATCCCAGCACTTTGGGAG |
| 538 | patch | GGCAAAGTCAGGCGTGGTGGCTCACACCTGTAATCCCAGCACTTTG |
| | | GGAGGCTGAGGTGGGCGGATCACAAGGTCAGGAGACTGAGACAATC |
| | | CTGGCCAACGCTGTGAAACCCTGTCTCT |
| 539 | patch | AGGTCAGGAGACTGAGACAATCCTGGCCAACGCTGTGAAACCCTGT |
| | | CTCTACCAAAAATACAAAAAATTAGCTGGGCGTGGTGGCATGTACCT |
| | | GTAATCCCAGCTACTCGGGAGGCTGAG |
| 540 | patch | GCTGGGCGTGGTGGCATGTACCTGTAATCCCAGCTACTCGGGAGGC |
| | | TGAGGCAGGAAAATCACTTGAACCAGGGAGTTGGAGGTTGCAGTAA |
| | | GCCTAGATCGCGCCACAGCACTCCAGCC |
| 541 | patch | TCACTTGAACCAGGGAGTTGGAGGTTGCAGTAAGCCTAGATCGCGC |
| | | CACAGCACTCCAGCCTGGTGACAGAGCGAGACTTCATATAAAAAAAA |
| | | AAAAGAGAGAGAGAGAGAAGGCAAAAA |
| 542 | patch | TACTAAAAATACAAAAAAATTAGCTGGGCATGGTGGCGGGCGCCTGT |
| | | AGTCCCAGCTACTTGGGAGGCTGAGGCAGAAGAATGGCTTGAACCC |
| | | GGGAGGTGGAGCTTGCAGTGAGCTGAG |
| 543 | patch | AGGCAGAAGAATGGCTTGAACCCGGGAGGTGGAGCTTGCAGTGAGC |
| | | TGAGATTGCGCCACTGCACTCCAGCCTGAGCGACAGAGCGAGACTC |
| | | CATCTCAAAACAAAACAAACAAACAAAA |
| 544 | patch | CTGCACTCCAGCCTGAGCGACAGAGCGAGACTCCATCTCAAAACAAA |
| | | ACAAACAAACAAAACAAAACAAAACAAAACAAAACAAAAAAAGATTCC |
| | | TTTGGCTACCTGGGTGGGTAGCCCA |
| 545 | patch | CTTCACAAGATGCAATGAAGAAGTGGGCTTTTTTTTTTTTTGAGACAG |
| | | AGTTTCTCTCTTGTCACCCAGGCTGGAGTGCCATGGCGCAATCTCAG |
| | | CTCACTGCAACCTCCACTTCCCGGG |
| 546 | patch | CTGGAGTGCCATGGCGCAATCTCAGCTCACTGCAACCTCCACTTCCC |
| | | GGGTTCAAGCGACTCTCCTGCCTCAGCTACCTGGGTGACTGGGATTA |
| | | CAGGCTTGCGCCACCATGCCCGGCTA |
| 547 | patch | CTGAGGTCAGGAGTTTGAGACCAGCCTGACCAACATGGAGAAACCC |
| | | CGTCTCTACTAAAAATACATAAATTAGCCGGGCATGGTGGCGCAAGC |
| | | CTGTAATCCCAGTCACCCAGGTAGCTG |
| 548 | patch | ACTTCTGGCCGGGTGTGGTGGCTCATGCCTGTAATCCCAGCACTTTG |
| | | GGAGGCTGAGGCGGGTGGATCACCTGAGGTCAGGAGTTTGAGACCA |
| | | GCCTGACCAACATGGAGAAACCCCGTC |
| 549 | patch | TCCCAAAGTGCTGGGATTACAGGCATGAGCCACCACACCCGGCCAG |
| | | AAGTAGGCCTTTACCCACTACATGCCTTCTTTTACTTATCTGCACAAT |
| | | GAAGATTAAAAATAGTTTCTTGGCCT |
| 550 | patch | CCTTCTTTTACTTATCTGCACAATGAAGATTAAAAATAGTTTCTTGGCC |
| | | TGGCATGGTGCCTCACACCTGTAATTCCAGCACTTTGGGAGGCTGAG |
| | | GTGGGCAGATCATTTGAGGTCAGA |
| 551 | patch | TAATTCCAGCACTTTGGGAGGCTGAGGTGGGGAGATCATTTGAGGTC |
| | | AGAAGTTCAAGACCAGTGTGGCCAACGTGGCGAAACCCCATCTCTAC |
| | | TAAAAATACAAAAAGTAGCCAGGCAT |
| 552 | patch | AACGTGGCGAAACCCCATCTCTACTAAAAATACAAAAAGTAGCCAGG |
| | | CATGGTGGCACGTGCCTGTAGTCCCAGACACTCAGGAGACTGAGGC |
| | | ACAAGAATCTCTTGAACCTGGGAGATG |
| 553 | patch | TTTGAGACAGAGTCTTGCTCTGTCCCCCAGGCTGGAGTGCAATGGCT |
| | | CGATGTCAGCTCACTGCAACCTCCATCTCCCAGGTTCAAGAGATTCT |
| | | TGTGCCTCAGTCTCCTGAGTGTCTGG |
| 554 | patch | GTATCAATTAAACATGGAGCAATTTAATTCTCACAGTAACCCTATGATA |
| | | AGAGACTTTTTTTTTTTTTTTTTTGAGACAGAGTCTTGCTCTGTCCCCC |
| | | AGGCTGGAGTGCAATGGCTCGA |
| 555 | patch | TTATCATAGGGTTACTGTGAGAATTAAATTGCTCCATGTTTAATTGATA |
| | | CATGATAAACTGATACTTAGCACAGGGCCTAGTAAATATCAGTGCTCT |
| | | AGTAGTGCCTGCCCATATTATTA |
| 556 | patch | ACAGGGCCTAGTAAATATCAGTGCTCTAGTAGTGCCTGCCCATATTAT |
| | | TATCATTGTTTGTGTCATTTATCTCTGTAGTAGGAAGTATATACAATCC |
| | | TATTGCATAGGTTTAAGGGAATT |
| 557 | patch | CTCTGTAGTAGGAAGTATATACAATCCTATTGCATAGGTTTAAGGGAA |
| | | TTAATCCACTAAAGTGTTTGGAACAGAGCCTGGCACATATTGCTATGT |
| | | AACTGCATCATCATCATCATCCCT |
| 558 | patch | ACAGAGCCTGGCACATATTGCTATGTAACTGCATCATCATCATCATCC |
| | | CTTTTTAATAGATGAGAAAACTGAATTTCAGAGAGGGTAGTCTGATCT |
| | | GAAATTCACCTATTATTCCAGCTT |
| 559 | patch | AACTGCATCATCATCATCATCCCTTTTTAATAGATGAGAAAACTGAATT |
| | | TCAGAGAGGGTAGTCTGATCTGAAATTCACCTATTATTCCAGCTTCTC |
| | | TCTTCCAAATACGGCCTCTGTGC |
| 560 | patch | AGAATTCTCATTCAGAACCTTGATCACAGGCCGGGCGCGCCGGGATT |
| | | GCTCACGCCTGTAATCCCAACACTTTGGGAGGCCGAGGTGGGAGGA |
| | | TCACTTGAGCCCAGGATTTGGAGACTA |
| 561 | patch | TTTGGGAGGCCGAGGTGGGAGGATCACTTGAGCCCAGGATTTGGAG |
| | | ACTAGCCTGGGCAACATAGCGAGACCCCTTCTCTAAAAAAGCAAACA |
| | | GGCAAAACTTCATGAGAATCTTGATCA |
| 562 | patch | CCCCTTCTCTAAAAAAGCAAACAGGCAAAACTTCATGAGAATCTTGAT |
| | | CATGTTAAAATTTTATGTCCTTCGATTTCTCCCTACACACACACACACA |
| | | CACACACACACACACACACACAC |
| 563 | patch | TGAGAATCTTGATCATGTTAAAATTTTATGTCCTTCGATTTCTCCCTAC |
| | | ACACACACACACACACACACACACACACACACACACACACACTCAAC |
| | | ATTTCCTCCACCCATATCATCACT |
| 564 | patch | TAGTTTTATCCTGTTTTCCGAAAAACAATCATTTATTTATTTATTTATTT |
| | | ATTTAATTTTATGAGACAGGGTCTGGCTTTGTCACCCAGGCTGGAGT |
| | | GCAGTGGTGCGATCTTGGCTCAC |
| 565 | patch | GTCTGGCTTTGTCACCCAGGCTGGAGTGCAGTGGTGCGATCTTGGC |
| | | TCACTGCAACCTCTGCCTCTCAGATTCAAGCCATCCTTCCACCTCAG |
| | | CTCTGCCACTGAGTAGCTGAGACTACA |
| 566 | patch | TTCAAGCCATCCTTCCACCTCAGCTCTGCCACTGAGTAGCTGAGACT |
| | | ACAAGCACTCGCCACCATGCCCGGCTAATTAAAAAAATAATAATCATT |
| | | TTAAATGCAAGCTTTATATTATAAA |
| 567 | patch | GGTAATTAAAAAAATAATAATGATTTTAAATGGAAGGTTTATATTATAAA |
| | | TACAAAGTAAACATGAAAATAAAACCCAAACATAGCAGTGTTATTAAA |
| | | CTCTGGCCTGTAGCAGTGGCTC |
| 568 | patch | AAAACCCAAACATAGCAGTGTTATTAAACTCTGGCCTGTAGCAGTGG |
| | | CTCACACCTGTAATCCTAGCAGTTTGGAGGCCGAGACAGGTGGATTA |
| | | CTTGAGACCTGGAGTTTGAGACCAGC |
| 569 | patch | TTGGAGGCCGAGACAGGTGGATTACTTGAGACCTGGAGTTTGAGAC |
| | | CAGCCCAGGTGACACAGCAAGACCTCATCTCTACTAAAAATAAAAAAA |
| | | AATTAGCCAGGTGTGGTGGTATGCAC |
| 570 | patch | TCATCTCTACTAAAAATAAAAAAAAATTAGCCAGGTGTGGTGGTATGC |
| | | ACCTGTGGTCCCAGCTACTTAGGATGCTGGAGTGCGAGGATCGCTT |
| | | GAGCCCAGGAGGTCAAGGCTGCAGTG |
| 571 | patch | GATGCTGGAGTGCGAGGATCGCTTGAGCCCAGGAGGTCAAGGCTGC |
| | | AGTGAACTATGATCACTCATTACACCCCAGCCTGGGTGACAGAGCGA |
| | | GATGCTGTCTCAAAACAAAACAAAACG |
| 572 | patch | CCCCAGCCTGGGTGACAGAGCGAGATGCTGTCTCAAAACAAAACAAA |
| | | ACGAAAAACAACTCTGGCTAGATGCTATTGCTTGCCAAGGGTGCAGT |
| | | CTTCCATTTATTAAAAGTGAAAATTA |
| 573 | patch | GCTATTGCTTGCCAAGGGTGCAGTCTTCCATTTATTAAAAGTGAAAAT |
| | | TAGGGCCAGGCACATTGGCTCATGCCTGTAATCCCAGCACTTTGGGA |
| | | GGCTGAGGTGGGTGGATCACCTGAG |
| 574 | patch | TGCCTGTAATCCCAGCACTTTGGGAGGCTGAGGTGGGTGGATCACC |
| | | TGAGGTCAGGAGTTCGAGACCAGCCTGGCCAACATGGTGAAACCTTA |
| | | TCTCTGCCAAAAATATAAAAGATTAGC |
| 575 | patch | CTGGCCAACATGGTGAAACCTTATCTCTGCCAAAAATATAAAAGATTA |
| | | GCCATGTGTCGTGGTGGGTGCTTGTAATCTCAGCTACTTGGGAGGCT |
| | | GAGGCAGGAGAATCACTTGAACCCA |
| 576 | patch | TGTAATCTCAGCTACTTGGGAGGCTGAGGCAGGAGAATCACTTGAAC |
| | | CCAGGAGGCAGAGGTTGCAGTGAGCCAAGATTGTGCCATTGCACTC |
| | | CAGCCTGTGCAACGAGCGAAACTCCAA |
| 577 | patch | TCAGCTACTTGGGAGGCTGAGGCAGGAGAATCACTTGAACCCAGGA |
| | | GGCAGAGGTTGCAGTGAGCCAAGATTGTGCCATTGCACTCCAGCCT |
| | | GTGCAACGAGCGAAACTCCAACTCAAAA |

### APPLICATION

Profiling of ctDNA may offer a non-invasive approach to estimate disease burden and monitor disease progression. Embodiments of the method described herein provide a quantitative method, which exploits local tissue-specific and gene-specific cfDNA degradation patterns, that can accurately estimate ctDNA burden independent of genomic aberrations.

Nucleosome-dependent cfDNA degradation at selected NDRs (e.g. promoters and first exon-intron junctions) is shown herein to be strongly associated with differential transcriptional activity in tumors and blood. A machine learning model that was developed based on expression-specific DNA degradation patterns was found to be capable of accurately predicting ctDNA fractions (see examples). Leveraging on these findings, embodiments of the methods enable for the first time the detection of tumor DNA burden (even of very low frequency) in blood by only sequencing selected NDRs in cfDNA assays. From only less than 50 kb DNA sequence in total (4 kb×6 features or 4 kb×10 features), embodiments of the methods can accurately predict ctDNA levels, and thereby monitor the dynamics of the systemic tumor burden over time from blood/liquid samples. Indeed, using compact targeted sequencing (<25 kb) of predictive regions, the disclosure demonstrates how embodiments of the method enable quantitative low-cost tracking of ctDNA dynamics and disease progression.

Embodiments of the method enjoy several advantages including cost efficiency, flexibility, high accuracy and high sensitivity.

Embodiments of the method requires less sequencing and are therefore cost-efficient. In embodiments of the method, 100× less DNA sequencing (e.g. ˜30 kb at 100× coverage) is needed than low-pass WGS-based methods requiring whole genome sequencing at ˜0.1×. The sequencing cost is also comparable to sequencing a panel at 10,000× (usual target for coding mutation panels). Embodiments of the method also require less sequencing than standard targeted sequencing assays, which usually require more than 1000 kb DNA sequence.

Further, embodiments of the method can be implemented as an extension/add-on to a standard targeted panel assay, providing flexibility and further allowing for an extremely cost-effective approach to generic ctDNA profiling. For example, the NDRs identified herein can be easily added to existing cfDNA capture panels, eliminating the need to perform two separate assays. Notably, WGS or methylation-based assays do not enjoy this flexibility.

Last but not least, embodiments of the method are capable of accurately estimating cancer cell-free DNA burden with a mean deviation of about 3.4%. As compared to conventional coding panel that usually fail (no mutations) in more than 20-30% of patients, embodiments of the method are shown to be able to accurately predict cancer cfDNA in most cancer patients. As demonstrated in the examples, both colorectal cancer and pan-cancer models have high prediction accuracy, with the pan-cancer model generalizing well to most/all solid tumor types.

Overall, embodiments of the method enable quantitative low-cost tracking of ctDNA dynamics and disease progression, and would be invaluable in the clinical setting.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of estimating a circulating tumor DNA (ctDNA) burden in a subject, the method comprising:
determining in a blood sample obtained from the subject, a level of cell-free DNA (cfDNA) that maps to one or more nucleosome-depleted region (NDR); and
estimating the ctDNA burden based on said level of cfDNA,
wherein said NDR
(i) comprises the NDR of a gene which transcript is differentially expressed between healthy blood sample and tumor sample and/or
(ii) is degraded to different extents between cfDNA in healthy blood sample and cfDNA in blood sample of a tumor-bearing subject.

2. The method according to claim 1, wherein determining in a blood sample obtained from the subject, a level of cfDNA that maps to one or more NDR comprises:
sequencing cfDNA fragments in the blood sample to obtain sequencing reads; and
determining the number of sequencing reads that align with the one or more NDR to obtain said level of cfDNA that maps to one or more NDR.

3. The method according to claim 2, further comprising
contacting the blood sample with one or more probe capable of binding to the one or more NDR to capture cfDNA fragments comprising the one or more NDR prior to the sequencing.

4. The method according to claim 1, wherein the NDR is selected from the group consisting of: a promoter region, a first exon-intron junction and combinations thereof.

5. The method according to claim 1, wherein the estimated ctDNA burden is positively associated with a tumor burden in the subject.

6. The method according to claim 1, wherein said transcript that is differentially expressed between healthy blood sample and tumor sample comprises a transcript which FPKM (Fragments Per Kilobase of transcript per Million), or RPKM (Reads Per Kilobase Million), or TPM (Transcripts Per Kilobase Million) value differs between healthy blood sample and tumor sample, optionally wherein the value differs by at least 10 times between healthy sample and tumor sample.

7. The method according to claim 1, wherein said NDR that is degraded to different extents between cfDNA in healthy blood sample and cfDNA in blood sample of a tumor-bearing subject comprises a NDR having different sequencing coverage in healthy blood sample and in tumor sample.

8. The method according to claim 1, wherein said transcript that is differentially expressed in healthy blood sample and tumor sample is selected from the group consisting of: a transcript that is more highly expressed in healthy blood sample than in tumor sample, a transcript that is more highly expressed in tumor sample than in healthy blood sample and combinations thereof.

9. The method according to claim 1, wherein said transcript which is differentially expressed between blood sample and tumor sample consists of transcript(s) that is more highly expressed in blood sample than in tumor sample.

10. The method according to claim 1, wherein the one or more NDR comprises at least two NDRs, optionally six NDRs, further optionally ten NDRs.

11. The method according to claim 1, wherein the total length of the one or more NDR is no more than 30 kb.

12. The method according to claim 1, wherein the one or more NDR comprises one or more NDR of a gene selected from the group consisting of: SLC11A1, NLRP12, PRTN3, HMBS, LILRB3, ACSL1, GP9, MX2, RASGRP4, ATG16L2 and combinations thereof.

13. The method according to claim 1, wherein the method is a method of determining disease progression in a subject and the method further comprises:
determining in a subsequent blood sample obtained from the subject, a level of cfDNA that maps to one or more NDR;
estimating the ctDNA burden based on said level of cfDNA;
comparing the ctDNA burden estimated from said subsequent blood sample with the ctDNA burden estimated from said blood sample; and
identifying the subject as having disease progression if the ctDNA burden estimated from said subsequent blood sample is higher than the ctDNA burden estimated from said blood sample and identifying otherwise if the ctDNA burden estimated from said subsequent blood sample is not higher than the ctDNA burden estimated from said blood sample.

14. The method according to claim 13, the method further comprising changing the treatment regimen received by the subject if the subject is identified as having disease progression.

15. The method according to claim 1, wherein the tumor comprises colorectal tumor.

16. The method according to claim 15, wherein the one or more NDR comprises one or more NDR of a gene selected from the group consisting of: SHKBP1, ACSL1, BCAR1, RAB25, PRTN3, LSR and combinations thereof.

17. A kit for estimating a ctDNA burden in a subject, the kit comprising one or more probe that is capable of binding to one or more NDR, wherein said NDR
(i) comprises the NDR of a gene which transcript is differentially expressed between healthy blood sample and tumor sample and/or
(ii) is degraded to different extents between cfDNA in healthy blood sample and cfDNA in blood sample of a tumor-bearing subject.

18. The kit according to claim 17, wherein said one or more NDR comprises one or more NDR of a gene selected from the group consisting of: SLC11A1, NLRP12, PRTN3, HMBS, LILRB3, ACSL1, GP9, MX2, RASGRP4, ATG16L2 and combinations thereof.

19. The kit according to claim 17, wherein said tumor comprises colorectal tumor and wherein said one or more NDR comprises one or more NDR of a gene selected from the group consisting of: SHKBP1, ACSL1, BCAR1, RAB25, PRTN3, LSR and combinations thereof.

20. The kit according to claim 19, wherein the one or more probe comprises the sequence of one or more of SEQ ID NO: 1 to SEQ ID NO: 577, or a sequence sharing at least 75% sequence identity thereto.


SEQUENCE LISTING (577 sequences)

SEQ ID NO: 1 - DNA, 120 bp, Artificial Sequence
  degradation probe
tggcattagggaccccaaatccaggtctgctcccagtgccataggaagcaaccagcccccaacccccacctgctcttgggagctggcccacaccagaaatggactgagtagacatgagaa

SEQ ID NO: 2 - DNA, 120 bp, Artificial Sequence
  degradation probe
ctgaccccagcttccaccaatgccgttaagatgccgccacttgggtgaggggctcctccaggtactgcaccaaagcctgggcctgcagagggagaacagagatcagtcccgcacacagcc

SEQ ID NO: 3 - DNA, 120 bp, Artificial Sequence
  degradation probe
ccaagacacatgacaggagttgaagtttattcttggaaaaaacaaagtcccatcctccccccattgtctaagaaggttcttctaggaggccccgcccctccaaatggtcatttctctttt

SEQ ID NO: 4 - DNA, 120 bp, Artificial Sequence
  degradation probe
ctactttcttggatgggaggaatcagagaagataggaagaagcaggcatcgcttctaggattcctcatgttctgcaaggatggacttctacccaccccttcaccatgaccttctgcgaac

SEQ ID NO: 5 - DNA, 120 bp, Artificial Sequence
  degradation probe
aatggttcttggcagttacgccaaatagtccttgtcctcaccaatggcatgacctcacctagaccctgctgctcagaacagagtcttacccacctcataggcaaaactgggagtgagctc

SEQ ID NO: 6 - DNA, 119 bp, Artificial Sequence
  degradation probe
agaccctctcaaaaaaataaaaggcacgcacgcccccaaacctgaaatgatagacactaacctctcagggcctttatattctctgtgcaacacaacccccaccagacccttgcacctct

SEQ ID NO: 7 - DNA, 120 bp, Artificial Sequence
  degradation probe
tgtgtaagcacataaacacctggaagaagaagaaagggatagcttgttctaggctgttaggggcaccctcccaaccccaagaatagaaaaggcactcactggccagtcgaagtggttcat

SEQ ID NO: 8 - DNA, 120 bp, Artificial Sequence
  degradation probe
gtgtttatgtgcttacacagaccctccccagatcttgaagccctttgtagacaagaggcctttgctgtgaggcctcaggtcctgtacagggagtagaagtaacccagacctcccaaaggc

SEQ ID NO: 9 - DNA, 120 bp, Artificial Sequence
  degradation probe
acccagacctcccaaaggccttcatctccagaccctgctcctacactcagaccactcaccccagctgtctgtttctgccccaggtctctgcctacccaatatccttaaagcaggtctgta

SEQ ID NO: 10 - DNA, 120 bp, Artificial Sequence
  degradation probe
tccttaaagcaggtctgtatagggagctaccagcccccttccccagtgccatataaaggaacccatcccagagaagtgggtaggtttcgagtcccagctccacccaagatgagaaacact

SEQ ID NO: 11 - DNA, 120 bp, Artificial Sequence
  degradation probe
acccaagatgagaaacactgcccatcccaggccctgggctgtcaaggaagggcagaagttactgtggccatgatactcccacagctctgcattgcagcagggtgactggtgttctatcag

SEQ ID NO: 12 - DNA, 120 bp, Artificial Sequence
  degradation probe
gtgactggtgttctatcagcccctggtctaatatcccttactctggttcccccagtgtacccccctaccagctctgaaggaaaagcaggccaagtgggtttcttcctttttttttttttt

SEQ ID NO: 13 - DNA, 120 bp, Artificial Sequence
  degradation probe
cactgagggctgagtgtgcattgctgggcttctggaatctgtggtgagggccggtcccccaactaatgagcaagaaatagcccctcagaaacccacttgaggccaggcgcggtggctcac

SEQ ID NO: 14 - DNA, 120 bp, Artificial Sequence
  degradation probe
gtgggctgtctctgaaggtcctgtccctttttcgcttcccccccgctggagctgcttctcccgcttgcgggagcccaggctgagagcagacacccaacctgtcgaacctgtctgacgtca

SEQ ID NO: 15 - DNA, 120 bp, Artificial Sequence
  degradation probe
tcatcatctctccacccacctgggccccaggtctccagccaccccgctcttcctgttctcagcttccgtcctctctgcttccttacagcacccccacctgccagagctgatcctccctag

SEQ ID NO: 16 - DNA, 120 bp, Artificial Sequence
  degradation probe
gctcaatgccctcaaccctcagctctcaaaagatctggtcctgcccctcctctcattgggggtaaggggacttctgcagccagagggattgggggccaactcaaggttaggcagggccta

SEQ ID NO: 17 - DNA, 120 bp, Artificial Sequence
  degradation probe
gaacccaactcacccttgaagacaaagttataatcttcctcagttccattccccatcttggctccgcatggagggtgcaggtgtcttcggggacagaggcgacaaatctgtgtgttggct

SEQ ID NO: 18 - DNA, 120 bp, Artificial Sequence
  degradation probe
ttctgaaggaatgaggagccccaggaggtcttggtaagagataaaaaaacaggagggggccctctctggctctattcaatctctttctgggtctctcaggcttcctcttgcttcagggaa

SEQ ID NO: 19 - DNA, 120 bp, Artificial Sequence
  degradation probe
taactcagcactcttaaggaggaatcaacatcattatttaccacagtagagcatactcagaagctccctgacttttgcccctgcaaccagaccctcccccactccctgcagctttctttc

SEQ ID NO: 20 - DNA, 119 bp, Artificial Sequence
  degradation probe
ttaggctcctctgccccgttcctaatagaaacccaaatccctggtgtcagccagtgcagcacaagagatggaggtcagacttcagaaaggacttccctcttttatttttttttctgaga

SEQ ID NO: 21 - DNA, 120 bp, Artificial Sequence
  degradation probe
atgagccaccacacccggccaggacttctctcttgagaagaaagggaaaccagcaaacaagaggaagaaagtcatcttttctgggcaggtgtacagggaacgagagaaaggagagacaca

SEQ ID NO: 22 - DNA, 120 bp, Artificial Sequence
  degradation probe
cagacgaggctacaaccctaggagtcctctggattctatcagactctgcagcaaattcatgatattttctgtcttccctatgtatttattgccccaccctataatgggatgtgtctctcc

SEQ ID NO: 23 - DNA, 120 bp, Artificial Sequence
  degradation probe
agcctcgtctgcctggcccttggctgactcactggacctgcataccaggcactgggctcaactttgctgtccctggaagcatgcatgtatctttctagcccctgtgtttggtgcctttgc

SEQ ID NO: 24 - DNA, 120 bp, Artificial Sequence
  degradation probe
ggtgcctttgctgttttccaaccccaccttcatacttcctttagggcagggtggactctccagccaccctaccccaatgcctctcccaacctcagcccggcccctcccttgagctgggac

SEQ ID NO: 25 - DNA, 120 bp, Artificial Sequence
  degradation probe
aatggtagttcctcatattattgccccagactattgctaaccctttccttcctggaaaagatgccagtagtacaggttccccagctcaagggcgggttctggagtgagagtcccagctca

SEQ ID NO: 26 - DNA, 120 bp, Artificial Sequence
  degradation probe
tcccctactgggcacatctccctcatccctctcagtgggtgggctctcagccctagcgtcataccacaaaggtgcttagggcaagggaattgaagtcagactttgtggccttttgtaaac

SEQ ID NO: 27 - DNA, 120 bp, Artificial Sequence
  degradation probe
gtgtgtatatgtgtgtgtgtgttcctgcctctcccctactgggcacatctccctcatccctctcagtgggtgggctctcagccctagcgtcataccacaaaggtgcttagggcaagggaa

SEQ ID NO: 28 - DNA, 120 bp, Artificial Sequence
  degradation probe
cacacacgcatacacacacatcctcttacccacacatatccatattatttccagcttttccatactgtcctatgcaaataaggaatgtttaaatatatattttttatttttattttttga

SEQ ID NO: 29 - DNA, 120 bp, Artificial Sequence
  degradation probe
ccaggaccaagtggggtgatcaggacccctccaggtgggtcccgggtgcggatggccagttgggcgcatgcttattgggcctgcatccgttggccacggaggctctttgggacaagcaaa

SEQ ID NO: 30 - DNA, 120 bp, Artificial Sequence
  degradation probe
ggccaccctgttgttgtgggagtattaggggaagttgccactaaggctggcaggtcctggagttccacccaggatggggacatgggggagggaagaggaggtgcccctcagagcctgagg

SEQ ID NO: 31 - DNA, 120 bp, Artificial Sequence
  degradation probe
ggataggagccatgctcccctggggtggctctggaacgtgggccccgtgcacaggctatgtagtctgctcacacccttgctgcctgccacccgggcacccaggacttgagcccagaccct

SEQ ID NO: 32 - DNA, 120 bp, Artificial Sequence
  degradation probe
gccaccccacaggtggctttttattccctcctgctgctgagtgtcacgcgtggggctgagggcctgcacgccttcccggcctcatcctctccgacgattcacgccctcctaaggctggca

SEQ ID NO: 33 - DNA, 120 bp, Artificial Sequence
  degradation probe
cactgccaagggctccccgacacaagcacagagcacagcctggccttggctgcagcaccacagcagatgcctttatgcccagggccagctatgggctgaaagcacaccccagtcctcact

SEQ ID NO: 34 - DNA, 120 bp, Artificial Sequence
  degradation probe
tccgcactgtctgtggggtgttttgggttgcaggttccatgtcctgaagcaggagagccctccccatgccacctcctctgcccagcctcttagggacagtgcctgggatgccctggctgg

SEQ ID NO: 35 - DNA, 120 bp, Artificial Sequence
  degradation probe
cagcctcacaccgagctctctgccaaaaccccggatcacaagcatgagctccagcaggtccgcttgagtgaacaccgctgatcctgcctgcaggggtataggcctggggcctgccagccg

SEQ ID NO: 36 - DNA, 120 bp, Artificial Sequence
  degradation probe
ttctagcctggggcagaagtagggtggaggggtgggtctagcctccttgatgacacagttggggttggaggtgggggctacccctattctgggcctggtgctgtccctgaagtcaggggt

SEQ ID NO: 37 - DNA, 120 bp, Artificial Sequence
  degradation probe
tgtagaggtgggaggaagtcagggaagcaagacacaggtggagccagggcctctcctgtgctcggagggccaggaagggcctgacagagggcagagccccaccctcttacacggagacca

SEQ ID NO: 38 - DNA, 120 bp, Artificial Sequence
  degradation probe
aggaggcagttttggagtcagaggtgaagtgtggggagtgggattgggtgatagggaagtttctccagcaaagacactgggcaagcctcttccttggcccccagataaagggctgcaggg

SEQ ID NO: 39 - DNA, 120 bp, Artificial Sequence
  degradation probe
cccttttcttctgagcctggacaactccttcccttgcccccaggagcccacagccggctaaaccaactggtcagctagaaccaaccagcaggggaggtgcgctccaggggggcttctaag

SEQ ID NO: 40 - DNA, 120 bp, Artificial Sequence
  degradation probe
tggtaggacctcctaaagggacagcagatgccaggagagggccgcaaaggcacagcgttggaaccagagggttcctaccaagttgacaactccaaaagagcatggtggtcccccaaggac

SEQ ID NO: 41 - DNA, 119 bp, Artificial Sequence
  degradation probe
acagagagacagtcagttgtcctcagaattggatccagaacctcgtcttctggcatggccccccagtgcagcaccacatgtggcctccgaattcccagtctgggattgggggagctctg

SEQ ID NO: 42 - DNA, 120 bp, Artificial Sequence
  degradation probe
tggccccaggtctaggcgagggatggaaataaggacagtgccactagggcctcctgtctgtacaggaggggccctgagacagctcctcaaacccctttcccattatacggatggtgcaga

SEQ ID NO: 43 - DNA, 120 bp, Artificial Sequence
  degradation probe
ggccatggtaccagtacacagtgctcaccctcctgctcagacaaacagtaacggccagaagctccaagcctcctgccccagcccaaggtgactttcctggcagaacctgggactgccctg

SEQ ID NO: 44 - DNA, 120 bp, Artificial Sequence
  degradation probe
ccctgccctgccctgcctccacccagctcccgggcaagcagtgccaggaggaagtgtcagcagctcctccccaaagctcttttccccacagccccgccacccccgtacctgggccccgat

SEQ ID NO: 45 - DNA, 120 bp, Artificial Sequence
  degradation probe
cccatcgccctgggccgggacacgccccctcttgtgcccattcctttccctcggtggagagttgggtggtgggggctggccggcctgtgagagcaggtgtcggggtgcctggagaatcgg

SEQ ID NO: 46 - DNA, 120 bp, Artificial Sequence
  degradation probe
tccctcctgagagtccctggtttcctttcccacacccctcccggggcccgccgcagctttggtcaccttctcagaatgaccatgggttgatctcttctttgctagtgagatgtttcccat

SEQ ID NO: 47 - DNA, 120 bp, Artificial Sequence
  degradation probe
agggaacgggagccttgtggtagaaaaggagctgctgtccccgatctggaactcgaaaccccaacatcttccaggccgtaagcccctcaccccgcacccctaggcaaatctccgctttcc

SEQ ID NO: 48 - DNA, 120 bp, Artificial Sequence
  degradation probe
tttcccaactccctcacctcggtccagcgatcagcctctcggaccctcgagagggagctccgaccctgctctagctcttgcgggtcccccaaccatggactccaaagccttcatgtccac

SEQ ID NO: 49 - DNA, 120 bp, Artificial Sequence
  degradation probe
cgccgcgggctttgcagccccgcacgccgagcgccggtggaagccgggtcctggccacaggagtcctagccgcacgcgagtgtcgcgggagggggccagggccgggttctgcagtgtgga

SEQ ID NO: 50 - DNA, 120 bp, Artificial Sequence
  degradation probe
cggcgcgcccgcccccactcccgctccgctcccggaaccccgcgccggcggccggagtgaatcattaactggagccgggtgctccggctgcgcagccttccccgctggcgccacgcactt

SEQ ID NO: 51 - DNA, 117 bp, Artificial Sequence
  degradation probe
cacttgcccgcccggcctcggtcccccggagctcctccctctccgggagccggtgggcagtccccacgccacaccagccggaccacccgccagaggctcaacggcccctcctcgcag

SEQ ID NO: 52 - DNA, 120 bp, Artificial Sequence
  degradation probe
gcgttggcggggccgggcgcgggggccgccagccggtaggagggttcctgtgtcgggaaagtggctggcggggcccgcgctccccggccgacggccgcctgctgtgtcaccttgggcctc

SEQ ID NO: 53 - DNA, 120 bp, Artificial Sequence
  degradation probe
ggccccgccaacgcacccgaggcgcacaggctccggagcccccgcggttcctgcctcccggcttcccggccagggcagaaggagatcccagggccgcggaccccggccggcggttcccag

SEQ ID NO: 54 - DNA, 120 bp, Artificial Sequence
  degradation probe′
ccggcggttcccaggccccgcaggtccggcaggaaatgccgctcgcttaaccccttccagcccgctgccgcctccaagcccgtacgcggcccggccccacctggcgccctcaccaggtgg

SEQ ID NO: 55 - DNA, 120 bp, Artificial Sequence
  degradation probe
accggggcggctgcggcgcgctcggagccccgagggcacgcggcccgggcagctcggtgtgcgcccccgcgagagccggggccccaggcccgccggacaccatgaaccacctggtgaggg

SEQ ID NO: 56 - DNA, 120 bp, Artificial Sequence
  degradation probe
gcagccgccccggtgccgccgcgcagctgccgcctcggccacccagagccggtccagccgctctccgcctgccgccacggcccagccggcccagcccgatcccagcatgcccggccgcgc

SEQ ID NO: 57 - DNA, 120 bp, Artificial Sequence
  degradation probe
gggggcggggcggggggcggcccctactgggcgggcttctgtctgcgcgcccgcgcccccgacaggcggcttatttgcatggagagcggcggcggccaatgggcgcgcgcggccgggcat

SEQ ID NO: 58 - DNA, 120 bp, Artificial Sequence
  degradation probe
gaggctccaggcccctgcccgaggccaggcttccagcctagggcgcaggacaccgctctccaacaaatgcacgtccccgccttccacccaggcgagaacgcaccgaaggcctacgcaaac

SEQ ID NO: 59 - DNA, 120 bp, Artificial Sequence
  degradation probe
acgcaaactcctccacttctgggaaggctgcccggtttgccttcttcctccagagccgagacatggcctcaagtcgacttgtctttcccgctaaccaaccccagggcattcatcgcgtgt

SEQ ID NO: 60 - DNA, 120 bp, Artificial Sequence
  degradation probe
ggtgggaccgtctacagtcggagctgtggtggtgatgattcgtctcctacaggctggggcacgccccctcccctttccctctagattcagatttgagtcccacggcccatggacacgcga

SEQ ID NO: 61 - DNA, 120 bp, Artificial Sequence
  degradation probe
tccccagatgtcggcacaatgcaggggaaggtaggtgctgcccgtgaaaagcgctgggtcgagagctcgccagcctccctgcttgaggcttcatttcctggggtccttacacggtgggac

SEQ ID NO: 62 - DNA, 120 bp, Artificial Sequence
  degradation probe
gctgaagcagatgagttggagcaggactcaggagggtactaggcccaggttttaggctgaacctaggaatgagtaatgagaggaggcagatcagctggaggaccagcccgagtccccaga

SEQ ID NO: 63 - DNA, 120 bp, Artificial Sequence
  degradation probe
accgagcaaggggcaactcaggagagccaaggttgtctccactcggtttcccccgagtgattcttccttctatctccctcattcccccagtgccgctctcctaccccatgtcgctgaagc

SEQ ID NO: 64 - DNA, 120 bp, Artificial Sequence
  degradation probe
tgctcggttgcaggtcagtgactgtcaccccatgcagcagaaccagggtgccagagcccctgggccaggccagggaggcacaatctgcagggagatgataggaccgagcctggtccagac

SEQ ID NO: 65 - DNA, 120 bp, Artificial Sequence
  degradation probe
ctctgctgcgtgaagccgggtctaccccgctgcttccctgccaccagggctgcctcgtggcatcccattgctcagccagtggcccaggattgttgactgagggtcctgtctggtctggac

SEQ ID NO: 66 - DNA, 120 bp, Artificial Sequence
  degradation probe
cagcagagaattctcaagcatggcctatggaagacacaagcccaggtgggcctgcagctgcccctctgtctgacccccagggctcagcctacgcagcgcctggccagatgtgccaccttg

SEQ ID NO: 67 - DNA, 120 bp, Artificial Sequence
  degradation probe
agactcctgtgtcccacccctcccaaaaaactggacgtctgcatttcttcaatatttggcttgggaagcattggccagaactcacagcacagcgggtgaggaaggagcagtgggatgaga

SEQ ID NO: 68 - DNA, 120 bp, Artificial Sequence
  degradation probe
gacagagcatgacccagagtcctcaggaggaacagaacagctagccaacaccctgtgcctccccgacacagcccgcctcgcctcagaagaggagccagccttcagggaagctgcctggag

SEQ ID NO: 69 - DNA, 120 bp, Artificial Sequence
  degradation probe
agcatctcctcctgcttgttcccaagagctgtgccgggcccctcctgcaccctgaccctgtccagccacccactcctcagcctggctggcacaccagcttcccagagagcaggtctgaac

SEQ ID NO: 70 - DNA, 120 bp, Artificial Sequence
  degradation probe
gcagagactgctgcctggggccctgccagcagggcactgcctgcccatgaggcctggtctcccctccccacagaagtggaggcccagggatgacctcaggctcctgagggtcccgctggt

SEQ ID NO: 71 - DNA, 120 bp, Artificial Sequence
  degradation probe
tcatcccccaaggttccagccatccacctcctctctgcaccgccctccttggccaactaggatggagcaggggccagctcccttgagggccaggccttttgtgtccttgcccgtccctgc

SEQ ID NO: 72 - DNA, 120 bp, Artificial Sequence
  degradation probe
aaaaggagcaaatttttgaaatgcctgtgtcggagcctgtttcccttgctgcctcctgctcacaccagcagccccacccccagtgctgttctaatgcagctactgggggaggagggtctc

SEQ ID NO: 73 - DNA, 120 bp, Artificial Sequence
  degradation probe
ccccaccccccagctacatccccatcaacactgtggggcttaacttctggggccttgtttccaaagcggaaatgccattctgggaggcacaggcttttgggggagggaatttaagtggaa

SEQ ID NO: 74 - DNA, 120 bp, Artificial Sequence
  degradation probe
gggtatgtggggctgcatccctgtccttgacagcggaggtccgggaggctggactgggggtggggtgaggttctgcccccatataccccggccagagcctggccagtgggtgctcctccc

SEQ ID NO: 75 - DNA, 120 bp, Artificial Sequence
  degradation probe
tctcaaaaaaaaaaaaaattggggctaacggtgagcctgttttgcaggattgctgtgtgcacaaaataaatgcatgggcatagggcccctggcactggctgggcgcagaggctgtgctgg

SEQ ID NO: 76 - DNA, 120 bp, Artificial Sequence
  degradation probe
tttcttccctggccctgtcccttctgtgccccgaaatgtgtgggatggggcagtggttggtaaactgggtctcggtctcttcatgtgaaaaatggggctaggctgggtgcagtggctcac

SEQ ID NO: 77 - DNA, 120 bp, Artificial Sequence
  degradation probe
gcctgtccttttgcagtctgagtgtgacaggggtgtttggctttctgagcctggagtgctgctgtccctgtccctagattcccttcctgctgcagggtgggctgaggtctagcagcccca

SEQ ID NO: 78 - DNA, 120 bp, Artificial Sequence
  degradation probe
tcaacaaatgtttactgcaccccttctcaccaatggggctaaacttagtggggtgggggtggtgctggccccagtgctgagctcagttttactggggaggaaggtgcttaggtctcagtt

SEQ ID NO: 79 - DNA, 120 bp, Artificial Sequence
  degradation probe
gtgtgtgcctctgcacttaggcgtgacttaaccacccccatcctccagactggggcagcctcagatttcttcacccaggccagcctatcccatgagggccacctctgaggtcccacccag

SEQ ID NO: 80 - DNA, 120 bp, Artificial Sequence
  degradation probe
gaccattgaggagagctggtggtcactctgcagagcgtccctccgtggggatggtcctgggtggcctcaggactcaatggagggagtcccgggcgtggcgtgtgtctgtctcaggccccc

SEQ ID NO: 81 - DNA, 120 bp, Artificial Sequence
  degradation probe
gccccctgagtatcaccagccaccctttgctgggtcctgggtacacaagccacccatcttcagatgtcagggtctcctccagcgggcgcagtccctctgccctccctcagcccttgggac

SEQ ID NO: 82 - DNA, 120 bp, Artificial Sequence
  degradation probe
tccctcccccggcctccctcccccagcttccctaaattctgtcctcttgctcaaccctccaggctgtgatcgaagccagggcgcgaaagttgattccaaatggccccctctgccccagcc

SEQ ID NO: 83 - DNA, 120 bp, Artificial Sequence
  degradation probe
agagaagcactgaatgaacttggaggccaaaaagaaaaaaagctttttaacgtttcatttggaaaggatttcacgcttacagaagtccaaagggtccaaactccctccccgctccccaaa

SEQ ID NO: 84 - DNA, 120 bp, Artificial Sequence
  degradation probe
ttttctttttggcctccaagttcattcagtgcttctctccatttcactgtaggtttaaaccgttcagttataaataaataattttatcctctgtcctcggctttcccccgtttccggcac

SEQ ID NO: 85 - DNA, 120 bp, Artificial Sequence
  degradation probe
gacttggagggagctcctggcagggccctgcagtgcccaggatggccccgccgcagagacaaatccagcccccatgtctgcagtgccggaaacgggggaaagccgaggacagaggataaa

SEQ ID NO: 86 - DNA, 120 bp, Artificial Sequence
  degradation probe
agaatcaccccagggttagaggattccacagaccccaagagactctgagtcccatgatcctctgccctgctgaggtcccaggaggctgcacacattgaaatccacaccctgagtgggagg

SEQ ID NO: 87 - DNA, 120 bp, Artificial Sequence
  degradation probe
tcccgatggggcgtacggcataaacccgggggaccccagctcccgggactcgcctcctctctccctcccactcagggtgtggatttcaatgtgtgcagcctcctgggacctcagcagggc

SEQ ID NO: 88 - DNA, 120 bp, Artificial Sequence
  degradation probe
aaataaaaataaaaaataaaaaatccagtctgccccctgctcacaccggacaccagaataaactcccgatggggcgtacggcataaacccgggggaccccagctcccgggactcgcctcc

SEQ ID NO: 89 - DNA, 120 bp, Artificial Sequence
  degradation probe
cagggccccaccacgagtgccctcagtgcctcacctgaggccccagcgaacatcctcctgccagttttgtctgggggttttcttccccctttaaggccaggattctcaatcaagaggtga

SEQ ID NO: 90 - DNA, 120 bp, Artificial Sequence
  degradation probe
gaggtccctgtggccccagcaaagccggccgtggcaggtgtgtcccgtgggtgggtgagctggaaacggacagacagcaggacgtagcaggggacaggatgcagagctcgggaaggcagg

SEQ ID NO: 91 - DNA, 120 bp, Artificial Sequence
  degradation probe
cctcgtcagggaggcgtctccttgtgggtggcacgtgggcatgtgttgctctgagggctgtggccatgttgcccacctggccagggacccccgacttgggtgggtgacagccagcctccc

SEQ ID NO: 92 - DNA, 120 bp, Artificial Sequence
  degradation probe
caatgcccaggctgggtccccacttcctccttttgccttgggagcctgggagtcagaacggactccggggaggaggtgggggctttgggctcaaggtcccaacctttgtgggggcgggga

SEQ ID NO: 93 - DNA, 120 bp, Artificial Sequence
  degradation probe
attgggcaactcaacggcctctggcattgggctataagaggagcttgaccgtgggtgcaccctggaccccaccatggctcaccggccccccagccctgccctggcgtccgtgctgctggc

SEQ ID NO: 94 - DNA, 120 bp, Artificial Sequence
  degradation probe
cccagctgtgccagaagggctttggatggacctcgtggtggatatttccccacaatccaccggggcctggaggctggatggatgggcacgtggctcactcaccgctcagcagcaaggcca

SEQ ID NO: 95 - DNA, 120 bp, Artificial Sequence
  degradation probe
acagataggcaactgagtcaaatggccagggctcccccttgcccttgtcctccccacccctgcaagcgttggtcccgagcagtgagtggagtctcccctgaccctgcctcagtttcccca

SEQ ID NO: 96 - DNA, 120 bp, Artificial Sequence
  degradation probe
gcaaatgctcagtgaatatgcatgaatgaatgaatgagtgaatgaatgagtgagtgagtgaatgagtgaacaaatgaatgagtgaatgaatcaatgagtgagtgaatgaatgagtgaatg

SEQ ID NO: 97 - DNA, 120 bp, Artificial Sequence
  degradation probe
agtgaatgagtgaacgaatgaatgagtgaatgaatcaatgagtgagtgaatgaatgagtgaatgagtgaatgaatgagtgaatgagtgaatgagtgaacgaatgaatgagtgaatgaatg

SEQ ID NO: 98 - DNA, 119 bp, Artificial Sequence
  degradation probe
aaaaaagaaaaagaaaaagaaaatcaccctcaggatagggacctgagggggcccatgctctcttcttcccctgattccagcatcactaactcctcgaccctttcactcactcactcatt

SEQ ID NO: 99 - DNA, 120 bp, Artificial Sequence
  degradation probe
gtgagccactgcacccagccacctggtctgcgtcttaaaagccttcctgactctcaggactgaaagctgccaccagggcgcctttggaaatcgtcgtaattataacccccccggcctggg

SEQ ID NO: 100 - DNA, 120 bp, Artificial Sequence
  degradation probe
gggagcccccaggctgcagggaaaggggggatggcagagcagcccctgggcctctccgagcctccgtctcccgatctgcaacccggtcagtgcctgctggctggtgggaaggactcagcg

SEQ ID NO: 101 - DNA, 120 bp, Artificial Sequence
  degradation probe
gcccgggttcccccgcatctgcagggaggccatgtagggccgggagtgtggctgcgcctcgtgcccgcccacgatctccgcagctcgggcagcacctgcagggggggagtccaggcgtca

SEQ ID NO: 102 - DNA, 120 bp, Artificial Sequence
  degradation probe
agccacttctgcggaggcaccttgatccaccccagcttcgtgctgacggccgcgcactgcctgcgggacatgtgagcggccgcctccacacccctgtccgcccgccccgccctcttcctc

SEQ ID NO: 103 - DNA, 120 bp, Artificial Sequence
  degradation probe
ctcggcgaccctgccccgggcctccccccagaactgcccacgcgcgcggggcctggggtctgcagacggggcttagctgggtcctccccgggcagagggacagtggccgggccagggctg

SEQ ID NO: 104 - DNA, 120 bp, Artificial Sequence
  degradation probe
ccacgttcaccaggcgctggggtctgcgggggtggggtggtcactcctcggcgccctggacactcgccgccgcccacagccggcccttcccgaggccgcggtgcagccccagacccctcc

SEQ ID NO: 105 - DNA, 120 bp, Artificial Sequence
  degradation probe
tgctcggagcccacaacgtgcggacgcaggagcccacccagcagcacttctcggtggctcaggtgtttctgaacaactacgacgcggagaacaaactgaacgacgttctcctcatccagg

SEQ ID NO: 106 - DNA, 120 bp, Artificial Sequence
  degradation probe
ccatttctgtctccatagagtggggacagccccccatttccccatgacacacatgagatactaaagaggtgccctacaaagtcccatcaccactagatgatttttttttttttttttttg

SEQ ID NO: 107 - DNA, 120 bp, Artificial Sequence
  degradation probe
gagttggggctccgcggtggcggtcagcagcataggcccaggtcccatgtgacccaaggctgtctctgaacctccatttctgtctccatagagtggggacagccccccatttccccatga

SEQ ID NO: 108 - DNA, 120 bp, Artificial Sequence
  degradation probe
tcccccccttatgacctgtaaacccagtttctggcggccaaggatggggagacaggaggcaactgcttgagcggagttggggctccgcggtggcggtcagcagcataggcccaggtccca

SEQ ID NO: 109 - DNA, 120 bp, Artificial Sequence
  degradation probe
ctgcccctccactcccgactatgagaacatgtttgtgggccagccagcagccgagcaccagtgggatgaacaagggtaagtaagccactgagacacctgccaaagtggtgaataataagg

SEQ ID NO: 110 - DNA, 120 bp, Artificial Sequence
  degradation probe
ccgggagctgaacaaaccctgggctgctcaggatgggcccaagcccggtttaggcttgcagccacggtacggcagccggagcgcccccaagccccaagtggccgtgccatcctgcccctc

SEQ ID NO: 111 - DNA, 120 bp, Artificial Sequence
  degradation probe
agctcccggcttctggccactcggcatcgccagagtctccaggccagcacagggccagcgatggcaagtccaagaagcaggcacccgctgaccaccactgccccgatagttgcagaggcc

SEQ ID NO: 112 - DNA, 120 bp, Artificial Sequence
  degradation probe
cgacatccgccgagacaactgctctggccagaagcctctgctctgctgggacacaaccagctcccagcacaacctctctgccttcctggaggtcagctgcgcccctggcctggcctctgc

SEQ ID NO: 113 - DNA, 120 bp, Artificial Sequence
  degradation probe
tgaggtcctgaacgtgctacgcaaccccttgtctcgtgtggatggggcgctggccgcccgctgtgaccttgacctgcaggccgactgcaactgtgccctggagtcctggcacgacatccg

SEQ ID NO: 114 - DNA, 120 bp, Artificial Sequence
  degradation probe
aggacctcaagcttctgcaggtgggcaaagaaggtcactggaagctctcgcaggccgttcccagacaggtcaaggagaatcacgttgctggcccgcagagactggttgtgaggcaggctc

SEQ ID NO: 115 - DNA, 120 bp, Artificial Sequence
  degradation probe
ggcaggctcaggctgaggccactgaaattcaggcacgtggcactgaactccgcgttccagtccacatccgcggaggacacggtgcacgacggttctaggctgtctgactcccgcagcagc

SEQ ID NO: 116 - DNA, 120 bp, Artificial Sequence
  degradation probe
cgcagcagcagcggcaacagcagcgtccatgccagggtgccccccattcaagcaacctacgtagagacaccggaatcctagccctgacctcagccctgtggtcgccctcgcctccaccgc

SEQ ID NO: 117 - DNA, 120 bp, Artificial Sequence
  degradation probe
ctccaccgccagtgtttattattatagctcagaccagctcaagccgcttctgaaatggtaaaacgcagccccagtctggtcgcctcctttggctggtgggctgcctcagtctccccttct

SEQ ID NO: 118 - DNA, 120 bp, Artificial Sequence
  degradation probe
gcatagggtcccgtcctggccaacgagggcgccccaaatgttcaggacatagaagaaggggttaactggcccggatctcctcctcgccttccaagcccgctaagcactggggttatctac

SEQ ID NO: 119 - DNA, 120 bp, Artificial Sequence
  degradation probe
gcccccactccaccctatcccaggacttcccagcgacccgccgttctgggagataccgggagcgtgatcagggggcggggccgtttccaaggcaaccgcttatttgcatagggtcccgtc

SEQ ID NO: 120 - DNA, 120 bp, Artificial Sequence
  degradation probe
gcagaggcaaaagcgtggcagtgggacccaaaaggtaggactgaggctctagaacttgcacctgtgcagggactgcaaaccagacctgggaggaccctttcagcagcccccactccaccc

SEQ ID NO: 121 - DNA, 120 bp, Artificial Sequence
  degradation probe
agcgaccccagcagcgctgcggacggtgctggccgtggccgctgcggcccccgtgtccaggtgggccaggacgcagcctctgggcgccgtcgcttttccagcatcgcagaggcaaaagcg

SEQ ID NO: 122 - DNA, 120 bp, Artificial Sequence
  degradation probe
cctgcaagggggcggatctctgtcttcattcttcccctctgcactccacccagacaccagttcctcttccccctgagtcgctgggtgcagagccagaggaacgccagcgaccccagcagc

SEQ ID NO: 123 - DNA, 120 bp, Artificial Sequence
  degradation probe
gctaatagaggacatgaagtcactgggttctagcaagagggaagcgggactctggtccacccctccctcttgccctccatccacctctggtgagaaccttgaggccctgcaagggggcgg

SEQ ID NO: 124 - DNA, 120 bp, Artificial Sequence
  degradation probe
atgtcctctattagcaccccaaccgactgccacctcatcaactcgcttcttgatctcagcttggctggttcccagctcatgcagtcatttattcattcaacaagcatcacccacgctggg

SEQ ID NO: 125 - DNA, 120 bp, Artificial Sequence
  degradation probe
ccctcctcactgtcccctccagcatagaggtgccactgtgcaagaactcagtgggagaggcttggaggagcttggagagccaatgataggtttggggaatcttagtgaactcctactcat

SEQ ID NO: 126 - DNA, 120 bp, Artificial Sequence
  degradation probe
ggctccacatggacctcttcccccaccaccaattccttctatcctctccactcctaaccatgctatcccagccagctacattttcccaagctgctgggagccctcctcactgtcccctcc

SEQ ID NO: 127 - DNA, 120 bp, Artificial Sequence
  degradation probe
ctgggacctcagcctcctccccaagttgcccaccccagacaccaggctcactccagagggacttcttgagccagtgcccctctgcccacgcacctgccttggctccacatggacctcttc

SEQ ID NO: 128 - DNA, 120 bp, Artificial Sequence
  degradation probe
tgacaccaggttcaggtgaggccttctcccagcccacccctcctcagtatcatttttgtcactactacccctccagtctgcctcaggcccccatctgcttctgggacctcagcctcctcc

SEQ ID NO: 129 - DNA, 120 bp, Artificial Sequence
  degradation probe
ctcacctgaacctggtgtcaggtggaccaggtacaacctatgccagcccgactcactgtgccaccttaactgcgtgtgacaccatgtccacagctgtcaaatgcacagaggcttctccgg

SEQ ID NO: 130 - DNA, 119 bp, Artificial Sequence
  degradation probe
atgcacagaggcttctccgggatctgggaggctgggagtccactcccccacgttttcccctcttttcggcctctgtccttcacatcgccaaactccaagaaatggtcaaaggctttgaa

SEQ ID NO: 131 - DNA, 120 bp, Artificial Sequence
  degradation probe
cgtgctttgttctactggccctgttaaaactcaagccagatcacgtccacctgctcacagccctcctgtggctcctcctcactctcagcagaaactgaccttccccctcttatctcacct

SEQ ID NO: 132 - DNA, 120 bp, Artificial Sequence
  degradation probe
tctgctgtctccaggtggcggcgagggaaagcgcatcctgggggaaccctgggcgcttcctcgcgttagactcccccaggttgccaactggccagtggctggtttcggtacccaggcgtg

SEQ ID NO: 133 - DNA, 120 bp, Artificial Sequence
  degradation probe
cagagtcacgcccagcgctgcgcaggctgatcgccgcgccgcgcccccgccctcggtcgcaggtggctcgttccgggaattcctaagcggaaaccggtcccaagccccgcgccttcgctc

SEQ ID NO: 134 - DNA, 120 bp, Artificial Sequence
  degradation probe
attccacttcggaagttttcgccacctcccccacccgttcccaacttgtggttcggcccctgggcatccctagcccccgggtcagccctccggaaattctggctcttaaaggggccgagc

SEQ ID NO: 135 - DNA, 120 bp, Artificial Sequence
  degradation probe
ttcgggcctcctgtgtctcccagttagatgacctgaggaaccctctaaagtccccggaagggaagctctgccccctcagggatgcccaaggggaatcagggccaggaaaagttggaattc

SEQ ID NO: 136 - DNA, 120 bp, Artificial Sequence
  degradation probe
cgaagcgccccccctccacccggtccggaggaaccccagtggaagtggagaagtcaggcgccaccaacaagcctctcccagccaggactttgcttagactcgctcctcccggcagggcgc

SEQ ID NO: 137 - DNA, 120 bp, Artificial Sequence
  degradation probe
tgcaaccgagacccacttggagcgattcacctgttgagatagggcgggtcccgccgcgcacctgttccctccctgcccaaacccctctccccggctggcgatggacccgcctaggtgcgc

SEQ ID NO: 138 - DNA, 120 bp, Artificial Sequence
  degradation probe
gcgctccgtcccccagggcccgaaccaagtgactggcccgctgggagggcgcctgcggaccacaggtcccttccggcccccatctgctccggccaaaccaagcgcaccgagatccatgca

SEQ ID NO: 139 - DNA, 120 bp, Artificial Sequence
  degradation probe
ctccggtgaatggttcggcccaggtacacaagggtgggagccgagggctgggcttaggggtgagcggcggggaggggttcctctgcctccctccccggaagtgggctgacccaggtgcgc

SEQ ID NO: 140 - DNA, 120 bp, Artificial Sequence
  degradation probe
ggagcgcgcagcgggtggagtgtggctcggaggaccgcggcgggtcaagcacctttctcccccatatctgaaagcatgccctttgtccacgtcgtttacgctcattaaaacttccagaat

SEQ ID NO: 141 - DNA, 120 bp, Artificial Sequence
  degradation probe
gcgcgtcccttccaaagtaccttagggggcgcggcgcgcaccaaggccaggaggggtgcacgctcctccccttcccacttccgttccttgtccctactccaagtccgtcctgttgcattc

SEQ ID NO: 142 - DNA, 120 bp, Artificial Sequence
  degradation probe
gcgcgggccagacgcgcccagacggccgcgatggcgctgttggccggcgggctctccagagggctgggctcccacccggccgccgcaggccgggacgcggtcgtcttcgtgtggcttctg

SEQ ID NO: 143 - DNA, 120 bp, Artificial Sequence
  degradation probe
acccccgcttcccgcttcccgccgcctccaactccatccatccccctctacagttccctccggcgttccgcagcgtcagaggccccgtgccccgtacctgtgcaccaggtgctaagcaga

SEQ ID NO: 144 - DNA, 120 bp, Artificial Sequence
  degradation probe
tatcttccagataagggcgcggggagcgctccccggcacaaatggggaattcctctcactcccgcgcgcaacagatcgcccaagacccaggggatcggaaggtcccagcctctgagaccc

SEQ ID NO: 145 - DNA, 120 bp, Artificial Sequence
  degradation probe
tcccatcccggcttttcttttcccagagatcgggcggtcttccactccctccccacaaagagttccattctccctgccgctccgggtctcaccgtccagggagtttcacttcctgctatc

SEQ ID NO: 146 - DNA, 120 bp, Artificial Sequence
  degradation probe
acccgaggcctaggcattgttccttcgtctctgtccctctaggaagcgtctgatccagccctcccgtgccctctccccgttactccaaaacttagaaatccccgggtgcgcccaagtccc

SEQ ID NO: 147 - DNA, 120 bp, Artificial Sequence
  degradation probe
cagaagtcggtttcaagtccgtaaatccatcccaccagactcttacgcacgtataatccggagcccgcgggaggtgggtggggtgcgggggatggggagtccccagtcccacacccaccc

SEQ ID NO: 148 - DNA, 120 bp, Artificial Sequence
  degradation probe
tctgctggcaggctttcacctggatgggatatttgggtggtgatgaggtctttcccgagacacttttggttcagtcatttgaaatgactttagagtagggtgaggtggtgggaggctgat

SEQ ID NO: 149 - DNA, 120 bp, Artificial Sequence
  degradation probe
accgactacgaaggattccaggttacccggggccttttgaatttggagggaaaaccatggagttgtttgcctgaactgctttgccatggagggactaaagcccccacaatatctccatca

SEQ ID NO: 150 - DNA, 120 bp, Artificial Sequence
  degradation probe
caccagtcccctcttccctttgccattcacccacaacaaggggcctctgtgtggcctgggcaggcaagcccagatggccatgttgatgcccccagcgcccaaggccccacttcaaaaccg

SEQ ID NO: 151 - DNA, 119 bp, Artificial Sequence
  degradation probe
ggtgtggttcagaggccacaggctgggaagagggatggcgggcgagtccaaggaaactggccgtgtcaccgtgcacctgccacttcagccccacgggtctataaaatgggcatgattat

SEQ ID NO: 152 - DNA, 120 bp, Artificial Sequence
  degradation probe
gtgccaggcactctgtaaaccacatacttgcgagtgtcaagctggtgacaggtggcgttcctgttgaagcacctccctgagctcacagcaacccttgctgtctctcctcttgccctcagc

SEQ ID NO: 153 - DNA, 120 bp, Artificial Sequence
  degradation probe
tctctcctcttgccctcagctcctgccagggccatccaggtgaccgtgtccaacccctaccacgtggtgatcctcttccagcctgtgaccctgccctgtacctaccagatgacctcgacc

SEQ ID NO: 154 - DNA, 120 bp, Artificial Sequence
  degradation probe
cctaccagatgacctcgacccccacgcaacccatcgtcatctggaagtacaagtctttctgccgggaccgcatcgccgatgccttctccccggccagcgtcgacaaccagctcaatgccc

SEQ ID NO: 155 - DNA, 120 bp, Artificial Sequence
  degradation probe
cgacaaccagctcaatgcccagctggcagccgggaacccaggctacaacccctacgttgagtgccaggacagcgtgcgcaccgtcagggtcgtggccaccaagcagggcaacgctgtgac

SEQ ID NO: 156 - DNA, 120 bp, Artificial Sequence
  degradation probe
gccagtcccaccacccgggcaagcccagcctcatccccctgccctgcccaacatacttccggtgatggtaatcctccggccctggtagtaatctcccagggtcacagcgttgccctgctt

SEQ ID NO: 157 - DNA, 119 bp, Artificial Sequence
  degradation probe
gcccgggtggtgggactggcgtccttgtgcgggacctggagtccccatctgaaagctcttgagtgccagtgtctgaaaggaccattgaagggagcaattctttttttttttttttttga

SEQ ID NO: 158 - DNA, 120 bp, Artificial Sequence
  degradation probe
gtgtgcagttcctggcatgtcacaggctttctccattaagctctcttgcccctgcagttcactcctgccatctcctctgtgagtcctctgcggaccaccctaggcaagcaaagggattag

SEQ ID NO: 159 - DNA, 120 bp, Artificial Sequence
  degradation probe
gtaacctctctgtgcctcagctgtcaggagggtagaggagaggactcaatgggagaatatgtgtgcagttcctggcatgtcacaggctttctccattaagctctcttgcccctgcagttc

SEQ ID NO: 160 - DNA, 120 bp, Artificial Sequence
  degradation probe
cctcccaaagtgctgggatttgtttccggtttttttcctactatgctatacttttgactttgtttccctgactaggatgtcaattccgtgaggctggatttttgctgattttttttttta

SEQ ID NO: 161 - DNA, 120 bp, Artificial Sequence
  degradation probe
actatgctatacttttgactttgtttccctgactaggatgtcaattccgtgaggctggatttttgctgatttttttttttaacctctgtacccctagctttttttttttttttttttttt

SEQ ID NO: 162 - DNA, 120 bp, Artificial Sequence
  degradation probe
gcttcctccgcggcgctttaacacgcagggcgctgctgccaggggcgtcccgtgttctaactcgctcccacagcccctccggcttggtgagcagcgtctgaggggtgaggggcatagact

SEQ ID NO: 163 - DNA, 120 bp, Artificial Sequence
  degradation probe
gggggctctgtggatggcattcggggagctacaggtttcccccaaaagctcagatgctcgttcttgaagagggaggtgctgcccctgccttcctgcgtaccgcgacaatacagcttcctc

SEQ ID NO: 164 - DNA, 120 bp, Artificial Sequence
  degradation probe
gagcccccctcagtcggcctcccctgaggcaccaaggctggcggaagcagtcacctgtccatctcccccacttctcacagaggaaatcctgggtggtgccgggcaatgagtcagggctga

SEQ ID NO: 165 - DNA, 120 bp, Artificial Sequence
  degradation probe
cctaaggccaagggcgcctgggccctgcagaggagcggagcagggggaggagcgctgagacctgcccgttggaggaatgctgagacgccccacccaacctctgtcctggtcctcagccct

SEQ ID NO: 166 - DNA, 120 bp, Artificial Sequence
  degradation probe
ttggtgcaggcagcttctgggcttgagtccggccccctgcacctccagtccacactccccaggagctcacctgctcccaggtcgaactccatggcggtaagagaagttgggtcctaaggc

SEQ ID NO: 167 - DNA, 120 bp, Artificial Sequence
  degradation probe
tccgaccggccctgaactttgtggggactgagcttgggatctcccccgtggcccgcccccacaccgggcttctgggaggtgggctccagggctgtggagagaagttgggtggttggtgca

SEQ ID NO: 168 - DNA, 120 bp, Artificial Sequence
  degradation probe
tgaagttggaggcctaaggcaggaccctggggtcaggggcaaccccagccttcccgcccctccgcagccggtgatgaggcgacttacctttggacccggacctgcccctgcctccgaccg

SEQ ID NO: 169 - DNA, 120 bp, Artificial Sequence
  degradation probe
taccttcacatccgtgtccgaatcgctggagctgctgctggagtcggaagagctgtggtgtccttgctggatggaggtgcggcagtgaggcggcgccccttacccagccccctgaagttg

SEQ ID NO: 170 - DNA, 120 bp, Artificial Sequence
  degradation probe
ggctggaagcgagacagggggaccactccccgcaccctccccgccagccccagtgcggggacgcctctctggggtgcagggcacgtgcttggggacgctggcgagagccccttaccttca

SEQ ID NO: 171 - DNA, 120 bp, Artificial Sequence
  degradation probe
ttccagcccagaaccatctcttctctcccatccctgccctcggccccacagtcccacgctgctggctccaagcagcacgagagcatcccgggcaaggccaagaagcccaaagtgaagaag

SEQ ID NO: 172 - DNA, 120 bp, Artificial Sequence
  degradation probe
agaagggcaagaagaaggaggctccccactgaagggccctggacagggctcattaaaccttcctctctgccttctgcgactggtcagcgtggtgcctactctggcccgtccccagctccc

SEQ ID NO: 173 - DNA, 120 bp, Artificial Sequence
  degradation probe
ccagctccctgccccctgcccagcgctgggcccgcccattcgtgcgcgaggccaaggagaggctgtccacgccatgcccatcagggtttattgtttctgtaacagcggccacgccctggg

SEQ ID NO: 174 - DNA, 120 bp, Artificial Sequence
  degradation probe
ggccccaaggtccaccctgtctggccacaggcaccgcctccatcccatgtcccgcccagccccgcccccaacccaaggtgctgagagatctccagctgcacaggccaccgccccagggcg

SEQ ID NO: 175 - DNA, 120 bp, Artificial Sequence
  degradation probe
catgtactggctcaccttccacctggtgcggtgagcgcgcccgctgaacctcccgctgctgctgctgctgctgggggccactgtggccgccgaactcatctcctgcctgcaggccccaag

SEQ ID NO: 176 - DNA, 120 bp, Artificial Sequence
  degradation probe
cagtacatgagcataggctgcgccgccgccaccgccatggtcaggtacatgcgcagctggttccgggccccacgcaccgggaccccctcagctgctgcgtctgccaagatcttcagccgc

SEQ ID NO: 177 - DNA, 120 bp, Artificial Sequence
  degradation probe
ttcagccgcagcgtccggatctgggtgggaaagagagagaggaaccaagggatttacccttgagccctctgggccagagtgagagcccggacatacttccagcctctgcctagaagcccc

SEQ ID NO: 178 - DNA, 120 bp, Artificial Sequence
  degradation probe
agggcccggggagaagcaaatcctcaccatgttaggaggtttggggaaactgaggccccgggagcagaaacctgaggctgcagagggccagggacttgtccccagctgcctggggcttct

SEQ ID NO: 179 - DNA, 120 bp, Artificial Sequence
  degradation probe
gggccacgtctcccaccccctcaggccgggtgtccaccgggaagtcccccttcacccgctccaagaccttgcctggcatttgagtatggaaccttctcagattttgcccacagggcccgg

SEQ ID NO: 180 - DNA, 120 bp, Artificial Sequence
  degradation probe
acgtggcccaggatgctgcccccccttatgctggccacacggcaatttctgaagacacgccagaaatgctatctttatttttattaaaggacagggtctctggcctggtgcgttggctca

SEQ ID NO: 181 - DNA, 114 bp, Artificial Sequence
  degradation probe
tgtgagtcacgtgcttggcctaaacttttaaatgatgagacttaagtcaaagtttatccaaggctctgccctggcccaccagccaccacagtctgccctctgcttctcacatgc

SEQ ID NO: 182 - DNA, 120 bp, Artificial Sequence
  degradation probe
cagaatttccctggaaggagattctctagagcccttcccactggagtcaggggtgctggtgaagagcactggggtctgcaggctgggtggaccccaagcttagttggatcctgggcaaat

SEQ ID NO: 183 - DNA, 120 bp, Artificial Sequence
  degradation probe
ccaggctgctagaggctggggatccccagcacacaggctccaggctgggctctcactctcattccactctccttacatgggagccttcctgccagaatttccctggaaggagattctcta

SEQ ID NO: 184 - DNA, 120 bp, Artificial Sequence
  degradation probe
ctcctctccccacaggcctgtgtggcccagccatgcgctctcccacttgggtccctgccctggccgcggcctgggctacccacccaggtagccaaaggaatctttttttgttttgttttg

SEQ ID NO: 185 - DNA, 120 bp, Artificial Sequence
  degradation probe
gtgttagttattactctcaacatctcttcaaaccacctttatgcttgcctctctcagactcctcttcctcctctccccacaggcctgtgtggcccagccatgcgctctcccacttgggtc

SEQ ID NO: 186 - DNA, 120 bp, Artificial Sequence
  degradation probe
gagaggcaagcataaaggtggtttgaagagatgttgagagtaataactaacacttgttatctcattcaatcttcacaagatgcaatgaagaagtgggctttttttttttttgagacagag

SEQ ID NO: 187 - DNA, 120 bp, Artificial Sequence
  degradation probe
aaattcacctattattccagcttctctcttccaaatacggcctctgtgccctgtttcttcccccaggaggagatgaacggctggctggaggctgtagcttcctcggtggcggaacacgca

SEQ ID NO: 188 - DNA, 120 bp, Artificial Sequence
  degradation probe
ggggtgggaagtcacttcctgcgcccgctggccctgcgatctccgccttccctcttagggttgccctcatctgtggatgaagtagtgggtagtgtctggccccagcgggcgatctctgcg

SEQ ID NO: 189 - DNA, 120 bp, Artificial Sequence
  degradation probe
cccaccatgtcacgcgtcctcagtgttggaactaggggctcccgcccctgcggaagaaaaagtgtctttgtgcccacagtgcggaaaagaggggagacgagatgtgtcaggtcctggggg

SEQ ID NO: 190 - DNA, 120 bp, Artificial Sequence
  degradation probe
gacccccagcacctggggtgggggtggtgagtgggaaggggccgagatatggctataggggcagcaatctccccaccacttcttggggtgcaggagaagtcccctcctttccggtgccca

SEQ ID NO: 191 - DNA, 120 bp, Artificial Sequence
  degradation probe
tactgcccccaccaccccagcttcttggggcaggatctgcagatcggggctgggggcttggctcaactggccccctgcccacccccatcaagctcagttattgcataaaaataagggacc

SEQ ID NO: 192 - DNA, 120 bp, Artificial Sequence
  degradation probe
gtgaggtaaaggcggggagggggtgtcccctccctgccccctagcaccctggatggtccaggggacctcaccagcgcccccgtgcccatccctagggcagagaggggggcaggaattact

SEQ ID NO: 193 - DNA, 120 bp, Artificial Sequence
  degradation probe
ttttgcaaaaataaactaagtcaggaattaaccgcatggctctgaggcggcggagtgcagggggctagggccccttcactcccccctcccagacagagatcaaggcagcattggaagtga

SEQ ID NO: 194 - DNA, 120 bp, Artificial Sequence
  degradation probe
catgaagtggcatttacaactggaggggggaggagagaggaaaacagttttgagctttgacgcccctcccattaaaagccttcgcggatgcggggcgggggaggaggagatcgacgtttt

SEQ ID NO: 195 - DNA, 120 bp, Artificial Sequence
  degradation probe
ccccagcggcttctccaggcggccatggggactgcccacaacccaatcccccgactccccagggaggtgctccagccagaagcatgcaggggtgggcttcccctcgcccctcccctcatg

SEQ ID NO: 196 - DNA, 120 bp, Artificial Sequence
  degradation probe
aatcctcccgggcgcagcccagaaaacccaaacaaaggaccatagaccgtcaaagccaaagggtttattataggtacatacagtgtgcgcccgccacaccgccccacacccccgggcccc

SEQ ID NO: 197 - DNA, 120 bp, Artificial Sequence
  degradation probe
ccccgaaattaagtccacataaaagggtacatctggatgttctccgcagccacggtacccccgtgcccctctccccaccccgcacccacccctacagccaccacagccctcctttcaatc

SEQ ID NO: 198 - DNA, 120 bp, Artificial Sequence
  degradation probe
cgcctttcctgaacttgacggggcggacccggagtctcctttggggaagtttattacgaagcataacgaaatattctccatccctctaggttctcgtcaaggggcgtgtgtttagacccc

SEQ ID NO: 199 - DNA, 120 bp, Artificial Sequence
  degradation probe
gcacccggaccccgcctccataggctgctcattgggccagagactccaggagtcactagcgattggacagagcgcctgcccatcgcggttaacttttccgcctccttaaaggggtccgcc

SEQ ID NO: 200 - DNA, 120 bp, Artificial Sequence
  degradation probe
cattcagatgaatgacttccccgggaggcccccgactggggaccccctcggctgcagtagccgctgctgccatggcccccgggcgagccggctggcccgcacccacttccgggtgtgcac

SEQ ID NO: 201 - DNA, 120 bp, Artificial Sequence
  degradation probe
gcggagcaacccttgatggacagggattcccccaggagaggcggatactcccaccccgcttcccgcccccgaggatgggacctcaggagtctcccacactcacctcttgcctcccacatt

SEQ ID NO: 202 - DNA, 120 bp, Artificial Sequence
  degradation probe
agagatgaagaggagcgggtgaggaatgcgtccccctcccgcaggatccagggttccctccccttccccctcccccggcgtttttccagaatctcgcgcctccaaagggggggggggcgg

SEQ ID NO: 203 - DNA, 120 bp, Artificial Sequence
  degradation probe
ctcctaccctcgccccaaactcgctctcccgatcacctggagaagaaggagtctgggatccaggtgagagtctggcgagaggtactgaatctgggcggggaaggtaaacggacgcaagag

SEQ ID NO: 204 - DNA, 120 bp, Artificial Sequence
  degradation probe
ggagggatggagagggcgtgggagacctaatatccactcccccggcccgtgacgcctgctcggtgtcccttccctgcagtcttctgagcggacgcatctcgacgctgaaagatgagaccg

SEQ ID NO: 205 - DNA, 120 bp, Artificial Sequence
  degradation probe
gggaaaggggggcagaggatggatggggtaaaaggcaggactgagtggggtcctgcccctcttcctaccaacccctcccccccatcccatttcagttcttgtccgactcactgctccggt

SEQ ID NO: 206 - DNA, 120 bp, Artificial Sequence
  degradation probe
agacagtgagggactccctcccctttttccatgccaacctgggatccaactctttggtgcgcaggaagttgaggatgggggcgaagactgtagggtccctgtcgatgaagatctgcggga

SEQ ID NO: 207 - DNA, 112 bp, Artificial Sequence
  degradation probe
gtcttcagtccctgaggagttctttcacctcctctgaatgtccacgtgagctccattctattagaattctcattcagaaccttgatcacaggccgggcgcgccgggattgct

SEQ ID NO: 208 - DNA, 120 bp, Artificial Sequence
  degradation probe
acacacacacacacacacactcaacatttcctccacccatatcatcactccttagcatctttattccatcaaaactttctaccccttgacattctctgtgcagttttgaaaattaccctc

SEQ ID NO: 209 - DNA, 120 bp, Artificial Sequence
  degradation probe
gagtgagcccatagaactgggcttcatggaggaggctggaaccgtggacacccctgggtagtagaattgactagaggtcagggtctaggtgtgggggtgaacagagaatgctgagagggt

SEQ ID NO: 210 - DNA, 120 bp, Artificial Sequence
  degradation probe
ccacctctaccccagccctctttcaggagagaaggggaagaggggcaggtagcatcacagagttggttttctatacaatgaggatgatagctgggggctcccacttacccagaggagtga

SEQ ID NO: 211 - DNA, 120 bp, Artificial Sequence
  degradation probe
acttagggagatgctgaggccaccccatttccccctccccatttctttctacacagagtaccaatactcctcccagtacgaatacagacccggccagggccataaccagtcctcccacct

SEQ ID NO: 212 - DNA, 120 bp, Artificial Sequence
  degradation probe
ctaagtcccagcctttaagtcctcctgttgcagttcgtcgcctgcagcttcgagaggagttggatcgatcttcttgtggaaacgtcctcttcaatggttacctgccgccaccaggtaggc

SEQ ID NO: 213 - DNA, 120 bp, Artificial Sequence
  degradation probe
gaactacacctcccactgtatctcaggacgcacagcacaggcacccgagagctgcagccttatgggaaatgtagtctttggcccacctccgcgcccctccattccattgggagtgcctac

SEQ ID NO: 214 - DNA, 120 bp, Artificial Sequence
  degradation probe
tagttccctatcagatgcttgggctgatgcttggaaaggaagttggacacagcatttcccatgagacaatgggccagctaactcttgaggctcagaaggatgtcctggagccccatggga

SEQ ID NO: 215 - DNA, 120 bp, Artificial Sequence
  degradation probe
ggctatcatgggcattactgtcaataacttcctattaatatttctcccctccatacgaagaatcccaggtcttccaaagccccccctacaaagaaaccaaacccggactacaactcccat

SEQ ID NO: 216 - DNA, 120 bp, Artificial Sequence
  degradation probe
atagccagcccactggccatggaaggtatgccccagtggttattggaactaggcttttctgattggtagaagtaacagagtagggaaatttcatctacagctttatttccctaactgcag

SEQ ID NO: 217 - DNA, 120 bp, Artificial Sequence
  degradation probe
ctgcagtcagcacctgctaccttcatgaaagttgccagatataaagatctgtagtagtacttttccaacttagttttatcctgttttccgaaaaacaatcatttatttatttatttattt

SEQ ID NO: 218 - DNA, 120 bp, Artificial Sequence
  degradation probe
cattatatacaacccaaggcttgccacaaactgaaatttcttatgcattgacattcagcatttgatgtaaaatgacaatcttagagtcgctgccacaaaccctggtaaaactgcccctgc

SEQ ID NO: 219 - DNA, 120 bp, Artificial Sequence
  degradation probe
cttctccagttcactaagttcagctactccttcagggcttgtgctcctttaaaacttttcagaagccccttgaagtaccaacatgttgaaactcaaactcaactgcctgcagcttcagat

SEQ ID NO: 220 - DNA, 120 bp, Artificial Sequence
  degradation probe
cagctctggccactgagaattcttctccaggccccagtgtcaaaggcacttttgaccatcatgtgtttgtctgactaccaagttggactgccaaccttcactaaaaactccggcaccttt

SEQ ID NO: 221 - DNA, 120 bp, Artificial Sequence
  degradation probe
atctacctcctgtcagatcaccgcctgtcagagagggaggcctaaaaaggcttcaggatatcaacagctttgagctgccttagaggaacttcttttctattttgtagtacaggagcctca

SEQ ID NO: 222 - DNA, 120 bp, Artificial Sequence
  degradation probe
tgcactgcatcatccaaacaagactctccacaaaacattactaatggattctaattgaacagagtttggttacaagagacagtaaacacaaatgggcacctgttttaaatggcaaactta

SEQ ID NO: 223 - DNA, 120 bp, Artificial Sequence
  degradation probe
cacaataaattcaattgatcttaaaaactaaacattcctctaaggttactgactataaatatctagaaattactactcctatagtcttgctatttccagacaagtgaaactggaagggag

SEQ ID NO: 224 - DNA, 120 bp, Artificial Sequence
  degradation probe
agaggagaaatacccagttataatgacaagacaaccttatcttttctcaaaagcctccccaaacacacaaccattttccacaaaacagttacatcaaaaaagatgtagcaagacctagac

SEQ ID NO: 225 - DNA, 120 bp, Artificial Sequence
  degradation probe
attaagcatgctagaaagtagggacagttttaccagtgtttgtggcagcaactctgagatagtcattttacatcaaatgctaaatgtcaatgcataagaaatttcagtttgtggtaagcc

SEQ ID NO: 226 - DNA, 120 bp, Artificial Sequence
  degradation probe
ctgggttatatataatggcaacaagagaaaactatcacataatctgaattacgttcatttttctgcatacaccatgcatgctggtacactttcagttagcctaaacttcacccaaaagta

SEQ ID NO: 227 - DNA, 120 bp, Artificial Sequence
  degradation probe
gaggtggctatgaaaactctacctgcatggtataaatagatacctaaccctacatgcatgggtcacttgtatctacctcctaaataccaaaaacttagaagcccagatttcttttcagtc

SEQ ID NO: 228 - DNA, 119 bp, Artificial Sequence
  degradation probe
aaaaacaaaacaaaacaaaaaattacccttccccacaattataagcattttggcatatttccttcctgtctctgttgttcctattttggcagtatttttattgttatcgcaggcagcct

SEQ ID NO: 229 - DNA, 120 bp, Artificial Sequence
  degradation probe
gccgcacaccttatacctctcccctttaaaacagacatattaaagacttctgttttctgaatttactgtaaacagaatacttaaaaaaaaattacccagacaggcgtggtggctcactgt

SEQ ID NO: 230 - DNA, 120 bp, Artificial Sequence
  degradation probe
tgaacaaactgcattagactaaaatctttcacgagtccaaattctgcgactagaaaaataaacttgttagccgcacaccttatacctctcccctttaaaacagacatattaaagacttct

SEQ ID NO: 231 - DNA, 120 bp, Artificial Sequence
  degradation probe
acttactttcttgttctgactcaactcggaaaacacctccctagtctgaggcccccctccttatatttgtgaacaaactgcattagactaaaatctttcacgagtccaaattctgcgact

SEQ ID NO: 232 - DNA, 120 bp, Artificial Sequence
  degradation probe
tttccactatatcacactgcactgtctggctccaatctgatggcgagcaatggttccttaaaaagctccatcttctcaagcctacccagcacagacctacaactcaaactttaagtttta

SEQ ID NO: 233 - DNA, 120 bp, Artificial Sequence
  degradation probe
agccagcctaaccttccacgtccacttgaaaaaagaaaaaaaagaaagaaacaatcaaccggaagcctttgatttttttgctagcaagaagccaataattgttttctttgggacaatcac

SEQ ID NO: 234 - DNA, 120 bp, Artificial Sequence
  degradation probe
caggttaagttgtgaaatgggtggcagaaactgcaagatttaaccagtttctctcaacgctaatcaaaggtcaagaaaataaaagacagggcagtgagagtcgcgggactttagacactc

SEQ ID NO: 235 - DNA, 120 bp, Artificial Sequence
  degradation probe
agtgtggaaaccccttccccactcagtccaatttcaaggttatggagcataattaagagtgaccattctaaaagcgttctctagtgccctggctaagacttttaagcttcttggtgcagg

SEQ ID NO: 236 - DNA, 120 bp, Artificial Sequence
  degradation probe
agaagcaaagatgatgtctgtcacctggcagcgtagacggagagtggcggtagatgacttgaatggacacgtggattcgagtctggggacaggcgttcccccagtcggtgcgttgtgaga

SEQ ID NO: 237 - DNA, 120 bp, Artificial Sequence
  degradation probe
tcaatttcaaaaaatgccagcactccttagatcaatgactccacggccaagtgcaagggccacgggcactcctctggagtcaccgagagaatgtctgcctctggcagcggcctctgagct

SEQ ID NO: 238 - DNA, 120 bp, Artificial Sequence
  degradation probe
ggcccccgcctgcccagaggccgggctcgcgggcgttcggaccgcgcgtggggcgagactgcactcgcgcccgctgagatgccggccccacgcagcgcggcggcacgaggcccacaggca

SEQ ID NO: 239 - DNA, 120 bp, Artificial Sequence
  degradation probe
cgcggacgagggcaacgtcagcggcccagctgggccacctcctcccagccgaagcgcggcctccggctgcttcgccgggccggtccccgccgccgcacacacaagggtcggtcccgcgcg

SEQ ID NO: 240 - DNA, 120 bp, Artificial Sequence
  degradation probe
ctgcccgcggccccgcttccctcggcagccggaggcctccggggtgcccgtgctgccgcagcttcctgtgtctgagtcgcgggcggcgcgggcgcggggctggtgcgcggccggggatgc

SEQ ID NO: 241 - DNA, 119 bp, Artificial Sequence
  degradation probe
gggccccggccgaggcgccggctgacgttgcccttcagggccgagcccggagcagcggccgccgcggaggcgcgtgagccaccccaggctcgtggggccgcgccgcctttccggcctcc

SEQ ID NO: 242 - DNA, 120 bp, Artificial Sequence
  degradation probe
gccgcgggagcaggcgcggctccgcacggcgggcgcactcacctcctccgtggaccggccgctccgccgccggctgtcactgcagcccactccctgccccgcgccggctctggatgctat

SEQ ID NO: 243 - DNA, 120 bp, Artificial Sequence
  degradation probe
tctggatgctatttaaggcgccgctggcgccgccgcctcggcccgctggttgcgcccccgccaccgccgcggctgagccaggatgctgctggtgcgctcgccgcgccggccccgccctcc

SEQ ID NO: 244 - DNA, 120 bp, Artificial Sequence
  degradation probe
cgccgagcgcccggccgccctcctgtctgggcgcgcgtggctggcgggtggccggcggtcgagaggctgcggcgggcgaacgccgctggagtcgcccagactgcctcggatttcataatg

SEQ ID NO: 245 - DNA, 120 bp, Artificial Sequence
  degradation probe
caggatccggagggcgccgcccgcccctcgggcatcgcggggcccagggtccgcggggcctctgagctgaggcggtccaatgtaccctttgtccttcctcagattcattatgaaatccga

SEQ ID NO: 246 - DNA, 120 bp, Artificial Sequence
  degradation probe
gccctccggatcctgcaccctcagcccgtgccccgctggcctcctggcaggccgggcagaattctgctgcgagggccctggaggcaagggccttcctctccgtttttccttgacactgac

SEQ ID NO: 247 - DNA, 120 bp, Artificial Sequence
  degradation probe
ttccttgacactgacgggcgcggccccacagggtttcccccacgctccctggccggaagaccttttctgtctggtgtctgcagagcctgacgtgggcagcgcccgcagctcgcgccggtg

SEQ ID NO: 248 - DNA, 120 bp, Artificial Sequence
  degradation probe
tgggcatggcagggacgcggaagccctcagaaccgcggaagccctcagaaccgcacagcaggtggccgccgtggtgcagcgcgcagtcggccctcagctcccctgcaccggcgcgagctg

SEQ ID NO: 249 - DNA, 120 bp, Artificial Sequence
  degradation probe
gttgttagaggttgaggttcctggtaggtcccaggtgctaaaaggaagagcagcctttgggatgtgagcccgaaaaccatcgtgttggcttctgaagccccaagatgggcatggcaggga

SEQ ID NO: 250 - DNA, 120 bp, Artificial Sequence
  degradation probe
tcaacctctaacaacattggtccccgttaccgtggagtttgcgcccagcgttcgctggagggccgtttgaagaatgattgttgtcgtaaaggcctttgataataatagatactgtttacc

SEQ ID NO: 251 - DNA, 120 bp, Artificial Sequence
  degradation probe
gtcagtttcccctgcctgcagcccaggcatagccctgcgttggttgtgcccacactctgggggcagcctgagtgccaagtgtggtactgctctcactagcatttaacctctgtgccccag

SEQ ID NO: 252 - DNA, 120 bp, Artificial Sequence
  degradation probe
tgcgcccagcacactgtgggagacacccagggaaagtccttcagttatttgtgggagtgaactgcagtgtggctgaggcctggagcgaaccacggtcagtttcccctgcctgcagcccag

SEQ ID NO: 253 - DNA, 120 bp, Artificial Sequence
  degradation probe
ccattttttctagatctggaagatggagatctaaaaggactgagctgctagagaggttgctggtgtgtgcgttgtcctcaccccactgcaccactgcgcccagcacactgtgggagacac

SEQ ID NO: 254 - DNA, 120 bp, Artificial Sequence
  degradation probe
ccatcttccagatctagaaaaaatggactccacgttcgttttcactggttcccgcttggctaatccctggtttttctgtccagatctcgcctccccatccgtgattttgtcatctttgcc

SEQ ID NO: 255 - DNA, 120 bp, Artificial Sequence
  degradation probe
ccatccgtgattttgtcatctttgcctctcctttttcctcttctccaaccggtccctcagccccagaacctctgcaggcacctcccaagttctgtggtctattgttttcaagggctgctg

SEQ ID NO: 256 - DNA, 120 bp, Artificial Sequence
  degradation probe
tgacaattcaacccgtaacatgtggatggtcgtcactggctgtctcaccttacgtcttctcctcaagacacttttcttttaaactgggtgcctctccctgggctctagcccttaatgagg

SEQ ID NO: 257 - DNA, 120 bp, Artificial Sequence
  degradation probe
aagacacttttcttttaaactgggtgcctctccctgggctctagcccttaatgaggtgatttccaactcaaggctgggaaaggcacacaacccagtatcagacctgagttcacactcctt

SEQ ID NO: 258 - DNA, 120 bp, Artificial Sequence
  degradation probe
cccatacattgtaaaactttttggagtgattagataaaaacttttttgtttttttttaagtgtgtgtgctgaaagagacctaagagattatttagccaagcccccttatagcctgaggag

SEQ ID NO: 259 - DNA, 120 bp, Artificial Sequence
  degradation probe
tcttaaccactatgcaatactgcctcccatcaaagtctctatctcaagtccacaagacaaccccatacattgtaaaactttttggagtgattagataaaaacttttttgtttttttttaa

SEQ ID NO: 260 - DNA, 120 bp, Artificial Sequence
  degradation probe
gacaggtgtcataggattgacagccacgcccttggcgttctcattaaatgaagattccagactctatagttgggagtttgtgacttaggtaagaaaccagtctctttttttgagacggag

SEQ ID NO: 261 - DNA, 120 bp, Artificial Sequence
  degradation probe
tgtcataggattgacagccacgcccttggcgttctcattaaatgaagattccagactctatagttgggagtttgtgacttaggtaagaaaccagtctctttttttgagacggagtctcac

SEQ ID NO: 262 - DNA, 120 bp, Artificial Sequence
  degradation probe
ctctgtcgcccaggctggagtgcagtggcacaatctctgctcactgcaagctccgcctcccgggttcacgccattctcctgcctcagcctcccgagtagctgggactacaggcgcccgct

SEQ ID NO: 263 - DNA, 120 bp, Artificial Sequence
  degradation probe
gcccgcctcggcctcccaaagtgctgggattacaggcgtgagccaccgcgcccggcccacagctctagttctttaatacacatcaggtcggtcatggtggctcatgcctgtgatcccagc

SEQ ID NO: 264 - DNA, 120 bp, Artificial Sequence
  degradation probe
ttgctatgttgccctggctggtcttgaactgccagccttcagggacgctcctgccttgacctccccaaatgctgggatcacaggcatgagccaccatgaccgacctgatgtgtattaaag

SEQ ID NO: 265 - DNA, 120 bp, Artificial Sequence
  degradation probe
gagcgtccctgaaggctggcagttcaagaccagccagggcaacatagcaagaccccatttctacaaaaaattaaacagccaaccaaaaaaaaaaaaaaaaagaaagaaaaactttagctg

SEQ ID NO: 266 - DNA, 120 bp, Artificial Sequence
  degradation probe
cctgggctcaagcaattctcctgcctcaaccccccaagtagctgggaccacaggcatgctccaccatgtccagctaaagtttttctttctttttttttttttttttggttggctgtttaa

SEQ ID NO: 267 - DNA, 120 bp, Artificial Sequence
  degradation probe
gacagggtctggctgtgtcacccaggctggagtgcagtggtgtaatcatggctcactgcagcctcgacctcctgggctcaagcaattctcctgcctcaaccccccaagtagctgggacca

SEQ ID NO: 268 - DNA, 120 bp, Artificial Sequence
  degradation probe
catgattacaccactgcactccagcctgggtgacacagccagaccctgtctcaaaaaatatatatgtatattttaaaatattatatattatataatgtaaataatatacatgctacatat

SEQ ID NO: 269 - DNA, 120 bp, Artificial Sequence
  degradation probe
ttttaaaatattatatattatataatgtaaataatatacatgctacatattttatgtatacatctatgttatatacattaatatagccaggtgcagtggctcacacctgtgttcccagca

SEQ ID NO: 270 - DNA, 120 bp, Artificial Sequence
  degradation probe
atatacattaatatagccaggtgcagtggctcacacctgtgttcccagcactttggcgggattacgggcgggtcacctgaggtcaggagttcgagaccagcctggccaatatagtgaaac

SEQ ID NO: 271 - DNA, 120 bp, Artificial Sequence
  degradation probe
ggtcacctgaggtcaggagttcgagaccagcctggccaatatagtgaaaccccgtctttactaaaaatacaaaaaaaaaaaaaaattagccaggcgtggtggtgggcgtctataatccca

SEQ ID NO: 272 - DNA, 120 bp, Artificial Sequence
  degradation probe
aaaaaaaaaaaaaaattagccaggcgtggtggtgggcgtctataatcccagctatacaggaggctgaggcaggagaattgtttgaacccgggaggcggagattgcagtgagctgagatcg

SEQ ID NO: 273 - DNA, 120 bp, Artificial Sequence
  degradation probe
ttcccctttggatttttttttttttttgagacagagtctcgctctgtcaccaggctggagagcagtggtgcgatctcagctcactgcaatctccgcctcccgggttcaaacaattctcct

SEQ ID NO: 274 - DNA, 120 bp, Artificial Sequence
  degradation probe
gtgctgatggtaataaccttgaccacgtggtcagggcatctgccggttcctgcactgtccagttactgttttcccctttggatttttttttttttttgagacagagtctcgctctgtcac

SEQ ID NO: 275 - DNA, 120 bp, Artificial Sequence
  degradation probe
gggagtcccgggcgtggcgtgtgtctgtctcaggccccccacgtcgaagcccatggcttcggcctgtctcgtgctgatggtaataaccttgaccacgtggtcagggcatctgccggttcc

SEQ ID NO: 276 - DNA, 120 bp, Artificial Sequence
  degradation probe
actcaatggagggagtcccgggcgtggcgtgtgtctgtctcaggccccccacgtcgaagcccatggcttcggcctgtctcgtgctgatggtaataaccttgaccacgtggtcagggcatc

SEQ ID NO: 277 - DNA, 120 bp, Artificial Sequence
  degradation probe
cctctaaccctggggtgattctgcccccaggggtccctgtgtgaggcctggagacatccgtggttgtcacgacttggagggagctcctggcagggccctgcagtgcccaggatggccccg

SEQ ID NO: 278 - DNA, 120 bp, Artificial Sequence
  degradation probe
ctcagagtctcttggggtctgtggaatcctctaaccctggggtgattctgcccccaggggtccctgtgtgaggcctggagacatccgtggttgtcacgacttggagggagctcctggcag

SEQ ID NO: 279 - DNA, 120 bp, Artificial Sequence
  degradation probe
agggagccaagatcgtgccactgcactccagcctgggcaacagagcaagactccatctcaaaacataaataaataaaaataaaaaataaaaaatccagtctgccccctgctcacaccgga

SEQ ID NO: 280 - DNA, 120 bp, Artificial Sequence
  degradation probe
tcttgctctgttgcccaggctggagtgcagtggcacgatcttggctccctgcaaccttcgcctcccaggctcaagtgattctcctgcctcagcctcctgagtagctgggactacaggcgc

SEQ ID NO: 281 - DNA, 120 bp, Artificial Sequence
  degradation probe
accagcctggccaacatagtgaaaccccatctctactaaaaatacaaaaattagctgcgcgtgttggcgggcgcctgtagtcccagctactcaggaggctgaggcaggagaatcacttga

SEQ ID NO: 282 - DNA, 120 bp, Artificial Sequence
  degradation probe
gcctcacacctgtaatcccagcactttgggaggccgaggcgggtggatcgcctgaggtcaggagttcaagaccagcctggccaacatagtgaaaccccatctctactaaaaatacaaaaa

SEQ ID NO: 283 - DNA, 120 bp, Artificial Sequence
  degradation probe
cgatccacccgcctcggcctcccaaagtgctgggattacaggtgtgaggcaccgcacctggccatttttttccccaaataatgaacactggtctctcccctgggccctgtggacatcggg

SEQ ID NO: 284 - DNA, 120 bp, Artificial Sequence
  degradation probe
gtggaggccaaggatgatgcctagcaccctgcagtgcccaggacgggcctgccccagagaaggatccagccccgatgtccacagggcccaggggagagaccagtgttcattatttgggga

SEQ ID NO: 285 - DNA, 120 bp, Artificial Sequence
  degradation probe
aggcccgtcctgggcactgcagggtgctaggcatcatccttggcctccacccactccatgccaggagctcccctttcgtgacagccacaggtgtcctcagacctcgcccagggtcccctg

SEQ ID NO: 286 - DNA, 120 bp, Artificial Sequence
  degradation probe
tgggggttttcttccccctttaaggccaggattctcaatcaagaggtgattctgcccccaggggaccctgggcgaggtctgaggacacctgtggctgtcacgaaaggggagctcctggca

SEQ ID NO: 287 - DNA, 120 bp, Artificial Sequence
  degradation probe
gaattcgcctgttactagctaattcccaaaacagctcctcaggagggaagggctgggttttgcccgttttacagataggcaactgagtcaaatggccagggctcccccttgcccttgtcc

SEQ ID NO: 288 - DNA, 120 bp, Artificial Sequence
  degradation probe
cttccctcctgaggagctgttttgggaattagctagtaacaggcgaattcctggacagcacccagccttcagcaaatgctcagtgaatatgcatgaatgaatgaatgagtgaatgaatga

SEQ ID NO: 289 - DNA, 120 bp, Artificial Sequence
  degradation probe
cccctcaggtccctatcctgagggtgattttctttttctttttcttttttttttttttgagacggagtctcgccctgtcgcccaggctggagtgcagtggcgcgatctcggctcactgca

SEQ ID NO: 290 - DNA, 120 bp, Artificial Sequence
  degradation probe
cgccctgtcgcccaggctggagtgcagtggcgcgatctcggctcactgcaagctcagcctcccgggttcacgcctttctcctgcctcagcctcccgagtagctgggactacaggcgccca

SEQ ID NO: 291 - DNA, 120 bp, Artificial Sequence
  degradation probe
agaccatcctggctaacaccttgaaaccccgtctctacaaaaattacaaaaaattatccaggcatggtggtgggcgcctgtagtcccagctactcgggaggctgaggcaggagaaaggcg

SEQ ID NO: 292 - DNA, 120 bp, Artificial Sequence
  degradation probe
cggtggctcacgcctgtaatcccagcactttgggaggctgaggcaggcggatcacgaagtcaggagatcgagaccatcctggctaacaccttgaaaccccgtctctacaaaaattacaaa

SEQ ID NO: 293 - DNA, 120 bp, Artificial Sequence
  degradation probe
ccgcctgcctcagcctcccaaagtgctgggattacaggcgtgagccaccgcgcctggccctttttttttcttttttgaaacggaatcttgctctgtcgcccaggctggagtgcagtggtg

SEQ ID NO: 294 - DNA, 120 bp, Artificial Sequence
  degradation probe
gctactcaagaggcggaggcaggagaatcatttgaccccaggaggctgaggctgcagtaaactgagattgcaccactgcactccagcctgggcgacagagcaagattccgtttcaaaaaa

SEQ ID NO: 295 - DNA, 120 bp, Artificial Sequence
  degradation probe
gtggtgaaaccccatctctactaaaaatacaaaaactagccaggtgtggtggtgtgtgcttgtaatcccagctactcaagaggcggaggcaggagaatcatttgaccccaggaggctgag

SEQ ID NO: 296 - DNA, 120 bp, Artificial Sequence
  degradation probe
tttgcagatgaacaaactgaggcattgaaaatccccctcaggtcaggagttcgagaccagcctgaccaacgtggtgaaaccccatctctactaaaaatacaaaaactagccaggtgtggt

SEQ ID NO: 297 - DNA, 120 bp, Artificial Sequence
  degradation probe
aacaaaacaaaacaaaataaaaaaaaagaagcaaaaaaaaaaaaaaaaatcagccaacaaagagacccattttgcagatgaacaaactgaggcattgaaaatccccctcaggtcaggagt

SEQ ID NO: 298 - DNA, 120 bp, Artificial Sequence
  degradation probe
aaaaaaaaaaaaaaaaaaaaaaaatcctgggcgcagtggctcatgcctgagtgagaccccatctcaaaaaaacaaaacaaaacaaaataaaaaaaaagaagcaaaaaaaaaaaaaaaaat

SEQ ID NO: 299 - DNA, 120 bp, Artificial Sequence
  degradation probe
tcaggcatgagccactgcgcccaggattttttttttttttttttttttttgagacagaatctcactctgttgcccaggctggagtgcagtggtgtgatctcggctcactgcaacctgtgc

SEQ ID NO: 300 - DNA, 120 bp, Artificial Sequence
  degradation probe
tgcccaggctggagtgcagtggtgtgatctcggctcactgcaacctgtgcctcctgggttcaagcgattctcctgcctcagcctcccaggcagctgagattacaggtgcgaaccaccaca

SEQ ID NO: 301 - DNA, 120 bp, Artificial Sequence
  degradation probe
tcctgcctcagcctcccaggcagctgagattacaggtgcgaaccaccacacctggctaattttttttttttttttttttgagacggagtgtcattctgttgccaggctggagtgcagtgg

SEQ ID NO: 302 - DNA, 120 bp, Artificial Sequence
  degradation probe
tactccggaggttgacgcaggagaatcgcttgaacccaggaagcggaggttgtggtgagccaagatcgccccactgcactccagcctggcaacagaatgacactccgtctcaaaaaaaaa

SEQ ID NO: 303 - DNA, 120 bp, Artificial Sequence
  degradation probe
acctccgcttcctgggttcaagcgattctcctgcgtcaacctccggagtagctgggactacaggcacgtgccaccacgcccagctaatgtttgtatttttagtagagacagggtttcacc

SEQ ID NO: 304 - DNA, 120 bp, Artificial Sequence
  degradation probe
ccaccacgcccagctaatgtttgtatttttagtagagacagggtttcaccatgttggccaggatggtctccatctcttgacctcaggtgatccacccacctcggcctcccaaagtattgg

SEQ ID NO: 305 - DNA, 120 bp, Artificial Sequence
  degradation probe
catctcttgacctcaggtgatccacccacctcggcctcccaaagtattgggattacaggcgtgggccaaccgcacccgaccaatttttctgcttctttaaaaacatttttttaaattttg

SEQ ID NO: 306 - DNA, 120 bp, Artificial Sequence
  degradation probe
cgcacccgaccaatttttctgcttctttaaaaacatttttttaaattttgttttagagacagggtcttgctctgttgcccaggctggagtgcagtggtgtgatctcagttcactgcagcc

SEQ ID NO: 307 - DNA, 120 bp, Artificial Sequence
  degradation probe
tctgttgcccaggctggagtgcagtggtgtgatctcagttcactgcagccttgacctcctgggctcaagcgatcctccctcctcagcctcccaagtagctgggacgacaggtgcacacca

SEQ ID NO: 308 - DNA, 120 bp, Artificial Sequence
  degradation probe
tcaagaccagcctgggcaaaattgtgaggaccccatctctacaaaaaattttaaaattagccaggcatggtggtgtgcacctgtcgtcccagctacttgggaggctgaggagggaggatc

SEQ ID NO: 309 - DNA, 120 bp, Artificial Sequence
  degradation probe
gcagtggctcacgcctgtaatcccaacactttgggaggccgaggcaggaggctcccttgagctcaggagttcaagaccagcctgggcaaaattgtgaggaccccatctctacaaaaaatt

SEQ ID NO: 310 - DNA, 120 bp, Artificial Sequence
  degradation probe
aggctggtcttgaactcctgagctcaagggagcctcctgcctcggcctcccaaagtgttgggattacaggcgtgagccactgcacccagccacctggtctgcgtcttaaaagccttcctg

SEQ ID NO: 311 - DNA, 120 bp, Artificial Sequence
  degradation probe
gaggtggaatcacttgagcctgcagtgagctgagattgtgtcactgcattacagcctgggcaacagagggagaccctctcaaaaaaataaaaggcacgcacgcccccaaacctgaaatga

SEQ ID NO: 312 - DNA, 120 bp, Artificial Sequence
  degradation probe
aatgcagtgacacaatctcagctcactgcaggctcaagtgattccacctcagcctcccgagttgcctggattataggcacacaccatcatgcctggctaatttttgggtttttgtttttt

SEQ ID NO: 313 - DNA, 120 bp, Artificial Sequence
  degradation probe
tgagcaacatattgagacctcatctctaccaaaaacaacaaaaacaacaaaaaaaacccctaaaaaacacaaaaaacaaaaacccaaaaattagccaggcatgatggtgtgtgcctataa

SEQ ID NO: 314 - DNA, 120 bp, Artificial Sequence
  degradation propbe
ttgttgtttttgttgtttttggtagagatgaggtctcaatatgttgctcaggctgttctccaacttctgggctcaagccatcctcctgcctcccaaagtgctgggattacagatatgaac

SEQ ID NO: 315 - DNA, 120 bp, Artificial Sequence
  degradation probe
caaccccaagaatagaaaaggcactcactggccagtcgaagtggttcatatctgtaatcccagcactttgggaggcaggaggatggcttgagcccagaagttggagaacagcctgagcaa

SEQ ID NO: 316 - DNA, 120 bp, Artificial Sequence
  degradation probe
gctctgaaggaaaagcaggccaagtgggtttcttcctttttttttttttttgagatagaatctcttgctctgtcacccgggctggagtgcaatggcacaatcttggctcactgcaatctc

SEQ ID NO: 317 - DNA, 120 bp, Artificial Sequence
  degradation probe
tgtcacccgggctggagtgcaatggcacaatcttggctcactgcaatctccgcctcccgagttcaagcaattctcctgcctcagcctcctgagtagctgggattacaggcgcgtgacacc

SEQ ID NO: 318 - DNA, 120 bp, Artificial Sequence
  degradation probe
ttctcctgcctcagcctcctgagtagctgggattacaggcgcgtgacaccatgcctagctaatttttgtatatttttagtagagacagggtttcaccatgttggtaaggctggtcttgaa

SEQ ID NO: 319 - DNA, 120 bp, Artificial Sequence
  degradation probe
tatttttagtagagacagggtttcaccatgttggtaaggctggtcttgaactcctgacctcgtgatccgctggcctcggcctcctaaagtgctgggattacaggtgggagccaccgcacc

SEQ ID NO: 320 - DNA, 120 bp, Artificial Sequence
  degradation probe
tggcctcggcctcctaaagtgctgggattacaggtgggagccaccgcaccaggcctctttttttttttgagacggagtcttgctctgtcgcccaggctggagtgcagtggcgcgatctcg

SEQ ID NO: 321 - DNA, 120 bp, Artificial Sequence
  degradation probe
gacggagtcttgctctgtcgcccaggctggagtgcagtggcgcgatctcggctcactgcaagctctgcctcctgggttcacgccattctcctgcctcagtctcccgagtagctgggacta

SEQ ID NO: 322 - DNA, 120 bp, Artificial Sequence
  degradation probe
cctgggttcacgccattctcctgcctcagtctcccgagtagctgggactacaggcacctgccactacgcctggctaattttttgtatttttagtagagacacagtttcaccgtttctacc

SEQ ID NO: 323 - DNA, 120 bp, Artificial Sequence
  degradation probe
aaggaatgaggagccccaggaggtcttggtaagagataaaaaaacaggagggggccctctctggctctatggtagaaacggtgaaactgtgtctctactaaaaatacaaaaaattagcca

SEQ ID NO: 324 - DNA, 120 bp, Artificial Sequence
  degradation probe
acctgtaattccagcactttgggaggccgaggcgggcggatcacaaggtcaggagatcgagaccatcctgaaggaatgaggagccccaggaggtcttggtaagagataaaaaaacaggag

SEQ ID NO: 325 - DNA, 120 bp, Artificial Sequence
  degradation probe
gaccttgtgatccgcccgcctcggcctcccaaagtgctggaattacaggtatgagccaccgcacccggccttttttttttttttttgagaagcagtctctgttgcccagactggagtgca

SEQ ID NO: 326 - DNA, 120 bp, Artificial Sequence
  degradation probe
ttttttttttttttttgagaagcagtctctgttgcccagactggagtgcagtggcacgatctcagcccactggaacctctgcctcccggcttcaagcaattctcctaccttggccgccca

SEQ ID NO: 327 - DNA, 120 bp, Artificial Sequence
  degradation probe
tggaacctctgcctcccggcttcaagcaattctcctaccttggccgcccaagtagctgggattgcaggcgcccaccaccatgcccagctaattttttttttttttttttttagacggagt

SEQ ID NO: 328 - DNA, 120 bp, Artificial Sequence
  degradation probe
cccaccaccatgcccagctaattttttttttttttttttttagacggagtttcgctcttgttacccaggctggagtgcaagggcgcgatctcgactcaccacaacctccgcctcctgggt

SEQ ID NO: 329 - DNA, 120 bp, Artificial Sequence
  degradation probe
tggagtgcaagggcgcgatctcgactcaccacaacctccgcctcctgggttcaagcgattctcctgtctcagcctcccaagtagctgggtttacagccatgtgccaccacgcccagctaa

SEQ ID NO: 330 - DNA, 120 bp, Artificial Sequence
  degradation probe
ctgaggtcgggagtttgagaccagcctgaccaacatggagaaaccccgtttctactaaaaaaatacaaaattagctgggcgtggtggcacatggctgtaaacccagctacttgggaggct

SEQ ID NO: 331 - DNA, 120 bp, Artificial Sequence
  degradation probe
acttgaggccaggcgcggtggctcacgcctgtaatcccagcactttgggaggctgaggtgggcggatcacctgaggtcgggagtttgagaccagcctgaccaacatggagaaaccccgtt

SEQ ID NO: 332 - DNA, 120 bp, Artificial Sequence
  degradation probe
aagaaatagcccctcagaaacccacttgaggccaggcgcggtggctcacgcctgtaatcccagcactttgggaggctgaggtgggcggatcacctgaggtcgggagtttgagaccagcct

SEQ ID NO: 333 - DNA, 120 bp, Artificial Sequence
  degradation probe
ggaggtcagacttcagaaaggacttccctcttttatttttttttctgagacggagtcttgctctgtcgcccaggctggagtgcagtggtacaatctcagctcactgcaacctccacctcc

SEQ ID NO: 334 - DNA, 120 bp, Artificial Sequence
  degradation probe
caggctggagtgcagtggtacaatctcagctcactgcaacctccacctcctgggttcaagcgattcttctgcctcggcctcccaagtagctgggactacaagcacctgccaccacgcccg

SEQ ID NO: 335 - DNA, 120 bp, Artificial Sequence
  degradation probe
tgaggtcagttccagaccatcctggccaatatggtgaaaccccgtctctattaaaaatacaaaaataagccgggcgtggtggcaggtgcttgtagtcccagctacttgggaggccgaggc

SEQ ID NO: 336 - DNA, 120 bp, Artificial Sequence
  degradation probe
aagtcctggccgggtgtggtggctcatgcctgtaatcccagcactttgggaggccgaggtaggcggatcatgaggtcagttccagaccatcctggccaatatggtgaaaccccgtctcta

SEQ ID NO: 337 - DNA, 120 bp, Artificial Sequence
  degradation probe
tggccaggatggtctggaactgacctcatgatccgcctacctcggcctcccaaagtgctgggattacaggcatgagccaccacacccggccaggacttctctcttgagaagaaagggaaa

SEQ ID NO: 338 - DNA, 120 bp, Artificial Sequence
  degradation probe
taaaatatgactttcggggattgttatggtattaatgaggtagtggcaggtacatagtaagtgctcaataaatggtagttcctcatattattgccccagactattgctaaccctttcctt

SEQ ID NO: 339 - DNA, 120 bp, Artificial Sequence
  degradation probe
ttgtaagtggcagggctgagatctgaacccagtctggctcctgaccacaatgttacactgcccctcaaaataaaatatgactttcggggattgttatggtattaatgaggtagtggcagg

SEQ ID NO: 340 - DNA, 120 bp, Artificial Sequence
  degradation probe
ttgtggtcaggagccagactgggttcagatctcagccctgccacttacaagtattgcgggttggtcagccgggtgcagtggctcacgcctgtaatcctagcactttgggaggccgaggcg

SEQ ID NO: 341 - DNA, 120 bp, Artificial Sequence
  degradation probe
ttttagtagagacagggtttccccatgttggccaggctggtctggaactccagaccttaagtgatcccgccgcctcggcctcccaaagtgctaggattacaggcgtgagccactgcaccc

SEQ ID NO: 342 - DNA, 120 bp, Artificial Sequence
  degradation probe
tctcctgtctcagcctcccgagtagctgggattacaggcacgtgccaccacacccagataatttttgaatttttagtagagacagggtttccccatgttggccaggctggtctggaactc

SEQ ID NO: 343 - DNA, 120 bp, Artificial Sequence
  degradation probe
tggtggcacgtgcctgtaatcccagctactcgggaggctgagacaggagaatcacttgaacccaggaagcagaggttgcagtaagccgagactgtgccactgcactccagcctgggcaac

SEQ ID NO: 344 - DNA, 120 bp, Artificial Sequence
  degradation probe
tctagggaccccaacccaaccctcaatacttttttttttttttttttttttttagacagagtcttgctctgttgcccaggctggagtgcagtggcacagtctcggcttactgcaacctct

SEQ ID NO: 345 - DNA, 120 bp, Artificial Sequence
  degradation probe
caacagagcgagactccgtctcaaaaaacaaacaaacattgaacaccccaacttcttctcagcgtctgcttctagggaccccaacccaaccctcaatacttttttttttttttttttttt

SEQ ID NO: 346 - DNA, 120 bp, Artificial Sequence
  degradation probe
aggaggcggaggttgtaggaaggcggagattgcagtgagccaagatcgctccactgcactccagcctgggcaacagagcgagactccgtctcaaaaaacaaacaaacattgaacacccca

SEQ ID NO: 347 - DNA, 120 bp, Artificial Sequence
  degradation probe
agcgatcttggctcactgcaatctccgccttcctacaacctccgcctcctgggttcaagcgattctcctgcctcagcctcctgagtagctgggactacaggtgtgcaccaccacacccag

SEQ ID NO: 348 - DNA, 120 bp, Artificial Sequence
  degradation probe
cctcagcctcctgagtagctgggactacaggtgtgcaccaccacacccagctaatttttgtatttttagtagagacagggtttcatcatgttggctaggatggtctcgatctcttgacct

SEQ ID NO: 349 - DNA, 120 bp, Artificial Sequence
  degradation probe
agagacagggtttcatcatgttggctaggatggtctcgatctcttgaccttgtgatccgccctcctaggcctcccaaagtgctggtattacaggcgtgagccaccgcgcccagctcaatg

SEQ ID NO: 350 - DNA, 120 bp, Artificial Sequence
  degradation probe
ctcccaaagtgctggtattacaggcgtgagccaccgcgcccagctcaatgtttttttacaaatcagcacctgtgaaaggaagggagaggaaacataattgggcagagggagaagtcaaac

SEQ ID NO: 351 - DNA, 120 bp, Artificial Sequence
  degradation probe
gtgctcaagcaaggtgaggcaatcacagagctccccgcccttgacccaggtttgtccagcctgtgttgctgtttgacttctccctctgcccaattatgtttcctctcccttcctttcaca

SEQ ID NO: 352 - DNA, 120 bp, Artificial Sequence
  degradation probe
tcagcgtctgcctctggtgcagacagcggcctcactggaagtcatgccttttctgggcagcctacacccagtgctcaagcaaggtgaggcaatcacagagctccccgcccttgacccagg

SEQ ID NO: 353 - DNA, 120 bp, Artificial Sequence
  degradation probe
ccttcaaggaaggacttgccccaagctgcagggagtgcaggctgcagacaggctccagctgtcagctccttcagcgtctgcctctggtgcagacagcggcctcactggaagtcatgcctt

SEQ ID NO: 354 - DNA, 120 bp, Artificial Sequence
  degradation probe
ggaggtcaaatgacttgcccaagataacagggctagtgttgtaaacacagagatgtgctggccagatcagccttcaaggaaggacttgccccaagctgcagggagtgcaggctgcagaca

SEQ ID NO: 355 - DNA, 120 bp, Artificial Sequence
  degradation probe
ctgtgtttacaacactagccctgttatcttgggcaagtcatttgacctccaaagcgccttgtctgtaaaaacaggtaaaatagtaatgggattcatggcattcttatgaggattaaatga

SEQ ID NO: 356 - DNA, 120 bp, Artificial Sequence
  degradation probe
acaggtaaaatagtaatgggattcatggcattcttatgaggattaaatgaggtaatatgtgtaaagcttttaacgacactgcctggcacatagtaagcattcagtacactgtaaacatga

SEQ ID NO: 357 - DNA, 120 bp, Artificial Sequence
  degradation probe
taacgacactgcctggcacatagtaagcattcagtacactgtaaacatgaaccattagtatctccactttacaactgaggcttagaggcaaagtagtttacaaaaggccacaaagtctga

SEQ ID NO: 358 - DNA, 120 bp, Artificial Sequence
  degradation probe
aagcattcagtacactgtaaacatgaaccattagtatctccactttacaactgaggcttagaggcaaagtagtttacaaaaggccacaaagtctgacttcaattcccttgccctaagcac

SEQ ID NO: 359 - DNA, 120 bp, Artificial Sequence
  degradation probe
ggacccttctaccacatactaaaattaaaatggcccaggccgggtgcagtggctcacacctatgtgagcactttgggaagccaaggcaggtagattacttgaggtcaggagttcgagacc

SEQ ID NO: 360 - DNA, 120 bp, Artificial Sequence
  degradation probe
ctttgggaagccaaggcaggtagattacttgaggtcaggagttcgagaccagcctggccaacatggtgaaaccccatctcttctaaaaatacaaaaattaacccagtgtggtggcatgca

SEQ ID NO: 361 - DNA, 120 bp, Artificial Sequence
  degradation probe
accccatctcttctaaaaatacaaaaattaacccagtgtggtggcatgcacctgtaatcccaaatactcgggaggctgaggcatgagaatcggttgaacccggcaggtggaggttgcagt

SEQ ID NO: 362 - DNA, 120 bp, Artificial Sequence
  degradation probe
ggaggctgaggcatgagaatcggttgaacccggcaggtggaggttgcagtaagcggagatcacaccactgcactccaacctgggcgacaaagtgagactcagtctcaaaaaataaaatga

SEQ ID NO: 363 - DNA, 120 bp, Artificial Sequence
  degradation probe
cactccaacctgggcgacaaagtgagactcagtctcaaaaaataaaatgaaataaaataaagtggctcaaagactcaatttaagagttaaaactataaaactcttagaagaaaatattgt

SEQ ID NO: 364 - DNA, 120 bp, Artificial Sequence
  degradation probe
agactcaatttaagagttaaaactataaaactcttagaagaaaatattgttgaaaatcttcatcacattggacttggcaacaactgcataaataggataccaaaagtacaaggaagaaaa

SEQ ID NO: 365 - DNA, 120 bp, Artificial Sequence
  degradation probe
gacttggcaacaactgcataaataggataccaaaagtacaaggaagaaaaaatagtaggtaaattgaacttcatcaaattaagaacttttgtgcaccaaaggacactatcaagaaagtga

SEQ ID NO: 366 - DNA, 120 bp, Artificial Sequence
  degradation probe
tcatcaaattaagaacttttgtgcaccaaaggacactatcaagaaagtgaaaaaacctggagaatggaagaaaatatttgcagatcatatatctaacaagggattaatattcaaaatata

SEQ ID NO: 367 - DNA, 120 bp, Artificial Sequence
  degradation probe
aaaatatttgcagatcatatatctaacaagggattaatattcaaaatatatatagaactcctacaacttgacaacaaaaaaacaaacaacccaattcaaaaatgggcaaagtacttgaat

SEQ ID NO: 368 - DNA, 120 bp, Artificial Sequence
  degradation probe
acaacaaaaaaacaaacaacccaattcaaaaatgggcaaagtacttgaatagacacttctccaaagaagatatactaatggacaataaattcgtggaaatatgctcaacatcgttagtca

SEQ ID NO: 369 - DNA, 120 bp, Artificial Sequence
  degradation probe
ttcttgattatggccatcctggttagtgtgaagtggcatctcattgtggttgtaatctgcatttccccaatgactaacgatgttgagcatatttccacgaatttattgtccattagtata

SEQ ID NO: 370 - DNA, 120 bp, Artificial Sequence
  degradation probe
gtacattcccaccagcaatgcacggaggattccaatttctccacatccccaccaaaacttattatccattttcttgattatggccatcctggttagtgtgaagtggcatctcattgtggt

SEQ ID NO: 371 - DNA, 120 bp, Artificial Sequence
  degradation probe
gtgacttggtagttctatttttaaccttttgaggaactgccaaactgttttccccaatgactgcactattgtacattcccaccagcaatgcacggaggattccaatttctccacatcccc

SEQ ID NO: 372 - DNA, 120 bp, Artificial Sequence
  degradation probe
aaacagtttggcagttcctcaaaaggttaaaaatagaactaccaagtcacccagcaattccattcttaggcatatattcaaaagaaatgaaagcagatatttgtacaccagtgttcacag

SEQ ID NO: 373 - DNA, 120 bp, Artificial Sequence
  degradation probe
catatattcaaaagaaatgaaagcagatatttgtacaccagtgttcacagctgcactatttacaatagtcaaaaggtagaaacaacctaggtccatccacaaatgaatggataaataaaa

SEQ ID NO: 374 - DNA, 120 bp, Artificial Sequence
  degradation probe
aaaaggtagaaacaacctaggtccatccacaaatgaatggataaataaaacgtagcatatacatacaatggtacactagtccgctgtaaaaagaaattttgatcttactgcatgctacat

SEQ ID NO: 375 - DNA, 120 bp, Artificial Sequence
  degradation probe
gtacactagtccgctgtaaaaagaaattttgatcttactgcatgctacatggcttcgacatactacaacatggatggaccttgaaaacattattctttgtgaaataaactagacacagga

SEQ ID NO: 376 - DNA, 120 bp, Artificial Sequence
  degradation probe
tggatggaccttgaaaacattattctttgtgaaataaactagacacaggacaaatgttagacgattccacttatatgaggcacctagaatgggcaatttggtaagcaaagtagaatagaa

SEQ ID NO: 377 - DNA, 120 bp, Artificial Sequence
  degradation probe
atccccaacataaactctgtgaccattaaacagtaactccccattccctgctacctgtgcccctagtaatttctattctactttgcttaccaaattgcccattctaggtgcctcatataa

SEQ ID NO: 378 - DNA, 120 bp, Artificial Sequence
  degradation probe
tcagtggcattaagtatattcacaatgtcgtgtaccaatcaccactctttatccccgaaactgtttcatcatccccaacataaactctgtgaccattaaacagtaactccccattccctg

SEQ ID NO: 379 - DNA, 120 bp, Artificial Sequence
  degradation probe
aaagagtggtgattggtacacgacattgtgaatatacttaatgccactgaattttacacttgaagtggttaaagcgataaatattatagtttgcatattttatcataaaaatattttttt

SEQ ID NO: 380 - DNA, 120 bp, Artificial Sequence
  degradation probe
aaagcgataaatattatagtttgcatattttatcataaaaatatttttttaaacgatgaagggacgtgaacgggttgaaattttataaaaagtggccagggaaggtgtcactgcaatggt

SEQ ID NO: 381 - DNA, 120 bp, Artificial Sequence
  degradation probe
cgggttgaaattttataaaaagtggccagggaaggtgtcactgcaatggtgtcctacaggaggaggaagatcatgtggacatctgcgggaagggtgttctggcagagggagtagcacggg

SEQ ID NO: 382 - DNA, 120 bp, Artificial Sequence
  degradation probe
tcatgtggacatctgcgggaagggtgttctggcagagggagtagcacgggcgatggctctgaggactgtgagaagtatagttggaaacagcgaggaggccagggtgtccgaagctgagta

SEQ ID NO: 383 - DNA, 120 bp, Artificial Sequence
  degradation probe
ctcctcctcactctcagcagaaactgaccttccccctcttatctcacctcctcccactctctctggcttactcagcttcggacaccctggcctcctcgctgtttccaactatacttctca

SEQ ID NO: 384 - DNA, 120 bp, Artificial Sequence
  degradation probe
cgtgcacctgccacttcagccccacgggtctataaaatgggcatgattatcgtggctacctcactggtcctggcaattaaggaacaatgtgtgccaggcactctgtaaaccacatacttg

SEQ ID NO: 385 - DNA, 120 bp, Artificial Sequence
  degradation probe
ccccacgggtctataaaatgggcatgattatcgtggctacctcactggtcctggcaattaaggaacaatgtgtgccaggcactctgtaaaccacatacttgcgagtgtcaagctggtgac

SEQ ID NO: 386 - DNA, 120 bp, Artificial Sequence
  degradation probe
tgtctgaaaggaccattgaagggagcaattctttttttttttttttttgaagatggagtcttgctctggactccaggctggagtgcagtggtgcgatctcagctcactgcaacctccacc

SEQ ID NO: 387 - DNA, 120 bp, Artificial Sequence
  degradation probe
ctccaggctggagtgcagtggtgcgatctcagctcactgcaacctccacctcccaggttcaagcaattctcttgcctcagcctcccgagtagctgggactccaggtgcgtgccaccacgc

SEQ ID NO: 388 - DNA, 120 bp, Artificial Sequence
  degradation probe
caggagtttgagaccagcctggccaacatggtgaaaccccatctctactaaaaatacaaaaattaactgggcgtggtggcacgcacctggagtcccagctactcgggaggctgaggcaag

SEQ ID NO: 389 - DNA, 120 bp, Artificial Sequence
  degradation probe
cgctccctttaggctgggtatggtgtctctcagcactttgcgaggccaaggcgggcagatcatttgaggtcaggagtttgagaccagcctggccaacatggtgaaaccccatctctacta

SEQ ID NO: 390 - DNA, 120 bp, Artificial Sequence
  degradation probe
cttggcctcgcaaagtgctgagagacaccatacccagcctaaagggagcgattctattctactattcttccttctgctaatccttccattctttaatttaataacgaagattttttgagt

SEQ ID NO: 391 - DNA, 120 bp, Artificial Sequence
  degradation probe
cttctgctaatccttccattctttaatttaataacgaagattttttgagtacctgtcatataccaggtgctgttctgggccctgggaatacagctgttaacaaaatcatcaaaccacttc

SEQ ID NO: 392 - DNA, 120 bp, Artificial Sequence
  degradation probe
tgttctgggccctgggaatacagctgttaacaaaatcatcaaaccacttccctcgtggagcccacattgcagtgagagagacaaacacgacacacactctcaagtccttgaagataaaga

SEQ ID NO: 393 - DNA, 120 bp, Artificial Sequence
  degradation probe
agtgagagagacaaacacgacacacactctcaagtccttgaagataaagaaaactgggtaacggagagaagaggccagggtttgttctataatcattaataacacgagcagtaagaagta

SEQ ID NO: 394 - DNA, 120 bp, Artificial Sequence
  degradation probe
gaggccagggtttgttctataatcattaataacacgagcagtaagaagtaaaatttatctaagtaacaacttataaagggtctactgtgtgctaagctctcatccaggttcccaaggatt

SEQ ID NO: 395 - DNA, 120 bp, Artificial Sequence
  degradation probe
ttataaagggtctactgtgtgctaagctctcatccaggttcccaaggattaactcagaccacacagtaattgaatagattctatcattgtcatcttacagaggcccagagagagaaagtg

SEQ ID NO: 396 - DNA, 120 bp, Artificial Sequence
  degradation probe
cttggccactagacccaaagtggctgagttagaatcccagccccgttaccagctatgacactaggcaagtcactttctctctctgggcctctgtaagatgacaatgatagaatctattca

SEQ ID NO: 397 - DNA, 120 bp, Artificial Sequence
  degradation probe
ctcctctgtgagtcctctgcggaccaccctaggcaagcaaagggattaggagcttggccactagacccaaagtggctgagttagaatcccagccccgttaccagctatgacactaggcaa

SEQ ID NO: 398 - DNA, 120 bp, Artificial Sequence
  degradation probe
ggttctcagggctgcctgggttcaaaccccagctccagactttgacttcctatgcacccatttgaacaaggtaacctctctgtgcctcagctgtcaggagggtagaggagaggactcaat

SEQ ID NO: 399 - DNA, 120 bp, Artificial Sequence
  degradation probe
agccgagatcccgccactgcactccagcctgggcgacagagcgagactccgtctcaaaaaaaaagaacaaggttctcagggctgcctgggttcaaaccccagctccagactttgacttcc

SEQ ID NO: 400 - DNA, 120 bp, Artificial Sequence
  degradation probe
gccacaccagccggaccacccgccagaggctcaacggcccctcctcgcagccgaggaaactgaggctcggaggcagggcgcagaggcccaaggtgacacagcaggcggccgtcggccggg

SEQ ID NO: 401 - DNA, 120 bp, Artificial Sequence
  degradation probe
ggcccgcgctccccggccgacggccgcctgctgtgtcaccttgggcctctgcgccctgcctccgagcctcagtttcctcggctgcgaggaggggccgttgagcctctggcgggtggtccg

SEQ ID NO: 402 - DNA, 120 bp, Artificial Sequence
  degradation probe
gcagcaaatcatgggctgtcctagccccttttcagatgagaaaggagcccagagaggggagagggcctgccaaggtggcacatctggccaggcgctgcgtaggctgagccctgggggtca

SEQ ID NO: 403 - DNA, 120 bp, Artificial Sequence
  degradation probe
agacgtccagttttttgggaggggtgggacacaggagtctgttttttaactagctccccagagcacctgagcagcaaatcatgggctgtcctagccccttttcagatgagaaaggagccc

SEQ ID NO: 404 - DNA, 120 bp, Artificial Sequence
  degradation probe
aagaaatgcagacgtccagttttttgggaggggtgggacacaggagtctgttttttaactagctccccagagcacctgagcagcaaatcatgggctgtcctagccccttttcagatgaga

SEQ ID NO: 405 - DNA, 120 bp, Artificial Sequence
  degradation probe
gaggctgaggtgggtgaatcacttgaggtcaggagttcgagaccagcctgaccaatatggtgaaaccccatctcaaaaaaaaaaaaaattggggctaacggtgagcctgttttgcaggat

SEQ ID NO: 406 - DNA, 120 bp, Artificial Sequence
  degradation probe
caggctggtctcgaactcctgacctcaagtgattcacccacctcagcctcccaaagtgctgggattataggcgtgagccactgcacccagcctagccccatttttcacatgaagagaccg

SEQ ID NO: 407 - DNA, 120 bp, Artificial Sequence
  degradation probe
aggctggtctcgaactcctgacctcaagtgattcacccacctcagcctcccaaagtgctgggattataggcgtgagccactgcacccagcctagccccatttttcacatgaagagaccga

SEQ ID NO: 408 - DNA, 120 bp, Artificial Sequence
  degradation probe
aggcagaagttgtgatgagctgagatcccgccactgcactccagactgggcaacagagcaggactctgtaaaaaacaaaacaaaacaaaaaattacccttccccacaattataagcattt

SEQ ID NO: 409 - DNA, 120 bp, Artificial Sequence
  degradation probe
cccagtctggagtgcagtggcgggatctcagctcatcacaacttctgcctcctgagttcaagcgattctcctggctcagcctctcgagtagctgggactagagatgcatgccaccacgcc

SEQ ID NO: 410 - DNA, 120 bp, Artificial Sequence
  degradation probe
ctggctcagcctctcgagtagctgggactagagatgcatgccaccacgcctggctaattatttgtatttttagtagagatagggtttctccatgttggtcaggctggtctcgaactcctg

SEQ ID NO: 411 - DNA, 120 bp, Artificial Sequence
  degradation probe
tagtagagatagggtttctccatgttggtcaggctggtctcgaactcctgacctcaaatgatctgcccgcctcggtctcccaaagtgctgggattacagtgagccaccacgcctgtctgg

SEQ ID NO: 412 - DNA, 120 bp, Artificial Sequence
  degradation probe
ttggtcaggctggtctcgaactcctgacctcaaatgatctgcccgcctcggtctcccaaagtgctgggattacagtgagccaccacgcctgtctgggtaatttttttttaagtattctgt

SEQ ID NO: 413 - DNA, 120 bp, Artificial Sequence
  degradation probe
ggctgagccaggatgctgctggtgcgctcgccgcgccggccccgccctcccgcggccccgccccgcccgcaggccccgcccccggcaggccccgccccgccgagcgcccggccgccctcc

SEQ ID NO: 414 - DNA, 120 bp, Artificial Sequence
  degradation probe
ctcgccgcgccggccccgccctcccgcggccccgccccgcccgcaggccccgcccccggcaggccccgccccgccgagcgcccggccgccctcctgtctgggcgcgcgtggctggcgggt

SEQ ID NO: 415 - DNA, 120 bp, Artificial Sequence
  degradation probe
agaatgattgttgtcgtaaaggcctttgataataatagatactgtttacccagtgcttgatgccaggcattgctacaaacactgcgtacacattattctatttaatcttcataacccagt

SEQ ID NO: 416 - DNA, 120 bp, Artificial Sequence
  degradation probe
tcactagcatttaacctctgtgccccagcttcctcttctgtgaaaggacagtaatgacagtacctacctcactgggttatgaagattaaatagaataatgtgtacgcagtgtttgtagca

SEQ ID NO: 417 - DNA, 120 bp, Artificial Sequence
  degradation probe
gtgccaagtgtggtactgctctcactagcatttaacctctgtgccccagcttcctcttctgtgaaaggacagtaatgacagtacctacctcactgggttatgaagattaaatagaataat

SEQ ID NO: 418 - DNA, 120 bp, Artificial Sequence
  degradation probe
tctgcaggcacctcccaagttctgtggtctattgttttcaagggctgctggaacgaagtaccacacactaagtggcttcaacaaccgaaatgtatggtctgtcctggttctggagactag

SEQ ID NO: 419 - DNA, 120 bp, Artificial Sequence
  degradation probe
agtggcttcaacaaccgaaatgtatggtctgtcctggttctggagactagaagtacaaaatcacggcgtgggagggctggctgcttccgaggcccatgaaagagcatgtgtgcccggcca

SEQ ID NO: 420 - DNA, 120 bp, Artificial Sequence
  degradation probe
ggagggctggctgcttccgaggcccatgaaagagcatgtgtgcccggccactctgacttgcagatgcccatcttctccctatgtctcctcacaccatcttccctgtggtgtgtgtgtgca

SEQ ID NO: 421 - DNA, 120 bp, Artificial Sequence
  degradation probe
tcttctccctatgtctcctcacaccatcttccctgtggtgtgtgtgtgcaaatcccttctttctataaggacaccagtcatactggactagggcccacccgaattgcctcattttaactt

SEQ ID NO: 422 - DNA, 120 bp, Artificial Sequence
  degradation probe
acaccagtcatactggactagggcccacccgaattgcctcattttaacttgatgacctctgtaatcatgaggtaatgtccaagtaaggtcccattctaagatacctgaatgcccaggcac

SEQ ID NO: 423 - DNA, 120 bp, Artificial Sequence
  degradation probe
ggtaatgtccaagtaaggtcccattctaagatacctgaatgcccaggcacggtggttcatgcctgtaatcccagcactttgggaggccgaggcaggcagatcaactgaggtcaggagttc

SEQ ID NO: 424 - DNA, 120 bp, Artificial Sequence
  degradation probe
ccaccatgccccgctaatttttttatttttagtagagacagggtttcaccatgttggccaggctggtcttgaactcctgacctcagttgatctgcctgcctcggcctcccaaagtgctgg

SEQ ID NO: 425 - DNA, 120 bp, Artificial Sequence
  degradation probe
cctccacctccctgggttcaaacaattcttctgcctcagcctcccgagtagctgggactacaggtacgcaccaccatgccccgctaatttttttatttttagtagagacagggtttcacc

SEQ ID NO: 426 - DNA, 120 bp, Artificial Sequence
  degradation probe
tactcgggaggctgaggcagaagaattgtttgaacccagggaggtggaggttgcaatgagccaagatcacgccactgcattccagcctgggagacagagcaagactctgtctcaaaacaa

SEQ ID NO: 427 - DNA, 120 bp, Artificial Sequence
  degradation probe
ttacgggttgaattgtcacccccacattccccaagttcatatgttcaggggtttgttgttgttgttgttgttgttttgagacagagtcttgctctgtctcccaggctggaatgcagtggc

SEQ ID NO: 428 - DNA, 120 bp, Artificial Sequence
  degradation probe
ggtgagacagccagtgacgaccatccacatgttacgggttgaattgtcacccccacattccccaagttcatatgttcaggggtttgttgttgttgttgttgttgttttgagacagagtct

SEQ ID NO: 429 - DNA, 120 bp, Artificial Sequence
  degradation probe
aggctgggaaaggcacacaacccagtatcagacctgagttcacactccttctggcaggaacttctaggtgacattgggaagcaacttcattccttagaacctagatttctcctcaggcta

SEQ ID NO: 430 - DNA, 120 bp, Artificial Sequence
  degradation probe
aaagagacctaagagattatttagccaagcccccttatagcctgaggagaaatctaggttctaaggaatgaagttgcttcccaatgtcacctagaagttcctgccagaaggagtgtgaac

SEQ ID NO: 431 - DNA, 120 bp, Artificial Sequence
  degradation probe
aggtattattcctgatttaaggaggatgcaatgaggcttgatgtggattcacccaccccaaagcctgtgttcttaaccactatgcaatactgcctcccatcaaagtctctatctcaagtc

SEQ ID NO: 432 - DNA, 120 bp, Artificial Sequence
  degradation probe
atttaatgagaacgccaagggcgtggctgtcaatcctatgacacctgtctgaggtaggtattattcctgatttaaggaggatgcaatgaggcttgatgtggattcacccaccccaaagcc

SEQ ID NO: 433 - DNA, 120 bp, Artificial Sequence
  degradation probe
gcatgaacccaggaggtagagattgcagtgagccgagatcgcaccactgcaccccagcctgggcaacagagtgagactccgtctcaaaaaaagagactggtttcttacctaagtcacaaa

SEQ ID NO: 434 - DNA, 120 bp, Artificial Sequence
  degradation probe
gcagtggtgcgatctcggctcactgcaatctctacctcctgggttcatgccattttcctgcctcagcctcccaagtagctgggaccacaggcacccaccaccacacccggctagtttttt

SEQ ID NO: 435 - DNA, 120 bp, Artificial Sequence
  degradation probe
ctcggctcactgcaatctctacctcctgggttcatgccattttcctgcctcagcctcccaagtagctgggaccacaggcacccaccaccacacccggctagttttttgtatttttagtag

SEQ ID NO: 436 - DNA, 120 bp, Artificial Sequence
  degradation probe
agcctggccaacatggtgaaaccccctctctactaaaaatacaaaaattagctgggcatggtggtgggtgcctataatcccagctactcgggaagctgaggcaggacaatcatttgaacc

SEQ ID NO: 437 - DNA, 120 bp, Artificial Sequence
  degradation probe
ggtgcagtggctcatgcctgtaatctcagcactttgggaggccgaggcaggcggtcaagagttcgagaccagcctggccaacatggtgaaaccccctctctactaaaaatacaaaaatta

SEQ ID NO: 438 - DNA, 120 bp, Artificial Sequence
  degradation probe
ctgcctcggcctcccaaagtgctgagattacaggcatgagccactgcaccaggcctgtcttcaggtcttttttattttattatttatttatttatttatttatttatttatttattttga

SEQ ID NO: 439 - DNA, 120 bp, Artificial Sequence
  degradation probe
tttattttattatttatttatttatttatttatttatttatttattttgagacagagtctcgctctgtcgcccaggctggagtgccgtggtgcgatctcggctcactgcaagctccacct

SEQ ID NO: 440 - DNA, 120 bp, Artificial Sequence
  degradation probe
cccaggctggagtgccgtggtgcgatctcggctcactgcaagctccacctcccgggttcacgccattctcctgtctcagcctcccgagtagctgggattacaggcgcctgccaccatgcc

SEQ ID NO: 441 - DNA, 120 bp, Artificial Sequence
  degradation probe
aggaaatcgagaccatcctggccaacttagtgaaaccccatctctactaaaaatacaaaaaaattagctgggcatggtggcaggcgcctgtaatcccagctactcgggaggctgagacag

SEQ ID NO: 442 - DNA, 120 bp, Artificial Sequence
  degradation probe
ggccgggtgcagtggctcatgcctgtaatcccagcactttgggaggccgaggcaggcagatcacgaggtcaggaaatcgagaccatcctggccaacttagtgaaaccccatctctactaa

SEQ ID NO: 443 - DNA, 120 bp, Artificial Sequence
  degradation probe
tcggcctcccaaagtgctgggattacaggcatgagccactgcacccggcccacatactacattttctatccactcaccagttgatgaagacttaatttgtttccagttttcttttttctt

SEQ ID NO: 444 - DNA, 120 bp, Artificial Sequence
  degradation probe
cactcaccagttgatgaagacttaatttgtttccagttttcttttttcttttttttgagatgggatctccctgtattgcccaggttggtcttgaactcctgggctcaagtgatcctccca

SEQ ID NO: 445 - DNA, 120 bp, Artificial Sequence
  degradation probe
tgagatgggatctccctgtattgcccaggttggtcttgaactcctgggctcaagtgatcctcccacctaagcctcccaaagtgctgggatttgtttccggtttttttcctactatgctat

SEQ ID NO: 446 - DNA, 120 bp, Artificial Sequence
  degradation probe
ttttttttttaacctctgtacccctagctttttttttttttttttttttttttgagatggattcttgctctgtcgcccaggctggagtgcagcagtgtgatcttggctcactgcaacctc

SEQ ID NO: 447 - DNA, 120 bp, Artificial Sequence
  degradation probe
tgtcgcccaggctggagtgcagcagtgtgatcttggctcactgcaacctccaccccccgggttcaagcaattctcctgcctcagcctcccaagtagctgggattacaggcgtacgccacc

SEQ ID NO: 448 - DNA, 120 bp, Artificial Sequence
  degradation probe
ttgagaccagcctagccaacatggtcaaaccccgtctctactaaaaaaaatacaaaaattagccaggcatggtggcgtacgcctgtaatcccagctacttgggaggctgaggcaggagaa

SEQ ID NO: 449 - DNA, 120 bp, Artificial Sequence
  degradation probe
cagtggctcacacctgtaattcccagcactttgggaggctgaggcaggcggatcacctgaggttaggagtttgagaccagcctagccaacatggtcaaaccccgtctctactaaaaaaaa

SEQ ID NO: 450 - DNA, 120 bp, Artificial Sequence
  degradation probe
cgcctgcctcagcctcccaaagtgctgggaattacaggtgtgagccactgtgctcagcctgtgtgttttgagacagggtcttgcttcgtcacccaggctggagtgcagtggctcaatcac

SEQ ID NO: 451 - DNA, 120 bp, Artificial Sequence
  degradation probe
agacagggtcttgcttcgtcacccaggctggagtgcagtggctcaatcacagcttactgcagcctggacttcccaggctcaagtgatcctcccgcctcagcctctcaagtagctggaact

SEQ ID NO: 452 - DNA, 120 bp, Artificial Sequence
  degradation probe
tcccaggctcaagtgatcctcccgcctcagcctctcaagtagctggaactacaggcgagtgccaccatgcctggctaagttttaattttatgtagagatggtctcagtatgttgcccagg

SEQ ID NO: 453 - DNA, 120 bp, Artificial Sequence
  degradation probe
ctggctaagttttaattttatgtagagatggtctcagtatgttgcccaggctgatttcaaactcttgggcccaagtgatcctcctgccttgcttccctcccaaagtgctaggattacagg

SEQ ID NO: 454 - DNA, 120 bp, Artificial Sequence
  degradation probe
ccaagtgatcctcctgccttgcttccctcccaaagtgctaggattacaggcatgaaccacttcacctggcccatgctatatgtttgcccctcgtcttcttacctactccctcagttttct

SEQ ID NO: 455 - DNA, 120 bp, Artificial Sequence
  degradation probe
cttcagcacaggagtttggggaagacacaaacattcaacccctactgttccaacccctgcttcacagatgagaaaactgagggagtaggtaagaagacgaggggcaaacatatagcatgg

SEQ ID NO: 456 - DNA, 120 bp, Artificial Sequence
  degradation probe
attaataagggctccacccacatcacctgatgacctcccagaggtcctacctccaaataccatcactcagcttcagcacaggagtttggggaagacacaaacattcaacccctactgttc

SEQ ID NO: 457 - DNA, 120 bp, Artificial Sequence
  degradation probe
gtaggacctctgggaggtcatcaggtgatgtgggtggagcccttattaatgggatttgtgcccttatagaaagggacacaacatgagatgatcttgctctgggccatgccgggaggaagg

SEQ ID NO: 458 - DNA, 120 bp, Artificial Sequence
  degradation probe
aagggacacaacatgagatgatcttgctctgggccatgccgggaggaagggagaaggtggctgtcaacgagccaggaagcggcactcctgacactggatctaccaacacccctatctcag

SEQ ID NO: 459 - DNA, 120 bp, Artificial Sequence
  degradation probe
gccaggaagcggcactcctgacactggatctaccaacacccctatctcagacttccagcctccaggaccgtgagaactgtttcctgttgaagccaccctctctatatttgttacagcagc

SEQ ID NO: 460 - DNA, 120 bp, Artificial Sequence
  degradation probe
tgagaactgtttcctgttgaagccaccctctctatatttgttacagcagcccacactgattaagacactttttttttttttttttttttttttttttttgagatggagcctcgctccatc

SEQ ID NO: 461 - DNA, 120 bp, Artificial Sequence
  degradation probe
tttttttttttttttttttttttttttttgagatggagcctcgctccatcacccaggctggagtgcaatggcgcaatctcggctcactgcaacctctgcctcccgggttcaagtgattat

SEQ ID NO: 462 - DNA, 120 bp, Artificial Sequence
  degradation probe
gcgcaatctcggctcactgcaacctctgcctcccgggttcaagtgattatcctgggtcagcctcccaagtagctgggattgcaggcacctgccaccatggctggctaaattttgtatttt

SEQ ID NO: 463 - DNA, 120 bp, Artificial Sequence
  degradation probe
agctgggattgcaggcacctgccaccatggctggctaaattttgtatttttagtagagacagggtttcaccatgttggccaggctggtctccaactcctgacctcaggtgatccacctgc

SEQ ID NO: 464 - DNA, 120 bp, Artificial Sequence
  degradation probe
catgttggccaggctggtctccaactcctgacctcaggtgatccacctgcctcggcctcccaaagtgctgggcgtgagccaccatgcccggcctgacacttatcttttatatctatatat

SEQ ID NO: 465 - DNA, 120 bp, Artificial Sequence
  degradation probe
ggcgtgagccaccatgcccggcctgacacttatcttttatatctatatatttttttctttgttgagacggagtctcactgtgtcgcccaggctggagtgcagtggtgtgatcttggctca

SEQ ID NO: 466 - DNA, 120 bp, Artificial Sequence
  degradation probe
agtctcactgtgtcgcccaggctggagtgcagtggtgtgatcttggctcaccgaaacctctgcctcccaggttcaagtgattctcgtgcctcagcctccggagcagctgggattacaggc

SEQ ID NO: 467 - DNA, 120 bp, Artificial Sequence
  degradation probe
gttcaagtgattctcgtgcctcagcctccggagcagctgggattacaggcgtgagccaccatgactggcctaaaacacctaccttttattttttaaaaaaccaacacattatcagtactt

SEQ ID NO: 468 - DNA, 120 bp, Artificial Sequence
  degradation probe
taaaacacctaccttttattttttaaaaaaccaacacattatcagtacttcatgccagacaccattctaggaaattaactcctctgactttcacatcaatcccatgggtagctgttattt

SEQ ID NO: 469 - DNA, 120 bp, Artificial Sequence
  degradation probe
gaaattaactcctctgactttcacatcaatcccatgggtagctgttatttacagatgaggaaactgaggcacagaggtaaaacaactttgctgaggtcaccttgctgctaagtgacacag

SEQ ID NO: 470 - DNA, 120 bp, Artificial Sequence
  degradation probe
acagaggtaaaacaactttgctgaggtcaccttgctgctaagtgacacagctggcatctagtgcagcaggctggctccaagtctatgcccctcacccctcagacgctgctcaccaagccg

SEQ ID NO: 471 - DNA, 120 bp, Artificial Sequence
  degradation probe
agcccctccggcttggtgagcagcgtctgaggggtgaggggcatagacttggagccagcctgctgcactagatgccagctgtgtcacttagcagcaaggtgacctcagcaaagttgtttt

SEQ ID NO: 472 - DNA, 120 bp, Artificial Sequence
  degradation probe
atctttatttttattaaaggacagggtctctggcctggtgcgttggctcacgcctgtaatcccagcactttgggaagccaaggcgggcggatcacgaggtcaggagattgagaccatcct

SEQ ID NO: 473 - DNA, 120 bp, Artificial Sequence
  degradation probe
tgggaagccaaggcgggcggatcacgaggtcaggagattgagaccatcctggccaacacgatgaaaccccgtctctactaaaaatacaaaaattagctgggcgtggtggtgagtgcctgt

SEQ ID NO: 474 - DNA, 120 bp, Artificial Sequence
  degradation probe
gtctctactaaaaatacaaaaattagctgggcgtggtggtgagtgcctgtagtccctgctactcaggaggctgaggcaggagacttgctcctgaacccaagaggcggaggttgcagtgag

SEQ ID NO: 475 - DNA, 120 bp, Artificial Sequence
  degradation probe
ctgaggcaggagacttgctcctgaacccaagaggcggaggttgcagtgagccaagatcccgccactgcactccagcttcccgacagagcgagactctgtctcacaaaaaaaaaaaaaaaa

SEQ ID NO: 476 - DNA, 120 bp, Artificial Sequence
  degradation probe
tccagcttcccgacagagcgagactctgtctcacaaaaaaaaaaaaaaaaaaaaaaaaaaagtcagggtctcaccctgtccgccaggcttgactgcagtggcgtgacctcagcttactgc

SEQ ID NO: 477 - DNA, 120 bp, Artificial Sequence
  degradation probe
tcaccctgtccgccaggcttgactgcagtggcgtgacctcagcttactgcagcctcaacctcccagactcaagtgatcctcccacctcagcctcctgagtagctgggactacaggtgcac

SEQ ID NO: 478 - DNA, 120 bp, Artificial Sequence
  degradation probe
gataccagcctggacaacatagcaagaccccatctctaaaaaaatataaaaattagccaggcatagcagtgtgcacctgtagtcccagctactcaggaggctgaggtgggaggatcactt

SEQ ID NO: 479 - DNA, 120 bp, Artificial Sequence
  degradation probe
cgtgactcacatctgtgatcccagcactttggcaggctgaggcaggaggatcactggaggccaggagcttgataccagcctggacaacatagcaagaccccatctctaaaaaaatataaa

SEQ ID NO: 480 - DNA, 120 bp, Artificial Sequence
  degradation probe
caggctggtatcaagctcctggcctccagtgatcctcctgcctcagcctgccaaagtgctgggatcacagatgtgagtcacgtgcttggcctaaacttttaaatgatgagacttaagtca

SEQ ID NO: 481 - DNA, 120 bp, Artificial Sequence
  degradation probe
ctcagtttccccttttgtaaaataggatgatgatacttgcacctcaaggtgctgggaggattcactgtgagcatgtgagaagcagagggcagactgtggtggctggtgggccagggcaga

SEQ ID NO: 482 - DNA, 120 bp, Artificial Sequence
  degradation probe
cacacctgcgctccagcctgggtgacagagcgagactccgtctcaaaaaaaaataaaaataaaaaataaactcagtttccccttttgtaaaataggatgatgatacttgcacctcaaggt

SEQ ID NO: 483 - DNA, 120 bp, Artificial Sequence
  degradation probe
ttttttgagacggagtctcgctctgtcacccaggctggagcgcaggtgtgcgatctcggctcactgcaacctccacctcctgggttcaagcgattctcctgcctcagcctcctgagtagc

SEQ ID NO: 484 - DNA, 120 bp, Artificial Sequence
  degradation probe
cggtgaaaccccatctctactaaaaatacaaaaaattagctgggtgtggtggtgtgtgcctgtagtcccagctactcaggaggctgaggcaggagaatcgcttgaacccaggaggtggag

SEQ ID NO: 485 - DNA, 120 bp, Artificial Sequence
  degradation probe
atcccagcactttgggaggccgaggcggtcggatcacgaggtcaggagatcaagaccatcctggctaacacggtgaaaccccatctctactaaaaatacaaaaaattagctgggtgtggt

SEQ ID NO: 486 - DNA, 120 bp, Artificial Sequence
  degradation probe
cctgggcaaatcacttcctttctttaaattcagtttccccttttgccgggcgcggtggctcatgcttgtaatcccagcactttgggaggccgaggcggtcggatcacgaggtcaggagat

SEQ ID NO: 487 - DNA, 120 bp, Artificial Sequence
  degradation probe
gggtctgcaggctgggtggaccccaagcttagttggatcctgggcaaatcacttcctttctttaaattcagtttccccttttgccgggcgcggtggctcatgcttgtaatcccagcactt

SEQ ID NO: 488 - DNA, 120 bp, Artificial Sequence
  degradation probe
cccggcctggggggtcaggagggcttcctggaagagggggtatcctgagccctggaagaggagacaccagccaggctgctagaggctggggatccccagcacacaggctccaggctgggc

SEQ ID NO: 489 - DNA, 120 bp, Artificial Sequence
  degradation probe
tagcagcctggctggtgtctcctcttccagggctcaggataccccctcttccaggaagccctcctgaccccccaggccgggcagccactccagggctcctcagttcagcaatgtgtctct

SEQ ID NO: 490 - DNA, 120 bp, Artificial Sequence
  degradation probe
ttcaagtgattcttcttgcctcagcctcccaagtagctgggattacaggcacccacgaccatgcccggctaatttttgtatttttagtagagacagggtttcaccatattggccaggctg

SEQ ID NO: 491 - DNA, 120 bp, Artificial Sequence
  degradation probe
aatttttgtatttttagtagagacagggtttcaccatattggccaggctggtctcgaactcctgacctcgtgatcctcctgccttggcctcccaaagtgttgggattacaggcatgagcc

SEQ ID NO: 492 - DNA, 120 bp, Artificial Sequence
  degradation probe
tgatcctcctgccttggcctcccaaagtgttgggattacaggcatgagccaccgtgcccggcctaatttttgtattttttagtagaaacagggttccaccatgttggtcaggctggtctt

SEQ ID NO: 493 - DNA, 120 bp, Artificial Sequence
  degradation probe
tgtattttttagtagaaacagggttccaccatgttggtcaggctggtcttgaggtcctgaccacaggtgatctgacctcgccttggcctcgaaaagtgctgggattataggcatgagcca

SEQ ID NO: 494 - DNA, 120 bp, Artificial Sequence
  degradation probe
tctgacctcgccttggcctcgaaaagtgctgggattataggcatgagccaccgcaccccacaccaacctagaaaagttcaactcatttgtctaagcccagcaaaatcatccagaagccgg

SEQ ID NO: 495 - DNA, 120 bp, Artificial Sequence
  degradation probe
gaaaagttcaactcatttgtctaagcccagcaaaatcatccagaagccggtggcggtggctcacacccataatgccagcactttgggaggctaagacatgaggatcacttgaggccaaga

SEQ ID NO: 496 - DNA, 120 bp, Artificial Sequence
  degradation probe
aatgccagcactttgggaggctaagacatgaggatcacttgaggccaagagtttgagaccagcctgagaaatatagcaagacccccgtctctatgaaaaatacaaaaattagttgggtgc

SEQ ID NO: 497 - DNA, 120 bp, Artificial Sequence
  degradation probe
atatagcaagacccccgtctctatgaaaaatacaaaaattagttgggtgcggcggcatgtgcctgtggtcccaactactctggagactgaggcaggaggatcacttgaggccaggagctt

SEQ ID NO: 498 - DNA, 120 bp, Artificial Sequence
  degradation probe
ccaactactctggagactgaggcaggaggatcacttgaggccaggagcttgaggctgcagtgatctgtgatggtgccactgcactccagccagggcgacagaatgacactgtctcaaaaa

SEQ ID NO: 499 - DNA, 120 bp, Artificial Sequence
  degradation probe
tggtgccactgcactccagccagggcgacagaatgacactgtctcaaaaaaaaatgtcggctgggcgcggtggctcacacctgtaatcccagcacttaaggaggctgaggtgggcggatc

SEQ ID NO: 500 - DNA, 120 bp, Artificial Sequence
  degradation probe
tggctcacacctgtaatcccagcacttaaggaggctgaggtgggcggatcacgaggtcaggagatcgagaccatcctggctaacacggtgaaaccccgtctctactaaaaatacaaaaaa

SEQ ID NO: 501 - DNA, 120 bp, Artificial Sequence
  degradation probe
ccatcctggctaacacggtgaaaccccgtctctactaaaaatacaaaaaattagcctggcgtggtggcgggcgcctgtagtcccagctactcgggaggctgaggcaggagaatggcatga

SEQ ID NO: 502 - DNA, 120 bp, Artificial Sequence
  degradation probe
gcgcctgtagtcccagctactcgggaggctgaggcaggagaatggcatgaacccgggaggcggagctggcagtgagccgagatcgtgtcactacactctagcctggatgacagggccaga

SEQ ID NO: 503 - DNA, 120 bp, Artificial Sequence
  degradation probe
aggcggagctggcagtgagccgagatcgtgtcactacactctagcctggatgacagggccagactccatctcaaaaaaaaaaaaaaaaaaatcatctagtggtgatgggactttgtaggg

SEQ ID NO: 504 - DNA, 120 bp, Artificial Sequence
  degradation probe
ggagaattacagcccccatcatagtgtgttggggtaagaattaagtgagttcccatatatgggacaaacttcccccccttatgacctgtaaacccagtttctggcggccaaggatgggga

SEQ ID NO: 505 - DNA, 120 bp, Artificial Sequence
  degradation probe
gcactccagcctgggtgacagagtgagactccatctcaaaaataaaatcaataaaataaagtaaaatgagggagaattacagcccccatcatagtgtgttggggtaagaattaagtgagt

SEQ ID NO: 506 - DNA, 120 bp, Artificial Sequence
  degradation probe
tgattttatttttgagatggagtctcactctgtcacccaggctggagtgcaatggcatgaacttggctcactgcaacctctgactcccaggttcaagagattctcttgcctcagcctcct

SEQ ID NO: 507 - DNA, 120 bp, Artificial Sequence
  degradation probe
ctgcaacctctgactcccaggttcaagagattctcttgcctcagcctcctcagtagctggaaccacaggcgcccgccaccacacctggctaatttttgtatttttagtacagactgggtt

SEQ ID NO: 508 - DNA, 120 bp, Artificial Sequence
  degradation probe
gcccgccaccacacctggctaatttttgtatttttagtacagactgggtttcaccgtgttggccaggctggtctcaaactcctgacttcaagtgatccgcctgccttggcctcccaaagt

SEQ ID NO: 509 - DNA, 120 bp, Artificial Sequence
  degradation probe
gtctcaaactcctgacttcaagtgatccgcctgccttggcctcccaaagtgctaggattacaggtgtgagccaccatgtccagtcctctccccattttataggtgaggagactgaggctc

SEQ ID NO: 510 - DNA, 120 bp, Artificial Sequence
  degradation probe
ccaccatgtccagtcctctccccattttataggtgaggagactgaggctcagagaagtaaagctactcacctaagatcccacagccttcaagcggcagagctgggatttgaacctcggcc

SEQ ID NO: 511 - DNA, 120 bp, Artificial Sequence
  degradation probe
tactcacctaagatcccacagccttcaagcggcagagctgggatttgaacctcggccatctgggtgtggagccttattattcaccactttggcaggtgtctcagtggcttacttaccctt

SEQ ID NO: 512 - DNA, 120 bp, Artificial Sequence
  degradation probe
cccagctcatgcagtcatttattcattcaacaagcatcacccacgctgggcccccaagaggcctcagccctgaccaggtcctcaaggagctatcttaagagaggacagagctggacatag

SEQ ID NO: 513 - DNA, 120 bp, Artificial Sequence
  degradation probe
tgaccaggtcctcaaggagctatcttaagagaggacagagctggacatagacactcctagtcccatgcagttaaggaaggtccagagggaaagacagggtcctggaaggcttcctggagg

SEQ ID NO: 514 - DNA, 120 bp, Artificial Sequence
  degradation probe
ccaatgataggtttggggaatcttagtgaactcctactcatgcgttgaagccccaactgtaatgcctcctcctccaggaagccttccaggaccctgtctttccctctggaccttccttaa

SEQ ID NO: 515 - DNA, 120 bp, Artificial Sequence
  degradation probe
ttggagagccaatgataggtttggggaatcttagtgaactcctactcatgcgttgaagccccaactgtaatgcctcctcctccaggaagccttccaggaccctgtctttccctctggacc

SEQ ID NO: 516 - DNA, 120 bp, Artificial Sequence
  degradation probe
cctctgtccttcacatcgccaaactccaagaaatggtcaaaggctttgaacggagaccttgaagaaaaagcgggaaactgcttttcaacatgtccagagatgctcattcttgccaggtgc

SEQ ID NO: 517 - DNA, 120 bp, Artificial Sequence
  degradation probe
cgggaaactgcttttcaacatgtccagagatgctcattcttgccaggtgcagtggcttacacctgtaatcccaacactttgggaggctgaagtgagcagatcgcttgagcccaggagttc

SEQ ID NO: 518 - DNA, 120 bp, Artificial Sequence
  degradation probe
ccaacactttgggaggctgaagtgagcagatcgcttgagcccaggagttcgagaccagcctggccatcatggtgaaaccccgtctctactttaaaaaatacaaaaattagccaggtgtgg

SEQ ID NO: 519 - DNA, 120 bp, Artificial Sequence
  degradation probe
ggtgaaaccccgtctctactttaaaaaatacaaaaattagccaggtgtggtggtatgtgcctgtagtcccagctactcaggaggctgaggcaggagaattgtttgaacccaggaggtgga

SEQ ID NO: 520 - DNA, 120 bp, Artificial Sequence
  degradation probe
agctactcaggaggctgaggcaggagaattgtttgaacccaggaggtggaggctgcagtgagccaagatcactccactgcattccagcctgggcaacagagtgagacctgtctcaaaaaa

SEQ ID NO: 521 - DNA, 120 bp, Artificial Sequence
  degradation probe
actccactgcattccagcctgggcaacagagtgagacctgtctcaaaaaaaaaaaaaagttcacacttaataagaaaactgcaaatcacaaactgtttgtcttcaccaatcaaattggca

SEQ ID NO: 522 - DNA, 120 bp, Artificial Sequence
  degradation probe
caccagtgctacaccatggacgcaccccacagcctggctggcacagaatgtcattgcattgttggatttttgccaatttgattggtgaagacaaacagtttgtgatttgcagttttctta

SEQ ID NO: 523 - DNA, 120 bp, Artificial Sequence
  degradation probe
cagaagtggacttgctgtgtcagcaggcacatgagtttatcattccggtggcctgtgccagggtgccctccaccagtgctacaccatggacgcaccccacagcctggctggcacagaatg

SEQ ID NO: 524 - DNA, 120 bp, Artificial Sequence
  degradation probe
caccggaatgataaactcatgtgcctgctgacacagcaagtccacttctgggaatccctgcctgaatatttgcatgtttacagtgtgaactgactaacgaataaaaaaatgtggctcagc

SEQ ID NO: 525 - DNA, 120 bp, Artificial Sequence
  degradation probe
ctgacctcgtgatccgcccaccttggtcccccaaagtgctgggattacaggcatgagccaccacgtccaggctgagccacatttttttattcgttagtcagttcacactgtaaacatgca

SEQ ID NO: 526 - DNA, 120 bp, Artificial Sequence
  degradation probe
tgcccagctaatttttgtatttttagtggagacggggtttcaccatgttggccaggctggtctggtactcctgacctcgtgatccgcccaccttggtcccccaaagtgctgggattacag

SEQ ID NO: 527 - DNA, 120 bp, Artificial Sequence
  degradation probe
caacatggtgaaaccccgtctccactaaaaatacaaaaattagctgggcatggtggcacgtgcctgtaatcccagctactagggaggctgaggcaggagaatcgcttgaacccaggaggc

SEQ ID NO: 528 - DNA, 120 bp, Artificial Sequence
  degradation probe
cccagctactagggaggctgaggcaggagaatcgcttgaacccaggaggcagaggttgcagtgagctgagatcatgccactgcactccagtttgggcgttagagcgagactccatctcaa

SEQ ID NO: 529 - DNA, 120 bp, Artificial Sequence
  degradation probe
atcatgccactgcactccagtttgggcgttagagcgagactccatctcaataataataataataataataataataataatttaaaaattagctgggcatggtggcttcagcctgtagtc

SEQ ID NO: 530 - DNA, 120 bp, Artificial Sequence
  degradation probe
ataataataatttaaaaattagctgggcatggtggcttcagcctgtagtcccagctacttcagaggctgaggtgggaggatcccttgagcccagaaggttgtgactgcagtgagccgtga

SEQ ID NO: 531 - DNA, 120 bp, Artificial Sequence
  degradation probe
ggtgggaggatcccttgagcccagaaggttgtgactgcagtgagccgtgattgtgccactccactccagcctatgcagcagagcaagaccctatctctaaaaaataatttaaaataaatg

SEQ ID NO: 532 - DNA, 120 bp, Artificial Sequence
  degradation probe
ctatgcagcagagcaagaccctatctctaaaaaataatttaaaataaatggctgggccggccgcggtggctcatgcctgtaatcccagcactttgggaggctgaggtgggcggatcacct

SEQ ID NO: 533 - DNA, 120 bp, Artificial Sequence
  degradation probe
tcatgcctgtaatcccagcactttgggaggctgaggtgggcggatcacctgaggtccagagtttgagactagcctgaccaacgtggagaaactccatctctactaaatacacaaaattag

SEQ ID NO: 534 - DNA, 120 bp, Artificial Sequence
  degradation probe
agcctgaccaacgtggagaaactccatctctactaaatacacaaaattagccgggtgtggtggcgcatgcctgtaatcccagctacttgggaggctgaggcaggagaatcacttgagcct

SEQ ID NO: 535 - DNA, 120 bp, Artificial Sequence
  degradation probe
ctgtaatcccagctacttgggaggctgaggcaggagaatcacttgagcctgggaggtagagtttgcagttgagccgagatcacggcactgcactccagcctgggcaacaagagcaaaact

SEQ ID NO: 536 - DNA, 120 bp, Artificial Sequence
  degradation probe
gagccgagatcacggcactgcactccagcctgggcaacaagagcaaaactccatctcaaaataataataataaataaataaattggctaaaatgttcaattttatgttgtgtgtatgttg

SEQ ID NO: 537 - DNA, 120 bp, Artificial Sequence
  degradation probe
taaataaataaattggctaaaatgttcaattttatgttgtgtgtatgttgcaggtttgtttttttgagaaggcaaagtcaggcgtggtggctcacacctgtaatcccagcactttgggag

SEQ ID NO: 538 - DNA, 120 bp, Artificial Sequence
  degradation probe
ggcaaagtcaggcgtggtggctcacacctgtaatcccagcactttgggaggctgaggtgggcggatcacaaggtcaggagactgagacaatcctggccaacgctgtgaaaccctgtctct

SEQ ID NO: 539 - DNA, 120 bp, Artificial Sequence
  degradation probe
aggtcaggagactgagacaatcctggccaacgctgtgaaaccctgtctctaccaaaaatacaaaaaattagctgggcgtggtggcatgtacctgtaatcccagctactcgggaggctgag

SEQ ID NO: 540 - DNA, 120 bp, Artificial Sequence
  degradation probe
gctgggcgtggtggcatgtacctgtaatcccagctactcgggaggctgaggcaggaaaatcacttgaaccagggagttggaggttgcagtaagcctagatcgcgccacagcactccagcc

SEQ ID NO: 541 - DNA, 120 bp, Artificial Sequence
  degradation probe
tcacttgaaccagggagttggaggttgcagtaagcctagatcgcgccacagcactccagcctggtgacagagcgagacttcatataaaaaaaaaaaagagagagagagagaaggcaaaaa

SEQ ID NO: 542 - DNA, 120 bp, Artificial Sequence
  degradation probe
tactaaaaatacaaaaaaattagctgggcatggtggcgggcgcctgtagtcccagctacttgggaggctgaggcagaagaatggcttgaacccgggaggtggagcttgcagtgagctgag

SEQ ID NO: 543 - DNA, 120 bp, Artificial Sequence
  degradation probe
aggcagaagaatggcttgaacccgggaggtggagcttgcagtgagctgagattgcgccactgcactccagcctgagcgacagagcgagactccatctcaaaacaaaacaaacaaacaaaa

SEQ ID NO: 544 - DNA, 120 bp, Artificial Sequence
  degradation probe
ctgcactccagcctgagcgacagagcgagactccatctcaaaacaaaacaaacaaacaaaacaaaacaaaacaaaacaaaacaaaaaaagattcctttggctacctgggtgggtagccca

SEQ ID NO: 545 - DNA, 120 bp, Artificial Sequence
  degradation probe
cttcacaagatgcaatgaagaagtgggctttttttttttttgagacagagtttctctcttgtcacccaggctggagtgccatggcgcaatctcagctcactgcaacctccacttcccggg

SEQ ID NO: 546 - DNA, 120 bp, Artificial Sequence
  degradation probe
ctggagtgccatggcgcaatctcagctcactgcaacctccacttcccgggttcaagcgactctcctgcctcagctacctgggtgactgggattacaggcttgcgccaccatgcccggcta

SEQ ID NO: 547 - DNA, 120 bp, Artificial Sequence
  degradation probe
ctgaggtcaggagtttgagaccagcctgaccaacatggagaaaccccgtctctactaaaaatacataaattagccgggcatggtggcgcaagcctgtaatcccagtcacccaggtagctg

SEQ ID NO: 548 - DNA, 120 bp, Artificial Sequence
  degradation probe
acttctggccgggtgtggtggctcatgcctgtaatcccagcactttgggaggctgaggcgggtggatcacctgaggtcaggagtttgagaccagcctgaccaacatggagaaaccccgtc

SEQ ID NO: 549 - DNA, 120 bp, Artificial Sequence
  degradation probe
tcccaaagtgctgggattacaggcatgagccaccacacccggccagaagtaggcctttacccactacatgccttcttttacttatctgcacaatgaagattaaaaatagtttcttggcct

SEQ ID NO: 550 - DNA, 120 bp, Artificial Sequence
  degradation probe
ccttcttttacttatctgcacaatgaagattaaaaatagtttcttggcctggcatggtgcctcacacctgtaattccagcactttgggaggctgaggtgggcagatcatttgaggtcaga

SEQ ID NO: 551 - DNA, 120 bp, Artificial Sequence
  degradation probe
taattccagcactttgggaggctgaggtgggcagatcatttgaggtcagaagttcaagaccagtgtggccaacgtggcgaaaccccatctctactaaaaatacaaaaagtagccaggcat

SEQ ID NO: 552 - DNA, 120 bp, Artificial Sequence
  degradation probe
aacgtggcgaaaccccatctctactaaaaatacaaaaagtagccaggcatggtggcacgtgcctgtagtcccagacactcaggagactgaggcacaagaatctcttgaacctgggagatg

SEQ ID NO: 553 - DNA, 120 bp, Artificial Sequence
  degradation probe
tttgagacagagtcttgctctgtcccccaggctggagtgcaatggctcgatgtcagctcactgcaacctccatctcccaggttcaagagattcttgtgcctcagtctcctgagtgtctgg

SEQ ID NO: 554 - DNA, 120 bp, Artificial Sequence
  degradation probe
gtatcaattaaacatggagcaatttaattctcacagtaaccctatgataagagacttttttttttttttttttgagacagagtcttgctctgtcccccaggctggagtgcaatggctcga

SEQ ID NO: 555 - DNA, 120 bp, Artificial Sequence
  degradation probe
ttatcatagggttactgtgagaattaaattgctccatgtttaattgatacatgataaactgatacttagcacagggcctagtaaatatcagtgctctagtagtgcctgcccatattatta

SEQ ID NO: 556 - DNA, 120 bp, Artificial Sequence
  degradation probe
acagggcctagtaaatatcagtgctctagtagtgcctgcccatattattatcattgtttgtgtcatttatctctgtagtaggaagtatatacaatcctattgcataggtttaagggaatt

SEQ ID NO: 557 - DNA, 120 bp, Artificial Sequence
  degradation probe
ctctgtagtaggaagtatatacaatcctattgcataggtttaagggaattaatccactaaagtgtttggaacagagcctggcacatattgctatgtaactgcatcatcatcatcatccct

SEQ ID NO: 558 - DNA, 120 bp, Artificial Sequence
  degradation probe
acagagcctggcacatattgctatgtaactgcatcatcatcatcatccctttttaatagatgagaaaactgaatttcagagagggtagtctgatctgaaattcacctattattccagctt

SEQ ID NO: 559 - DNA, 120 bp, Artificial Sequence
  degradation probe
aactgcatcatcatcatcatccctttttaatagatgagaaaactgaatttcagagagggtagtctgatctgaaattcacctattattccagcttctctcttccaaatacggcctctgtgc

SEQ ID NO: 560 - DNA, 120 bp, Artificial Sequence
  degradation probe
agaattctcattcagaaccttgatcacaggccgggcgcgccgggattgctcacgcctgtaatcccaacactttgggaggccgaggtgggaggatcacttgagcccaggatttggagacta

SEQ ID NO: 561 - DNA, 120 bp, Artificial Sequence
  degradation probe
tttgggaggccgaggtgggaggatcacttgagcccaggatttggagactagcctgggcaacatagcgagaccccttctctaaaaaagcaaacaggcaaaacttcatgagaatcttgatca

SEQ ID NO: 562 - DNA, 120 bp, Artificial Sequence
  degradation probe
ccccttctctaaaaaagcaaacaggcaaaacttcatgagaatcttgatcatgttaaaattttatgtccttcgatttctccctacacacacacacacacacacacacacacacacacacac

SEQ ID NO: 563 - DNA, 120 bp, Artificial Sequence
  degradation probe
tgagaatcttgatcatgttaaaattttatgtccttcgatttctccctacacacacacacacacacacacacacacacacacacacacacactcaacatttcctccacccatatcatcact

SEQ ID NO: 564 - DNA, 120 bp, Artificial Sequence
  degradation probe
tagttttatcctgttttccgaaaaacaatcatttatttatttatttatttatttaattttatgagacagggtctggctttgtcacccaggctggagtgcagtggtgcgatcttggctcac

SEQ ID NO: 565 - DNA, 120 bp, Artificial Sequence
  degradation probe
gtctggctttgtcacccaggctggagtgcagtggtgcgatcttggctcactgcaacctctgcctctcagattcaagccatccttccacctcagctctgccactgagtagctgagactaca

SEQ ID NO: 566 - DNA, 120 bp, Artificial Sequence
  degradation probe
ttcaagccatccttccacctcagctctgccactgagtagctgagactacaagcactcgccaccatgcccggctaattaaaaaaataataatcattttaaatgcaagctttatattataaa

SEQ ID NO: 567 - DNA, 120 bp, Artificial Sequence
  degradation probe
gctaattaaaaaaataataatcattttaaatgcaagctttatattataaatacaaagtaaacatgaaaataaaacccaaacatagcagtgttattaaactctggcctgtagcagtggctc

SEQ ID NO: 568 - DNA, 120 bp, Artificial Sequence
  degradation probe
aaaacccaaacatagcagtgttattaaactctggcctgtagcagtggctcacacctgtaatcctagcagtttggaggccgagacaggtggattacttgagacctggagtttgagaccagc

SEQ ID NO: 569 - DNA, 120 bp, Artificial Sequence
  degradation probe
ttggaggccgagacaggtggattacttgagacctggagtttgagaccagcccaggtgacacagcaagacctcatctctactaaaaataaaaaaaaattagccaggtgtggtggtatgcac

SEQ ID NO: 570 - DNA, 120 bp, Artificial Sequence
  degradation probe
tcatctctactaaaaataaaaaaaaattagccaggtgtggtggtatgcacctgtggtcccagctacttaggatgctggagtgcgaggatcgcttgagcccaggaggtcaaggctgcagtg

SEQ ID NO: 571 - DNA, 120 bp, Artificial Sequence
  degradation probe
gatgctggagtgcgaggatcgcttgagcccaggaggtcaaggctgcagtgaactatgatcactcattacaccccagcctgggtgacagagcgagatgctgtctcaaaacaaaacaaaacg

SEQ ID NO: 572 - DNA, 120 bp, Artificial Sequence
  degradation probe
ccccagcctgggtgacagagcgagatgctgtctcaaaacaaaacaaaacgaaaaacaactctggctagatgctattgcttgccaagggtgcagtcttccatttattaaaagtgaaaatta

SEQ ID NO: 573 - DNA, 120 bp, Artificial Sequence
  degradation probe
gctattgcttgccaagggtgcagtcttccatttattaaaagtgaaaattagggccaggcacattggctcatgcctgtaatcccagcactttgggaggctgaggtgggtggatcacctgag

SEQ ID NO: 574 - DNA, 120 bp, Artificial Sequence
  degradation probe
tgcctgtaatcccagcactttgggaggctgaggtgggtggatcacctgaggtcaggagttcgagaccagcctggccaacatggtgaaaccttatctctgccaaaaatataaaagattagc

SEQ ID NO: 575 - DNA, 120 bp, Artificial Sequence
  degradation probe
ctggccaacatggtgaaaccttatctctgccaaaaatataaaagattagccatgtgtcgtggtgggtgcttgtaatctcagctacttgggaggctgaggcaggagaatcacttgaaccca

SEQ ID NO: 576 - DNA, 120 bp, Artificial Sequence
  degradation probe
tgtaatctcagctacttgggaggctgaggcaggagaatcacttgaacccaggaggcagaggttgcagtgagccaagattgtgccattgcactccagcctgtgcaacgagcgaaactccaa

SEQ ID NO: 577 - DNA, 120 bp, Artificial Sequence
  degradation probe
tcagctacttgggaggctgaggcaggagaatcacttgaacccaggaggcagaggttgcagtgagccaagattgtgccattgcactccagcctgtgcaacgagcgaaactccaactcaaaa